SEQUENCE LISTING (38 sequences)

SEQ ID NO: 1 - DNA, 2577 bp, Artificial Sequence
  Codon optimized DNA encoding modified wt pol
agatctaccatggcccccatctcccccattgagactgtgcctgtgaagctgaagcctggcatggatggccccaaggtgaagcagtggcccctgactgaggagaagatcaaggccctggtggaaatctgcactgagatggagaaggagggcaaaatctccaagattggccccgagaacccctacaacacccctgtgtttgccatcaagaagaaggactccaccaagtggaggaagctggtggacttcagggagctgaacaagaggacccaggacttctgggaggtgcagctgggcatcccccaccccgctggcctgaagaagaagaagtctgtgactgtgctggatgtgggggatgcctacttctctgtgcccctggatgaggacttcaggaagtacactgccttcaccatcccctccatcaacaatgagacccctggcatcaggtaccagtacaatgtgctgccccagggctggaagggctcccctgccatcttccagtcctccatgaccaagatcctggagcccttcaggaagcagaaccctgacattgtgatctaccagtacatggatgacctgtatgtgggctctgacctggagattgggcagcacaggaccaagattgaggagctgaggcagcacctgctgaggtggggcctgaccacccctgacaagaagcaccagaaggagccccccttcctgtggatgggctatgagctgcaccccgacaagtggactgtgcagcccattgtgctgcctgagaaggactcctggactgtgaatgacatccagaagctggtgggcaagctgaactgggcctcccaaatctaccctggcatcaaggtgaggcagctgtgcaagctgctgaggggcaccaaggccctgactgaggtgatccccctgactgaggaggctgagctggagctggctgagaacagggagatcctgaaggagcctgtgcatggggtgtactatgacccctccaaggacctgattgctgagatccagaagcagggccagggccagtggacctaccaaatctaccaggagcccttcaagaacctgaagactggcaagtatgccaggatgaggggggcccacaccaatgatgtgaagcagctgactgaggctgtgcagaagatcaccactgagtccattgtgatctggggcaagacccccaagttcaagctgcccatccagaaggagacctgggagacctggtggactgagtactggcaggccacctggatccctgagtgggagtttgtgaacaccccccccctggtgaagctgtggtaccagctggagaaggagcccattgtgggggctgagaccttctatgtggatggggctgccaacagggagaccaagctgggcaaggctggctatgtgaccaacaggggcaggcagaaggtggtgaccctgactgacaccaccaaccagaagactgagctccaggccatctacctggccctccaggactctggcctggaggtgaacattgtgactgactcccagtatgccctgggcatcatccaggcccagcctgatcagtctgagtctgagctggtgaaccagatcattgagcagctgatcaagaaggagaaggtgtacctggcctgggtgcctgcccacaagggcattgggggcaatgagcaggtggacaagctggtgtctgctggcatcaggaaggtgctgttcctggatggcattgacaaggcccaggatgagcatgagaagtaccactccaactggagggctatggcctctgacttcaacctgccccctgtggtggctaaggagattgtggcctcctgtgacaagtgccagctgaagggggaggccatgcatgggcaggtggactgctcccctggcatctggcagctggactgcacccacctggagggcaaggtgatcctggtggctgtgcatgtggcctccggctacattgaggctgaggtgatccctgctgagacaggccaggagactgcctacttcctgctgaagctggctggcaggtggcctgtgaagaccatccacactgacaatggctccaacttcactggggccacagtgagggctgcctgctggtgggctggcatcaagcaggagtttggcatcccctacaacccccagtcccagggggtggtggagtccatgaacaaggagctgaagaagatcattgggcaggtgagggaccaggctgagcacctgaagacagctgtgcagatggctgtgttcatccacaacttcaagaggaaggggggcatcgggggctactccgctggggagaggattgtggacatcattgccacagacatccagaccaaggagctccagaagcagatcaccaagatccagaacttcagggtgtactacagggactccaggaaccccctgtggaagggccctgccaagctgctgtggaagggggagggggctgtggtgatccaggacaactctgacatcaaggtggtgcccaggaggaaggccaagatcatcagggactatggcaagcagatggctggggatgactgtgtggcctccaggcaggatgaggactaaagcccgggcagatct

SEQ ID NO: 2 - protein, 850 aa, Artificial Sequence
  Codon optimized HIV-1 pol
MAPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTIHTDNGSNFTGATVRAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 3 - DNA, 2577 bp, Artificial Sequence
  Codon optimized DNA encoding inactivated Pol (IA-Pol)
agatctaccatggcccccatctcccccattgagactgtgcctgtgaagctgaagcctggcatggatggccccaaggtgaagcagtggcccctgactgaggagaagatcaaggccctggtggaaatctgcactgagatggagaaggagggcaaaatctccaagattggccccgagaacccctacaacacccctgtgtttgccatcaagaagaaggactccaccaagtggaggaagctggtggacttcagggagctgaacaagaggacccaggacttctgggaggtgcagctgggcatcccccaccccgctggcctgaagaagaagaagtctgtgactgtgctggctgtgggggatgcctacttctctgtgcccctggatgaggacttcaggaagtacactgccttcaccatcccctccatcaacaatgagacccctggcatcaggtaccagtacaatgtgctgccccagggctggaagggctcccctgccatcttccagtcctccatgaccaagatcctggagcccttcaggaagcagaaccctgacattgtgatctaccagtacatggctgccctgtatgtgggctctgacctggagattgggcagcacaggaccaagattgaggagctgaggcagcacctgctgaggtggggcctgaccacccctgacaagaagcaccagaaggagccccccttcctgtggatgggctatgagctgcaccccgacaagtggactgtgcagcccattgtgctgcctgagaaggactcctggactgtgaatgacatccagaagctggtgggcaagctgaactgggcctcccaaatctaccctggcatcaaggtgaggcagctgtgcaagctgctgaggggcaccaaggccctgactgaggtgatccccctgactgaggaggctgagctggagctggctgagaacagggagatcctgaaggagcctgtgcatggggtgtactatgacccctccaaggacctgattgctgagatccagaagcagggccagggccagtggacctaccaaatctaccaggagcccttcaagaacctgaagactggcaagtatgccaggatgaggggggcccacaccaatgatgtgaagcagctgactgaggctgtgcagaagatcaccactgagtccattgtgatctggggcaagacccccaagttcaagctgcccatccagaaggagacctgggagacctggtggactgagtactggcaggccacctggatccctgagtgggagtttgtgaacaccccccccctggtgaagctgtggtaccagctggagaaggagcccattgtgggggctgagaccttctatgtggctggggctgccaacagggagaccaagctgggcaaggctggctatgtgaccaacaggggcaggcagaaggtggtgaccctgactgacaccaccaaccagaagactgccctccaggccatctacctggccctccaggactctggcctggaggtgaacattgtgactgcctcccagtatgccctgggcatcatccaggcccagcctgatcagtctgagtctgagctggtgaaccagatcattgagcagctgatcaagaaggagaaggtgtacctggcctgggtgcctgcccacaagggcattgggggcaatgagcaggtggacaagctggtgtctgctggcatcaggaaggtgctgttcctggatggcattgacaaggcccaggatgagcatgagaagtaccactccaactggagggctatggcctctgacttcaacctgccccctgtggtggctaaggagattgtggcctcctgtgacaagtgccagctgaagggggaggccatgcatgggcaggtggactgctcccctggcatctggcagctggcctgcacccacctggagggcaaggtgatcctggtggctgtgcatgtggcctccggctacattgaggctgaggtgatccctgctgagacaggccaggagactgcctacttcctgctgaagctggctggcaggtggcctgtgaagaccatccacactgccaatggctccaacttcactggggccacagtgagggctgcctgctggtgggctggcatcaagcaggagtttggcatcccctacaacccccagtcccagggggtggtggcctccatgaacaaggagctgaagaagatcattgggcaggtgagggaccaggctgagcacctgaagacagctgtgcagatggctgtgttcatccacaacttcaagaggaaggggggcatcgggggctactccgctggggagaggattgtggacatcattgccacagacatccagaccaaggagctccagaagcagatcaccaagatccagaacttcagggtgtactacagggactccaggaaccccctgtggaagggccctgccaagctgctgtggaagggggagggggctgtggtgatccaggacaactctgacatcaaggtggtgcccaggaggaaggccaagatcatcagggactatggcaagcagatggctggggatgactgtgtggcctccaggcaggatgaggactaaagcccgggcagatct

SEQ ID NO: 4 - protein, 850 aa, Artificial Sequence
  Codon optimized DNA inactivated Pol (IA-Pol)
MAPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLAVGDAYFSVPLDEDFRKYTAFTIPSINNETPGIRYQYNVLPQGWKGSPAIFQSSMTKILEPFRKQNPDIVIYQYMAALYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVAGAANRETKLGKAGYVTNRGRQKVVTLTDTTNQKTALQAIYLALQDSGLEVNIVTASQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLACTHLEGKVILVAVHVASGYIEAEVIPAETGQETAYFLLKLAGRWPVKTIHTANGSNFTGATVRAACWWAGIKQEFGIPYNPQSQGVVASMNKELKKIIGQVRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 5 - DNA, 2650 bp, Artificial Sequence
  Codon optimized DNA encoding modified HIV-1 pol fused to an amino terminal localized leader sequence
gatcaccatggatgcaatgaagagagggctctgctgtgtgctgctgctgtgtggagcagtcttcgtttcgcccagcgagatctccgcccccatctcccccattgagactgtgcctgtgaagctgaagcctggcatggatggccccaaggtgaagcagtggcccctgactgaggagaagatcaaggccctggtggaaatctgcactgagatggagaaggagggcaaaatctccaagattggccccgagaacccctacaacacccctgtgtttgccatcaagaagaaggactccaccaagtggaggaagctggtggacttcagggagctgaacaagaggacccaggacttctgggaggtgcagctgggcatcccccaccccgctggcctgaagaagaagaagtctgtgactgtgctggatgtgggggatgcctacttctctgtgcccctggatgaggacttcaggaagtacactgccttcaccatcccctccatcaacaatgagacccctggcatcaggtaccagtacaatgtgctgccccagggctggaagggctcccctgccatcttccagtcctccatgaccaagatcctggagcccttcaggaagcagaaccctgacattgtgatctaccagtacatggatgacctgtatgtgggctctgacctggagattgggcagcacaggaccaagattgaggagctgaggcagcacctgctgaggtggggcctgaccacccctgacaagaagcaccagaaggagccccccttcctgtggatgggctatgagctgcaccccgacaagtggactgtgcagcccattgtgctgcctgagaaggactcctggactgtgaatgacatccagaagctggtgggcaagctgaactgggcctcccaaatctaccctggcatcaaggtgaggcagctgtgcaagctgctgaggggcaccaaggccctgactgaggtgatccccctgactgaggaggctgagctggagctggctgagaacagggagatcctgaaggagcctgtgcatggggtgtactatgacccctccaaggacctgattgctgagatccagaagcagggccagggccagtggacctaccaaatctaccaggagcccttcaagaacctgaagactggcaagtatgccaggatgaggggggcccacaccaatgatgtgaagcagctgactgaggctgtgcagaagatcaccactgagtccattgtgatctggggcaagacccccaagttcaagctgcccatccagaaggagacctgggagacctggtggactgagtactggcaggccacctggatccctgagtgggagtttgtgaacaccccccccctggtgaagctgtggtaccagctggagaaggagcccattgtgggggctgagaccttctatgtggatggggctgccaacagggagaccaagctgggcaaggctggctatgtgaccaacaggggcaggcagaaggtggtgaccctgactgacaccaccaaccagaagactgagctccaggccatctacctggccctccaggactctggcctggaggtgaacattgtgactgactcccagtatgccctgggcatcatccaggcccagcctgatcagtctgagtctgagctggtgaaccagatcattgagcagctgatcaagaaggagaaggtgtacctggcctgggtgcctgcccacaagggcattgggggcaatgagcaggtggacaagctggtgtctgctggcatcaggaaggtgctgttcctggatggcattgacaaggcccaggatgagcatgagaagtaccactccaactggagggctatggcctctgacttcaacctgccccctgtggtggctaaggagattgtggcctcctgtgacaagtgccagctgaagggggaggccatgcatgggcaggtggactgctcccctggcatctggcagctggactgcacccacctggagggcaaggtgatcctggtggctgtgcatgtggcctccggctacattgaggctgaggtgatccctgctgagacaggccaggagactgcctacttcctgctgaagctggctggcaggtggcctgtgaagaccatccacactgacaatggctccaacttcactggggccacagtgagggctgcctgctggtgggctggcatcaagcaggagtttggcatcccctacaacccccagtcccagggggtggtggagtccatgaacaaggagctgaagaagatcattgggcaggtgagggaccaggctgagcacctgaagacagctgtgcagatggctgtgttcatccacaacttcaagaggaaggggggcatcgggggctactccgctggggagaggattgtggacatcattgccacagacatccagaccaaggagctccagaagcagatcaccaagatccagaacttcagggtgtactacagggactccaggaaccccctgtggaagggccctgccaagctgctgtggaagggggagggggctgtggtgatccaggacaactctgacatcaaggtggtgcccaggaggaaggccaagatcatcagggactatggcaagcagatggctggggatgactgtgtggcctccaggcaggatgaggactaaagcccgggcagatct

SEQ ID NO: 6 - protein, 875 aa, Artificial Sequence
  Codon optimized HIV-1 pol fused to an amino terminal localized leader sequence
MDAMKRGLCCVLLLCGAVFVSPSEISAPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTIHTDNGSNFTGATVRAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 7 - DNA, 2650 bp, Artificial Sequence
  Codon optimized DNA encoding human tPA leader fused to the IA-Pol protein
gatcaccatggatgcaatgaagagagggctctgctgtgtgctgctgctgtgtggagcagtcttcgtttcgcccagcgagatctccgcccccatctcccccattgagactgtgcctgtgaagctgaagcctggcatggatggccccaaggtgaagcagtggcccctgactgaggagaagatcaaggccctggtggaaatctgcactgagatggagaaggagggcaaaatctccaagattggccccgagaacccctacaacacccctgtgtttgccatcaagaagaaggactccaccaagtggaggaagctggtggacttcagggagctgaacaagaggacccaggacttctgggaggtgcagctgggcatcccccaccccgctggcctgaagaagaagaagtctgtgactgtgctggctgtgggggatgcctacttctctgtgcccctggatgaggacttcaggaagtacactgccttcaccatcccctccatcaacaatgagacccctggcatcaggtaccagtacaatgtgctgccccagggctggaagggctcccctgccatcttccagtcctccatgaccaagatcctggagcccttcaggaagcagaaccctgacattgtgatctaccagtacatggctgccctgtatgtgggctctgacctggagattgggcagcacaggaccaagattgaggagctgaggcagcacctgctgaggtggggcctgaccacccctgacaagaagcaccagaaggagccccccttcctgtggatgggctatgagctgcaccccgacaagtggactgtgcagcccattgtgctgcctgagaaggactcctggactgtgaatgacatccagaagctggtgggcaagctgaactgggcctcccaaatctaccctggcatcaaggtgaggcagctgtgcaagctgctgaggggcaccaaggccctgactgaggtgatccccctgactgaggaggctgagctggagctggctgagaacagggagatcctgaaggagcctgtgcatggggtgtactatgacccctccaaggacctgattgctgagatccagaagcagggccagggccagtggacctaccaaatctaccaggagcccttcaagaacctgaagactggcaagtatgccaggatgaggggggcccacaccaatgatgtgaagcagctgactgaggctgtgcagaagatcaccactgagtccattgtgatctggggcaagacccccaagttcaagctgcccatccagaaggagacctgggagacctggtggactgagtactggcaggccacctggatccctgagtgggagtttgtgaacaccccccccctggtgaagctgtggtaccagctggagaaggagcccattgtgggggctgagaccttctatgtggctggggctgccaacagggagaccaagctgggcaaggctggctatgtgaccaacaggggcaggcagaaggtggtgaccctgactgacaccaccaaccagaagactgccctccaggccatctacctggccctccaggactctggcctggaggtgaacattgtgactgcctcccagtatgccctgggcatcatccaggcccagcctgatcagtctgagtctgagctggtgaaccagatcattgagcagctgatcaagaaggagaaggtgtacctggcctgggtgcctgcccacaagggcattgggggcaatgagcaggtggacaagctggtgtctgctggcatcaggaaggtgctgttcctggatggcattgacaaggcccaggatgagcatgagaagtaccactccaactggagggctatggcctctgacttcaacctgccccctgtggtggctaaggagattgtggcctcctgtgacaagtgccagctgaagggggaggccatgcatgggcaggtggactgctcccctggcatctggcagctggcctgcacccacctggagggcaaggtgatcctggtggctgtgcatgtggcctccggctacattgaggctgaggtgatccctgctgagacaggccaggagactgcctacttcctgctgaagctggctggcaggtggcctgtgaagaccatccacactgccaatggctccaacttcactggggccacagtgagggctgcctgctggtgggctggcatcaagcaggagtttggcatcccctacaacccccagtcccagggggtggtggcctccatgaacaaggagctgaagaagatcattgggcaggtgagggaccaggctgagcacctgaagacagctgtgcagatggctgtgttcatccacaacttcaagaggaaggggggcatcgggggctactccgctggggagaggattgtggacatcattgccacagacatccagaccaaggagctccagaagcagatcaccaagatccagaacttcagggtgtactacagggactccaggaaccccctgtggaagggccctgccaagctgctgtggaagggggagggggctgtggtgatccaggacaactctgacatcaaggtggtgcccaggaggaaggccaagatcatcagggactatggcaagcagatggctggggatgactgtgtggcctccaggcaggatgaggactaaagcccgggcagatct

SEQ ID NO: 8 - protein, 875 aa, Artificial Sequence
  Codon optimized human tPA leader fused to the IA-Pol protein
MDAMKRGLCCVLLLCGAVFVSPSEISAPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLAVGDAYFSVPLDEDFRKYTAFTIPSINNETPGIRYQYNVLPQGWKGSPAIFQSSMTKILEPFRKQNPDIVIYQYMAALYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVAGAANRETKLGKAGYVTNRGRQKVVTLTDTTNQKTALQAIYLALQDSGLEVNIVTASQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLACTHLEGKVILVAVHVASGYIEAEVIPAETGQETAYFLLKLAGRWPVKTIHTANGSNFTGATVRAACWWAGIKQEFGIPYNPQSQGVVASMNKELKKIIGQVRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 9 - DNA, 671 bp, Artificial Sequence
  Codon optimized DNA encoding  HIV-1 nef from the HIV-1 jfrl isolate
gatctgccaccatgggcggcaagtggtccaagaggtccgtgcccggctggtccaccgtgagggagaggatgaggagggccgagcccgccgccgacagggtgaggaggaccgagcccgccgccgtgggcgtgggcgccgtgtccagggacctggagaagcacggcgccatcacctcctccaacaccgccgccaccaacgccgactgcgcctggctggaggcccaggaggacgaggaggtgggcttccccgtgaggccccaggtgcccctgaggcccatgacctacaagggcgccgtggacctgtcccacttcctgaaggagaagggcggcctggagggcctgatccactcccagaagaggcaggacatcctggacctgtgggtgtaccacacccagggctacttccccgactggcagaactacacccccggccccggcatcaggttccccctgaccttcggctggtgcttcaagctggtgcccgtggagcccgagaaggtggaggaggccaacgagggcgagaacaactgcctgctgcaccccatgtcccagcacggcatcgaggaccccgagaaggaggtgctggagtggaggttcgactccaagctggccttccaccacgtggccagggagctgcaccccgagtactacaaggactgctaaagcccgggc

SEQ ID NO: 10 - protein, 216 aa, Artificial Sequence
  Codon optimized HIV-1 nef from the HIV-1 jfrl isolate
MGGKWSKRSVPGWSTVRERMRRAEPAADRVRRTEPAAVGVGAVSRDLEKHGAITSSNTAATNADCAWLEAQEDEEVGFPVRPQVPLRPMTYKGAVDLSHFLKEKGGLEGLIHSQKRQDILDLWVYHTQGYFPDWQNYTPGPGIRFPLTFGWCFKLVPVEPEKVEEANEGENNCLLHPMSQHGIEDPEKEVLEWRFDSKLAFHHVARELHPEYYKDC

SEQ ID NO: 11 - DNA, 719 bp, Artificial Sequence
  Codon optimized DNA encoding human plasminogen activator (tPA) leader fused with the NH2-terminus of HIV-1 nef
catggatgcaatgaagagagggctctgctgtgtgctgctgctgtgtggagcagtcttcgtttcgcccagcgagatctcctccaagaggtccgtgcccggctggtccaccgtgagggagaggatgaggagggccgagcccgccgccgacagggtgaggaggaccgagcccgccgccgtgggcgtgggcgccgtgtccagggacctggagaagcacggcgccatcacctcctccaacaccgccgccaccaacgccgactgcgcctggctggaggcccaggaggacgaggaggtgggcttccccgtgaggccccaggtgcccctgaggcccatgacctacaagggcgccgtggacctgtcccacttcctgaaggagaagggcggcctggagggcctgatccactcccagaagaggcaggacatcctggacctgtgggtgtaccacacccagggctacttccccgactggcagaactacacccccggccccggcatcaggttccccctgaccttcggctggtgcttcaagctggtgcccgtggagcccgagaaggtggaggaggccaacgagggcgagaacaactgcctgctgcaccccatgtcccagcacggcatcgaggaccccgagaaggaggtgctggagtggaggttcgactccaagctggccttccaccacgtggccagggagctgcaccccgagtactacaaggactgctaaagcc

SEQ ID NO: 12 - protein, 237 aa, Artificial Sequence
  Codon optimized DNA for human plasminogen activator (tPA) leader fused with the NH2-terminus of HIV-1 nef
MDAMKRGLCCVLLLCGAVFVSPSEISSKRSVPGWSTVRERMRRAEPAADRVRRTEPAAVGVGAVSRDLEKHGAITSSNTAATNADCAWLEAQEDEEVGFPVRPQVPLRPMTYKGAVDLSHFLKEKGGLEGLIHSQKRQDILDLWVYHTQGYFPDWQNYTPGPGIRFPLTFGWCFKLVPVEPEKVEEANEGENNCLLHPMSQHGIEDPEKEVLEWRFDSKLAFHHVARELHPEYYKDC

SEQ ID NO: 13 - DNA, 671 bp, Artificial Sequence
  DNA encoding optimized modified HIV-1 nef
gatctgccaccatggccggcaagtggtccaagaggtccgtgcccggctggtccaccgtgagggagaggatgaggagggccgagcccgccgccgacagggtgaggaggaccgagcccgccgccgtgggcgtgggcgccgtgtccagggacctggagaagcacggcgccatcacctcctccaacaccgccgccaccaacgccgactgcgcctggctggaggcccaggaggacgaggaggtgggcttccccgtgaggccccaggtgcccctgaggcccatgacctacaagggcgccgtggacctgtcccacttcctgaaggagaagggcggcctggagggcctgatccactcccagaagaggcaggacatcctggacctgtgggtgtaccacacccagggctacttccccgactggcagaactacacccccggccccggcatcaggttccccctgaccttcggctggtgcttcaagctggtgcccgtggagcccgagaaggtggaggaggccaacgagggcgagaacaactgcgccgcccaccccatgtcccagcacggcatcgaggaccccgagaaggaggtgctggagtggaggttcgactccaagctggccttccaccacgtggccagggagctgcaccccgagtactacaaggactgctaaagcccgggc

SEQ ID NO: 14 - protein, 217 aa, Artificial Sequence
  Codon optimized modified HIV-1 nef
MAGKWSKRSVPGWSTVRERMRRAEPAADRVRRTEPAAVGVGAVSRDLEKHGAITSSNTAATNADCAWLEAQEDEEVGFPVRPQVPLRPMTYKGAVDLSHFLKEKGGLEGLIHSQKRQDILDLWVYHTQGYFPDWQNYTPGPGIRFPLTFGWCFKLVPVEPEKVEEANEGENNCAAHPMSQHGIEDPEKEVLEWRFDSKLAFHHVARELHPEYYKDCS

SEQ ID NO: 15 - DNA, 720 bp, Artificial Sequence
  Codon optimized DNA encoding modified HIV-1nef fused to a leader sequence
catggatgcaatgaagagagggctctgctgtgtgctgctgctgtgtggagcagtcttcgtttcgcccagcgagatctcctccaagaggtccgtgcccggctggtccaccgtgagggagaggatgaggagggccgagcccgccgccgacagggtgaggaggaccgagcccgccgccgtgggcgtgggcgccgtgtccagggacctggagaagcacggcgccatcacctcctccaacaccgccgccaccaacgccgactgcgcctggctggaggcccaggaggacgaggaggtgggcttccccgtgaggccccaggtgcccctgaggcccatgacctacaagggcgccgtggacctgtcccacttcctgaaggagaagggcggcctggagggcctgatccactcccagaagaggcaggacatcctggacctgtgggtgtaccacacccagggctacttccccgactggcagaactacacccccggccccggcatcaggttccccctgaccttcggctggtgcttcaagctggtgcccgtggagcccgagaaggtggaggaggccaacgagggcgagaacaactgcgccgcccaccccatgtcccagcacggcatcgaggaccccgagaaggaggtgctggagtggaggttcgactccaagctggccttccaccacgtggccagggagctgcaccccgagtactacaaggactgctaaagccc

SEQ ID NO: 16 - protein, 237 aa, Artificial Sequence
  Codon optimized modified HIV-1 nef fused to a leader sequence
MDAMKRGLCCVLLLCGAVFVSPSEISSKRSVPGWSTVRERMRRAEPAADRVRRTEPAAVGVGAVSRDLEKHGAITSSNTAATNADCAWLEAQEDEEVGFPVRPQVPLRPMTYKGAVDLSHFLKEKGGLEGLIHSQKRQDILDLWVYHTQGYFPDWQNYTPGPGIRFPLTFGWCFKLVPVEPEKVEEANEGENNCAAHPMSQHGIEDPEKEVLEWRFDSKLAFHHVARELHPEYYKDC

SEQ ID NO: 17 - protein, 27 aa, Artificial Sequence
  Amino acid sequence of human tPA leader
MDAMKRGLCCVLLLCGAVFVSPSEISS

SEQ ID NO: 18 - DNA, 49 bp, Artificial Sequence
  Short synthetic polyA signal (SPA)
aataaaagatctttattttcattagatctgtgtgttggttttttgtgtg

SEQ ID NO: 19 - DNA, 651 bp, Human Immunodeficiency Virus
atgggtggcaagtggtcaaaacgtagtgtgcctggatggtctactgtaagggaaagaatgagacgagctgagccagcagcagatagggtgagacgaactgagccagcagcagtaggggtgggagcagtatctcgagacctggaaaaacatggagcaatcacaagtagcaatacagcagctaccaatgctgattgtgcctggctagaagcacaagaggatgaggaagtgggttttccagtcagacctcaggtacctttaagaccaatgacttacaagggagctgtagatcttagccactttttaaaagaaaaggggggactggaagggctaattcactcacagaaaagacaagatatccttgatctgtgggtctaccacacacaaggctacttccctgattggcagaactacacaccagggccaggaatcagatttccattgacctttggatggtgcttcaagctagtaccagttgagccagaaaaggtagaagaggccaatgaaggagagaacaactgcttgttacaccctatgagccagcatgggatagaggacccggagaaggaagtgttagagtggaggtttgacagcaagctagcatttcatcacgtggcccgagagctgcatccggagtactacaaggactgctga

SEQ ID NO: 20 - DNA, 35 bp, Artificial Sequence
  PCR primer
ataagaatgcggccgccatatactgagtcattagg

SEQ ID NO: 21 - DNA, 31 bp, Artificial Sequence
  PCR primer
aaggaagatctaccgacgctggtcgcgcctc

SEQ ID NO: 22 - DNA, 34 bp, Artificial Sequence
  PCR primer
ataagaatggcgcgccatatactgagtcattagg

SEQ ID NO: 23 - DNA, 31 bp, Artificial Sequence; identical to SEQ ID NO: 21
  PCR primer

SEQ ID NO: 24 - DNA, 30 bp, Artificial Sequence
  PCR primer
attggatccatggatgcaatgaagagaggg

SEQ ID NO: 25 - DNA, 37474 bp, Artificial Sequence
  DNA encoding pMRKAd5 HIV-1 gag, coding
ttcttaattaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattataggcggccgcgatccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgagatctaccatgggtgctagggcttctgtgctgtctggtggtgagctggacaagtgggagaagatcaggctgaggcctggtggcaagaagaagtacaagctaaagcacattgtgtgggcctccagggagctggagaggtttgctgtgaaccctggcctgctggagacctctgaggggtgcaggcagatcctgggccagctccagccctccctgcaaacaggctctgaggagctgaggtccctgtacaacacagtggctaccctgtactgtgtgcaccagaagattgatgtgaaggacaccaaggaggccctggagaagattgaggaggagcagaacaagtccaagaagaaggcccagcaggctgctgctggcacaggcaactccagccaggtgtcccagaactaccccattgtgcagaacctccagggccagatggtgcaccaggccatctccccccggaccctgaatgcctgggtgaaggtggtggaggagaaggccttctcccctgaggtgatccccatgttctctgccctgtctgagggtgccaccccccaggacctgaacaccatgctgaacacagtggggggccatcaggctgccatgcagatgctgaaggagaccatcaatgaggaggctgctgagtgggacaggctgcatcctgtgcacgctggccccattgcccccggccagatgagggagcccaggggctctgacattgctggcaccacctccaccctccaggagcagattggctggatgaccaacaacccccccatccctgtgggggaaatctacaagaggtggatcatcctgggcctgaacaagattgtgaggatgtactcccccacctccatcctggacatcaggcagggccccaaggagcccttcagggactatgtggacaggttctacaagaccctgagggctgagcaggcctcccaggaggtgaagaactggatgacagagaccctgctggtgcagaatgccaaccctgactgcaagaccatcctgaaggccctgggccctgctgccaccctggaggagatgatgacagcctgccagggggtggggggccctggtcacaaggccagggtgctggctgaggccatgtcccaggtgaccaactccgccaccatcatgatgcagaggggcaacttcaggaaccagaggaagacagtgaagtgcttcaactgtggcaaggtgggccacattgccaagaactgtagggcccccaggaagaagggctgctggaagtgtggcaaggagggccaccagatgaaggactgcaatgagaggcaggccaacttcctgggcaaaatctggccctcccacaagggcaggcctggcaacttcctccagtccaggcctgagcccacagcccctcccgaggagtccttcaggtttggggaggagaagaccacccccagccagaagcaggagcccattgacaaggagctgtaccccctggcctccctgaggtccctgtttggcaacgacccctcctcccagtaaaataaagcccgggcagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccgatcggcgcgccgtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaaacagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaacgcaatagatgccatgaaccacccaactttccccgcgcccgctatgcttccactgcaacaagttgttgccggcggctttgtcccagccaatcagcctcgcccaccttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctcgtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctttaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaagaattcggatctgcgacgcgaggctggatggccttccccattatgattcttctcgcttccggcggcatcgggatgcccgcgttgcaggccatgctgtccaggcaggtagatgacgaccatcagggacagcttcaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggatccgaattcttaat

SEQ ID NO: 26 - DNA, 37474 bp, Artificial Sequence
  DNA encoding pMRKAd5 HIV-1 gag, noncoding
aagaattaattgtagtagttattatatggaataaaacctaacttcggttatactattactcccccacctcaaacactgcaccgcgccccgcacccttgccccgcccactgcatcatcacaccgccttcacactacaacgttcacaccgccttgtgtacattcgctgcctacaccgttttcactgcaaaaaccacacgcggccacatgtgtccttcactgttaaaagcgcgccaaaatccgcctacaacatcatttaaacccgcattggctcattctaaaccggtaaaagcgcccttttgacttattctccttcactttagacttattaaaacacaatgagtatcgcgcattataaacagatcccggcgcccctgaaactggcaaatgcacctctgagcgggtccacaaaaagagtccacaaaaggcgcaaggcccagtttcaaccgcaaaataataatatccgccggcgctaggtaacgtatgcaacataggtatagtattatacatgtaaatataaccgagtacaggttgtaatggcggtacaactgtaactaataactgatcaataattatcattagttaatgccccagtaatcaagtatcgggtatatacctcaaggcgcaatgtattgaatgccatttaccgggcggaccgactggcgggttgctgggggcgggtaactgcagttattactgcatacaagggtatcattgcggttatccctgaaaggtaactgcagttacccacctcataaatgccatttgacgggtgaaccgtcatgtagttcacatagtatacggttcatgcgggggataactgcagttactgccatttaccgggcggaccgtaatacgggtcatgtactggaataccctgaaaggatgaaccgtcatgtagatgcataatcagtagcgataatggtaccactacgccaaaaccgtcatgtagttacccgcacctatcgccaaactgagtgcccctaaaggttcagaggtggggtaactgcagttaccctcaaacaaaaccgtggttttagttgccctgaaaggttttacagcattgttgaggcggggtaactgcgtttacccgccatccgcacatgccaccctccagatatattcgtctcgagcaaatcacttggcagtctagcggacctctgcggtaggtgcgacaaaactggaggtatcttctgtggccctggctaggtcggaggcgccggcccttgccacgtaaccttgcgcctaaggggcacggttctcactctagatggtacccacgatcccgaagacacgacagaccaccactcgacctgttcaccctcttctagtccgactccggaccaccgttcttcttcatgttcgatttcgtgtaacacacccggaggtccctcgacctctccaaacgacacttgggaccggacgacctctggagactccccacgtccgtctaggacccggtcgaggtcgggagggacgtttgtccgagactcctcgactccagggacatgttgtgtcaccgatgggacatgacacacgtggtcttctaactacacttcctgtggttcctccgggacctcttctaactcctcctcgtcttgttcaggttcttcttccgggtcgtccgacgacgaccgtgtccgttgaggtcggtccacagggtcttgatggggtaacacgtcttggaggtcccggtctaccacgtggtccggtagaggggggcctgggacttacggacccacttccaccacctcctcttccggaagaggggactccactaggggtacaagagacgggacagactcccacggtggggggtcctggacttgtggtacgacttgtgtcaccccccggtagtccgacggtacgtctacgacttcctctggtagttactcctccgacgactcaccctgtccgacgtaggacacgtgcgaccggggtaacgggggccggtctactccctcgggtccccgagactgtaacgaccgtggtggaggtgggaggtcctcgtctaaccgacctactggttgttgggggggtagggacaccccctttagatgttctccacctagtaggacccggacttgttctaacactcctacatgagggggtggaggtaggacctgtagtccgtcccggggttcctcgggaagtccctgatacacctgtccaagatgttctgggactcccgactcgtccggagggtcctccacttcttgacctactgtctctgggacgaccacgtcttacggttgggactgacgttctggtaggacttccgggacccgggacgacggtgggacctcctctactactgtcggacggtcccccaccccccgggaccagtgttccggtcccacgaccgactccggtacagggtccactggttgaggcggtggtagtactacgtctccccgttgaagtccttggtctccttctgtcacttcacgaagttgacaccgttccacccggtgtaacggttcttgacatcccgggggtccttcttcccgacgaccttcacaccgttcctcccggtggtctacttcctgacgttactctccgtccggttgaaggacccgttttagaccgggagggtgttcccgtccggaccgttgaaggaggtcaggtccggactcgggtgtcggggagggctcctcaggaagtccaaacccctcctcttctggtgggggtcggtcttcgtcctcgggtaactgttcctcgacatgggggaccggagggactccagggacaaaccgttgctggggaggagggtcattttatttcgggcccgtctagacgacacggaagatcaacggtcggtagacaacaaacggggagggggcacggaaggaactgggaccttccacggtgagggtgacaggaaaggattattttactcctttaacgtagcgtaacagactcatccacagtaagataagaccccccaccccaccccgtcctgtcgttccccctcctaacccttctgttatcgtccgtacgacccctacgccacccgagataccggctagccgcgcggcatgactttacacacccgcaccgaattcccaccctttcttatatattccacccccagaatacatcaaaacatagacaaaacgtcgtcggcggcggcggtactcgtggttgagcaaactaccttcgtaacactcgagtataaactgttgcgcgtacgggggtacccggccccacgcagtcttacactacccgaggtcgtaactaccagcggggcaggacgggcgtttgagatgatggaactggatgctctggcacagaccttgcggcaacctctgacgtcggaggcggcggcgaagtcggcgacgtcggtggcgggcgccctaacactgactgaaacgaaaggactcgggcgaacgtttgtcacgtcgaagggcaagtaggcgggcgctactgttcaactgccgagaaaaccgtgttaacctaagaaactgggcccttgaattacagcaaagagtcgtcgacaacctagacgcggtcgtccaaagacgggacttccgaaggaggggagggttacgccaaattttgtatttattttttggtctgagacaaacctaaacctagttcgttcacagaacgacagaaataaatccccaaaacgcgcgcgccatccgggccctggtcgccagagccagcaactcccaggacacataaaaaaggtcctgcaccatttccactgagacctacaagtctatgtacccgtattcgggcagagaccccacctccatcgtggtgacgtctcgaagtacgacgccccaccacaacatctactaggtcagcatcgtcctcgcgacccgcaccacggatttttacagaaagtcatcgttcgactaacggtccccgtccgggaaccacattcacaaatgtttcgccaattcgaccctacccacgtatgcacccctatactctacgtagaacctgacataaaaatccaaccgatacaagggtcggtatagggaggcccctaagtacaacacgtcttggtggtcgtgtcacataggccacgtgaaccctttaaacagtacatcgaatcttcctttacgcaccttcttgaacctctgcgggaacactggaggttctaaaaggtacgtaagcaggtattactaccgttacccgggtgcccgccgccggacccgcttctataaagaccctagtgattgcagtatcaacacaaggtcctactctagcagtatccggtaaaaatgtttcgcgcccgcctcccacggtctgacgccatattaccaaggtaggccgggtccccgcatcaatgggagtgtctaaacgtaaagggtgcgaaactcaagtctacccccctagtacagatggacgccccgctacttcttttgccaaaggccccatcccctctagtcgacccttctttcgtccaaggactcgtcgacgctgaatggcgtcggccacccgggcatttagtgtggataatggccgacgttgaccatcaattctctcgacgtcgacggcagtagggactcgtccccccggtgaagcaattcgtacagggactgagcgtacaaaagggactggtttaggcggtcttccgcgagcggcgggtcgctatcgtcaagaacgttccttcgtttcaaaaagttgccaaactctggcaggcggcatccgtacgaaaactcgcaaactggttcgtcaaggtccgccagggtgtcgagccagtggacgagatgccgtagagctaggtcgtatagaggagcaaagcgcccaaccccgccgaaagcgacatgccgtcatcagccacgagcaggtctgcccggtcccagtacagaaaggtgcccgcgtcccaggagcagtcgcatcagacccagtgccacttccccacgcgaggcccgacgcgcgaccggtcccacgcgaactccgaccaggacgaccacgacttcgcgacggccagaagcgggacgcgcagccggtccatcgtaaactggtaccacagtatcaggtcggggaggcgccgcaccgggaaccgcgcgtcgaacgggaacctcctccgcggcgtgctccccgtcacgtctgaaaactcccgcatctcgaacccgcgctctttatggctaaggcccctcatccgtaggcgcggcgtccggggcgtctgccagagcgtaaggtgctcggtccactcgagaccggcaagccccagtttttggtccaaagggggtacgaaaaactacgcaaagaatggagaccaaaggtactcggccacaggtgcgagccactgcttttccgacaggcacaggggcatatgtctgaactctccggacaggagctcgccacaaggcgccaggaggagcatatctttgagcctggtgagactctgtttccgagcgcaggtccggtcgtgcttcctccgattcaccctccccatcgccagcaacaggtgatcccccaggtgagcgaggtcccacacttctgtgtacagcgggagaagccgtagttccttccactaaccaaacatccacatccggtgcactggcccacaaggacttccccccgatattttcccccacccccgcgcaagcaggagtgagagaaggcgtagcgacagacgctcccggtcgacaaccccactcatgagggagacttttcgcccgtactgaagacgcgattctaacagtcaaaggtttttgctcctcctaaactataagtggaccgggcgccactacggaaactcccaccggcgtaggtagaccagtcttttctgttagaaaaacaacagttcgaaccaccgtttgctgggcatctcccgcaacctgtcgttgaaccgctacctcgcgtcccaaaccaaaaacagcgctagccgcgcgaggaaccggcgctacaaatcgacgtgcataagcgcgcgttgcgtggcggtaagccctttctgccaccacgcgagcagcccgtggtccacgtgcgcggttggcgccaacacgtcccactgttccagttgcgaccaccgatggagaggcgcatccgcgagcaaccaggtcgtctccgccggcgggaacgcgctcgtcttaccgccatcccccagatcgacgcagagcaggccccccagacgcaggtgccatttctggggcccgtcgtccgcgcgcagcttcatcagatagaacgtaggaacgttcagatcgcggacgacggtacgcgcccgccgttcgcgcgcgagcatacccaactcaccccctggggtaccgtaccccacccactcgcgcctccgcatgtacggcgtttacagcatttgcatctccccgagagactcataaggttctatacatcccatcgtagaaggtggcgcctacgaccgcgcgtgcattagcatatcaagcacgctccctcgctcctccagccctggctccaacgatgcccgcccgacgagacgagccttctgatagacggacttctaccgtacactcaacctactataccaacctgcgaccttctgcaacttcgaccgcagacactctggatggcgcagtgcgtgcttcctccgcatcctcagcgcgtcgaacaactggtcgagccgccactggacgtgcagatcccgcgtcatcaggtcccaaaggaactactacagtatgaataggacagggaaaaaaaaggtgtcgagcgccaactcctgtttgagaagcgccagaaaggtcatgagaacctagcctttgggcagccggaggcttgccattctcggatcgtacatcttgaccaactgccggaccatccgcgtcgtagggaaaagatgcccatcgcgcatacggacgcgccggaaggcctcgctccacacccactcgcgtttccacagggactggtactgaaactccatgaccataaacttcagtcacagcagcgtaggcgggacgagggtctcgtttttcaggcacgcgaaaaaccttgcgcctaaaccgtcccgcttccactgtagcaacttctcatagaaagggcgcgctccgtatttcaacgcacactacgccttcccagggccgtggagccttgccaacaattaatggacccgccgctcgtgctagagcagtttcggcaactacaacaccgggtgttacatttcaaggttcttcgcgccctacgggaactaccttccgttaaaaaattcaaggagcatccactcgagaagtcccctcgactcgggcacgagactttcccgggtcagacgttctactcccaaccttcgctgcttactcgaggtgtccagtgcccggtaatcgtaaacgtccaccagcgctttccaggatttgaccgctggataccggtaaaaaagaccccactacgtcatcttccattcgcccagaacaagggtcgccagggtaggttccaagcgccgatccagagcgcgccgtcagtgatctccgagtagaggcggcttgaagtactggtcgtacttcccgtgctcgacgaagggtttccgggggtaggttcatatccagagatgtagcatccactgtttctctgcgagccacgctcctacgctcggctagcccttcttgacctagagggcggtggttaacctcctcaccgataactacaccactttcatcttcagggacgctgcccggcttgtgagcacgaccgaaaacatttttgcacgcgtcatgaccgtcgccacgtgcccgacatgtaggacgtgctccaactggactgctggcgcgtgttccttcgtctcacccttaaactcggggagcggaccgcccaaaccgaccaccagaagatgaagccgacgaacaggaactggcagaccgacgagctcccctcaatgccacctagcctggtggtgcggcgcgctcgggtttcaggtctacaggcgcgcgccgccagcctcgaactactgttgtagcgcgtctaccctcgacaggtaccagacctcgagggcgccgcagtccagtccgccctcgaggacgtccaaatggagcgtatctgcccagtcccgcgcccgatctaggtccactatggattaaaggtccccgaccaaccaccgccgcagctaccgaacgttctccggcgtaggggcgccgcgctgatgccatggcgcgccgcccgccacccggcgcccccacaggaacctactacgtagattttcgccactgcgcccgctcgggggcctccatcccccccgaggcctgggcggccctctcccccgtccccgtgcagccgcggcgcgcgcccgtcctcgaccacgacgcgcgcatccaacgaccgcttgcgctgctgcgccgccaactagaggacttagaccgcggagacgcacttctgctgcccgggccactcgaacttggactttctctcaagctgtcttagttaaagccacagcaactgccgccggaccgcgttttagaggacgtgcagaggactcaacagaactatccgctagagccggtacttgacgagctagagaaggaggacctctagaggcgcaggccgagcgaggtgccaccgccgctccagcaacctttacgcccggtactcgacgctcttccgcaactccggagggagcaaggtctgcgccgacatctggtgcgggggaagccgtagcgcccgcgcgtactggtggacgcgctctaactcgaggtgcacggcccgcttctgccgcatcaaagcgtccgcgactttctccatcaactcccaccaccgccacacaagacggtgcttcttcatgtattgggtcgcagcgttgcacctaagcaactatagggggttccggagttccgcgaggtaccggagcatcttcaggtgccgcttcaactttttgaccctcaacgcgcggctgtgccaattgaggaggaggtcttctgcctactcgagccgctgtcacagcgcgtggagcgcgagtttccgatgtccccggagaagaagaagaagttagaggagaaggtattcccggaggggaagaagaagaagaccgccgccaccccctcccccctgtgccgccgctgctgccgcgtggccctccgccagctgtttcgcgagctagtagaggggcgccgctgccgcgtaccagagccactgccgcgccggcaagagcgcccccgcgtcaaccttctgcggcgggcagtacagggccaatacccaaccgccccccgacggtacgccgtccctatgccgcgattgctacgtagagttgttaacaacacatccatgaggcggcggctccctggactcgctcaggcgtagctggcctagccttttggagagctctttccgcagattggtcagtgtcagcgttccatccgactcgtggcaccgcccgccgtcgcccgccgccagccccaacaaagaccgcctccacgacgactactacattaatttcatccgccagaactctgccgcctaccagctgtcttcgtggtacaggaacccaggccggacgacttacgcgtccgccagccggtacggggtccgaagcaaaactgtagccgcgtccagaaacatcatcagaacgtactcggaaagatggccgtgaagaagaagaggaaggagaacaggacgtagagaacgtagatagcgacgccgccgccgcctcaaaccggcatccaccgcgggagaaggagggtacgcacactggggcttcggggagtagccgacttcgtcccgatccagccgctgttgcgcgagccgattataccggacgacgtggacgcactcccatctgaccttcagtaggtacaggtgtttcgccaccatacgcgggcacaactaccacattcacgtcaaccggtattgcctggtcaattgccagaccactgggccgacgctctcgagccacatggactctgcgctcattcgggagctcagtttatgcatcagcaacgttcaggcgtggtccatgaccatagggtggtttttcacgccgccgccgaccgccatctccccggtcgcatcccaccggccccgaggcccccgctctagaaggttgtattccgctactataggcatctacatggacctgtaggtccactacggccgccgccaccacctccgcgcgcctttcagcgcctgcgccaaggtctacaacgcgtcgccgtttttcacgaggtaccagccctgcgagaccggccagtccgcgcgcgttagcaactgcgagatctggcacgttttcctctcggacattcgcccgtgagaaggcaccagaccacctatttaagcgttcccatagtaccgcctgctggccccaagctcggggcataggccggcaggcggcactaggtacgccaatggcgggcgcacagcttgggtccacacgctgcagtctgttgccccctcacgaggaaaaccgaaggaaggtccgcgccgccgacgacgcgatcgaaaaaaccggtgaccggcgcgcgtcgcattcgccaatccgacctttcgctttcgtaattcaccgagcgagggacatcggcctcccaataaaaggttcccaactcagcgccctgggggccaagctcagagcctggccggcctgacgccgcttgcccccaaacggaggggcagtacgttctggggcgaacgtttaaggaggcctttgtccctgctcggggaaaaaacgaaaagggtctacgtaggccacgacgccgtctacgcggggggaggagtcgtcgccgttctcgttctcgtcgccgtctgtacgtcccgtgggaggggaggaggatggcgcagtcctccccgctgtaggcgccaactgcgccgtcgtctaccactaatgcttgggggcgccgcggcccgggccgtgatggacctgaacctcctcccgctcccggaccgcgccgatcctcgcgggagaggactcgccgtgggttcccacgtcgacttcgcactatgcgcactccgcatgcacggcgccgtcttggacaaagcgctggcgctccctctcctcgggctcctctacgccctagctttcaaggtgcgtcccgcgctcgacgccgtaccggacttagcgctcgccaacgacgcgctcctcctgaaactcgggctgcgcgcttggccctaatcagggcgcgcgcgtgtgcaccgccggcggctggaccattggcgtatgctcgtctgccacttggtcctctaattgaaagttttttcgaaattgttggtgcacgcatgcgaacaccgcgcgctcctccaccgatatcctgactacgtagacaccctgaaacattcgcgcgacctcgttttgggtttatcgttcggcgagtaccgcgtcgacaaggaatatcacgtcgtgtcgtccctgttgctccgtaagtccctacgcgacgatttgtatcatctcgggctcccggcgaccgacgagctaaactatttgtaggacgtctcgtatcaccacgtcctcgcgtcgaactcggaccgactgttccaccggcggtagttgataaggtacgaatcggacccgttcaaaatgcgggcgttctatatggtatggggaatgcaagggtatctgttcctccatttctagctccccaagatgtacgcgtaccgcgacttccacgaatggaactcgctgctggacccgcaaatagcgttgctcgcgtaggtgttccggcactcgcactcggccgccgcgctcgagtcgctggcgctcgactacgtgtcggacgtttcccgggaccgaccgtgcccgtcgccgctatctctccggctcaggatgaaactgcgcccgcgactggacgcgacccggggttcggctgcgcgggacctccgtcgaccccggcctggacccgaccgccaccgtgggcgcgcgcgaccgttgcagccgccgcacctccttatactgctcctgctactcatgctcggtctcctgccgctcatgattcgccactacaaagactagtctactacgttctgcgttgcctgggccgccacgcccgccgcgacgtctcggtcggcaggccggaattgaggtgcctgctgaccgcggtccagtacctggcgtagtacagcgactgacgcgcgttaggactgcgcaaggccgtcgtcggcgtccggttggccgagaggcgttaagaccttcgccaccagggccgcgcgcgtttggggtgcgtgctcttccacgaccgctagcatttgcgcgaccggcttttgtcccggtaggccgggctgctccggccggaccagatgctgcgcgacgaagtcgcgcaccgagcaatgttgtcgccgttgcacgtctggttggacctggccgaccaccccctacacgcgctccggcaccgcgtcgcactcgcgcgcgtcgtcgtcccgttggacccgaggtaccaacgtgatttgcggaaggactcatgtgtcgggcggttgcacggcgcccctgtcctcctgatgtggttgaaacactcgcgtgacgccgattaccactgactctgtggcgtttcactccacatggtcagacccggtctgataaaaaaggtctggtcatctgttccggacgtctggcatttggactcggtccgaaagtttttgaacgtccccgacaccccccacgcccgagggtgtccgctggcgcgctggcacagatcgaacgactgcgggttgagcgcggacaacgacgacgattatcgcgggaagtgcctgtcaccgtcgcacagggccctgtgtatggatccagtgaacgactgtgacatggcgctccggtatccagtccgcgtacacctgctcgtatgaaaggtcctctaatgttcacagtcggcgcgcgaccccgtcctcctgtgcccgtcggacctccgttgggatttgatggacgactggttggccgccgtcttctaggggagcaacgtgtcaaatttgtcgctcctcctcgcgtaaaacgcgatgcacgtcgtctcgcactcggaattggactacgcgctgccccattgcgggtcgcaccgcgacctgtactggcgcgcgttgtaccttggcccgtacatacggagtttggccggcaaatagttggcggattacctgatgaacgtagcgcgccggcggcacttggggctcataaagtggttacggtagaacttgggcgtgaccgatggcgggggaccaaagatgtggccccctaagctccacgggctcccattgctacctaaggagaccctgctgtatctgctgtcgcacaaaaggggcgttggcgtctgggacgatctcaacgttgtcgcgctcgtccgtctccgccgcgacgctttcctttcgaaggcgtccggttcgtcgaacaggctagatccgcgacgccggggcgccagtctacgatcatcgggtaaaggttcgaactatcccagagaatggtcgtgagcgtggtgggcgggcgcggacgacccgctcctcctcatggatttgttgagcgacgacgtcggcgtcgcgctttttttggacggaggccgtaaagggttgttgccctatctctcggatcacctgttctactcatctaccttctgcatgcgcgtcctcgtgtccctgcacggtccgggcgcgggcgggtgggcagcagtttccgtgctggcagtcgccccagaccacaccctcctgctactgagccgtctgctgtcgtcgcaggacctaaaccctccctcaccgttgggcaaacgcgtggaagcggggtccgacccctcttacaaaatttttttttttttcgtactacgttttattttttgagtggttccggtaccgtggctcgcaaccaaaagaacataaggggaatcatacgccgcgcgccgctacatactccttccaggaggagggaggatgctctcacaccactcgcgccgcggtcaccgccgccgcgacccaagagggaagctacgaggggacctgggcggcaaacacggaggcgccatggacgccggatggcccccctctttgtcgtaggcaatgagactcaaccgtggggataagctgtggtgggcacacatggaccacctgttgttcagttgcctacaccgtagggacttgatggtcttgctggtgtcgttgaaagactggtgccagtaagttttgttactgatgtcgggccccctccgttcgtgtgtctggtagttagaactgctggccagcgtgaccccgccgctggacttttggtaggacgtatggttgtacggtttacacttgctcaagtacaaatggttattcaaattccgcgcccactaccacagcgcgaacggatgattcctgttagtccacctcgactttatgctcacccacctcaagtgcgacgggctcccgttgatgaggctctggtactggtatctggaatacttgttgcgctagcacctcgtgatgaactttcacccgtctgtcttgccccaagacctttcgctgtagccccatttcaaactgtgggcgttgaagtctgaccccaaactggggcagtgaccagaacagtacggaccccatatatgtttgcttcggaaggtaggtctgtagtaaaacgacggtcctacgccccacctgaagtgggtgtcggcggactcgttgaacaacccgtaggcgttcgccgttgggaaggtcctcccgaaatcctagtggatgctactagacctcccaccattgtaagggcgtgacaacctacacctgcggatggtccgctcgaactttctactgtggcttgtcccgcccccaccgcgtccgccgtcgttgtcgtcaccgtcgccgcgccttctcttgaggttgcgccgtcggcgccgttacgtcggccacctcctgtacttgctagtacggtaagcgccgctgtggaaacggtgtgcccgactcctcttcgcgcgactccggcttcgtcgccggcttcgacggcgggggcgacgcgttgggctccagctcttcggagtcttctttggccactagtttggggactgtctcctgtcgttctttgcgtcaatgttggattattcgttactgtcgtggaagtgggtcatggcgtcgaccatggaacgtatgttgatgccgctgggagtctggccttaggcgagtacctgggacgaaacgtgaggactgcattggacgccgagcctcgtccagatgaccagcaacggtctgtactacgttctggggcactggaaggcgaggtgcgcggtctagtcgttgaaaggccaccacccgcggctcgacaacgggcacgtgaggttctcgaagatgttgctggtccggcagatgagggttgagtaggcggtcaaatggagagactgggtgcacaagttagcgaaagggctcttggtctaaaaccgcgcgggcggtcgggggtggtagtggtggcagtcacttttgcaaggacgagagtgtctagtgccctgcgatggcgacgcgttgtcgtagcctcctcaggtcgctcactggtaatgactgcggtctgcggcgtggacggggatgcaaatgttccgggacccgtatcagagcggcgcgcaggatagctcggcgtgaaaaactcgttcgtacaggtaggaatatagcgggtcgttattgtgtccgaccccggacgcgaagggttcgttctacaaaccgccccggttcttcgcgaggctggttgtgggtcacgcgcacgcgcccgtgatggcgcgcgggaccccgcgcgtgtttgcgccggcgtgacccgcgtggtggcagctactgcggtagctgcgccaccacctcctccgcgcgttgatgtgcgggtgcggcggtggtcacaggtgtcacctgcgccggtaagtctggcaccacgcgcctcgggccgcgatacgattttacttctctgccgcctccgcgcatcgtgcagcggtggcggcggctgggccgtgacggcgggttgcgcgccgccgccgggacgaattggcgcgtgcagcgtggccggctgcccgccggtacgcccggcgagcttccgaccggcgcccataacagtgacacggggggtccaggtccgctgctcgccggcggcgtcgtcggcgccggtaatcacgatactgagtcccagcgtccccgttgcacataacccacgcgctgagccaatcgccggacgcgcacgggcacgcgtgggcggggggcgcgttgatctaacgttcttttttgatgaatctgagcatgacaacatacataggtcgccgccgccgcgcgttgcttcgatacaggttcgcgttttagtttcttctctacgaggtccagtagcgcggcctctagataccggggggcttcttccttctcgtcctaatgttcggggctttcgatttcgcccagtttttctttttctttctactactactacttgaactgctgctccaccttgacgacgtgcgatggcgcgggtccgctgcccatgtcacctttccagctgcgcattttgcacaaaacgctgggccgtggtggcatcagaaatgcgggccactcgcgaggtgggcgtggatgttcgcgcacatactactccacatgccgctgctcctggacgaactcgtccggttgctcgcggagcccctcaaacggatgcctttcgccgtattcctgtacgaccgcaacggcgacctgctcccgttgggttgtggatcggatttcgggcattgtgacgtcgtccacgacgggcgcgaacgtggcaggcttcttttcgcgccggatttcgcgctcagaccactgaaccgtgggtggcacgtcgactaccatgggttcgcggtcgctgaccttctacagaaccttttttactggcaccttggacccgacctcgggctccaggcgcacgccggttagttcgtccaccgcggccctgacccgcacgtctggcacctgcaagtctatgggtgatggtcatcgtggtcataacggtggcggtgtctcccgtacctctgtgtttgcaggggccaacggagtcgccaccgcctacggcgccacgtccgccagcgacgccggcgcaggttctggagatgcctccacgtttgcctgggcacctacaaagcgcaaagtcggggggccgcgggcgcggcaagctccttcatgccgcggcggtcgcgcgatgacgggcttatacgggatgtaggaaggtaacgcggatgggggccgatagcaccgatgtggatggcggggtcttctgctcgttgatgggctgcggcttggtggtgaccttgggcggcggcggcagcggcagcggtcgggcacgaccggggctaaaggcacgcgtcccaccgagcgcttcctccgtcctgggaccacgacggttgtcgcgcgatggtggggtcgtagcaaattttcggccagaaacaccaagaacgtctataccgggagtggacggcggaggcaaagggccacggccctaaggctccttcttacgtggcatcctccccgtaccggccggtgccggactgcccgccgtacgcagcacgcgtggtggccgccgccgcgcgcagcgtggcagcgtacgcgccgccataggacggggaggaataaggtgactagcggcgccgctaaccgcggcacgggccttaacgtaggcaccggaacgtccgcgtctctgtgactaatttttgttcaacgtacacctttttagttttatttttcagacctgagagtgcgagcgaaccaggacattgataaaacatcttaccttctgtagttgaaacgcagagaccggggcgctgtgccgagcgcgggcaagtaccctttgaccgttctatagccgtggtcgttatactcgccaccgcggaagtcgaccccgagcgacacctcgccgtaatttttaaagccaaggtggcaattcttgataccgtcgttccggaccttgtcgtcgtgtccggtctacgactccctattcaactttctcgttttaaaggttgttttccaccatctaccggaccggagaccgtaatcgccccaccacctggaccggttggtccgtcacgttttattctaattgtcattcgaactaggggcgggagggcatctcctcggaggtggccggcacctctgtcacagaggtctccccgcaccgcttttcgcaggcgcggggctgtcccttctttgagaccactgcgtttatctgctcggagggagcatgctcctccgtgatttcgttccggacgggtggtgggcagggtagcgcgggtaccgatggcctcacgacccggtcgtgtgtgggcattgcgacctggacggaggggggcggctgtgggtcgtctttggacacgacggtccgggctggcggcaacaacattgggcaggatcggcgcgcagggacgcggcgcggcggtcgccaggcgctagcaacgccgggcatcggtcaccgttgaccgtttcgtgtgacttgtcgtagcacccagacccccacgttagggacttcgcggctgctacgaagactatcgattgcacagcatacacacagtacatacgcaggtacagcggcggtctcctcgacgactcggcggcgcgcgggcgaaaggttctaccgatggggaagctactacggcgtcaccagaatgtacgtgtagagcccggtcctgcggagcctcatggactcggggcccgaccacgtcaaacgggcgcggtggctctgcatgaagtcggacttattgttcaaatctttggggtgccaccgcggatgcgtgctgcactggtgtctggccagggtcgcaaactgcgacgccaagtagggacacctggcactcctatgacgcatgagcatgttccgcgccaagtgggatcgacacccactattggcacacgacctgtaccgaaggtgcatgaaactgtaggcgccgcacgacctgtccccgggatgaaaattcgggatgagaccgtgacggatgttgcgggaccgagggttcccacggggtttaggaacgcttaccctacttcgacgatgacgagaactttatttggatcttcttctcctgctactgttgcttctgcttcatctgctcgttcgactcgtcgttttttgagtgcataaacccgtccgcggaataagaccatatttataatgtttcctcccataagtttatccacagcttccagtttgtggatttatacggctattttgtaaagttggacttggagtttatcctcttagagtcaccatgctttgtctttaattagtacgtcgaccctctcaggattttttctgatggggttactttggtacaatgccaagtatacgttttgggtgtttacttttacctcccgttccgtaagaacatttcgttgttttacctttcgatctttcagttcacctttacgttaaaaagagttgatgactccgtcggcgtccgttaccactattgaactgaggatttcaccataacatgtcacttctacatctatatctttggggtctgtgagtataaagaatgtacgggtgataattccttccattgagtgctcttgattacccggttgttagatacgggttgtccggattaatgtaacgaaaatccctgttaaaataaccagattacataatgttgtcgtgcccattatacccacaagaccgcccggttcgtagcgtcaacttacgacaacatctaaacgttctgtctttgtgtctcgaaagtatggtcgaaaacgaactaaggtaaccactatcttggtccatgaaaagatacaccttagtccgacaactgtcgatactaggtctacaatcttaataacttttagtaccttgacttctacttgaaggtttaatgacgaaaggtgaccctccacactaattatgtctctgagaatggttccattttggattttgtccagtccttttacctaccctttttctacgatgtcttaaaagtctatttttactttattctcaacctttattaaaacggtacctttagttagatttacggttggacacctctttaaaggacatgaggttgtatcgcgacataaacgggctgttcgatttcatgtcaggaaggttgcatttttaaagactattgggtttgtggatgctgatgtacttgttcgctcaccaccgagggcccgatcacctgacgatgtaattggaacctcgtgcgaccagggaactgatatacctgttgcagttgggtaaattggtggtggcgttacgaccggacgcgatggcgagttacaacgacccgttaccagcgatacacgggaaggtgtaggtccacggagtcttcaagaaacggtaatttttggaggaagaggacggcccgagtatgtggatgctcaccttgaagtccttcctacaattgtaccaagacgtctcgagggatcctttactggattcccaactgcctcggtcgtaattcaaactatcgtaaacggaaatgcggtggaagaaggggtaccgggtgttgtggcggaggtgcgaactccggtacgaatctttgctgtggttgctggtcaggaaattgctgatagagaggcggcggttgtacgagatgggatatgggcggttgcgatggttgcacgggtataggtaggggagggcgttgacccgccgaaaggcgccgacccggaagtgcgcggaattctgattcctttggggtagtgacccgagcccgatgctgggaataatgtggatgagaccgagatatgggatggatctaccttggaaaatggagttggtgtggaaattcttccaccggtaatggaaactgagaagacagtcgaccggaccgttactggcggacgaatgggggttgctcaaactttaattcgcgagtcaactgcccctcccaatgttgcaacgggtcacattgtactggtttctgaccaaggaccatgtttacgatcgattgatattgtaaccgatggtcccgaagatatagggtctctcgatgttcctggcgtacatgaggaagaaatctttgaaggtcgggtactcggcagtccaccacctactatgatttatgttcctgatggttgtccacccgtaggatgtggttgtgttgttgagacctaaacaaccgatggaacgggggtggtacgcgcttcctgtccggatgggacgattgaaggggataggcgaatatccgttctggcgtcaactgtcgtaatgggtctttttcaaagaaacgctagcgtgggaaaccgcgtagggtaagaggtcattgaaatacaggtacccgcgtgagtgtctggacccggttttggaagagatgcggttgaggcgggtgcgcgatctgtactgaaaactccacctagggtacctgctcgggtgggaagaaatacaaaacaaacttcagaaactgcaccaggcacacgtggtcggcgtggcgccgcagtagctttggcacatggacgcgtgcgggaagagccggccgttgcggtgttgtatttcttcgttcgttgtagttgttgtcgacggcggtacccgaggtcactcgtccttgactttcggtaacagtttctagaaccaacacccggtataaaaaacccgtggatactgttcgcgaaaggtccgaaacaaagaggtgtgttcgagcggacgcggtatcagttatgccggccagcgctctgacccccgcatgtgacctaccggaaacggaccttgggcgtgagtttttgtacgatggagaaactcgggaaaccgaaaagactggtcgctgagttcgtccaaatggtcaaactcatgctcagtgaggacgcggcatcgcggtaacgaagaagggggctggcgacatattgcgaccttttcaggtgggtttcgcatgtccccgggttgagccggcggacacctgataagacgacgtacaaagaggtgcggaaacggttgaccggggtttgagggtacctagtgttggggtggtacttggaataatggccccatgggttgaggtacgagttgtcaggggtccatgtcgggtgggacgcagcgttggtccttgtcgagatgtcgaaggacctcgcggtgagcgggatgaaggcgtcggtgtcacgcgtctaatcctcgcggtgaagaaaaacagtgaactttttgtacatttttattacatgatctctgtgaaagttatttccgtttacgaaaataaacatgtgagagcccactaataaatgggggtgggaacggcagacgcggcaaatttttagtttccccaagacggcgcgtagcgatacgcggtgaccgtccctgtgcaacgctatgaccacaaatcacgaggtgaatttgagtccgtgttggtaggcgccgtcgagccacttcaaaagtgaggtgtccgacgcgtggtagtggttgcgcaaatcgtccagcccgcggctatagaacttcagcgtcaaccccggaggcgggacgcgcgcgctcaacgctatgtgtcccaacgtcgtgaccttgtgatagtcgcggcccaccacgtgcgaccggtcgtgcgagaacagcctctagtctaggcgcaggtccaggaggcgcaacgagtcccgcttgcctcagttgaaaccatcgacggaagggtttttcccgcgcacgggtccgaaactcaacgtgagcgtggcatcaccgtagttttccactggcacgggccagacccgcaatcctatgtcgcggacgtattttcggaactagacgaattttcggtggactcggaaacgcggaagtctcttcttgtacggcgttctgaacggccttttgactaaccggcctgtccggcgcagcacgtgcgtcgtggaacgcagccacaacctctagacgtggtgtaaagccggggtggccaagaagtgctagaaccggaacgatctgacgaggaagtcgcgcgcgacgggcaaaagcgagcagtgtaggtaaagttagtgcacgaggaataaatagtattacgaaggcacatctgtgaattcgagcggaagctagagtcgcgtcgccacgtcggtgttgcgcgtcgggcacccgagcactacgaacatccagtggagacgtttgctgacgtccatgcggacgtccttagcggggtagtagcagtgtttccagaacaacgaccacttccagtcgacgttgggcgccacgaggagcaagtcggtccagaacgtatgccggcggtctcgaaggtgaaccagtccgtcatcaaacttcaagcggaaatctagcaataggtgcaccatgaacaggtagtcgcgcgcgcgtcggaggtacgggaagagggtgcgtctgtgctagccgtgtgagtcgcccaagtagtggcattaaagtgaaaggcgaagcgacccgagaaggagaaggagaacgcaggcgtatggtgcgcggtgacccagcagaagtaagtcggcggcgtgacacgcgaatggaggaaacggtacgaactaatcgtggccacccaacgactttgggtggtaaacatcgcggtgtagaagagaaagaaggagcgacaggtgctaatggagaccactaccgcccgcgagcccgaaccctcttcccgcgaagaaaaagaagaacccgcgttaccggtttaggcggcggctccagctaccggcgcccgacccacacgcgccgtggtcgcgcagaacactactcagaaggagcaggagcctgagctatgcggcggagtaggcgaaaaaacccccgcgggcccctccgccgccgctgcccctgcccctgctgtgcaggaggtaccaaccccctgcagcgcggcgtggcgcaggcgcgagcccccaccaaagcgcgacgaggagaagggctgaccggtaaaggaagaggatatccgtctttttctagtacctcagtcagctcttcttcctgtcggattggcgggggagactcaagcggtggtggcggaggtggctacggcggttgcgcggatggtggaaggggcagctccgtgggggcgaactcctcctccttcactaatagctcgtcctgggtccaaaacattcgcttctgctgctcctggcgagtcatggttgtctcctatttttcgttctggtcctgttgcgtctccgtttgctccttgttcagcccgcccccctgctttccgtaccgctgatggatctacaccctctgctgcacgacaacttcgtagacgtcgcggtcacgcggtaatagacgctgcgcaacgttctcgcgtcgctacacggggagcggtatcgcctacagtcggaacggatgcttgcggtggataagagtggcgcgcatggggggtttgcggttcttttgccgtgtacgctcgggttgggcgcggagttgaagatggggcataaacggcacggtctccacgaacggtggatagtgtagaaaaaggttttgacgttctatggggataggacggcacggttggcgtcggctcgcctgttcgtcgaccggaacgccgtcccgcgacagtatggactatagcggagcgagttgcttcacggtttttagaaactcccagaacctgcgctgctcttcgcgcgccgtttgcgagacgttgtccttttgtcgcttttactttcagtgagacctcacaaccaccttgagctcccactgttgcgcgcggatcggcatgattttgcgtcgtagctccagtgggtgaaacggatgggccgtgaattggatggggggttccagtactcgtgtcagtactcactcgactagcacgcggcacgcgtcggggacctctccctacgtttaaacgttcttgtttgtctcctcccggatgggcgtcaaccgctgctcgtcgatcgcgcgaccgaagtttgcgcgctcggacggctgaacctcctcgctgcgtttgattactaccggcgtcacgagcaatggcacctcgaactcacgtacgtcgccaagaaacgactgggcctctacgtcgcgttcgatctcctttgtaacgtgatgtggaaagctgtcccgatgcatgcggtccggacgttctagaggttgcacctcgagacgttggaccagaggatggaaccttaaaacgtgcttttggcggaacccgttttgcacgaagtaaggtgcgagttcccgctccgcgcggcgctgatgcaggcgctgacgcaaatgaataaagatacgatgtggaccgtctgccggtacccgcaaaccgtcgtcacgaacctcctcacgttggagttcctcgacgtctttgacgatttcgttttgaacttcctggatacctgccggaagttgctcgcgaggcaccggcgcgtggaccgcctgtagtaaaaggggcttgcggacgaattttgggacgttgtcccagacggtctgaagtggtcagtttcgtacaacgtcttgaaatccttgaaataggatctcgcgagtccttagaacgggcggtggacgacacgtgaaggatcgctgaaacacgggtaattcatggcgcttacgggaggcggcgaaaccccggtgacgatggaagacgtcgatcggttgatggaacggatggtgagactgtattaccttctgcactcgccactgccagatgacctcacagtgacagcgacgttggatacgtggggcgtggcgagggaccaaacgttaagcgtcgacgaattgctttcagtttaatagccatggaaactcgacgtcccagggagcggactgcttttcaggcgccgaggccccaactttgagtgaggccccgacacctgcagccgaatggaagcgtttaaacatggactcctgatggtgcgggtgctctaatccaagatgcttctggttagggcgggcggattacgcctcgaatggcggacgcagtaatgggtcccggtgtaagaaccggttaacgttcggtagttgtttcgggcggttctcaaagacgatgctttccctgccccccaaatgaacctgggggtcaggccgctcctcgagttgggttaggggggcggcggcgtcgggatagtcgtcgtcggcgcccgggaacgaagggtcctaccgtgggtttttcttcgacgtcgacggcggcggtgggtgcctgctcctccttatgaccctgtcagtccgtctcctccaaaacctgctcctcctcctcctgtactaccttctgaccctctcggatctgctccttcgaaggctccagcttctccacagtctgctttgtggcagtgggagccagcgtaaggggagcggccgcggggtctttagccgttggccaaggtcgtaccgatgttggaggcgaggagtccgcggcggccgtgacgggcaagcggctgggttggcatctaccctgtggtgaccttggtcccggccattcaggttcgtcggcggcggcaatcgggttctcgttgttgtcgcggttccgatggcgagtaccgcgcccgtgttcttgcggtatcaacgaacgaacgttctgacacccccgttgtagaggaagcgggcggcgaaagaagagatggtagtgccgcaccggaagggggcattgtaggacgtaatgatggcagtagagatgtcgggtatgacgtggccgccgtcgccgtcgttgtcgtcgccggtgtgtcttcgtttccgctggcctatcgttctgagactgtttcgggttctttaggtgtcgccgccgtcgtcgtcctcctcctcgcgacgcagaccgcgggttgcttgggcatagctgggcgctcgaatctttgtcctaaaaagggtgagacatacgatataaagttgtctcgtccccggttcttgttctcgacttttattttttgtccagagacgctagggagtgggcgtcgacggacatagtgttttcgcttctagtcgaagccgcgtgcgaccttctgcgcctccgagagaagtcatttatgacgcgcgactgagaattcctgatcaaagcgcgggaaagagtttaaattcgcgcttttgatgcagtagaggtcgccggtgtgggccgcggtcgtggacaacagtcgcggtaatactcgttcctttaagggtgcgggatgtacacctcaatggtcggtgtttaccctgaacgccgacctcgacgggttctgatgagttgggcttatttgatgtactcgcgccctggggtgtactatagggcccagttgccttatgcgcgggtggctttggcttaagaggaccttgtccgccgataatggtggtgtggagcattattggaattaggggcatcaaccgggcgacgggaccacatggtcctttcagggcgagggtggtgacaccatgaagggtctctgcgggtccggcttcaagtctactgattgagtccccgcgtcgaacgcccgccgaaagcagtgtcccacgccagcgggcccgtcccatattgagtggactgttagtctcccgctccataagtcgagttgctgctcagccactcgaggagcgaaccagaggcaggcctgccctgtaaagtctagccgccgcggccggcgagaagtaagtgcggagcagtccgttaggattgagacgtctggagcaggagactcggcgcgagacctccgtaaccttgagacgttaaataactcctcaaacacggtagccagatgaaattggggaagagccctggagggccggtgataggcctagttaaataaggattgaaactgcgccatttcctgagccgcctgccgatgctgacttacaattcacctctccgtctcgttgacgcggactttgtggaccaggtgacagcggcggtgttcacgaaacgggcgctgaggccactcaaaacgatgaaacttaacgggctcctagtatagctcccgggccgcgtgccgcaggccgaatggcgggtccctctcgaacgggcatcggactaagccctcaaatgggtcgcgggggacgatcaactcgccctgtcccctgggacacaagagtgacactaaacgttgacaggattgggacctaatgtagttctagaaacaacggtagagacacgactcatattatttatgtctttaattttatatgaccccgaggatagcggtaggacatttgcggtggcagaagtgggcgggttcgtttggttccgcttggaatggaccatgaaaattgtagagagggagacactaaatgttgtcaaagttgggtctgcctcactcagatgctctcttggagaggctcgagtcgatgaggtagtcttttttgtggtgggaggaatggacggcccttgcatgctcacgcagtggccggcgacgtggtgtggatggcggactggcatttggtctgaaaaaggcctgtctggagttattgagacaaatggtcttgtcctccactcgaatcttttgggaatcccataatccggtttccgcgtcgatgacaccccaaatacttgttaagttcgttgagatgcccgataagattaagtccaaagagatcttagccccaaccccaataagagacagaacactaagagaaataagaatatgattgcgaagagacggattccgagcggcggacgacacacgtgtaaacgtaaataacagtcgaaaaatttgcgaccccagcggtgggttctactaatccatgtattaggatccaaatgagtgggaacgcagtcgggtgccatggtgggttttccacctaaaattcctcggtcggacattacaatgtaagcgtcgacttcgattactcacgtggtgagaatattttacgtggtgtcttgtacttttcgacgaataagcggtgtttttgttttaaccgttcatacgacaaatacgataaaccgtcggtccactgtgatgtctcatattacaatgtcaaaaggtcccattttcagtattttgaaaatacatatgaaaaggtaaaatactttacacgctgtaatggtacatgtactcgtttgtcatattcaacaccgggggtgttttaacacaccttttgtgaccgtgaaagacgacgtgacgatacgattaatgtcacgagcgaaaccagacatgggatgagatataatttatgttttcgtctgcgtcgaaataactccttttcttttacggaattaaatgattcaatgtttcgattacagtggtgattgacgaaatgagcgacgaacgttttgtttaagtttttcaatcgtaatattaatcttatcctaaatttggggggccagtaaaggacgagttatggtaaggggacttgttaactgagatacaccctatacgaggtcgcgatgttggaacttcagtccgaaggacctacagtcgtagactgaaaccggtcgtggacagggcgcctaaacaaggtcaggttgatgtcgctgggtgggattgtctctactggttgtgttggttgcgccggcggcgatggcctgaatgtagatggtgtttatgtggggttcaaagacggaaacagttattgaccctattgaacccgtacaccaccaagaggtatcgcgaatacaaacatacggaataataatacaccgagtagacgacggatttcgcgtttgcgcgggctggtgggtagatatcagggtagtaacacgatgtgggtttgttactaccttaggtatctaacctgcctgactttgtgtacaagaaaagagaatgtcatactaatttactctgtactaaggagctcaaaaatataatgactgggaacaacgcgaaaaaacacgcacgaggtgtaaccgacgccaaagagtgtagcttcatctgacgtaaggtcggaagtgtcagataaacgaaatgcctaaacagtgggagtgcgagtagacgtcggagtagtgacaccagtagcggaaataggtcacgtaactgacccagacacacgcgaaacgtatagagtctgtggtaggggtcatgtccctgtcctgatatcgactcgaagaatcttaagaaattaatactttaaatgacactgaaaagacgactaataaacgtgggatagacgcaaaacaaggggctggaggttcggagtttctgtatatagtacgtctaagtgagcatataccttataaggttcaacgatgttacttttttcgctagaaaggcttcggaccaatatacgttagtagagacaataccacaagacgtcatggtagaatcgggatcgatatatagggatggaactgtaaccgaccttgcgttatctacggtacttggtgggttgaaaggggcgcgggcgatacgaaggtgacgttgttcaacaacggccgccgaaacagggtcggttagtcggagcgggtggaagagggtgggggtgactttagtcgatgaaattagattgtcctcctctactgactgtgggatctagatctttacctgccttaataatgtctcgtcgcggacgatctttctgcgtcccgtcgccggctcgttgtcgcgtacttagttctcgaggttctgtaccaattgaacgtggtcacgttttccccatagaaaacagagcatttcgtccggtttcagtggatgctgtcattatggtggcctgtggcggaatcgatgttcaacggttggttcgcagtctttaaccaccagtaccaccctcttttcgggtaatggtattgagtcgtgagccatctttggcttccgacgtaagtgagtggaacagttcctggactcctagagacgtgggaataattctgggacacgccagagtttctagaataagggaaattgattatttttttttattatttcgtagtgaatgaattttagtcaatcgtttaaagacaggtcaaataagtcgtcgtggaggaacgggaggagggtcgagaccataacgtcgaaggaggaccgacgtttgaaagaggtgttagatttaccttacagtcaaaggaggacaaggacaggtaggcgtgggtgatagaagtacaacaacgtctacttcgcgcgttctggcagacttctatggaagttggggcacataggtatactgtgcctttggccaggaggttgacacggaaaagaatgaggagggaaacatagggggttacccaaagttctctcagggggaccccatgagagaaacgcggataggcttggagatcaatggaggttaccgtacgaacgcgagttttacccgttgccggagagagacctgctccggccgttggaatggagggttttacattggtgacactcgggtggagagtttttttggttcagtttgtatttggacctttatagacgtggggagtgtcaatggagtcttcgggattgacaccgacggcggcgtggagattaccagcgcccgttgtgtgagtggtacgttagtgtccggggcgattggcacgtgctgaggtttgaatcgtaacggtgggttcctggggagtgtcacagtcttcctttcgatcgggacgtttgtagtccgggggagtggtggtggctatcgtcatgggaatgatagtgacggagtgggggaaattgatgacggtgaccatcgaacccgtaactgaactttctcgggtaaatatgtgttttaccttttgatcctgatttcatgccccgaggaaacgtacattgtctgctggatttgtgaaactggcatcgttgaccaggtccacactgataattattatgaaggaacgtttgatttcaatgacctcggaacccaaaactaagtgttccgttatacgttgaattacatcgtcctcctgattcctaactaagagttttgtctgcggaatatgaactacaatcaataggcaaactacgagttttggttgatttagattctgatcctgtcccgggagaaaaatatttgagtcgggtgttgaacctataattgatgttgtttccggaaatgaacaaatgtcgaagtttgttaaggtttttcgaactccaattggattcgtgacggttccccaactacaaactgcgatgtcggtatcggtaattacgtcctctacccgaacttaaaccaagtggattacgtggtttgtgtttaggggagttttgtttttaaccggtaccggatcttaaactaagtttgttccgataccaaggatttgatccttgaccggaatcaaaactgtcgtgtccacggtaatgtcatcctttgtttttattactattcgattgaaacacctggtgtggtcgaggtagaggattgacatctgatttacgtctctttctacgatttgagtgaaaccagaattgttttacaccgtcagtttatgaacgatgtcaaagtcaaaaccgacaatttccgtcaaaccgaggttatagaccttgtcaagtttcacgagtagaataatattctaaactgcttttacctcacgatgatttgttaaggaaggacctgggtcttataaccttgaaatctttacctctagaatgacttccgtgtcggatatgtttgcgacaacctaaatacggattggatagtcgaataggttttagagtgccattttgacggttttcattgtaacagtcagttcaaatgaatttgcctctgttttgatttggacattgtgattggtaatgtgatttgccatgtgtcctttgtcctctgtgttgaggttcacgtatgagatacagtaaaagtaccctgaccagaccggtgttgatgtaattactttataaacggtgtaggagaatgtgaaaaagtatgtaacgggttcttatttcttagcaaacacaatacaaagttgcacaaataaaaagttaacgtcttttaaagttcagtaaaaagtaagtcatcatatcggggtggtggtgtatcgaatatgtctagtggcatggaattagtttgagtgtcttgggatcataagttggacggtggagggagggttgtgtgtctcatgtgtcaggaaagaggggccgaccggaatttttcgtagtatagtacccattgtctgtataagaatccacaatataaggtgtgccaaaggacagctcggtttgcgagtagtcactataattatttgaggggcccgtcgagtgaattcaagtacagcgacaggtcgacgactcggtgtccgacgacaggttgaacgccaacgaattgcccgccgcttcctcttcaggtgcggatgtacccccatctcagtattagcacgtagtcctatcccgccaccacgacgtcgtcgcgcgcttatttgacgacggcggcggcgaggcaggacgtccttatgttgtaccgtcaccagaggagtcgctactaagcgtggcgggcgtcgtattccgcggaacaggaggcccgtgtcgtcgcgtgggactagagtgaatttagtcgtgtcattgacgtcgtgtcgtggtgttataacaagttttagggtgtcacgttccgcgacataggtttcgagtaccgcccctggtgtcttgggtgcaccggtagtatggtgttcgcgtccatctaattcaccgctggggagtatttgtgcgacctgtatttgtaatggagaaaaccgtacaacattaagtggtggagggccatggtatatttggagactaatttgtaccgcggtaggtggtggtaggatttggtcgaccggttttggacgggcggccgatatgtgacgtcccttggccctgaccttgttactgtcacctctcgggtcctgagcattggtacctagtagtacgagcagtactatagttacaaccgtgttgtgtccgtgtgcacgtatgtgaaggagtcctaatgttcgaggagggcgcaatcttggtatagggtcccttgttgggtaaggacttagtcgcatttagggtgtgacgtcccttctggagcgtgcattgagtgcaacacgtaacagtttcacaatgtaagcccgtcgtcgcctactaggaggtcataccatcgcgcccaaagacagagttttcctccatctgctagggatgacatgcctcacgcggctctgttggctctagcacaaccagcatcacagtacggtttaccttgcggcctgcatcagtataaaggacttcgttttggtccacgcccgcactgtttgtctagacgcagaggccagagcggcgaatctagcgagacacatcatcaacatcatataggtgagagagtttcgtaggtccgcgggggaccgaagcccaagatacatttgaggaagtacgcggcgacgggactattgtaggtggtggcgtcttattcggtgtgggtcggttggatgtgtaagcaagacgctcagtgtgtgccctcctcgcccttctcgaccttcttggtacaaaaaaaaaaataaggttttctaataggttttggagttttacttctagataattcacttgcgcgaggggaggccaccgcaccagtttgagatgtcggtttcttgtctattaccgtaaacattctacaacgtgttaccgaaggttttccgtttgccgggagtgcaggttcacctgcatttccgatttgggaagtcccacttagaggagatatttgtaaggtcgtggaagttggtacgggtttattaagagtagagcggtggaagagttatatagagattcgtttagggcttataattcaggccggtaacatttttagacgaggtctcgcgggaggtggaagtcggagttcgtcgcttagtactaacgtttttaagtccaaggagtgtctggacatattctaagttttcgccttgtaattgtttttatggcgctagggcatccagggaagcgtcccggtcgacttgtattagcacgtccagacgtgcctggtcgcgccggtgaaggggcggtccttggtactgttttcttgggtgtgactaatactgtgcgtatgagcctcgatacgattggtcgcatcggggctacattcgaacaacgtacccgccgctatattttacgttccacgacgagttttttagtccgtttcggagcgcgttttttctttcgtgtagcatcagtacgagtacgtctatttccgtccattcgaggccttggtggtgtctttttctgtggtaaaaagagagtttgtacagacgcccaaagacgtatttgtgttttattttattgtttttttgtaaatttgtaatcttcggacagaatgttgtcctttttgttgggaatattcgtattctgcctgatgccggtacggccgcactggcatttttttgaccagtggcactaatttttcgtggtggctgtcgaggagccagtacaggcctcagtattacattctgagccatttgtgtagtccaactaagtgtagccagtcacgatttttcgctggctttatcgggcccccttatgtatgggcgtccgcatctctgttgtaatgtcgggggtatcctccatattgttttaattatcctctctttttgtgtatttgtggactttttgggaggacggatccgttttatcgtgggagggcgaggtcttgttgtatgtcgcgaaggtgtcgccgtcggtattgtcagtcggaatggtcattttttcttttggataatttttttgtggtgagctgtgccgtggtcgagttagtcagtgtcacattttttcccggttcacgtctcgctcatatatatcctgattttttactgcattgccaatttcaggtgttttttgtgggtcttttggcgtgcgcttggatgcgggtctttgctttcggttttttgggtgttgaaggagtttagcagtgaaggcaaaagggtgcaatgcagtgaagggtaaaattcttttgatgttaagggttgtgtatgttcaatgaggcgggattttggatgcagtgggcggggcaagggtgcggggcgcggtgcagtgtttgaggtgggggagtaatagtataaccgaagttaggttttattccatataataactactacaattaattcttaagcctagacgctgcgctccgacctaccggaaggggtaatactaagaagagcgaaggccgccgtagccctacgggcgcaacgtccggtacgacaggtccgtccatctactgctggtagtccctgtcgaagttccggtcgttttccggtccttggcatttttccggcgcaacgaccgcaaaaaggtatccgaggcggggggactgctcgtagtgtttttagctgcgagttcagtctccaccgctttgggctgtcctgatatttctatggtccgcaaagggggaccttcgagggagcacgcgagaggacaaggctgggacggcgaatggcctatggacaggcggaaagagggaagcccttcgcaccgcgaaagagtatcgagtgcgacatccatagagtcaagccacatccagcaagcgaggttcgacccgacacacgtgcttggggggcaagtcgggctggcgacgcggaataggccattgatagcagaactcaggttgggccattctgtgctgaatagcggtgaccgtcgtcggtgaccattgtcctaatcgtctcgctccatacatccgccacgatgtctcaagaacttcaccaccggattgatgccgatgtgatcttcctgtcataaaccatagacgcgagacgacttcggtcaatggaagcctttttctcaaccatcgagaactaggccgtttgtttggtggcgaccatcgccaccaaaaaaacaaacgttcgtcgtctaatgcgcgtctttttttcctagagttcttctaggaaactagaaaagatgccccagactgcgagtcaccttgcttttgagtgcaattccctaaaaccagtactctaatagtttttcctagaagtggatctaggaaaatttagttagatttcatatatactcatttgaaccagactgtcaatggttacgaattagtcactccgtggatagagtcgctagacagataaagcaagtaggtatcaacggactgaggggcagcacatctattgatgctatgccctcccgaatggtagaccggggtcacgacgttactatggcgctctgggtgcgagtggccgaggtctaaatagtcgttatttggtcggtcggccttcccggctcgcgtcttcaccaggacgttgaaataggcggaggtaggtcagataattaacaacggcccttcgatctcattcatcaagcggtcaattatcaaacgcgttgcaacaacggtaacgatgtccgtagcaccacagtgcgagcagcaaaccataccgaagtaagtcgaggccaagggttgctagttccgctcaatgtactagggggtacaacacgttttttcgccaatcgaggaagccaggaggctagcaacagtcttcattcaaccggcgtcacaatagtgagtaccaataccgtcgtgacgtattaagagaatgacagtacggtaggcattctacgaaaagacactgaccactcatgagttggttcagtaagactcttatcacatacgccgctggctcaacgagaacgggccgcagttgtgccctattatggcgcggtgtatcgtcttgaaattttcacgagtagtaaccttttgcaagaagccccgcttttgagagttcctagaatggcgacaactctaggtcaagctacattgggtgagcacgtgggttgactagaagtcgtagaaaatgaaagtggtcgcaaagacccactcgtttttgtccttccgttttacggcgttttttcccttattcccgctgtgcctttacaacttatgagtatgagaaggaaaaagttataataacttcgtaaatagtcccaataacagagtactcgcctatgtataaacttacataaatctttttatttgtttatccccaaggcgcgtgtaaaggggcttttcacggtggactgcagattctttggtaataatagtactgtaattggatatttttatccgcatagtgctccgggaaagcagaagttcttaacctaggcttaagaatta

SEQ ID NO: 27 - DNA, 1521 bp, Artificial Sequence
  Codon optimized DNA encoding human HIV-1 gag
atgggtgctagggcttctgtgctgtctggtggtgagctggacaagtgggagaagatcaggctgaggcctggtggcaagaagaagtacaagctaaagcacattgtgtgggcctccagggagctggagaggtttgctgtgaaccctggcctgctggagacctctgaggggtgcaggcagatcctgggccagctccagccctccctgcaaacaggctctgaggagctgaggtccctgtacaacacagtggctaccctgtactgtgtgcaccagaagattgatgtgaaggacaccaaggaggccctggagaagattgaggaggagcagaacaagtccaagaagaaggcccagcaggctgctgctggcacaggcaactccagccaggtgtcccagaactaccccattgtgcagaacctccagggccagatggtgcaccaggccatctccccccggaccctgaatgcctgggtgaaggtggtggaggagaaggccttctcccctgaggtgatccccatgttctctgccctgtctgagggtgccaccccccaggacctgaacaccatgctgaacacagtggggggccatcaggctgccatgcagatgctgaaggagaccatcaatgaggaggctgctgagtgggacaggctgcatcctgtgcacgctggccccattgcccccggccagatgagggagcccaggggctctgacattgctggcaccacctccaccctccaggagcagattggctggatgaccaacaacccccccatccctgtgggggaaatctacaagaggtggatcatcctgggcctgaacaagattgtgaggatgtactcccccacctccatcctggacatcaggcagggccccaaggagcccttcagggactatgtggacaggttctacaagaccctgagggctgagcaggcctcccaggaggtgaagaactggatgacagagaccctgctggtgcagaatgccaaccctgactgcaagaccatcctgaaggccctgggccctgctgccaccctggaggagatgatgacagcctgccagggggtggggggccctggtcacaaggccagggtgctggctgaggccatgtcccaggtgaccaactccgccaccatcatgatgcagaggggcaacttcaggaaccagaggaagacagtgaagtgcttcaactgtggcaaggtgggccacattgccaagaactgtagggcccccaggaagaagggctgctggaagtgtggcaaggagggccaccagatgaaggactgcaatgagaggcaggccaacttcctgggcaaaatctggccctcccacaagggcaggcctggcaacttcctccagtccaggcctgagcccacagcccctcccgaggagtccttcaggtttggggaggagaagaccacccccagccagaagcaggagcccattgacaaggagctgtaccccctggcctccctgaggtccctgtttggcaacgacccctcctcccagtaaaataaagcccgggcagat

SEQ ID NO: 28 - DNA, 38519 bp, Artificial Sequence
  Codon optimized DNA encoding pMRKAd5HIV-1 pol, coding
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattataggcggccgcgatccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgagatctaccatggcccccatctcccccattgagactgtgcctgtgaagctgaagcctggcatggatggccccaaggtgaagcagtggcccctgactgaggagaagatcaaggccctggtggaaatctgcactgagatggagaaggagggcaaaatctccaagattggccccgagaacccctacaacacccctgtgtttgccatcaagaagaaggactccaccaagtggaggaagctggtggacttcagggagctgaacaagaggacccaggacttctgggaggtgcagctgggcatcccccaccccgctggcctgaagaagaagaagtctgtgactgtgctggctgtgggggatgcctacttctctgtgcccctggatgaggacttcaggaagtacactgccttcaccatcccctccatcaacaatgagacccctggcatcaggtaccagtacaatgtgctgccccagggctggaagggctcccctgccatcttccagtcctccatgaccaagatcctggagcccttcaggaagcagaaccctgacattgtgatctaccagtacatggctgccctgtatgtgggctctgacctggagattgggcagcacaggaccaagattgaggagctgaggcagcacctgctgaggtggggcctgaccacccctgacaagaagcaccagaaggagccccccttcctgtggatgggctatgagctgcaccccgacaagtggactgtgcagcccattgtgctgcctgagaaggactcctggactgtgaatgacatccagaagctggtgggcaagctgaactgggcctcccaaatctaccctggcatcaaggtgaggcagctgtgcaagctgctgaggggcaccaaggccctgactgaggtgatccccctgactgaggaggctgagctggagctggctgagaacagggagatcctgaaggagcctgtgcatggggtgtactatgacccctccaaggacctgattgctgagatccagaagcagggccagggccagtggacctaccaaatctaccaggagcccttcaagaacctgaagactggcaagtatgccaggatgaggggggcccacaccaatgatgtgaagcagctgactgaggctgtgcagaagatcaccactgagtccattgtgatctggggcaagacccccaagttcaagctgcccatccagaaggagacctgggagacctggtggactgagtactggcaggccacctggatccctgagtgggagtttgtgaacaccccccccctggtgaagctgtggtaccagctggagaaggagcccattgtgggggctgagaccttctatgtggctggggctgccaacagggagaccaagctgggcaaggctggctatgtgaccaacaggggcaggcagaaggtggtgaccctgactgacaccaccaaccagaagactgccctccaggccatctacctggccctccaggactctggcctggaggtgaacattgtgactgcctcccagtatgccctgggcatcatccaggcccagcctgatcagtctgagtctgagctggtgaaccagatcattgagcagctgatcaagaaggagaaggtgtacctggcctgggtgcctgcccacaagggcattgggggcaatgagcaggtggacaagctggtgtctgctggcatcaggaaggtgctgttcctggatggcattgacaaggcccaggatgagcatgagaagtaccactccaactggagggctatggcctctgacttcaacctgccccctgtggtggctaaggagattgtggcctcctgtgacaagtgccagctgaagggggaggccatgcatgggcaggtggactgctcccctggcatctggcagctggcctgcacccacctggagggcaaggtgatcctggtggctgtgcatgtggcctccggctacattgaggctgaggtgatccctgctgagacaggccaggagactgcctacttcctgctgaagctggctggcaggtggcctgtgaagaccatccacactgccaatggctccaacttcactggggccacagtgagggctgcctgctggtgggctggcatcaagcaggagtttggcatcccctacaacccccagtcccagggggtggtggcctccatgaacaaggagctgaagaagatcattgggcaggtgagggaccaggctgagcacctgaagacagctgtgcagatggctgtgttcatccacaacttcaagaggaaggggggcatcgggggctactccgctggggagaggattgtggacatcattgccacagacatccagaccaaggagctccagaagcagatcaccaagatccagaacttcagggtgtactacagggactccaggaaccccctgtggaagggccctgccaagctgctgtggaagggggagggggctgtggtgatccaggacaactctgacatcaaggtggtgcccaggaggaaggccaagatcatcagggactatggcaagcagatggctggggatgactgtgtggcctccaggcaggatgaggactaaagcccgggcagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccgatcggcgcgccgtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaaacagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaacgcaatagatgccatgaaccacccaactttccccgcgcccgctatgcttccactgcaacaagttgttgccggcggctttgtcccagccaatcagcctcgcccaccttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctcgtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaagaattcggatctgcgacgcgaggctggatggccttccccattatgattcttctcgcttccggcggcatcgggatgcccgcgttgcaggccatgctgtccaggcaggtagatgacgaccatcagggacagcttcaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggatccgaattcttaatttcttaattaa

SEQ ID NO: 29 - DNA, 38519 bp, Artificial Sequence
  Codon optimized DNA encoding pMRKAd5HIV-1 pol, noncoding
gtagtagttattatatggaataaaacctaacttcggttatactattactcccccacctcaaacactgcaccgcgccccgcacccttgccccgcccactgcatcatcacaccgccttcacactacaacgttcacaccgccttgtgtacattcgctgcctacaccgttttcactgcaaaaaccacacgcggccacatgtgtccttcactgttaaaagcgcgccaaaatccgcctacaacatcatttaaacccgcattggctcattctaaaccggtaaaagcgcccttttgacttattctccttcactttagacttattaaaacacaatgagtatcgcgcattataaacagatcccggcgcccctgaaactggcaaatgcacctctgagcgggtccacaaaaagagtccacaaaaggcgcaaggcccagtttcaaccgcaaaataataatatccgccggcgctaggtaacgtatgcaacataggtatagtattatacatgtaaatataaccgagtacaggttgtaatggcggtacaactgtaactaataactgatcaataattatcattagttaatgccccagtaatcaagtatcgggtatatacctcaaggcgcaatgtattgaatgccatttaccgggcggaccgactggcgggttgctgggggcgggtaactgcagttattactgcatacaagggtatcattgcggttatccctgaaaggtaactgcagttacccacctcataaatgccatttgacgggtgaaccgtcatgtagttcacatagtatacggttcatgcgggggataactgcagttactgccatttaccgggcggaccgtaatacgggtcatgtactggaataccctgaaaggatgaaccgtcatgtagatgcataatcagtagcgataatggtaccactacgccaaaaccgtcatgtagttacccgcacctatcgccaaactgagtgcccctaaaggttcagaggtggggtaactgcagttaccctcaaacaaaaccgtggttttagttgccctgaaaggttttacagcattgttgaggcggggtaactgcgtttacccgccatccgcacatgccaccctccagatatattcgtctcgagcaaatcacttggcagtctagcggacctctgcggtaggtgcgacaaaactggaggtatcttctgtggccctggctaggtcggaggcgccggcccttgccacgtaaccttgcgcctaaggggcacggttctcactctagatggtaccgggggtagagggggtaactctgacacggacacttcgacttcggaccgtacctaccggggttccacttcgtcaccggggactgactcctcttctagttccgggaccacctttagacgtgactctacctcttcctcccgttttagaggttctaaccggggctcttggggatgttgtggggacacaaacggtagttcttcttcctgaggtggttcacctccttcgaccacctgaagtccctcgacttgttctcctgggtcctgaagaccctccacgtcgacccgtagggggtggggcgaccggacttcttcttcttcagacactgacacgaccgacaccccctacggatgaagagacacggggacctactcctgaagtccttcatgtgacggaagtggtaggggaggtagttgttactctggggaccgtagtccatggtcatgttacacgacggggtcccgaccttcccgaggggacggtagaaggtcaggaggtactggttctaggacctcgggaagtccttcgtcttgggactgtaacactagatggtcatgtaccgacgggacatacacccgagactggacctctaacccgtcgtgtcctggttctaactcctcgactccgtcgtggacgactccaccccggactggtggggactgttcttcgtggtcttcctcggggggaaggacacctacccgatactcgacgtggggctgttcacctgacacgtcgggtaacacgacggactcttcctgaggacctgacacttactgtaggtcttcgaccacccgttcgacttgacccggagggtttagatgggaccgtagttccactccgtcgacacgttcgacgactccccgtggttccgggactgactccactagggggactgactcctccgactcgacctcgaccgactcttgtccctctaggacttcctcggacacgtaccccacatgatactggggaggttcctggactaacgactctaggtcttcgtcccggtcccggtcacctggatggtttagatggtcctcgggaagttcttggacttctgaccgttcatacggtcctactccccccgggtgtggttactacacttcgtcgactgactccgacacgtcttctagtggtgactcaggtaacactagaccccgttctgggggttcaagttcgacgggtaggtcttcctctggaccctctggaccacctgactcatgaccgtccggtggacctagggactcaccctcaaacacttgtgggggggggaccacttcgacaccatggtcgacctcttcctcgggtaacacccccgactctggaagatacaccgaccccgacggttgtccctctggttcgacccgttccgaccgatacactggttgtccccgtccgtcttccaccactgggactgactgtggtggttggtcttctgacgggaggtccggtagatggaccgggaggtcctgagaccggacctccacttgtaacactgacggagggtcatacgggacccgtagtaggtccgggtcggactagtcagactcagactcgaccacttggtctagtaactcgtcgactagttcttcctcttccacatggaccggacccacggacgggtgttcccgtaacccccgttactcgtccacctgttcgaccacagacgaccgtagtccttccacgacaaggacctaccgtaactgttccgggtcctactcgtactcttcatggtgaggttgacctcccgataccggagactgaagttggacgggggacaccaccgattcctctaacaccggaggacactgttcacggtcgacttccccctccggtacgtacccgtccacctgacgaggggaccgtagaccgtcgaccggacgtgggtggacctcccgttccactaggaccaccgacacgtacaccggaggccgatgtaactccgactccactagggacgactctgtccggtcctctgacggatgaaggacgacttcgaccgaccgtccaccggacacttctggtaggtgtgacggttaccgaggttgaagtgaccccggtgtcactcccgacggacgaccacccgaccgtagttcgtcctcaaaccgtaggggatgttgggggtcagggtcccccaccaccggaggtacttgttcctcgacttcttctagtaacccgtccactccctggtccgactcgtggacttctgtcgacacgtctaccgacacaagtaggtgttgaagttctccttccccccgtagcccccgatgaggcgacccctctcctaacacctgtagtaacggtgtctgtaggtctggttcctcgaggtcttcgtctagtggttctaggtcttgaagtcccacatgatgtccctgaggtccttgggggacaccttcccgggacggttcgacgacaccttccccctcccccgacaccactaggtcctgttgagactgtagttccaccacgggtcctccttccggttctagtagtccctgataccgttcgtctaccgacccctactgacacaccggaggtccgtcctactcctgatttcgggcccgtctagacgacacggaagatcaacggtcggtagacaacaaacggggagggggcacggaaggaactgggaccttccacggtgagggtgacaggaaaggattattttactcctttaacgtagcgtaacagactcatccacagtaagataagaccccccaccccaccccgtcctgtcgttccccctcctaacccttctgttatcgtccgtacgacccctacgccacccgagataccggctagccgcgcggcatgactttacacacccgcaccgaattcccaccctttcttatatattccacccccagaatacatcaaaacatagacaaaacgtcgtcggcggcggcggtactcgtggttgagcaaactaccttcgtaacactcgagtataaactgttgcgcgtacgggggtacccggccccacgcagtcttacactacccgaggtcgtaactaccagcggggcaggacgggcgtttgagatgatggaactggatgctctggcacagaccttgcggcaacctctgacgtcggaggcggcggcgaagtcggcgacgtcggtggcgggcgccctaacactgactgaaacgaaaggactcgggcgaacgtttgtcacgtcgaagggcaagtaggcgggcgctactgttcaactgccgagaaaaccgtgttaacctaagaaactgggcccttgaattacagcaaagagtcgtcgacaacctagacgcggtcgtccaaagacgggacttccgaaggaggggagggttacgccaaattttgtatttattttttggtctgagacaaacctaaacctagttcgttcacagaacgacagaaataaatccccaaaacgcgcgcgccatccgggccctggtcgccagagccagcaactcccaggacacataaaaaaggtcctgcaccatttccactgagacctacaagtctatgtacccgtattcgggcagagaccccacctccatcgtggtgacgtctcgaagtacgacgccccaccacaacatctactaggtcagcatcgtcctcgcgacccgcaccacggatttttacagaaagtcatcgttcgactaacggtccccgtccgggaaccacattcacaaatgtttcgccaattcgaccctacccacgtatgcacccctatactctacgtagaacctgacataaaaatccaaccgatacaagggtcggtatagggaggcccctaagtacaacacgtcttggtggtcgtgtcacataggccacgtgaaccctttaaacagtacatcgaatcttcctttacgcaccttcttgaacctctgcgggaacactggaggttctaaaaggtacgtaagcaggtattactaccgttacccgggtgcccgccgccggacccgcttctataaagaccctagtgattgcagtatcaacacaaggtcctactctagcagtatccggtaaaaatgtttcgcgcccgcctcccacggtctgacgccatattaccaaggtaggccgggtccccgcatcaatgggagtgtctaaacgtaaagggtgcgaaactcaagtctacccccctagtacagatggacgccccgctacttcttttgccaaaggccccatcccctctagtcgacccttctttcgtccaaggactcgtcgacgctgaatggcgtcggccacccgggcatttagtgtggataatggccgacgttgaccatcaattctctcgacgtcgacggcagtagggactcgtccccccggtgaagcaattcgtacagggactgagcgtacaaaagggactggtttaggcggtcttccgcgagcggcgggtcgctatcgtcaagaacgttccttcgtttcaaaaagttgccaaactctggcaggcggcatccgtacgaaaactcgcaaactggttcgtcaaggtccgccagggtgtcgagccagtggacgagatgccgtagagctaggtcgtatagaggagcaaagcgcccaaccccgccgaaagcgacatgccgtcatcagccacgagcaggtctgcccggtcccagtacagaaaggtgcccgcgtcccaggagcagtcgcatcagacccagtgccacttccccacgcgaggcccgacgcgcgaccggtcccacgcgaactccgaccaggacgaccacgacttcgcgacggccagaagcgggacgcgcagccggtccatcgtaaactggtaccacagtatcaggtcggggaggcgccgcaccgggaaccgcgcgtcgaacgggaacctcctccgcggcgtgctccccgtcacgtctgaaaactcccgcatctcgaacccgcgctctttatggctaaggcccctcatccgtaggcgcggcgtccggggcgtctgccagagcgtaaggtgctcggtccactcgagaccggcaagccccagtttttggtccaaagggggtacgaaaaactacgcaaagaatggagaccaaaggtactcggccacaggtgcgagccactgcttttccgacaggcacaggggcatatgtctgaactctccggacaggagctcgccacaaggcgccaggaggagcatatctttgagcctggtgagactctgtttccgagcgcaggtccggtcgtgcttcctccgattcaccctccccatcgccagcaacaggtgatcccccaggtgagcgaggtcccacacttctgtgtacagcgggagaagccgtagttccttccactaaccaaacatccacatccggtgcactggcccacaaggacttccccccgatattttcccccacccccgcgcaagcaggagtgagagaaggcgtagcgacagacgctcccggtcgacaaccccactcatgagggagacttttcgcccgtactgaagacgcgattctaacagtcaaaggtttttgctcctcctaaactataagtggaccgggcgccactacggaaactcccaccggcgtaggtagaccagtcttttctgttagaaaaacaacagttcgaaccaccgtttgctgggcatctcccgcaacctgtcgttgaaccgctacctcgcgtcccaaaccaaaaacagcgctagccgcgcgaggaaccggcgctacaaatcgacgtgcataagcgcgcgttgcgtggcggtaagccctttctgccaccacgcgagcagcccgtggtccacgtgcgcggttggcgccaacacgtcccactgttccagttgcgaccaccgatggagaggcgcatccgcgagcaaccaggtcgtctccgccggcgggaacgcgctcgtcttaccgccatcccccagatcgacgcagagcaggccccccagacgcaggtgccatttctggggcccgtcgtccgcgcgcagcttcatcagatagaacgtaggaacgttcagatcgcggacgacggtacgcgcccgccgttcgcgcgcgagcatacccaactcaccccctggggtaccgtaccccacccactcgcgcctccgcatgtacggcgtttacagcatttgcatctccccgagagactcataaggttctatacatcccatcgtagaaggtggcgcctacgaccgcgcgtgcattagcatatcaagcacgctccctcgctcctccagccctggctccaacgatgcccgcccgacgagacgagccttctgatagacggacttctaccgtacactcaacctactataccaacctgcgaccttctgcaacttcgaccgcagacactctggatggcgcagtgcgtgcttcctccgcatcctcagcgcgtcgaacaactggtcgagccgccactggacgtgcagatcccgcgtcatcaggtcccaaaggaactactacagtatgaataggacagggaaaaaaaaggtgtcgagcgccaactcctgtttgagaagcgccagaaaggtcatgagaacctagcctttgggcagccggaggcttgccattctcggatcgtacatcttgaccaactgccggaccatccgcgtcgtagggaaaagatgcccatcgcgcatacggacgcgccggaaggcctcgctccacacccactcgcgtttccacagggactggtactgaaactccatgaccataaacttcagtcacagcagcgtaggcgggacgagggtctcgtttttcaggcacgcgaaaaaccttgcgcctaaaccgtcccgcttccactgtagcaacttctcatagaaagggcgcgctccgtatttcaacgcacactacgccttcccagggccgtggagccttgccaacaattaatggacccgccgctcgtgctagagcagtttcggcaactacaacaccgggtgttacatttcaaggttcttcgcgccctacgggaactaccttccgttaaaaaattcaaggagcatccactcgagaagtcccctcgactcgggcacgagactttcccgggtcagacgttctactcccaaccttcgctgcttactcgaggtgtccagtgcccggtaatcgtaaacgtccaccagcgctttccaggatttgaccgctggataccggtaaaaaagaccccactacgtcatcttccattcgcccagaacaagggtcgccagggtaggttccaagcgccgatccagagcgcgccgtcagtgatctccgagtagaggcggcttgaagtactggtcgtacttcccgtgctcgacgaagggtttccgggggtaggttcatatccagagatgtagcatccactgtttctctgcgagccacgctcctacgctcggctagcccttcttgacctagagggcggtggttaacctcctcaccgataactacaccactttcatcttcagggacgctgcccggcttgtgagcacgaccgaaaacatttttgcacgcgtcatgaccgtcgccacgtgcccgacatgtaggacgtgctccaactggactgctggcgcgtgttccttcgtctcacccttaaactcggggagcggaccgcccaaaccgaccaccagaagatgaagccgacgaacaggaactggcagaccgacgagctcccctcaatgccacctagcctggtggtgcggcgcgctcgggtttcaggtctacaggcgcgcgccgccagcctcgaactactgttgtagcgcgtctaccctcgacaggtaccagacctcgagggcgccgcagtccagtccgccctcgaggacgtccaaatggagcgtatctgcccagtcccgcgcccgatctaggtccactatggattaaaggtccccgaccaaccaccgccgcagctaccgaacgttctccggcgtaggggcgccgcgctgatgccatggcgcgccgcccgccacccggcgcccccacaggaacctactacgtagattttcgccactgcgcccgctcgggggcctccatcccccccgaggcctgggcggccctctcccccgtccccgtgcagccgcggcgcgcgcccgtcctcgaccacgacgcgcgcatccaacgaccgcttgcgctgctgcgccgccaactagaggacttagaccgcggagacgcacttctgctgcccgggccactcgaacttggactttctctcaagctgtcttagttaaagccacagcaactgccgccggaccgcgttttagaggacgtgcagaggactcaacagaactatccgctagagccggtacttgacgagctagagaaggaggacctctagaggcgcaggccgagcgaggtgccaccgccgctccagcaacctttacgcccggtactcgacgctcttccgcaactccggagggagcaaggtctgcgccgacatctggtgcgggggaagccgtagcgcccgcgcgtactggtggacgcgctctaactcgaggtgcacggcccgcttctgccgcatcaaagcgtccgcgactttctccatcaactcccaccaccgccacacaagacggtgcttcttcatgtattgggtcgcagcgttgcacctaagcaactatagggggttccggagttccgcgaggtaccggagcatcttcaggtgccgcttcaactttttgaccctcaacgcgcggctgtgccaattgaggaggaggtcttctgcctactcgagccgctgtcacagcgcgtggagcgcgagtttccgatgtccccggagaagaagaagaagttagaggagaaggtattcccggaggggaagaagaagaagaccgccgccaccccctcccccctgtgccgccgctgctgccgcgtggccctccgccagctgtttcgcgagctagtagaggggcgccgctgccgcgtaccagagccactgccgcgccggcaagagcgcccccgcgtcaaccttctgcggcgggcagtacagggccaatacccaaccgccccccgacggtacgccgtccctatgccgcgattgctacgtagagttgttaacaacacatccatgaggcggcggctccctggactcgctcaggcgtagctggcctagccttttggagagctctttccgcagattggtcagtgtcagcgttccatccgactcgtggcaccgcccgccgtcgcccgccgccagccccaacaaagaccgcctccacgacgactactacattaatttcatccgccagaactctgccgcctaccagctgtcttcgtggtacaggaacccaggccggacgacttacgcgtccgccagccggtacggggtccgaagcaaaactgtagccgcgtccagaaacatcatcagaacgtactcggaaagatggccgtgaagaagaagaggaaggagaacaggacgtagagaacgtagatagcgacgccgccgccgcctcaaaccggcatccaccgcgggagaaggagggtacgcacactggggcttcggggagtagccgacttcgtcccgatccagccgctgttgcgcgagccgattataccggacgacgtggacgcactcccatctgaccttcagtaggtacaggtgtttcgccaccatacgcgggcacaactaccacattcacgtcaaccggtattgcctggtcaattgccagaccactgggccgacgctctcgagccacatggactctgcgctcattcgggagctcagtttatgcatcagcaacgttcaggcgtggtccatgaccatagggtggtttttcacgccgccgccgaccgccatctccccggtcgcatcccaccggccccgaggcccccgctctagaaggttgtattccgctactataggcatctacatggacctgtaggtccactacggccgccgccaccacctccgcgcgcctttcagcgcctgcgccaaggtctacaacgcgtcgccgtttttcacgaggtaccagccctgcgagaccggccagtccgcgcgcgttagcaactgcgagatctggcacgttttcctctcggacattcgcccgtgagaaggcaccagaccacctatttaagcgttcccatagtaccgcctgctggccccaagctcggggcataggccggcaggcggcactaggtacgccaatggcgggcgcacagcttgggtccacacgctgcagtctgttgccccctcacgaggaaaaccgaaggaaggtccgcgccgccgacgacgcgatcgaaaaaaccggtgaccggcgcgcgtcgcattcgccaatccgacctttcgctttcgtaattcaccgagcgagggacatcggcctcccaataaaaggttcccaactcagcgccctgggggccaagctcagagcctggccggcctgacgccgcttgcccccaaacggaggggcagtacgttctggggcgaacgtttaaggaggcctttgtccctgctcggggaaaaaacgaaaagggtctacgtaggccacgacgccgtctacgcggggggaggagtcgtcgccgttctcgttctcgtcgccgtctgtacgtcccgtgggaggggaggaggatggcgcagtcctccccgctgtaggcgccaactgcgccgtcgtctaccactaatgcttgggggcgccgcggcccgggccgtgatggacctgaacctcctcccgctcccggaccgcgccgatcctcgcgggagaggactcgccgtgggttcccacgtcgacttcgcactatgcgcactccgcatgcacggcgccgtcttggacaaagcgctggcgctccctctcctcgggctcctctacgccctagctttcaaggtgcgtcccgcgctcgacgccgtaccggacttagcgctcgccaacgacgcgctcctcctgaaactcgggctgcgcgcttggccctaatcagggcgcgcgcgtgtgcaccgccggcggctggaccattggcgtatgctcgtctgccacttggtcctctaattgaaagttttttcgaaattgttggtgcacgcatgcgaacaccgcgcgctcctccaccgatatcctgactacgtagacaccctgaaacattcgcgcgacctcgttttgggtttatcgttcggcgagtaccgcgtcgacaaggaatatcacgtcgtgtcgtccctgttgctccgtaagtccctacgcgacgatttgtatcatctcgggctcccggcgaccgacgagctaaactatttgtaggacgtctcgtatcaccacgtcctcgcgtcgaactcggaccgactgttccaccggcggtagttgataaggtacgaatcggacccgttcaaaatgcgggcgttctatatggtatggggaatgcaagggtatctgttcctccatttctagctccccaagatgtacgcgtaccgcgacttccacgaatggaactcgctgctggacccgcaaatagcgttgctcgcgtaggtgttccggcactcgcactcggccgccgcgctcgagtcgctggcgctcgactacgtgtcggacgtttcccgggaccgaccgtgcccgtcgccgctatctctccggctcaggatgaaactgcgcccgcgactggacgcgacccggggttcggctgcgcgggacctccgtcgaccccggcctggacccgaccgccaccgtgggcgcgcgcgaccgttgcagccgccgcacctccttatactgctcctgctactcatgctcggtctcctgccgctcatgattcgccactacaaagactagtctactacgttctgcgttgcctgggccgccacgcccgccgcgacgtctcggtcggcaggccggaattgaggtgcctgctgaccgcggtccagtacctggcgtagtacagcgactgacgcgcgttaggactgcgcaaggccgtcgtcggcgtccggttggccgagaggcgttaagaccttcgccaccagggccgcgcgcgtttggggtgcgtgctcttccacgaccgctagcatttgcgcgaccggcttttgtcccggtaggccgggctgctccggccggaccagatgctgcgcgacgaagtcgcgcaccgagcaatgttgtcgccgttgcacgtctggttggacctggccgaccaccccctacacgcgctccggcaccgcgtcgcactcgcgcgcgtcgtcgtcccgttggacccgaggtaccaacgtgatttgcggaaggactcatgtgtcgggcggttgcacggcgcccctgtcctcctgatgtggttgaaacactcgcgtgacgccgattaccactgactctgtggcgtttcactccacatggtcagacccggtctgataaaaaaggtctggtcatctgttccggacgtctggcatttggactcggtccgaaagtttttgaacgtccccgacaccccccacgcccgagggtgtccgctggcgcgctggcacagatcgaacgactgcgggttgagcgcggacaacgacgacgattatcgcgggaagtgcctgtcaccgtcgcacagggccctgtgtatggatccagtgaacgactgtgacatggcgctccggtatccagtccgcgtacacctgctcgtatgaaaggtcctctaatgttcacagtcggcgcgcgaccccgtcctcctgtgcccgtcggacctccgttgggatttgatggacgactggttggccgccgtcttctaggggagcaacgtgtcaaatttgtcgctcctcctcgcgtaaaacgcgatgcacgtcgtctcgcactcggaattggactacgcgctgccccattgcgggtcgcaccgcgacctgtactggcgcgcgttgtaccttggcccgtacatacggagtttggccggcaaatagttggcggattacctgatgaacgtagcgcgccggcggcacttggggctcataaagtggttacggtagaacttgggcgtgaccgatggcgggggaccaaagatgtggccccctaagctccacgggctcccattgctacctaaggagaccctgctgtatctgctgtcgcacaaaaggggcgttggcgtctgggacgatctcaacgttgtcgcgctcgtccgtctccgccgcgacgctttcctttcgaaggcgtccggttcgtcgaacaggctagatccgcgacgccggggcgccagtctacgatcatcgggtaaaggttcgaactatcccagagaatggtcgtgagcgtggtgggcgggcgcggacgacccgctcctcctcatggatttgttgagcgacgacgtcggcgtcgcgctttttttggacggaggccgtaaagggttgttgccctatctctcggatcacctgttctactcatctaccttctgcatgcgcgtcctcgtgtccctgcacggtccgggcgcgggcgggtgggcagcagtttccgtgctggcagtcgccccagaccacaccctcctgctactgagccgtctgctgtcgtcgcaggacctaaaccctccctcaccgttgggcaaacgcgtggaagcggggtccgacccctcttacaaaatttttttttttttcgtactacgttttattttttgagtggttccggtaccgtggctcgcaaccaaaagaacataaggggaatcatacgccgcgcgccgctacatactccttccaggaggagggaggatgctctcacaccactcgcgccgcggtcaccgccgccgcgacccaagagggaagctacgaggggacctgggcggcaaacacggaggcgccatggacgccggatggcccccctctttgtcgtaggcaatgagactcaaccgtggggataagctgtggtgggcacacatggaccacctgttgttcagttgcctacaccgtagggacttgatggtcttgctggtgtcgttgaaagactggtgccagtaagttttgttactgatgtcgggccccctccgttcgtgtgtctggtagttagaactgctggccagcgtgaccccgccgctggacttttggtaggacgtatggttgtacggtttacacttgctcaagtacaaatggttattcaaattccgcgcccactaccacagcgcgaacggatgattcctgttagtccacctcgactttatgctcacccacctcaagtgcgacgggctcccgttgatgaggctctggtactggtatctggaatacttgttgcgctagcacctcgtgatgaactttcacccgtctgtcttgccccaagacctttcgctgtagccccatttcaaactgtgggcgttgaagtctgaccccaaactggggcagtgaccagaacagtacggaccccatatatgtttgcttcggaaggtaggtctgtagtaaaacgacggtcctacgccccacctgaagtgggtgtcggcggactcgttgaacaacccgtaggcgttcgccgttgggaaggtcctcccgaaatcctagtggatgctactagacctcccaccattgtaagggcgtgacaacctacacctgcggatggtccgctcgaactttctactgtggcttgtcccgcccccaccgcgtccgccgtcgttgtcgtcaccgtcgccgcgccttctcttgaggttgcgccgtcggcgccgttacgtcggccacctcctgtacttgctagtacggtaagcgccgctgtggaaacggtgtgcccgactcctcttcgcgcgactccggcttcgtcgccggcttcgacggcgggggcgacgcgttgggctccagctcttcggagtcttctttggccactagtttggggactgtctcctgtcgttctttgcgtcaatgttggattattcgttactgtcgtggaagtgggtcatggcgtcgaccatggaacgtatgttgatgccgctgggagtctggccttaggcgagtacctgggacgaaacgtgaggactgcattggacgccgagcctcgtccagatgaccagcaacggtctgtactacgttctggggcactggaaggcgaggtgcgcggtctagtcgttgaaaggccaccacccgcggctcgacaacgggcacgtgaggttctcgaagatgttgctggtccggcagatgagggttgagtaggcggtcaaatggagagactgggtgcacaagttagcgaaagggctcttggtctaaaaccgcgcgggcggtcgggggtggtagtggtggcagtcacttttgcaaggacgagagtgtctagtgccctgcgatggcgacgcgttgtcgtagcctcctcaggtcgctcactggtaatgactgcggtctgcggcgtggacggggatgcaaatgttccgggacccgtatcagagcggcgcgcaggatagctcggcgtgaaaaactcgttcgtacaggtaggaatatagcgggtcgttattgtgtccgaccccggacgcgaagggttcgttctacaaaccgccccggttcttcgcgaggctggttgtgggtcacgcgcacgcgcccgtgatggcgcgcgggaccccgcgcgtgtttgcgccggcgtgacccgcgtggtggcagctactgcggtagctgcgccaccacctcctccgcgcgttgatgtgcgggtgcggcggtggtcacaggtgtcacctgcgccggtaagtctggcaccacgcgcctcgggccgcgatacgattttacttctctgccgcctccgcgcatcgtgcagcggtggcggcggctgggccgtgacggcgggttgcgcgccgccgccgggacgaattggcgcgtgcagcgtggccggctgcccgccggtacgcccggcgagcttccgaccggcgcccataacagtgacacggggggtccaggtccgctgctcgccggcggcgtcgtcggcgccggtaatcacgatactgagtcccagcgtccccgttgcacataacccacgcgctgagccaatcgccggacgcgcacgggcacgcgtgggcggggggcgcgttgatctaacgttcttttttgatgaatctgagcatgacaacatacataggtcgccgccgccgcgcgttgcttcgatacaggttcgcgttttagtttcttctctacgaggtccagtagcgcggcctctagataccggggggcttcttccttctcgtcctaatgttcggggctttcgatttcgcccagtttttctttttctttctactactactacttgaactgctgctccaccttgacgacgtgcgatggcgcgggtccgctgcccatgtcacctttccagctgcgcattttgcacaaaacgctgggccgtggtggcatcagaaatgcgggccactcgcgaggtgggcgtggatgttcgcgcacatactactccacatgccgctgctcctggacgaactcgtccggttgctcgcggagcccctcaaacggatgcctttcgccgtattcctgtacgaccgcaacggcgacctgctcccgttgggttgtggatcggatttcgggcattgtgacgtcgtccacgacgggcgcgaacgtggcaggcttcttttcgcgccggatttcgcgctcagaccactgaaccgtgggtggcacgtcgactaccatgggttcgcggtcgctgaccttctacagaaccttttttactggcaccttggacccgacctcgggctccaggcgcacgccggttagttcgtccaccgcggccctgacccgcacgtctggcacctgcaagtctatgggtgatggtcatcgtggtcataacggtggcggtgtctcccgtacctctgtgtttgcaggggccaacggagtcgccaccgcctacggcgccacgtccgccagcgacgccggcgcaggttctggagatgcctccacgtttgcctgggcacctacaaagcgcaaagtcggggggccgcgggcgcggcaagctccttcatgccgcggcggtcgcgcgatgacgggcttatacgggatgtaggaaggtaacgcggatgggggccgatagcaccgatgtggatggcggggtcttctgctcgttgatgggctgcggcttggtggtgaccttgggcggcggcggcagcggcagcggtcgggcacgaccggggctaaaggcacgcgtcccaccgagcgcttcctccgtcctgggaccacgacggttgtcgcgcgatggtggggtcgtagcaaattttcggccagaaacaccaagaacgtctataccgggagtggacggcggaggcaaagggccacggccctaaggctccttcttacgtggcatcctccccgtaccggccggtgccggactgcccgccgtacgcagcacgcgtggtggccgccgccgcgcgcagcgtggcagcgtacgcgccgccataggacggggaggaataaggtgactagcggcgccgctaaccgcggcacgggccttaacgtaggcaccggaacgtccgcgtctctgtgactaatttttgttcaacgtacacctttttagttttatttttcagacctgagagtgcgagcgaaccaggacattgataaaacatcttaccttctgtagttgaaacgcagagaccggggcgctgtgccgagcgcgggcaagtaccctttgaccgttctatagccgtggtcgttatactcgccaccgcggaagtcgaccccgagcgacacctcgccgtaatttttaaagccaaggtggcaattcttgataccgtcgttccggaccttgtcgtcgtgtccggtctacgactccctattcaactttctcgttttaaaggttgttttccaccatctaccggaccggagaccgtaatcgccccaccacctggaccggttggtccgtcacgttttattctaattgtcattcgaactaggggcgggagggcatctcctcggaggtggccggcacctctgtcacagaggtctccccgcaccgcttttcgcaggcgcggggctgtcccttctttgagaccactgcgtttatctgctcggagggagcatgctcctccgtgatttcgttccggacgggtggtgggcagggtagcgcgggtaccgatggcctcacgacccggtcgtgtgtgggcattgcgacctggacggaggggggcggctgtgggtcgtctttggacacgacggtccgggctggcggcaacaacattgggcaggatcggcgcgcagggacgcggcgcggcggtcgccaggcgctagcaacgccgggcatcggtcaccgttgaccgtttcgtgtgacttgtcgtagcacccagacccccacgttagggacttcgcggctgctacgaagactatcgattgcacagcatacacacagtacatacgcaggtacagcggcggtctcctcgacgactcggcggcgcgcgggcgaaaggttctaccgatggggaagctactacggcgtcaccagaatgtacgtgtagagcccggtcctgcggagcctcatggactcggggcccgaccacgtcaaacgggcgcggtggctctgcatgaagtcggacttattgttcaaatctttggggtgccaccgcggatgcgtgctgcactggtgtctggccagggtcgcaaactgcgacgccaagtagggacacctggcactcctatgacgcatgagcatgttccgcgccaagtgggatcgacacccactattggcacacgacctgtaccgaaggtgcatgaaactgtaggcgccgcacgacctgtccccgggatgaaaattcgggatgagaccgtgacggatgttgcgggaccgagggttcccacggggtttaggaacgcttaccctacttcgacgatgacgagaactttatttggatcttcttctcctgctactgttgcttctgcttcatctgctcgttcgactcgtcgttttttgagtgcataaacccgtccgcggaataagaccatatttataatgtttcctcccataagtttatccacagcttccagtttgtggatttatacggctattttgtaaagttggacttggagtttatcctcttagagtcaccatgctttgtctttaattagtacgtcgaccctctcaggattttttctgatggggttactttggtacaatgccaagtatacgttttgggtgtttacttttacctcccgttccgtaagaacatttcgttgttttacctttcgatctttcagttcacctttacgttaaaaagagttgatgactccgtcggcgtccgttaccactattgaactgaggatttcaccataacatgtcacttctacatctatatctttggggtctgtgagtataaagaatgtacgggtgataattccttccattgagtgctcttgattacccggttgttagatacgggttgtccggattaatgtaacgaaaatccctgttaaaataaccagattacataatgttgtcgtgcccattatacccacaagaccgcccggttcgtagcgtcaacttacgacaacatctaaacgttctgtctttgtgtctcgaaagtatggtcgaaaacgaactaaggtaaccactatcttggtccatgaaaagatacaccttagtccgacaactgtcgatactaggtctacaatcttaataacttttagtaccttgacttctacttgaaggtttaatgacgaaaggtgaccctccacactaattatgtctctgagaatggttccattttggattttgtccagtccttttacctaccctttttctacgatgtcttaaaagtctatttttactttattctcaacctttattaaaacggtacctttagttagatttacggttggacacctctttaaaggacatgaggttgtatcgcgacataaacgggctgttcgatttcatgtcaggaaggttgcatttttaaagactattgggtttgtggatgctgatgtacttgttcgctcaccaccgagggcccgatcacctgacgatgtaattggaacctcgtgcgaccagggaactgatatacctgttgcagttgggtaaattggtggtggcgttacgaccggacgcgatggcgagttacaacgacccgttaccagcgatacacgggaaggtgtaggtccacggagtcttcaagaaacggtaatttttggaggaagaggacggcccgagtatgtggatgctcaccttgaagtccttcctacaattgtaccaagacgtctcgagggatcctttactggattcccaactgcctcggtcgtaattcaaactatcgtaaacggaaatgcggtggaagaaggggtaccgggtgttgtggcggaggtgcgaactccggtacgaatctttgctgtggttgctggtcaggaaattgctgatagagaggcggcggttgtacgagatgggatatgggcggttgcgatggttgcacgggtataggtaggggagggcgttgacccgccgaaaggcgccgacccggaagtgcgcggaattctgattcctttggggtagtgacccgagcccgatgctgggaataatgtggatgagaccgagatatgggatggatctaccttggaaaatggagttggtgtggaaattcttccaccggtaatggaaactgagaagacagtcgaccggaccgttactggcggacgaatgggggttgctcaaactttaattcgcgagtcaactgcccctcccaatgttgcaacgggtcacattgtactggtttctgaccaaggaccatgtttacgatcgattgatattgtaaccgatggtcccgaagatatagggtctctcgatgttcctggcgtacatgaggaagaaatctttgaaggtcgggtactcggcagtccaccacctactatgatttatgttcctgatggttgtccacccgtaggatgtggttgtgttgttgagacctaaacaaccgatggaacgggggtggtacgcgcttcctgtccggatgggacgattgaaggggataggcgaatatccgttctggcgtcaactgtcgtaatgggtctttttcaaagaaacgctagcgtgggaaaccgcgtagggtaagaggtcattgaaatacaggtacccgcgtgagtgtctggacccggttttggaagagatgcggttgaggcgggtgcgcgatctgtactgaaaactccacctagggtacctgctcgggtgggaagaaatacaaaacaaacttcagaaactgcaccaggcacacgtggtcggcgtggcgccgcagtagctttggcacatggacgcgtgcgggaagagccggccgttgcggtgttgtatttcttcgttcgttgtagttgttgtcgacggcggtacccgaggtcactcgtccttgactttcggtaacagtttctagaaccaacacccggtataaaaaacccgtggatactgttcgcgaaaggtccgaaacaaagaggtgtgttcgagcggacgcggtatcagttatgccggccagcgctctgacccccgcatgtgacctaccggaaacggaccttgggcgtgagtttttgtacgatggagaaactcgggaaaccgaaaagactggtcgctgagttcgtccaaatggtcaaactcatgctcagtgaggacgcggcatcgcggtaacgaagaagggggctggcgacatattgcgaccttttcaggtgggtttcgcatgtccccgggttgagccggcggacacctgataagacgacgtacaaagaggtgcggaaacggttgaccggggtttgagggtacctagtgttggggtggtacttggaataatggccccatgggttgaggtacgagttgtcaggggtccatgtcgggtgggacgcagcgttggtccttgtcgagatgtcgaaggacctcgcggtgagcgggatgaaggcgtcggtgtcacgcgtctaatcctcgcggtgaagaaaaacagtgaactttttgtacatttttattacatgatctctgtgaaagttatttccgtttacgaaaataaacatgtgagagcccactaataaatgggggtgggaacggcagacgcggcaaatttttagtttccccaagacggcgcgtagcgatacgcggtgaccgtccctgtgcaacgctatgaccacaaatcacgaggtgaatttgagtccgtgttggtaggcgccgtcgagccacttcaaaagtgaggtgtccgacgcgtggtagtggttgcgcaaatcgtccagcccgcggctatagaacttcagcgtcaaccccggaggcgggacgcgcgcgctcaacgctatgtgtcccaacgtcgtgaccttgtgatagtcgcggcccaccacgtgcgaccggtcgtgcgagaacagcctctagtctaggcgcaggtccaggaggcgcaacgagtcccgcttgcctcagttgaaaccatcgacggaagggtttttcccgcgcacgggtccgaaactcaacgtgagcgtggcatcaccgtagttttccactggcacgggccagacccgcaatcctatgtcgcggacgtattttcggaactagacgaattttcggtggactcggaaacgcggaagtctcttcttgtacggcgttctgaacggccttttgactaaccggcctgtccggcgcagcacgtgcgtcgtggaacgcagccacaacctctagacgtggtgtaaagccggggtggccaagaagtgctagaaccggaacgatctgacgaggaagtcgcgcgcgacgggcaaaagcgagcagtgtaggtaaagttagtgcacgaggaataaatagtattacgaaggcacatctgtgaattcgagcggaagctagagtcgcgtcgccacgtcggtgttgcgcgtcgggcacccgagcactacgaacatccagtggagacgtttgctgacgtccatgcggacgtccttagcggggtagtagcagtgtttccagaacaacgaccacttccagtcgacgttgggcgccacgaggagcaagtcggtccagaacgtatgccggcggtctcgaaggtgaaccagtccgtcatcaaacttcaagcggaaatctagcaataggtgcaccatgaacaggtagtcgcgcgcgcgtcggaggtacgggaagagggtgcgtctgtgctagccgtgtgagtcgcccaagtagtggcattaaagtgaaaggcgaagcgacccgagaaggagaaggagaacgcaggcgtatggtgcgcggtgacccagcagaagtaagtcggcggcgtgacacgcgaatggaggaaacggtacgaactaatcgtggccacccaacgactttgggtggtaaacatcgcggtgtagaagagaaagaaggagcgacaggtgctaatggagaccactaccgcccgcgagcccgaaccctcttcccgcgaagaaaaagaagaacccgcgttaccggtttaggcggcggctccagctaccggcgcccgacccacacgcgccgtggtcgcgcagaacactactcagaaggagcaggagcctgagctatgcggcggagtaggcgaaaaaacccccgcgggcccctccgccgccgctgcccctgcccctgctgtgcaggaggtaccaaccccctgcagcgcggcgtggcgcaggcgcgagcccccaccaaagcgcgacgaggagaagggctgaccggtaaaggaagaggatatccgtctttttctagtacctcagtcagctcttcttcctgtcggattggcgggggagactcaagcggtggtggcggaggtggctacggcggttgcgcggatggtggaaggggcagctccgtgggggcgaactcctcctccttcactaatagctcgtcctgggtccaaaacattcgcttctgctgctcctggcgagtcatggttgtctcctatttttcgttctggtcctgttgcgtctccgtttgctccttgttcagcccgcccccctgctttccgtaccgctgatggatctacaccctctgctgcacgacaacttcgtagacgtcgcggtcacgcggtaatagacgctgcgcaacgttctcgcgtcgctacacggggagcggtatcgcctacagtcggaacggatgcttgcggtggataagagtggcgcgcatggggggtttgcggttcttttgccgtgtacgctcgggttgggcgcggagttgaagatggggcataaacggcacggtctccacgaacggtggatagtgtagaaaaaggttttgacgttctatggggataggacggcacggttggcgtcggctcgcctgttcgtcgaccggaacgccgtcccgcgacagtatggactatagcggagcgagttgcttcacggtttttagaaactcccagaacctgcgctgctcttcgcgcgccgtttgcgagacgttgtccttttgtcgcttttactttcagtgagacctcacaaccaccttgagctcccactgttgcgcgcggatcggcatgattttgcgtcgtagctccagtgggtgaaacggatgggccgtgaattggatggggggttccagtactcgtgtcagtactcactcgactagcacgcggcacgcgtcggggacctctccctacgtttaaacgttcttgtttgtctcctcccggatgggcgtcaaccgctgctcgtcgatcgcgcgaccgaagtttgcgcgctcggacggctgaacctcctcgctgcgtttgattactaccggcgtcacgagcaatggcacctcgaactcacgtacgtcgccaagaaacgactgggcctctacgtcgcgttcgatctcctttgtaacgtgatgtggaaagctgtcccgatgcatgcggtccggacgttctagaggttgcacctcgagacgttggaccagaggatggaaccttaaaacgtgcttttggcggaacccgttttgcacgaagtaaggtgcgagttcccgctccgcgcggcgctgatgcaggcgctgacgcaaatgaataaagatacgatgtggaccgtctgccggtacccgcaaaccgtcgtcacgaacctcctcacgttggagttcctcgacgtctttgacgatttcgttttgaacttcctggatacctgccggaagttgctcgcgaggcaccggcgcgtggaccgcctgtagtaaaaggggcttgcggacgaattttgggacgttgtcccagacggtctgaagtggtcagtttcgtacaacgtcttgaaatccttgaaataggatctcgcgagtccttagaacgggcggtggacgacacgtgaaggatcgctgaaacacgggtaattcatggcgcttacgggaggcggcgaaaccccggtgacgatggaagacgtcgatcggttgatggaacggatggtgagactgtattaccttctgcactcgccactgccagatgacctcacagtgacagcgacgttggatacgtggggcgtggcgagggaccaaacgttaagcgtcgacgaattgctttcagtttaatagccatggaaactcgacgtcccagggagcggactgcttttcaggcgccgaggccccaactttgagtgaggccccgacacctgcagccgaatggaagcgtttaaacatggactcctgatggtgcgggtgctctaatccaagatgcttctggttagggcgggcggattacgcctcgaatggcggacgcagtaatgggtcccggtgtaagaaccggttaacgttcggtagttgtttcgggcggttctcaaagacgatgctttccctgccccccaaatgaacctgggggtcaggccgctcctcgagttgggttaggggggcggcggcgtcgggatagtcgtcgtcggcgcccgggaacgaagggtcctaccgtgggtttttcttcgacgtcgacggcggcggtgggtgcctgctcctccttatgaccctgtcagtccgtctcctccaaaacctgctcctcctcctcctgtactaccttctgaccctctcggatctgctccttcgaaggctccagcttctccacagtctgctttgtggcagtgggagccagcgtaaggggagcggccgcggggtctttagccgttggccaaggtcgtaccgatgttggaggcgaggagtccgcggcggccgtgacgggcaagcggctgggttggcatctaccctgtggtgaccttggtcccggccattcaggttcgtcggcggcggcaatcgggttctcgttgttgtcgcggttccgatggcgagtaccgcgcccgtgttcttgcggtatcaacgaacgaacgttctgacacccccgttgtagaggaagcgggcggcgaaagaagagatggtagtgccgcaccggaagggggcattgtaggacgtaatgatggcagtagagatgtcgggtatgacgtggccgccgtcgccgtcgttgtcgtcgccggtgtgtcttcgtttccgctggcctatcgttctgagactgtttcgggttctttaggtgtcgccgccgtcgtcgtcctcctcctcgcgacgcagaccgcgggttgcttgggcatagctgggcgctcgaatctttgtcctaaaaagggtgagacatacgatataaagttgtctcgtccccggttcttgttctcgacttttattttttgtccagagacgctagggagtgggcgtcgacggacatagtgttttcgcttctagtcgaagccgcgtgcgaccttctgcgcctccgagagaagtcatttatgacgcgcgactgagaattcctgatcaaagcgcgggaaagagtttaaattcgcgcttttgatgcagtagaggtcgccggtgtgggccgcggtcgtggacaacagtcgcggtaatactcgttcctttaagggtgcgggatgtacacctcaatggtcggtgtttaccctgaacgccgacctcgacgggttctgatgagttgggcttatttgatgtactcgcgccctggggtgtactatagggcccagttgccttatgcgcgggtggctttggcttaagaggaccttgtccgccgataatggtggtgtggagcattattggaattaggggcatcaaccgggcgacgggaccacatggtcctttcagggcgagggtggtgacaccatgaagggtctctgcgggtccggcttcaagtctactgattgagtccccgcgtcgaacgcccgccgaaagcagtgtcccacgccagcgggcccgtcccatattgagtggactgttagtctcccgctccataagtcgagttgctgctcagccactcgaggagcgaaccagaggcaggcctgccctgtaaagtctagccgccgcggccggcgagaagtaagtgcggagcagtccgttaggattgagacgtctggagcaggagactcggcgcgagacctccgtaaccttgagacgttaaataactcctcaaacacggtagccagatgaaattggggaagagccctggagggccggtgataggcctagttaaataaggattgaaactgcgccatttcctgagccgcctgccgatgctgacttacaattcacctctccgtctcgttgacgcggactttgtggaccaggtgacagcggcggtgttcacgaaacgggcgctgaggccactcaaaacgatgaaacttaacgggctcctagtatagctcccgggccgcgtgccgcaggccgaatggcgggtccctctcgaacgggcatcggactaagccctcaaatgggtcgcgggggacgatcaactcgccctgtcccctgggacacaagagtgacactaaacgttgacaggattgggacctaatgtagttctagaaacaacggtagagacacgactcatattatttatgtctttaattttatatgaccccgaggatagcggtaggacatttgcggtggcagaagtgggcgggttcgtttggttccgcttggaatggaccatgaaaattgtagagagggagacactaaatgttgtcaaagttgggtctgcctcactcagatgctctcttggagaggctcgagtcgatgaggtagtcttttttgtggtgggaggaatggacggcccttgcatgctcacgcagtggccggcgacgtggtgtggatggcggactggcatttggtctgaaaaaggcctgtctggagttattgagacaaatggtcttgtcctccactcgaatcttttgggaatcccataatccggtttccgcgtcgatgacaccccaaatacttgttaagttcgttgagatgcccgataagattaagtccaaagagatcttagccccaaccccaataagagacagaacactaagagaaataagaatatgattgcgaagagacggattccgagcggcggacgacacacgtgtaaacgtaaataacagtcgaaaaatttgcgaccccagcggtgggttctactaatccatgtattaggatccaaatgagtgggaacgcagtcgggtgccatggtgggttttccacctaaaattcctcggtcggacattacaatgtaagcgtcgacttcgattactcacgtggtgagaatattttacgtggtgtcttgtacttttcgacgaataagcggtgtttttgttttaaccgttcatacgacaaatacgataaaccgtcggtccactgtgatgtctcatattacaatgtcaaaaggtcccattttcagtattttgaaaatacatatgaaaaggtaaaatactttacacgctgtaatggtacatgtactcgtttgtcatattcaacaccgggggtgttttaacacaccttttgtgaccgtgaaagacgacgtgacgatacgattaatgtcacgagcgaaaccagacatgggatgagatataatttatgttttcgtctgcgtcgaaataactccttttcttttacggaattaaatgattcaatgtttcgattacagtggtgattgacgaaatgagcgacgaacgttttgtttaagtttttcaatcgtaatattaatcttatcctaaatttggggggccagtaaaggacgagttatggtaaggggacttgttaactgagatacaccctatacgaggtcgcgatgttggaacttcagtccgaaggacctacagtcgtagactgaaaccggtcgtggacagggcgcctaaacaaggtcaggttgatgtcgctgggtgggattgtctctactggttgtgttggttgcgccggcggcgatggcctgaatgtagatggtgtttatgtggggttcaaagacggaaacagttattgaccctattgaacccgtacaccaccaagaggtatcgcgaatacaaacatacggaataataatacaccgagtagacgacggatttcgcgtttgcgcgggctggtgggtagatatcagggtagtaacacgatgtgggtttgttactaccttaggtatctaacctgcctgactttgtgtacaagaaaagagaatgtcatactaatttactctgtactaaggagctcaaaaatataatgactgggaacaacgcgaaaaaacacgcacgaggtgtaaccgacgccaaagagtgtagcttcatctgacgtaaggtcggaagtgtcagataaacgaaatgcctaaacagtgggagtgcgagtagacgtcggagtagtgacaccagtagcggaaataggtcacgtaactgacccagacacacgcgaaacgtatagagtctgtggtaggggtcatgtccctgtcctgatatcgactcgaagaatcttaagaaattaatactttaaatgacactgaaaagacgactaataaacgtgggatagacgcaaaacaaggggctggaggttcggagtttctgtatatagtacgtctaagtgagcatataccttataaggttcaacgatgttacttttttcgctagaaaggcttcggaccaatatacgttagtagagacaataccacaagacgtcatggtagaatcgggatcgatatatagggatggaactgtaaccgaccttgcgttatctacggtacttggtgggttgaaaggggcgcgggcgatacgaaggtgacgttgttcaacaacggccgccgaaacagggtcggttagtcggagcgggtggaagagggtgggggtgactttagtcgatgaaattagattgtcctcctctactgactgtgggatctagatctttacctgccttaataatgtctcgtcgcggacgatctttctgcgtcccgtcgccggctcgttgtcgcgtacttagttctcgaggttctgtaccaattgaacgtggtcacgttttccccatagaaaacagagcatttcgtccggtttcagtggatgctgtcattatggtggcctgtggcggaatcgatgttcaacggttggttcgcagtctttaaccaccagtaccaccctcttttcgggtaatggtattgagtcgtgagccatctttggcttccgacgtaagtgagtggaacagttcctggactcctagagacgtgggaataattctgggacacgccagagtttctagaataagggaaattgattatttttttttattatttcgtagtgaatgaattttagtcaatcgtttaaagacaggtcaaataagtcgtcgtggaggaacgggaggagggtcgagaccataacgtcgaaggaggaccgacgtttgaaagaggtgttagatttaccttacagtcaaaggaggacaaggacaggtaggcgtgggtgatagaagtacaacaacgtctacttcgcgcgttctggcagacttctatggaagttggggcacataggtatactgtgcctttggccaggaggttgacacggaaaagaatgaggagggaaacatagggggttacccaaagttctctcagggggaccccatgagagaaacgcggataggcttggagatcaatggaggttaccgtacgaacgcgagttttacccgttgccggagagagacctgctccggccgttggaatggagggttttacattggtgacactcgggtggagagtttttttggttcagtttgtatttggacctttatagacgtggggagtgtcaatggagtcttcgggattgacaccgacggcggcgtggagattaccagcgcccgttgtgtgagtggtacgttagtgtccggggcgattggcacgtgctgaggtttgaatcgtaacggtgggttcctggggagtgtcacagtcttcctttcgatcgggacgtttgtagtccgggggagtggtggtggctatcgtcatgggaatgatagtgacggagtgggggagattgatgacggtgaccatcgaacccgtaactgaactttctcgggtaaatatgtgttttaccttttgatcctgatttcatgccccgaggaaacgtacattgtctgctggatttgtgaaactggcatcgttgaccaggtccacactgataattattatgaaggaacgtttgatttcaatgacctcggaacccaaaactaagtgttccgttatacgttgaattacatcgtcctcctgattcctaactaagagttttgtctgcggaatatgaactacaatcaataggcaaactacgagttttggttgatttagattctgatcctgtcccgggagaaaaatatttgagtcgggtgttgaacctataattgatgttgtttccggaaatgaacaaatgtcgaagtttgttaaggtttttcgaactccaattggattcgtgacggttccccaactacaaactgcgatgtcggtatcggtaattacgtcctctacccgaacttaaaccaagtggattacgtggtttgtgtttaggggagttttgtttttaaccggtaccggatcttaaactaagtttgttccgataccaaggatttgatccttgaccggaatcaaaactgtcgtgtccacggtaatgtcatcctttgtttttattactattcgattgaaacacctggtgtggtcgaggtagaggattgacatctgatttacgtctctttctacgatttgagtgaaaccagaattgttttacaccgtcagtttatgaacgatgtcaaagtcaaaaccgacaatttccgtcaaaccgaggttatagaccttgtcaagtttcacgagtagaataatattctaaactgcttttacctcacgatgatttgttaaggaaggacctgggtcttataaccttgaaatctttacctctagaatgacttccgtgtcggatatgtttgcgacaacctaaatacggattggatagtcgaataggttttagagtgccattttgacggttttcattgtaacagtcagttcaaatgaatttgcctctgttttgatttggacattgtgattggtaatgtgatttgccatgtgtcctttgtcctctgtgttgaggttcacgtatgagatacagtaaaagtaccctgaccagaccggtgttgatgtaattactttataaacggtgtaggagaatgtgaaaaagtatgtaacgggttcttatttcttagcaaacacaatacaaagttgcacaaataaaaagttaacgtcttttaaagttcagtaaaaagtaagtcatcatatcggggtggtggtgtatcgaatatgtctagtggcatggaattagtttgagtgtcttgggatcataagttggacggtggagggagggttgtgtgtctcatgtgtcaggaaagaggggccgaccggaatttttcgtagtatagtacccattgtctgtataagaatccacaatataaggtgtgccaaaggacagctcggtttgcgagtagtcactataattatttgaggggcccgtcgagtgaattcaagtacagcgacaggtcgacgactcggtgtccgacgacaggttgaacgccaacgaattgcccgccgcttcctcttcaggtgcggatgtacccccatctcagtattagcacgtagtcctatcccgccaccacgacgtcgtcgcgcgcttatttgacgacggcggcggcgaggcaggacgtccttatgttgtaccgtcaccagaggagtcgctactaagcgtggcgggcgtcgtattccgcggaacaggaggcccgtgtcgtcgcgtgggactagagtgaatttagtcgtgtcattgacgtcgtgtcgtggtgttataacaagttttagggtgtcacgttccgcgacataggtttcgagtaccgcccctggtgtcttgggtgcaccggtagtatggtgttcgcgtccatctaattcaccgctggggagtatttgtgcgacctgtatttgtaatggagaaaaccgtacaacattaagtggtggagggccatggtatatttggagactaatttgtaccgcggtaggtggtggtaggatttggtcgaccggttttggacgggcggccgatatgtgacgtcccttggccctgaccttgttactgtcacctctcgggtcctgagcattggtacctagtagtacgagcagtactatagttacaaccgtgttgtgtccgtgtgcacgtatgtgaaggagtcctaatgttcgaggagggcgcaatcttggtatagggtcccttgttgggtaaggacttagtcgcatttagggtgtgacgtcccttctggagcgtgcattgagtgcaacacgtaacagtttcacaatgtaagcccgtcgtcgcctactaggaggtcataccatcgcgcccaaagacagagttttcctccatctgctagggatgacatgcctcacgcggctctgttggctctagcacaaccagcatcacagtacggtttaccttgcggcctgcatcagtataaaggacttcgttttggtccacgcccgcactgtttgtctagacgcagaggccagagcggcgaatctagcgagacacatcatcaacatcatataggtgagagagtttcgtaggtccgcgggggaccgaagcccaagatacatttgaggaagtacgcggcgacgggactattgtaggtggtggcgtcttattcggtgtgggtcggttggatgtgtaagcaagacgctcagtgtgtgccctcctcgcccttctcgaccttcttggtacaaaaaaaaaaataaggttttctaataggttttggagttttacttctagataattcacttgcgcgaggggaggccaccgcaccagtttgagatgtcggtttcttgtctattaccgtaaacattctacaacgtgttaccgaaggttttccgtttgccgggagtgcaggttcacctgcatttccgatttgggaagtcccacttagaggagatatttgtaaggtcgtggaagttggtacgggtttattaagagtagagcggtggaagagttatatagagattcgtttagggcttataattcaggccggtaacatttttagacgaggtctcgcgggaggtggaagtcggagttcgtcgcttagtactaacgtttttaagtccaaggagtgtctggacatattctaagttttcgccttgtaattgtttttatggcgctagggcatccagggaagcgtcccggtcgacttgtattagcacgtccagacgtgcctggtcgcgccggtgaaggggcggtccttggtactgttttcttgggtgtgactaatactgtgcgtatgagcctcgatacgattggtcgcatcggggctacattcgaacaacgtacccgccgctatattttacgttccacgacgagttttttagtccgtttcggagcgcgttttttctttcgtgtagcatcagtacgagtacgtctatttccgtccattcgaggccttggtggtgtctttttctgtggtaaaaagagagtttgtacagacgcccaaagacgtatttgtgttttattttattgtttttttgtaaatttgtaatcttcggacagaatgttgtcctttttgttgggaatattcgtattctgcctgatgccggtacggccgcactggcatttttttgaccagtggcactaatttttcgtggtggctgtcgaggagccagtacaggcctcagtattacattctgagccatttgtgtagtccaactaagtgtagccagtcacgatttttcgctggctttatcgggcccccttatgtatgggcgtccgcatctctgttgtaatgtcgggggtatcctccatattgttttaattatcctctctttttgtgtatttgtggactttttgggaggacggatccgttttatcgtgggagggcgaggtcttgttgtatgtcgcgaaggtgtcgccgtcggtattgtcagtcggaatggtcattttttcttttggataatttttttgtggtgagctgtgccgtggtcgagttagtcagtgtcacattttttcccggttcacgtctcgctcatatatatcctgattttttactgcattgccaatttcaggtgttttttgtgggtcttttggcgtgcgcttggatgcgggtctttgctttcggttttttgggtgttgaaggagtttagcagtgaaggcaaaagggtgcaatgcagtgaagggtaaaattcttttgatgttaagggttgtgtatgttcaatgaggcgggattttggatgcagtgggcggggcaagggtgcggggcgcggtgcagtgtttgaggtgggggagtaatagtataaccgaagttaggttttattccatataataactactacaattaattcttaagcctagacgctgcgctccgacctaccggaaggggtaatactaagaagagcgaaggccgccgtagccctacgggcgcaacgtccggtacgacaggtccgtccatctactgctggtagtccctgtcgaagttccggtcgttttccggtccttggcatttttccggcgcaacgaccgcaaaaaggtatccgaggcggggggactgctcgtagtgtttttagctgcgagttcagtctccaccgctttgggctgtcctgatatttctatggtccgcaaagggggaccttcgagggagcacgcgagaggacaaggctgggacggcgaatggcctatggacaggcggaaagagggaagcccttcgcaccgcgaaagagtatcgagtgcgacatccatagagtcaagccacatccagcaagcgaggttcgacccgacacacgtgcttggggggcaagtcgggctggcgacgcggaataggccattgatagcagaactcaggttgggccattctgtgctgaatagcggtgaccgtcgtcggtgaccattgtcctaatcgtctcgctccatacatccgccacgatgtctcaagaacttcaccaccggattgatgccgatgtgatcttcctgtcataaaccatagacgcgagacgacttcggtcaatggaagcctttttctcaaccatcgagaactaggccgtttgtttggtggcgaccatcgccaccaaaaaaacaaacgttcgtcgtctaatgcgcgtctttttttcctagagttcttctaggaaactagaaaagatgccccagactgcgagtcaccttgcttttgagtgcaattccctaaaaccagtactctaatagtttttcctagaagtggatctaggaaaatttagttagatttcatatatactcatttgaaccagactgtcaatggttacgaattagtcactccgtggatagagtcgctagacagataaagcaagtaggtatcaacggactgaggggcagcacatctattgatgctatgccctcccgaatggtagaccggggtcacgacgttactatggcgctctgggtgcgagtggccgaggtctaaatagtcgttatttggtcggtcggccttcccggctcgcgtcttcaccaggacgttgaaataggcggaggtaggtcagataattaacaacggcccttcgatctcattcatcaagcggtcaattatcaaacgcgttgcaacaacggtaacgatgtccgtagcaccacagtgcgagcagcaaaccataccgaagtaagtcgaggccaagggttgctagttccgctcaatgtactagggggtacaacacgttttttcgccaatcgaggaagccaggaggctagcaacagtcttcattcaaccggcgtcacaatagtgagtaccaataccgtcgtgacgtattaagagaatgacagtacggtaggcattctacgaaaagacactgaccactcatgagttggttcagtaagactcttatcacatacgccgctggctcaacgagaacgggccgcagttgtgccctattatggcgcggtgtatcgtcttgaaattttcacgagtagtaaccttttgcaagaagccccgcttttgagagttcctagaatggcgacaactctaggtcaagctacattgggtgagcacgtgggttgactagaagtcgtagaaaatgaaagtggtcgcaaagacccactcgtttttgtccttccgttttacggcgttttttcccttattcccgctgtgcctttacaacttatgagtatgagaaggaaaaagttataataacttcgtaaatagtcccaataacagagtactcgcctatgtataaacttacataaatctttttatttgtttatccccaaggcgcgtgtaaaggggcttttcacggtggactgcagattctttggtaataatagtactgtaattggatatttttatccgcatagtgctccgggaaagcagaagttcttaacctaggcttaagaattaaagaattaatt

SEQ ID NO: 30 - DNA, 36620 bp, Artificial Sequence
  Codon optimized DNA encoding pMRKAd5HIV-1 nef, coding
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattataggcggccgcgatccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgagatctgccaccatggccggcaagtggtccaagaggtccgtgcccggctggtccaccgtgagggagaggatgaggagggccgagcccgccgccgacagggtgaggaggaccgagcccgccgcagtgggcgtgggcgccgtgtccagggacctggagaagcacggcgccatcacctcctccaacaccgccgccaccaacgccgactgcgcctggctggaggcccaggaggacgaggaggtgggcttccccgtgaggccccaggtgcccctgaggcccatgacctacaagggcgccgtggacctgtcccacttcctgaaggagaagggcggcctggagggcctgatccactcccagaagaggcaggacatcctggacctgtgggtgtaccacacccagggctacttccccgactggcagaactacacccccggccccggcatcaggttccccctgaccttcggctggtgcttcaagctggtgcccgtggagcccgagaaggtggaggaggccaacgagggcgagaacaactgcgccgcccaccccatgtcccagcacggcatcgaggaccccgagaaggaggtgctggagtggaggttcgactccaagctggccttccaccacgtggccagggagctgcaccccgagtactacaaggactgctaaagcccgggcagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccgatcggcgcgccgtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaaacagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaacgcaatagatgccatgaaccacccaactttccccgcgcccgctatgcttccactgcaacaagttgttgccggcggctttgtcccagccaatcagcctcgcccaccttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctcgtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaagaattcggatctgcgacgcgaggctggatggccttccccattatgattcttctcgcttccggcggcatcgggatgcccgcgttgcaggccatgctgtccaggcaggtagatgacgaccatcagggacagcttcaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggatccgaattcttaatttcttaattaa

SEQ ID NO: 31 - DNA, 36620 bp, Artificial Sequence
  Codon optimized DNA encoding pMRKAd5HIV-1 nef, noncoding
gtagtagttattatatggaataaaacctaacttcggttatactattactcccccacctcaaacactgcaccgcgccccgcacccttgccccgcccactgcatcatcacaccgccttcacactacaacgttcacaccgccttgtgtacattcgctgcctacaccgttttcactgcaaaaaccacacgcggccacatgtgtccttcactgttaaaagcgcgccaaaatccgcctacaacatcatttaaacccgcattggctcattctaaaccggtaaaagcgcccttttgacttattctccttcactttagacttattaaaacacaatgagtatcgcgcattataaacagatcccggcgcccctgaaactggcaaatgcacctctgagcgggtccacaaaaagagtccacaaaaggcgcaaggcccagtttcaaccgcaaaataataatatccgccggcgctaggtaacgtatgcaacataggtatagtattatacatgtaaatataaccgagtacaggttgtaatggcggtacaactgtaactaataactgatcaataattatcattagttaatgccccagtaatcaagtatcgggtatatacctcaaggcgcaatgtattgaatgccatttaccgggcggaccgactggcgggttgctgggggcgggtaactgcagttattactgcatacaagggtatcattgcggttatccctgaaaggtaactgcagttacccacctcataaatgccatttgacgggtgaaccgtcatgtagttcacatagtatacggttcatgcgggggataactgcagttactgccatttaccgggcggaccgtaatacgggtcatgtactggaataccctgaaaggatgaaccgtcatgtagatgcataatcagtagcgataatggtaccactacgccaaaaccgtcatgtagttacccgcacctatcgccaaactgagtgcccctaaaggttcagaggtggggtaactgcagttaccctcaaacaaaaccgtggttttagttgccctgaaaggttttacagcattgttgaggcggggtaactgcgtttacccgccatccgcacatgccaccctccagatatattcgtctcgagcaaatcacttggcagtctagcggacctctgcggtaggtgcgacaaaactggaggtatcttctgtggccctggctaggtcggaggcgccggcccttgccacgtaaccttgcgcctaaggggcacggttctcactctagacggtggtaccggccgttcaccaggttctccaggcacgggccgaccaggtggcactccctctcctactcctcccggctcgggcggcggctgtcccactcctcctggctcgggcggcgtcacccgcacccgcggcacaggtccctggacctcttcgtgccgcggtagtggaggaggttgtggcggcggtggttgcggctgacgcggaccgacctccgggtcctcctgctcctccacccgaaggggcactccggggtccacggggactccgggtactggatgttcccgcggcacctggacagggtgaaggacttcctcttcccgccggacctcccggactaggtgagggtcttctccgtcctgtaggacctggacacccacatggtgtgggtcccgatgaaggggctgaccgtcttgatgtgggggccggggccgtagtccaagggggactggaagccgaccacgaagttcgaccacgggcacctcgggctcttccacctcctccggttgctcccgctcttgttgacgcggcgggtggggtacagggtcgtgccgtagctcctggggctcttcctccacgacctcacctccaagctgaggttcgaccggaaggtggtgcaccggtccctcgacgtggggctcatgatgttcctgacgatttcgggcccgtctagacgacacggaagatcaacggtcggtagacaacaaacggggagggggcacggaaggaactgggaccttccacggtgagggtgacaggaaaggattattttactcctttaacgtagcgtaacagactcatccacagtaagataagaccccccaccccaccccgtcctgtcgttccccctcctaacccttctgttatcgtccgtacgacccctacgccacccgagataccggctagccgcgcggcatgactttacacacccgcaccgaattcccaccctttcttatatattccacccccagaatacatcaaaacatagacaaaacgtcgtcggcggcggcggtactcgtggttgagcaaactaccttcgtaacactcgagtataaactgttgcgcgtacgggggtacccggccccacgcagtcttacactacccgaggtcgtaactaccagcggggcaggacgggcgtttgagatgatggaactggatgctctggcacagaccttgcggcaacctctgacgtcggaggcggcggcgaagtcggcgacgtcggtggcgggcgccctaacactgactgaaacgaaaggactcgggcgaacgtttgtcacgtcgaagggcaagtaggcgggcgctactgttcaactgccgagaaaaccgtgttaacctaagaaactgggcccttgaattacagcaaagagtcgtcgacaacctagacgcggtcgtccaaagacgggacttccgaaggaggggagggttacgccaaattttgtatttattttttggtctgagacaaacctaaacctagttcgttcacagaacgacagaaataaatccccaaaacgcgcgcgccatccgggccctggtcgccagagccagcaactcccaggacacataaaaaaggtcctgcaccatttccactgagacctacaagtctatgtacccgtattcgggcagagaccccacctccatcgtggtgacgtctcgaagtacgacgccccaccacaacatctactaggtcagcatcgtcctcgcgacccgcaccacggatttttacagaaagtcatcgttcgactaacggtccccgtccgggaaccacattcacaaatgtttcgccaattcgaccctacccacgtatgcacccctatactctacgtagaacctgacataaaaatccaaccgatacaagggtcggtatagggaggcccctaagtacaacacgtcttggtggtcgtgtcacataggccacgtgaaccctttaaacagtacatcgaatcttcctttacgcaccttcttgaacctctgcgggaacactggaggttctaaaaggtacgtaagcaggtattactaccgttacccgggtgcccgccgccggacccgcttctataaagaccctagtgattgcagtatcaacacaaggtcctactctagcagtatccggtaaaaatgtttcgcgcccgcctcccacggtctgacgccatattaccaaggtaggccgggtccccgcatcaatgggagtgtctaaacgtaaagggtgcgaaactcaagtctacccccctagtacagatggacgccccgctacttcttttgccaaaggccccatcccctctagtcgacccttctttcgtccaaggactcgtcgacgctgaatggcgtcggccacccgggcatttagtgtggataatggccgacgttgaccatcaattctctcgacgtcgacggcagtagggactcgtccccccggtgaagcaattcgtacagggactgagcgtacaaaagggactggtttaggcggtcttccgcgagcggcgggtcgctatcgtcaagaacgttccttcgtttcaaaaagttgccaaactctggcaggcggcatccgtacgaaaactcgcaaactggttcgtcaaggtccgccagggtgtcgagccagtggacgagatgccgtagagctaggtcgtatagaggagcaaagcgcccaaccccgccgaaagcgacatgccgtcatcagccacgagcaggtctgcccggtcccagtacagaaaggtgcccgcgtcccaggagcagtcgcatcagacccagtgccacttccccacgcgaggcccgacgcgcgaccggtcccacgcgaactccgaccaggacgaccacgacttcgcgacggccagaagcgggacgcgcagccggtccatcgtaaactggtaccacagtatcaggtcggggaggcgccgcaccgggaaccgcgcgtcgaacgggaacctcctccgcggcgtgctccccgtcacgtctgaaaactcccgcatctcgaacccgcgctctttatggctaaggcccctcatccgtaggcgcggcgtccggggcgtctgccagagcgtaaggtgctcggtccactcgagaccggcaagccccagtttttggtccaaagggggtacgaaaaactacgcaaagaatggagaccaaaggtactcggccacaggtgcgagccactgcttttccgacaggcacaggggcatatgtctgaactctccggacaggagctcgccacaaggcgccaggaggagcatatctttgagcctggtgagactctgtttccgagcgcaggtccggtcgtgcttcctccgattcaccctccccatcgccagcaacaggtgatcccccaggtgagcgaggtcccacacttctgtgtacagcgggagaagccgtagttccttccactaaccaaacatccacatccggtgcactggcccacaaggacttccccccgatattttcccccacccccgcgcaagcaggagtgagagaaggcgtagcgacagacgctcccggtcgacaaccccactcatgagggagacttttcgcccgtactgaagacgcgattctaacagtcaaaggtttttgctcctcctaaactataagtggaccgggcgccactacggaaactcccaccggcgtaggtagaccagtcttttctgttagaaaaacaacagttcgaaccaccgtttgctgggcatctcccgcaacctgtcgttgaaccgctacctcgcgtcccaaaccaaaaacagcgctagccgcgcgaggaaccggcgctacaaatcgacgtgcataagcgcgcgttgcgtggcggtaagccctttctgccaccacgcgagcagcccgtggtccacgtgcgcggttggcgccaacacgtcccactgttccagttgcgaccaccgatggagaggcgcatccgcgagcaaccaggtcgtctccgccggcgggaacgcgctcgtcttaccgccatcccccagatcgacgcagagcaggccccccagacgcaggtgccatttctggggcccgtcgtccgcgcgcagcttcatcagatagaacgtaggaacgttcagatcgcggacgacggtacgcgcccgccgttcgcgcgcgagcatacccaactcaccccctggggtaccgtaccccacccactcgcgcctccgcatgtacggcgtttacagcatttgcatctccccgagagactcataaggttctatacatcccatcgtagaaggtggcgcctacgaccgcgcgtgcattagcatatcaagcacgctccctcgctcctccagccctggctccaacgatgcccgcccgacgagacgagccttctgatagacggacttctaccgtacactcaacctactataccaacctgcgaccttctgcaacttcgaccgcagacactctggatggcgcagtgcgtgcttcctccgcatcctcagcgcgtcgaacaactggtcgagccgccactggacgtgcagatcccgcgtcatcaggtcccaaaggaactactacagtatgaataggacagggaaaaaaaaggtgtcgagcgccaactcctgtttgagaagcgccagaaaggtcatgagaacctagcctttgggcagccggaggcttgccattctcggatcgtacatcttgaccaactgccggaccatccgcgtcgtagggaaaagatgcccatcgcgcatacggacgcgccggaaggcctcgctccacacccactcgcgtttccacagggactggtactgaaactccatgaccataaacttcagtcacagcagcgtaggcgggacgagggtctcgtttttcaggcacgcgaaaaaccttgcgcctaaaccgtcccgcttccactgtagcaacttctcatagaaagggcgcgctccgtatttcaacgcacactacgccttcccagggccgtggagccttgccaacaattaatggacccgccgctcgtgctagagcagtttcggcaactacaacaccgggtgttacatttcaaggttcttcgcgccctacgggaactaccttccgttaaaaaattcaaggagcatccactcgagaagtcccctcgactcgggcacgagactttcccgggtcagacgttctactcccaaccttcgctgcttactcgaggtgtccagtgcccggtaatcgtaaacgtccaccagcgctttccaggatttgaccgctggataccggtaaaaaagaccccactacgtcatcttccattcgcccagaacaagggtcgccagggtaggttccaagcgccgatccagagcgcgccgtcagtgatctccgagtagaggcggcttgaagtactggtcgtacttcccgtgctcgacgaagggtttccgggggtaggttcatatccagagatgtagcatccactgtttctctgcgagccacgctcctacgctcggctagcccttcttgacctagagggcggtggttaacctcctcaccgataactacaccactttcatcttcagggacgctgcccggcttgtgagcacgaccgaaaacatttttgcacgcgtcatgaccgtcgccacgtgcccgacatgtaggacgtgctccaactggactgctggcgcgtgttccttcgtctcacccttaaactcggggagcggaccgcccaaaccgaccaccagaagatgaagccgacgaacaggaactggcagaccgacgagctcccctcaatgccacctagcctggtggtgcggcgcgctcgggtttcaggtctacaggcgcgcgccgccagcctcgaactactgttgtagcgcgtctaccctcgacaggtaccagacctcgagggcgccgcagtccagtccgccctcgaggacgtccaaatggagcgtatctgcccagtcccgcgcccgatctaggtccactatggattaaaggtccccgaccaaccaccgccgcagctaccgaacgttctccggcgtaggggcgccgcgctgatgccatggcgcgccgcccgccacccggcgcccccacaggaacctactacgtagattttcgccactgcgcccgctcgggggcctccatcccccccgaggcctgggcggccctctcccccgtccccgtgcagccgcggcgcgcgcccgtcctcgaccacgacgcgcgcatccaacgaccgcttgcgctgctgcgccgccaactagaggacttagaccgcggagacgcacttctgctgcccgggccactcgaacttggactttctctcaagctgtcttagttaaagccacagcaactgccgccggaccgcgttttagaggacgtgcagaggactcaacagaactatccgctagagccggtacttgacgagctagagaaggaggacctctagaggcgcaggccgagcgaggtgccaccgccgctccagcaacctttacgcccggtactcgacgctcttccgcaactccggagggagcaaggtctgcgccgacatctggtgcgggggaagccgtagcgcccgcgcgtactggtggacgcgctctaactcgaggtgcacggcccgcttctgccgcatcaaagcgtccgcgactttctccatcaactcccaccaccgccacacaagacggtgcttcttcatgtattgggtcgcagcgttgcacctaagcaactatagggggttccggagttccgcgaggtaccggagcatcttcaggtgccgcttcaactttttgaccctcaacgcgcggctgtgccaattgaggaggaggtcttctgcctactcgagccgctgtcacagcgcgtggagcgcgagtttccgatgtccccggagaagaagaagaagttagaggagaaggtattcccggaggggaagaagaagaagaccgccgccaccccctcccccctgtgccgccgctgctgccgcgtggccctccgccagctgtttcgcgagctagtagaggggcgccgctgccgcgtaccagagccactgccgcgccggcaagagcgcccccgcgtcaaccttctgcggcgggcagtacagggccaatacccaaccgccccccgacggtacgccgtccctatgccgcgattgctacgtagagttgttaacaacacatccatgaggcggcggctccctggactcgctcaggcgtagctggcctagccttttggagagctctttccgcagattggtcagtgtcagcgttccatccgactcgtggcaccgcccgccgtcgcccgccgccagccccaacaaagaccgcctccacgacgactactacattaatttcatccgccagaactctgccgcctaccagctgtcttcgtggtacaggaacccaggccggacgacttacgcgtccgccagccggtacggggtccgaagcaaaactgtagccgcgtccagaaacatcatcagaacgtactcggaaagatggccgtgaagaagaagaggaaggagaacaggacgtagagaacgtagatagcgacgccgccgccgcctcaaaccggcatccaccgcgggagaaggagggtacgcacactggggcttcggggagtagccgacttcgtcccgatccagccgctgttgcgcgagccgattataccggacgacgtggacgcactcccatctgaccttcagtaggtacaggtgtttcgccaccatacgcgggcacaactaccacattcacgtcaaccggtattgcctggtcaattgccagaccactgggccgacgctctcgagccacatggactctgcgctcattcgggagctcagtttatgcatcagcaacgttcaggcgtggtccatgaccatagggtggtttttcacgccgccgccgaccgccatctccccggtcgcatcccaccggccccgaggcccccgctctagaaggttgtattccgctactataggcatctacatggacctgtaggtccactacggccgccgccaccacctccgcgcgcctttcagcgcctgcgccaaggtctacaacgcgtcgccgtttttcacgaggtaccagccctgcgagaccggccagtccgcgcgcgttagcaactgcgagatctggcacgttttcctctcggacattcgcccgtgagaaggcaccagaccacctatttaagcgttcccatagtaccgcctgctggccccaagctcggggcataggccggcaggcggcactaggtacgccaatggcgggcgcacagcttgggtccacacgctgcagtctgttgccccctcacgaggaaaaccgaaggaaggtccgcgccgccgacgacgcgatcgaaaaaaccggtgaccggcgcgcgtcgcattcgccaatccgacctttcgctttcgtaattcaccgagcgagggacatcggcctcccaataaaaggttcccaactcagcgccctgggggccaagctcagagcctggccggcctgacgccgcttgcccccaaacggaggggcagtacgttctggggcgaacgtttaaggaggcctttgtccctgctcggggaaaaaacgaaaagggtctacgtaggccacgacgccgtctacgcggggggaggagtcgtcgccgttctcgttctcgtcgccgtctgtacgtcccgtgggaggggaggaggatggcgcagtcctccccgctgtaggcgccaactgcgccgtcgtctaccactaatgcttgggggcgccgcggcccgggccgtgatggacctgaacctcctcccgctcccggaccgcgccgatcctcgcgggagaggactcgccgtgggttcccacgtcgacttcgcactatgcgcactccgcatgcacggcgccgtcttggacaaagcgctggcgctccctctcctcgggctcctctacgccctagctttcaaggtgcgtcccgcgctcgacgccgtaccggacttagcgctcgccaacgacgcgctcctcctgaaactcgggctgcgcgcttggccctaatcagggcgcgcgcgtgtgcaccgccggcggctggaccattggcgtatgctcgtctgccacttggtcctctaattgaaagttttttcgaaattgttggtgcacgcatgcgaacaccgcgcgctcctccaccgatatcctgactacgtagacaccctgaaacattcgcgcgacctcgttttgggtttatcgttcggcgagtaccgcgtcgacaaggaatatcacgtcgtgtcgtccctgttgctccgtaagtccctacgcgacgatttgtatcatctcgggctcccggcgaccgacgagctaaactatttgtaggacgtctcgtatcaccacgtcctcgcgtcgaactcggaccgactgttccaccggcggtagttgataaggtacgaatcggacccgttcaaaatgcgggcgttctatatggtatggggaatgcaagggtatctgttcctccatttctagctccccaagatgtacgcgtaccgcgacttccacgaatggaactcgctgctggacccgcaaatagcgttgctcgcgtaggtgttccggcactcgcactcggccgccgcgctcgagtcgctggcgctcgactacgtgtcggacgtttcccgggaccgaccgtgcccgtcgccgctatctctccggctcaggatgaaactgcgcccgcgactggacgcgacccggggttcggctgcgcgggacctccgtcgaccccggcctggacccgaccgccaccgtgggcgcgcgcgaccgttgcagccgccgcacctccttatactgctcctgctactcatgctcggtctcctgccgctcatgattcgccactacaaagactagtctactacgttctgcgttgcctgggccgccacgcccgccgcgacgtctcggtcggcaggccggaattgaggtgcctgctgaccgcggtccagtacctggcgtagtacagcgactgacgcgcgttaggactgcgcaaggccgtcgtcggcgtccggttggccgagaggcgttaagaccttcgccaccagggccgcgcgcgtttggggtgcgtgctcttccacgaccgctagcatttgcgcgaccggcttttgtcccggtaggccgggctgctccggccggaccagatgctgcgcgacgaagtcgcgcaccgagcaatgttgtcgccgttgcacgtctggttggacctggccgaccaccccctacacgcgctccggcaccgcgtcgcactcgcgcgcgtcgtcgtcccgttggacccgaggtaccaacgtgatttgcggaaggactcatgtgtcgggcggttgcacggcgcccctgtcctcctgatgtggttgaaacactcgcgtgacgccgattaccactgactctgtggcgtttcactccacatggtcagacccggtctgataaaaaaggtctggtcatctgttccggacgtctggcatttggactcggtccgaaagtttttgaacgtccccgacaccccccacgcccgagggtgtccgctggcgcgctggcacagatcgaacgactgcgggttgagcgcggacaacgacgacgattatcgcgggaagtgcctgtcaccgtcgcacagggccctgtgtatggatccagtgaacgactgtgacatggcgctccggtatccagtccgcgtacacctgctcgtatgaaaggtcctctaatgttcacagtcggcgcgcgaccccgtcctcctgtgcccgtcggacctccgttgggatttgatggacgactggttggccgccgtcttctaggggagcaacgtgtcaaatttgtcgctcctcctcgcgtaaaacgcgatgcacgtcgtctcgcactcggaattggactacgcgctgccccattgcgggtcgcaccgcgacctgtactggcgcgcgttgtaccttggcccgtacatacggagtttggccggcaaatagttggcggattacctgatgaacgtagcgcgccggcggcacttggggctcataaagtggttacggtagaacttgggcgtgaccgatggcgggggaccaaagatgtggccccctaagctccacgggctcccattgctacctaaggagaccctgctgtatctgctgtcgcacaaaaggggcgttggcgtctgggacgatctcaacgttgtcgcgctcgtccgtctccgccgcgacgctttcctttcgaaggcgtccggttcgtcgaacaggctagatccgcgacgccggggcgccagtctacgatcatcgggtaaaggttcgaactatcccagagaatggtcgtgagcgtggtgggcgggcgcggacgacccgctcctcctcatggatttgttgagcgacgacgtcggcgtcgcgctttttttggacggaggccgtaaagggttgttgccctatctctcggatcacctgttctactcatctaccttctgcatgcgcgtcctcgtgtccctgcacggtccgggcgcgggcgggtgggcagcagtttccgtgctggcagtcgccccagaccacaccctcctgctactgagccgtctgctgtcgtcgcaggacctaaaccctccctcaccgttgggcaaacgcgtggaagcggggtccgacccctcttacaaaatttttttttttttcgtactacgttttattttttgagtggttccggtaccgtggctcgcaaccaaaagaacataaggggaatcatacgccgcgcgccgctacatactccttccaggaggagggaggatgctctcacaccactcgcgccgcggtcaccgccgccgcgacccaagagggaagctacgaggggacctgggcggcaaacacggaggcgccatggacgccggatggcccccctctttgtcgtaggcaatgagactcaaccgtggggataagctgtggtgggcacacatggaccacctgttgttcagttgcctacaccgtagggacttgatggtcttgctggtgtcgttgaaagactggtgccagtaagttttgttactgatgtcgggccccctccgttcgtgtgtctggtagttagaactgctggccagcgtgaccccgccgctggacttttggtaggacgtatggttgtacggtttacacttgctcaagtacaaatggttattcaaattccgcgcccactaccacagcgcgaacggatgattcctgttagtccacctcgactttatgctcacccacctcaagtgcgacgggctcccgttgatgaggctctggtactggtatctggaatacttgttgcgctagcacctcgtgatgaactttcacccgtctgtcttgccccaagacctttcgctgtagccccatttcaaactgtgggcgttgaagtctgaccccaaactggggcagtgaccagaacagtacggaccccatatatgtttgcttcggaaggtaggtctgtagtaaaacgacggtcctacgccccacctgaagtgggtgtcggcggactcgttgaacaacccgtaggcgttcgccgttgggaaggtcctcccgaaatcctagtggatgctactagacctcccaccattgtaagggcgtgacaacctacacctgcggatggtccgctcgaactttctactgtggcttgtcccgcccccaccgcgtccgccgtcgttgtcgtcaccgtcgccgcgccttctcttgaggttgcgccgtcggcgccgttacgtcggccacctcctgtacttgctagtacggtaagcgccgctgtggaaacggtgtgcccgactcctcttcgcgcgactccggcttcgtcgccggcttcgacggcgggggcgacgcgttgggctccagctcttcggagtcttctttggccactagtttggggactgtctcctgtcgttctttgcgtcaatgttggattattcgttactgtcgtggaagtgggtcatggcgtcgaccatggaacgtatgttgatgccgctgggagtctggccttaggcgagtacctgggacgaaacgtgaggactgcattggacgccgagcctcgtccagatgaccagcaacggtctgtactacgttctggggcactggaaggcgaggtgcgcggtctagtcgttgaaaggccaccacccgcggctcgacaacgggcacgtgaggttctcgaagatgttgctggtccggcagatgagggttgagtaggcggtcaaatggagagactgggtgcacaagttagcgaaagggctcttggtctaaaaccgcgcgggcggtcgggggtggtagtggtggcagtcacttttgcaaggacgagagtgtctagtgccctgcgatggcgacgcgttgtcgtagcctcctcaggtcgctcactggtaatgactgcggtctgcggcgtggacggggatgcaaatgttccgggacccgtatcagagcggcgcgcaggatagctcggcgtgaaaaactcgttcgtacaggtaggaatatagcgggtcgttattgtgtccgaccccggacgcgaagggttcgttctacaaaccgccccggttcttcgcgaggctggttgtgggtcacgcgcacgcgcccgtgatggcgcgcgggaccccgcgcgtgtttgcgccggcgtgacccgcgtggtggcagctactgcggtagctgcgccaccacctcctccgcgcgttgatgtgcgggtgcggcggtggtcacaggtgtcacctgcgccggtaagtctggcaccacgcgcctcgggccgcgatacgattttacttctctgccgcctccgcgcatcgtgcagcggtggcggcggctgggccgtgacggcgggttgcgcgccgccgccgggacgaattggcgcgtgcagcgtggccggctgcccgccggtacgcccggcgagcttccgaccggcgcccataacagtgacacggggggtccaggtccgctgctcgccggcggcgtcgtcggcgccggtaatcacgatactgagtcccagcgtccccgttgcacataacccacgcgctgagccaatcgccggacgcgcacgggcacgcgtgggcggggggcgcgttgatctaacgttcttttttgatgaatctgagcatgacaacatacataggtcgccgccgccgcgcgttgcttcgatacaggttcgcgttttagtttcttctctacgaggtccagtagcgcggcctctagataccggggggcttcttccttctcgtcctaatgttcggggctttcgatttcgcccagtttttctttttctttctactactactacttgaactgctgctccaccttgacgacgtgcgatggcgcgggtccgctgcccatgtcacctttccagctgcgcattttgcacaaaacgctgggccgtggtggcatcagaaatgcgggccactcgcgaggtgggcgtggatgttcgcgcacatactactccacatgccgctgctcctggacgaactcgtccggttgctcgcggagcccctcaaacggatgcctttcgccgtattcctgtacgaccgcaacggcgacctgctcccgttgggttgtggatcggatttcgggcattgtgacgtcgtccacgacgggcgcgaacgtggcaggcttcttttcgcgccggatttcgcgctcagaccactgaaccgtgggtggcacgtcgactaccatgggttcgcggtcgctgaccttctacagaaccttttttactggcaccttggacccgacctcgggctccaggcgcacgccggttagttcgtccaccgcggccctgacccgcacgtctggcacctgcaagtctatgggtgatggtcatcgtggtcataacggtggcggtgtctcccgtacctctgtgtttgcaggggccaacggagtcgccaccgcctacggcgccacgtccgccagcgacgccggcgcaggttctggagatgcctccacgtttgcctgggcacctacaaagcgcaaagtcggggggccgcgggcgcggcaagctccttcatgccgcggcggtcgcgcgatgacgggcttatacgggatgtaggaaggtaacgcggatgggggccgatagcaccgatgtggatggcggggtcttctgctcgttgatgggctgcggcttggtggtgaccttgggcggcggcggcagcggcagcggtcgggcacgaccggggctaaaggcacgcgtcccaccgagcgcttcctccgtcctgggaccacgacggttgtcgcgcgatggtggggtcgtagcaaattttcggccagaaacaccaagaacgtctataccgggagtggacggcggaggcaaagggccacggccctaaggctccttcttacgtggcatcctccccgtaccggccggtgccggactgcccgccgtacgcagcacgcgtggtggccgccgccgcgcgcagcgtggcagcgtacgcgccgccataggacggggaggaataaggtgactagcggcgccgctaaccgcggcacgggccttaacgtaggcaccggaacgtccgcgtctctgtgactaatttttgttcaacgtacacctttttagttttatttttcagacctgagagtgcgagcgaaccaggacattgataaaacatcttaccttctgtagttgaaacgcagagaccggggcgctgtgccgagcgcgggcaagtaccctttgaccgttctatagccgtggtcgttatactcgccaccgcggaagtcgaccccgagcgacacctcgccgtaatttttaaagccaaggtggcaattcttgataccgtcgttccggaccttgtcgtcgtgtccggtctacgactccctattcaactttctcgttttaaaggttgttttccaccatctaccggaccggagaccgtaatcgccccaccacctggaccggttggtccgtcacgttttattctaattgtcattcgaactaggggcgggagggcatctcctcggaggtggccggcacctctgtcacagaggtctccccgcaccgcttttcgcaggcgcggggctgtcccttctttgagaccactgcgtttatctgctcggagggagcatgctcctccgtgatttcgttccggacgggtggtgggcagggtagcgcgggtaccgatggcctcacgacccggtcgtgtgtgggcattgcgacctggacggaggggggcggctgtgggtcgtctttggacacgacggtccgggctggcggcaacaacattgggcaggatcggcgcgcagggacgcggcgcggcggtcgccaggcgctagcaacgccgggcatcggtcaccgttgaccgtttcgtgtgacttgtcgtagcacccagacccccacgttagggacttcgcggctgctacgaagactatcgattgcacagcatacacacagtacatacgcaggtacagcggcggtctcctcgacgactcggcggcgcgcgggcgaaaggttctaccgatggggaagctactacggcgtcaccagaatgtacgtgtagagcccggtcctgcggagcctcatggactcggggcccgaccacgtcaaacgggcgcggtggctctgcatgaagtcggacttattgttcaaatctttggggtgccaccgcggatgcgtgctgcactggtgtctggccagggtcgcaaactgcgacgccaagtagggacacctggcactcctatgacgcatgagcatgttccgcgccaagtgggatcgacacccactattggcacacgacctgtaccgaaggtgcatgaaactgtaggcgccgcacgacctgtccccgggatgaaaattcgggatgagaccgtgacggatgttgcgggaccgagggttcccacggggtttaggaacgcttaccctacttcgacgatgacgagaactttatttggatcttcttctcctgctactgttgcttctgcttcatctgctcgttcgactcgtcgttttttgagtgcataaacccgtccgcggaataagaccatatttataatgtttcctcccataagtttatccacagcttccagtttgtggatttatacggctattttgtaaagttggacttggagtttatcctcttagagtcaccatgctttgtctttaattagtacgtcgaccctctcaggattttttctgatggggttactttggtacaatgccaagtatacgttttgggtgtttacttttacctcccgttccgtaagaacatttcgttgttttacctttcgatctttcagttcacctttacgttaaaaagagttgatgactccgtcggcgtccgttaccactattgaactgaggatttcaccataacatgtcacttctacatctatatctttggggtctgtgagtataaagaatgtacgggtgataattccttccattgagtgctcttgattacccggttgttagatacgggttgtccggattaatgtaacgaaaatccctgttaaaataaccagattacataatgttgtcgtgcccattatacccacaagaccgcccggttcgtagcgtcaacttacgacaacatctaaacgttctgtctttgtgtctcgaaagtatggtcgaaaacgaactaaggtaaccactatcttggtccatgaaaagatacaccttagtccgacaactgtcgatactaggtctacaatcttaataacttttagtaccttgacttctacttgaaggtttaatgacgaaaggtgaccctccacactaattatgtctctgagaatggttccattttggattttgtccagtccttttacctaccctttttctacgatgtcttaaaagtctatttttactttattctcaacctttattaaaacggtacctttagttagatttacggttggacacctctttaaaggacatgaggttgtatcgcgacataaacgggctgttcgatttcatgtcaggaaggttgcatttttaaagactattgggtttgtggatgctgatgtacttgttcgctcaccaccgagggcccgatcacctgacgatgtaattggaacctcgtgcgaccagggaactgatatacctgttgcagttgggtaaattggtggtggcgttacgaccggacgcgatggcgagttacaacgacccgttaccagcgatacacgggaaggtgtaggtccacggagtcttcaagaaacggtaatttttggaggaagaggacggcccgagtatgtggatgctcaccttgaagtccttcctacaattgtaccaagacgtctcgagggatcctttactggattcccaactgcctcggtcgtaattcaaactatcgtaaacggaaatgcggtggaagaaggggtaccgggtgttgtggcggaggtgcgaactccggtacgaatctttgctgtggttgctggtcaggaaattgctgatagagaggcggcggttgtacgagatgggatatgggcggttgcgatggttgcacgggtataggtaggggagggcgttgacccgccgaaaggcgccgacccggaagtgcgcggaattctgattcctttggggtagtgacccgagcccgatgctgggaataatgtggatgagaccgagatatgggatggatctaccttggaaaatggagttggtgtggaaattcttccaccggtaatggaaactgagaagacagtcgaccggaccgttactggcggacgaatgggggttgctcaaactttaattcgcgagtcaactgcccctcccaatgttgcaacgggtcacattgtactggtttctgaccaaggaccatgtttacgatcgattgatattgtaaccgatggtcccgaagatatagggtctctcgatgttcctggcgtacatgaggaagaaatctttgaaggtcgggtactcggcagtccaccacctactatgatttatgttcctgatggttgtccacccgtaggatgtggttgtgttgttgagacctaaacaaccgatggaacgggggtggtacgcgcttcctgtccggatgggacgattgaaggggataggcgaatatccgttctggcgtcaactgtcgtaatgggtctttttcaaagaaacgctagcgtgggaaaccgcgtagggtaagaggtcattgaaatacaggtacccgcgtgagtgtctggacccggttttggaagagatgcggttgaggcgggtgcgcgatctgtactgaaaactccacctagggtacctgctcgggtgggaagaaatacaaaacaaacttcagaaactgcaccaggcacacgtggtcggcgtggcgccgcagtagctttggcacatggacgcgtgcgggaagagccggccgttgcggtgttgtatttcttcgttcgttgtagttgttgtcgacggcggtacccgaggtcactcgtccttgactttcggtaacagtttctagaaccaacacccggtataaaaaacccgtggatactgttcgcgaaaggtccgaaacaaagaggtgtgttcgagcggacgcggtatcagttatgccggccagcgctctgacccccgcatgtgacctaccggaaacggaccttgggcgtgagtttttgtacgatggagaaactcgggaaaccgaaaagactggtcgctgagttcgtccaaatggtcaaactcatgctcagtgaggacgcggcatcgcggtaacgaagaagggggctggcgacatattgcgaccttttcaggtgggtttcgcatgtccccgggttgagccggcggacacctgataagacgacgtacaaagaggtgcggaaacggttgaccggggtttgagggtacctagtgttggggtggtacttggaataatggccccatgggttgaggtacgagttgtcaggggtccatgtcgggtgggacgcagcgttggtccttgtcgagatgtcgaaggacctcgcggtgagcgggatgaaggcgtcggtgtcacgcgtctaatcctcgcggtgaagaaaaacagtgaactttttgtacatttttattacatgatctctgtgaaagttatttccgtttacgaaaataaacatgtgagagcccactaataaatgggggtgggaacggcagacgcggcaaatttttagtttccccaagacggcgcgtagcgatacgcggtgaccgtccctgtgcaacgctatgaccacaaatcacgaggtgaatttgagtccgtgttggtaggcgccgtcgagccacttcaaaagtgaggtgtccgacgcgtggtagtggttgcgcaaatcgtccagcccgcggctatagaacttcagcgtcaaccccggaggcgggacgcgcgcgctcaacgctatgtgtcccaacgtcgtgaccttgtgatagtcgcggcccaccacgtgcgaccggtcgtgcgagaacagcctctagtctaggcgcaggtccaggaggcgcaacgagtcccgcttgcctcagttgaaaccatcgacggaagggtttttcccgcgcacgggtccgaaactcaacgtgagcgtggcatcaccgtagttttccactggcacgggccagacccgcaatcctatgtcgcggacgtattttcggaactagacgaattttcggtggactcggaaacgcggaagtctcttcttgtacggcgttctgaacggccttttgactaaccggcctgtccggcgcagcacgtgcgtcgtggaacgcagccacaacctctagacgtggtgtaaagccggggtggccaagaagtgctagaaccggaacgatctgacgaggaagtcgcgcgcgacgggcaaaagcgagcagtgtaggtaaagttagtgcacgaggaataaatagtattacgaaggcacatctgtgaattcgagcggaagctagagtcgcgtcgccacgtcggtgttgcgcgtcgggcacccgagcactacgaacatccagtggagacgtttgctgacgtccatgcggacgtccttagcggggtagtagcagtgtttccagaacaacgaccacttccagtcgacgttgggcgccacgaggagcaagtcggtccagaacgtatgccggcggtctcgaaggtgaaccagtccgtcatcaaacttcaagcggaaatctagcaataggtgcaccatgaacaggtagtcgcgcgcgcgtcggaggtacgggaagagggtgcgtctgtgctagccgtgtgagtcgcccaagtagtggcattaaagtgaaaggcgaagcgacccgagaaggagaaggagaacgcaggcgtatggtgcgcggtgacccagcagaagtaagtcggcggcgtgacacgcgaatggaggaaacggtacgaactaatcgtggccacccaacgactttgggtggtaaacatcgcggtgtagaagagaaagaaggagcgacaggtgctaatggagaccactaccgcccgcgagcccgaaccctcttcccgcgaagaaaaagaagaacccgcgttaccggtttaggcggcggctccagctaccggcgcccgacccacacgcgccgtggtcgcgcagaacactactcagaaggagcaggagcctgagctatgcggcggagtaggcgaaaaaacccccgcgggcccctccgccgccgctgcccctgcccctgctgtgcaggaggtaccaaccccctgcagcgcggcgtggcgcaggcgcgagcccccaccaaagcgcgacgaggagaagggctgaccggtaaaggaagaggatatccgtctttttctagtacctcagtcagctcttcttcctgtcggattggcgggggagactcaagcggtggtggcggaggtggctacggcggttgcgcggatggtggaaggggcagctccgtgggggcgaactcctcctccttcactaatagctcgtcctgggtccaaaacattcgcttctgctgctcctggcgagtcatggttgtctcctatttttcgttctggtcctgttgcgtctccgtttgctccttgttcagcccgcccccctgctttccgtaccgctgatggatctacaccctctgctgcacgacaacttcgtagacgtcgcggtcacgcggtaatagacgctgcgcaacgttctcgcgtcgctacacggggagcggtatcgcctacagtcggaacggatgcttgcggtggataagagtggcgcgcatggggggtttgcggttcttttgccgtgtacgctcgggttgggcgcggagttgaagatggggcataaacggcacggtctccacgaacggtggatagtgtagaaaaaggttttgacgttctatggggataggacggcacggttggcgtcggctcgcctgttcgtcgaccggaacgccgtcccgcgacagtatggactatagcggagcgagttgcttcacggtttttagaaactcccagaacctgcgctgctcttcgcgcgccgtttgcgagacgttgtccttttgtcgcttttactttcagtgagacctcacaaccaccttgagctcccactgttgcgcgcggatcggcatgattttgcgtcgtagctccagtgggtgaaacggatgggccgtgaattggatggggggttccagtactcgtgtcagtactcactcgactagcacgcggcacgcgtcggggacctctccctacgtttaaacgttcttgtttgtctcctcccggatgggcgtcaaccgctgctcgtcgatcgcgcgaccgaagtttgcgcgctcggacggctgaacctcctcgctgcgtttgattactaccggcgtcacgagcaatggcacctcgaactcacgtacgtcgccaagaaacgactgggcctctacgtcgcgttcgatctcctttgtaacgtgatgtggaaagctgtcccgatgcatgcggtccggacgttctagaggttgcacctcgagacgttggaccagaggatggaaccttaaaacgtgcttttggcggaacccgttttgcacgaagtaaggtgcgagttcccgctccgcgcggcgctgatgcaggcgctgacgcaaatgaataaagatacgatgtggaccgtctgccggtacccgcaaaccgtcgtcacgaacctcctcacgttggagttcctcgacgtctttgacgatttcgttttgaacttcctggatacctgccggaagttgctcgcgaggcaccggcgcgtggaccgcctgtagtaaaaggggcttgcggacgaattttgggacgttgtcccagacggtctgaagtggtcagtttcgtacaacgtcttgaaatccttgaaataggatctcgcgagtccttagaacgggcggtggacgacacgtgaaggatcgctgaaacacgggtaattcatggcgcttacgggaggcggcgaaaccccggtgacgatggaagacgtcgatcggttgatggaacggatggtgagactgtattaccttctgcactcgccactgccagatgacctcacagtgacagcgacgttggatacgtggggcgtggcgagggaccaaacgttaagcgtcgacgaattgctttcagtttaatagccatggaaactcgacgtcccagggagcggactgcttttcaggcgccgaggccccaactttgagtgaggccccgacacctgcagccgaatggaagcgtttaaacatggactcctgatggtgcgggtgctctaatccaagatgcttctggttagggcgggcggattacgcctcgaatggcggacgcagtaatgggtcccggtgtaagaaccggttaacgttcggtagttgtttcgggcggttctcaaagacgatgctttccctgccccccaaatgaacctgggggtcaggccgctcctcgagttgggttaggggggcggcggcgtcgggatagtcgtcgtcggcgcccgggaacgaagggtcctaccgtgggtttttcttcgacgtcgacggcggcggtgggtgcctgctcctccttatgaccctgtcagtccgtctcctccaaaacctgctcctcctcctcctgtactaccttctgaccctctcggatctgctccttcgaaggctccagcttctccacagtctgctttgtggcagtgggagccagcgtaaggggagcggccgcggggtctttagccgttggccaaggtcgtaccgatgttggaggcgaggagtccgcggcggccgtgacgggcaagcggctgggttggcatctaccctgtggtgaccttggtcccggccattcaggttcgtcggcggcggcaatcgggttctcgttgttgtcgcggttccgatggcgagtaccgcgcccgtgttcttgcggtatcaacgaacgaacgttctgacacccccgttgtagaggaagcgggcggcgaaagaagagatggtagtgccgcaccggaagggggcattgtaggacgtaatgatggcagtagagatgtcgggtatgacgtggccgccgtcgccgtcgttgtcgtcgccggtgtgtcttcgtttccgctggcctatcgttctgagactgtttcgggttctttaggtgtcgccgccgtcgtcgtcctcctcctcgcgacgcagaccgcgggttgcttgggcatagctgggcgctcgaatctttgtcctaaaaagggtgagacatacgatataaagttgtctcgtccccggttcttgttctcgacttttattttttgtccagagacgctagggagtgggcgtcgacggacatagtgttttcgcttctagtcgaagccgcgtgcgaccttctgcgcctccgagagaagtcatttatgacgcgcgactgagaattcctgatcaaagcgcgggaaagagtttaaattcgcgcttttgatgcagtagaggtcgccggtgtgggccgcggtcgtggacaacagtcgcggtaatactcgttcctttaagggtgcgggatgtacacctcaatggtcggtgtttaccctgaacgccgacctcgacgggttctgatgagttgggcttatttgatgtactcgcgccctggggtgtactatagggcccagttgccttatgcgcgggtggctttggcttaagaggaccttgtccgccgataatggtggtgtggagcattattggaattaggggcatcaaccgggcgacgggaccacatggtcctttcagggcgagggtggtgacaccatgaagggtctctgcgggtccggcttcaagtctactgattgagtccccgcgtcgaacgcccgccgaaagcagtgtcccacgccagcgggcccgtcccatattgagtggactgttagtctcccgctccataagtcgagttgctgctcagccactcgaggagcgaaccagaggcaggcctgccctgtaaagtctagccgccgcggccggcgagaagtaagtgcggagcagtccgttaggattgagacgtctggagcaggagactcggcgcgagacctccgtaaccttgagacgttaaataactcctcaaacacggtagccagatgaaattggggaagagccctggagggccggtgataggcctagttaaataaggattgaaactgcgccatttcctgagccgcctgccgatgctgacttacaattcacctctccgtctcgttgacgcggactttgtggaccaggtgacagcggcggtgttcacgaaacgggcgctgaggccactcaaaacgatgaaacttaacgggctcctagtatagctcccgggccgcgtgccgcaggccgaatggcgggtccctctcgaacgggcatcggactaagccctcaaatgggtcgcgggggacgatcaactcgccctgtcccctgggacacaagagtgacactaaacgttgacaggattgggacctaatgtagttctagaaacaacggtagagacacgactcatattatttatgtctttaattttatatgaccccgaggatagcggtaggacatttgcggtggcagaagtgggcgggttcgtttggttccgcttggaatggaccatgaaaattgtagagagggagacactaaatgttgtcaaagttgggtctgcctcactcagatgctctcttggagaggctcgagtcgatgaggtagtcttttttgtggtgggaggaatggacggcccttgcatgctcacgcagtggccggcgacgtggtgtggatggcggactggcatttggtctgaaaaaggcctgtctggagttattgagacaaatggtcttgtcctccactcgaatcttttgggaatcccataatccggtttccgcgtcgatgacaccccaaatacttgttaagttcgttgagatgcccgataagattaagtccaaagagatcttagccccaaccccaataagagacagaacactaagagaaataagaatatgattgcgaagagacggattccgagcggcggacgacacacgtgtaaacgtaaataacagtcgaaaaatttgcgaccccagcggtgggttctactaatccatgtattaggatccaaatgagtgggaacgcagtcgggtgccatggtgggttttccacctaaaattcctcggtcggacattacaatgtaagcgtcgacttcgattactcacgtggtgagaatattttacgtggtgtcttgtacttttcgacgaataagcggtgtttttgttttaaccgttcatacgacaaatacgataaaccgtcggtccactgtgatgtctcatattacaatgtcaaaaggtcccattttcagtattttgaaaatacatatgaaaaggtaaaatactttacacgctgtaatggtacatgtactcgtttgtcatattcaacaccgggggtgttttaacacaccttttgtgaccgtgaaagacgacgtgacgatacgattaatgtcacgagcgaaaccagacatgggatgagatataatttatgttttcgtctgcgtcgaaataactccttttcttttacggaattaaatgattcaatgtttcgattacagtggtgattgacgaaatgagcgacgaacgttttgtttaagtttttcaatcgtaatattaatcttatcctaaatttggggggccagtaaaggacgagttatggtaaggggacttgttaactgagatacaccctatacgaggtcgcgatgttggaacttcagtccgaaggacctacagtcgtagactgaaaccggtcgtggacagggcgcctaaacaaggtcaggttgatgtcgctgggtgggattgtctctactggttgtgttggttgcgccggcggcgatggcctgaatgtagatggtgtttatgtggggttcaaagacggaaacagttattgaccctattgaacccgtacaccaccaagaggtatcgcgaatacaaacatacggaataataatacaccgagtagacgacggatttcgcgtttgcgcgggctggtgggtagatatcagggtagtaacacgatgtgggtttgttactaccttaggtatctaacctgcctgactttgtgtacaagaaaagagaatgtcatactaatttactctgtactaaggagctcaaaaatataatgactgggaacaacgcgaaaaaacacgcacgaggtgtaaccgacgccaaagagtgtagcttcatctgacgtaaggtcggaagtgtcagataaacgaaatgcctaaacagtgggagtgcgagtagacgtcggagtagtgacaccagtagcggaaataggtcacgtaactgacccagacacacgcgaaacgtatagagtctgtggtaggggtcatgtccctgtcctgatatcgactcgaagaatcttaagaaattaatactttaaatgacactgaaaagacgactaataaacgtgggatagacgcaaaacaaggggctggaggttcggagtttctgtatatagtacgtctaagtgagcatataccttataaggttcaacgatgttacttttttcgctagaaaggcttcggaccaatatacgttagtagagacaataccacaagacgtcatggtagaatcgggatcgatatatagggatggaactgtaaccgaccttgcgttatctacggtacttggtgggttgaaaggggcgcgggcgatacgaaggtgacgttgttcaacaacggccgccgaaacagggtcggttagtcggagcgggtggaagagggtgggggtgactttagtcgatgaaattagattgtcctcctctactgactgtgggatctagatctttacctgccttaataatgtctcgtcgcggacgatctttctgcgtcccgtcgccggctcgttgtcgcgtacttagttctcgaggttctgtaccaattgaacgtggtcacgttttccccatagaaaacagagcatttcgtccggtttcagtggatgctgtcattatggtggcctgtggcggaatcgatgttcaacggttggttcgcagtctttaaccaccagtaccaccctcttttcgggtaatggtattgagtcgtgagccatctttggcttccgacgtaagtgagtggaacagttcctggactcctagagacgtgggaataattctgggacacgccagagtttctagaataagggaaattgattatttttttttattatttcgtagtgaatgaattttagtcaatcgtttaaagacaggtcaaataagtcgtcgtggaggaacgggaggagggtcgagaccataacgtcgaaggaggaccgacgtttgaaagaggtgttagatttaccttacagtcaaaggaggacaaggacaggtaggcgtgggtgatagaagtacaacaacgtctacttcgcgcgttctggcagacttctatggaagttggggcacataggtatactgtgcctttggccaggaggttgacacggaaaagaatgaggagggaaacatagggggttacccaaagttctctcagggggaccccatgagagaaacgcggataggcttggagatcaatggaggttaccgtacgaacgcgagttttacccgttgccggagagagacctgctccggccgttggaatggagggttttacattggtgacactcgggtggagagtttttttggttcagtttgtatttggacctttatagacgtggggagtgtcaatggagtcttcgggattgacaccgacggcggcgtggagattaccagcgcccgttgtgtgagtggtacgttagtgtccggggcgattggcacgtgctgaggtttgaatcgtaacggtgggttcctggggagtgtcacagtcttcctttcgatcgggacgtttgtagtccgggggagtggtggtggctatcgtcatgggaatgatagtgacggagtgggggagattgatgacggtgaccatcgaacccgtaactgaactttctcgggtaaatatgtgttttaccttttgatcctgatttcatgccccgaggaaacgtacattgtctgctggatttgtgaaactggcatcgttgaccaggtccacactgataattattatgaaggaacgtttgatttcaatgacctcggaacccaaaactaagtgttccgttatacgttgaattacatcgtcctcctgattcctaactaagagttttgtctgcggaatatgaactacaatcaataggcaaactacgagttttggttgatttagattctgatcctgtcccgggagaaaaatatttgagtcgggtgttgaacctataattgatgttgtttccggaaatgaacaaatgtcgaagtttgttaaggtttttcgaactccaattggattcgtgacggttccccaactacaaactgcgatgtcggtatcggtaattacgtcctctacccgaacttaaaccaagtggattacgtggtttgtgtttaggggagttttgtttttaaccggtaccggatcttaaactaagtttgttccgataccaaggatttgatccttgaccggaatcaaaactgtcgtgtccacggtaatgtcatcctttgtttttattactattcgattgaaacacctggtgtggtcgaggtagaggattgacatctgatttacgtctctttctacgatttgagtgaaaccagaattgttttacaccgtcagtttatgaacgatgtcaaagtcaaaaccgacaatttccgtcaaaccgaggttatagaccttgtcaagtttcacgagtagaataatattctaaactgcttttacctcacgatgatttgttaaggaaggacctgggtcttataaccttgaaatctttacctctagaatgacttccgtgtcggatatgtttgcgacaacctaaatacggattggatagtcgaataggttttagagtgccattttgacggttttcattgtaacagtcagttcaaatgaatttgcctctgttttgatttggacattgtgattggtaatgtgatttgccatgtgtcctttgtcctctgtgttgaggttcacgtatgagatacagtaaaagtaccctgaccagaccggtgttgatgtaattactttataaacggtgtaggagaatgtgaaaaagtatgtaacgggttcttatttcttagcaaacacaatacaaagttgcacaaataaaaagttaacgtcttttaaagttcagtaaaaagtaagtcatcatatcggggtggtggtgtatcgaatatgtctagtggcatggaattagtttgagtgtcttgggatcataagttggacggtggagggagggttgtgtgtctcatgtgtcaggaaagaggggccgaccggaatttttcgtagtatagtacccattgtctgtataagaatccacaatataaggtgtgccaaaggacagctcggtttgcgagtagtcactataattatttgaggggcccgtcgagtgaattcaagtacagcgacaggtcgacgactcggtgtccgacgacaggttgaacgccaacgaattgcccgccgcttcctcttcaggtgcggatgtacccccatctcagtattagcacgtagtcctatcccgccaccacgacgtcgtcgcgcgcttatttgacgacggcggcggcgaggcaggacgtccttatgttgtaccgtcaccagaggagtcgctactaagcgtggcgggcgtcgtattccgcggaacaggaggcccgtgtcgtcgcgtgggactagagtgaatttagtcgtgtcattgacgtcgtgtcgtggtgttataacaagttttagggtgtcacgttccgcgacataggtttcgagtaccgcccctggtgtcttgggtgcaccggtagtatggtgttcgcgtccatctaattcaccgctggggagtatttgtgcgacctgtatttgtaatggagaaaaccgtacaacattaagtggtggagggccatggtatatttggagactaatttgtaccgcggtaggtggtggtaggatttggtcgaccggttttggacgggcggccgatatgtgacgtcccttggccctgaccttgttactgtcacctctcgggtcctgagcattggtacctagtagtacgagcagtactatagttacaaccgtgttgtgtccgtgtgcacgtatgtgaaggagtcctaatgttcgaggagggcgcaatcttggtatagggtcccttgttgggtaaggacttagtcgcatttagggtgtgacgtcccttctggagcgtgcattgagtgcaacacgtaacagtttcacaatgtaagcccgtcgtcgcctactaggaggtcataccatcgcgcccaaagacagagttttcctccatctgctagggatgacatgcctcacgcggctctgttggctctagcacaaccagcatcacagtacggtttaccttgcggcctgcatcagtataaaggacttcgttttggtccacgcccgcactgtttgtctagacgcagaggccagagcggcgaatctagcgagacacatcatcaacatcatataggtgagagagtttcgtaggtccgcgggggaccgaagcccaagatacatttgaggaagtacgcggcgacgggactattgtaggtggtggcgtcttattcggtgtgggtcggttggatgtgtaagcaagacgctcagtgtgtgccctcctcgcccttctcgaccttcttggtacaaaaaaaaaaataaggttttctaataggttttggagttttacttctagataattcacttgcgcgaggggaggccaccgcaccagtttgagatgtcggtttcttgtctattaccgtaaacattctacaacgtgttaccgaaggttttccgtttgccgggagtgcaggttcacctgcatttccgatttgggaagtcccacttagaggagatatttgtaaggtcgtggaagttggtacgggtttattaagagtagagcggtggaagagttatatagagattcgtttagggcttataattcaggccggtaacatttttagacgaggtctcgcgggaggtggaagtcggagttcgtcgcttagtactaacgtttttaagtccaaggagtgtctggacatattctaagttttcgccttgtaattgtttttatggcgctagggcatccagggaagcgtcccggtcgacttgtattagcacgtccagacgtgcctggtcgcgccggtgaaggggcggtccttggtactgttttcttgggtgtgactaatactgtgcgtatgagcctcgatacgattggtcgcatcggggctacattcgaacaacgtacccgccgctatattttacgttccacgacgagttttttagtccgtttcggagcgcgttttttctttcgtgtagcatcagtacgagtacgtctatttccgtccattcgaggccttggtggtgtctttttctgtggtaaaaagagagtttgtacagacgcccaaagacgtatttgtgttttattttattgtttttttgtaaatttgtaatcttcggacagaatgttgtcctttttgttgggaatattcgtattctgcctgatgccggtacggccgcactggcatttttttgaccagtggcactaatttttcgtggtggctgtcgaggagccagtacaggcctcagtattacattctgagccatttgtgtagtccaactaagtgtagccagtcacgatttttcgctggctttatcgggcccccttatgtatgggcgtccgcatctctgttgtaatgtcgggggtatcctccatattgttttaattatcctctctttttgtgtatttgtggactttttgggaggacggatccgttttatcgtgggagggcgaggtcttgttgtatgtcgcgaaggtgtcgccgtcggtattgtcagtcggaatggtcattttttcttttggataatttttttgtggtgagctgtgccgtggtcgagttagtcagtgtcacattttttcccggttcacgtctcgctcatatatatcctgattttttactgcattgccaatttcaggtgttttttgtgggtcttttggcgtgcgcttggatgcgggtctttgctttcggttttttgggtgttgaaggagtttagcagtgaaggcaaaagggtgcaatgcagtgaagggtaaaattcttttgatgttaagggttgtgtatgttcaatgaggcgggattttggatgcagtgggcggggcaagggtgcggggcgcggtgcagtgtttgaggtgggggagtaatagtataaccgaagttaggttttattccatataataactactacaattaattcttaagcctagacgctgcgctccgacctaccggaaggggtaatactaagaagagcgaaggccgccgtagccctacgggcgcaacgtccggtacgacaggtccgtccatctactgctggtagtccctgtcgaagttccggtcgttttccggtccttggcatttttccggcgcaacgaccgcaaaaaggtatccgaggcggggggactgctcgtagtgtttttagctgcgagttcagtctccaccgctttgggctgtcctgatatttctatggtccgcaaagggggaccttcgagggagcacgcgagaggacaaggctgggacggcgaatggcctatggacaggcggaaagagggaagcccttcgcaccgcgaaagagtatcgagtgcgacatccatagagtcaagccacatccagcaagcgaggttcgacccgacacacgtgcttggggggcaagtcgggctggcgacgcggaataggccattgatagcagaactcaggttgggccattctgtgctgaatagcggtgaccgtcgtcggtgaccattgtcctaatcgtctcgctccatacatccgccacgatgtctcaagaacttcaccaccggattgatgccgatgtgatcttcctgtcataaaccatagacgcgagacgacttcggtcaatggaagcctttttctcaaccatcgagaactaggccgtttgtttggtggcgaccatcgccaccaaaaaaacaaacgttcgtcgtctaatgcgcgtctttttttcctagagttcttctaggaaactagaaaagatgccccagactgcgagtcaccttgcttttgagtgcaattccctaaaaccagtactctaatagtttttcctagaagtggatctaggaaaatttagttagatttcatatatactcatttgaaccagactgtcaatggttacgaattagtcactccgtggatagagtcgctagacagataaagcaagtaggtatcaacggactgaggggcagcacatctattgatgctatgccctcccgaatggtagaccggggtcacgacgttactatggcgctctgggtgcgagtggccgaggtctaaatagtcgttatttggtcggtcggccttcccggctcgcgtcttcaccaggacgttgaaataggcggaggtaggtcagataattaacaacggcccttcgatctcattcatcaagcggtcaattatcaaacgcgttgcaacaacggtaacgatgtccgtagcaccacagtgcgagcagcaaaccataccgaagtaagtcgaggccaagggttgctgttccgctcaatgtactagggggtacaacacgttttttcgccaatcgagggaagccaggaggctagcaacagtcttcattcaaccggcgtcacaatagtgagtaccaataccgtcgtgacgtattaagagaatgacagtacggtaggcattctacgaaaagacactgaccactcatgagttggttcagtaagactcttatcacatacgccgctggctcaacgagaacgggccgcagttgtgccctattatggcgcggtgtatcgtcttgaaattttcacgagtagtaaccttttgcaagaagccccgcttttgagagttcctagaatggcgacaactctaggtcaagctacattgggtgagcacgtgggttgactagaagtcgtagaaaatgaaagtggtcgcaaagacccactcgtttttgtccttccgttttacggcgttttttcccttattcccgctgtgcctttacaacttatgagtatgagaaggaaaaagttataataacttcgtaaatagtcccaataacagagtactcgcctatgtataaacttacataaatctttttatttgtttatccccaaggcgcgtgtaaaggggcttttcacggtggactgcagattctttggtaataatagtactgtaattggatatttttatccgcatagtgctccgggaaagcagaagttcttaacctaggcttaagaattaaagaattaatt

SEQ ID NO: 32 - DNA, 1479 bp, Artificial Sequence
  Codon optimized DNA encoding a tPA-gag fusion open reading frame
atggatgcaatgaagagagggctctgctgtgtgctgctgctgtgtggagcagtcttcgtttcgcccagcgagatctccattgtgtgggcctccagggagctggagaggtttgctgtgaaccctggcctgctggagacctctgaggggtgcaggcagatcctgggccagctccagccctccctgcaaacaggctctgaggagctgaggtccctgtacaacacagtggctaccctgtactgtgtgcaccagaagattgatgtgaaggacaccaaggaggccctggagaagattgaggaggagcagaacaagtccaagaagaaggcccagcaggctgctgctggcacaggcaactccagccaggtgtcccagaactaccccattgtgcagaacctccagggccagatggtgcaccaggccatctccccccggaccctgaatgcctgggtgaaggtggtggaggagaaggccttctcccctgaggtgatccccatgttctctgccctgtctgagggtgccaccccccaggacctgaacaccatgctgaacacagtggggggccatcaggctgccatgcagatgctgaaggagaccatcaatgaggaggctgctgagtgggacaggctgcatcctgtgcacgctggccccattgcccccggccagatgagggagcccaggggctctgacattgctggcaccacctccaccctccaggagcagattggctggatgaccaacaacccccccatccctgtgggggaaatctacaagaggtggatcatcctgggcctgaacaagattgtgaggatgtactcccccacctccatcctggacatcaggcagggccccaaggagcccttcagggactatgtggacaggttctacaagaccctgagggctgagcaggcctcccaggaggtgaagaactggatgacagagaccctgctggtgcagaatgccaaccctgactgcaagaccatcctgaaggccctgggccctgctgccaccctggaggagatgatgacagcctgccagggggtggggggccctggtcacaaggccagggtgctggctgaggccatgtcccaggtgaccaactccgccaccatcatgatgcagaggggcaacttcaggaaccagaggaagacagtgaagtgcttcaactgtggcaaggtgggccacattgccaagaactgtagggcccccaggaagaagggctgctggaagtgtggcaaggagggccaccagatgaaggactgcaatgagaggcaggccaacttcctgggcaaaatctggccctcccacaagggcaggcctggcaacttcctccagtccaggcctgagcccacagcccctcccgaggagtccttcaggtttggggaggagaagaccacccccagccagaagcaggagcccattgacaaggagctgtaccccctggcctccctgaggtccctgtttggcaacgacccctcctcccag

SEQ ID NO: 33 - protein, 493 aa, Artificial Sequence
  tPA-gag fusion open reading frame
MDAMKRGLCCVLLLCGAVFVSPSEISIVWASRELERFAVNPGLLETSEGCRQILGQLQPSLQTGSEELRSLYNTVATLYCVHQKIDVKDTKEALEKIEEEQNKSKKKAQQAAAGTGNSSQVSQNYPIVQNLQGQMVHQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKTVKCFNCGKVGHIAKNCRAPRKKGCWKCGKEGHQMKDCNERQANFLGKIWPSHKGRPGNFLQSRPEPTAPPEESFRFGEEKTTPSQKQEPIDKELYPLASLRSLFGNDPSSQ

SEQ ID NO: 34 - DNA, 4053 bp, Artificial Sequence
  Codon optimized DNA encoding a gag-IA pol fusion
atgggtgctagggcttctgtgctgtctggtggtgagctggacaagtgggagaagatcaggctgaggcctggtggcaagaagaagtacaagctaaagcacattgtgtgggcctccagggagctggagaggtttgctgtgaaccctggcctgctggagacctctgaggggtgcaggcagatcctgggccagctccagccctccctgcaaacaggctctgaggagctgaggtccctgtacaacacagtggctaccctgtactgtgtgcaccagaagattgatgtgaaggacaccaaggaggccctggagaagattgaggaggagcagaacaagtccaagaagaaggcccagcaggctgctgctggcacaggcaactccagccaggtgtcccagaactaccccattgtgcagaacctccagggccagatggtgcaccaggccatctccccccggaccctgaatgcctgggtgaaggtggtggaggagaaggccttctcccctgaggtgatccccatgttctctgccctgtctgagggtgccaccccccaggacctgaacaccatgctgaacacagtggggggccatcaggctgccatgcagatgctgaaggagaccatcaatgaggaggctgctgagtgggacaggctgcatcctgtgcacgctggccccattgcccccggccagatgagggagcccaggggctctgacattgctggcaccacctccaccctccaggagcagattggctggatgaccaacaacccccccatccctgtgggggaaatctacaagaggtggatcatcctgggcctgaacaagattgtgaggatgtactcccccacctccatcctggacatcaggcagggccccaaggagcccttcagggactatgtggacaggttctacaagaccctgagggctgagcaggcctcccaggaggtgaagaactggatgacagagaccctgctggtgcagaatgccaaccctgactgcaagaccatcctgaaggccctgggccctgctgccaccctggaggagatgatgacagcctgccagggggtggggggccctggtcacaaggccagggtgctggctgaggccatgtcccaggtgaccaactccgccaccatcatgatgcagaggggcaacttcaggaaccagaggaagacagtgaagtgcttcaactgtggcaaggtgggccacattgccaagaactgtagggcccccaggaagaagggctgctggaagtgtggcaaggagggccaccagatgaaggactgcaatgagaggcaggccaacttcctgggcaaaatctggccctcccacaagggcaggcctggcaacttcctccagtccaggcctgagcccacagcccctcccgaggagtccttcaggtttggggaggagaagaccacccccagccagaagcaggagcccattgacaaggagctgtaccccctggcctccctgaggtccctgtttggcaacgacccctcctcccagatggctcccatctcccccattgagactgtgcctgtgaagctgaagcctggcatggatggccccaaggtgaagcagtggcccctgactgaggagaagatcaaggccctggtggaaatctgcactgagatggagaaggagggcaaaatctccaagattggccccgagaacccctacaacacccctgtgtttgccatcaagaagaaggactccaccaagtggaggaagctggtggacttcagggagctgaacaagaggacccaggacttctgggaggtgcagctgggcatcccccaccccgctggcctgaagaagaagaagtctgtgactgtgctggctgtgggggatgcctacttctctgtgcccctggatgaggacttcaggaagtacactgccttcaccatcccctccatcaacaatgagacccctggcatcaggtaccagtacaatgtgctgccccagggctggaagggctcccctgccatcttccagtcctccatgaccaagatcctggagcccttcaggaagcagaaccctgacattgtgatctaccagtacatggctgccctgtatgtgggctctgacctggagattgggcagcacaggaccaagattgaggagctgaggcagcacctgctgaggtggggcctgaccacccctgacaagaagcaccagaaggagccccccttcctgtggatgggctatgagctgcaccccgacaagtggactgtgcagcccattgtgctgcctgagaaggactcctggactgtgaatgacatccagaagctggtgggcaagctgaactgggcctcccaaatctaccctggcatcaaggtgaggcagctgtgcaagctgctgaggggcaccaaggccctgactgaggtgatccccctgactgaggaggctgagctggagctggctgagaacagggagatcctgaaggagcctgtgcatggggtgtactatgacccctccaaggacctgattgctgagatccagaagcagggccagggccagtggacctaccaaatctaccaggagcccttcaagaacctgaagactggcaagtatgccaggatgaggggggcccacaccaatgatgtgaagcagctgactgaggctgtgcagaagatcaccactgagtccattgtgatctggggcaagacccccaagttcaagctgcccatccagaaggagacctgggagacctggtggactgagtactggcaggccacctggatccctgagtgggagtttgtgaacaccccccccctggtgaagctgtggtaccagctggagaaggagcccattgtgggggctgagaccttctatgtggctggggctgccaacagggagaccaagctgggcaaggctggctatgtgaccaacaggggcaggcagaaggtggtgaccctgactgacaccaccaaccagaagactgccctccaggccatctacctggccctccaggactctggcctggaggtgaacattgtgactgcctcccagtatgccctgggcatcatccaggcccagcctgatcagtctgagtctgagctggtgaaccagatcattgagcagctgatcaagaaggagaaggtgtacctggcctgggtgcctgcccacaagggcattgggggcaatgagcaggtggacaagctggtgtctgctggcatcaggaaggtgctgttcctggatggcattgacaaggcccaggatgagcatgagaagtaccactccaactggagggctatggcctctgacttcaacctgccccctgtggtggctaaggagattgtggcctcctgtgacaagtgccagctgaagggggaggccatgcatgggcaggtggactgctcccctggcatctggcagctggcctgcacccacctggagggcaaggtgatcctggtggctgtgcatgtggcctccggctacattgaggctgaggtgatccctgctgagacaggccaggagactgcctacttcctgctgaagctggctggcaggtggcctgtgaagaccatccacactgccaatggctccaacttcactggggccacagtgagggctgcctgctggtgggctggcatcaagcaggagtttggcatcccctacaacccccagtcccagggggtggtggcctccatgaacaaggagctgaagaagatcattgggcaggtgagggaccaggctgagcacctgaagacagctgtgcagatggctgtgttcatccacaacttcaagaggaaggggggcatcgggggctactccgctggggagaggattgtggacatcattgccacagacatccagaccaaggagctccagaagcagatcaccaagatccagaacttcagggtgtactacagggactccaggaaccccctgtggaagggccctgccaagctgctgtggaagggggagggggctgtggtgatccaggacaactctgacatcaaggtggtgcccaggaggaaggccaagatcatcagggactatggcaagcagatggctggggatgactgtgtggcctccaggcaggatgaggactaa

SEQ ID NO: 35 - protein, 1350 aa, Artificial Sequence
  Codon optimized gag-IA pol fusion
MGARASVLSGGELDKWEKIRLRPGGKKKYKLKHIVWASRELERFAVNPGLLETSEGCRQILGQLQPSLQTGSEELRSLYNTVATLYCVHQKIDVKDTKEALEKIEEEQNKSKKKAQQAAAGTGNSSQVSQNYPIVQNLQGQMVHQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKTVKCFNCGKVGHIAKNCRAPRKKGCWKCGKEGHQMKDCNERQANFLGKIWPSHKGRPGNFLQSRPEPTAPPEESFRFGEEKTTPSQKQEPIDKELYPLASLRSLFGNDPSSQMAPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLAVGDAYFSVPLDEDFRKYTAFTIPSINNETPGIRYQYNVLPQGWKGSPAIFQSSMTKILEPFRKQNPDIVIYQYMAALYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVAGAANRETKLGKAGYVTNRGRQKVVTLTDTTNQKTALQAIYLALQDSGLEVNIVTASQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLACTHLEGKVILVAVHVASGYIEAEVIPAETGQETAYFLLKLAGRWPVKTIHTANGSNFTGATVRAACWWAGIKQEFGIPYNPQSQGVVASMNKELKKIIGQVRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 36 - DNA, 64 bp, Artificial Sequence
  Nucleotide sequence at junction between nef coding sequence and plasmid backbone of nef expression vector V1Jns/nef
caccccgagtactacaaggactgctaaagcccgggcagatctgctgtgccttctagttgccagc

SEQ ID NO: 37 - DNA, 64 bp, Artificial Sequence; identical to SEQ ID NO: 36
  Nucleotide sequence at junction between nef coding sequence and plasmid backbone of nef expression vector V1Jns/modified nef

SEQ ID NO: 38 - DNA, 64 bp, Artificial Sequence; identical to SEQ ID NO: 36
  Nucleotide sequence at junction between nef coding sequence and plasmid backbone of nef expression vector V1Jns/modified nef fused to a leader sequence

(19) United States
(12) Patent Application Publication
(10) Pub. No.: US 2007/0077257 A1
(43) Pub. Date: Apr. 5, 2007

(54) Enhanced first generation adenovirus vaccines expressing condon optimized HIV1-Gag, Pol, Nef and modifications

(76) Inventors: Emilio A. Emini, Wayne, PA (US); Rima Youil, North Wales, PA (US); Andrew J. Bett, Lansdale, PA (US); Ling Chen, Blue Bell, PA (US); David C. Kaslow, Bryn Mawr, PA (US); John W. Shiver, Doylestown, PA (US); Timothy J. Toner, Marlton, NJ (US); Danilo R. Casimiro, Harleysville, PA (US)

Correspondence Address: MERCK AND CO., INC, P O BOX 2000, RAHWAY, NJ, 07065-0907, US

(21) Appl. No.: 11/604,553

(22) Filed: Nov. 27, 2006

Related U.S. Application Data
(63) Continuation of application No. 10/636,730, filed on Aug. 7, 2003.

(30) Foreign Application Priority Data
Sep. 14, 2001 (WO) PCT/US01/28861

(51) Int. Cl. A61K 39/00; A61K 39/12

(52) U.S. Cl. 424/204.1; 424/192.1

(57) ABSTRACT

First generation adenoviral vectors and-recombinant adenovirus-based HIV vaccines which contain HIV-1 gag, HIV-1 pol and/or HIV-1 nef polynucleotide pharmaceutical products, and biologically relevant modifications thereof are described. The adenovirus vaccines, when directly introduced into living vertebrate tissue, express the relevant proteins, inducing a cellular immune response which specifically recognizes HIV-1. The exemplified polynucleotides of the present invention are synthetic DNA molecules encoding HIV-1 Gag, HIV-1 Pol, HIV-1 Nef, and derivatives thereof. The adenoviral vaccines of the present invention, alone or in combination, will offer a prophylactic advantage to previously uninfected individuals and/or provide a therapeutic effect by reducing viral load levels within an infected individual, thus prolonging the asymptomatic phase of HIV-1 infection.

### CROSS-REFERENCE TO RELATED APPLICATIONS

This application is a continuation of U.S. application Ser. No. 10/636,730, filed Aug. 7, 2003, which claims priority under 35 U.S.C. §119(e) to U.S. Provisional Application Ser. Nos. 60/233,180, 60/279,056, and 60/317,814, filed Sep. 15, 2000, March 27, 2001, and Sep. 7, 2001, respectively.

### STATEMENT REGARDING FEDERALLY-SPONSORED R&D

Not Applicable

### REFERENCE TO MICROFICHE APPENDIX

Not Applicable

### FIELD OF THE INVENTION

The present invention relates to recombinant, replication-deficient first generation adenovirus vaccines found to exhibit enhanced growth properties and greater cellular-mediated immunity as compared to other replication-deficient vectors. The invention also relates to the associated first generation adenoviral vectors described herein, which, through the incorporation of additional 5′ adenovirus sequence, enhance large scale production efficiency of the recombinant, replication-defective adenovirus described herein. Another aspect of the instant invention is the surprising discovery that the intron A portion of the human cytomegalovirus (hCMV) promoter constitutes a region of instability in adenoviral vector constructs. Removal of this region from adenoviral expression constructs results in greatly improved vector stability. Therefore, improved vectors expressing a transgene under the control of an intron A-deleted CMV promoter constitute a further aspect of this invention. These adenoviral vectors are useful for generating recombinant adenovirus vaccines against human immunodeficiency virus (HIV). In particular, the first generation adenovirus vectors disclosed herein are utilized to construct and generate adenovirus-based HIV-1 vaccines which contain HIV-1 Gag, HIV-1 Pol and/or HIV-1 Nef polynucleotide pharmaceutical products, and biologically active modifications thereof. Host administration of the recombinant, replication-deficient adenovirus vaccines described herein results in expression of HIV-1 Gag, HIV-1-Pol and/or Nef protein or immunologically relevant modifications thereof, inducing a cellular immune response which specifically recognizes HIV-1. The exemplified polynucleotides of the present invention are synthetic DNA molecules encoding codon optimized HIV-1 Gag, HIV-1 Pol, derivatives of optimized HIV-1 Pol (including constructs wherein protease, reverse transcriptase, RNAse H and integrase activity of HIV-1 Pol is inactivated), HIV-1 Nef, and derivatives of optimized HIV-1 Nef, including nef mutants which effect wild type characteristics of Nef, such as myristylation and down regulation of host CD4. The HIV adenovirus vaccines of the present invention, when administered alone or in a combined modality and/or prime/boost regimen, will offer a prophylactic advantage to previously uninfected individuals and/or provide a therapeutic effect by reducing viral load levels within an infected individual, thus prolonging the asymptomatic phase of HIV-1 infection.

### BACKGROUND OF THE INVENTION

Human Immunodeficiency Virus-1 (HIV-1) is the etiological agent of acquired human immune deficiency syndrome (AIDS) and related disorders. HIV-1 is an RNA virus of the Retroviridae family and exhibits the 5′ LTR-gag-pol-env-LTR 3′ organization of all retroviruses. The integrated form of HIV-1, known as the provirus, is approximately 9.8 Kb in length. Each end of the viral genome contains flanking sequences known as long terminal repeats (LTRs). The HIV genes encode at least nine proteins and are divided into three classes; the major structural proteins (Gag, Pol, and Env), the regulatory proteins (Tat and Rev); and the accessory proteins (Vpu, Vpr, Vif and Nef).

The gag gene encodes a 55-kilodalton (kDa) precursor protein (p55) which is expressed from the unspliced viral mRNA and is proteolytically processed by the HIV protease, a product of the pol gene. The mature p55 protein products are p17 (matrix), p24 (capsid), p9 (nucleocapsid) and p6.

The pol gene encodes proteins necessary for virus replication; a reverse transcriptase, a protease, integrase and RNAse H. These viral proteins are expressed as a Gag-Pol fusion protein, a 160 kDa precursor protein which is generated via a ribosomal frame shifting. The viral encoded protease proteolytically cleaves the Pol polypeptide away from the Gag-Pol fusion and further cleaves the Pol polypeptide to the mature proteins which provide protease (Pro, P10), reverse transcriptase (RT, P50), integrase (IN, p31) and RNAse H (RNAse, p15) activities.

The nef gene encodes an early accessory HIV protein (Nef) which has been shown to possess several activities such as down regulating CD4 expression, disturbing T-cell activation and stimulating HIV infectivity.

The env gene encodes the viral envelope glycoprotein that is translated as a 160-kilodalton (kDa) precursor (gp160) and then cleaved by a cellular protease to yield the external 120-kDa envelope glycoprotein (gp120) and the transmembrane 41-kDa envelope glycoprotein (gp41). Gp120 and gp41 remain associated and are displayed on the viral particles and the surface of HIV-infected cells.

The tat gene encodes a long form and a short form of the Tat protein, a RNA binding protein which is a transcriptional transactivator essential for HIV-1 replication.

The rev gene encodes the 13 kDa Rev protein, a RNA binding protein. The Rev protein binds to a region of the viral RNA termed the Rev response element (RRE). The Rev protein promotes transfer of unspliced viral RNA from the nucleus to the cytoplasm. The Rev protein is required for HIV late gene expression and in turn, HIV replication.

Gp 120 binds to the CD4/chemokine receptor present on the surface of helper T-lymphocytes, macrophages and other target cells in addition to other co-receptor molecules. X4 (macrophage tropic) virus show tropism for CD4/CXCR4 complexes while a R5 (T-cell line tropic) virus interacts with a CD4/CCR5 receptor complex. After gp 120 binds to CD4, gp41 mediates the fusion event responsible for virus entry. The virus fuses with and enters the target cell, followed by reverse transcription of its single stranded RNA genome into the double-stranded DNA via a RNA dependent DNA polymerase. The viral DNA, known as provirus, enters the cell nucleus, where the viral DNA directs the production of new viral RNA within the nucleus, expression of early and late HIV viral proteins, and subsequently the production and cellular release of new virus particles. Recent advances in the ability to detect viral load within the host shows that the primary infection results in an extremely high generation and tissue distribution of the virus, followed by a steady state level of virus (albeit through a continual viral production and turnover during this phase), leading ultimately to another burst of virus load which leads to the onset of clinical AIDS. Productively infected cells have a half life of several days, whereas chronically or latently infected cells have a 3-week half life, followed by non-productively infected cells which have a long half life (over 100 days) but do not significantly contribute to day to day viral loads seen throughout the course of disease.

Destruction of CD4 helper T lymphocytes, which are critical to immune defense, is a major cause of the progressive immune dysfunction that is the hallmark of HIV infection. The loss of CD4 T-cells seriously impairs the body's ability to fight most invaders, but it has a particularly severe impact on the defenses against viruses, fungi, parasites and certain bacteria, including mycobacteria.

Effective treatment regimens for HIV-1 infected individuals have become available recently. However, these drugs will not have a significant impact on the disease in many parts of the world and they will have a minimal impact in halting the spread of infection within the human population. As is true of many other infectious diseases, a significant epidemiologic impact on the spread of HIV-1 infection will only occur subsequent to the development and introduction of an effective vaccine. There are a number of factors that have contributed to the lack of successful vaccine development to date. As noted above, it is now apparent that in a chronically infected person there exists constant virus production in spite of the presence of anti-HIV-1 humoral and cellular immune responses and destruction of virally infected cells. As in the case of other infectious diseases, the outcome of disease is the result of a balance between the kinetics and the magnitude of the immune response and the pathogen replicative rate and accessibility to the immune response. Pre-existing immunity may be more successful with an acute infection than an evolving immune response can be with an established infection. A second factor is the considerable genetic variability of the virus. Although anti-HIV-1 antibodies exist that can neutralize HIV-1 infectivity in cell culture, these antibodies are generally virus isolate-specific in their activity. It has proven impossible to define serological groupings of HIV-1 using traditional methods. Rather, the virus seems to define a serological “continuum” so that individual neutralizing antibody responses, at best, are effective against only a handful of viral variants. Given this latter observation, it would be useful to identify immunogens and related delivery technologies that are likely to elicit anti-HIV-1 cellular immune responses. It is known that in order to generate CTL responses antigen must be synthesized within or introduced into cells, subsequently processed into small peptides by the proteasome complex, and translocated into the endoplasmic reticulum/Golgi complex secretory pathway for eventual association with major histocompatibility complex (MHC) class I proteins. CD8⁺ T lymphocytes recognize antigen in association with class I MHC via the T cell receptor (TCR) and the CD8 cell surface protein. Activation of naive CD8⁺ T cells into activated effector or memory cells generally requires both TCR engagement of antigen as described above as well as engagement of costimulatory proteins. Optimal induction of CTL responses usually requires “help” in the form of cytokines from CD4⁺ T lymphocytes which recognize antigen associated with MHC class II molecules via TCR and CD4 engagement.

European Patent Applications 0 638 316 (Published Feb. 15, 1995) and 0 586 076 (Published Mar. 9, 1994), (both assigned to American Home Products Corporation) describe replicating adenovirus vectors carrying an HIV gene, including env or gag. Various treatment regimens were used with chimpanzees and dogs, some of which included booster adenovirus or protein plus alum treatments.

Replication-defective adenoviral vectors harboring deletions in the E1 region are known, and recent adenoviral vectors have incorporated the known packaging repeats into these vectors; e.g., see EP 0 707 071, disclosing, inter alia, an adenoviral vector deleted of E1 sequences from base pairs 459 to 3328; and U.S. Pat. No. 6,033,908, disclosing, inter alia, an adenoviral vector deleted of base pairs 459-3510. The packaging efficiency of adenovirus has been taught to depend on the number of incorporated individual A (packaging) repeats; see, e.g., Gräble and Hearing, 1990 J. Virol. 64(5):2047-2056; Gräble and Hearing, 1992 J. Virol. 66(2):723-731.

Larder, et al., (1987, Nature 327: 716-717) and Larder, et al., (1989, Proc. Natl. Acad. Sci. 86: 4803-4807) disclose site specific mutagenesis of HIV-1 RT and the effect such changes have on in vitro activity and infectivity related to interaction with known inhibitors of RT.

Davies, et al. (1991, Science 252:, 88-95) disclose the crystal structure of the RNase H domain of HIV-1 Pol.

Schatz, et al. (1989, FEBS Lett. 257: 311-314) disclose that mutations Glu478Gln and His539Phe in a complete HIV-1 RT/RNase H DNA fragment results in defective RNase activity without effecting RT activity.

Mizrahi, et al. (1990, Nucl. Acids. Res. 18: pp. 5359-5353) disclose additional mutations Asp443Asn and Asp498Asn in the RNase region of the pol gene which also results in defective RNase activity. The authors note that the Asp498Asn mutant was difficult to characterize due to instability of this mutant protein.

Leavitt, et al. (1993, J. Biol. Chem. 268: 2113-2119) disclose several mutations, including a Asp64Val mutation, which show differing effect on HIV-1 integrase (IN) activity.

Wiskerchen, et al. (1995, J. Virol. 69: 376-386) disclose singe and double mutants, including mutation of aspartic acid residues which effect HIV-1 IN and viral replication functions.

It would be of great import in the battle against AIDS to produce a prophylactic- and/or therapeutic-based HIV vaccine which generates a strong cellular immune response against an HIV infection. The present invention addresses and meets these needs by disclosing a class of adenovirus vaccines which, upon host administration, express codon optimized and modified versions of the HIV-1 genes, gag, pol and nef These recombinant, replication-defective adenovirus vaccines may be administered to a host, such as a human, alone or as part of a combined modality regimen and/or prime-boost vaccination regimen with components of the present invention and/or a distinct viral HIV DNA vaccine, non-viral HIV DNA vaccine, HIV subunit vaccine, an HIV whole killed vaccine and/or a live attenuated HIV vaccine.

### SUMMARY OF THE INVENTION

The present invention relates to enhanced replication-defective recombinant adenovirus vaccine vectors and associated recombinant, replication-deficient adenovirus vaccines which encode various forms of HIV-1 Gag, HIV-1 Pol, and/or HIV-1 Nef, including immunologically relevant modifications of HIV-1 Gag, HIV-1 Pol and HIV-1 Nef. The adenovirus vaccines of the present invention express HIV antigens and provide for improved cellular-mediated immune responses upon host administration. Potential vaccinees include but are not limited to primates and especially humans and non-human primates, and also include any non-human mammal of commercial or domestic veterinary importance. An effect of the improved recombinant adenovirus-based vaccines of the present invention should be a lower transmission rate to previously uninfected individuals (i.e., prophylactic applications) and/or reduction in the levels of the viral loads within an infected individual (i.e., therapeutic applications), so as to prolong the asymptomatic phase of HIV-1 infection. In particular, the present invention relates to adenoviral-based vaccines which encode various forms of codon optimized HIV-1 Gag (including but in no way limited to p55 versions of codon optimized full length (FL) Gag and tPA-Gag fusion proteins), HIV-1 Pol, HIV-1 Nef, and selected modifications of immunological relevance. The administration, intracellular delivery and expression of these adenovirus vaccines elicit a host CTL and Th response. The preferred replication-defective recombinant adenoviral vaccine vectors include but are not limited to synthetic DNA molecules which (1) encode codon optimized versions of wild type HIV-1 Gag; (2) encode codon optimized versions of HIV-1 Pol; (3) encode codon optimized versions of HIV-1 Pol fusion proteins; (4) encode codon optimized versions of modified HIV-1 Pol proteins and fusion proteins, including but not limited to pol modifications involving residues within the catalytic regions responsible for RT, RNase and IN activity within the host cell; (5) encode codon optimized versions of wild type HIV-1 Nef; (6) codon optimized versions of HIV-1 Nef fusion proteins; and/or (7) codon optimized versions of HIV-1 Nef derivatives, including but not limited to nef modifications involving introduction of an amino-terminal leader sequence, removal of an amino-terminal myristylation site and/or introduction of dileucine motif mutations. The Nef-based fusion and modified proteins, disclosed within this specification and expressed from an adenoviral-based vector vaccine this specification, may possess altered trafficking and/or host cell function while retaining the ability to be properly presented to the host MHC I complex and in turn elicit a host CTL and Th response. Examples of HIV-1 Gag, Pol and/or Nef fusion proteins include but are not limited to fusion of a leader or signal peptide at the NH₂-teriminal portion of the viral antigen coding region. Such a leader peptide includes but is not limited to a tPA leader peptide.

The adenoviral vector utilized in construction of the HIV-1 Gag-, HIV-1 Pol- and/or HIV-1 Nef-based vaccines of the present invention may comprise any replication-defective adenoviral vector which provides for enhanced genetic stability of the recombinant adenoviral genome through large scale production and purification of the recombinant virus. In other words, an HIV-1 Gag-, Pol- or Nef-based adenovirus vaccine of the present invention is a purified recombinant, replication-defective adenovirus which is shown to be genetically stable through multiple passages in cell culture and remains so during large scale production and purification procedures. Such a recombinant adenovirus vector and harvested adenovirus vaccine lends itself to large scale dose filling and subsequent worldwide distribution procedures which will be demanded of an efficacious monovalent or multivalent HIV vaccine. The present invention meets this basic requirement with description of a replication-defective adenoviral vector and vectors derived therefrom, at least partially deleted in E1, comprising a wildtype adenovirus cis-acting packaging region from about base pair 1 to between from about base pair 342 (more preferably, 400) to about base pair 458 of the wildtype adenovirus genome. A preferred embodiment of the instant invention comprises base pairs 1-450 of a wildtype adenovirus. In other preferred embodiments, the replication-defective adenoviral vector has, in addition thereto, a region 3′ to the E1-deleted region comprising base pairs 3511-3523. Basepairs 342-450 (more particularly, 400-450) constitute an extension of the 5′region of previously disclosed vectors carrying viral antigens, particularly HIV antigens (see, e.g., PCT International Application PCT/US00/18332, published Jan. 11, 2001 (WO 01/02067), which claims priority to U.S. Provisional Application Ser. Nos. 60/142,631 and 60/148,981, filed Jul. 6, 1999 and Aug. 13, 1999, respectively; these documents herein incorporated by reference. Applicants have found that extending the 5′ region further into the E1 gene into the disclosed vaccine vectors incorporated elements found to be important in optimizing the packaging of the virus.

As compared to previous vectors not comprising basepairs from about 1 to between from about base pair 342 (more preferably, 400) to about base pair 458 of the wildtype adenovirus genome, vectors comprising the above region exhibited enhanced growth characteristics, with approximately 5-10 fold greater amplification rates, a more potent virus effect, allowing lower doses of virus to be used to generate equivalent immunity; and a greater cellular-mediated immune response than replication-deficient vectors not comprising this region (basepairs 1-450). Even more important, adenoviral constructs derived therefrom are very stable genetically in large-scale production, particularly those comprising an expression cassette under the control of a hCMV promoter devoid of intron A. This is because Applicants have surprisingly found that the intron A portion of the hCMV promoter constituted a region of instability when employed in adenoviral vectors. Applicants have, therefore, identified an enhanced adenoviral vector which is particularly suited for use in gene therapy and nucleotide-based vaccine vectors which, favorably, lends itself to large scale propagation.

A preferred embodiment of this invention is a replication-defective adenoviral vector in accordance with the above description wherein the gene is inserted in the form of a gene expression cassette comprising (a) a nucleic acid encoding a protein or biologically active and/or immunologically relevant portion thereof; (b) a heterologous promoter operatively linked to the nucleic acid of part a); and, (c) a transcription terminator.

In preferred embodiments, the E1 gene, other than that contained within basepairs 1450 or, alternatively, that contained within base pairs 1-450 and 3511-3523 has been deleted from the adenoviral vector, and the gene expression cassette has replaced the deleted E1 gene. In other preferred embodiments, the replication defective adenovirus genome does not have a functional E3 gene, or the E3 gene has been deleted. Most preferably, the E3 region is present within the adenoviral genome. Further preferred embodiments are wherein the gene expression cassette is in an E1 anti-parallel (transcribed in a 3′ to 5′ direction relative to the vector backbone) orientation or, more preferably, an E1 parallel (transcribed in a 5′ to 3′ direction relative to the vector backbone) orientation.

Further embodiments relate to a shuttle plasmid vector comprising: an adenoviral portion and a plasmid portion, wherein said adenovirus portion comprises: a) a replication defective adenovirus genome, at least partially deleted in E1, comprising a wildtype adenovirus cis-acting packaging region from about base pair 1 to between from about base pair 342 (more preferably, 400) to about base pair 458 (preferably, 1-450) of the wildtype adenovirus genome and, preferably, in addition thereto, basepairs 3511-3523 of a wildtype adenovirus sequence; and b) a gene expression cassette comprising: (a) a nucleic acid encoding a protein or biologically active and/or immunologically relevant portion thereof; (b) a heterologous promoter operatively linked to the nucleic acid of part a);and (c) a transcription terminator and/or a polyadenylation site.

Other aspects of this invention include a host cell comprising said adenoviral vectors and/or said shuttle plasmid vectors; vaccine compositions comprising said vectors; and methods of producing the vectors comprising (a) introducing the adenoviral vector into a host cell which expresses adenoviral E1 protein, and (b) harvesting the resultant adenoviral vectors.

To this end, the present invention particularly relates to harvested recombinant, replication defective virus derived from a host cell, such as but not limited to 293 cells or PER.C6® cells, including but not limited to harvested virus related to any of the MRKAd5 vector backbones, with or without an accompanying transgene, including but not limited to the HIV-1 antigens described herein. An HIV-1 vaccine is represented by any harvested, recombinant adenovirus material which expresses any one or more of the HIV-1 antigens disclosed herein. This harvested material may then be purified, formulated and stored prior to host administration.

Another aspect of this invention is a method of generating a cellular immune response against a protein in an individual comprising administering to the individual an adenovirus vaccine vector comprising:

a) a recombinant, replication defective adenoviral vector, at least partially deleted in E1, comprising a wildtype adenovirus cis-acting adenovirus packaging region from about base pair 1 to between from about base pair 342 (more preferably, 400) to about base pair 458 (preferably, 1-450) and, preferably in addition thereto, base pairs 3511-3523 of a wildtype adenovirus sequence, and,

b) a gene expression cassette comprising:(i) a nucleic acid encoding a protein or biologically active and/or immunologically relevant portion thereof; (ii) a heterologous promoter operatively linked to the nucleic acid of part a); and (iii) a transcription terminator and/or a polyadenylation site.

In view of the efficacious nature of the adenoviral and/or DNA plasmid vaccines described herein, the present invention relates to all methodology regarding administration of one or more of these adenoviral and/or DNA plasmid vaccines to provide effective immunoprophylaxis, to prevent establishment of an HIV-1 infection following exposure to this virus, or as a post-HIV infection therapeutic vaccine to mitigate the acute HIV-1 infection so as to result in the establishment of a lower virus load with beneficial long term consequences. As discussed herein, such a treatment regimen may include a monovalent or multivalent composition, various combined modality applications, and/or a prime/boost regimen to as to optimize antigen expression and a concomitant cellular-mediated and/or humoral immune response upon inoculation into a living vertebrate tissue. Therefore, the present invention provides for methods of using the adenoviral and/or DNA plasmid vaccines disclosed herein within the various parameters disclosed herein as well as any additional parameters known in the art, which, upon introduction into mammalian tissue induces intracellular expression of the gag, pol and/or nef-based vaccines.

To this end, the present invention relates in part to methods of generating a cellular immune response in a vaccinee, preferably a human vaccinee, wherein the individual is given more than one administration of adenovirus vaccine vector, and it may be given in a regimen accompanied by the administration of a plasmid vaccine. The plasmid vaccine (also referred to herein as a “DNA plasmid vaccine” or “vaccine plasmid” comprises a nucleic acid encoding a protein or an immunologically relevant portion thereof, a heterologous promoter operably linked to the nucleic acid sequence, and a transcription terminator or a polyadenylation signal (such as bGH or SPA, respectively). There may be a predetermined minimum amount of time separating the administrations. The individual can be given a first dose of plasmid vaccine, and then a second dose of plasmid vaccine. Alternatively, the individual may be given a first dose of adenovirus vaccine, and then a second dose of adenovirus vaccine. In other embodiments, the plasmid vaccine is administered first, followed after a time by administration of the adenovirus vaccine. Conversely, the adenovirus vaccine may be administered first, followed by administration of plasmid vaccine after a time. In these embodiments, an individual may be given multiple doses of the same adenovirus serotype in either viral vector or plasmid form, or the virus may be of differing serotypes. In the alternative, a viral antigen of interest can be first delivered via a viral vaccine other than an adenovirus-based vaccine, and then followed with the adenoviral vaccine disclosed. Alternative viral vaccines include but are not limited to pox virus and venezuelan equine encephilitis virus.

The present invention also relates to multivalent adenovirus vaccine compositions which comprise Gag, Pol and Nef components described herein; see, e.g., Example 29 and Table 25. Such compositions will provide for an enhanced cellular immune response subsequent to host administration, particularly given the genetic diversity of human MHCs and of circulating virus. Examples, but not limitations, include MRKAd5 -vector based multivalent vaccine compositions which provide for a divalent (i.e., gag and nef, gag and pol, or pol and nef components) or a trivalent vaccine (i.e., gag, pol and nef components) composition. Such a mutlivalent vaccine may be filled for a single dose or may consist of multiple inoculations of each individually filled component; and may in addition be part of a prime/boost regimen with viral or non-viral vector vaccines as introduced in the previous paragraph. To this end, preferred compositions are MRKAd5 adenovirus used in combination with multiple, distinct HIV antigen classes. Each HIV antigen class is subject to sequence manipulation, thus providing for a multitude of potential vaccine combinations; and such combinations are within the scope of the present invention. The utilization of such combined modalities vaccine formulation and administration increase the probability of eliciting an even more potent cellular immune response when compared to inoculation with a single modality regimen.

The concept of a “combined modality” as disclosed herein also covers the alternative mode of administration whereby multiple HIV-1 viral antigens may be ligated into a proper shuttle plasmid for generation of a pre-adenoviral plasmid comprising multiple open reading frames. For example, a trivalent vector may comprise a gag-pol-nef fusion, in either a E3(−) or E3(+) background, preferably a E3 deleted backbone, or possibly a “2+1” divalent vaccine, such as a gag-pol fusion (i.e., codon optimized p55 gag and inactivated optimized pol; Example 29 and Table 25) within the same MRKAd5 backbone, with each open reading frame being operatively linked to a distinct promoter and transcription termination sequence. Alternatively, the two open reading frames may be operatively linked to a single promoter, with the open reading frames operatively linked by an internal ribosome entry sequence (IRES). Therefore, a multivalent vaccine delivered as a single, or possibly a second harvested recombinant, replication-deficient adenovirus is contemplated as part of the present invention.

Therefore, the adenoviral vaccines and plasmid DNA vaccines of this invention may be administered alone, or may be part of a prime and boost administration regimen. A mixed modality priming and booster inoculation scheme will result in an enhanced immune response, particularly if pre-existing anti-vector immune responses are present. This one aspect of this invention is a method of priming a subject with the plasmid vaccine by administering the plasmid vaccine at least one time, allowing a predetermined length of time to pass, and then boosting by administering the adenoviral vaccine. Multiple primings typically, 1-4, are usually employed, although more may be used. The length of time between priming and boost may typically vary from about four months to a year, but other time frames may be used. In experiments with rhesus monkeys, the animals were primed four times with plasmid vaccines, then were boosted 4 months later with the adenoviral vaccine. Their cellular immune response was notably higher than that of animals which had only received adenoviral vaccine. The use of a priming regimen may be particularly preferred in situations where a person has a pre-existing anti-adenovirus immune response.

It is an object of the present invention to provide for enhanced replication-defective recombinant adenoviral vaccine vector backbones. These recombinant adenoviral backbones may accept one or more transgenes, which may be passaged through cell culture for growth, amplification and harvest.

It is a further object to provide for enhanced replication-defective recombinant adenoviral vaccine vectors which encode various transgenes.

It is also an object of the present invention to provide for a harvested recombinant, replication-deficient adenovirus which shows enhanced growth and amplification rates while in combination with increased virus stability after continuous passage in cell culture. Such a recombinant adenovirus is particularly suited for use in gene therapy and nucleotide-based vaccine vectors which, favorably, lends itself to large scale propagation.

To this end, it is an object of the present invention to provide for (1) enhanced replication-defective recombinant adenoviral vaccine vectors as described herein which encode various forms of HIV-1 Gag, HIV-1 Pol, and/or HIV-1 Nef, including immunologically relevant modifications of HIV-1 Gag, HIV-1 Pol and HIV-1 Nef, and (2) harvested, purified recombinant replication-deficient adenovirus generated by passage of the adenoviral vectors of (1) through one or multiple passages through cell culture, including but not limited to passage through 293 cells or PER.C6® cells.

It is also an object of the present invention to provide for recombinant adenovirus harvested by one or multiple passages through cell culture. As relating to recombinant adenoviral vaccine vector, this recombinant virus is harvested and formulated for subsequent host administration.

It is also an object of the present invention to provide for replication-defective adenoviral vectors wherein at least one gene is inserted in the form of a gene expression cassette comprising (a) a nucleic acid encoding a protein or biologically active and/or immunologically relevant portion thereof; (b) a heterologous promoter operatively linked to the nucleic acid of part a); and, (c) a transcription terminator.

It is also an object of the present invention to provide for a host cell comprising said adenoviral vectors and/or said shuttle plasmid vectors; vaccine compositions comprising said vectors; and methods of producing the vectors comprising (a) introducing the adenoviral vector into a host cell which expresses adenoviral E1 protein, and (b) harvesting the resultant adenoviral vectors.

It is a further object of the present invention to provide for methods of generating a cellular immune response against a protein in an individual comprising administering to the individual an adenovirus vaccine vector comprising a) a replication defective adenoviral vector, at least partially deleted in E1, comprising a wildtype adenovirus cis-acting packaging region from about base pair 1 to between from about base pair 342 (more preferably, 400) to about 450 (preferably, 1-450) and, preferably, 3511-3523 of a wildtype adenovirus sequence, and, b) a gene expression cassette comprising:(i) a nucleic acid encoding a protein or biologically active and/or immunologically relevant portion thereof; (ii) a heterologous promoter operatively linked to the nucleic acid of part a); and (iii) a transcription terminator and/or a polyadenylation site.

It is also an object of the present invention to provide various alternatives for vaccine administration regimes, namely administration of one or more adenoviral and/or DNA plasmid vaccines described herein to provide effective immunoprophylaxis for uninfected individuals or a therapeutic treatment for HIV infected patients. Such processes include but are not limited to multivalent HIV-1 vaccine compositions, various combined modality regimes as well as various prime/boost alternatives. These methods of administration, relating to vaccine composition and/or scheduled administration, will increase the probability of eliciting an even more potent cellular immune response when compared to inoculation with a single modality regimen.

As used throughout the specification and claims, the following definitions and abbreviations are used:

“HAART” refers to—highly active antiretroviral therapy—.

“first generation” vectors are characterized as being replication-defective. They typically have a deleted or inactivated E1 gene region, and preferably have a deleted or inactivated E3 gene region as well.

“AEX” refers to Anion Exchange chromatography.

“QPA” refers to Quick PCR-based Potency Assay.

“bps” refers to basepairs.

“s” or “str” denotes that the transgene is in the E1 parallel or “straight⇄ orientation.

“PBMCs” refers to peripheral blood monocyte cells.

“FL” refers to full length.

“FLgag” refers to a full-length optimized gag gene, as shown in FIG. 2.

“Ad5-Flgag” refers to an adenovirus serotype 5 replication deficient virus which carries an expression cassette which comprises a full length optimized gag gene under the control of a CMV promoter.

“Promoter” means a recognition site on a DNA strand to which an RNA polymerase binds. The promoter forms an initiation complex with RNA polymerase to initiate and drive transcriptional activity. The complex can be modified by activating sequences such as enhancers or inhibiting sequences such as silencers.

“Leader” means a DNA sequence at the 5′ end of a structural gene which is transcribed along with the gene. This usually results a protein having an N-terminal peptide extension, often referred to as a pro-sequences.

“Intron” means a section of DNA occurring in the middle of a gene which does not code for an amino acid in the gene product. The precursor RNA of the intron is excised and is therefore not transcribed into mRNA not translated into protein.

“Immunologically relevant” or “biologically active” means (I) with regards to a viral protein, that the protein is capable, upon administration, of eliciting a measurable immune response within an individual sufficient to retard the propagation and/or spread of the virus and/or to reduce the viral load present within the individual; or (2) with regards to a nucleotide sequence, that the sequence is capable of encoding for a protein capable of the above.

“Cassette” refers to a nucleic acid sequence which is to be expressed, along with its transcription and translational control sequences. By changing the cassette, a vector can express a different sequence.

“bGHpA” refers to the bovine growth hormone transcription terminator/polyadenylation sequence.

“tPAgag” refers to a fusion between the leader sequence of the tissue plasminogen activator leader sequence and an optimized HIV gag gene, as exemplified in FIGS. 30A-1 to 30A-2, whether in a DNA or adenovirus-based vaccine vector.

Where utilized, “IA” or “inact” refers to an inactivated version of a gene (e.g. IApol).

“MCS” is “multiple cloning site”.

In general, adenoviral constructs, gene constructs are named by reference to the genes contained therein. For example:

“Ad5 HIV-1 gag”, also referred to as the original HIV-1 gag adenoviral vector, is a vector containing a transgene cassette composed of a hCMV intron A promoter, the full length version of the human codon-optimized HIV-1 gag gene, and the bovine growth hormone polyadenylation signal. The transgene was inserted in the E1 antiparallel orientation in an E1 and E3 deleted adenovector.

“MRK Ad5 HIV-1 gag” also referred to as “MRKAd5gag” or “Ad5gag2” is an adenoviral vector taught herein which is deleted of E1 comprises basepairs 1-450 and 3511-3523, and has a human codon-optimized HIV-1 gene in an E1 parallel orientation under the control of a CMV promoter without intron A. The construct also comprises a bovine growth hormone polyadenylation signal.

“pV1JnsHIVgag”, also referred to as “HIVFLgagPR9901”, is a plasmid comprising the CMV immediate-early (IE) promoter and intron A, a full-length codon-optimized HIV gag gene, a bovine growth hormone-derived polyadenylation and transcriptional termination sequence, and a minimal pUC backbone.

“pV1JnsCMV(no intron)-FLgag-bGHpA” is a plasmid derived from pV1JnsHIVgag which is deleted of the intron A portion of CMV and which comprises the full length HIV gag gene. This plasmid is also referred to as “pV1JnsHIVgag-bGHpA”, pV1Jns-hCMV-FL-gag-bGHpA” and “pV1JnsCMV(no intron)+FLgag+bGHpA”.

“pV1JnsCMV(no intron)-FLgag-SPA” is a plasmid of the same composition as pV1JnsCMV(no intron)-FLgag-bGHpA except that the SPA termination sequence replaces that of bGHpA. This plasmid is also referred to as “pV1Jns-HIVgag-SPA” and pV1Jns-hCMV-FLgag-SPA”.

“pdelE1sp1A” is a universal shuttle vector with no expression cassette (i.e., no promoter or polyA). The vector comprises wildtype adenovirus serotype 5 (Ad5) sequences from bp 1 to bp 341 and bp 3524 to bp 5798, and has a multiple cloning site between the Ad5sequences ending 341 bp and beginning 3524 bp. This plasmid is also referred to as the original Ad 5 shuttle vector. “MRKpdelE1sp1A” or “MRKpde1E1(Pac/pDXpack450)” or “MRKpdelE1(Pac/pIX/pack450)Cla1” is a universal shuttle vector with no expression cassette (i.e. no promoter or polyA) comprising wildtype adenovirus serotype 5 (Ad5) sequences from bp1 to bp450 and bp 3511 to bp 5798. The vector has a multiple cloning site between the Ad5 sequence ending 450 bp and beginning 3511 bp. This shuttle vector may be used to insert the CMV promoter and the bGHpA fragments in both the straight (“str”. or E1 parallel) orientation or in the opposite (opp. or E1 antiparallel) orientation)

“MRKpdelE1(Pac/pD(/pack450)+CMVmin+BGHpA(str.)” is still another shuttle vector which is the modified vector that contains the CMV promoter (no intronA) and the bGHpA fragments. The expression unit containing the hCMV promoter (no intron A) and the bovine growth hormone polyadenylation signal has been inserted into the shuttle vector such that insertion of the gene of choice at a unique Bg/II site will ensure the direction of transcription of the transgene will be Ad5 E1 parallel when inserted into the MRKpAd5(E1/E3+)C1a1 pre-plasmid. This shuttle vector, as shown in FIGS. 22 and 23, was used to insert the respective IApol and G2A,LLAA nef genes directly into.

“MRKpdelE1-CMV(no intron)-FLgag-bGHpA” is a shuttle comprising Ad5 sequences from basepairs 1-450 and 3511-5798, with an expression cassette containing human CMV without intron A, the full-length human codon-optimized HIV gag gene and bovine growth hormone polyadenylation signal. This plasmid is also referred to as “MRKpdelE1 shuttle+hCMV-FL-gag-BGHpA”

“MRKpAdHVE3+CMV(no intron)-FLgag-bGHpA” is an adenoviral vector comprising all Ad5 sequences except those nucleotides encompassing the E1 region (from 451-3510), a human CMV promoter without intron A, a full-length human codon-optimized HIV gag gene, and a bovine growth hormone polyadenylation signal. This vector is also referred to as “MRKpAdHVE3 +hCMV-FL-gag-BGHpA”, “MRKpAd5HIV-1gag”, “MRKpAd5gag”, “pMRKAd5gag” or “pAd5gag2”.

“pV1Jns-HIV-pol inact(opt)” or “pV1Jns-HIV IA pol (opt) is the inactivated Pol gene (contained within SEQ ID NO:3) cloned into the BglII site of V1Jns (FIGS. 17A-1 to 17A-3). As noted herein, various derivatives of HIV-1 pol may be cloned into a plasmid expression vector such as V1Jns or V1Jns-tPA, thus serving directly as DNA vaccine candidates or as a source for subcloning into an appropriate adenoviral vector.

“MRKpdel+hCMVmin+FL-pol+bGHpA(s)” is the “MRKpdelE1(Pac/pIX/pack450)+CMVmin+BGHpA(str.)” shuttle mentioned above which contains the IA pol gene is the proper orientation. This shuttle vector is used in a bacterial recombination with MRKpAd(E1−/E3+)Cla1.

37 MRKpAd+hCMVmin+FL-pol+bGHpA(S)E3+”, also referred to herein as “pMRKAd5pol”, is the pre-adenovirus plasmid which comprises a CMV-pol inact(opt)-pGHpA construct. The construction of this pre-adenovirus plasmid is shown in FIG. 22.

“pV1Jns/nef (G2A,LLAA)” or “V1Jns/opt nef (G2A,LLAA)” comprises codon optimized HIV-1 Nef wherein the open reading frame codes for modifications at the amino terminal myristylation site (Gly-2 to Ala-2) and substitution of the Leu-174-Leu-175 dileucine motif to Ala-174-Ala-175 (SEQ ID NO: 13; which comprises an initiating methionine residue at nucleotides 12-14 and a “TAA” stop codon from nucleotides 660-662). This fragment is subcloned into the Bgl II site of V1Jns and/or V1Jns-tPA (FIGS. 16A-B). As noted above for HIV-1 pol, HIV-1 nef constructs may be cloned into a plasmid expression vector such as V1Jns or V1Jns-tPA, thus serving directly as DNA vaccine candidates or as a source for subcloning into an appropriate adenoviral vector.

“MRKpdelE1hCMVminFL-nefBGHpA(s)”, also referred to herein as “pMRKAd5nef”, is the pre-adenovirus plasmid which comprises a CMV-nef (G2A,LLAA) codon optimized sequence. The construction of this pre-adenovirus plasmid is shown in FIG. 23.

### BRIEF DESCRIPTION OF THE FIGURES

FIG. 1 shows the original HIV-1 gag adenovector (Ad5HIV-1gag). This vector is disclosed in PCT International Application No. PCT/US00/18332 (WO 01/02607) filed Jul. 3, 2000, claiming priority to U.S. Provisional Application Ser. No. 60/142,631, filed Jul. 6, 1999 and U.S. Application Ser. No. 60/148,981, filed Aug. 13, 1999, all three applications which are hereby incorporated by reference.

FIG. 2 shows the nucleic acid sequence (SEQ ID NO: 27) of the optimized human HIV-1 gag open reading frame.

FIG. 3 shows diagrammatically the new transgene constructs in comparison with the original gag transgene.

FIG. 4 shows the modifications made to the original adenovector backbone in the generation of the novel vectors of the instant invention.

FIG. 5 shows the virus mixing experiments that were carried out to determine the effects of the addition made to the packaging signal region (Expt. #1) and the E3 gene on viral growth (Expt. #2). The bars denote the region of modifications made to the E1 deletion.

FIGS. 6A-6B show an autoradiograph of viral DNA analysis following the viral mixing experiments described in Examples 6 and 7.

FIGS. 7A, 7B and 7C are as follows: FIG. 7A shows the hCMV-F1gag-bGHpA adenovectors constructed within the MRKpAdHVE3 and MRKpAdHVO adenovector backbones. Both E1 parallel and E1 antiparallel transgene orientation are represented. FIG. 7B shows the hCMV-Flgag-SPA adenovectors constructed within the MRKpAdHVE3 and MRKpAdHVO adenovector backbones. Again, both E1 parallel and E1 antiparallel transgene orientation are represented. FIG. 7C shows the mCMV-Flgag-bGHpA adenovectors constructed within the MRKpAdHVE3 and MRKpAdHVO adenovector backbones. Once again, both E1 parallel and E1 antiparallel transgene orientation are represented.

FIG. 8A shows the experiment designed to test the effect of transgene orientation.

FIG. 8B shows the experiments designed to test the effect of polyadenylation signal.

FIG. 9 shows viral DNA from the four adenoviral vectors tested (Example 12) at P5, following BstE 11 digestion.

FIG. 10 shows viral DNA analysis of passages 11 and 12 of MRKpAdHVE3, MRKAd5HIV-1gag, and MRKAd5HIV-1gagE3-.

FIG. 11 shows viral DNA analysis (HindIII digestion) of passage 6 MRKpAdHVE3 and MRKAd5HIV-1gag used to initiate the viral competition study. The last two lanes are passage 11 analysis of duplicate passages of the competition study (each virus at MOI of 280 viral particles).

FIG. 12 shows viral DNA analysis by Hind III digestion on high passage numbers for MRKAd5HIV-1gag in serum-containing media with collections made at specified times. The first lane shows the 1 kb DNA size marker. The other lanes represent pre-plasmid control (digested with Pac1 and HindIII, MRKAd5HIV-1gag at P16, P19, and P21.

FIG. 13 shows serum anti-p24 levels at 3 wks post i.m. immunization of balb/c mice (n=10) with varying doses of several Adgag constructs: (A) MRK Ad5 HIV-1 gag (through passage 5); (B) MRKAd5 hCMV-FLgag-bGHpA (E3-); (C) MRKAd5 hCMV-FLgag-SPA (E3+); (D) MRKAd5 mCMV-FLgag-bGHpA (E3+); (E) research lot (293 cell-derived) of Ad5HIV-1 gag; and (F) clinical lot (Ad5gagFN0001) of Ad5HIV-1 gag. Reported are the geometric mean titers (GMT) for each cohort along with the standard error bars.

FIG. 14 shows a restriction map of the pMRKAd5HIV-1gag vector.

FIGS. 15A-1 to 15A45 illustrate the nucleotide sequence of the pMRKAd5HIV-1gag vector (SEQ ID NO:25 [coding] and SEQ ID NO:26 [non-coding]).

FIGS. 16A-B shows a schematic representation of DNA vaccine expression vectors V1Jns (A) and V1Jns-tPA (B), which are utilized for HIV-1 gag, pol and nef constructs in various DNA/viral vector combined modality regimens as disclosed herein.

FIGS. 17A-1 to 17A-3 show the nucleotide (SEQ ID NO:3) and amino acid sequence (SEQ ID NO:4) of IA-Pol. Underlined codons and amino acids denote mutations, as listed in Table 1.

FIG. 18 shows codon optimized nucleotide and amino acid sequences through the fusion junction of tPA-pol inact(opt) (contained within SEQ ID NOs: 7 and 8, respectively). The underlined portion represents the NH₂-terminal region of IA-Pol.

FIGS. 19A-1 to 19A-2 show a nucleotide sequence comparison between wild type nefjrfl) and codon optimized nef. The wild type nef gene from the jrfl isolate consists of 648 nucleotides capable of encoding a 216 amino acid polypeptide. WT, wild type sequence (SEQ ID NO: 19); opt, codon-optimized sequence (contained within SEQ ID NO:9). The Nef amino acid sequence is shown in one-letter code (SEQ ID NO: 10).

FIGS. 20A-C show nucleotide sequences at junctions between nef coding sequence and plasmid backbone of nef expression vectors V1Jns/nef (FIG. 20A), V1Jns/nef(G2A,LLAA) (FIG. 20B), V1Jns/tpanef (FIG. 20C) and V1Jns/tpanef(LLAA) (FIG. 20C, also). 5′ and 3′ flanking sequences of codon optimized nef or codon optimized nef mutant genes are indicated by bold/italic letters; nef and nef mutant coding sequences are indicated by plain letters. Also indicated (as underlined) are the restriction endonuclease sites involved in construction of respective nef expression vectors. V1Jns/tpanef and V1Jns/tpanef(LLAA) have identical sequences at the junctions.

FIG. 21 shows a schematic presentation of nef and nef derivatives. Amino acid residues involved in Nef derivatives are presented. Glycine 2 and Leucine 174 and 175 are the sites involved in myristylation and dileucine motif, respectively. For both versions of the tpanef fusion genes, the putative leader peptide cleavage sites are indicated with “*”, and a exogenous serine residue introduced during the construction of the mutants is underlined.

FIG. 22 shows diagrammatically the construction of the pre-adenovirus plasmid construct, MRKAd5Pol.

FIG. 23 shows diagrammatically the construction of the pre-adenovirus plasmid construct, MRKAd5Nef.

FIG. 24 shows a comparison of clade B vs. clade C anti-gag T cell responses in clade B HIV-infected subjects.

FIG. 25 shows a comparison of clade B vs. clade C anti-nef T cell responses in clade B HIV-infected subjects.

FIGS. 26A-1 to 26A-46 illustrate the nucleotide sequence of the pMRKAd5HIV-1pol adenoviral vector (SEQ ID NO:28 [coding] and SEQ ID NO:29 [non-coding]), comprising the coding region of the inactivated pol gene (SEQ ID NO:3).

FIGS. 27A-1 to 27A-44 illustrate the nucleotide sequence of the pMRKAd5HIV-1 nef adenoviral vector (SEQ ID NO:30 [coding] and SEQ ID NO:31 [non-coding]), comprising the coding region of the inactivated nef gene (SEQ ID NO: 13).

FIG. 28 shows the stability of MRKAd5 vectors comprising various promoter fragments (hCMV or mCMV) and terminations signals (bGH or SPA) in E3(+) or E3(−) backbones.

FIGS. 29A and B shows the anion-exchange HPLC viral particle concentrations of the freeze-thaw recovered cell associated virus at the 24, 36, 48, and 60 hpi time points (FIG. 29A) and the timcourse QPA supernatant titers (FIG. 29B) for MRKAd5gag, MRKAd5pol and MRKAd5nef.

FIGS. 30A-1 to 30A-2 show the nucleotide sequence (SEQ ID NO:32) and amino acid sequence (SEQ ID NO:33) comprising the open reading frame of a representative tPA-gag fusion for use in the DNA and/or adenoviral vaccine disclosed herein.

FIG. 31 shows the intracellular yIFN staining of PBMCs collected at week 10 (post DNA prime) and week 30 (post Ad boost). The cells were stimulated overnight in the presence or absence of the gag peptide pool. They were subsequently stained using fluorescence-tagged anti-CD3, anti-CD8, anti-CD4, and anti-γIFN monoclonal antibodies. Each plot shows all CD3+ T cells which were segregated in terms of positive staining for surface CD8 and γIFN production. The numbers in the upper right and lower right quadrants of each plot are the percentages of CD3⁺ cells that were CD8⁺γIFN⁺ and CD4⁺γIFN⁺, respectively.

FIG. 32 shows a comparison of single-modality adenovirus immunization with DNA+adjuvant prime/adenovirus boost immunization.

FIGS. 33A-1 to 33A-2 show the nucleotide sequence (SEQ ID NO: 34) of the open reading frame for the gag-IApol fusion of Example 29.

FIGS. 34A-1 to 34A-2 show the protein sequence (SEQ ID NO:35) of the gag-IApol fusion frame.

### DETAILED DESCRIPTION OF THE INVENTION

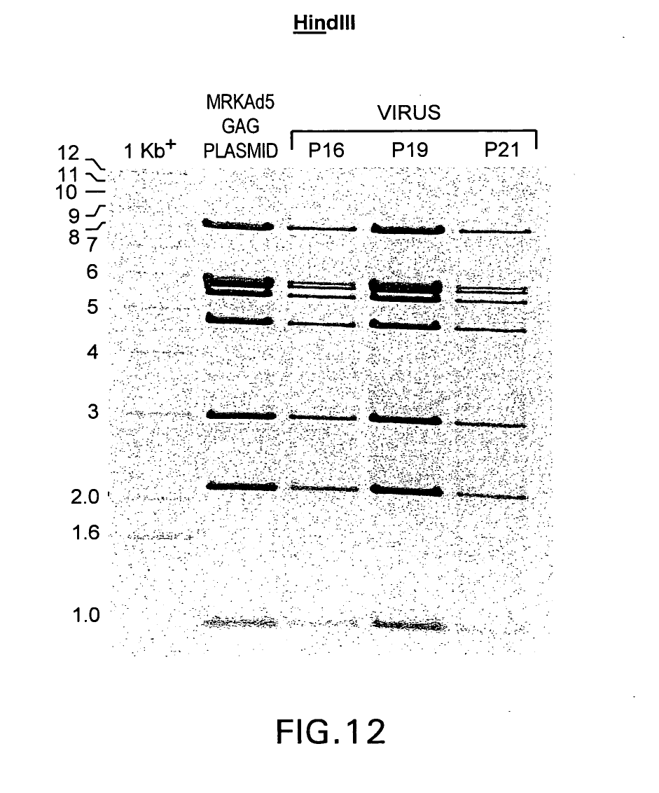

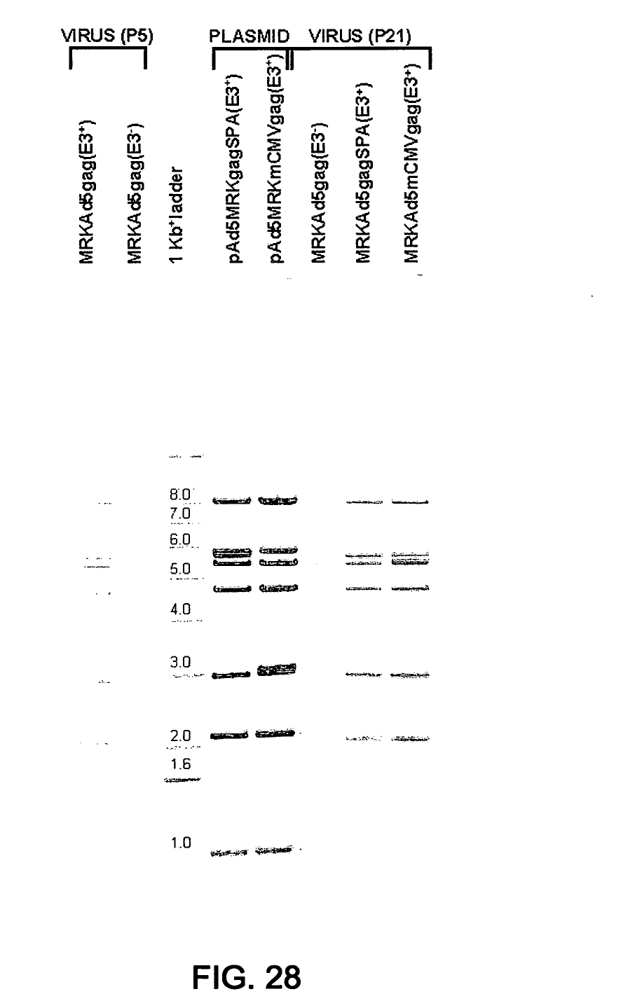

A novel replication-defective, or “first generation,” adenoviral vector suitable for use in gene therapy or nucleotide-based vaccine vectors is described. This vector is at least partially deleted in E1 and comprises a wildtype adenovirus cis-acting packaging region from about base pair 1 to between about base pair 342 (more preferably, 400) to about 458 (preferably, 1-450) and, preferably, 3511-3523 of a wild-type adenovirus sequence. It has been found that a vector of this description possesses enhanced growth characteristics, with approximately 5-10 fold greater amplification rates, and is more potent allowing lower doses of virus to be used to generate equivalent immunity. The vector, furthermore, generates a harvested recombinant adenovirus which shows greater cellular-mediated immune responses than replication-deficient vectors not comprising this region (basepairs 342-450). Adenoviral constructs derived from these vectors are, further, very stable genetically, particularly those comprising a transgene under the control of a hCMV promoter devoid of intron A. Viruses in accordance with this description were passaged continually and analyzed; see Example 12. Each virus analyzed maintained it correct genetic structure. Analysis was also carried out under propagation conditions similar to that performed in large scale production. Again, the vectors were found to possess enhanced genetic stability; see FIG. 12. Following 21 passages, the viral DNA showed no evidence of rearrangement, and was highly reproducible from one production lot to the next. The outcome of all relevant tests indicate that the adenoviral vector is extremely well suited for large-scale production of recombinant, replication-deficient adenovirus, as shown herein with the data associated with FIG. 28.

A preferred adenoviral vector in accordance with this description is a vector comprising basepairs 1-450, which is deleted in E3. This vector can accommodate up to approximately 7,500 base pairs of foreign DNA inserts (or exogenous genetic material). Another preferred vector is one retaining E3 which comprises basepairs 1-450. A preferred vector of this description is an E3+ vector comprising basepairs 1-450 and 3511-3523. This vector, when deleted of the region spanning basepairs451-3510, can accommodate up to approximately, 4,850 base pairs of foreign DNA inserts (or exogenous genetic material). The cloning capacities of the above vectors have been determined using 105% of the wildtype Ad5 sequence as the upper genome size limit.

Wildtype adenovirus serotype 5 is used as the basis for the specific basepair numbers provided throughout the specification. The wildtype adenovirus serotype 5 sequence is known and described in the art; see, Chroboczek et al., 1992 J. Virology 186:280, which is hereby incorporated by reference. Accordingly, a particular embodiment of the instant invention is a vector based on the adenovirus serotype 5 sequence. One of skill in the art can readily identify the above regions in other adenovirus serotypes (e.g., serotypes 2, 4, 6, 12, 16, 17, 24, 31, 33, and 42), regions defined by basepairs corresponding to the above basepair positions given for adenovirus serotype 5. Accordingly, the instant invention encompasses all adenoviral vectors partially deleted in E1 comprising basepairs corresponding to 1-450 (particularly, 342-450) and, preferably, 3511-3523 of a wild-type adenovirus serotype 5 (Ad5) nucleic acid sequence. Particularly preferred embodiments of the instant invention are those derived from adenoviruses like Ad5 which are classified in subgroup C (e.g., Ad2).

Vectors in accordance with the instant invention are at least partially deleted in E1. Preferably the E1 region is completely deleted or inactivated. Most preferably, the region deleted of E1 is within basepairs 451-3510. It is to be noted that the extended 5′ and 3′ regions of the disclosed vectors are believed to effectively reduce the size of the E1 deletion of previous constructs without overlapping any part of the E1A/E1B gene present in the cell line used, i.e., the PER.C6® cell line transefected with base pairs 459-3510. Overlap of adenoviral sequences is avoided because of the possibility of recombination. One of ordinary skill in the art can certainly appreciate that the instant invention can, therefore, be modified if a different cell line transfected with a different segment of adenovirus DNA is utilized. For purposes of exemplification, a 5′ region of base pairs 1 to up to 449 is more appropriate if a cell line is transfected with adenoviral sequence from base pairs 450-3510. This holds true as well in the consideration of segments 3′ to the E1 deletion.

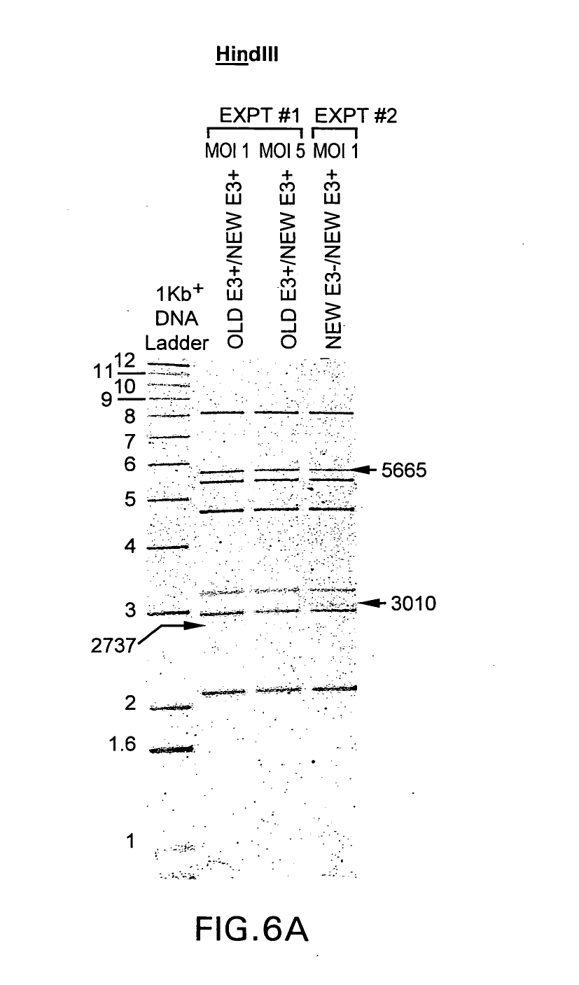

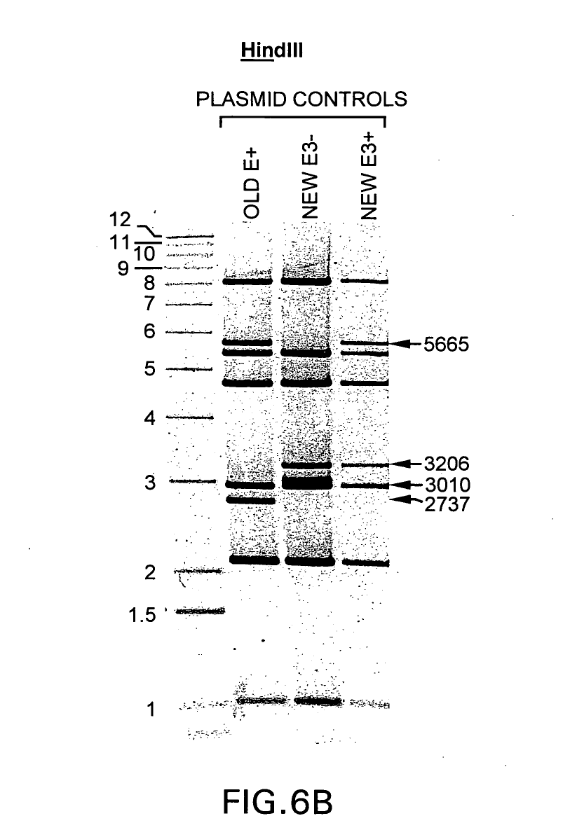

Preferred embodiments of the instant invention possess an intact E3 region (i.e., an E3 gene capable of encoding a functional E3). Alternate embodiments have a partially deleted E3, an inactivated E3 region, or a sequence completely deleted of E3. Applicants have found, in accordance with the instant invention, that virus comprising the E3 gene were able to amplify more rapidly compared with virus not comprising an E3 gene; see FIG. 6 wherein a diagnostic CsCl band corresponding to the E3+ virus tested (5,665 bp) was present in greater amount compared with the diagnostic band of 3,010 bp corresponding to the E3− virus. These results were obtained following a virus competition study involving mixing equal MOI ratio (1:1) of adenovectors both comprising the E3 gene and not comprising the E3 gene. This increased amplification capacity of the E3+ adenovectors was subsequently confirmed with growth studies; see Table 4A, wherein the E3+ virus exhibit amplification ratios of 470, 420 and 320 as compared with the 115 and 40-50 of the E3− constructs.

As stated above, vectors in accordance with the instant invention can accommodate up to approximately 4,850 base pairs of exogenous genetic material for an E3+ vector and approximately 7,500 base pairs for an E3− vector. Preferably, the insert brings the adenoviral vector as close as possible to a wild-type genomic size (e.g., for Ad5, 35,935 basepairs). It is well known that adenovirus amplifies best when they are close to their wild-type genomic size.

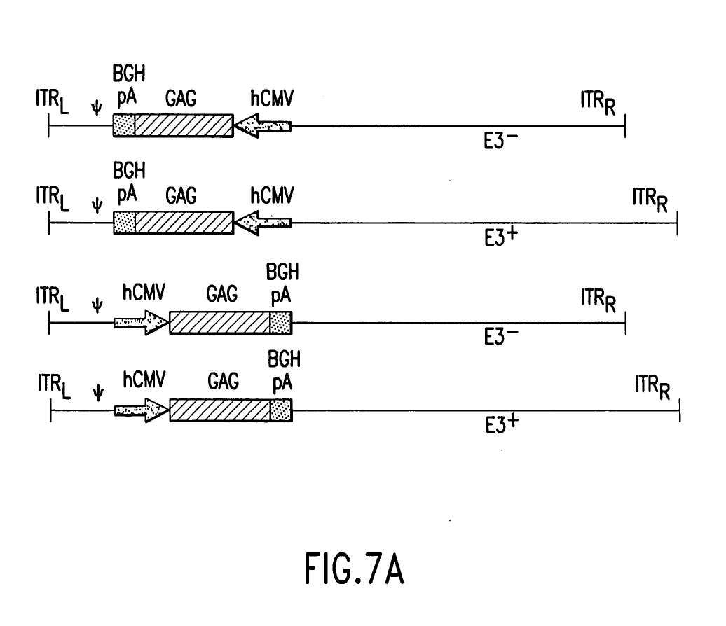

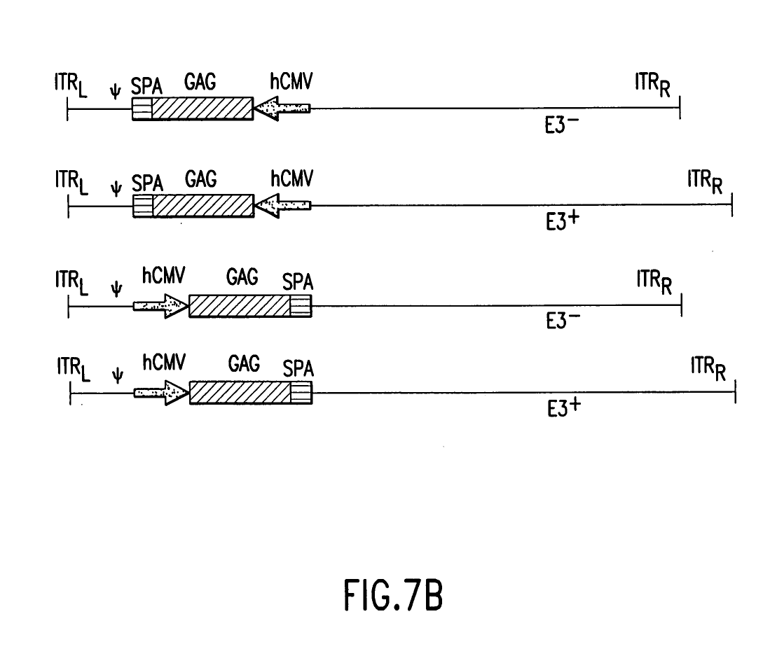

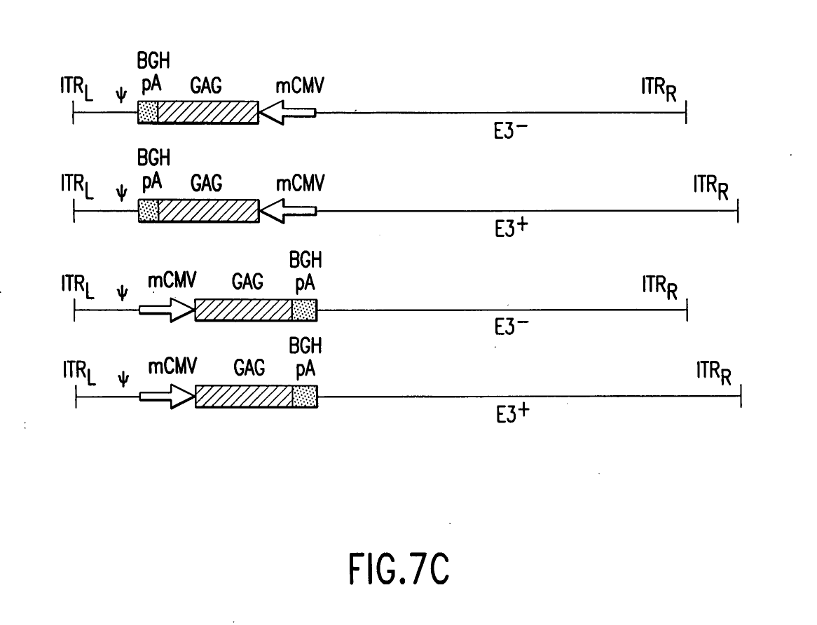

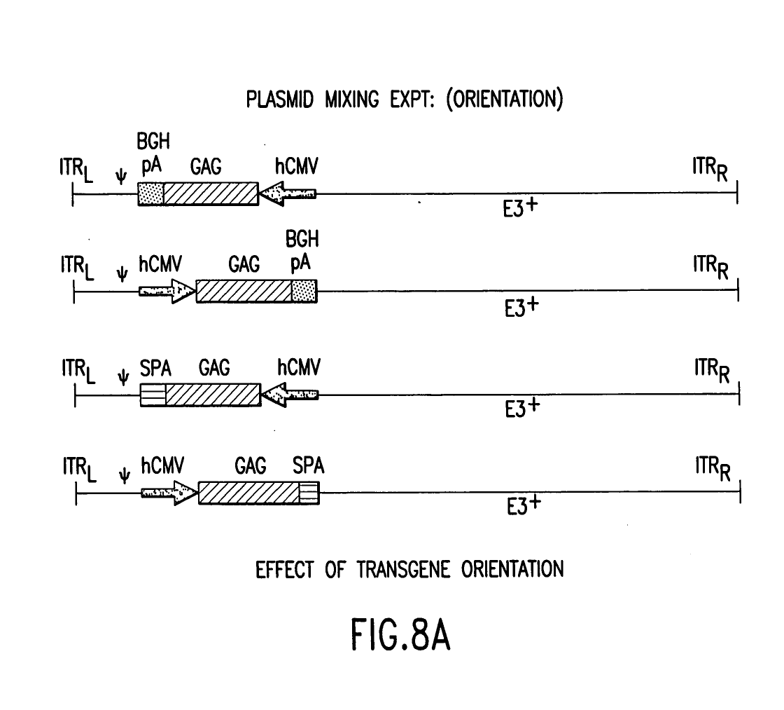

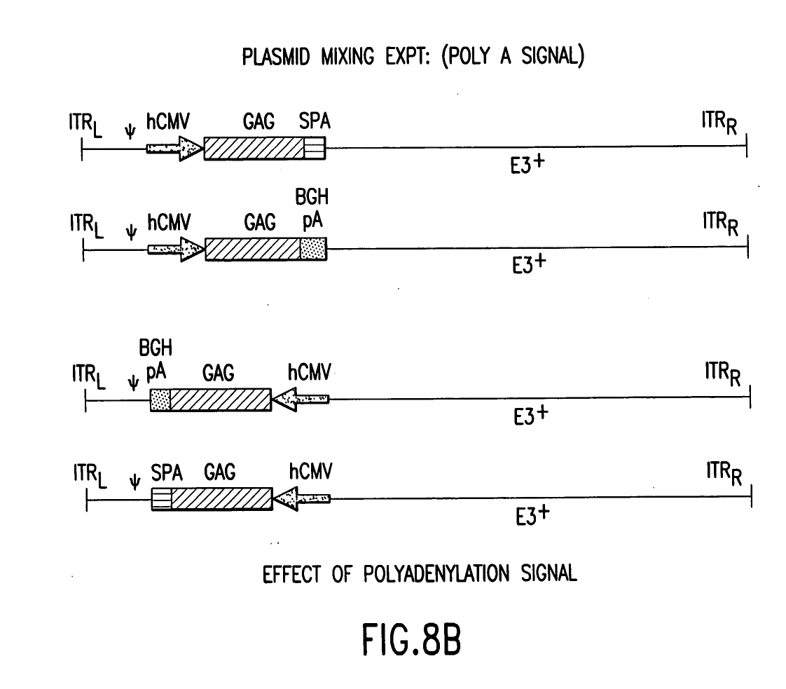

The genetic material can be inserted in an E1-parallel or an E1anti-parallel orientation, as such is illustrated in FIG. 7A, 7B, 7C and FIG. 8A. Particularly preferred embodiments of the instant invention, have the insert in an E1-parallel orientation. Applicants have found, via competition experiments with plasmids containing transgenes in differing orientation (FIG. 8A), that vector constructs with the foreign DNA insert in an E1-parallel orientation amplify better and actually out-compete E1-antiparallel-oriented transgenes. Viral DNA analysis of the mixtures at passage 3 and certainly at passage 6, showed a greater ratio of the virus carrying the transgene in the E1 parallel orientation as compared with the E1 anti-parallel version. By passage 10, the only viral species observed was the adenovector with the transgene in the E1 parallel orientation for both transgenes tested.

Adenoviral vectors in accordance with the instant invention are particularly well suited to effectuate expression of desired proteins, one example of which is an HIV protein, particularly an HIV full length gag protein. Exogenous genetic material encoding a protein of interest can exist in the form of an expression cassette. A gene expression cassette preferably comprises (a) a nucleic acid encoding a protein of interest; (b) a heterologous promoter operatively linked to the nucleic acid encoding the protein; and (c) a transcription terminator.

The transcriptional promoter is preferably recognized by an eukaryotic RNA polymerase. In a preferred embodiment, the promoter is a “strong” or “efficient” promoter. An example of a strong promoter is the immediate early human cytomegalovirus promoter (Chapman et al, 1991 Nucl. Acids Res19:3979-3986, which is incorporated by reference), preferably without intronic sequences. Most preferred for use within the instant adenoviral vector is a human CMV promoter without intronic seqeunces, like intron A. Applicants have found that intron A, a portion of the human cytomegalovirus promoter (hCMV), constitutes a region of instability for adenoviral vectors. CMV without intron A has been found to effectuate (Examples 1-3) comparable expression capabilities in vitro when driving H1V gag expression and, furthermore, behaved equivalently to intron A-containing constructs in Balb/c mice in vivo with respect to their antibody and T-cell responses at both dosages of plasmid DNA tested (20 μg and 200 μg). Those skilled in the art will appreciate that any of a number of other known promoters, such as the strong immunoglobulin, or other eukaryotic gene promoters may also be used, including the EF 1 alpha promoter, the murine CMV promoter, Rous sarcoma virus (RSV) promoter, SV40 early/late promoters and the beta-actin promoter.

In preferred embodiments, the promoter may also comprise a regulatable sequence such as the Tet operator sequence. This would be extremely useful, for example, in cases where the gene products are effecting a result other than that desired and repression is sought.

Preferred transcription termination sequences present within the gene expression cassette are the bovine growth hormone terminator/polyadenylation signal (bGHpA) and the short synthetic polyA signal (SPA) of 50 nucleotides in length, defined as follows:

(SEQ ID NO:18)

AATAAAAGATCTTTATTTTCATTAGATCTGTGTGTTGGT-
TTTTTGTGTG.

The combination of the CMV promoter (devoid of the intron A region) with the BGH terminator is particularly preferred although other promoter/terminator combinations in the context of FG adenovirus may also be used.

Other embodiments incorporate a leader or signal peptide into the transgene. A preferred leader is that from the tissue-specific plasminogen activator protein, tPA. Examples include but are not limited to the various tPA-gag, tPA-pol and tPA-nef adenovirus-based vaccines disclosed throughout this specification.

In view of the improved adenovirus vectors described herein, an essential portion of the present invention are adenoviral-based HIV vaccines comprising said adenovirus backbones which may be administered to a mammalian host, preferably a human host, in either a prophylactic or therapeutic setting. The HIV vaccines of the present invention, whether administered alone or in combination regimens with other viral- or non-viral-based DNA vaccines, should elicit potent and broad cellular immune responses against HIV that will either lessen the likelihood of persistent virus infection and/or lead to the establishment of a clinically significant lowered virus load subject to HIV infection or in combination with HAART therapy, mitigate the effects of previously established HIV infection (antiviral immunotherapy(ARI)). While any HIV antigen (e.g., gag, pol, nef, gp160, gp41, gp120, tat, rev, etc.) may be utilized in the herein described recombinant adenoviral vectors, preferred embodiments include the codon optimized p55 gag antigen (herein exemplified as MRKAd5gag), pol and nef. Sequences based on different Clades of HIV-1 are suitable for use in the instant invention, most preferred of which are Clade B and Clade C. Particularly preferred embodiments are those sequences (especially, codon-optimized sequences) based on concensus Clade B sequences. Preferred versions of the MRKAd5pol and MRKAd5nef series of adenoviral vaccines will encode modified versions of pol or nef, as discussed herein. Preferred embodiments of the MRKAd5HIV-1 vectors carrying HIV envelope genes and modifications thereof comprise the HIV codon-optimized env sequences of PCT International Applications PCT/US97/02294 and PCT/US97/10517, published Aug. 28, 1997 (WO 97/31115) and Dec. 24, 1997, respectively; both documents of which are hereby incorporated by reference.

A most preferred aspect of the instant invention is the disclosed use of the adenoviral vector described above to effectuate expression of HIV gag. Sequences for many genes of many HIV strains are publicly available in GENBANK and primary, field isolates of HIV are available from the National Institute of Allergy and Infectious Diseases (NIAID) which has contracted with Quality Biological (Gaithersburg, Md.) to make these strains available. Strains are also available from the World Health Organization (WHO), Geneva Switzerland. It is preferred that the gag gene be from an HIV-1 strain (CAM-1; Myers et al, eds. “Human Retroviruses and AIDS: 1995, IIA3-IIA19, which is hereby incorporated by reference). This gene closely resembles the consensus amino acid sequence for the clade B (North American/European) sequence. Therefore, it is within the purview of the skilled artisan to choose an appropriate nucleotide sequence which encodes a specific HIV gag antigen, or immunologically relevant portion thereof. As shown in Example 25, a clade B or clade C based p55 gag antigen will potentially be useful on a global scale. As noted herein, the transgene of choice for insertion in to a DNA or MRKAd-based adenoviral vector of the present invention is a codon optimized version of p55 gag. Such a MRKAd5gag adenoviral vector is documented in Example 11 and is at least referred to herein as MRKAd5HIV-1 gag. Of course, additional versions are contemplated, including but not limited to modifications such as promoter (e.g., mCMV for hCMV) and/or pA-terminations signal (SPA for bGH) switching, as well as generating MRK Ad5 backbones with or without deletion of the Ad5 E3 gene.

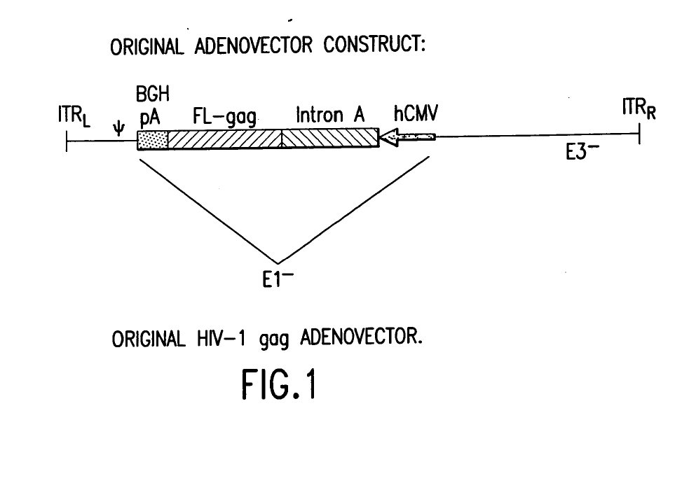

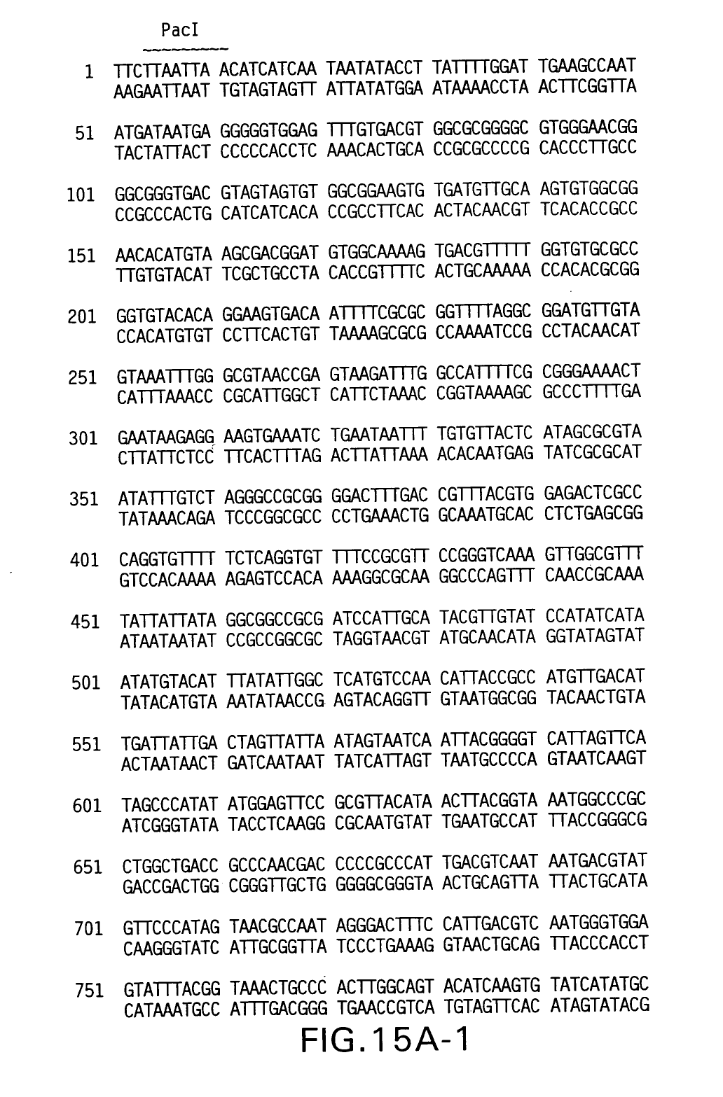

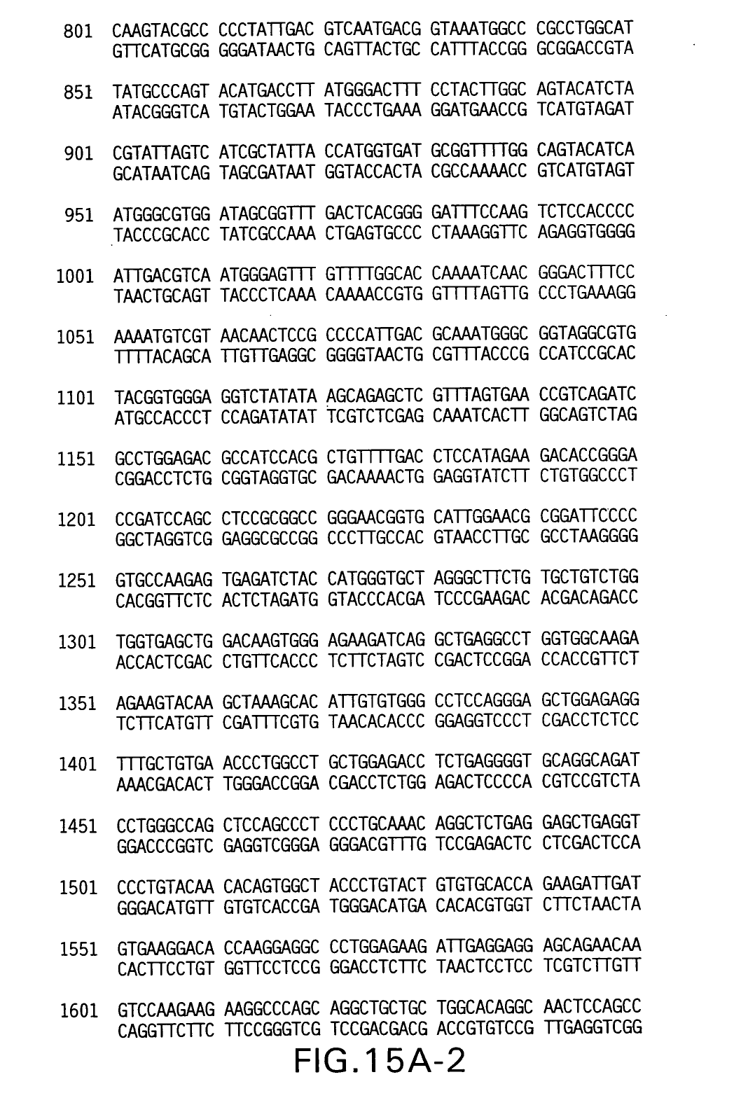

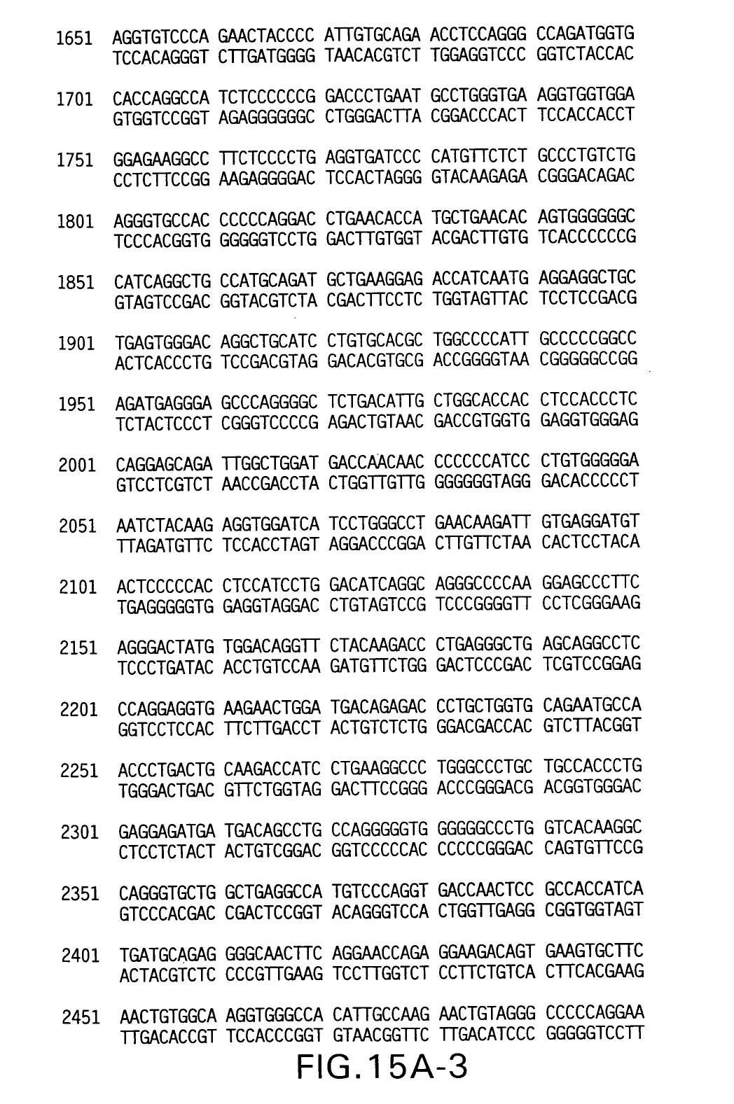

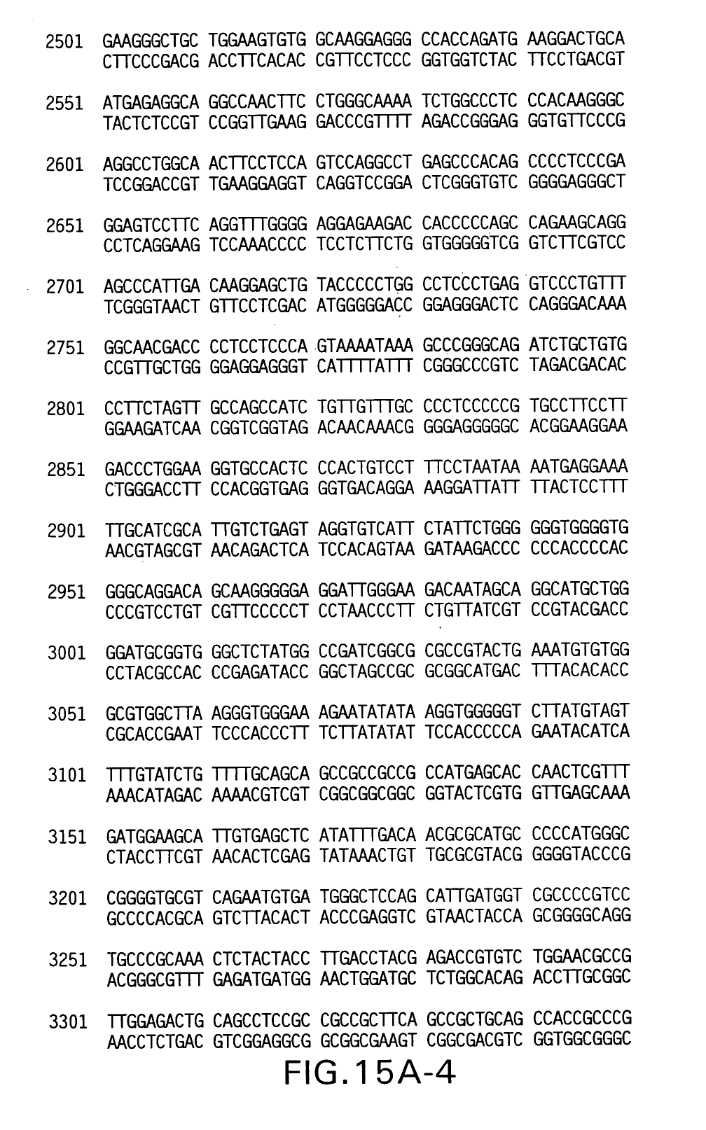

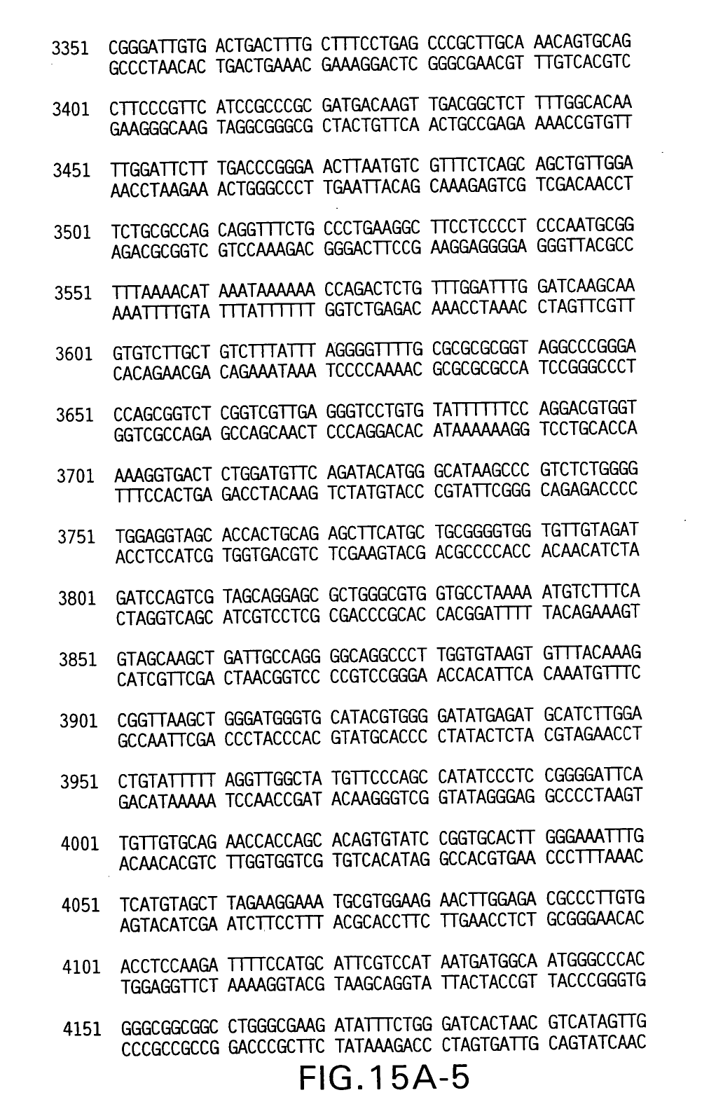

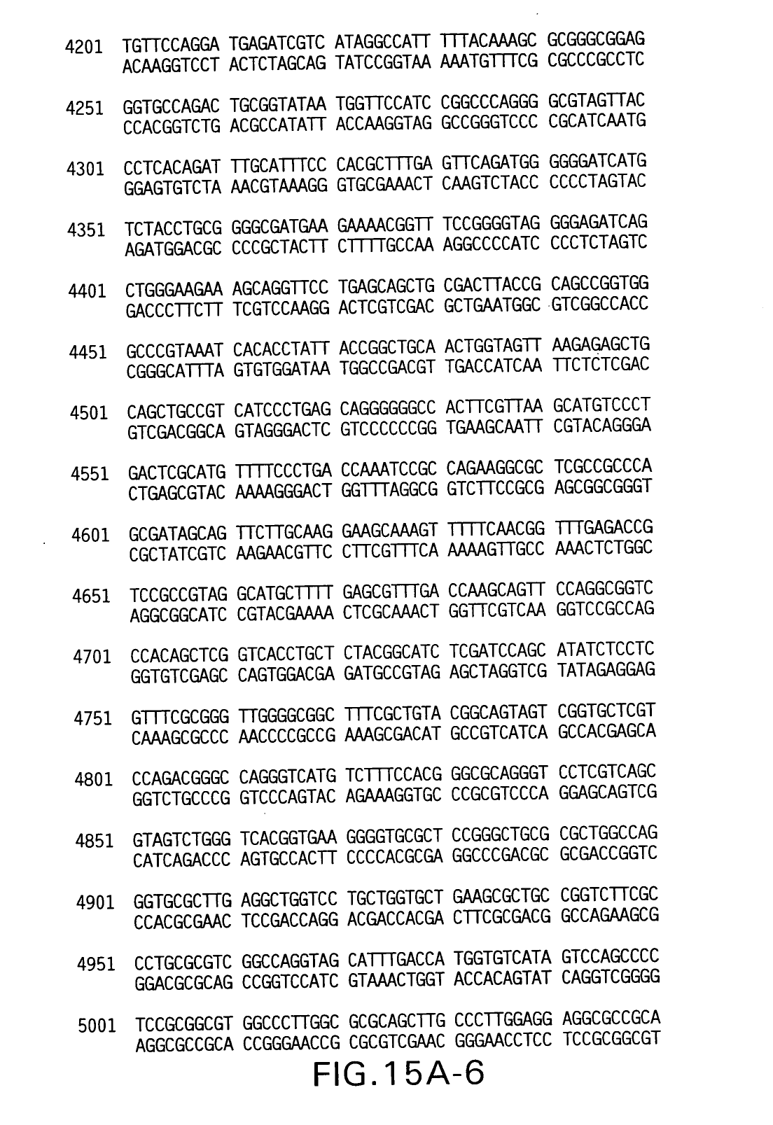

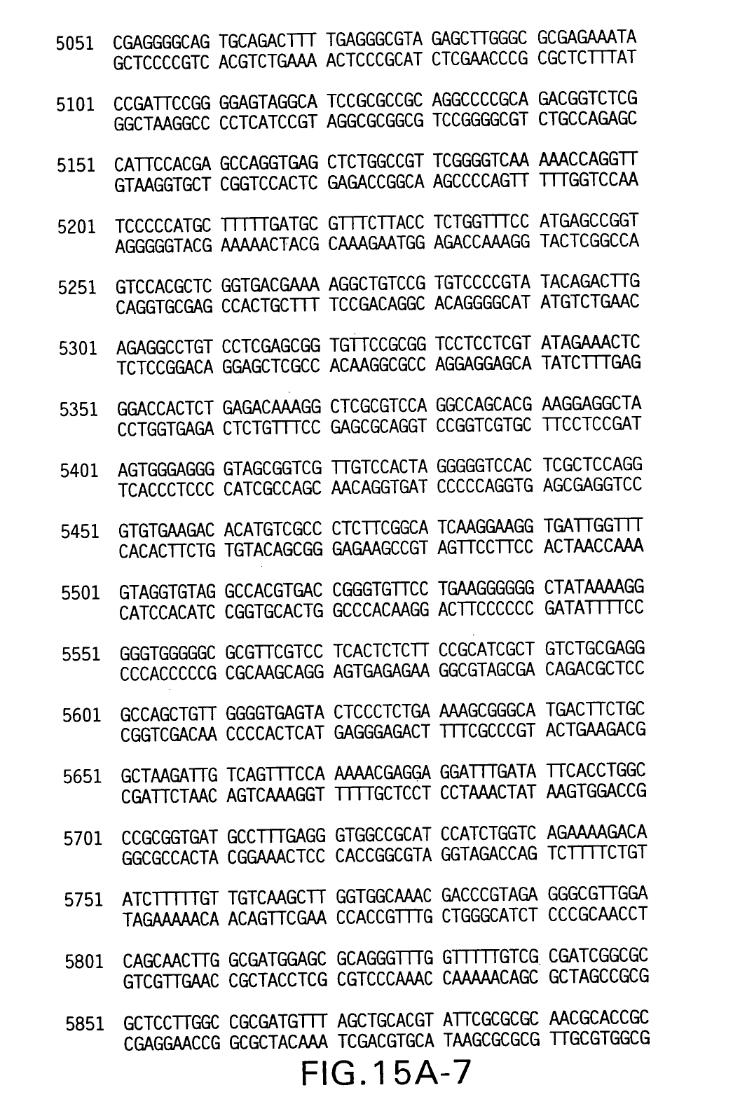

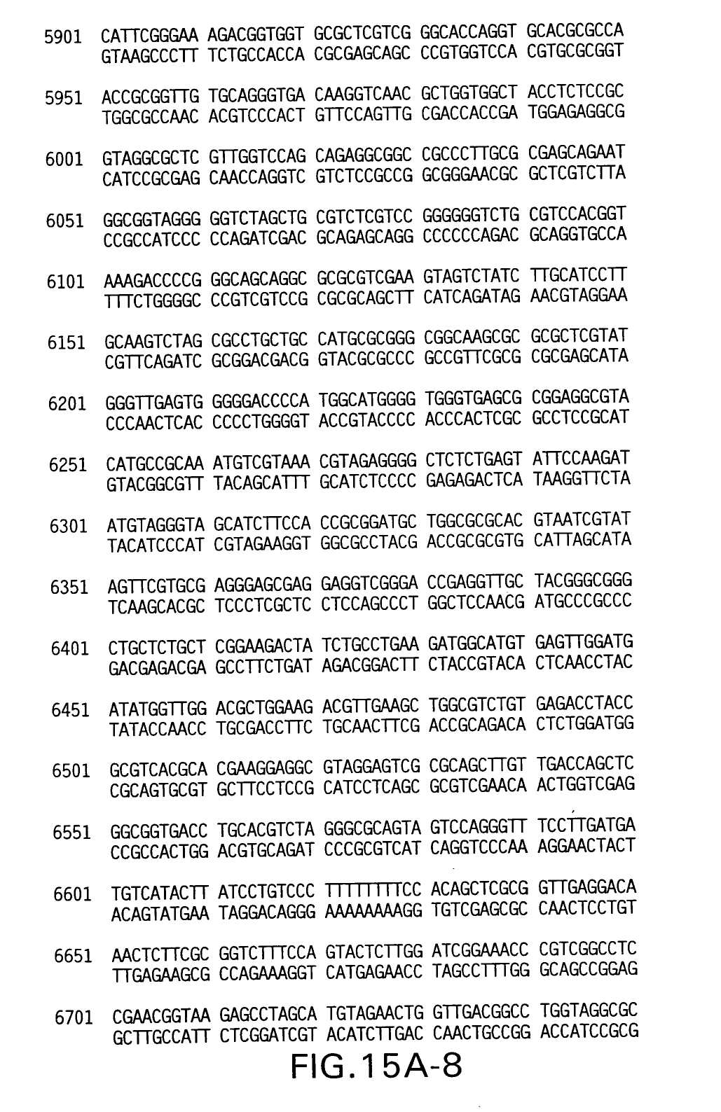

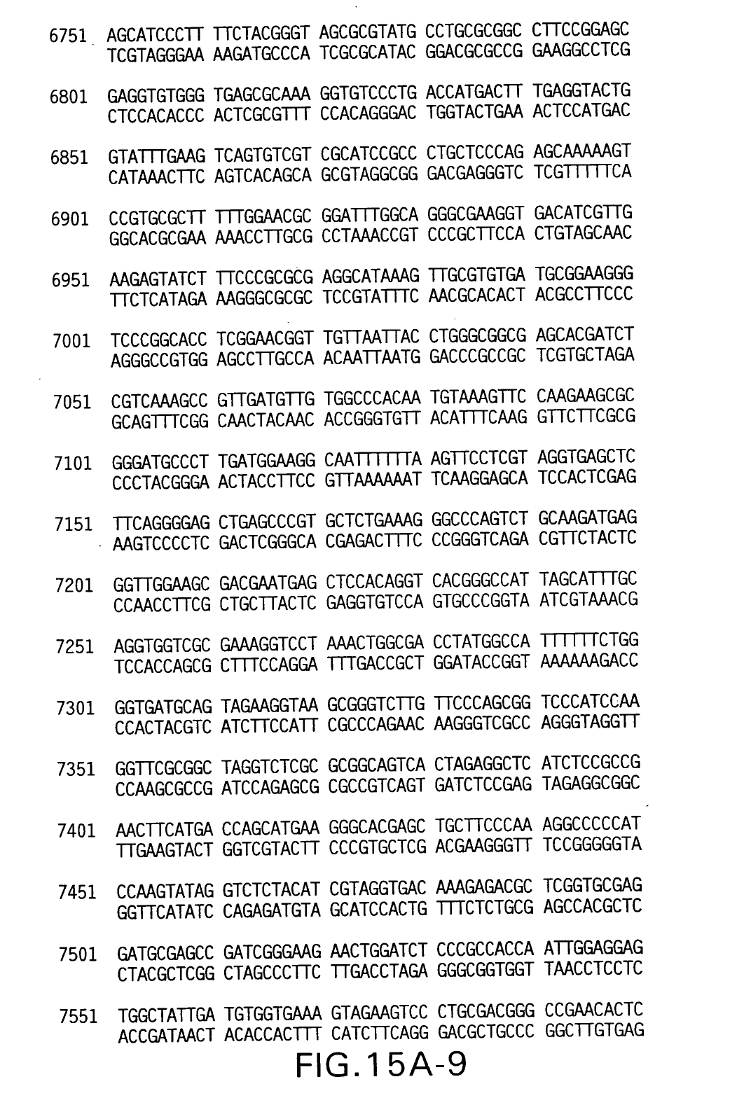

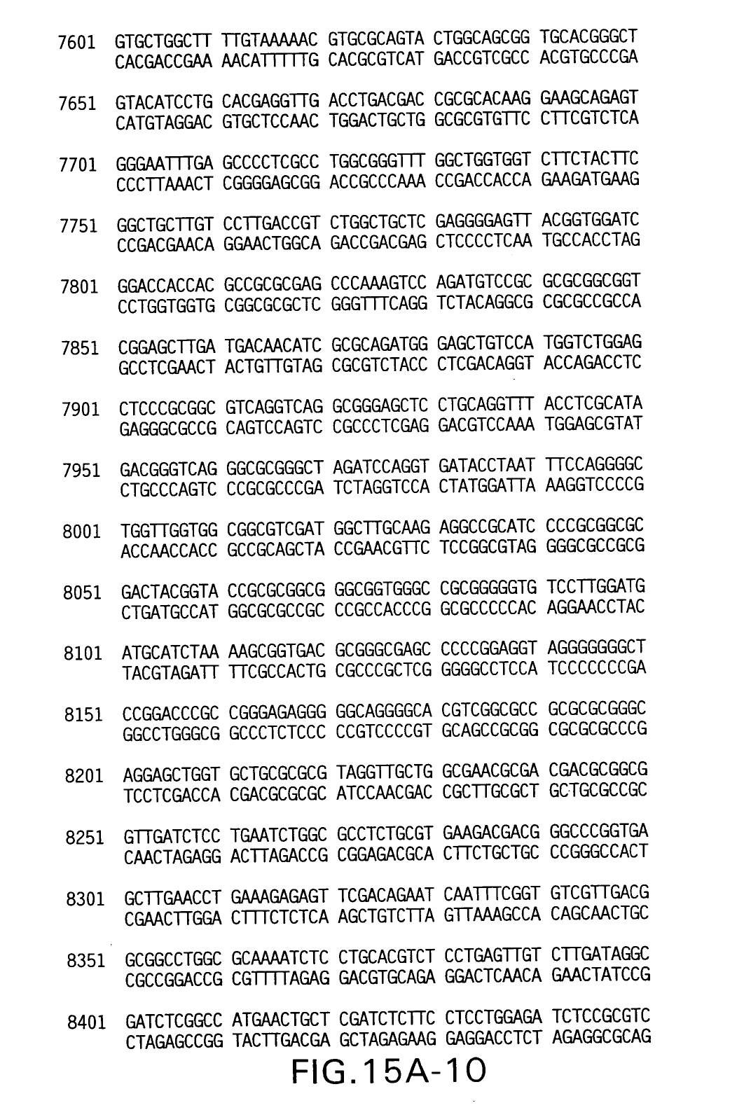

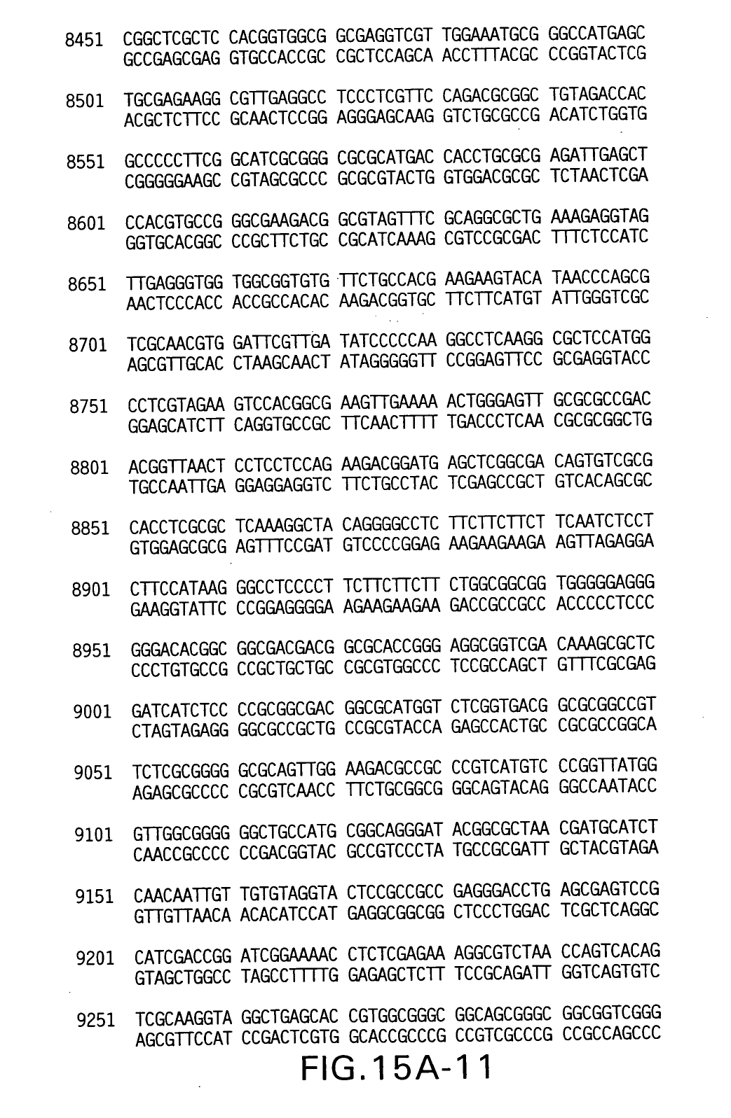

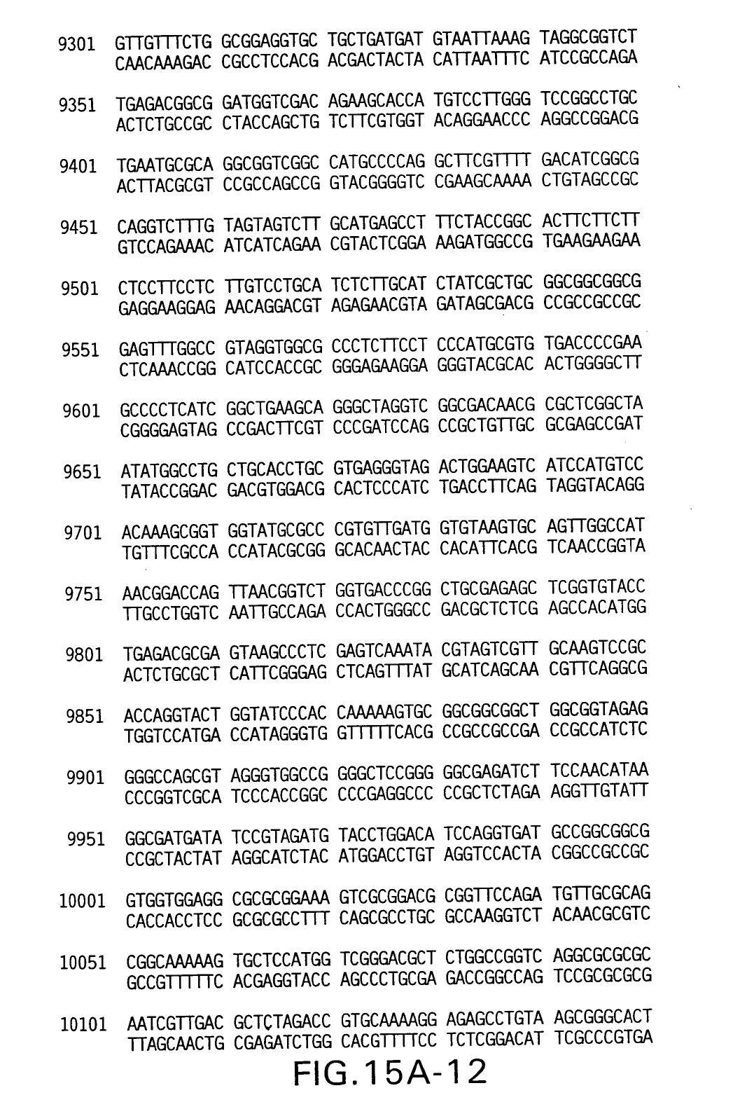

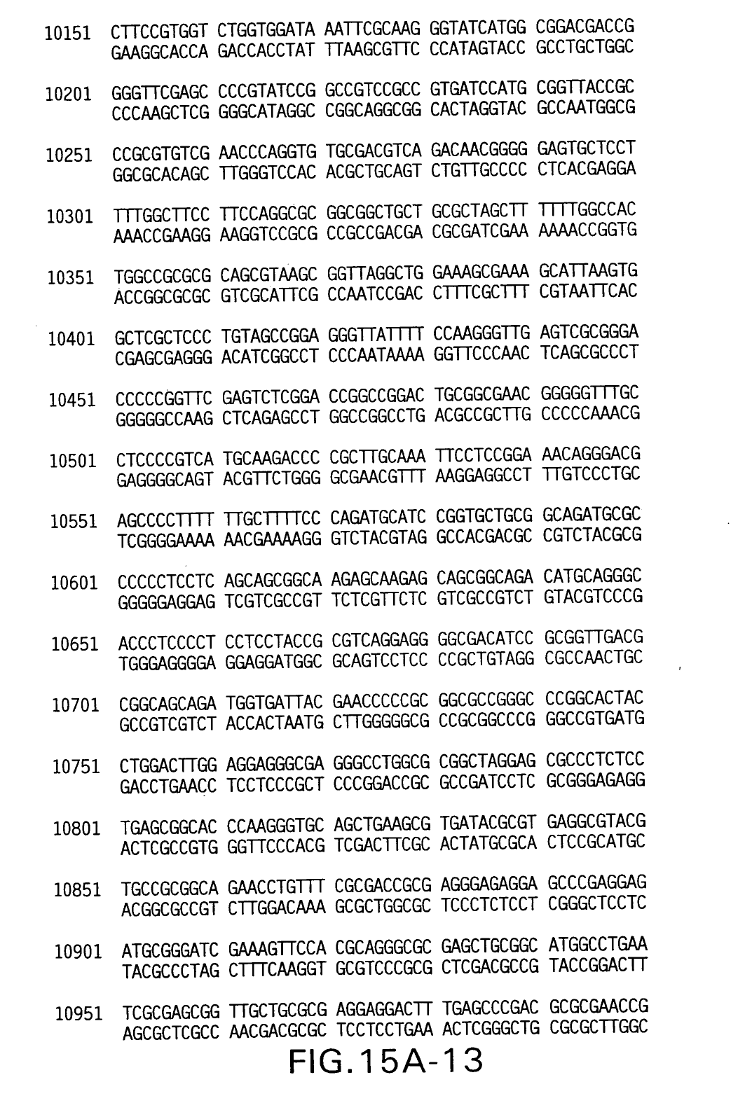

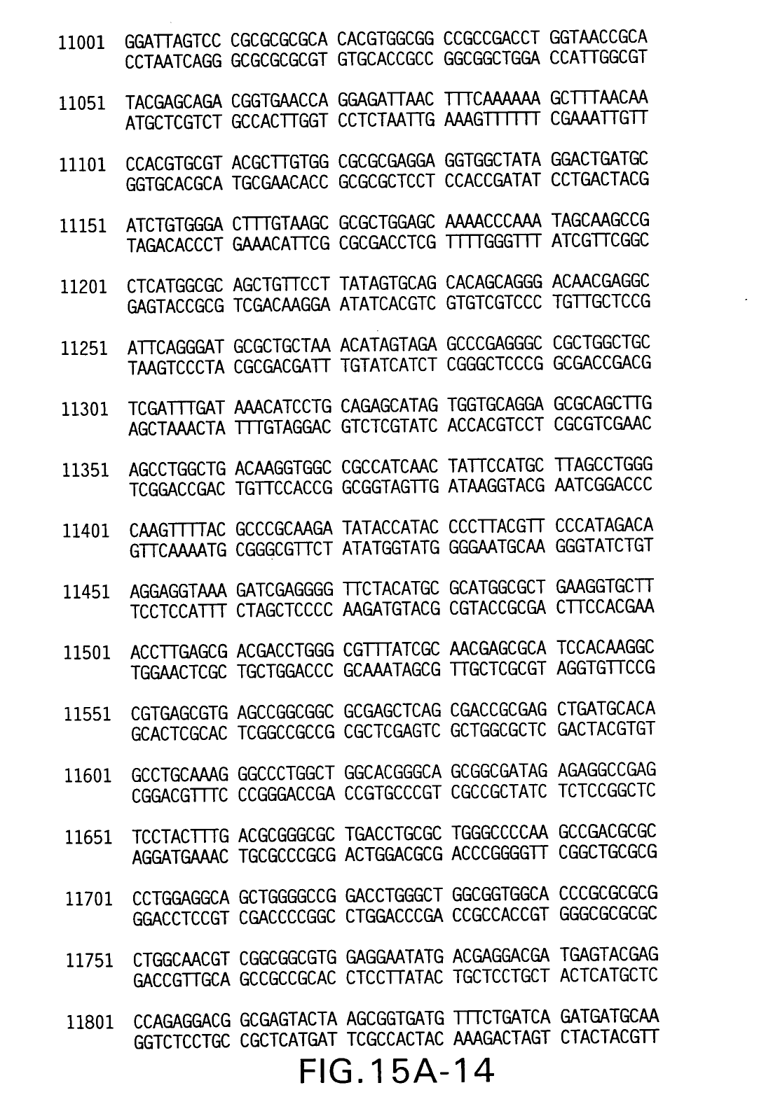

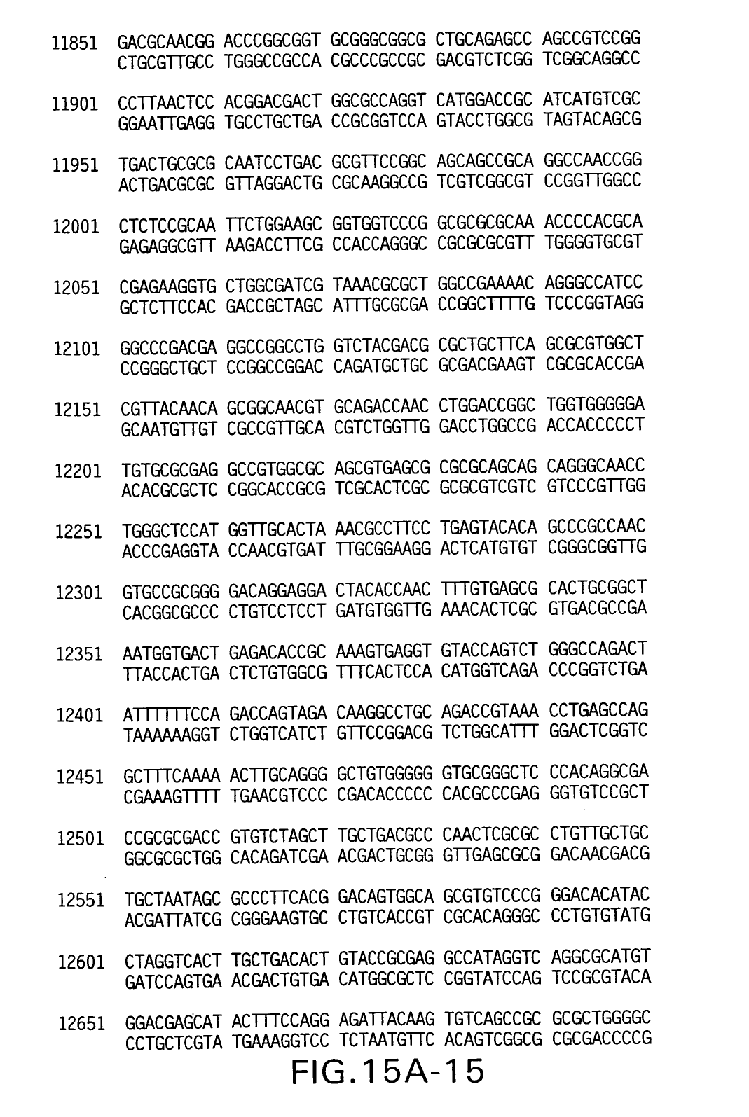

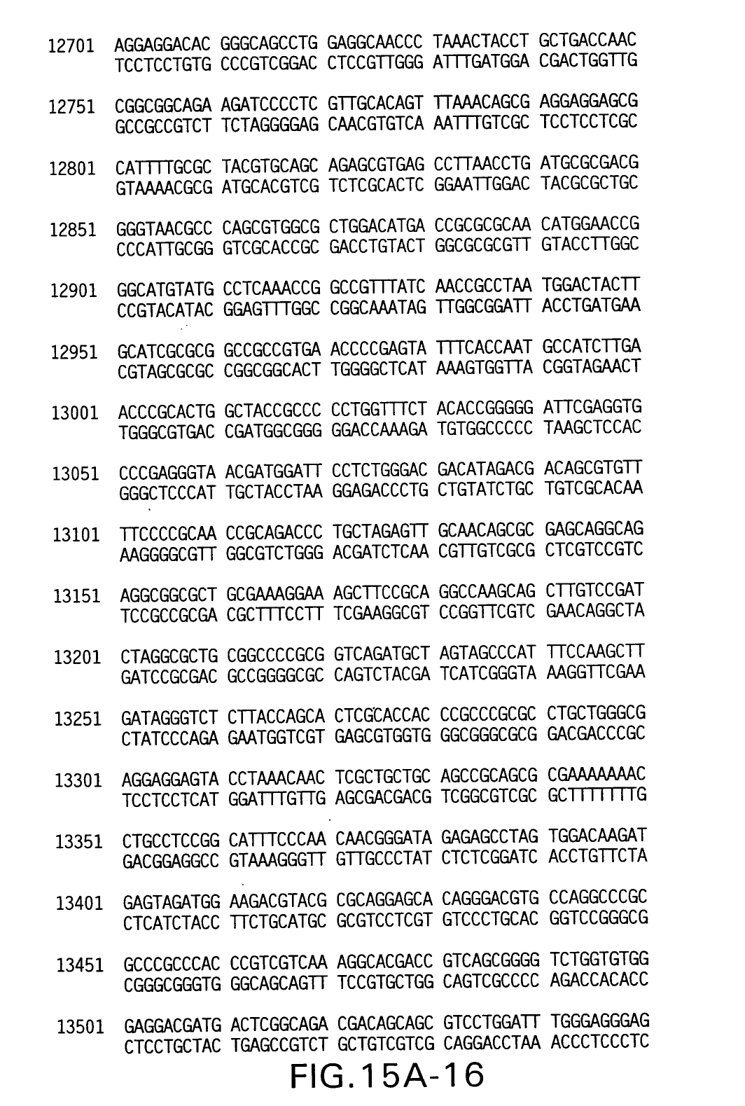

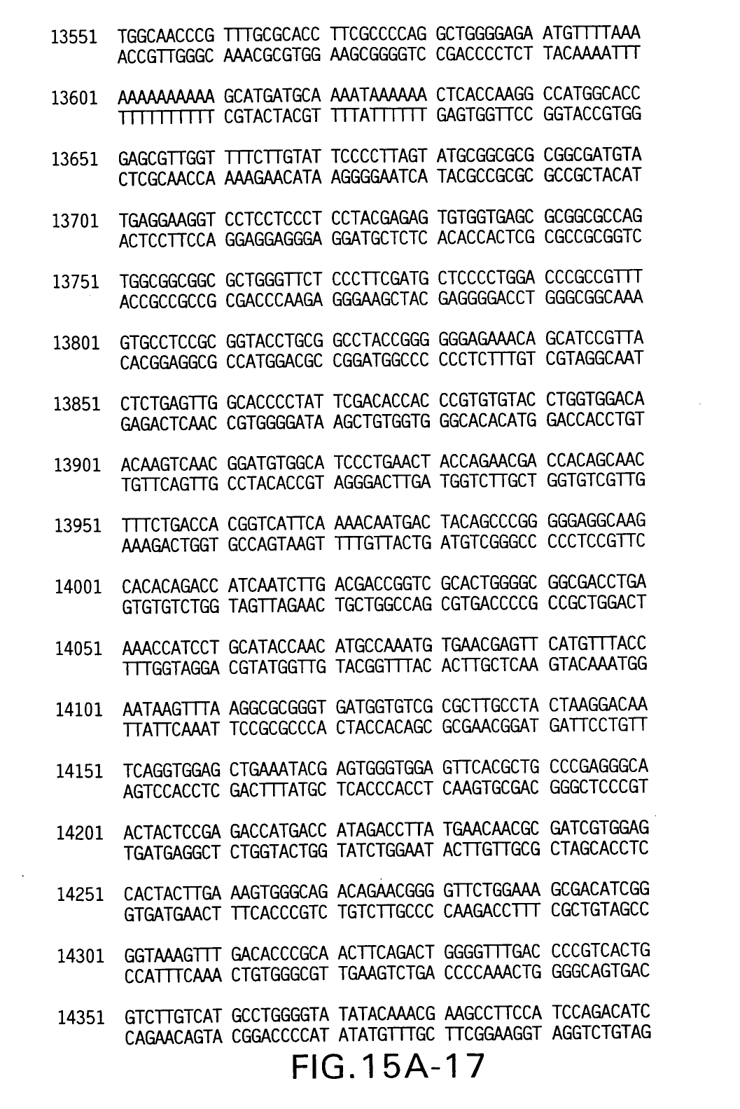

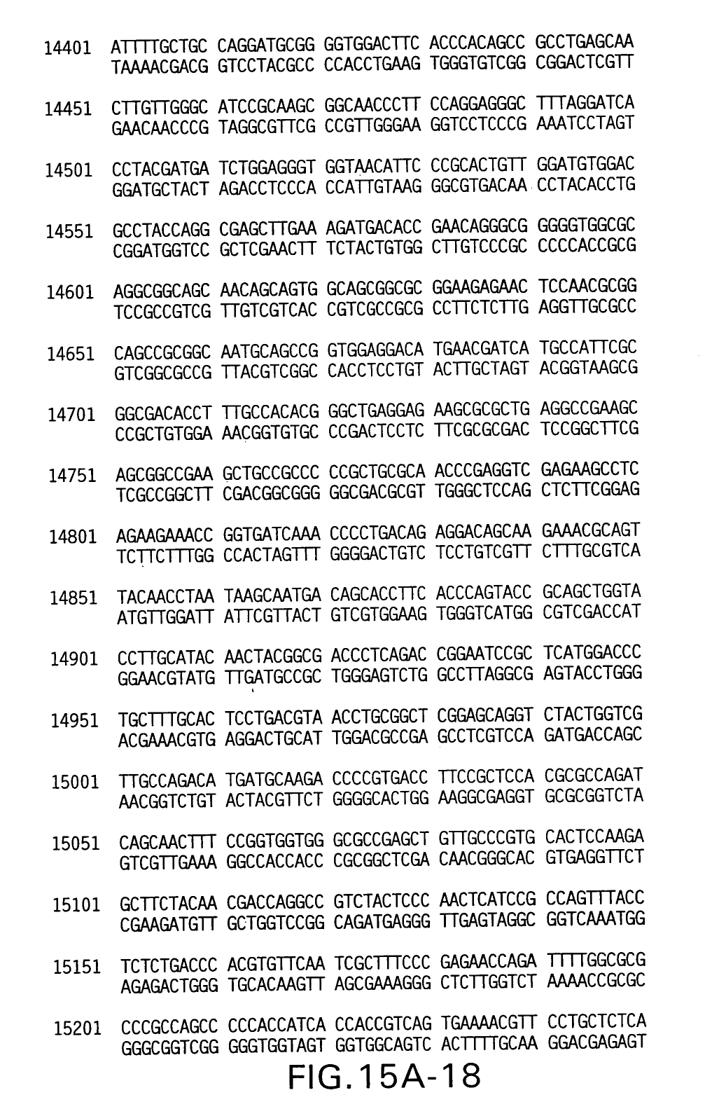

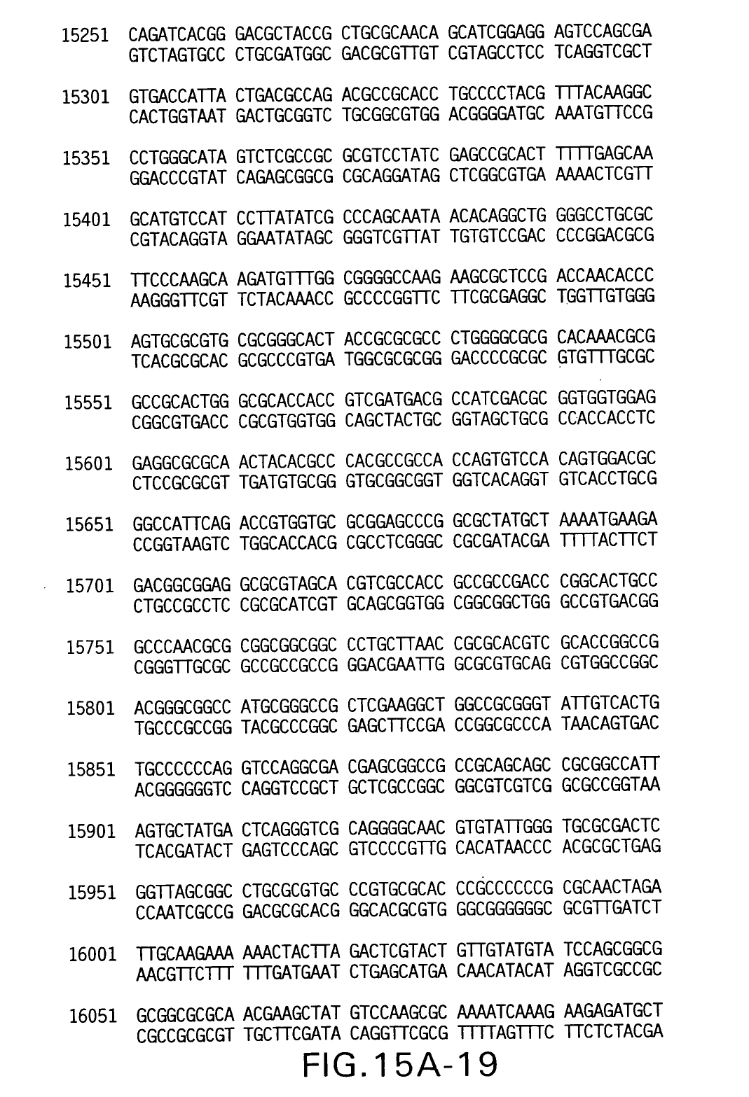

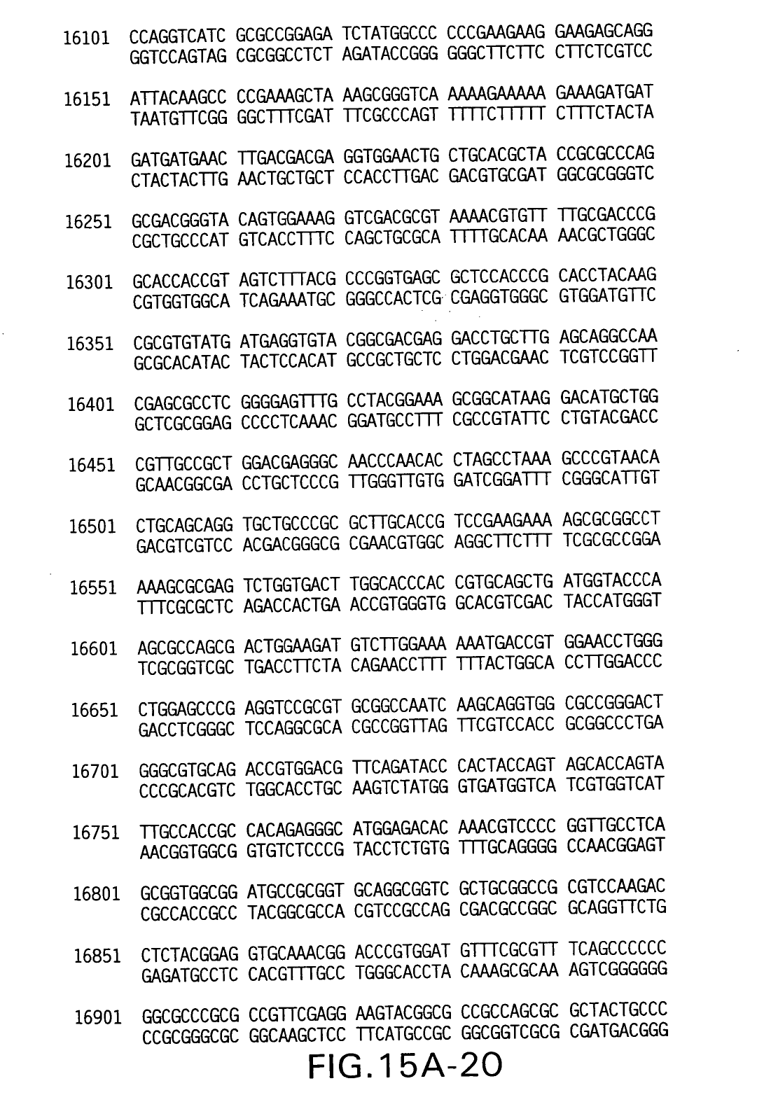

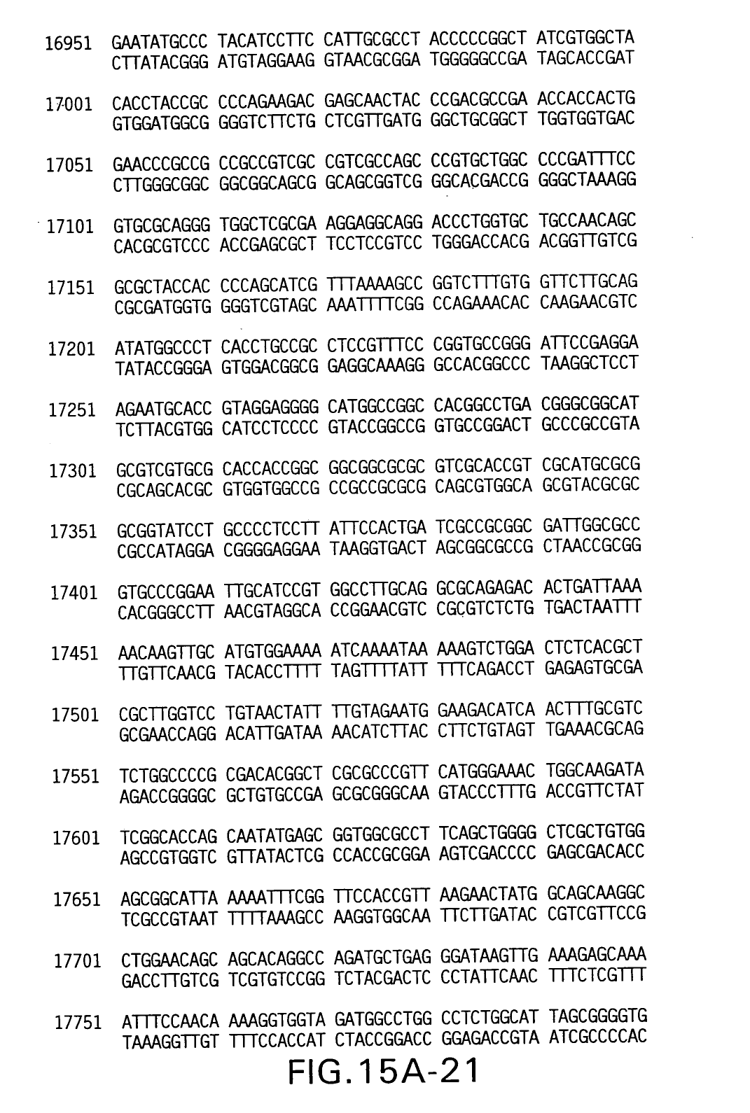

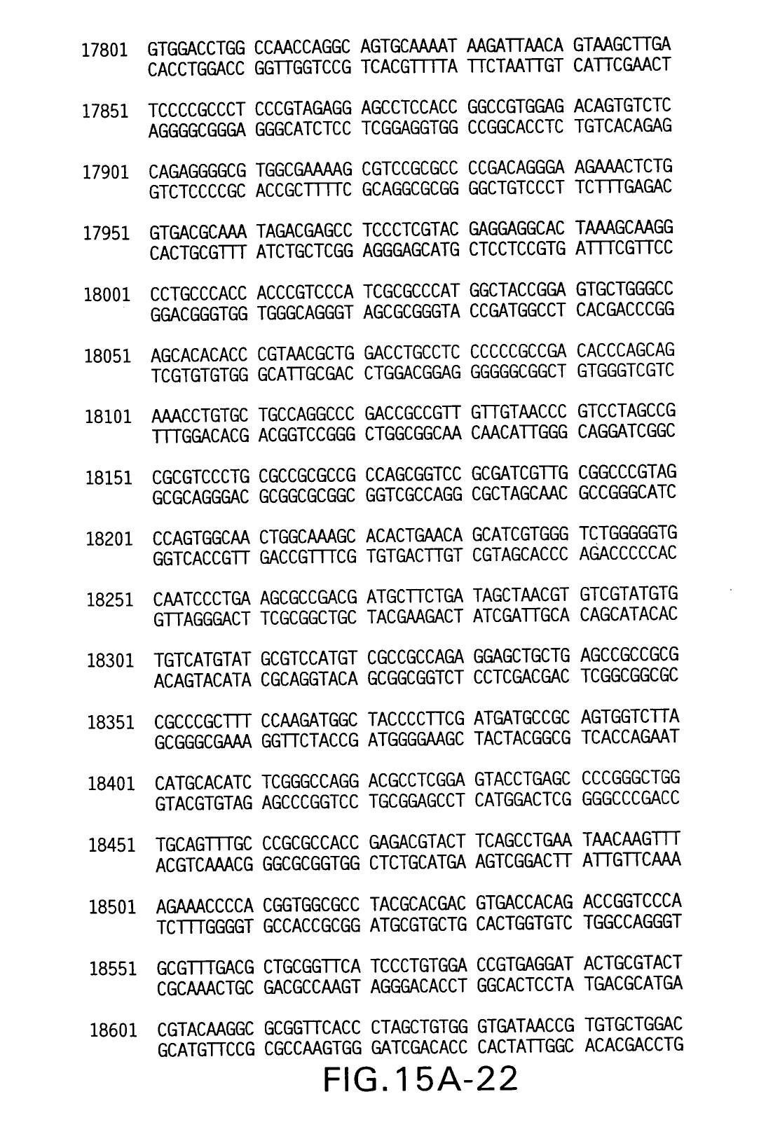

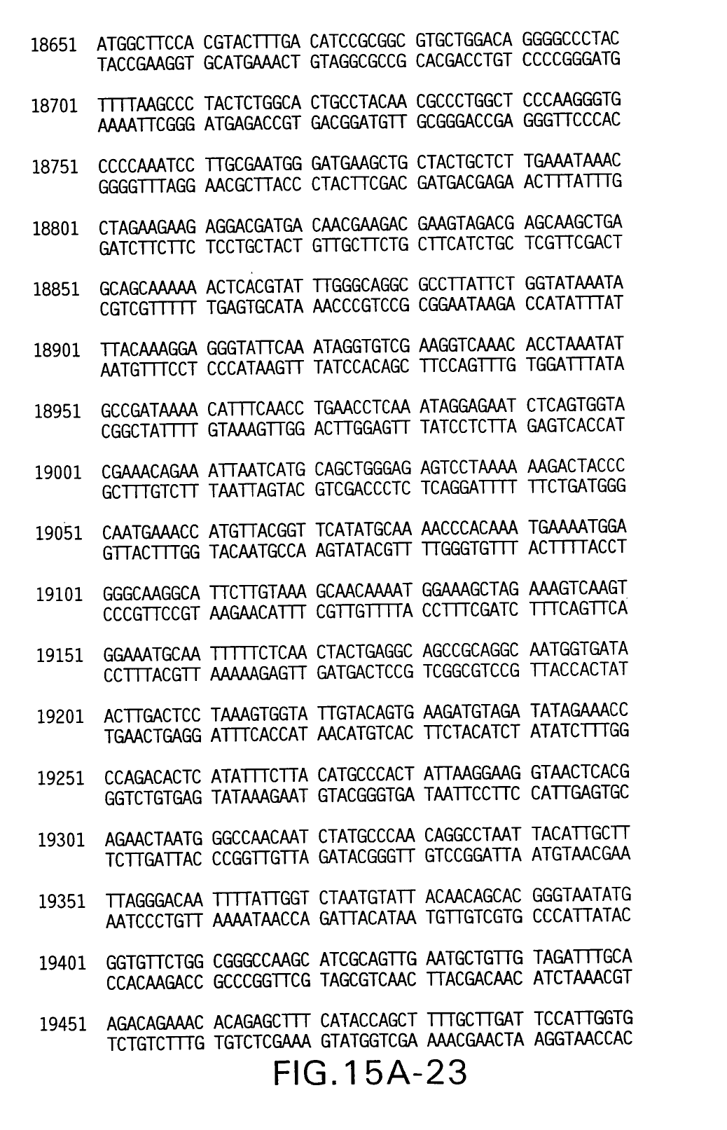

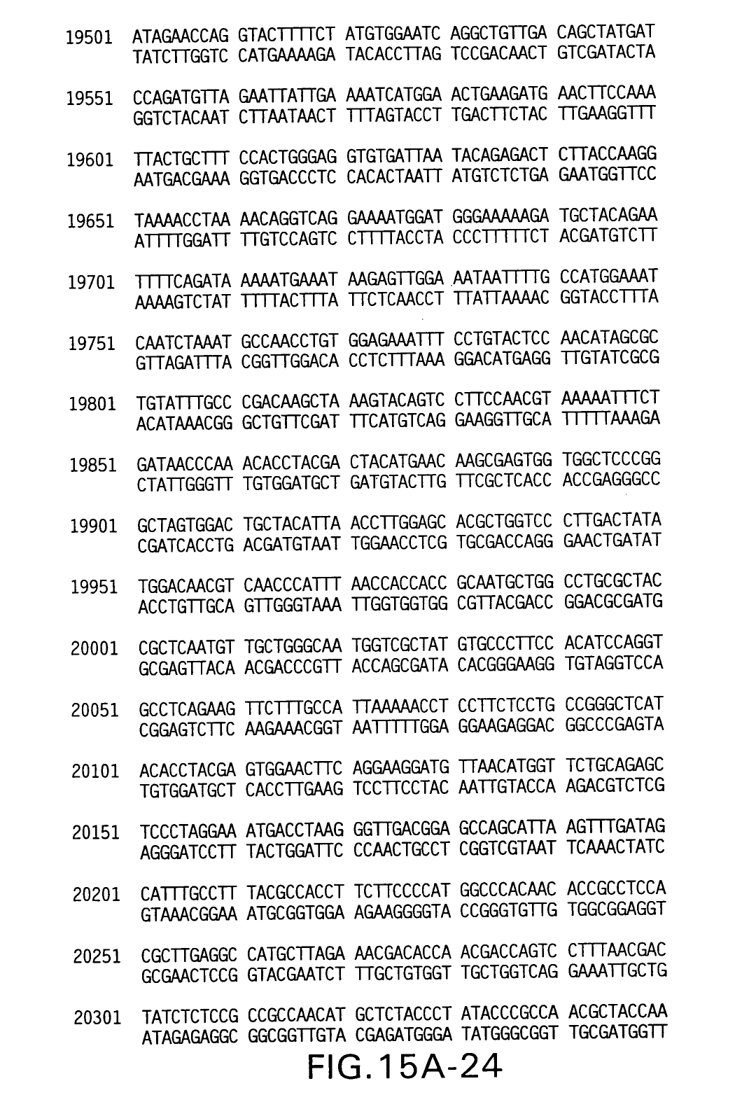

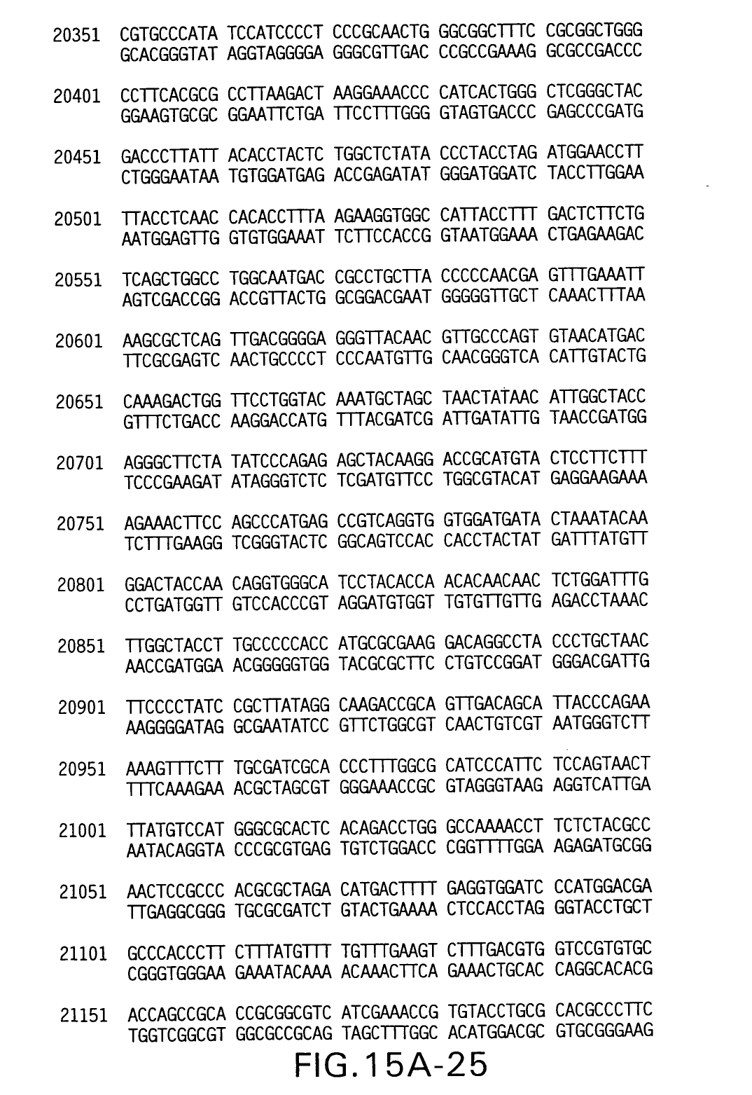

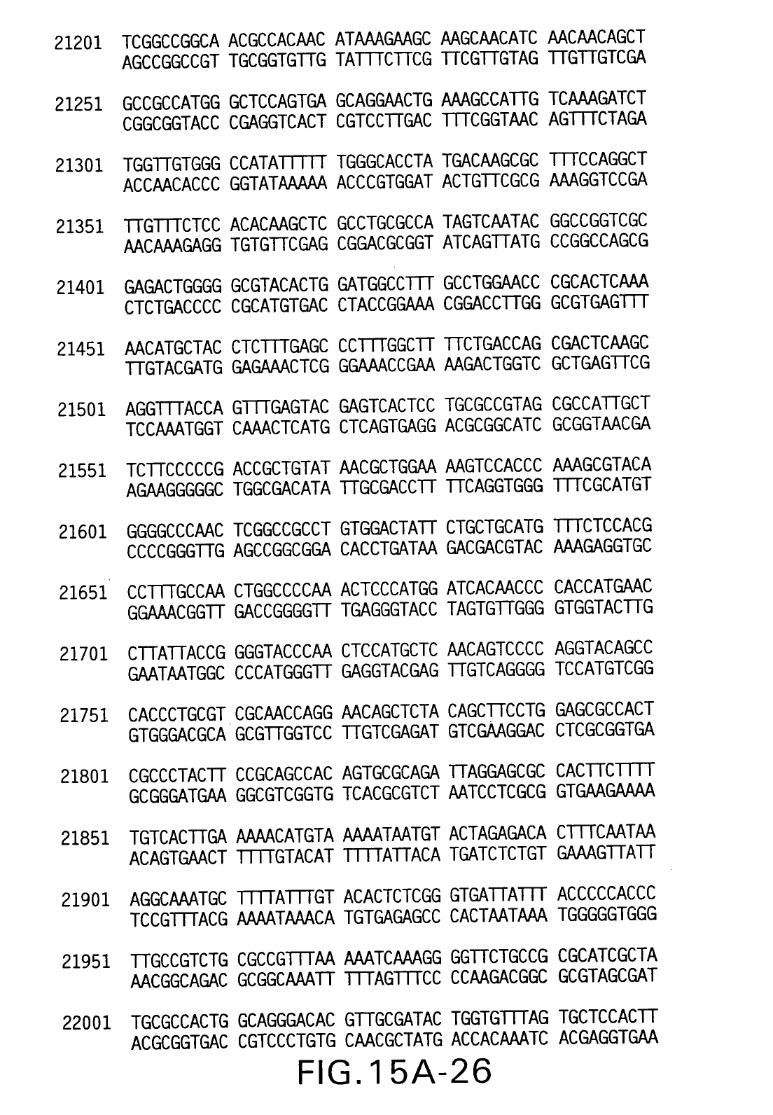

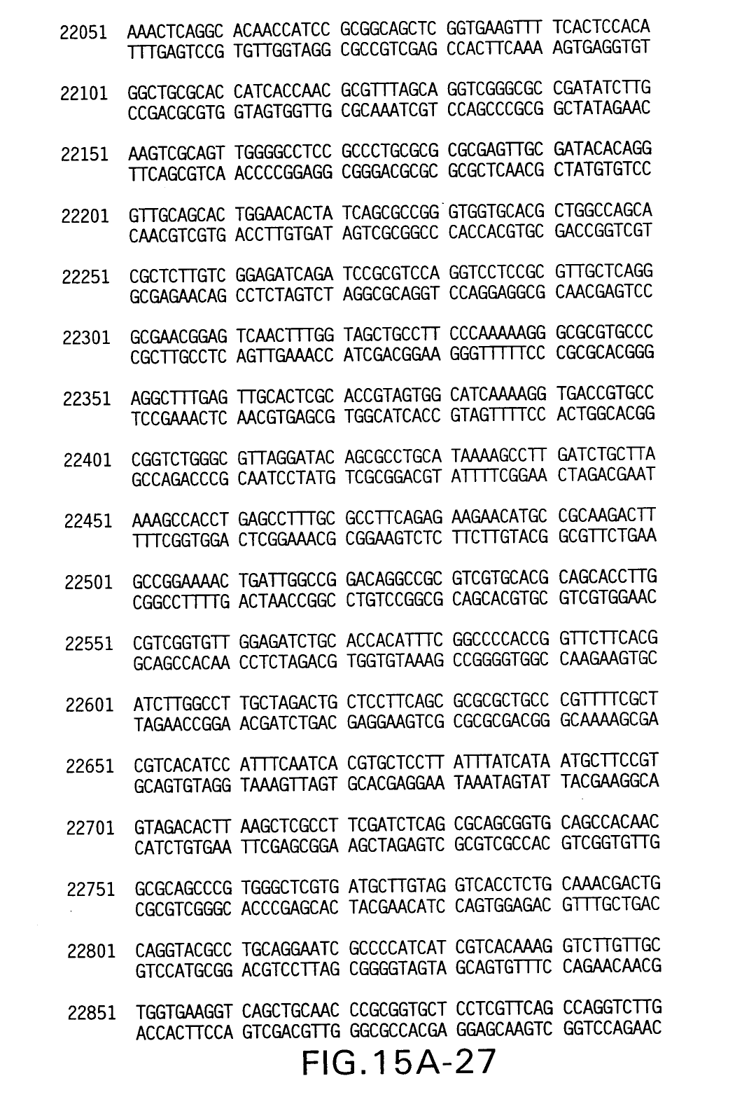

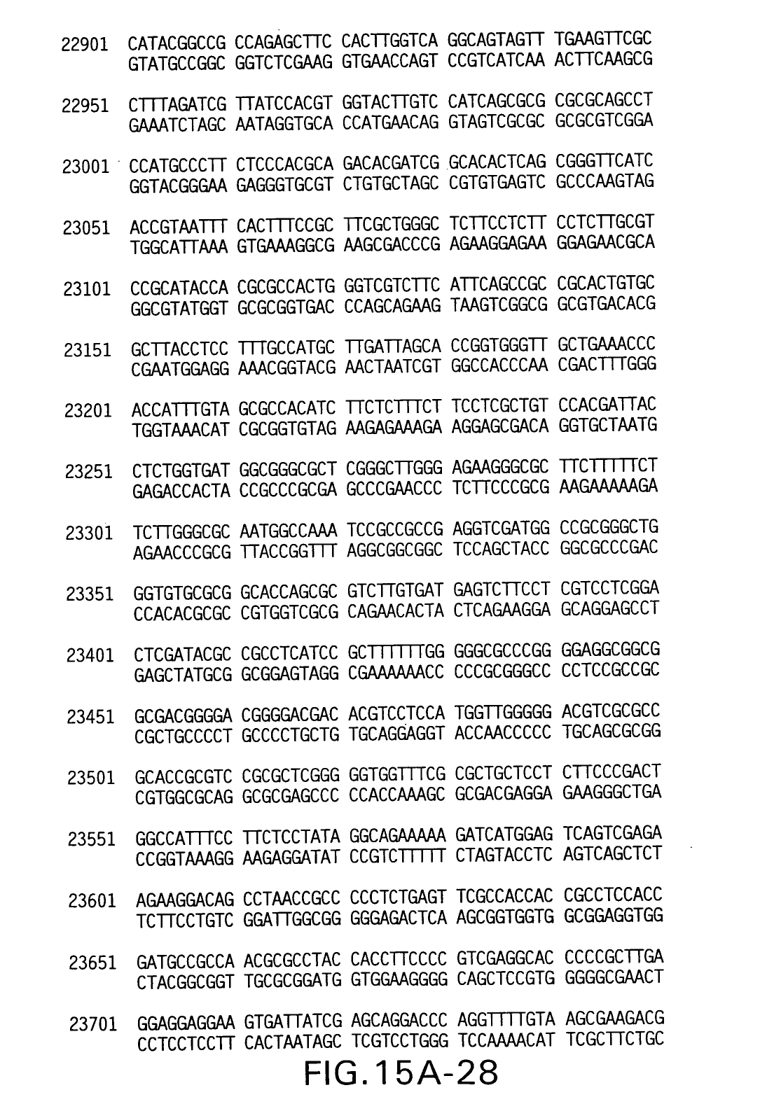

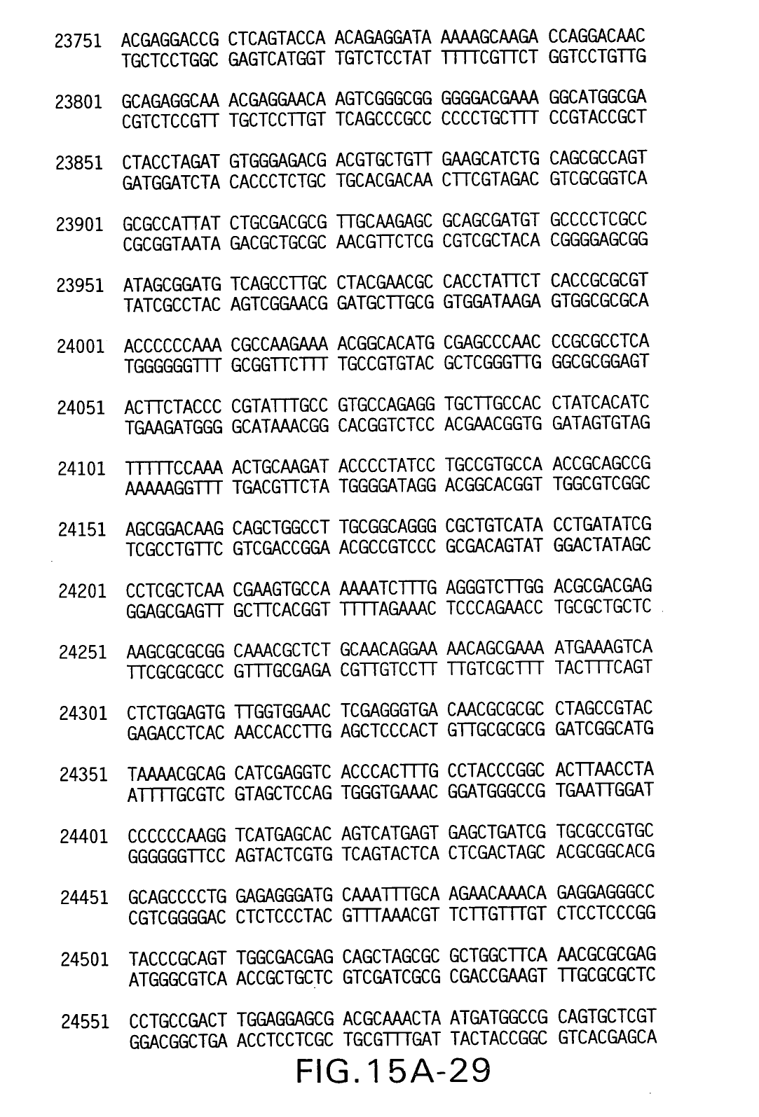

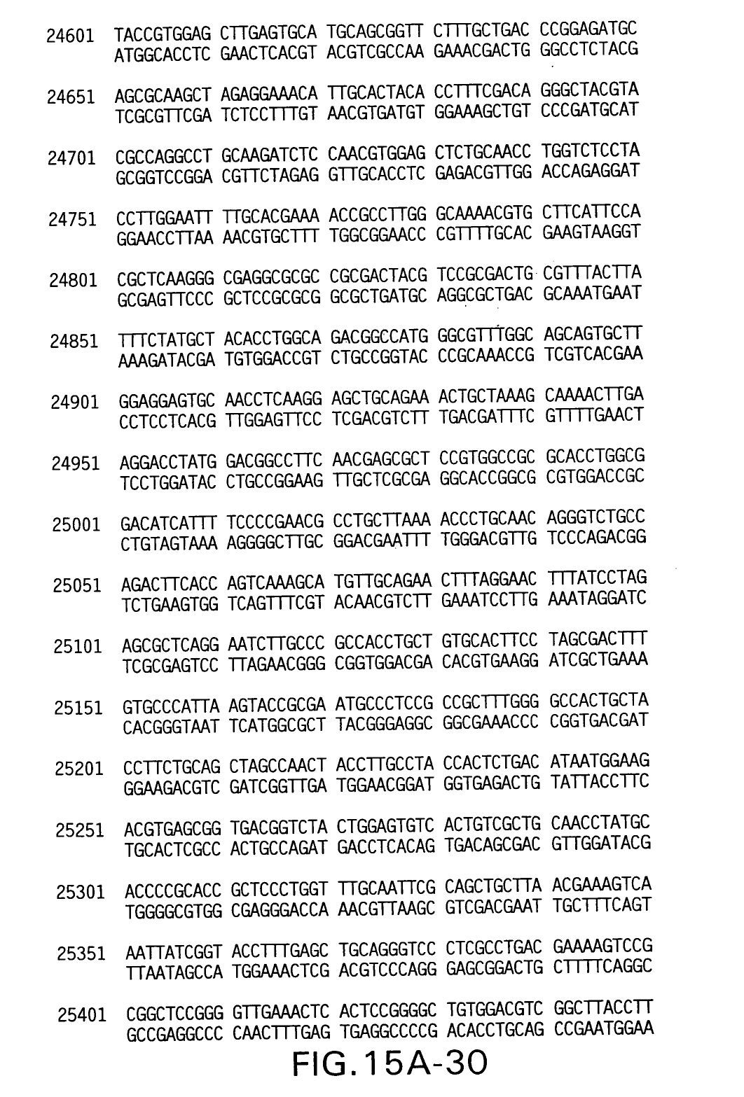

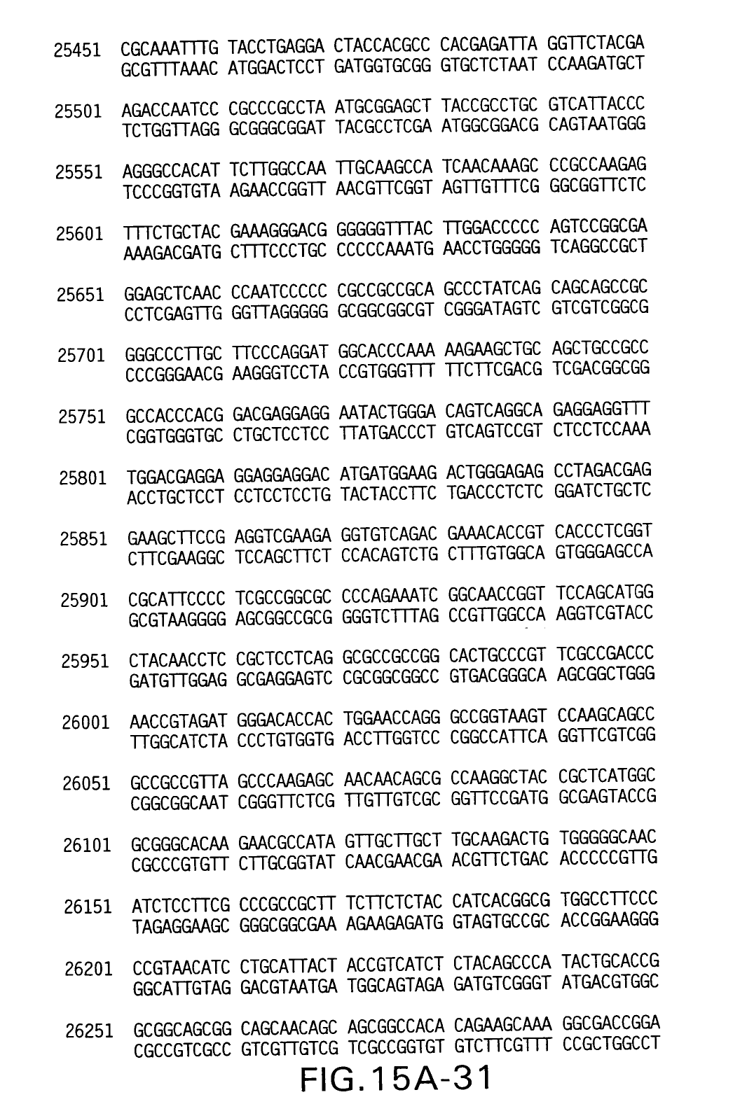

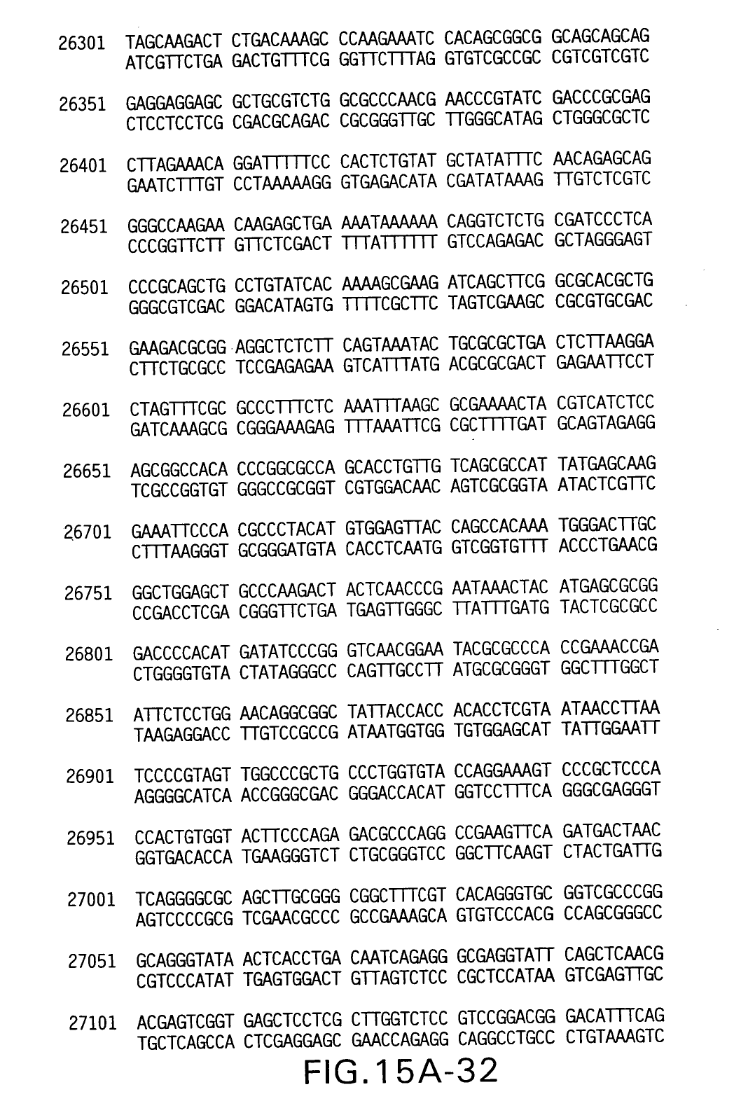

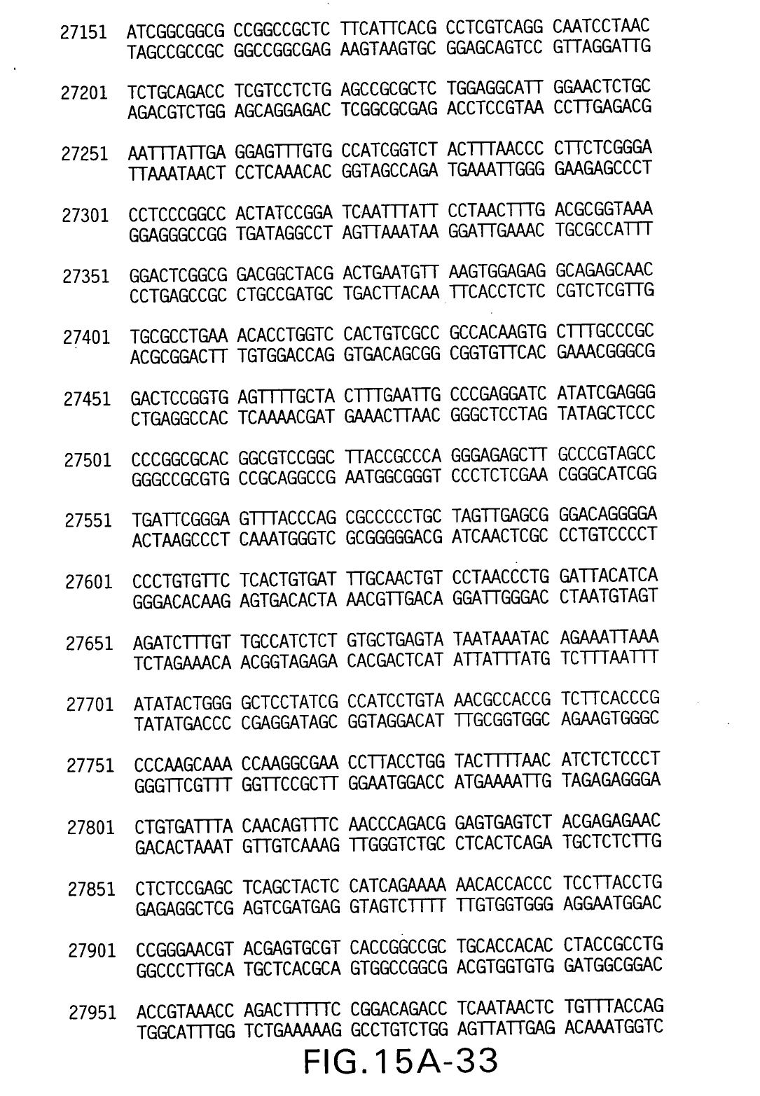

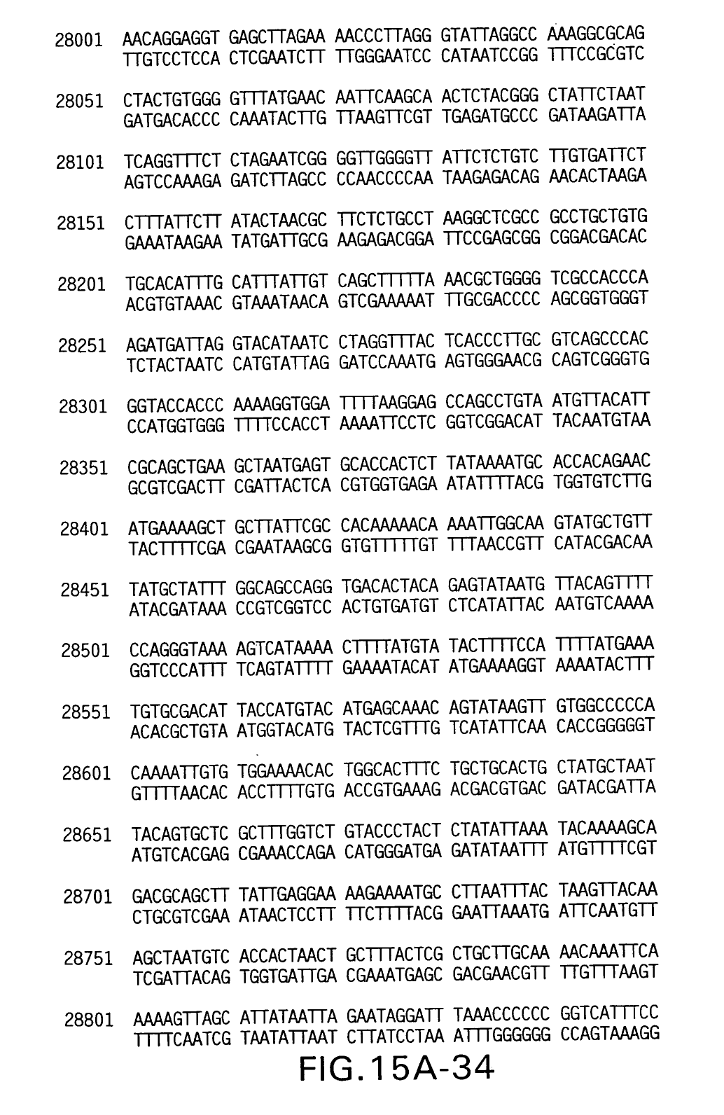

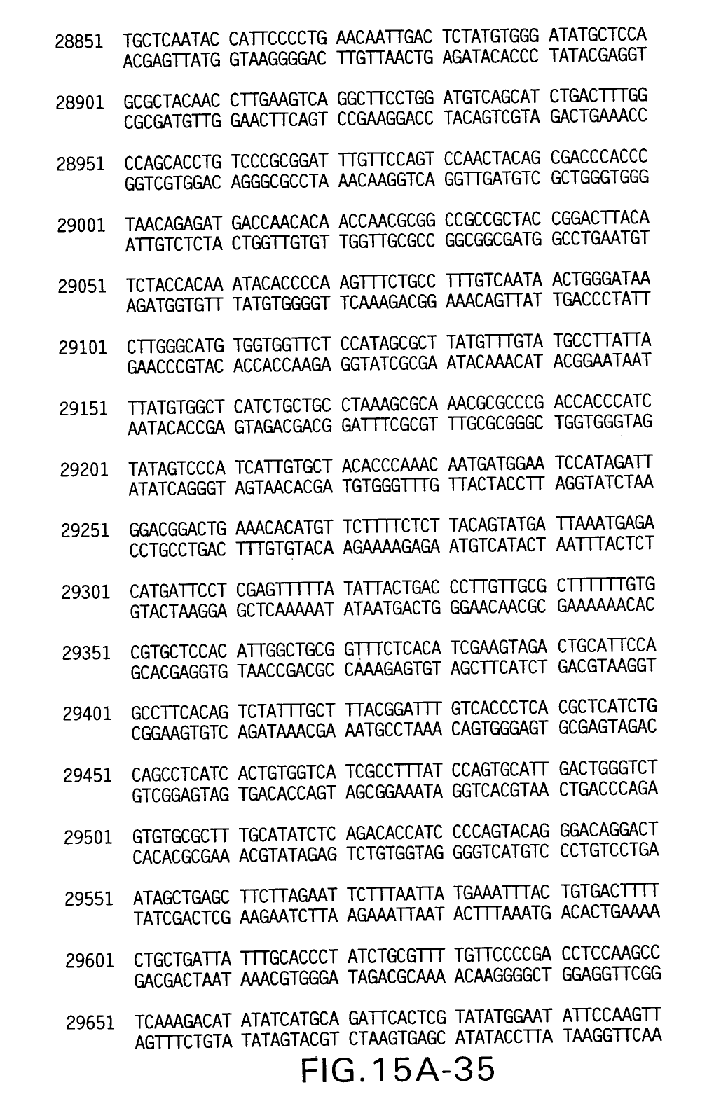

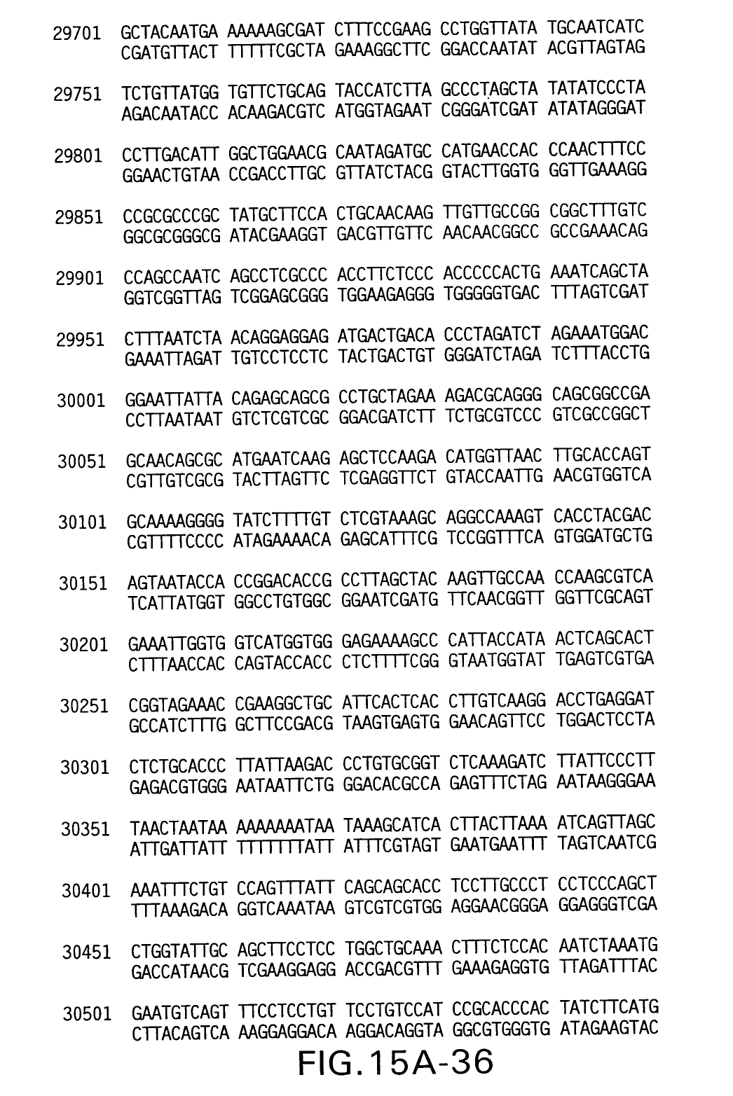

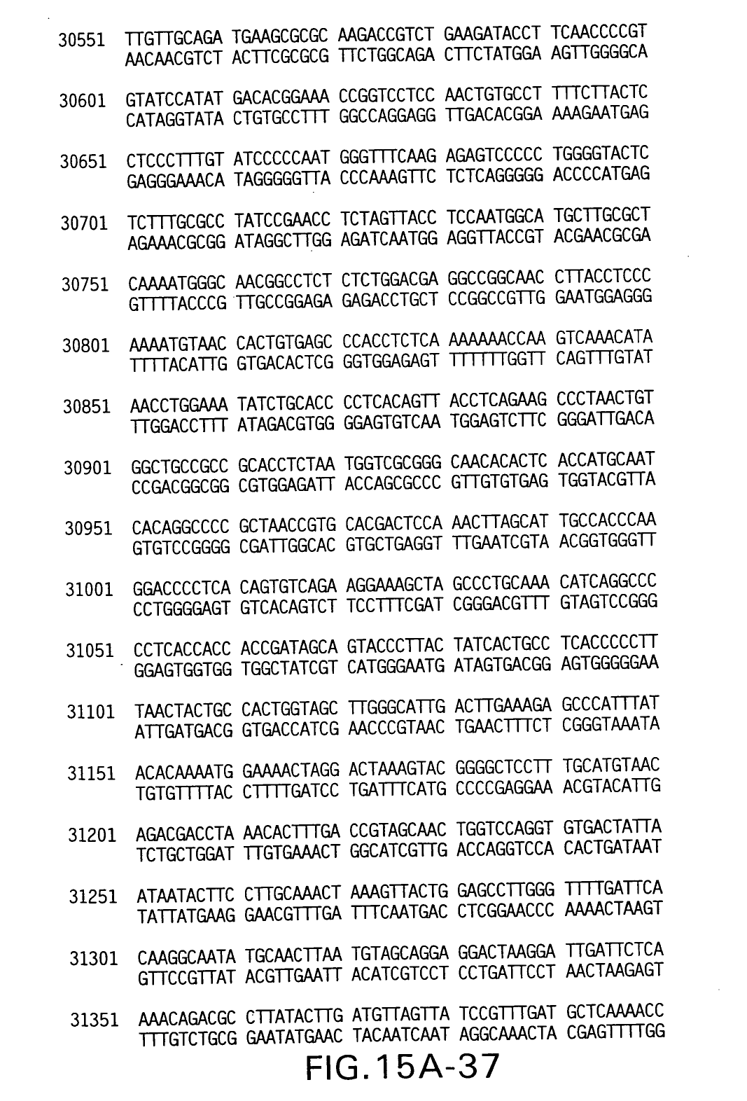

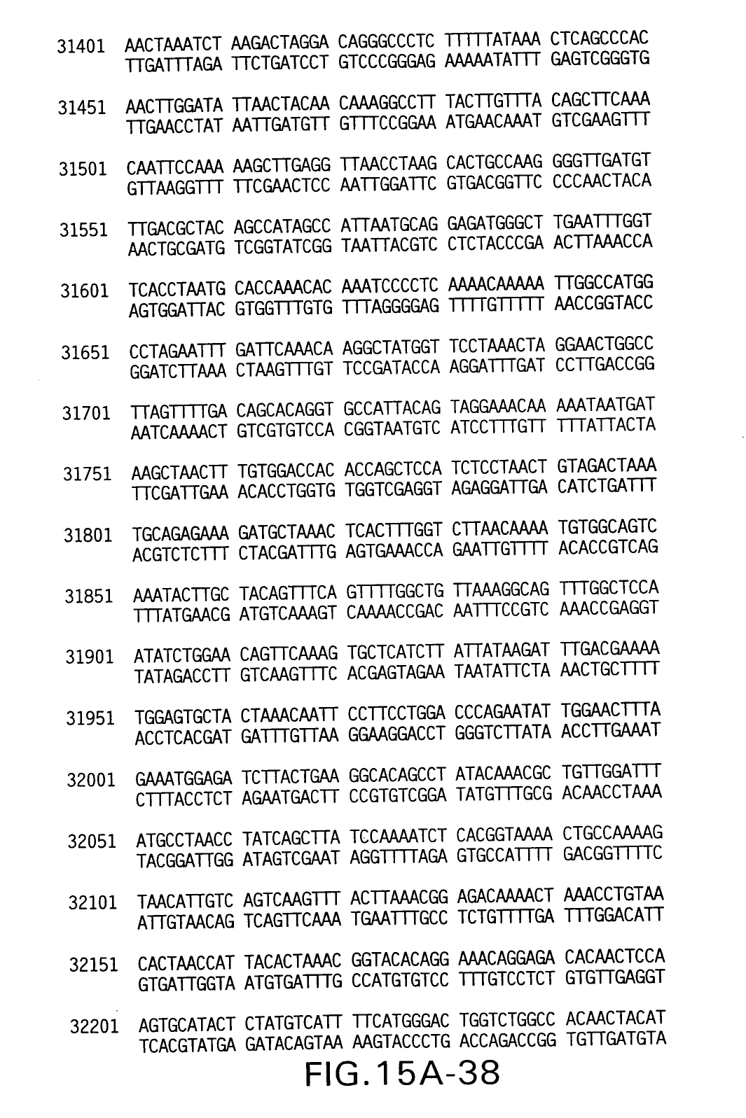

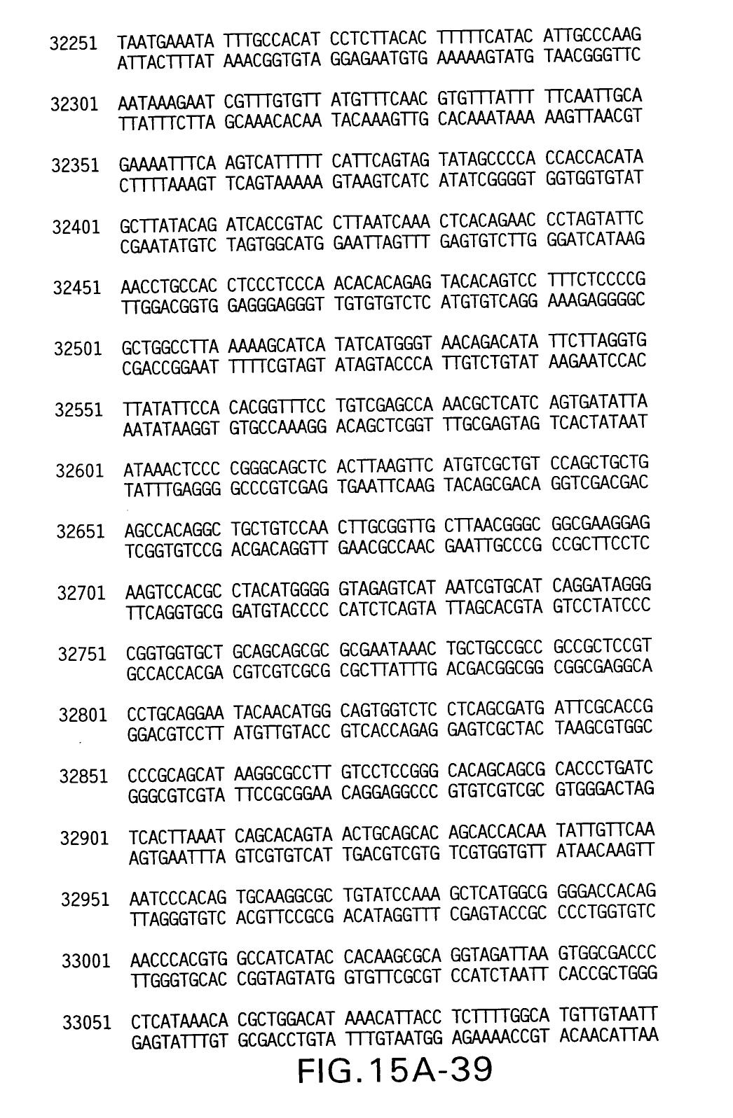

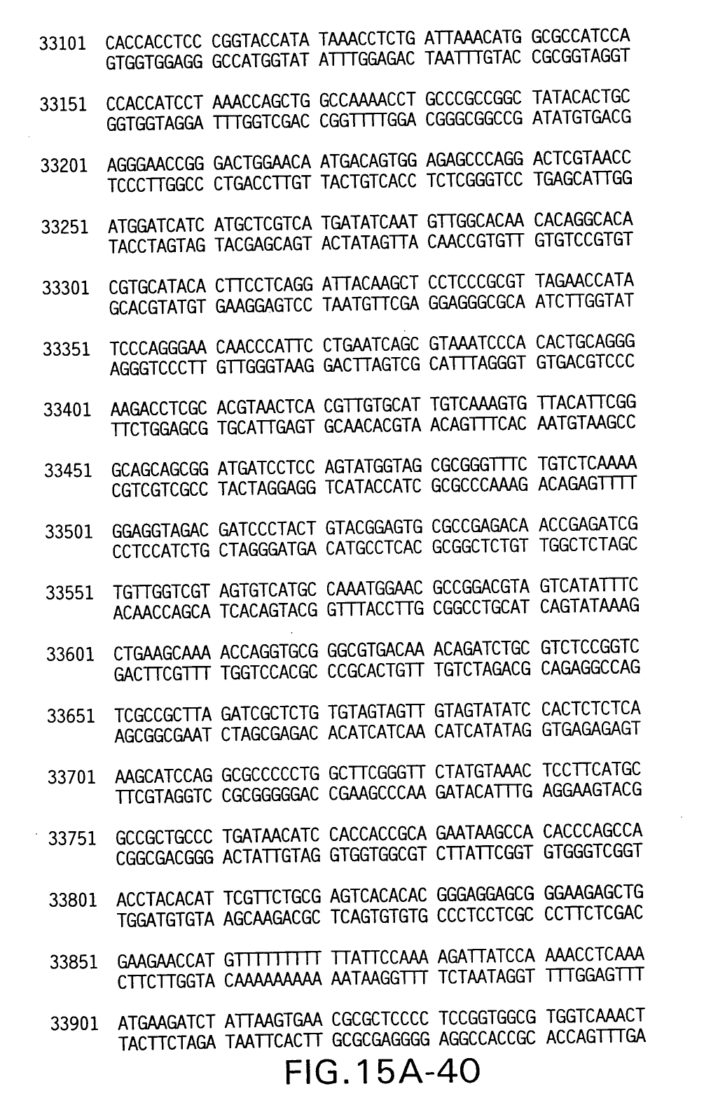

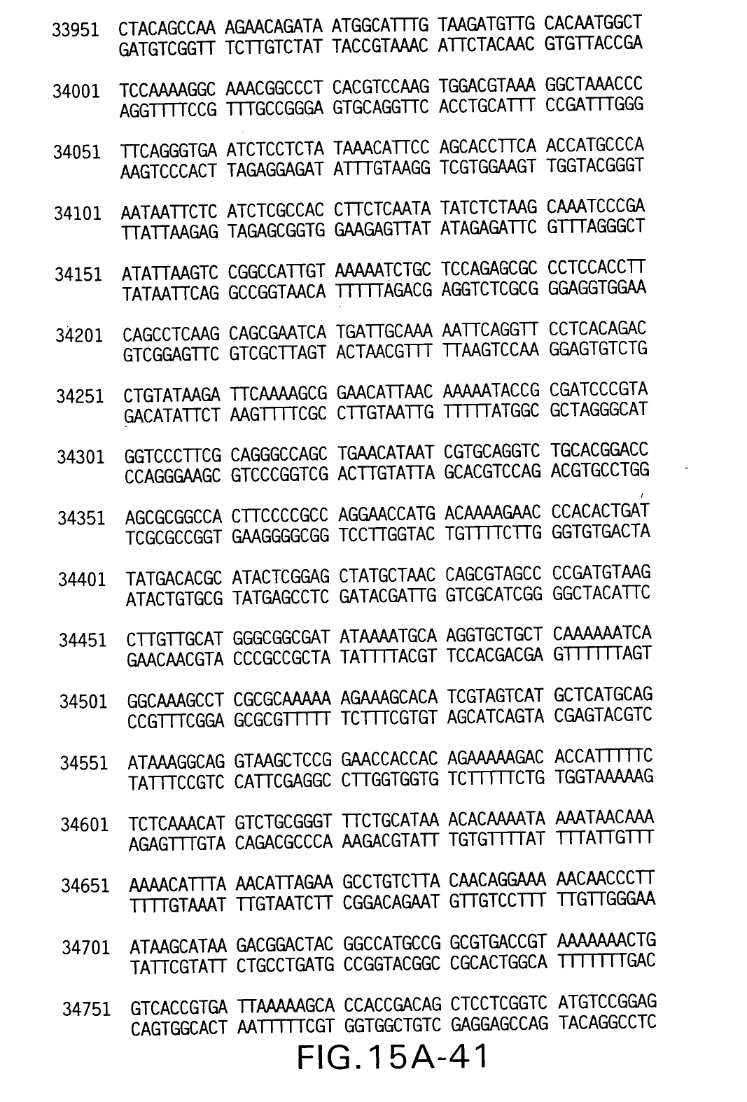

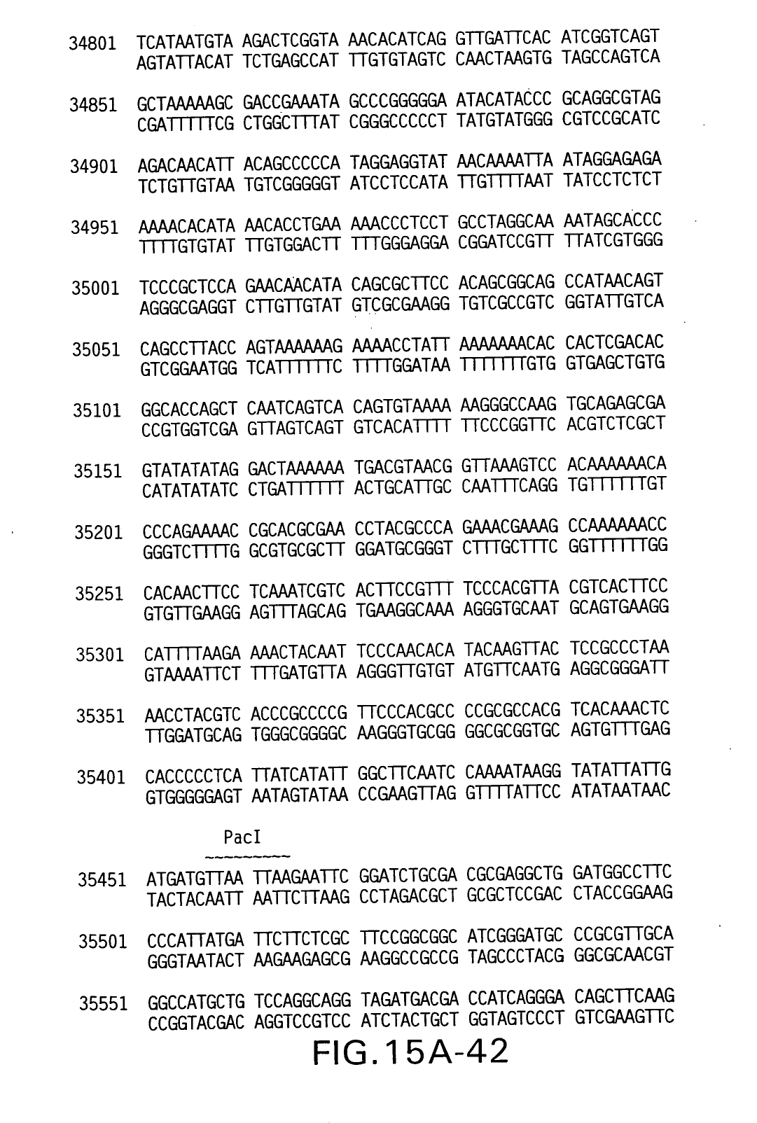

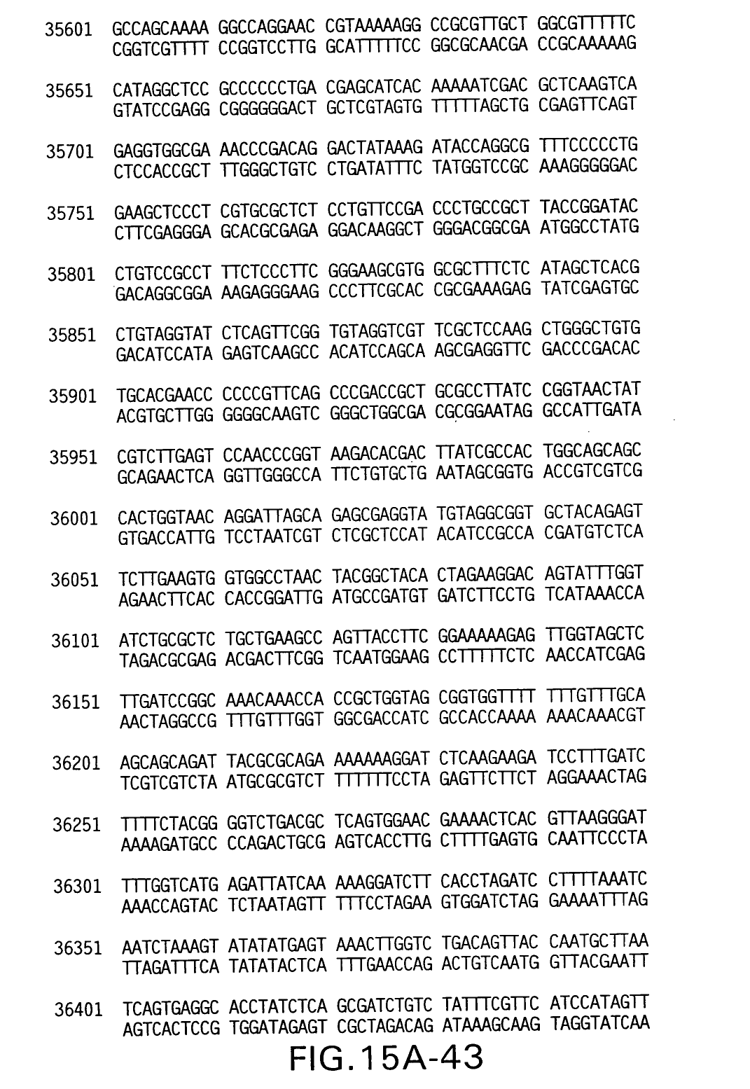

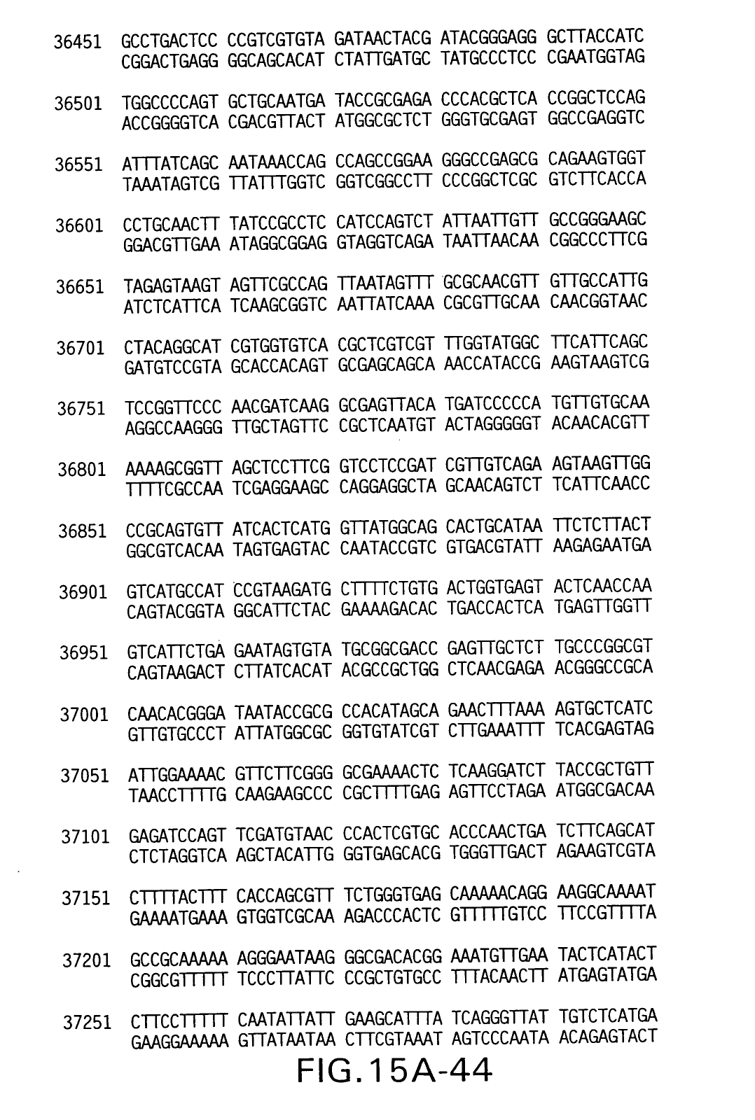

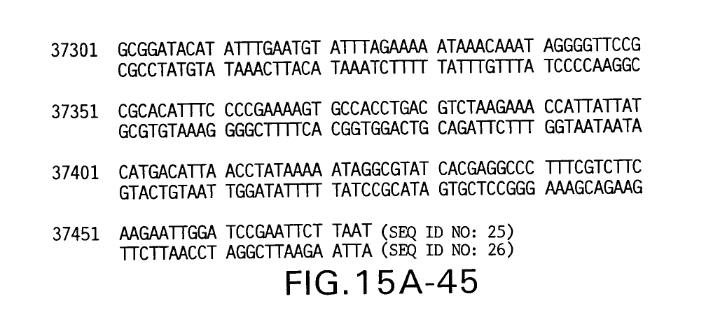

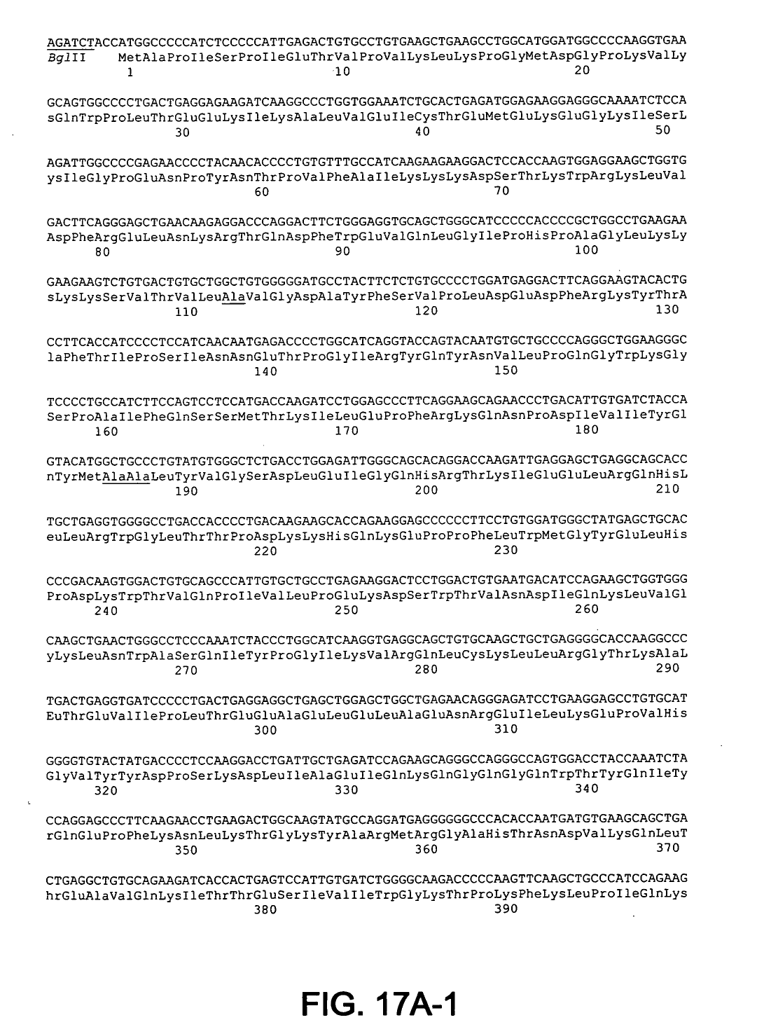

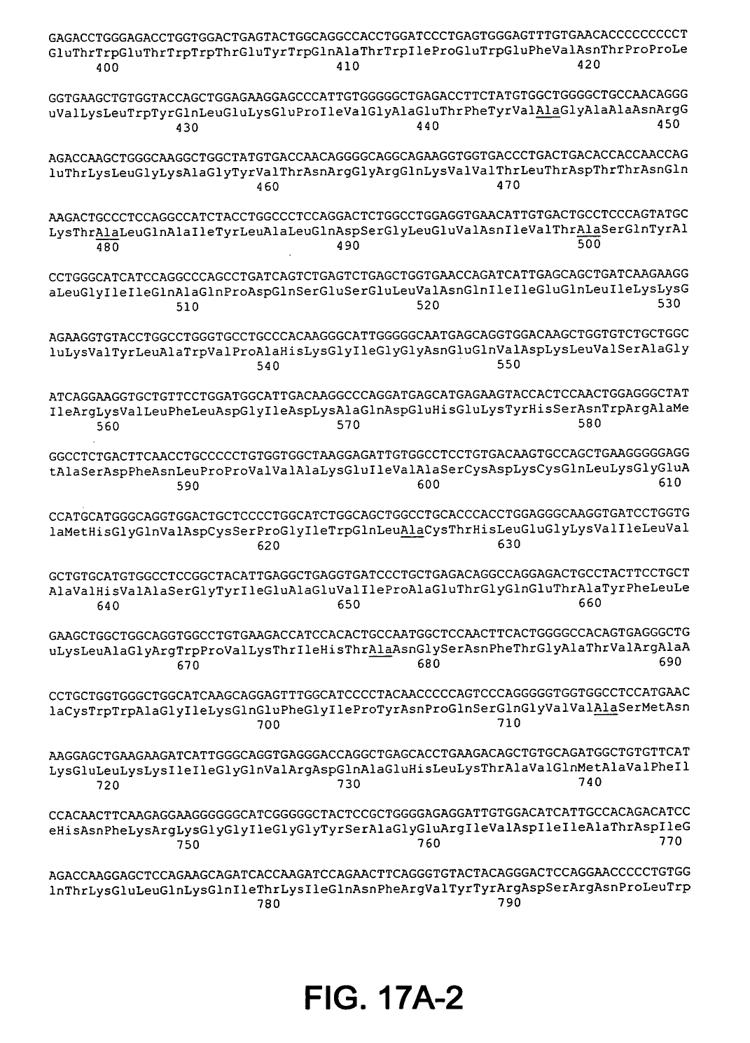

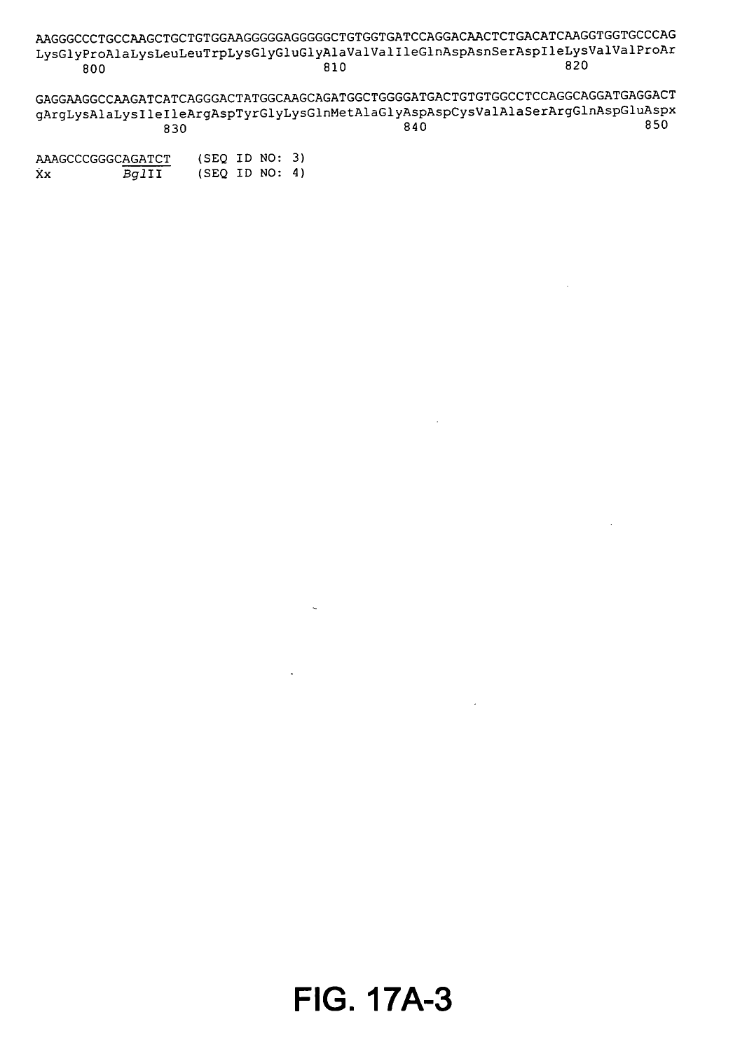

The present invention also relates to a series of MRKAd5pol-based adenoviral vaccines which are shown herein to generate cellular immune responses subsequent to administration in mice and non-human primate studies. Several of the MRKAd5pol series are exemplified herein. One such adenoviral vector is referred to as MRKAd5hCMV-inact opt pol(E3+), which comprises the MRKAd5 backbone, the hCMV promoter (no intron A), an inactivated pol transgene, and contains the Ad5 E3 gene in the adenoviral backbone. A second exemplified pre-adenovirus plasmid and concomitant virus is referred to as MRKAd5hCMV-inact opt pol(E3−), which is identical to the former adenoviral vector except that the E3 is deleted. Both constructions contain a codon optimized, inactivated version of HIV-1 Pol, wherein at least the entire coding region is disclosed herein as SEQ ID NO:3 and the expressed protein is shown as SEQ ID NO:4; see also FIGS. 17A-1 to 17A-3 and Table 1, which show targeted deletion for inactivated pol. This and other preferred codon optimized versions of HIV Pol as disclosed herein are essentially as described in U.S. application Ser. No. 09/745,221, filed Dec. 21, 2000 and PCT International Application PCT/US00/34724, also filed Dec. 21, 2000, both documents which are hereby incorporated by reference. As disclosed in the above-mentioned documents, the open reading frame for these codon-optimized HIV-1 Pol-based DNA vaccines are represented by codon optimized DNA molecules encoding codon optimized HIV-1 Pol (e.g. SEQ ID NO:2), codon optimized HIV-1 Pol fused to an amino terminal localized leader sequence (e.g. SEQ ID NO:6), and especially preferable, and exemplified by the MRKAd5-Pol construct in e.g., Example 19, biologically inactivated pol (“inact opt Pol”; e.g., SEQ ID NO:4) which is devoid of significant PR, RT, RNase or IN activity associated with wild type Pol. In addition, a construct related to SEQ ID NO:4 is contemplated which contains a leader peptide at the amino terminal region of the IA Pol protein. A specific construct is ligated within an appropriate DNA plasmid vector containing regulatory regions operatively linked to the respective HIV-1 Pol coding region, with or without a nucleotide sequence encoding a functional leader peptide. To this end, various HV-1 Pol constructs disclosed herein relate to open reading frames for cloning to the enhanced first generation Ad vectors of the present invention (such a series of MRKAd5pol adenoviral vaccine vectors), including but not limited to wild type Pol (comprising the DNA molecule encoding WT opt Pol, as set forth in SEQ ID NO:2), tPA-opt WTPol, (comprising the DNA molecule encoding tpA Pol, as set forth in SEQ ID NO:6), inact opt Pol (comprising the DNA molecule encoding IA Pol, as set forth in SEQ ID NO:4), and tPA-inact opt Pol, (comprising the DNA molecule encoding tPA-inact opt Pol, as set forth in SEQ ID NO: 8). The pol-based versions of enhanced first generation adenovirus vaccines elicit CTL and Th cellular immune responses upon administration to the host, including primates and especially humans. As noted in the above, an effect of the cellular immune-directed vaccines of the present invention should be a lower transmission rate to previously uninfected individuals and/or reduction in the levels of the viral loads within an infected individual, so as to prolong the asymptomatic phase of HIV-1 infection.

The present invention further relates to a series of MRKAd5nef-based adenoviral vaccines which, similar to HIV gag and pol antigens, generate cellular immune responses subsequent to administration in mice and non-human primate studies. The MRKAd5nef series are exemplified herein by utilizing the improved MRK adenoviral backbone in combination with modified versions of HIV nef. These exemplified MRKAd5nef vectors are as follows: (1) MRKAd5hCMV-nef(G2A,LLAA) (E3+), which comprises the improved MRKAd5 backbone, a human CMV promoter an intact Ad5 E3 gene and a modified nef gene: (2) MRKAd5mCMV-nef(G2A,LLAA) (E3+), which is the same as (1) above but substituting a murine CMV promoter for a human CMV promoter; and (3) MRKAd5mCMV-tpanef(LLAA) (E3+), which is the same as (2) except that the nef transgene is tpanef(LLAA). Codon optimized versions of HIV-1 Nef and HIV-1 Nef modifications are essentially as described in U.S. application Ser. No. 09/738,782, filed Dec. 15, 2000 and PCT International Application PCT/US00/34162, also filed Dec. 15, 2000, both documents which are hereby incorporated by reference. Particular embodiments of codon optimized Nef and Nef modifications relate to a DNA molecule encoding HIV-1 Nef from the HIV-1 jfrl isolate wherein the codons are optimized for expression in a mammalian system such as a human. The DNA molecule which encodes this protein is disclosed herein as SEQ ID NO:9, while the expressed open reading frame is disclosed herein as SEQ ID NO:10. Another embodiment of Nef-based coding regions for use in the adenoviral vectors of the present invention comprise a codon optimized DNA molecule encoding a protein containing the human plasminogen activator (tpa) leader peptide fused with the NH₂-terminus of the HIV-1 Nef polypeptide. The DNA molecule which encodes this protein is disclosed herein as SEQ ID NO:11, while the expressed open reading frame is disclosed herein as SEQ ID NO:12. Another modified Nef optimized coding region relates to a DNA molecule encoding optimized HIV-1 Nef wherein the open reading frame codes for modifications at the amino terminal myristylation site (Gly-2 to Ala-2) and substitution of the Leu-174-Leu-175 dileucine motif to Ala-174-Ala-175, herein described as opt nef (G2A, LLAA). The DNA molecule which encodes this protein is disclosed herein as SEQ ID NO: 13, while the expressed open reading frame is disclosed herein as SEQ ID NO: 14. MRKAd5nef vectors (1) MRKAd5hCMV-nef(G2A,LLAA) (E3+) and (2) MRKAd5mCMV-nef(G2A,LLAA) (E3+) contain this transgene. An additional embodiment relates to a DNA molecule encoding optimized HIV-1 Nef wherein the amino terminal myristylation site and dileucine motif have been deleted, as well as comprising a tPA leader peptide. This DNA molecule, opt tpanef (LLAA), comprises an open reading frame which encodes a Nef protein containing a tPA leader sequence fused to amino acid residue 6-216 of HIV-1 Nef (jfrl), wherein Leu-174 and Leu-175 are substituted with Ala-174 and Ala-175, herein referred to as opt tpanef (LLAA) is disclosed herein as SEQ ID NO:15, while the expressed open reading frame is disclosed herein as SEQ ID NO:16. The MRKAd5nef vector “MRKAd5mCMV-tpanef(LLAA) (E3+)” contains this transgene.

Along with the improved MRKAd5gag adenovirus vaccine vector described herein, generation of a MRKAd5pol and MRKAd5nef adenovirus vector provide for enhanced HIV vaccine capabilities. Namely, the generation of this trio of adenoviral vaccine vectors, all shown to generate effective cellular immune responses subsequent to host administration, provide for the ability to administer these vaccine candidates not only alone, but preferably as part of a divalent (i.e., gag and nef, gag and pol, or pol and nef components) or a trivalent vaccine (i.e., gag, pol and nef components). Therefore, a preferred aspect of the present invention are vaccine formulations and associated methods of administration and concomitant generation of host cellular immune responses associated with formulating three separate series of MRKAd5-based adenoviral vector vaccines. Of course, this MRKAd5 vaccine series based on distinct HIV antigens promotes expanded opportunities for formulation of a divalent or trivalent vaccine, or possibly administration of separate formulations of one or more monovalent or divalent formulations within a reasonable window of time. It is also within the scope of the present invention to embark on combined modality regimes which include multiple but distinct components from a specific antigen. An example, but certainly not a limitation, would be separate MRKAd5pol vectors, with one vaccine vector expressing wild type Pol (SEQ ID NO:2) and another MRKAd5pol vector expressing inactivated Pol (SEQ ID NO:6). Another example might be separate MRKAd5nef vectors, with one vaccine vector expressing the tPA/LLAA version of Nef (SEQ ID NO: 16) and another MRKAd5nef vector expressing the G2A,LLAA modified version of Nef (SEQ ID NO: 14). Therefore, the MRKAd5 adenoviral vectors of the present invention may be used in combination with multiple, distinct HIV antigen classes. Each HIV antigen class is subject to sequence manipulation, thus providing for a multitude of potential vaccine combinations; and such combinations are within the scope of the present invention. The utilization of such combined modalities vaccine formulation and administration increase the probability of eliciting an even more potent cellular immune response when compared to inoculation with a single modality regimen.

The present invention also relates to application of a mono-, dual-, or tri-modality administration regime of the MRKAd5gag, pol and nef adenoviral vaccine series in a prime/boost vaccination schedule. This prime/boost schedule may include any reasonable combination of the MRKAd5gag, pol and nef adenoviral vaccine series disclosed herein. In addition, a prime/boost regime may also involve other viral and/or non-viral DNA vaccines. A preferable addition to an adenoviral vaccine vector regime includes but is not limited to plasmid DNA vaccines, especially DNA plasmid vaccines that contain at least one of the codon optimized gag, pol and nef constructions, as disclosed herein.

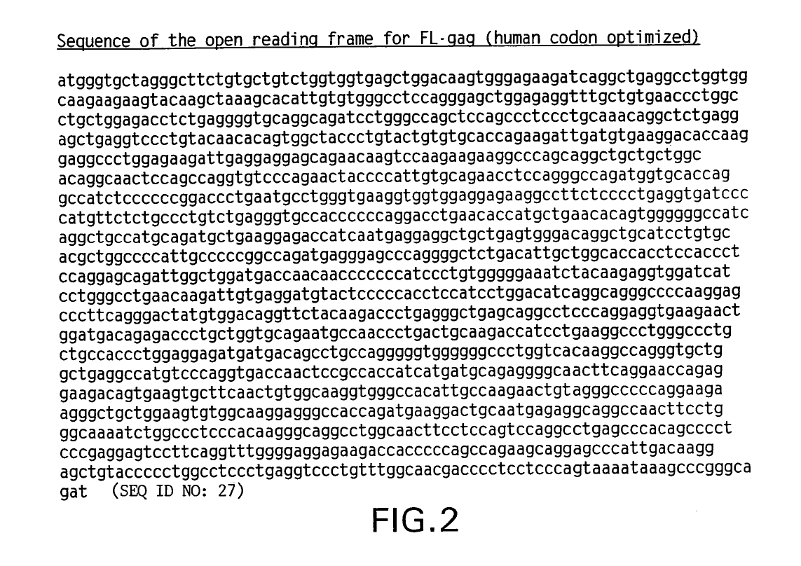

Therefore, one aspect of this invention is the administration of the adenoviral vector containing the optimized gag gene in a prime/boost regiment in conjunction with a plasmid DNA encoding gag. To distinguish this plasmid from the adenoviral-containing shuttle plasmids used in the construction of an adenovirus vector, this plasmid will be referred to as a “vaccine plasmid” or “DNA plasmid vaccine”. Preferred vaccine plasmids for use in this administration protocol are disclosed in pending U.S. patent application Ser. No. 09/017,981, filed Feb. 3, 1998 and WO98/34640, published Aug. 13, 1998, both of which are hereby incorporated by reference. Briefly, the preferred vaccine plasmid is designated V1Jns-FLgag, which expresses the same codon-optimized gag gene as the adenoviral vectors of this invention (see FIG. 2 for the nucleotide sequence of the exemplified optimized codon version of full length p55 gag). The vaccine plasmid backbone, designated V1Jns contains the CMV immediate-early (IE) promoter and intron A, a bovine growth hormone-derived polyadenylation and transcription termination sequence as the gene expression regulatory elements, and a minimal pUC backbone; see Montgomery et al., 1993, DNA Cell Biol. 12:777-783. The pUC sequence permits high levels of plasmid production in E. coli and has a neomycin resistance gene in place of an ampicillin resistance gene to provide selected growth in the presence of kanamycin. Alternatively, a vaccine plasmid which has the CMV promoter deleted of intron A can be used. Those of skill in the art will recognize that alternative vaccine plasmid vectors may be easily substituted for these specific constructs, and this invention specifically envisions use of such alternative plasmid DNA vaccine vectors.

Another aspect of the present invention is a prime/boost regimen which includes a vaccine plasmid which encodes an HIV pol antigen, preferably a codon optimized form of pol and also preferably a vaccine plasmid which comprises a nucleotide sequence which encodes a Pol antigen selected from the group of Pol antigens as shown in SEQ ID NOs: 2, 4, 6 and 8. The variety of potential DNA plasmid vaccines which encode various biologically active forms of HIV-1 Pol, wherein administration, intracellular delivery and expression of the HIV-1 Pol gene of interest elicits a host CTL and Th response. The preferred synthetic DNA molecules of the present invention encode codon optimized wild type Pol (without Pro activity) and various codon optimized inactivated HIV-1 Pol proteins. The HIV-1 pol open reading disclosed herein are especially preferred for pharmaceutical uses, especially for human administration as delivered via a recombinant adenoviral vaccine, especially an enhanced first generation recombinant adenoviral vaccine as described herein. Several embodiments of this portion of the invention are provided in detail below, namely DNA molecules which comprise a HIV-1 pol open reading frame, whether encoding full length pol or a modification or fusion as described herein, wherein the codon usage has been optimized for expression in a mammal, especially a human. Again, these DNA sequences are positioned appropriately within a recombinant adenoviral vector, such as the exemplified recombinant adenoviral vector described herein, so as to promote expression of the respective HIV-1 Pol gene of interest, and subsequent to administration, elicit a host CTL and Th response. Again, these preferred, but in no way limiting, pol genes are as disclosed herein and essentially as described in U.S. application Ser. No. 09/745,221, filed Dec. 21, 2000 and PCT International Application PCT/US00/34724, also filed Dec. 21, 2000, both documents which are hereby incorporated by reference.

A third series of vaccine plasmids which are useful in a combined modality and/or prime/boost regimen are vaccine plasmids which encode an HIV nef antigen or biologically and/or immunologically relevant modification thereof. As noted elsewhere, preferred vaccine plasmids contain a codon optimized form of nef and also preferably comprise a nucleotide sequence which encodes a Nef antigen selected from the group of Nef antigens as shown in SEQ ID NOs: 10, 12, 14 and 16. These preferred nef coding regions are disclosed herein, as well as being described in U.S. application Ser. No. 09/738,782, filed Dec. 15, 2000 and PCT International Application PCT/US00/34162, also filed Dec. 15, 2000, both documents which are hereby incorporated by reference.

Therefore, the adenoviral vaccines and plasmid DNA vaccines of this invention may be administered alone, or may be part of a prime and boost administration regimen. A mixed modality priming and booster inoculation scheme will result in an enhanced immune response, particularly is pre-existing anti-vector immune responses are present. This one aspect of this invention is a method of priming a subject with the plasmid vaccine by administering the plasmid vaccine at least one time, allowing a predetermined length of time to pass, and then boosting by administering the adenoviral vaccine. Multiple primings typically, 1-4, are usually employed, although more may be used. The length of time between priming and boost may typically vary from about four months to a year, but other time frames may be used. In experiments with rhesus monkeys, the animals were primed four times with plasmid vaccines, then were boosted 4 months later with the adenoviral vaccine. Their cellular immune response was notably higher than that of animals which had only received adenoviral vaccine. The use of a priming regimen may be particularly preferred in situations where a person has a preexisting anti-adenovirus immune response.

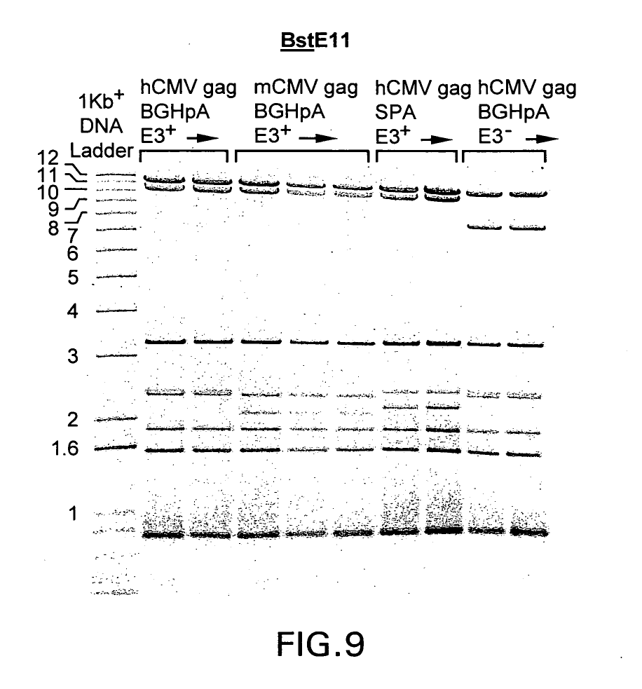

Furthermore and in the alternative, multiple HIV-1 viral antigens, such as the MRKAd5 adenoviral vaccines disclosed herein, may be ligated into a proper shuttle plasmid for generation of a pre-adenoviral plasmid comprising multiple open reading frames. For example a trivalent vector may comprise a gag-pol-nef fusion, in either a E3(−) or E3(+) background, preferably a E3 deleted backbone, or possible a “2+1” divalent vaccine, such as a gag-pol fusion (i.e., codon optimized p55 gag and inactivated optimized pol; Example 29 and Table 25) within the same MRKAd5 backbone, with each open reading frame being operatively linked to a distinct promoter and transcription termination sequence. Alternatively, the two open reading frames may be operatively linked to a single promoter, with the open reading frames operatively linked by an internal ribosome entry sequence (IRES), as disclosed in International Publication No. WO 95/24485, which is hereby incorporated by reference. FIG. 9 shows that the use of multiple promoters and termination sequences provide for similar growth properties, while FIG. 28 shows that these MRKAd5gag-based vectors are also stable at least through passage 21. In the absence of the use of IRES-based technology, it is preferred that a distinct promoter be used to support each respective open reading frame, so as to best preserve vector stability. As examples, and certainly not as limitations, potential multiple transgene vaccines may include a three transgene vector such as hCMV-gagpol-bGHpA+mCMV-nef-SPA in an E3 deleted backbone or hCMV-gagpol-bGHpA+mCMV-nef-SPA(E3+). Potential “2+1” divalent vaccines of the present invention might be a hCMV-gag-bGHpA+mCMV-nef-SPA in an E3+backbone (vector #1) in combination with hCMV-pol-bGHpA in an E3+ backbone (vector #2), with all transgenes in the E1 parallel orientation. Fusion constructs other than the gag-pol fusion described above are also suitable for use in various divalent vaccine strategies and can be composed of any two HIV antigens fused to one another (e.g., nef-pol and gag-nef). These adenoviral compositions are, as above, preferably delivered along with an adenoviral composition comprising an additional HIV antigen in order to diversify the immune response generated upon administration. Therefore, a multivalent vaccine delivered in a single, or possible second, adenoviral vector is certainly contemplated as part of the present invention. Again, this mode of administration is another example of whereby an efficaceous adenovirus-based HIV-1 vaccine may be administered via a combined modality regime. It is important to note, however, that in terms of deciding on an insert for the disclosed adenoviral vectors, due consideration must be dedicated to the effective packaging limitations of the adenovirus vehicle. Adenovirus has been shown to exhibit an upper cloning capacity limit of approximately 105% of the wildtype Ad5 sequence.

Regardless of the gene chosen for expression, it is preferred that the sequence be “optimized” for expression in a human cellular environment. A “triplet” codon of four possible nucleotide bases can exist in 64 variant forms. That these forms provide the message for only 20 different amino acids (as well as transcription initiation and termination) means that some amino acids can be coded for by more than one codon. Indeed, some amino acids have as many as six “redundant”, alternative codons while some others have a single, required codon. For reasons not completely understood, alternative codons are not at all uniformly present in the endogenous DNA of differing types of cells and there appears to exist variable natural hierarchy or “preference” for certain codons in certain types of cells. As one example, the amino acid leucine is specified by any of six DNA codons including CTA, CTC, CTG, CTT, TTA, and TTG (which correspond, respectively, to the mRNA codons, CUA, CUC, CUG, CUU, UUA and UUG). Exhaustive analysis of genome codon frequencies for microorganisms has revealed endogenous DNA of E. coli most commonly contains the CTG leucine-specifying codon, while the DNA of yeasts and slime molds most commonly includes a TTA leucine-specifying codon. In view of this hierarchy, it is generally held that the likelihood of obtaining high levels of expression of a leucine-rich polypeptide by an E. coli host will depend to some extent on the frequency of codon use. For example, a gene rich in TTA codons will in all probability be poorly expressed in E. coli, whereas a CTG rich gene will probably highly express the polypeptide. Similarly, when yeast cells are the projected transformation host cells for expression of a leucine-rich polypeptide, a preferred codon for use in an inserted DNA would be TTA.

The implications of codon preference phenomena on recombinant DNA techniques are manifest, and the phenomenon may serve to explain many prior failures to achieve high expression levels of exogenous genes in successfully transformed host organisms—a less “preferred” codon may be repeatedly present in the inserted gene and the host cell machinery for expression may not operate as efficiently. This phenomenon suggests that synthetic genes which have been designed to include a projected host cell's preferred codons provide a preferred form of foreign genetic material for practice of recombinant DNA techniques. Thus, one aspect of this invention is an adenovirus vector or adenovirus vector in some combination with a vaccine plasmid where both specifically include a gene which is codon optimized for expression in a human cellular environment. As noted herein, a preferred gene for use in the instant invention is a codon-optimized HIV gene and, particularly, HIV gag, pol or nef.

Adenoviral vectors in accordance with the instant invention can be constructed using known techniques, such as those reviewed in Hitt et al, 1997 “Human Adenovirus Vectors for Gene Transfer into Mammalian Cells” Advances in Pharmacology 40:137-206, which is hereby incorporated by reference.

In constructing the adenoviral vectors of this invention, it is often convenient to insert them into a plasmid or shuttle vector. These techniques are known and described in Hitt et al., supra. This invention specifically includes both the adenovirus and the adenovirus when inserted into a shuttle plasmid.

Preferred shuttle vectors contain an adenoviral portion and a plasmid portion. The adenoviral portion is essentially the same as the adenovirus vector discussed supra, containing adenoviral sequences (with non-functional or deleted E1 and E3 regions) and the gene expression cassette, flanked by convenient restriction sites. The plasmid portion of the shuttle vector often contains an antibiotic resistance marker under transcriptional control of a prokaryotic promoter so that expression of the antibiotic does not occur in eukaryotic cells. Ampicillin resistance genes, neomycin resistance genes and other pharmaceutically acceptable antibiotic resistance markers may be used. To aid in the high level production of the polynucleotide by fermentation in prokaryotic organisms, it is advantageous for the shuttle vector to contain a prokaryotic origin of replication and be of high copy number. A number of commercially available prokaryotic cloning vectors provide these benefits. It is desirable to remove non-essential DNA sequences. It is also desirable that the vectors not be able to replicate in eukaryotic cells. This minimizes the risk of integration of polynucleotide vaccine sequences into the recipients' genome. Tissue-specific promoters or enhancers may be used whenever it is desirable to limit expression of the polynucleotide to a particular tissue type.

In one embodiment of this invention, the pre-plasmids (e.g., pMRKAd5pol, pMRKAd5nef and pMRKAd5gag were generated by homologous recombination using the MRKHVE3 (and MRKHVO for the E3− version) backbones and the appropriate shuttle vector, as shown for pMRKAd5pol in FIG. 22 and for pMRKAd5nef in FIG. 23. The plasmid in linear form is capable of replication after entering the PER.C6® cells and virus is produced. The infected cells and media were harvested after viral replication was complete.

Viral vectors can be propagated in various E1 complementing cell lines, including the known cell lines 293 and PER.C6®. Both these cell lines express the adenoviral E1 gene product. PER.C6® is described in WO 97/00326 (published Jan. 3, 1997) and issued U.S. Pat. No. 6,033,908, both of which are hereby incorporated by reference. It is a primary human retinoblast cell line transduced with an E1 gene segment that complements the production of replication deficient (FG) adenovirus, but is designed to prevent generation of replication competent adenovirus by homologous recombination. Cells of particular interest have been stably transformed with a transgene that encodes the AD5E1A and E1B gene, like PER.C6®, from 459 bp to 3510 bp inclusive. 293 cells are described in Graham et al., 1977 J. Gen. Virol 36:59-72, which is hereby incorporated by reference. As stated above, consideration must be given to the adenoviral sequences present in the complementing cell line used. It is important that the sequences not overlap with that present in the vector if the possibility of recombination is to be minimized.

It has been found that vectors generated in accordance with the above description are more effective in inducing an immune response and, thus, constitute very promising vaccine candidates. More particularly, it has been found that first generation adenoviral vectors in accordance with the above description carrying a codon-optimized HIV gag gene, regulated with a strong heterologous promoter can be used as human anti-HIV vaccines, and are capable of inducing immune responses.

Standard techniques of molecular biology for preparing and purifying DNA constructs enable the preparation of the DNA immunogens of this invention.

A vaccine composition comprising an adenoviral vector in accordance with the instant invention may contain physiologically acceptable components, such as buffer, normal saline or phosphate buffered saline, sucrose, other salts and polysorbate. One preferred formulation has: 2.5-10 mM TRIS buffer, preferably about 5 mM TRIS buffer; 25-100 mM NaCl, preferably about 75 mM NaCl; 2.5-10% sucrose, preferably about 5% sucrose; 0.01-2 mM MgCl₂; and 0.001%-0.01% polysorbate 80 (plant derived). The pH should range from about 7.0-9.0, preferably about 8.0. One skilled in the art will appreciate that other conventional vaccine excipients may also be used it make the formulation. The preferred formulation contains 5 mM TRIS, 75 mM NaCl, 5% sucrose, ImM MgCl₂, 0.005% polysorbate 80 at pH 8.0 This has a pH and divalent cation composition which is near the optimum for Ad5 stability and minimizes the potential for adsorption of virus to a glass surface. It does not cause tissue irritation upon intramuscular injection. It is preferably frozen until use.

The amount of adenoviral particles in the vaccine composition to be introduced into a vaccine recipient will depend on the strength of the transcriptional and translational promoters used and on the immunogenicity of the expressed gene product. In general, an immunologically or prophylactically effective dose of 1×10⁷ to 1×10¹² particles and preferably about 1×10¹⁰ to 1×10¹¹ particles is administered directly into muscle tissue. Subcutaneous injection, intradermal introduction, impression through the skin, and other modes of administration such as intraperitoneal, intravenous, or inhalation delivery are also contemplated. It is also contemplated that booster vaccinations are to be provided. Following vaccination with HIV adenoviral vector, boosting with a subsequent HIV adenoviral vector and/or plasmid may be desirable. Parenteral administration, such as intravenous, intramuscular, subcutaneous or other means of administration of interleukin- 12 protein, concurrently with or subsequent to parenteral introduction of the vaccine compositions of this invention is also advantageous.

The adenoviral vector and/or vaccine plasmids of this invention polynucleotide may be unassociated with any proteins, adjuvants or other agents which impact on the recipients' immune system. In this case, it is desirable for the vector to be in a physiologically acceptable solution, such as, but not limited to, sterile saline or sterile buffered saline. Alternatively, the vector may be associated with an adjuvant known in the art to boost immune responses (i.e., a “biologically effective” adjuvant), such as a protein or other carrier. Vaccine plasmids of this invention may, for instance, be delivered in saline (e.g., PBS) with or without an adjuvant. Preferred adjuvants are Alum or CRL1005 Block Copolymer. Agents which assist in the cellular uptake of DNA, such as, but not limited to, calcium ions, may also be used to advantage. These agents are generally referred to herein as transfection facilitating reagents and pharmaceutically acceptable carriers. Techniques for coating microprojectiles coated with polynucleotide are known in the art and are also useful in connection with this invention.

This invention also includes a prime and boost regimen wherein a first adenoviral vector is administered, then a booster dose is given. The booster dose may be repeated at selected time intervals. Alternatively, a preferred inoculation scheme comprises priming with a first adenovirus serotype and then boosting with a second adenovirus serotype. More preferably, the inoculation scheme comprises priming with a first adenovirus serotype and then boosting with a second adenovirus serotype, wherein the first and second adenovirus serotypes are classified within separate subgroups of adenoviruses. The above prime/boost schemes are particularly preferred in those situations where a preexisting immunity is identified to the adenoviral vector of choice. In this type of scheme, the individual or population of individuals is primed with an adenovirus of a serotype other than that to which the preexisting immunity is identified. This enables the first adenovirus to effectuate sufficient expression of the transgene while evading existing immunity to the second adenovirus (the boosting adenovirus) and, further, allows for the subsequent delivery of the transgene via the boosting adenovirus to be more effective. Adenovirus serotype 5 is one example of a virus to which such a scheme might be desirable. In accordance with this invention, therefore, one might decide to prime with a non-group C adenovirus (e.g., Ad12, a group A adenovirus, Ad24, a group D adenovirus, or Ad35, a group B adenovirus) to evade anti-Ad5 immunity and then boost with Ad5, a group C adenovirus. Another preferred embodiment involves administration of a different adenovirus (including non-human adenovirus) vaccine followed by administration of the adenoviral vaccines disclosed. In the alternative, a viral antigen of interest can be first delivered via a viral vaccine other than an adenovirus-based vaccine, and then followed with the adenoviral vaccine disclosed. Alternative viral vaccines include but are not limited to pox virus and venezuelan equine encephilitis virus.

A large body of human and animal data supports the importance of cellular immune responses, especially CTL in controlling (or eliminating) HIV infection. In humans, very high levels of CTL develop following primary infection and correlate with the control of viremia. Several small groups of individuals have been described who are repeatedly exposed to HIV by remain uninfected; CTL has been noted in several of these cohorts. In the SIV model of HIV infection, CTL similarly develops following primary infection, and it has been demonstrated that addition of anti-CD8 monoclonal antibody abrogated this control of infection and leads to disease progression. This invention uses adenoviral vaccines alone or in combination with plasmid vaccines to induce CTL.

The following non-limiting Examples are presented to better illustrate the invention.

### EXAMPLE 1

### Removal of the Intron A Portion of the hCMV Promoter

GMP grade pVIJnsHIVgag was used as the starting material to amplify the hCMV promoter. PVIJnsHIVgag is a plasmid comprising the CMV immediate-early (IE) promoter and intron A, a full-length codon-optimized HIV gag gene, a bovine growth hormone-derived polyadenylation and transcriptional termination sequence, and a minimal pUC backbone; see Montgomery et al., supra for a description of the plasmid backbone. The amplification was performed with primers suitably positioned to flank the hCMV promoter. A 5′ primer was placed upstream of the Msc1 site of the hCMV promoter and a 3′ primer (designed to contain the Bg/II recognition sequence) was placed 3′ of the hCMV promoter. The resulting PCR product (using high fidelity Taq polymerase) which encompassed the entire hCMV promoter (minus intron A) was cloned into TOPO PCR blunt vector and then removed by double digestion with Msc1 and Bg/II. This fragment was then cloned back into the original GMP grade pV1JnsHfVgag plasmid from which the original promoter, intron A, and the gag gene were removed following Msc1 and Bg/II digestion. This ligation reaction resulted in the construction of a hCMV promoter (minus intron A)+bGHpA expression cassette within the original pV1JnsHlVgag vector backbone. This vector is designated pV1JnsCMV(no intron).

The FLgag gene was excised from pV1JnsHfVgag using Bg/II digestion and the 1,526 bp gene was gel purified and cloned into pV1JnsCMV(no intron) at the Bg/II site. Colonies were screened using Sma1 restriction enzymes to identify clones that carried the Flgag gene in the correct orientation. This plasmid, designated pV1JnsCMV(no intron)-FLgag-bGHpA, was fully sequenced to confirm sequence integrity.

Two additional transgenes were also constructed. The plasmid, pV1JnsCMV(no intron)-FLgag-SPA, is identical to pV1JnsCMV(no intron)-FLgag-bGHpA except that the bovine growth hormone polyadenylation signal has been replaced with a short synthetic polyA signal (SPA) of 50 nucleotides in length. The sequence of the SPA is as shown, with the essential components (poly(A) site, (GT)ₙ, and (T)ₙ; respectively) underlined: AATAAAAGATCTITATTTTCATTAGATCTGTGTG TTGGTTTTTGTGTG (SEQ ID NO: 18).

The plasmid, pV1Jns-mCMV-FLgag-bGHpA, is identical to the pV1JnsCMV(no intron)-FLgag-bGHpA except that the hCMV promoter has been removed and replaced with the murine CMV (mCMV) promoter.

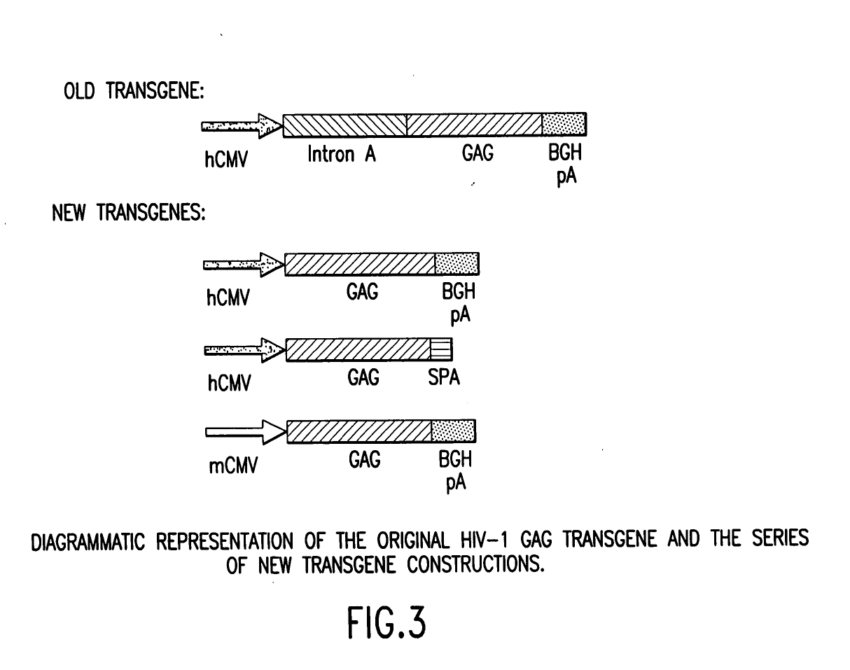

FIG. 3 diagrammatically shows the new transgene constructs in comparison with the original transgene.

### EXAMPLE 2

### Gag Expression Assay for Modified Gag Transgenes

Gag Elisa was performed on culture supernatants obtained from transient tissue culture transfection experiments in which the two new hCMV-containing plasmid constructs, pV1JnsCMV(no intron)-FLgag-bGHpA and pV1JnsCMV(no intron)-FLgag-SPA, both devoid of intron A, were compared to pV1JnsHIVgag which, as noted above possesses the intron A as part of the hCMV promoter. Table 2 below shows the in vitro gag expression data of the new gag plasmids compared with the GMP grade original plasmid. The results displayed in Table 2 show that both of the new hCMV gag plasmid constructs have expression capacities comparable to the original plasmid construct which contains the intron A portion of the hCMV promoter.

TABLE 2
In vitro DNA transfection of original and
new plasmid HIV-1 gag constructs.

| Plasmid | μg gag/10e6 COS cells/5 μg DNA/48 hr |
|---|---|
| HIVFL-gagPR9901^{a} | 10.8 |
| PVIJns-hCMV-FLgag-bGHpA^{b} | 16.6 |
| pV1Jns-hCMV-FLgag-SPA^{b,c} | 12.0 |

^{a}GMP grade pV1Jns-hCMVintronA-FLgag-bGHpA.
^{b}New plasmid constructions that have the intron A portion removed from the hCMV promoter.
^{c}In this construct the bGH terminator has been replaced with the short synthetic polyadenylation signal (SPA)

### EXAMPLE 3

### Rodent (Balb/c) Study for Modified Gag Transgenes

A rodent study was performed on the two new plasmid constructs described above—pV1JnsCMV(no intron)-FLgag-bGHpA and pV1JnsCMV(no intron)-FLgag-SPA—in order to compare them with the construct described above possessing the intron A portion of the CMV promoter, pV1JnsHIVgag. Gag antibody and Elispot responses (described in PCT International Application No. PCT/US00/18332 (WO 01/02607) filed Jul. 3, 2000, claiming priority to U.S. Provisional Application Ser. No. 60/142,631, filed Jul. 6, 1999 and U.S. Application Ser. No. 60/148,981, filed Aug. 13, 1999, all three applications which are hereby incorporated by reference) were measured. The results displayed in Table 3 below, show that the new plasmid constructs behaved equivalently to the original construct in Balb/c mice with respect to their antibody and T-cell responses at both dosages of plasmid DNA tested, 20 μg and 200 μg.

### EXAMPLE 4

TABLE 3
HIV191: Immunogenicity of V1Jns-gag under different
promoter and termination control elements.

| | | Anti-p24 Titers | | | SFC/10\hat{ }6 Cells | | |
|---|---|---|---|---|---|---|---|
| DNA^{a} | Dose, | (3 Wk PD1)^{c} | | | (4 Wk PD1)^{d} | | |
| Promoter/terminator | ug^{b} | GMT | +SE | −SE | Media | gag197-205 | p24 |
| HIVFL-gagPR9901 | 200 | 12800 | 4652 | 3412 | 2(2) | 129(19) | 30(11) |
| (GMP grade) | 20 | 5572 | 1574 | 1227 | 0 | 56(9) | 25(6) |
| pV1Jns-hCMV- | 200 | 11143 | 2831 | 2257 | 0 | 98(5) | 12(6) |
| FL-gag-bGHpA | 20 | 7352 | 2808 | 2032 | 0 | 73(9) | 11(6) |
| pV1Jns-hCMV- | 200 | 16890 | 5815 | 4326 | 1(1) | 94(4) | 26(7) |
| FL-gag-SPA | 20 | 5971 | 5361 | 2825 | 0 | 85(17) | 38(10) |
| Naïve | 0 | 123 | 50 | 36 | 0 | 0 | 0 |

^{a}in PBS
^{b}i.m. Injections into both quads, 50 μL per quad
^{c}n = 10; GMT, geometric mean titer; SE, standard. error
^{d}n = 5, pooled spleens; mean of triplicate wells and standard. deviation. in parentheses;

### Construction of the Modified Shuttle Vector—“MRKpdelE1 Shuttle”

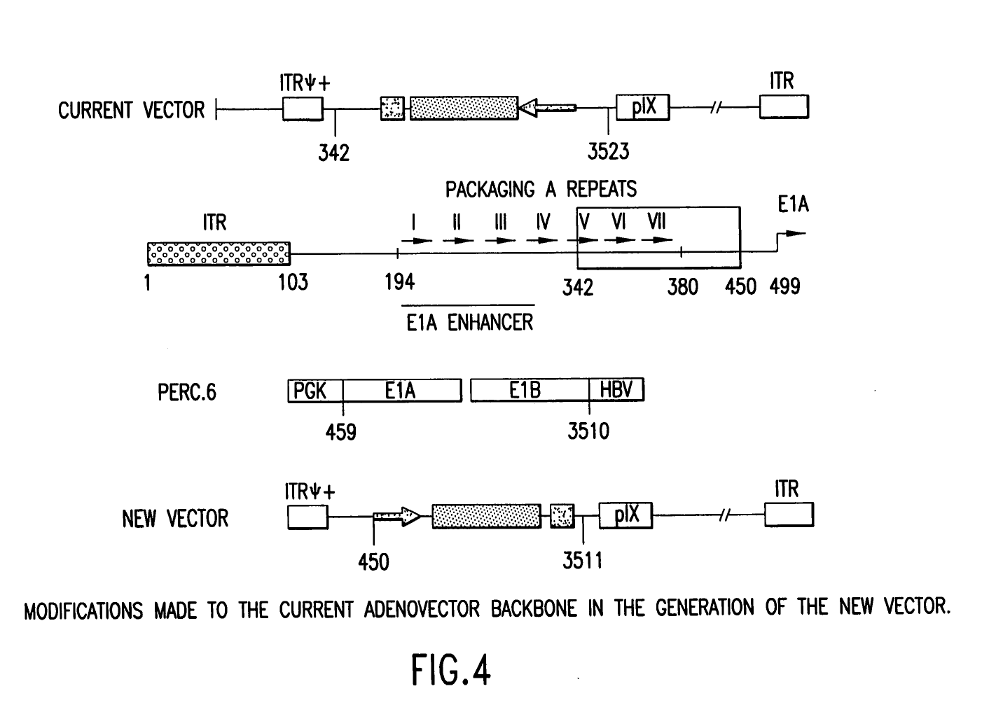

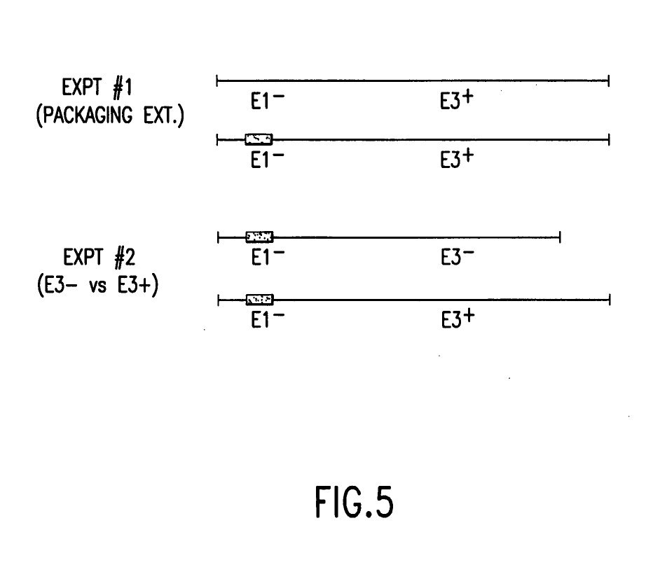

The modifications to the original Ad5 shuttle vector (pdelE1sp1A; a vector comprising Ad5 sequences from basepairs 1-341 and 3524-5798, with a multiple cloning region between nucleotides 341 and 3524 of Ad5, included the following three manipulations carried out in sequential cloning steps as follows:
(1) The left ITR region was extended to include the Pac1 site at the junction between the vector backbone and the adenovirus left ITR sequences. This allow for easier manipulations using the bacterial homologous recombination system.
(2) The packaging region was extended to include sequences of the wild-type (WT) adenovirus from 342 bp to 450 bp inclusive.
(3) The area downstream of pIX was extended 13 nucleotides (i.e., nucleotides 3511-3523 inclusive). These modifications (FIG. 4) effectively reduced the size of the E1 deletion without overlapping with any part of the E1A/E1B gene present in the transformed PER.C6® cell line. All manipulations were performed by modifying the Ad shuttle vector pdelE1sp1A.

Once the modifications were made to the shuttle vector, the changes were incorporated into the original Ad5 adenovector backbones (pAdHVO and pAdHVE3) by bacterial homologous recombination using E. coli BJ5183 chemically competent cells.

### EXAMPLE 5

### Construction of Modified Adenovector Backbones (E3+ and E3−)

The original adenovectors pAdHVO (comprising all Ad5 sequences except those nucleotides encompassing the E1 and E3 regions ) and pADHVE3 (comprising all Ad5 sequences except those nucleotides encompassing the E1 region), were each reconstructed so that they contained the modifications to the E1 region. This was accomplished by digesting the newly modified shuttle vector (MRKpdelE1 shuttle) with Pac1 and BstZ1101 and isolating the 2,734 bp fragment which corresponds to the adenovirus sequence. This fragment was co-transformed with DNA from either Cla1 linearized pAdHVO (E3− adenovector) or Cla1 linearized pAdHVE3 (E3+adenovector) into E. coli BJ5 183 competent cells. At least two colonies from each transformation were selected and grown in Terrific™ broth for 6-8 hours until turbidity was reached. DNA was extracted from each cell pellet and then transformed into E. coli XL1 competent cells. One colony from each transformation was selected and grown for plasmid DNA purification. The plasmid was analyzed by restriction digestions to identify correct clones. The modified adenovectors were designated MRKpAdHVO (E3− plasmid) and MRKpAdHVE3 (E3+ plasmid). Virus from these new adenovectors (MRKHVO and MRKHVE3, respectively) as well as the old version of the adenovectors were generated in the PER.C6° cell lines to accommodate the following series of viral competition experiments. In addition, the multiple cloning site of the original shuttle vector contained ClaI, BamHI, Xho I, EcoRV, HindIII, Sal I, and Bgl II sites. This MCS was replaced with a new MCS containing Not I, Cla I, EcoRV and Asc I sites. This new MCS has been transferred to the MRKpAdHVO and MRKpAdHVE3 pre-plasmids along with the modification made to the packaging region and pIX gene.

### EXAMPLE 6

### Analysis of the Effect of the Packaging Signal Extension

To study the effects of the modifications made to the El deletion region, the viruses obtained from the original backbone (pAdHVE3) and the new backbone (MRKpAdHVE3) were mixed together in equal MOI ratios (1:1 and 5:5) and passaged through several rounds; see FIG. 5, Expt.#1. Both of the viruses in the experiment contained the E3 gene intact and did not contain a transgene. The only difference between the two viruses was within the region of the E1 deletion. Following the coinfection of the viruses at P1 (passage 1), the mixtures were propagated through an additional 4 passages at which time the cells were harvested and the virus extracted and purified by CsCl banding. The viral DNA was extracted and digested with HindIII and the digestion products were then radioactively labeled. For the controls, the respective pre-plasmids (pAdHVE3 (“OLD E3+”); MRKpAdHVE3 (“NEW E3+”)) were also digested with HindIII (and Pac1 to remove the vector backbone) and subsequently labeled with [³³P]dATP. The radioactively labeled digestion products were subjected to gel electrophoresis and the gel was dried down onto Whatman paper before being exposed to autoradiographic film. FIG. 6 clearly shows that the new adenovirus which has the addition made to the packaging signal region has a growth advantage compared with the original adenovirus. In the experiments performed (at either ratio tested), only the digestion bands pertaining to the newly modified virus were present. The diagnostic band of size 3,206 (from the new virus) was clearly present. However, there was no evidence of the diagnostic band of size 2,737 bp expected from the original virus.

### EXAMPLE 7

### Analysis of the Effect of the E3 Gene

The second set of the virus competition study involved mixing equal MOI ratio (1:1) of the newly modified viruses, that obtained from MRKpAdHVO and MRKpAdHVE3 (FIG. 5, Expt. #2). In this set, both viruses had the new modifications made to the E1 deletion. The first virus (that from MRKpAdHVO) does not contain an E3 gene. The second virus (that from MRKpAdHVE3) does contain the E3 gene. Neither of the viruses contain a transgene. Following co-infection of the viruses, the mixtures were propagated through an additional 4 passages at which time the cells were harvested and the total virus extracted and purified by CsCl banding. The viral DNA was extracted and digested with Hind/III and the digestion products were then radioactively labeled. For the controls, the respective pre-plasmids MRKpAdHVO (“NEW E3−”); MRKpAdHVE3 (“NEW E3+”) were also digested with HindIII (and Pac1 to remove the vector backbone) and then labeled with [³³P]dATP. The radioactively labeled digestion products were subjected to gel electrophoresis and the gel was dried down onto Whatman paper before being exposed to autoradiographic film. FIG. 6 shows the results of the viral DNA analysis of the E3+ virus and E3− virus mixing experiment. The diagnostic band corresponding to the E3+ virus (5,665 bp) was present in greater amount compared with the diagnostic band of 3,010 bp corresponding to the E3− virus. This indicates that the virus that contains the E3 gene is able to amplify more rapidly compared with the virus that does not contain an E3 gene. This increased amplification capacity has been confirmed by growth studies; see Table 4 below.

### EXAMPLE 8

### Construction of the New Shuttle Vector Containing Modified Gag Transgene—“MRKpdelE1-CMV(no intron)-FLgag-bGHpA”

The modified plasmid pV1JnsCMV(no intron)-FLgag-bGHpA was digested with Msc1 overnight and then digested with Sfi1 for 2 hours at 50° C. The DNA was then treated with Mungbean nuclease for 30 mins at 30° C. The DNA mixture was desalted using the Qiaex II kit and then Klenow treated for 30 mins at 37° C. to fully blunt the ends of the transgene fragment. The 2,559 bp transgene fragment was then gel purified. The modified shuttle vector (MRKpdelE1 shuttle) was linearized by digestion with EcoRV, treated with calf intestinal phosphatase and the resulting 6,479 bp fragment was then gel purified. The two purified fragments were then ligated together and several dozen clones were screened to check for insertion of the transgene within the shuttle vector. Diagnostic restriction digestion was performed to identify those clones carrying the transgene in the E1 parallel and E1 anti-parallel orientation. This strategy was followed to clone in the other gag transgenes in the MRKpdelE1 shuttle vector.

### EXAMPLE 9

### Construction of the MRK FG Adenovectors

The shuttle vector containing the HIV-1 gag transgene in the E1 parallel orientation, MRKpdelE1-CMV(no intron)-FLgag-bGHpA, was digested with Pac1. The reaction mixture was digested with BsJZ171. The 5,291 bp fragment was purified by gel extraction. The MRKpAdHVE3 plasmid was digested with Cla1 overnight at 37° C. and gel purified. About 100 ng of the 5,290 bp shuttle+transgene fragment and ˜100 ng of linearized MRKpAdHVE3 DNA were co-transformed into E. coli BJ5183 chemically competent cells. Several clones were selected and grown in 2 ml Terrific™ broth for 6-8 hours, until turbidity was reached. The total DNA from the cell pellet was purified using Qiagen alkaline lysis and phenol chloroform method. The DNA was precipitated with isopropanol and resuspended in 20 μl dH₂0. A 2 μl aliquot of this DNA was transformed into E. coli XL-1 competent cells. A single colony from each separate transformation was selected and grown overnight in 3 ml LB+100 gg/ml ampicillin. The DNA was isolated using Qiagen columns. A positive clone was identified by digestion with the restriction enzyme BstEII which cleaves within the gag gene as well as the plasmid backbone. The pre-plasmid clone is designated MRKpAdHVE3+CMV(no intron)-FLgag-bGHpA and is 37,498 bp in size. This strategy was followed to generate E3− and E3+ versions of each of the other gag transgene constructions in both E1 parallel and E1 anti-parallel versions. FIGS. 7A, 7B and 7C show the various combinations of adenovectors constructed.

### EXAMPLE 10

### Plasmid Competition Studies

A series of plasmid competition studies was carried out. Briefly, the screening of the various combinations of new constructs was performed by mixing equal amounts of each of two competing plasmids. In the experiment shown in FIG. 8A, plasmids containing the same transgene but in different orientations were mixed together to create a “competition” between the two plasmids. The aim was to look at the effects of transgene orientation. In the experiment shown in FIG. 8B, plasmids containing different polyadenylation signals (but in the same orientation) were mixed together in equal amounts. The aim was to assess effects of polyA signals. Following the initial transfection, the virus was passaged through ten rounds and the viral DNA analyzed by radioactive restriction analysis.

Analysis of the viral species from the plasmid mixing experiment (FIG. 8A) showed that adenovectors which had the transgene inserted in the E1 parallel orientation amplified better and were able to out-compete the adenovirus which had the transgene inserted in the E1 anti-parallel orientation. Viral DNA analysis of the mixtures at passage 3 and certainly at passage 6, showed a greater ratio of the virus carrying the transgene in the E1 parallel orientation compared with the E1 antiparallel version. By passage 10, the only viral species observed was the adenovector with the transgene in the E1 parallel orientation for both transgenes tested (hCMV(no intron)-FLgag-bGHpA and hCMV(no intron)-FLgag-SPA).

Analysis of the viral species from the plasmid mixing experiment #2 (FIG. 8B) at passages 3 and 6 showed that the polyadenylation signals tested (bGHpA and SPA) did not have an effect on the growth of the virus. Even at passage 10 the two viral species in the mixture were still present in equal amounts.

### EXAMPLE 11

### Virus Generation of an Enhanced Adenoviral Construct—“MRK Ad5 HIV-1gag”

The results obtained from the competition study allowed us to make the following conclusions: (1) The packaging signal extension is beneficial; (2) Presence of E3 does enhance viral growth; (3) E1 parallel orientation is recommended; and (4) PolyA signals have no effect on the growth of the adenovirus.

MRK Ad5 HIV-1 gag exhibited the most desirable results. This construct contains the hCMV(no intron)-FLgag-bGHpA transgene inserted into the new E3+ adenovector backbone, MRKpAdHVE3, in the E1 parallel orientation. We have designated this adenovector MRK Ad5 HIV-1 gag. This construct was prepared as outlined below:

The pre-plasmid MRKpAdHVE3+CMV(no intron)-FLgag-bGHpA was digested was Pac1 to release the vector backbone and 3.3 μg was transfected by calcium phosphate method (Amersham Pharmacia Biotech.) in a 6 cm dish containing PER.C6® cells at ˜60% confluence. Once CPE was reached (7-10 days), the culture was freeze/thawed three times and the cell debris pelleted. 1 ml of this cell lysate was used to infect into a 6 cm dish containing PER.C6® cells at 80-90% confluence. Once CPE was reached, the culture was freeze/thawed three times and the cell debris pelleted. The cell lysate was then used to infect a 15 cm dish containing PER.C6® cells at 80-90% confluence. This infection procedure was continued and expanded at passage 6. The virus was then extracted from the cell pellet by CsCl method. Two bandings were performed (3-gradient CsCl followed by a continuous CsCl gradient). Following the second banding, the virus was dialyzed in A105 buffer. Viral DNA was extracted using pronase treatment followed by phenol chloroform. The viral DNA was then digested with HindIlI and radioactively labeled with [³³P]dATP. Following gel electrophoresis to separate the digestion products the gel was dried down on Whatman paper and then subjected to autoradiography. The digestion products were compared with the digestion products from the pre-plasmid (that had been digested with Pac1/HindIII prior to labeling). The expected sizes were observed, indicating that the virus had been successfully rescued. This strategy was used to rescue virus from each of the various adenovector plasmid constructs prepared.

### EXAMPLE 12

### Stability Analyses

To determine whether the various adenovector constructs (e.g., MRK Ad5 HIV-1 gag) show genetic stability, the viruses were each passaged continually. The viral DNA was analyzed at passages 3, 6 and 10. Each virus maintained its correct genetic structure. In addition, the stability of the MRK Ad5 HIV-1 gag was analyzed under propagation conditions similar to that performed in large scale production. For this analysis, the transfections of MRK Ad5 HIV-1 gag as well as three other adenoviral vectors were repeated and the virus was purified at P3. The three other adenovectors were as follows: (1) that comprising hCMV(no intron)-F1gag with a bGHpA terminator in an E3−adenovector backbone; (2) that comprising hCMV(no intron)-F1gag with a SPA termination signal in an E3+adenovector backbone, and that comprising a mCMV-F1gag with a bGHpA terminator in an E3+adenovector backbone. All of the vectors have the transgene inserted in the E1 parallel orientation. Viral DNA was analyzed by radioactive restriction analysis to confirm that it was correct before being delivered to fermentation cell culture for continued passaging in serum-free media. At P5 each of the four viruses were purified and the viral DNA extracted for analysis by the restriction digestion and radiolabeling procedure. This virus has subsequently been used in a series of studies (in vitro gag expression in COS cells, rodent study and rhesus monkey study) as will be described below. The viruses from P5 are shown in FIG. 9.

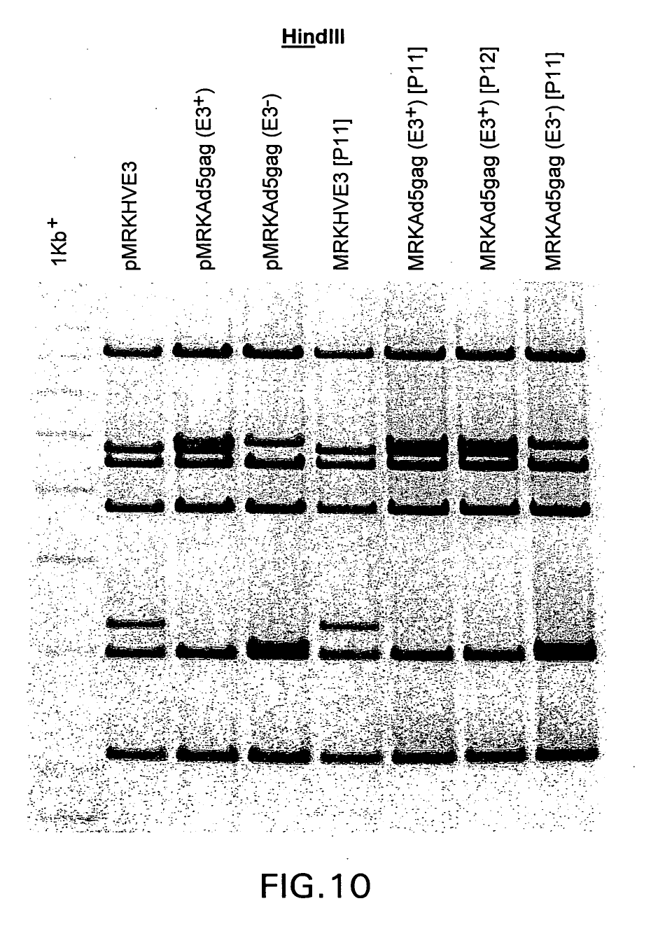

The passaging under serum-free conditions was continued for the MRKHVE3 (transgene-less, obtained from MRKpAdHVE3 pre-plasmid) and the MRKAd5HIV-1gag (obtained from MRKpAdHVE3+CMV(no intron)-FLgag-bGHpA pre-plasmid) viruses. FIG. 10 shows viral DNA analysis by radioactive restriction digestion at passage 11 for MRKHVE3, MRKAd5HIV-1gagE3-, and passage 11 and 12 for MRKAd5HIV-1gag. Aside from the first lane which is the DNA marker lane, the next three lanes are virus from the pre-plasmid controls (controls based on the original virus)—MRKpAdHVE3 (also referred to as “pMRKHVE3”), MRKpAdHVE3+CMV(no intron)-FLgag-bGHpA, and pMRKAd5gag(E3−), respectively. As seen in FIG. 10, each of the viral DNA samples show the expected bands with no extraneous bands showing. This signifies that there are no major variant adenovirus species present that can be detected by autoradiography.

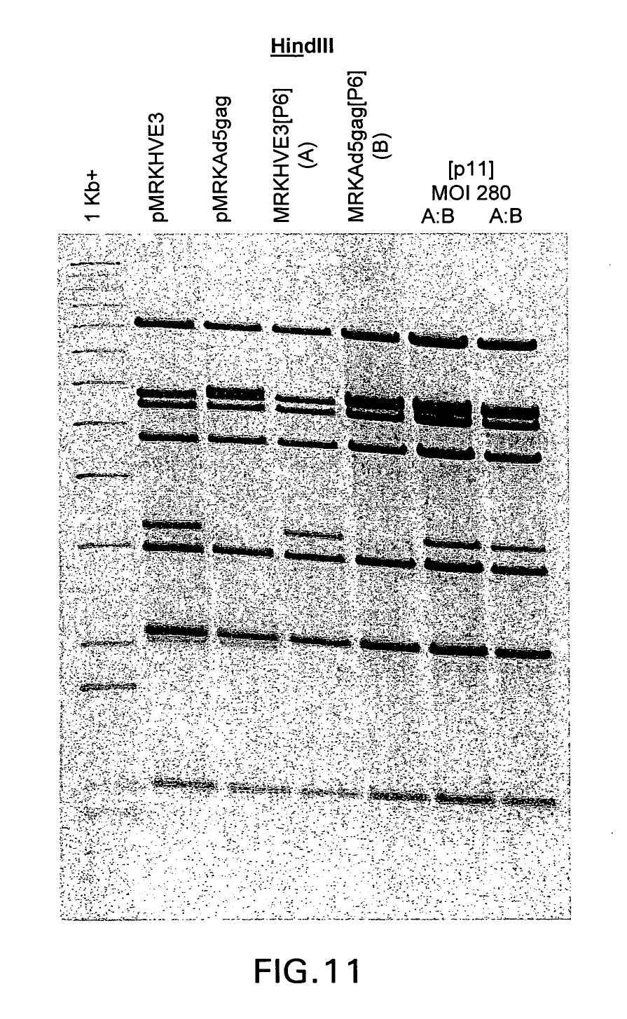

FIG. 11 shows the results of viral competition study between MRKHVE3 and MRKAd5HIV-1gag. These viruses were mixed together at equal MOI (140 viral particles each; 280 vp total) at passage 6 and continued to be passaged until P11. Aside from the first lane which is the DNA marker lane, the next two lanes are the pre-plasmid controls obtained from MRKpAdHVE3 and MRKpAdHVE3+CMV(no intron)-FLgag-bGHpA. The next two lanes are the viral DNA from the starting viral material at passage six. The last two lanes are the competition studies performed in duplicate. The data in FIG. 11 shows the effect the gag transgene in culture. Growth of a MRKAd5gag virus was compared with growth of a “transgene-less” MRKHVE3. These two viruses were infected at the same MOI (i.e. 140 vp each) at passage 6 and then passaged through to passage 11 and the viral pool was analyzed by radioactive restriction analysis. The data shows that one virus did not out compete the other. Therefore, the gag transgene did not show obvious signs of toxicity to the adenovirus.

Analysis by Hind/III digestion shows that each virus specie is present in approximately equal amounts. As above, there does not appear to be signs of any extraneous bands. FIG. 12 shows higher passage numbers for MRKAd5HIV-1gag grown under serum-containing conditions. The genome integrity again has been maintained and there is no evidence of rearrangements, even at the highest passage level (P21).

Each of the four vectors shown in FIG. 9 were analyzed for amplification capacity. Table 4 below shows the QPA analysis used in the estimation of viral amplification ratios at P4. The determination of the amplification ratio for the original HIV-1 gag construct is based on the clinical lot at P12. It has been shown that amplification rates increases with higher passage number for the original virus. The reason for this observation is due to the emergence of variants which exhibit increased growth rates compared to the intact adenovector. With continued passaging of the original Ad gag vector, the level of variants increases and hence amplification rates increase also.

The MRK Ad5 HIV-1 gag virus has also been continually passaged under process conditions (i.e., serum-free media). Viral DNA extracted from passages 11 and 12 show no evidence of rearrangement.

TABLE 4
Amplification Ratios Based on AEX and QPA Analysis of
Virus Amplification from Passage 3 to Passage 4.

| Ad gag construct | Amplification Ratio |
|---|---|
| MRKAd5gag | 470 |
| HCMV-Flgag-bGHpA [E3−] | 115 |
| HCMV-Flgag-SPA [E3+] | 320 |
| mCMV-FLgag-bGHpA [E3+] | 420 |
| Original construct* | 40-50 |

*This estimation is based on the clinical lot growth characteristics at Passage 12.

### EXAMPLE 13

### Analytical Evaluation of the Enhanced Ad5 Constructs

To study the effects of the transgene and the E3 gene on virus amplification, the enhanced adenoviral vector, MRK Ad5 HIV-1 gag, along with its transgene-less version (MRKpAdHVE3) and its E3−version (MRK Ad5 HIV-1 gag E3−), was studied for several passages under serum-free conditions. Table 5A shows the amplification ratios determined for passages P3 to P8 for MRK Ad5 HIV-1 gag. Within a certain MOI range, it has been determined that the virus output is directly proportional to the virus input. Therefore, the greater the number of virus particles per cell at infection, the greater the virus amount produced. Viral amplification ratios, on the other hand, are inversely proportional to the virus input. The lower the virus input, the greater the amplification ratio.

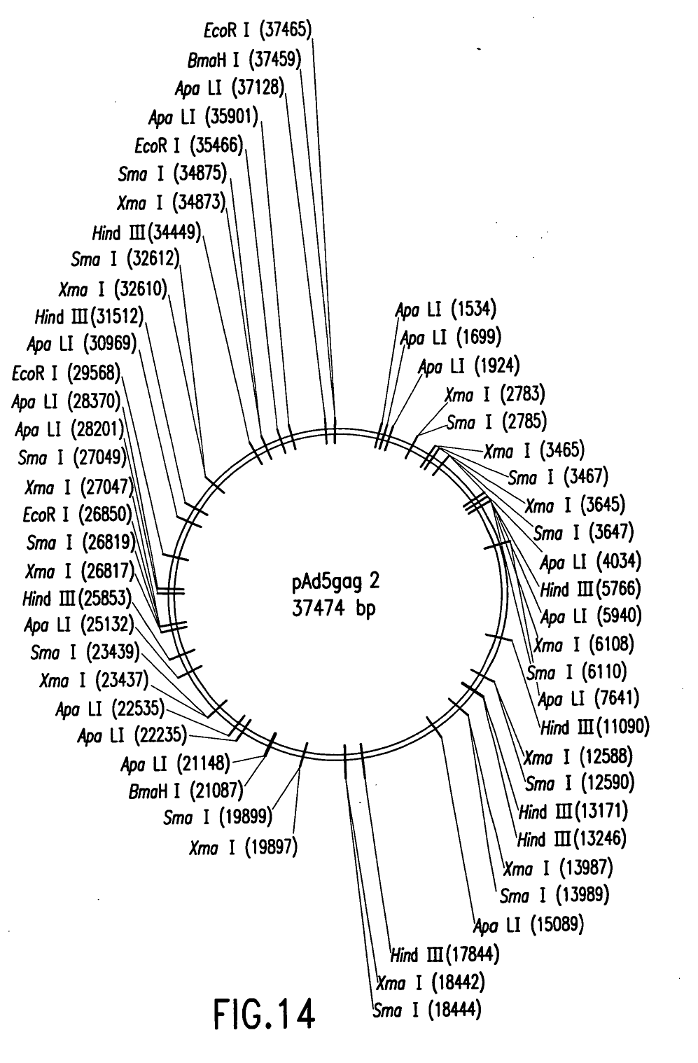

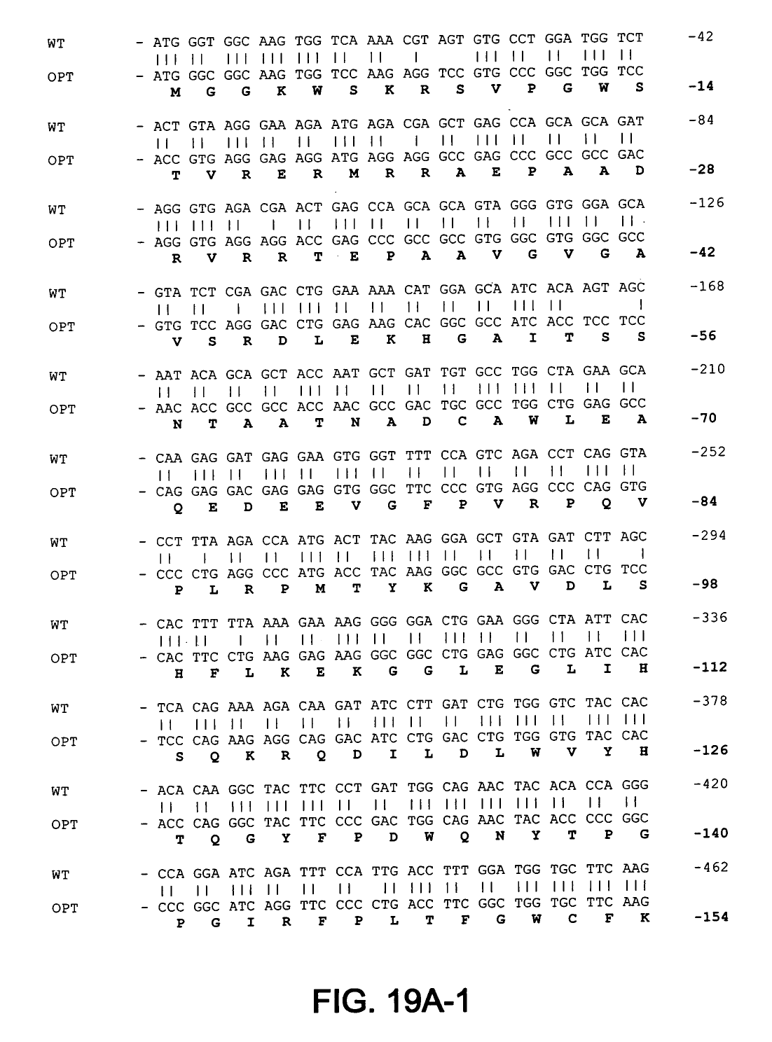

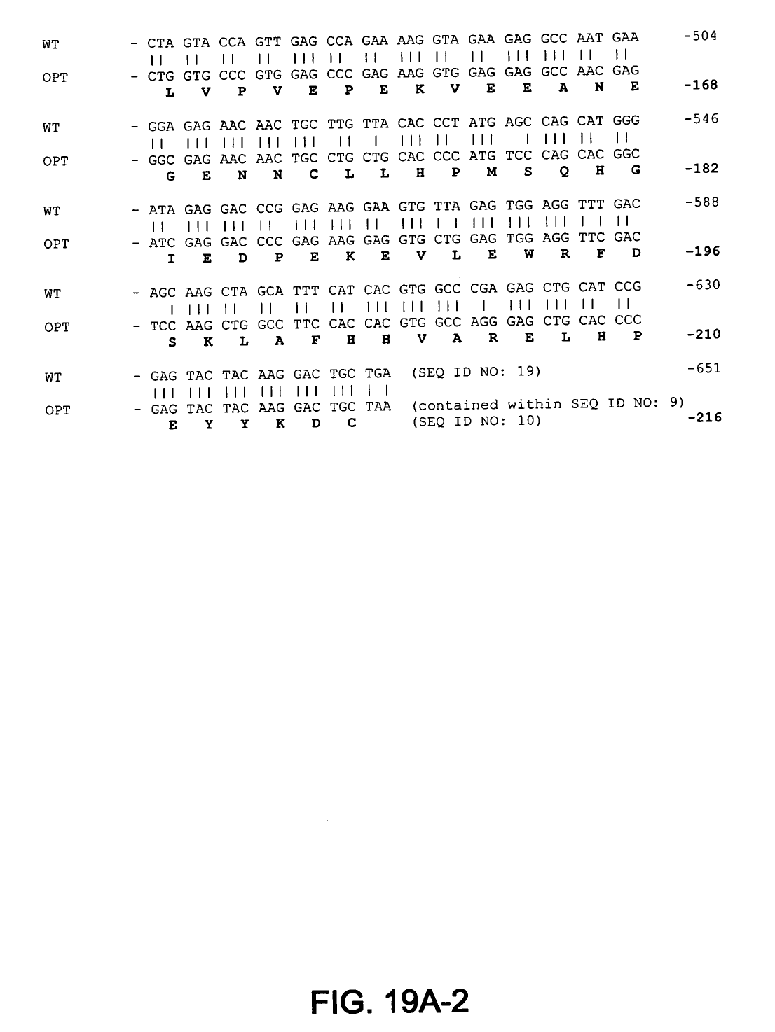

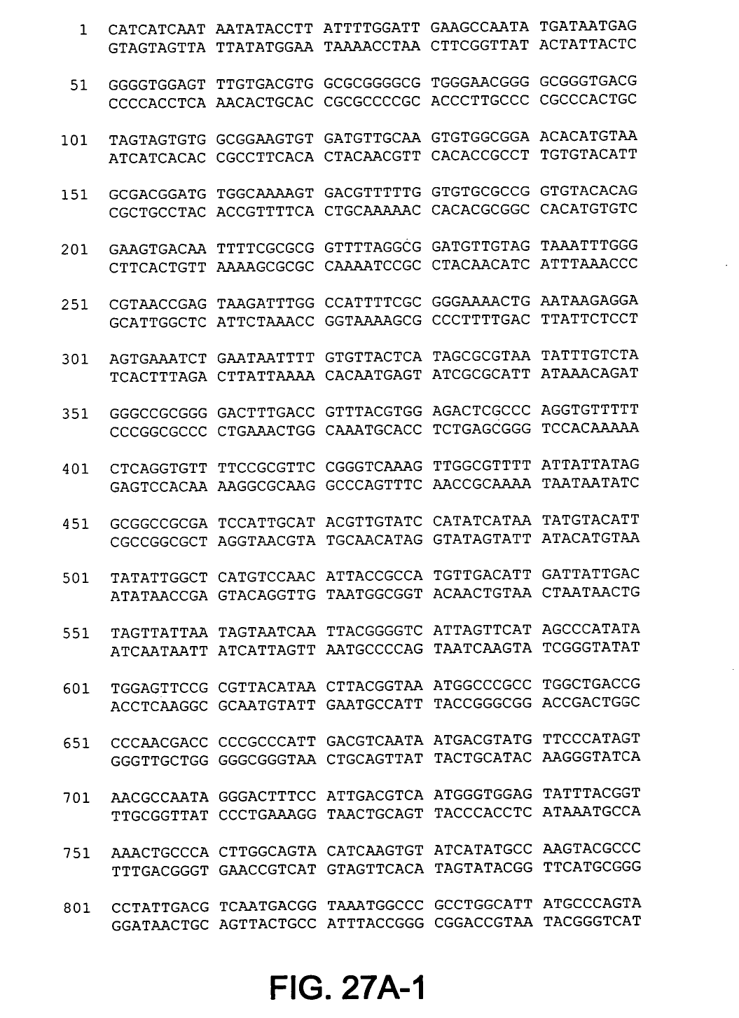

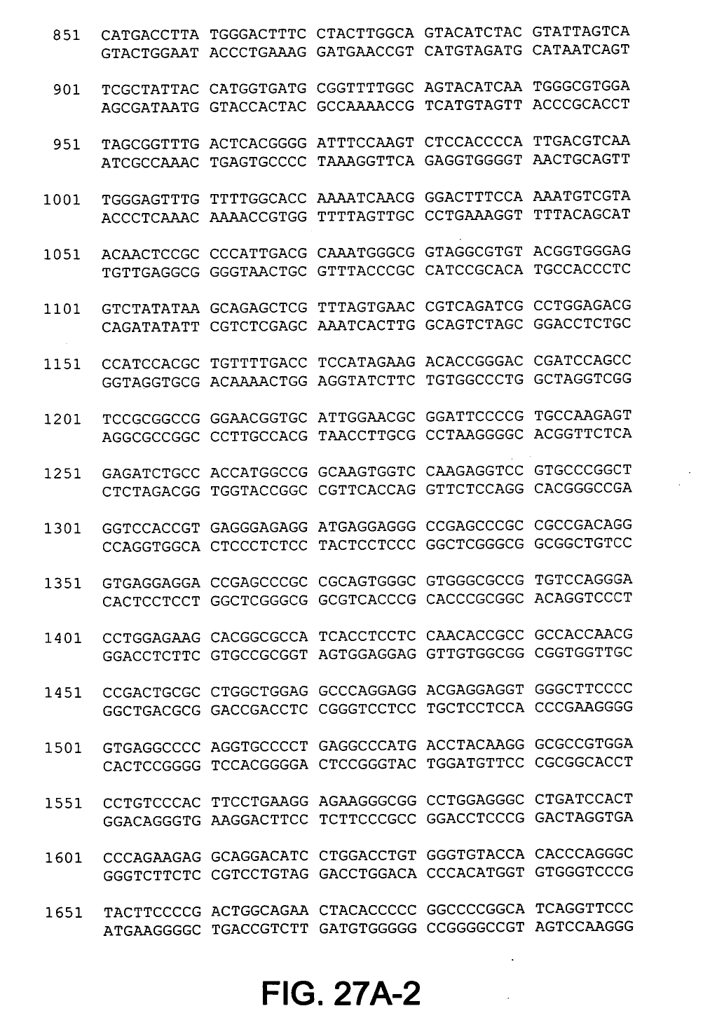

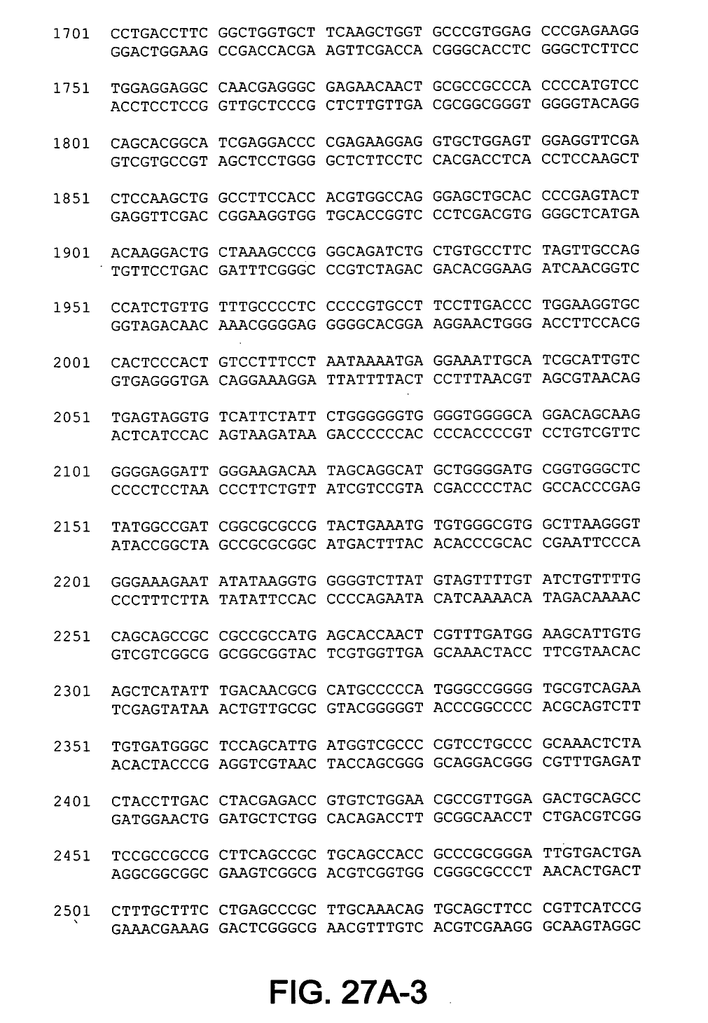

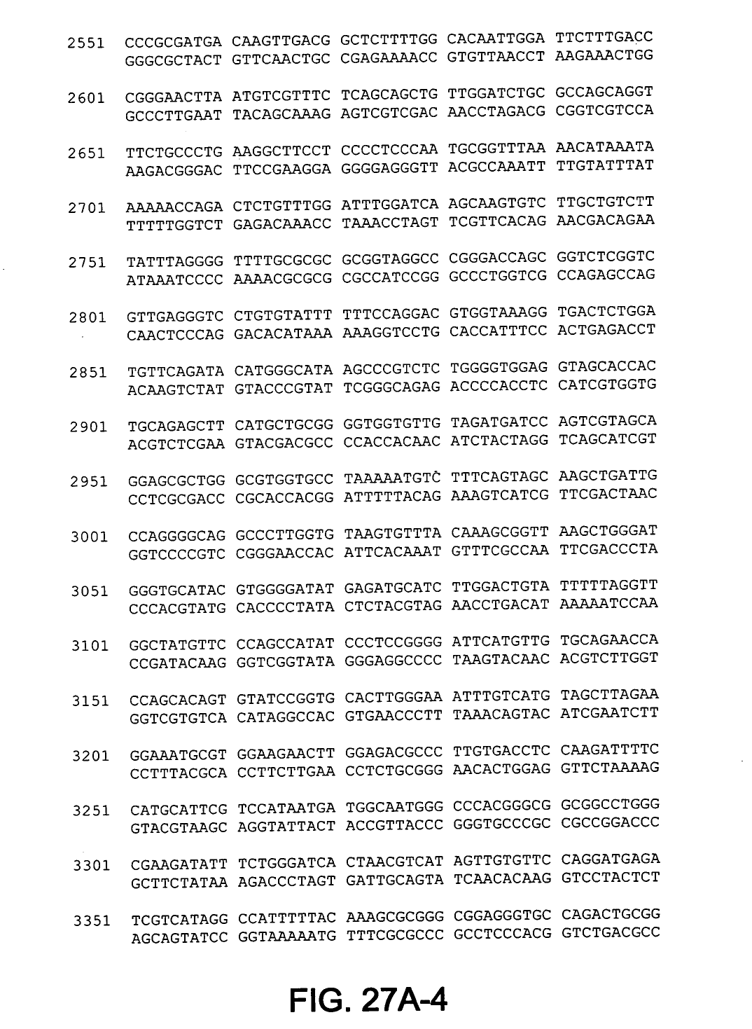

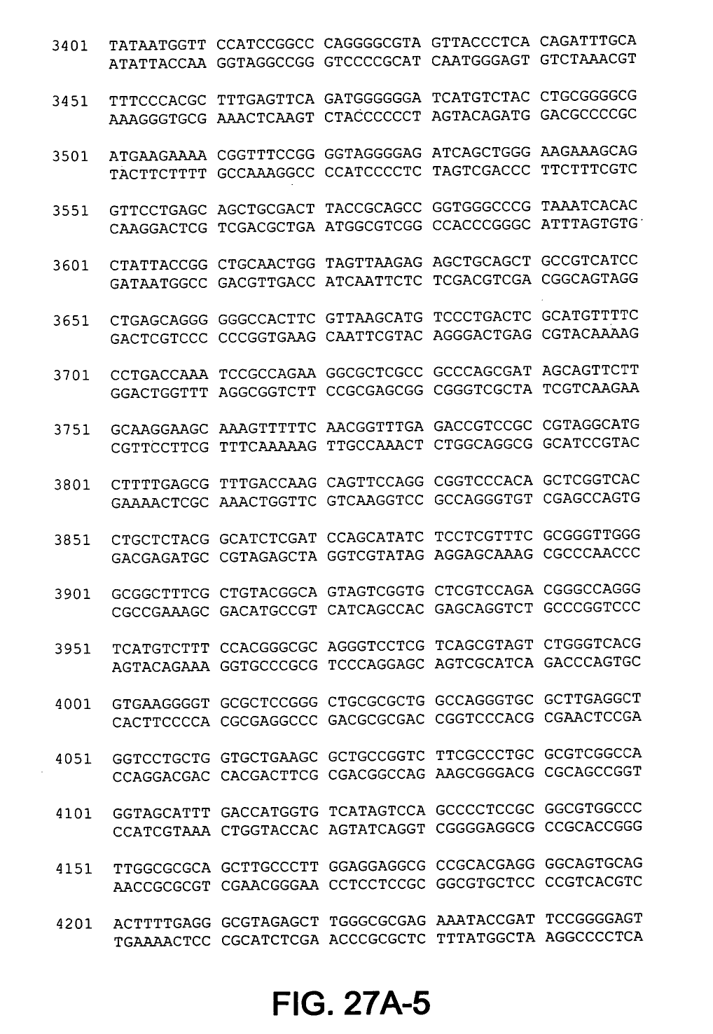

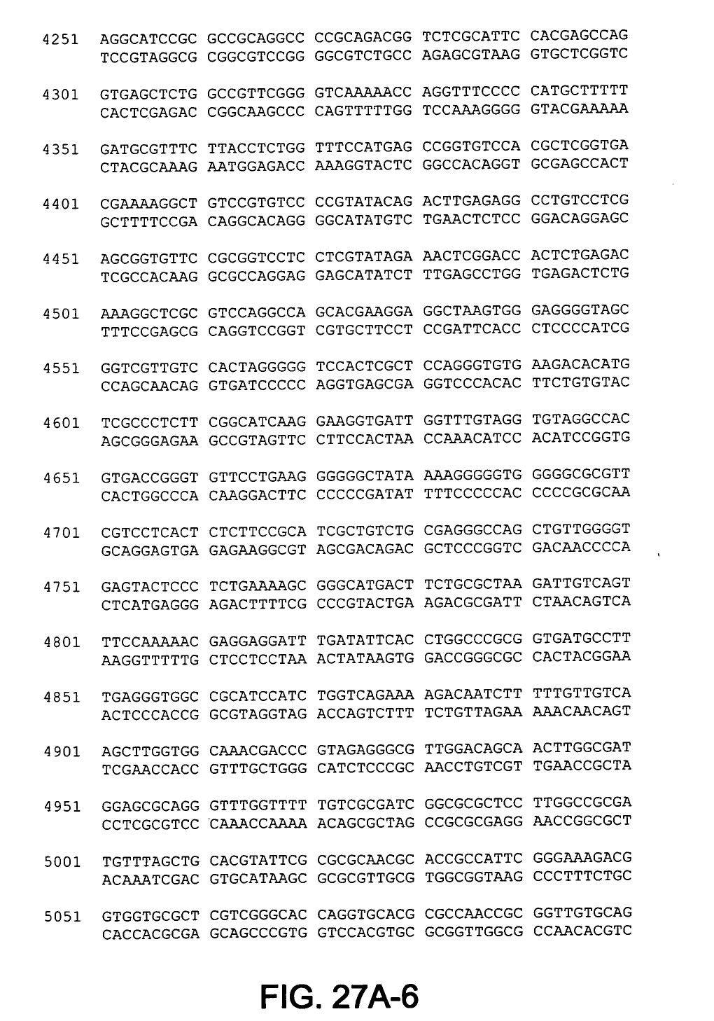

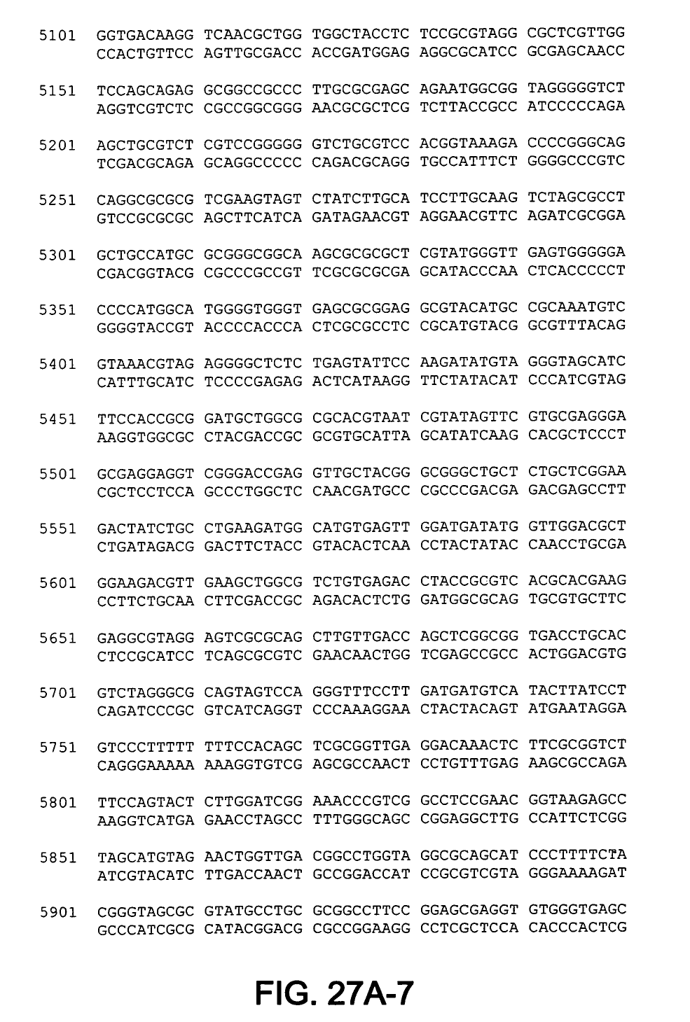

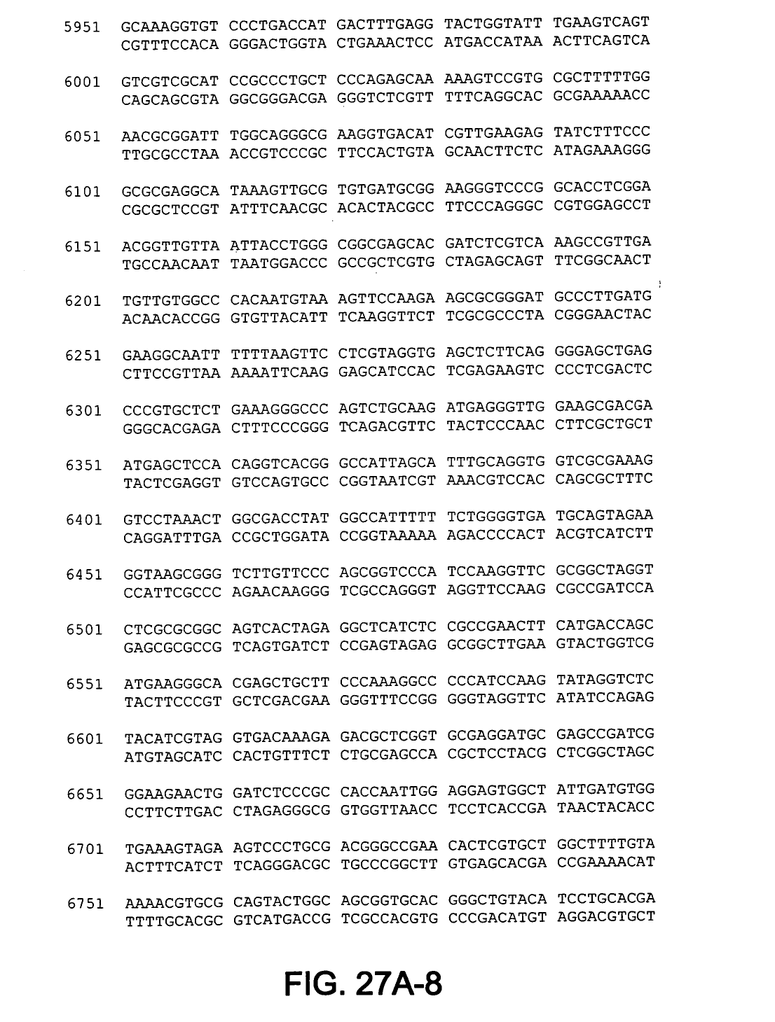

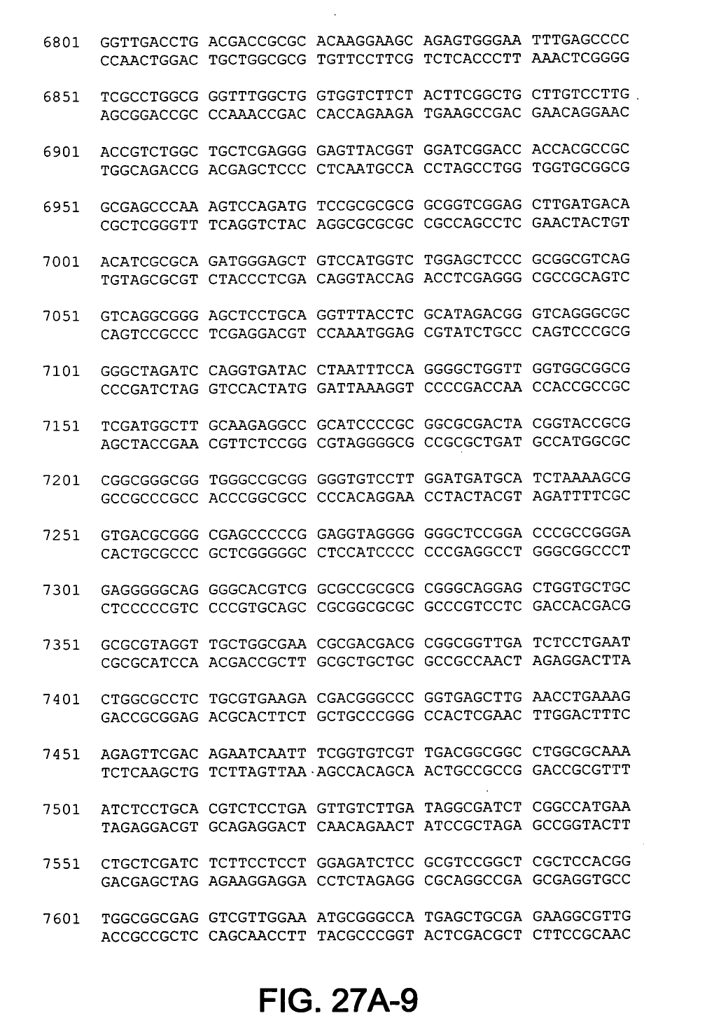

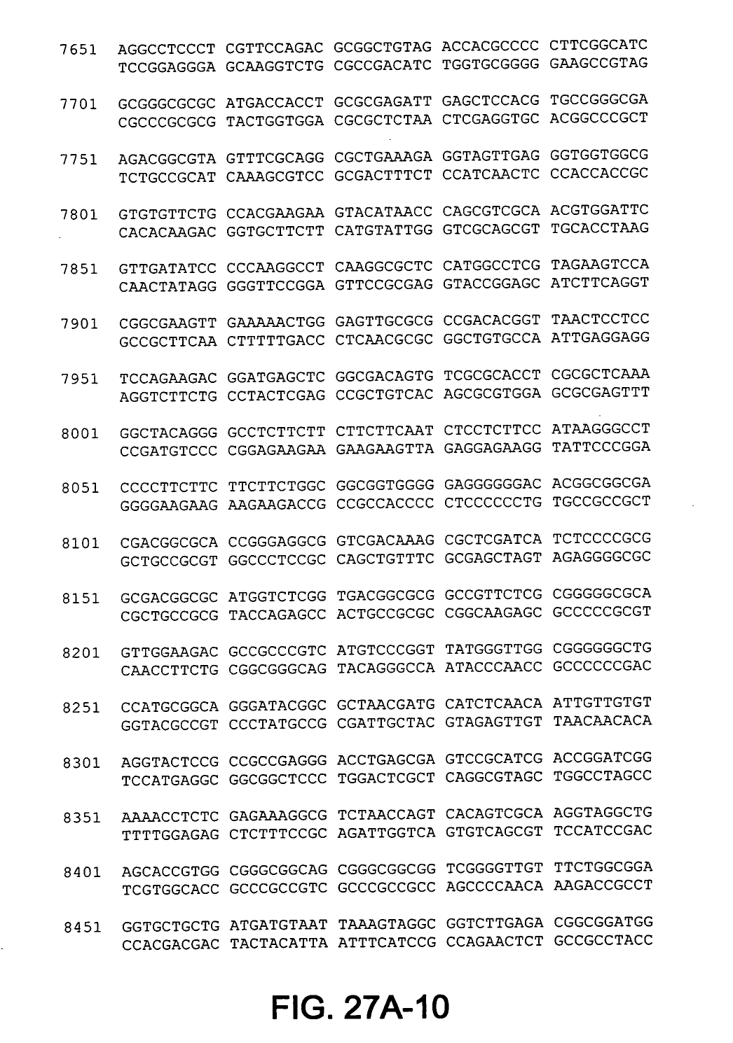

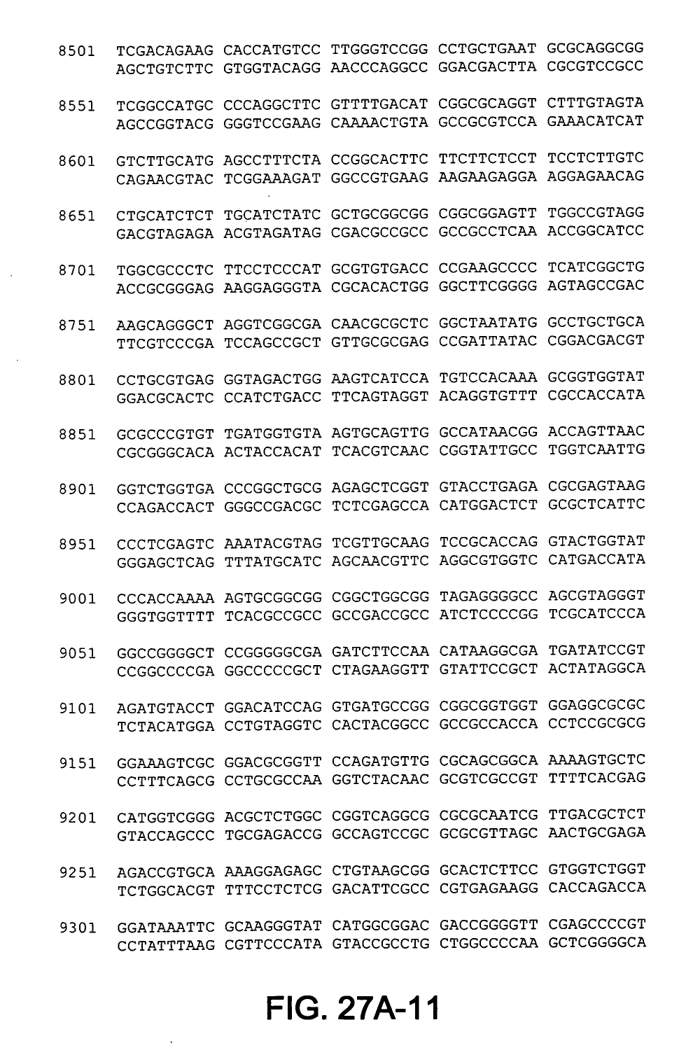

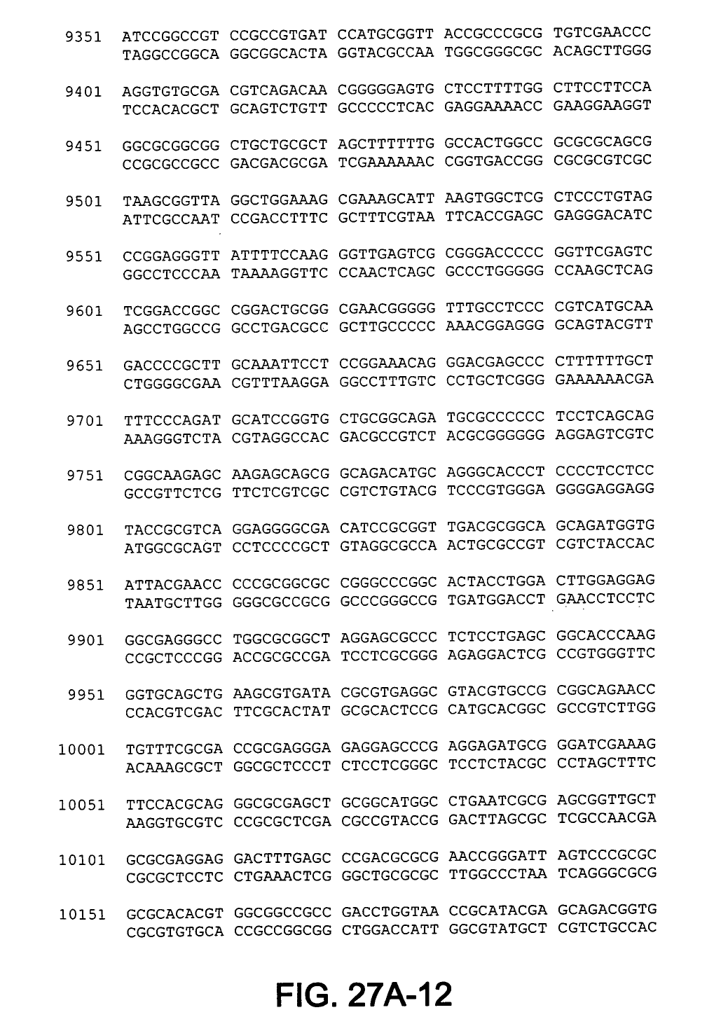

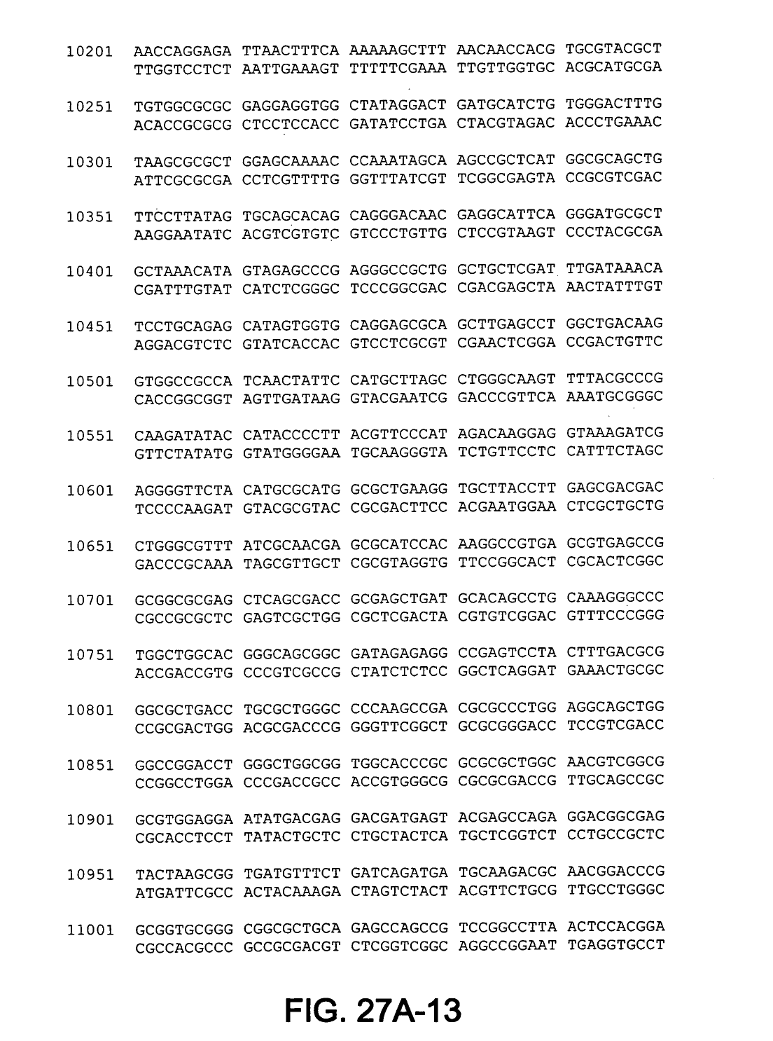

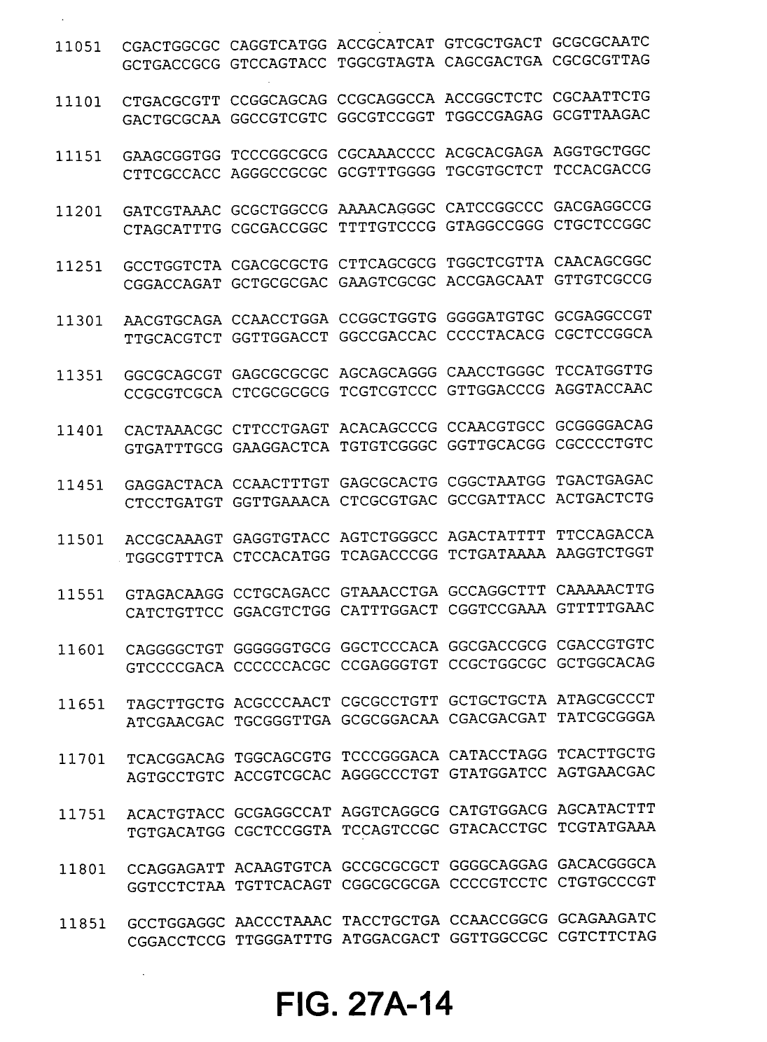

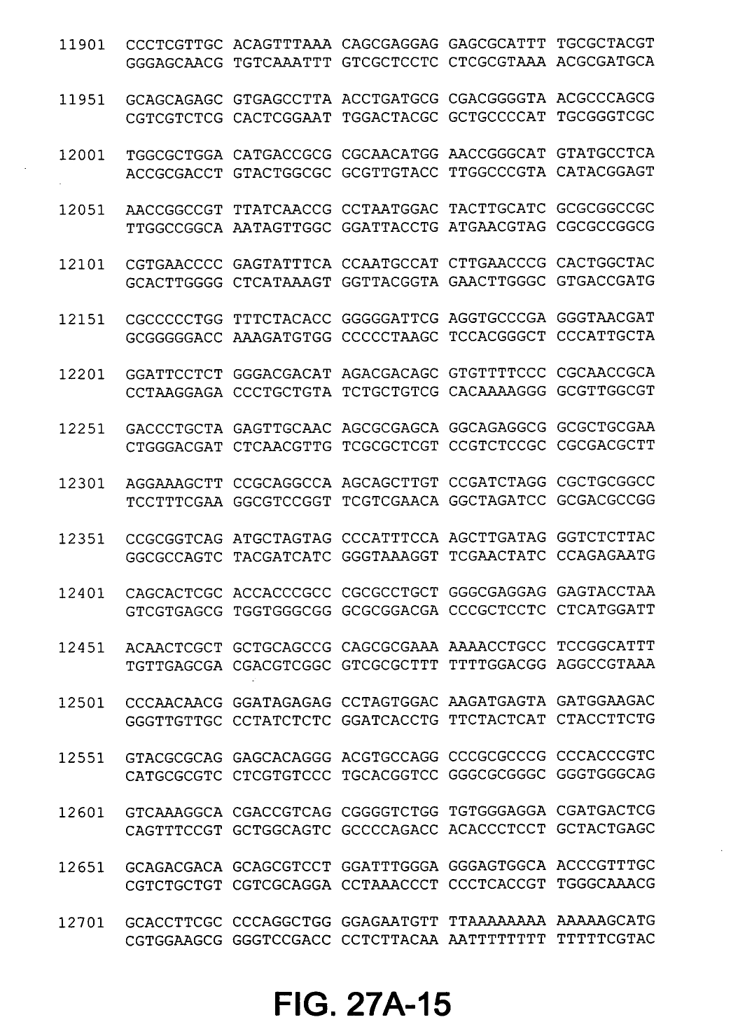

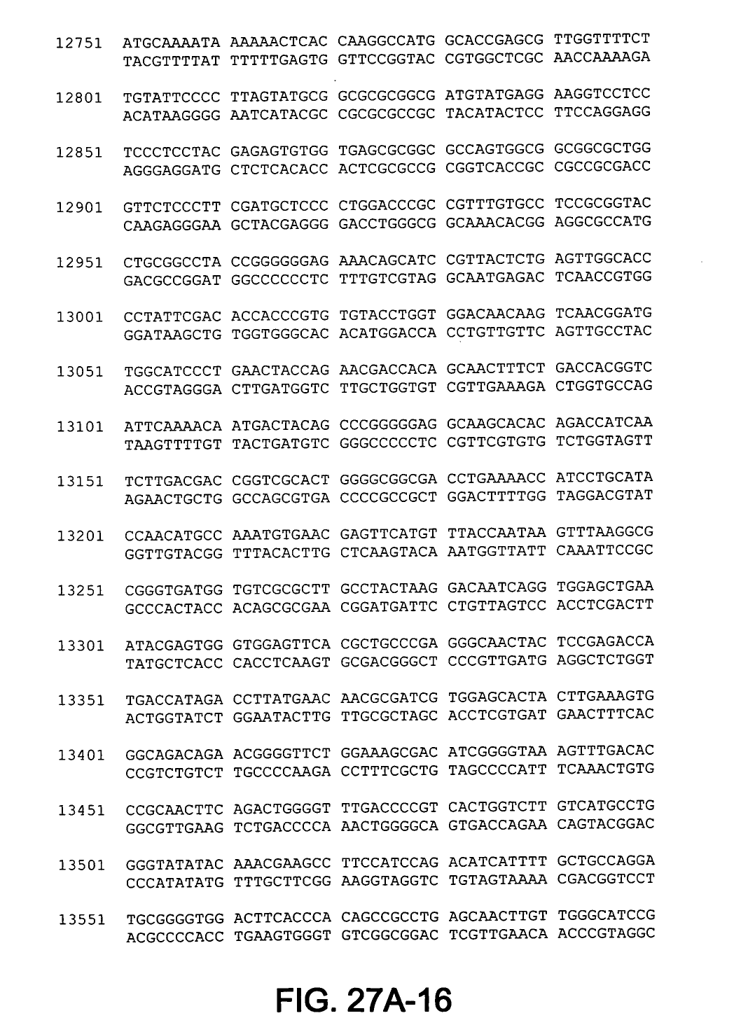

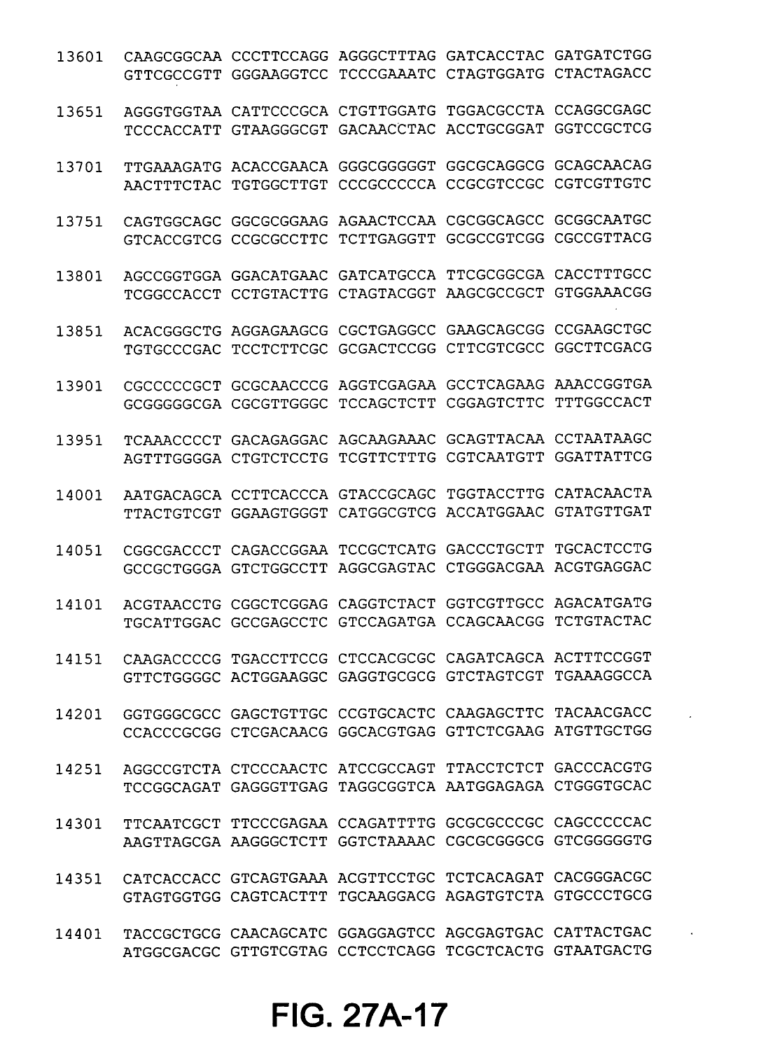

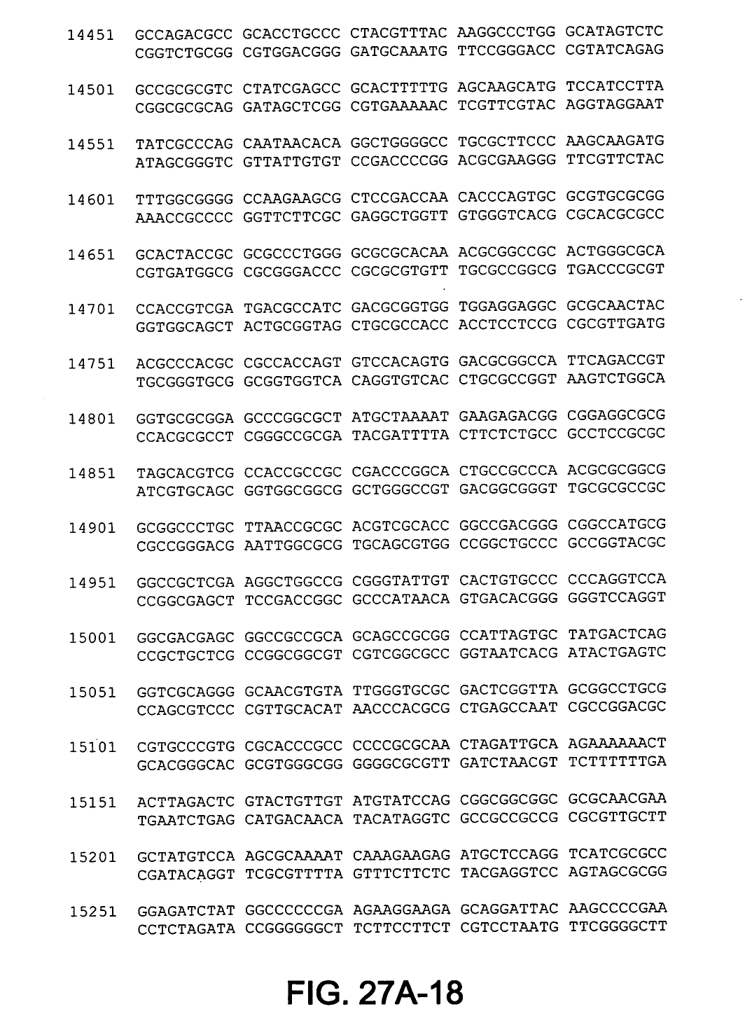

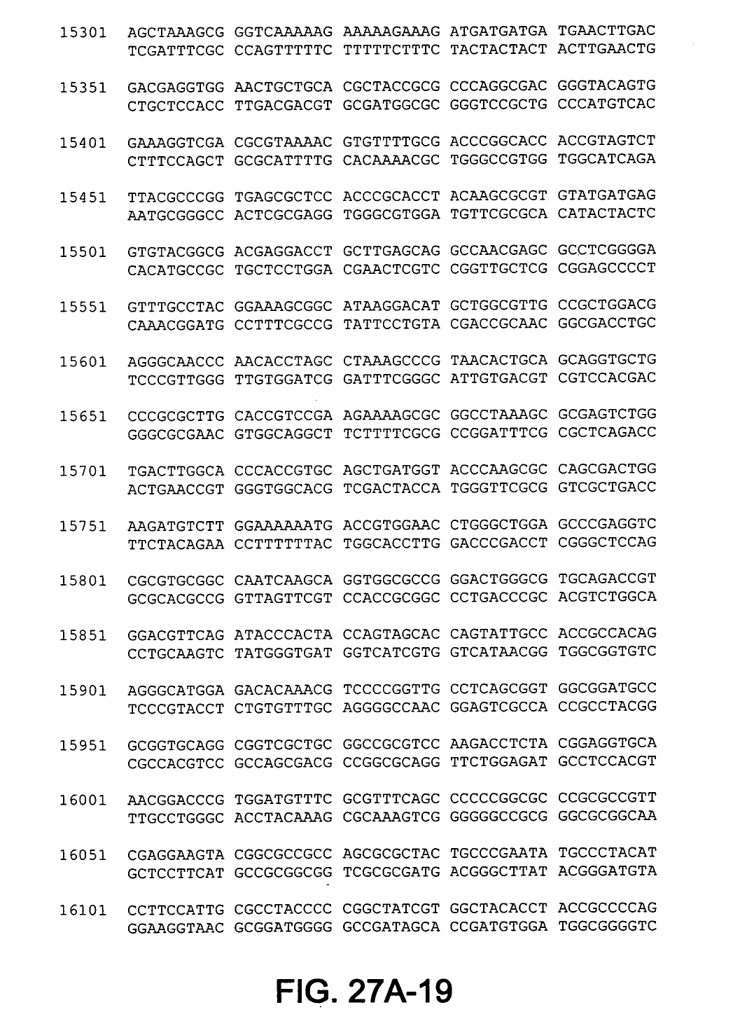

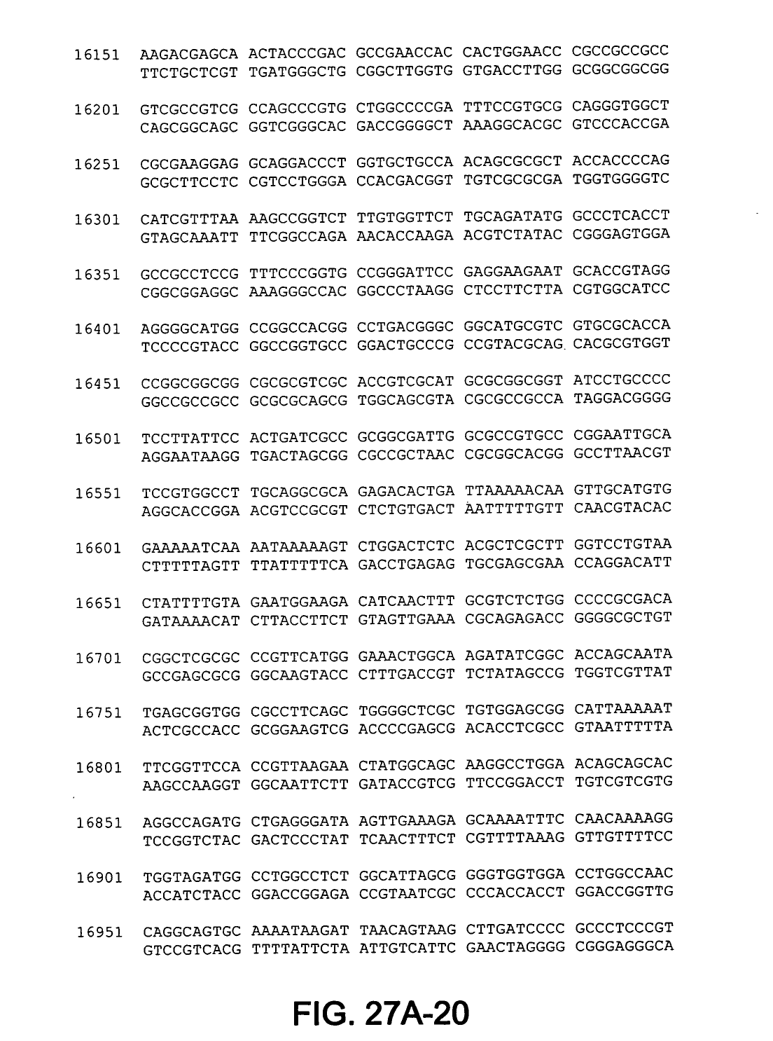

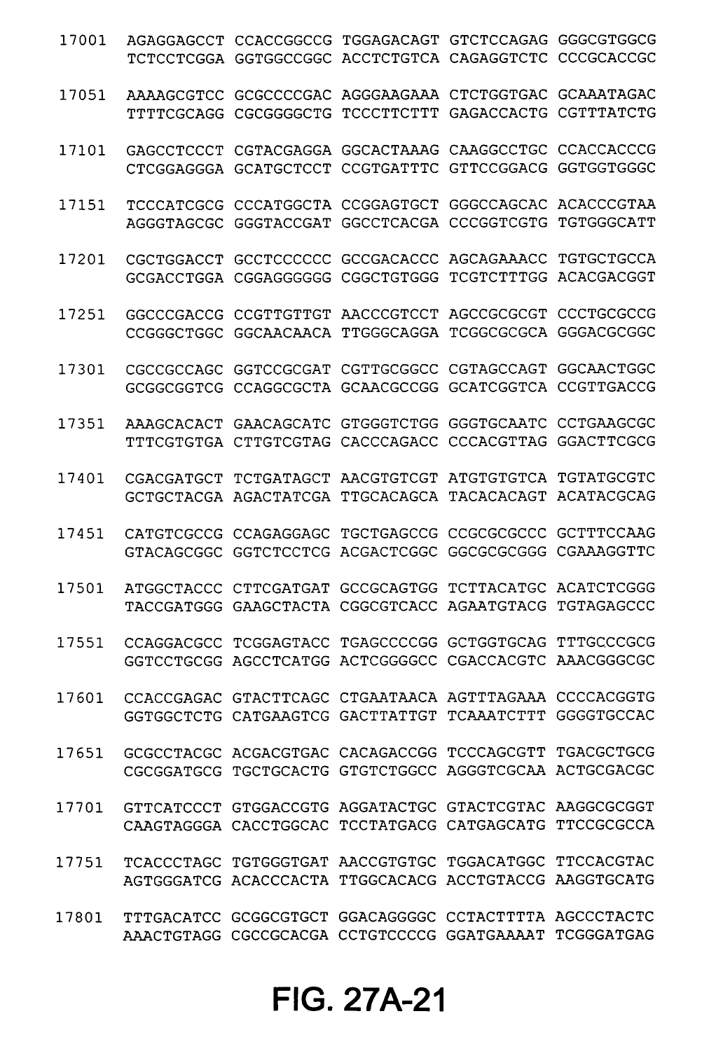

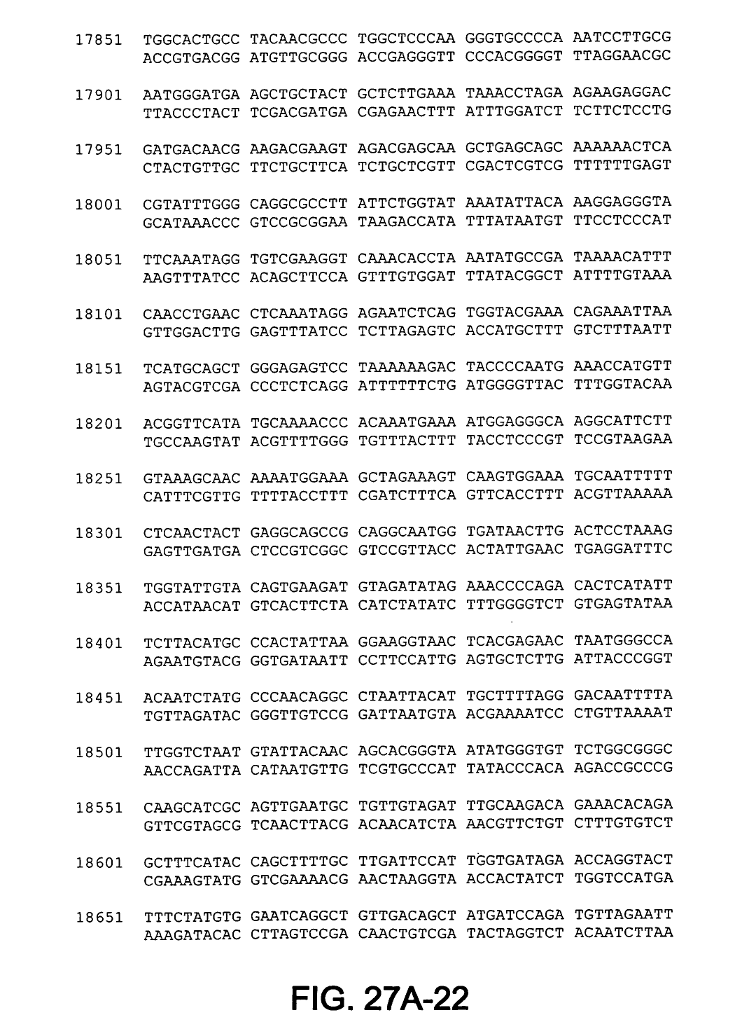

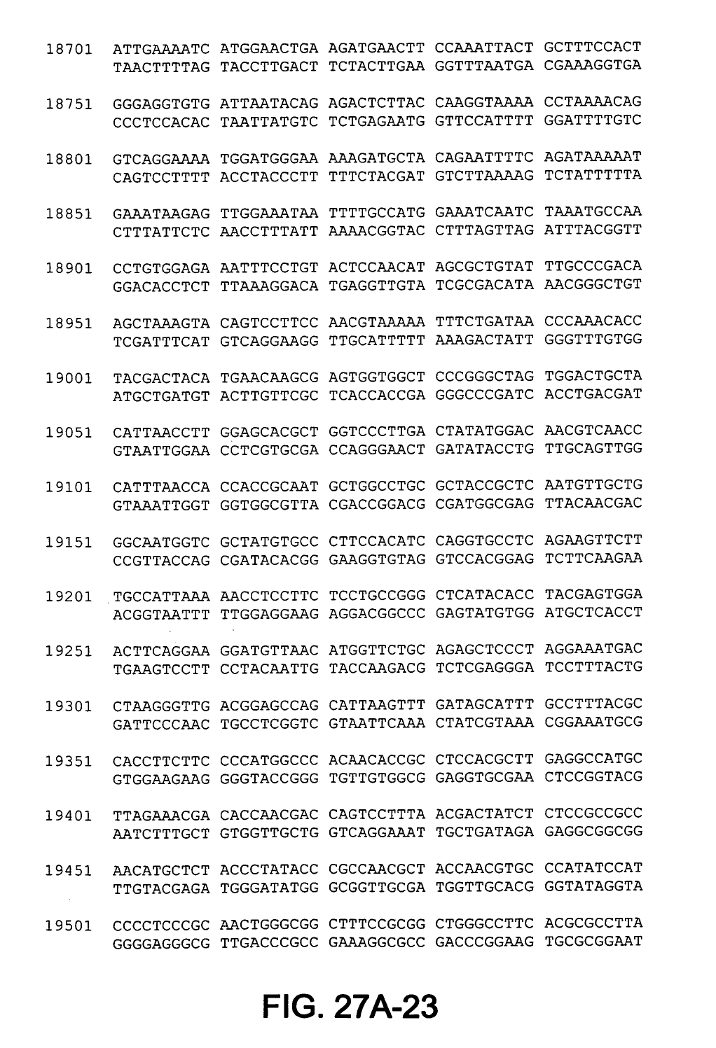

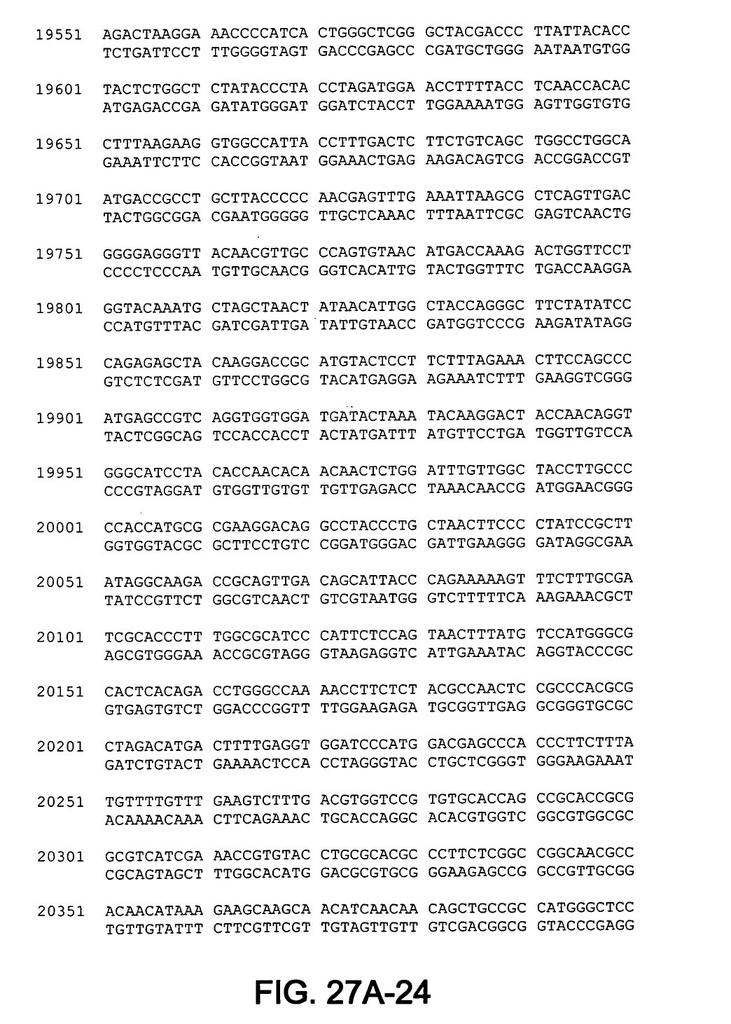

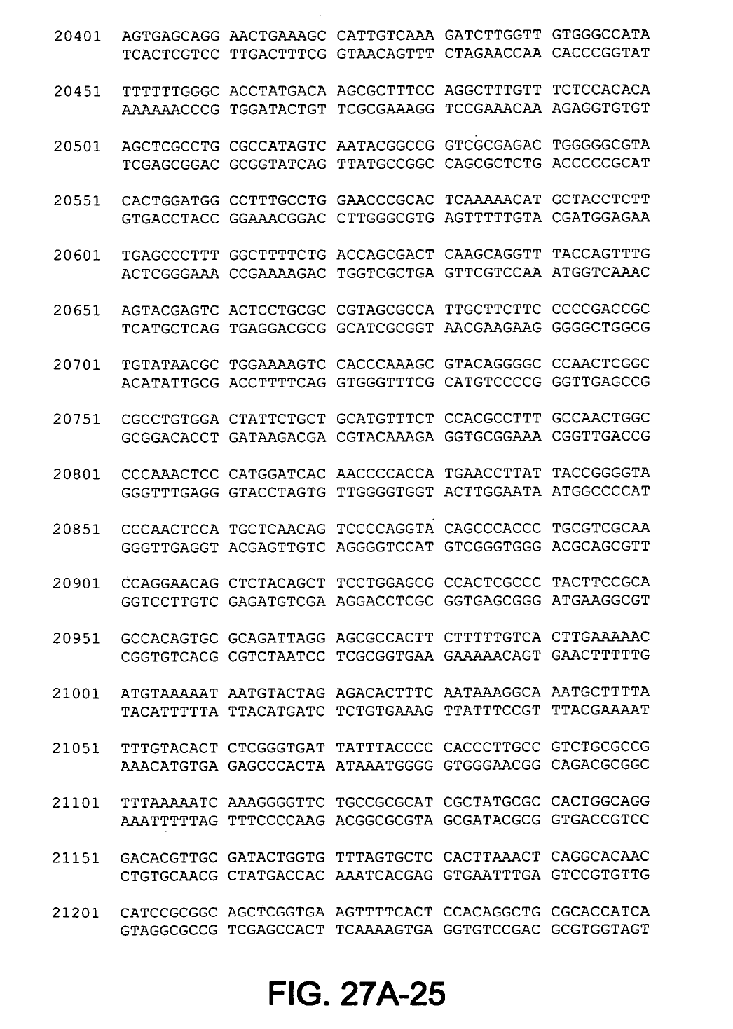

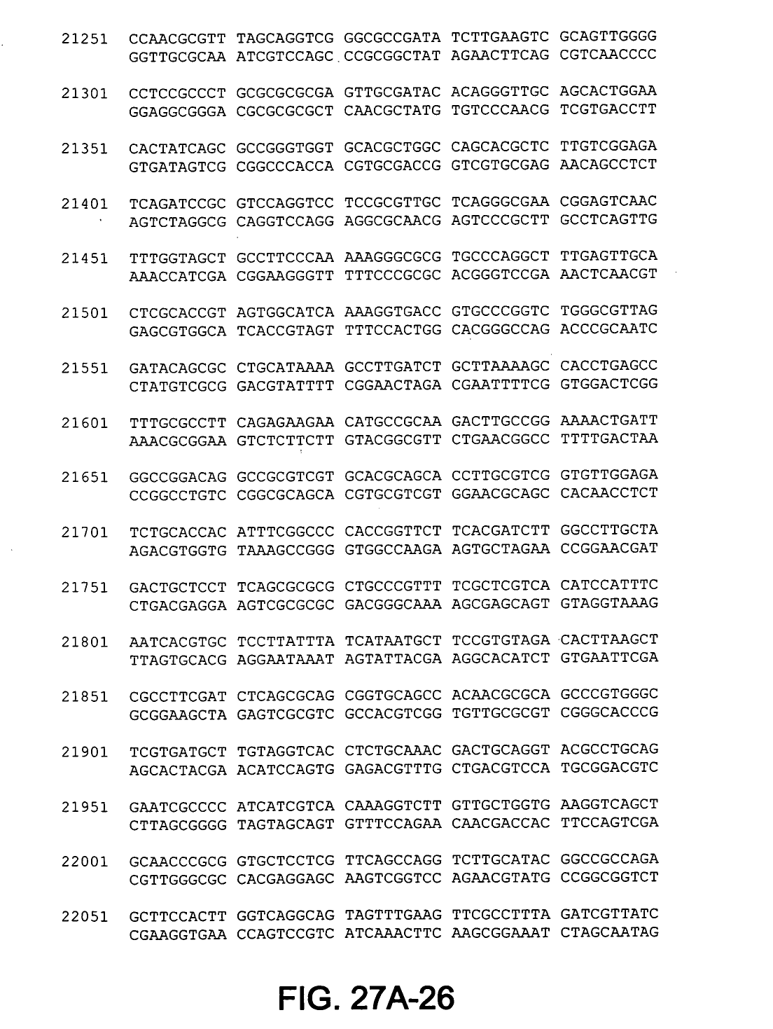

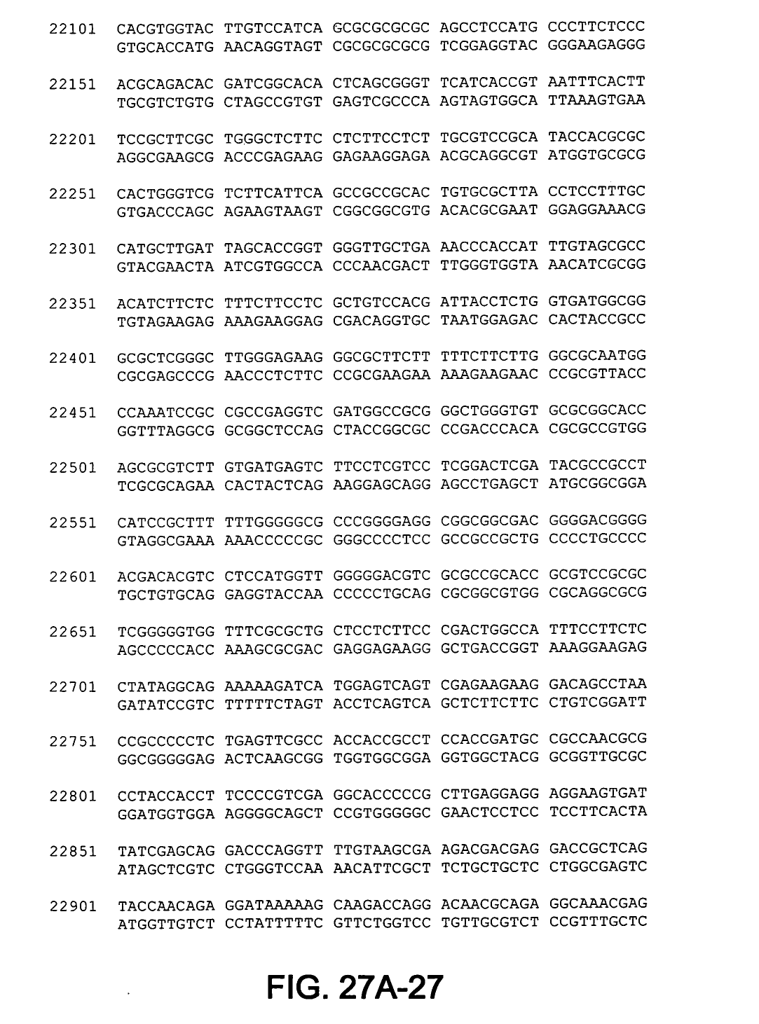

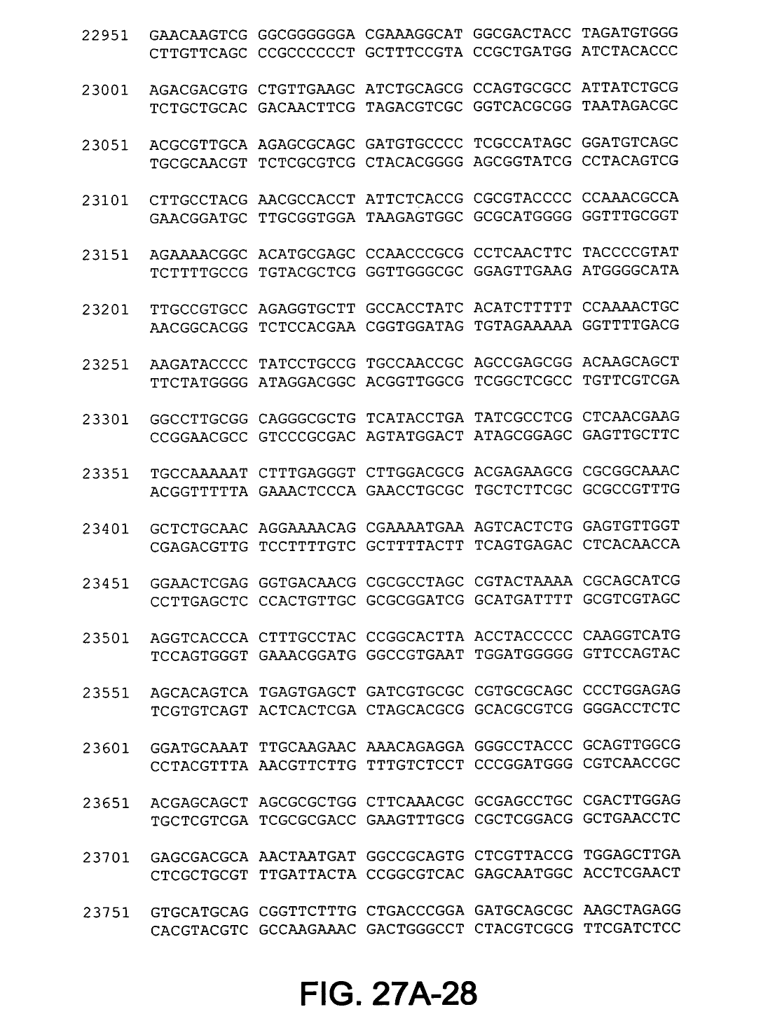

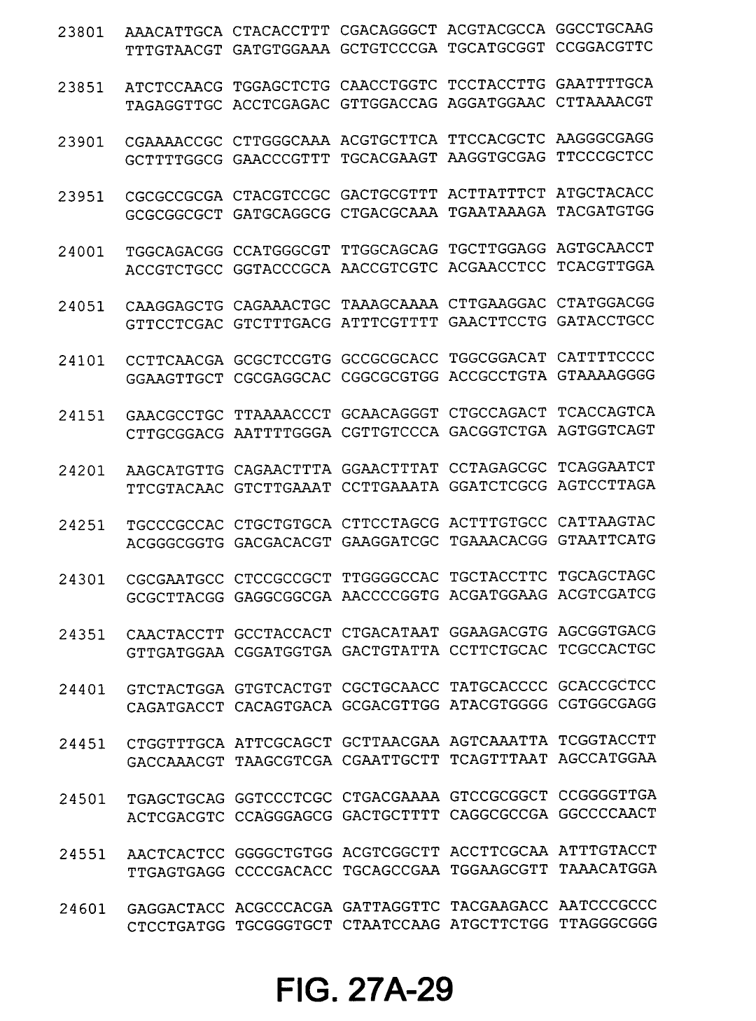

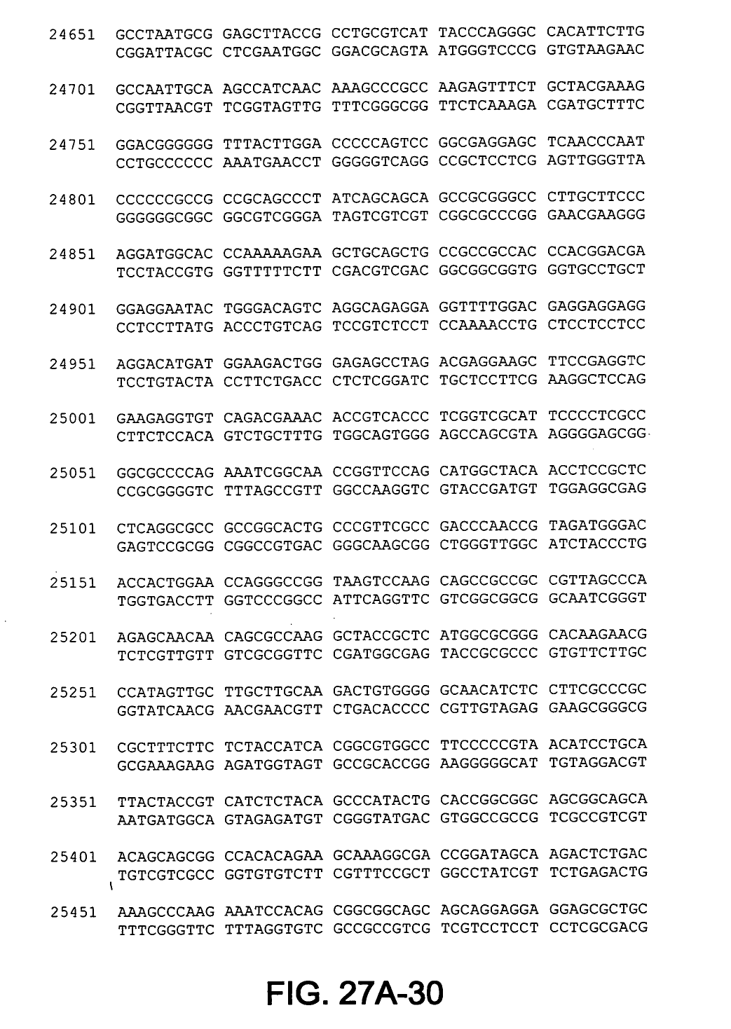

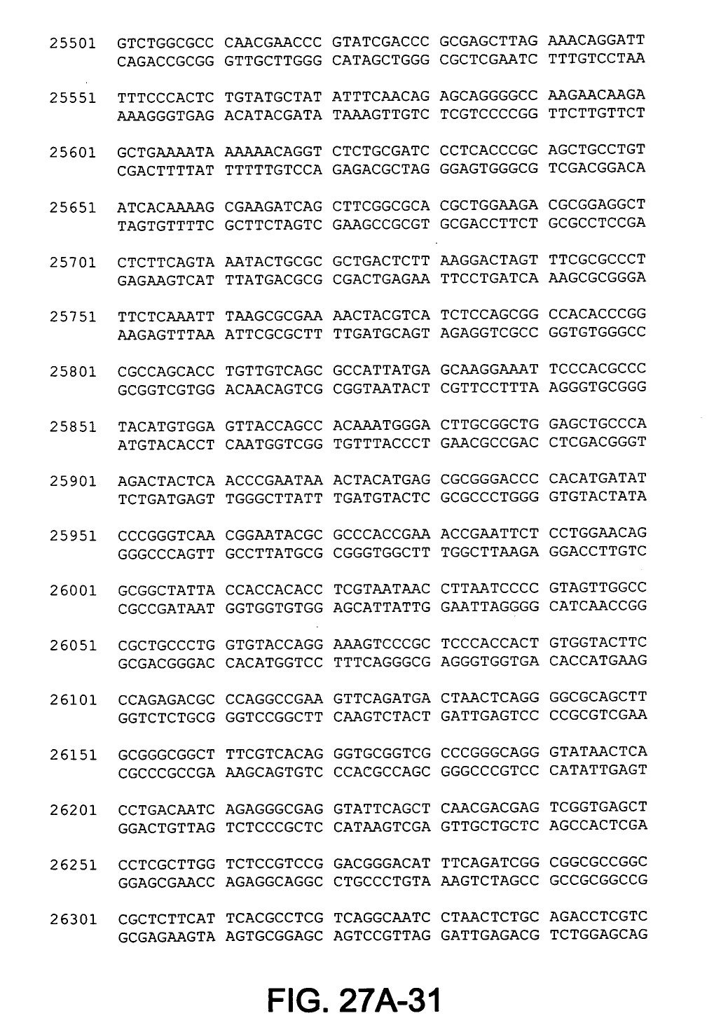

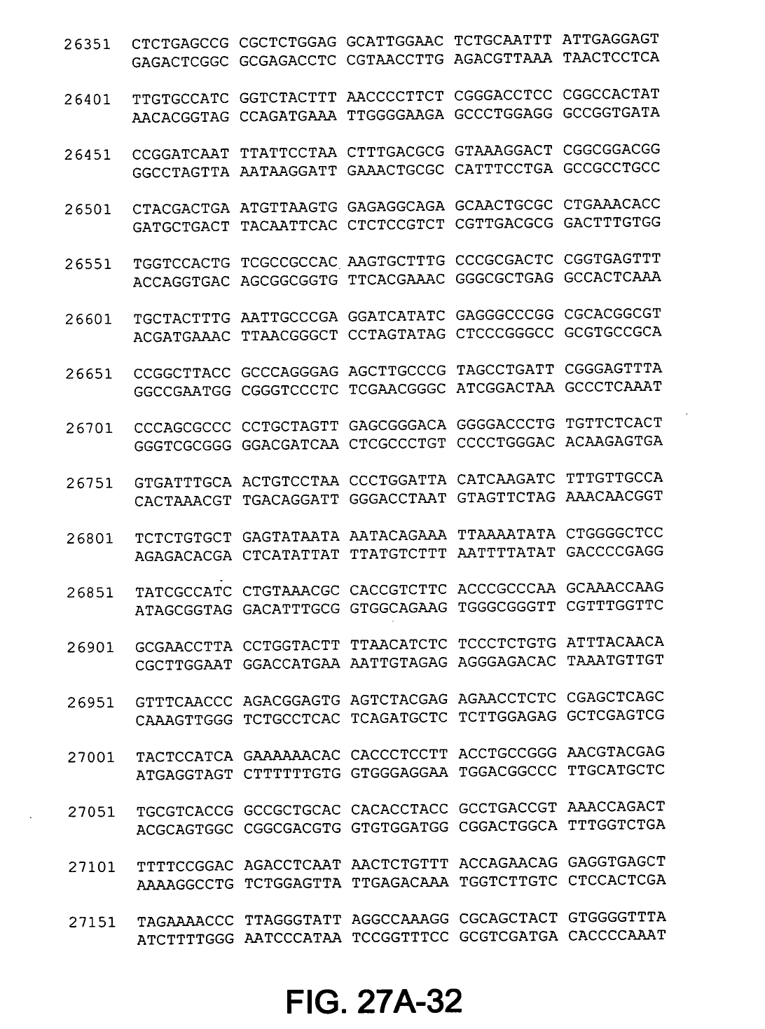

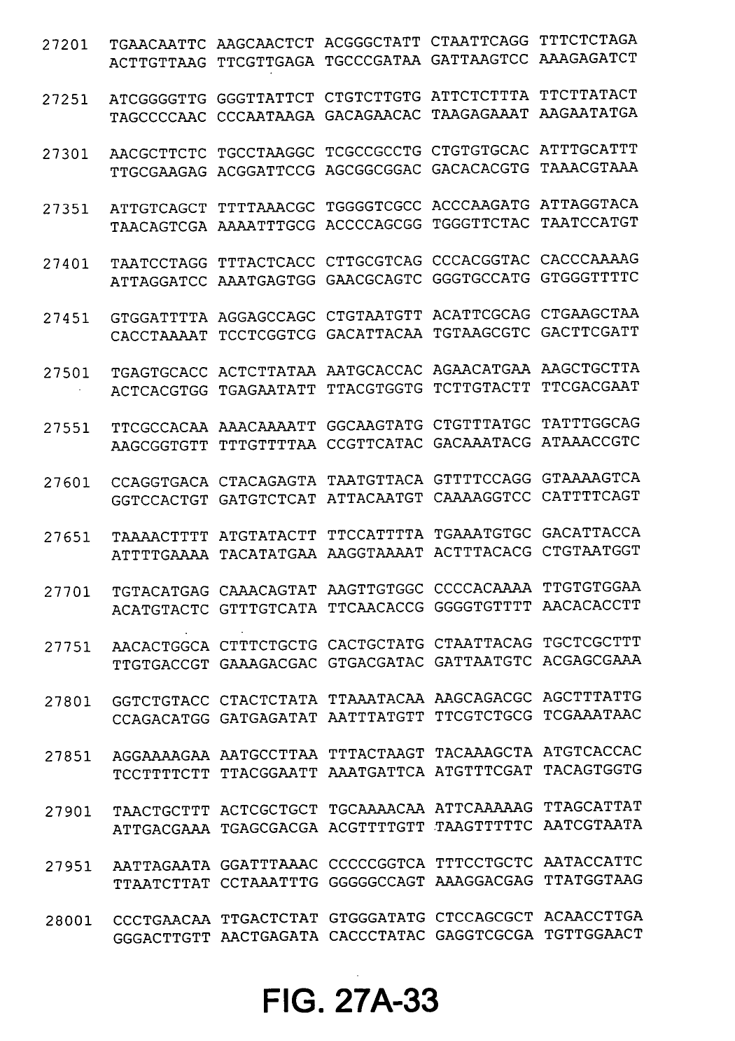

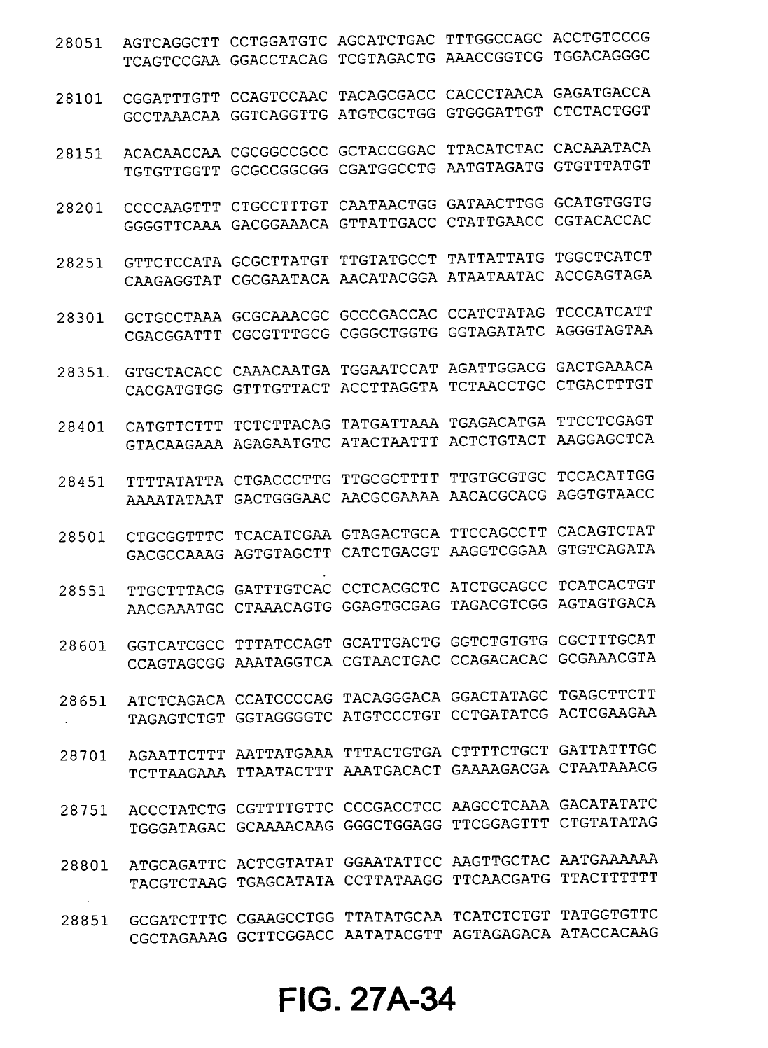

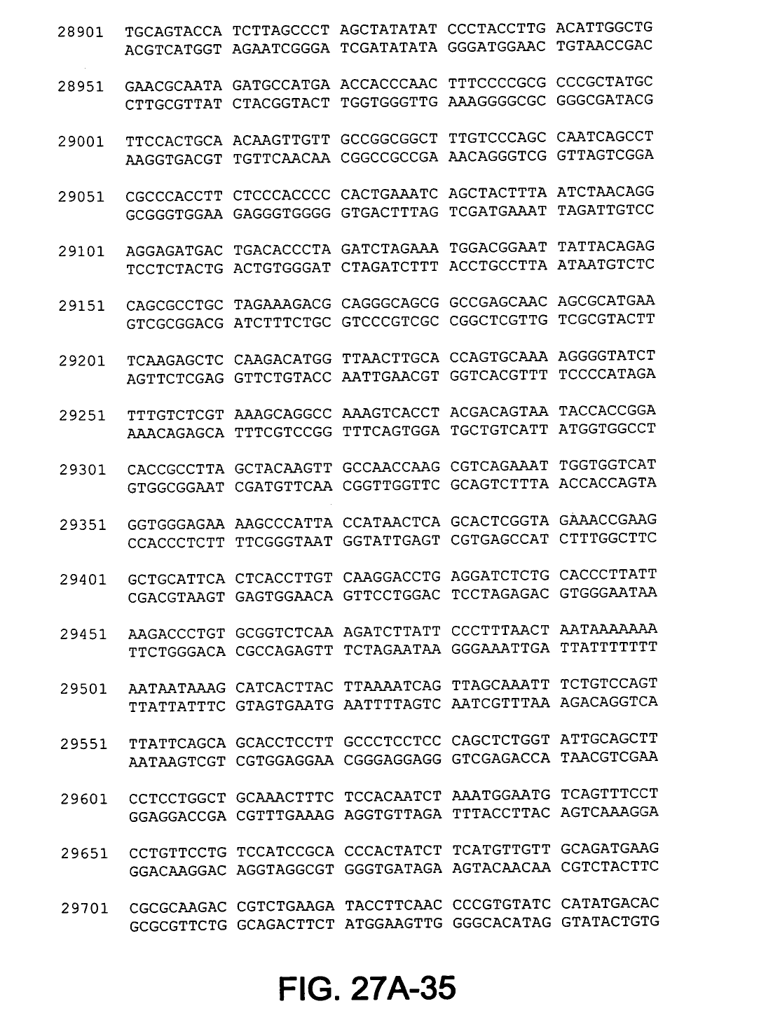

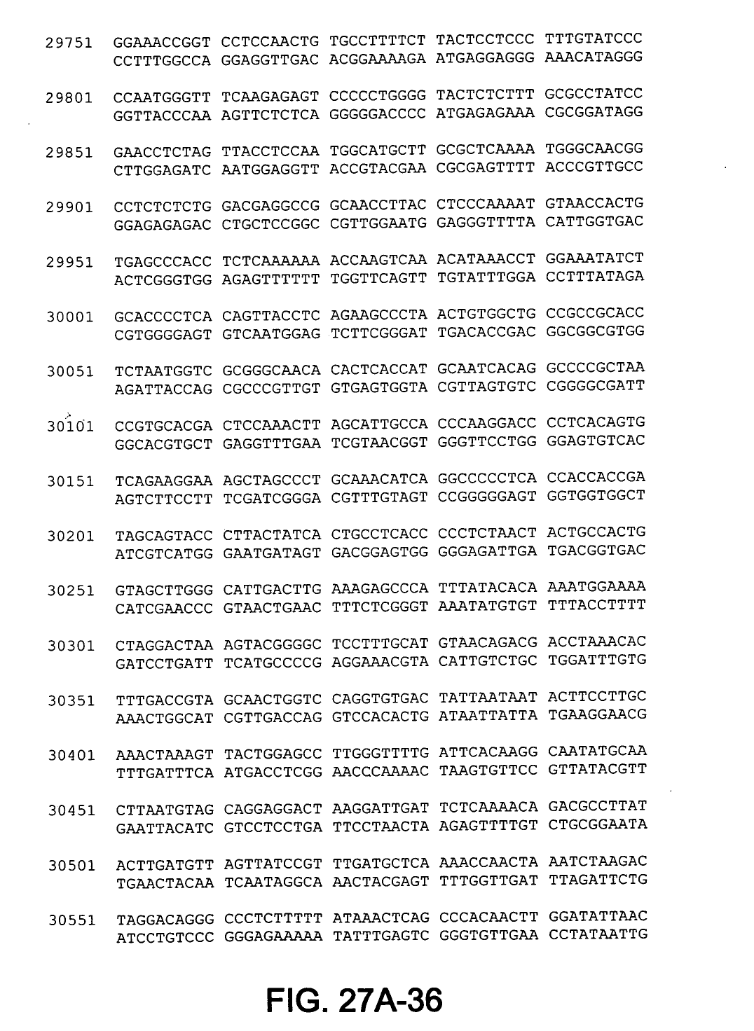

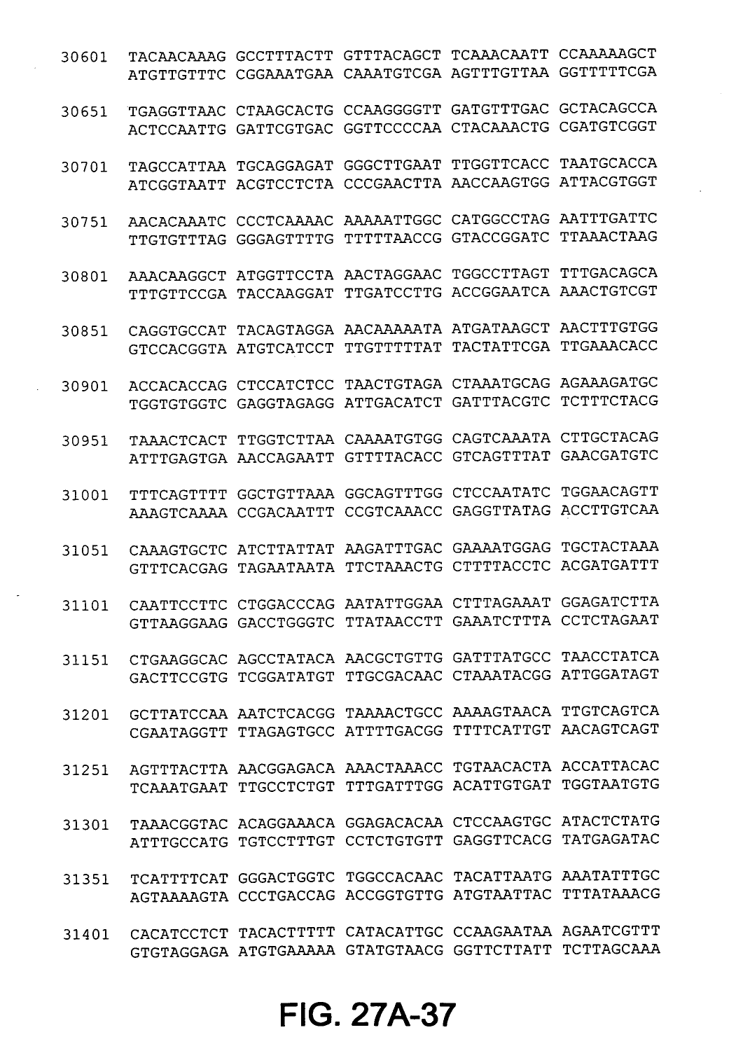

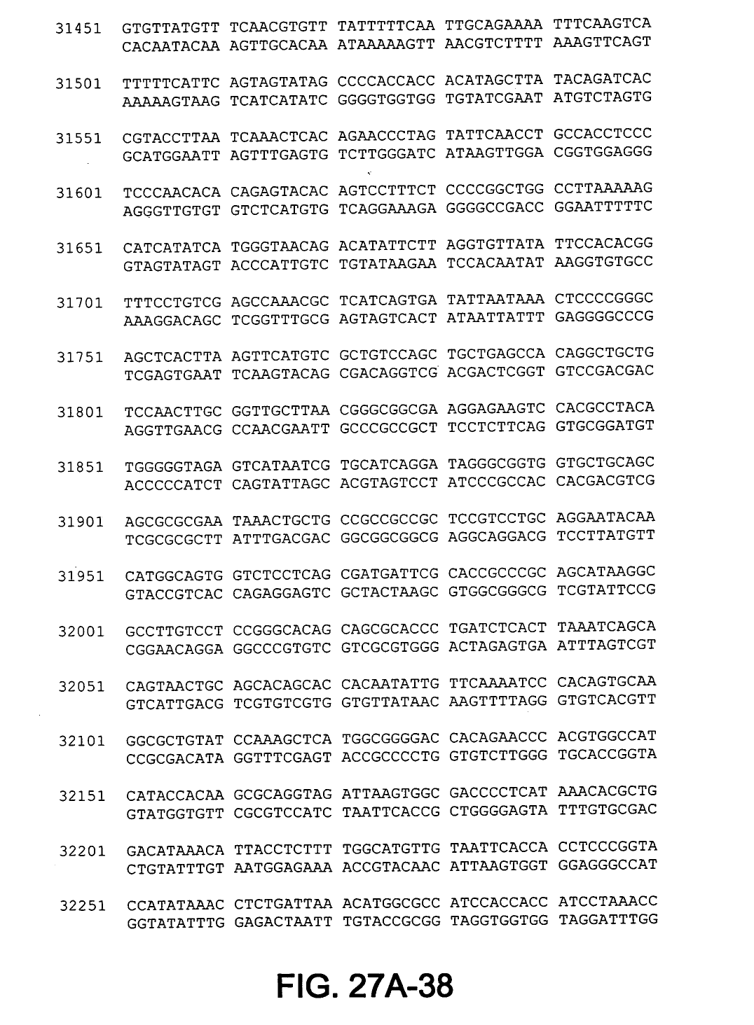

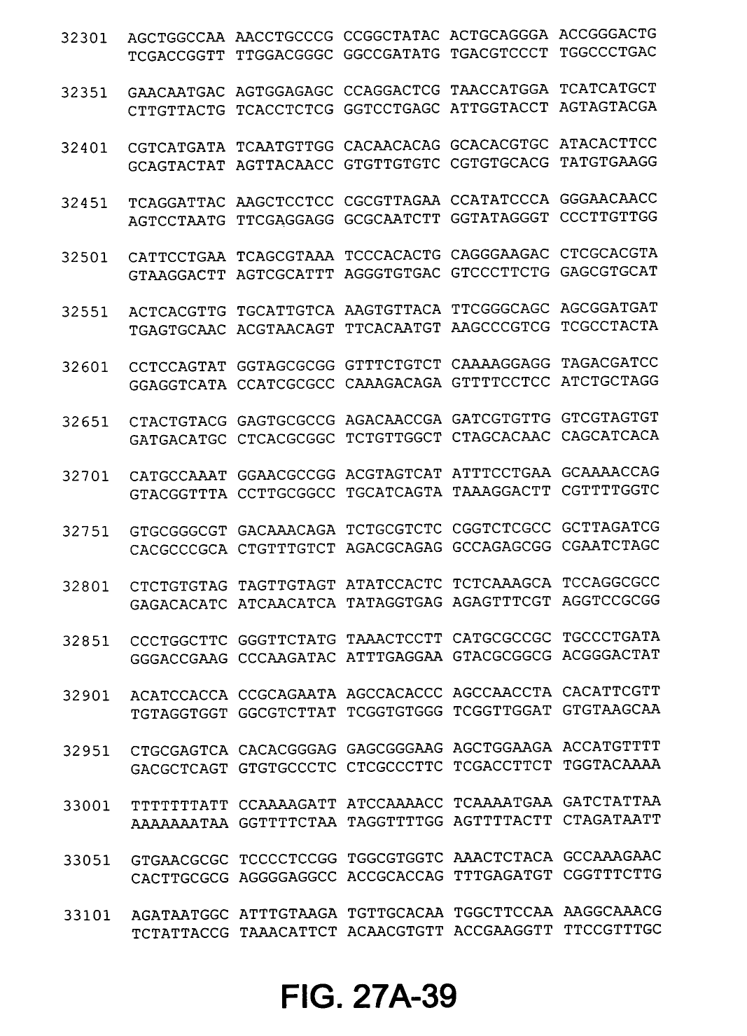

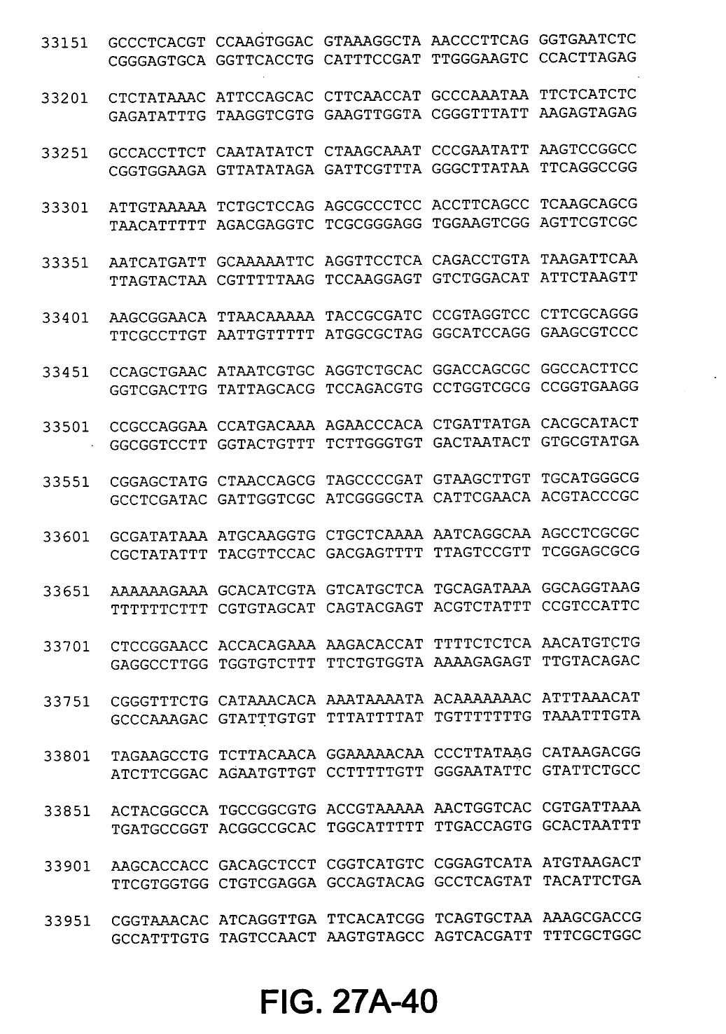

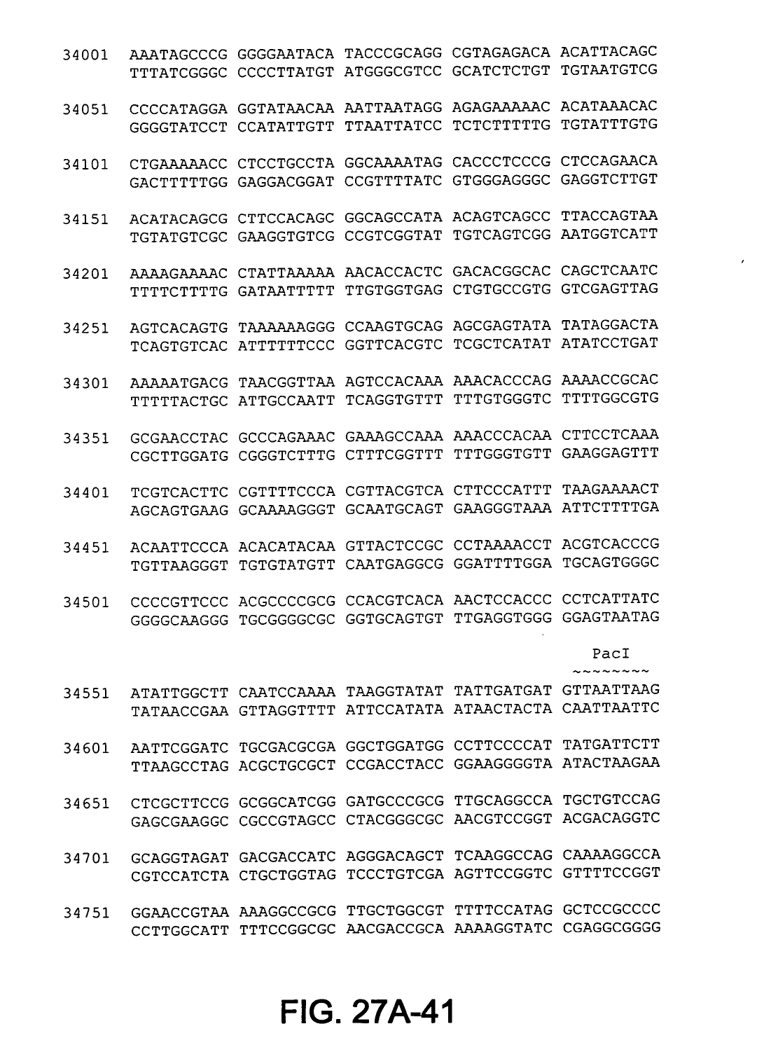

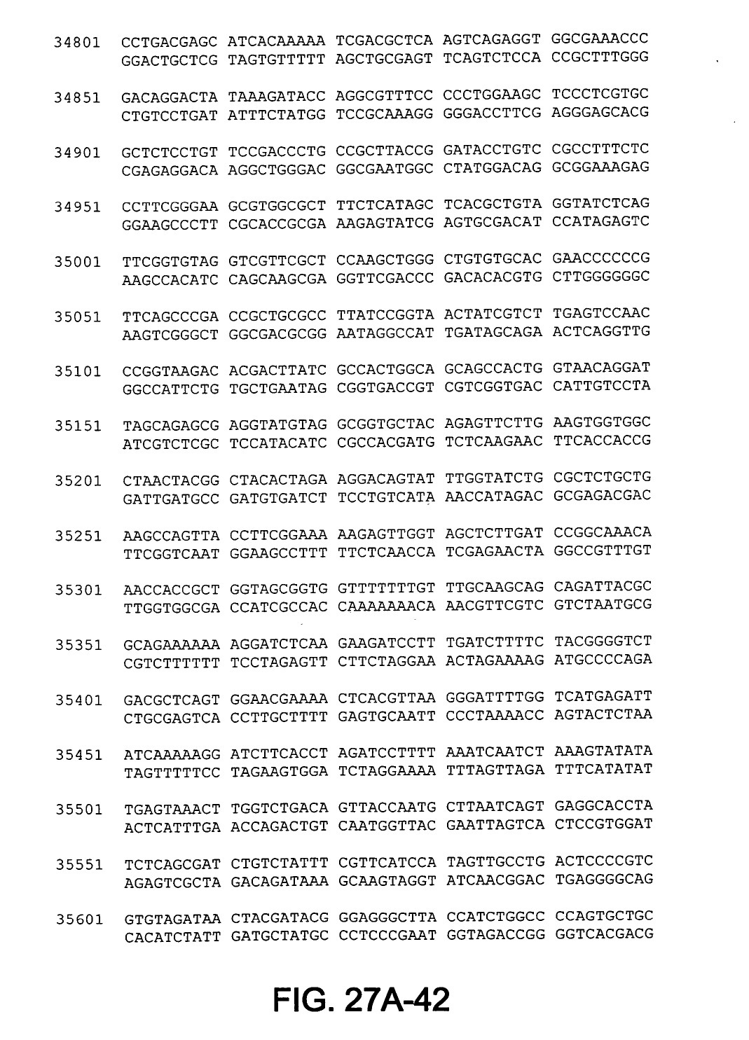

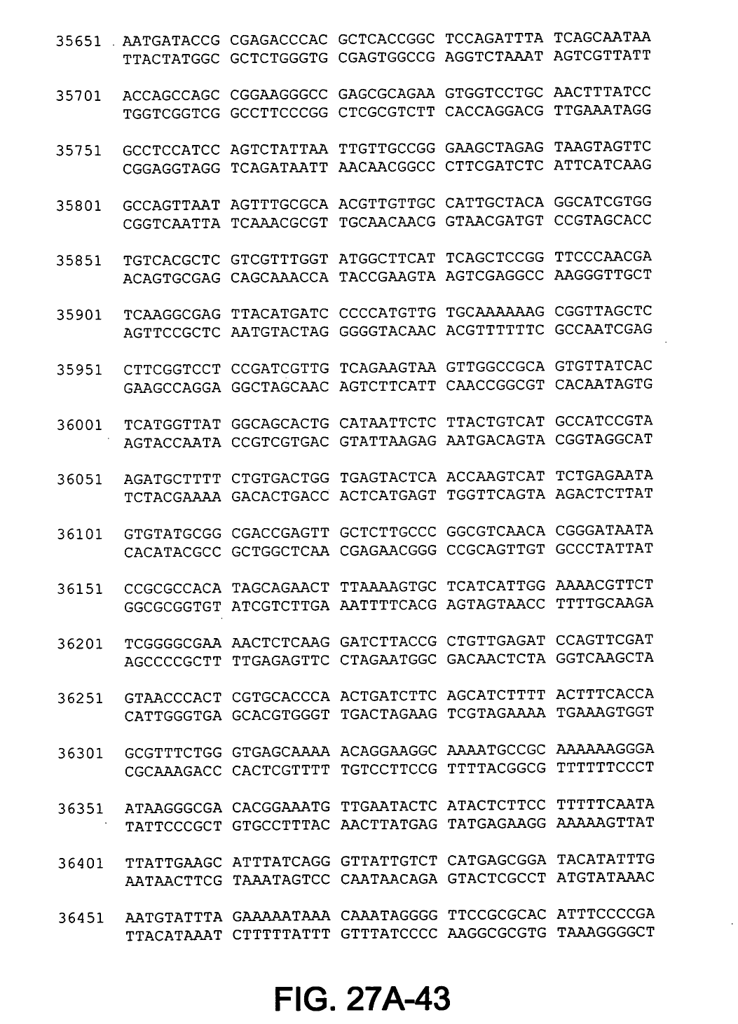

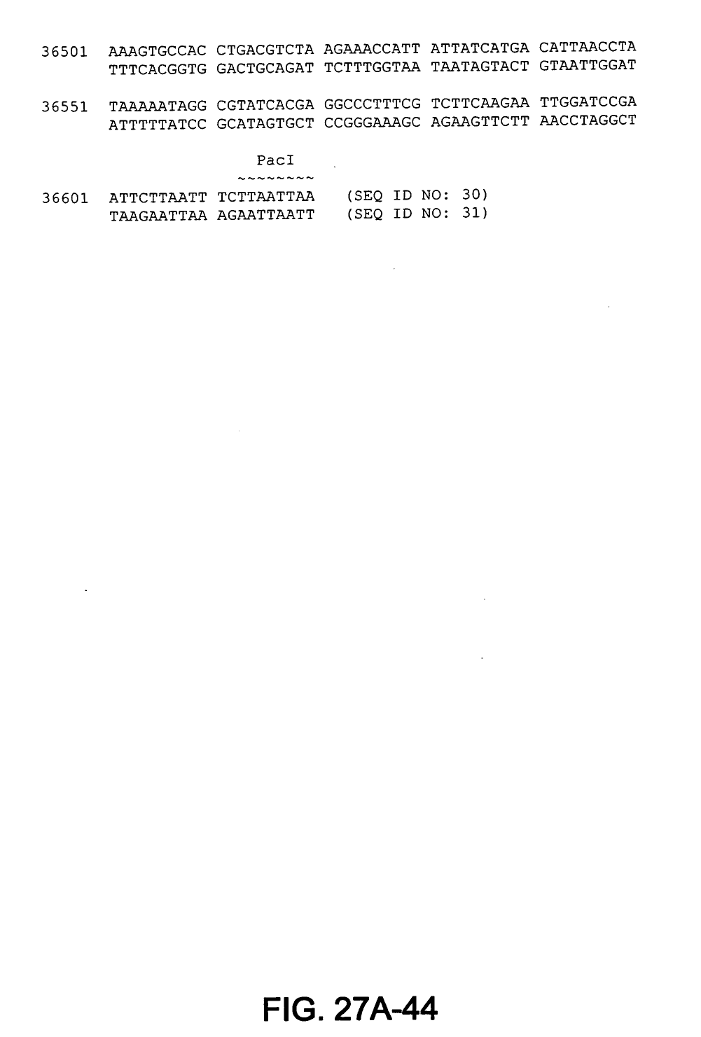

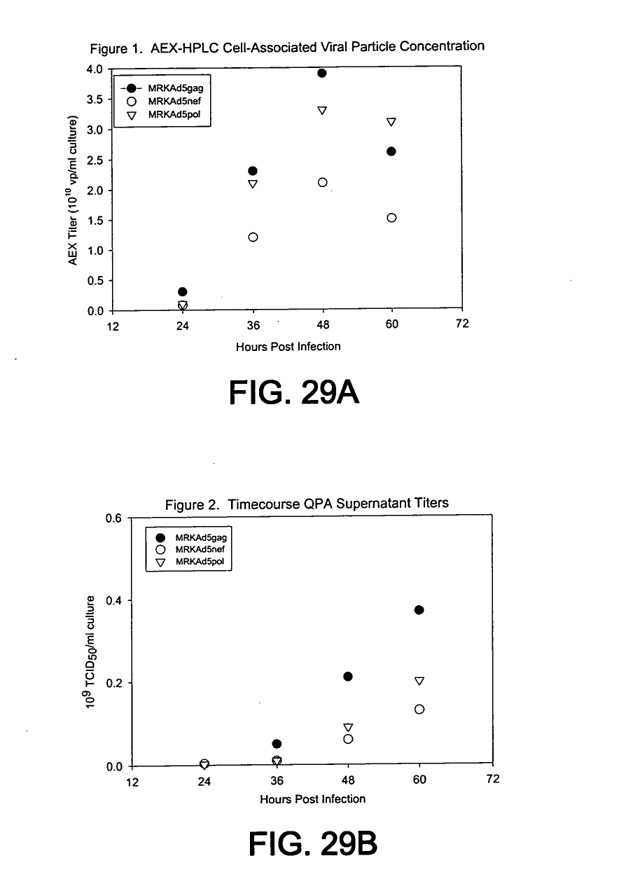

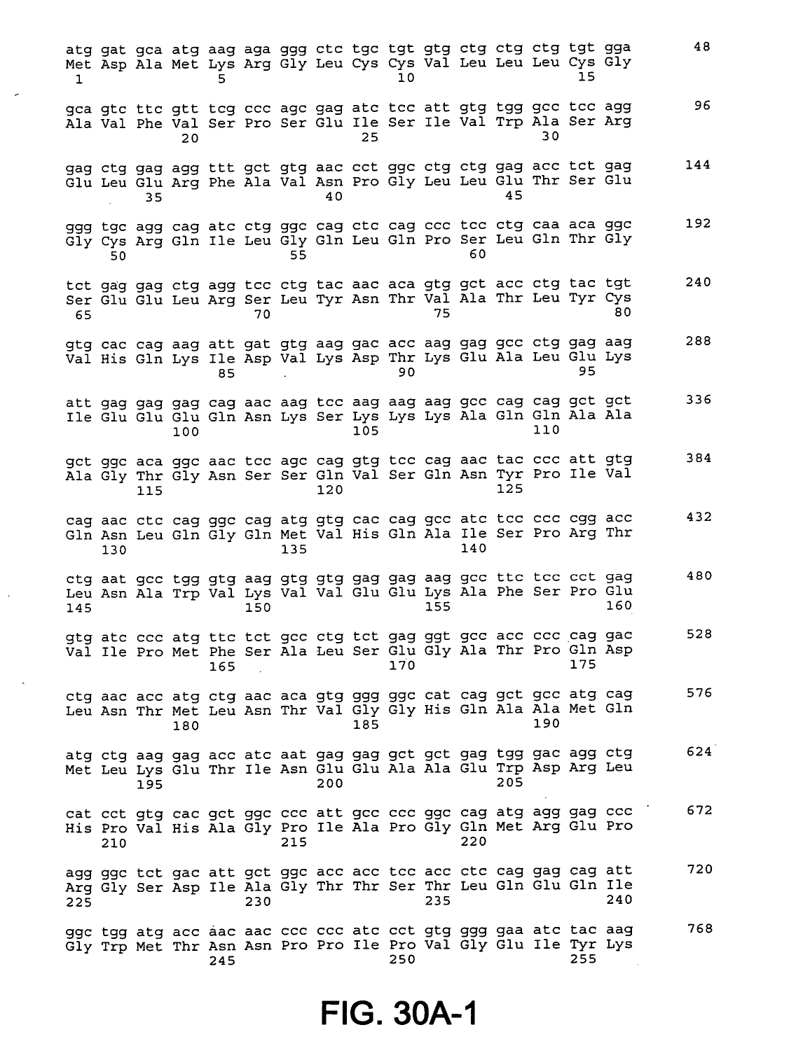

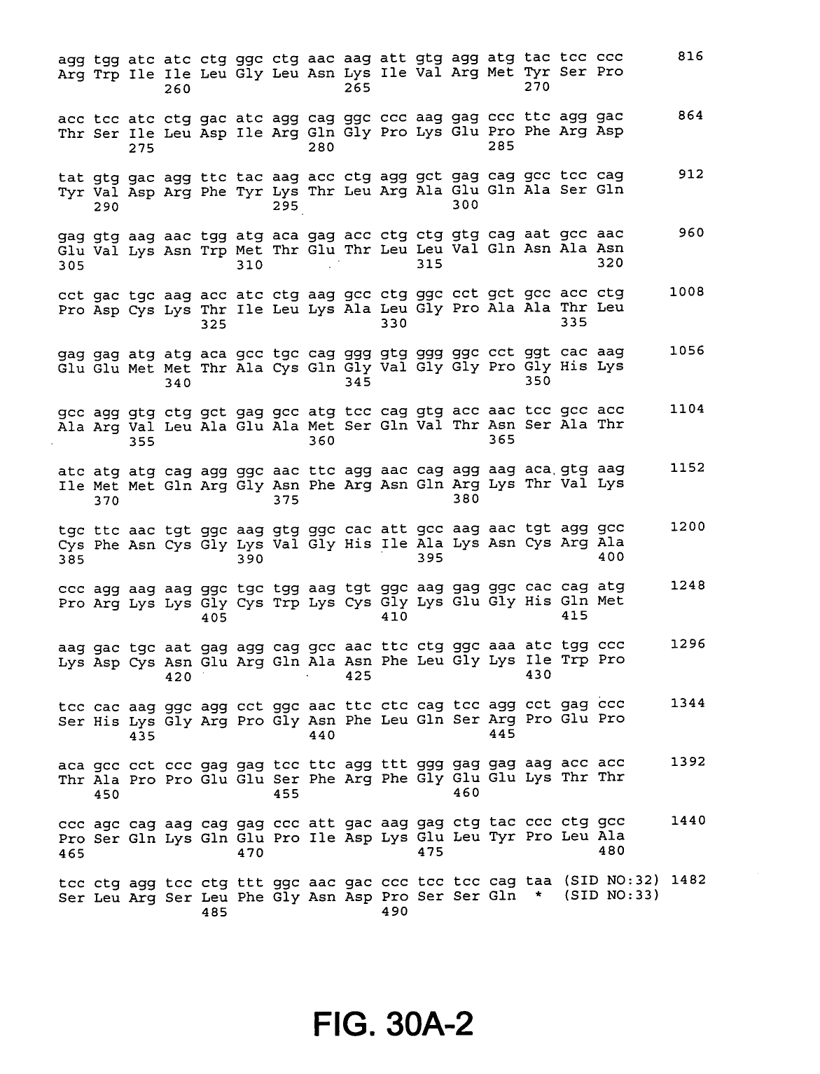

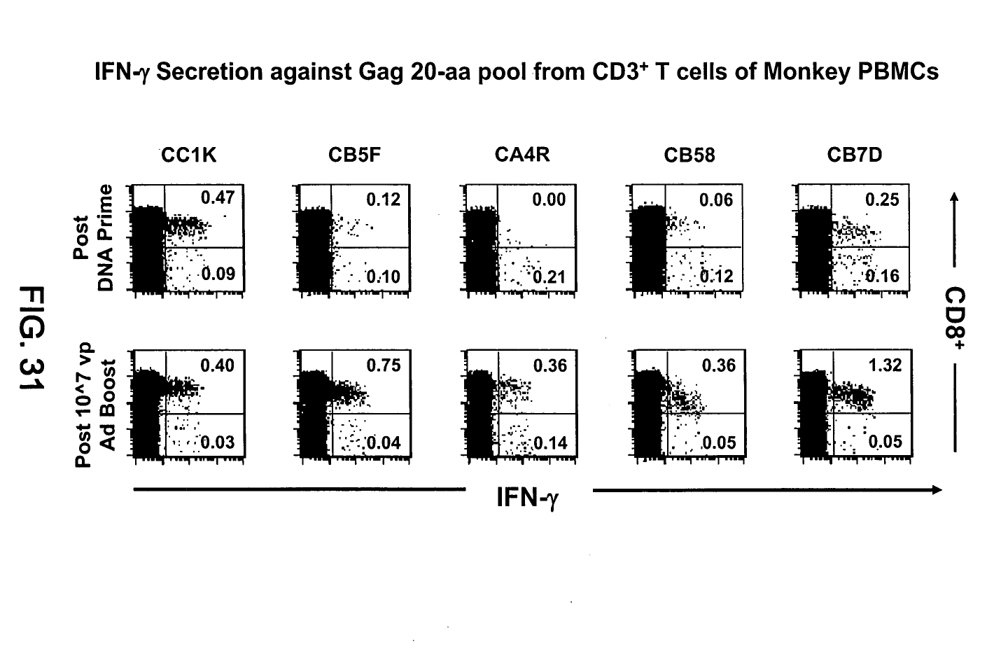

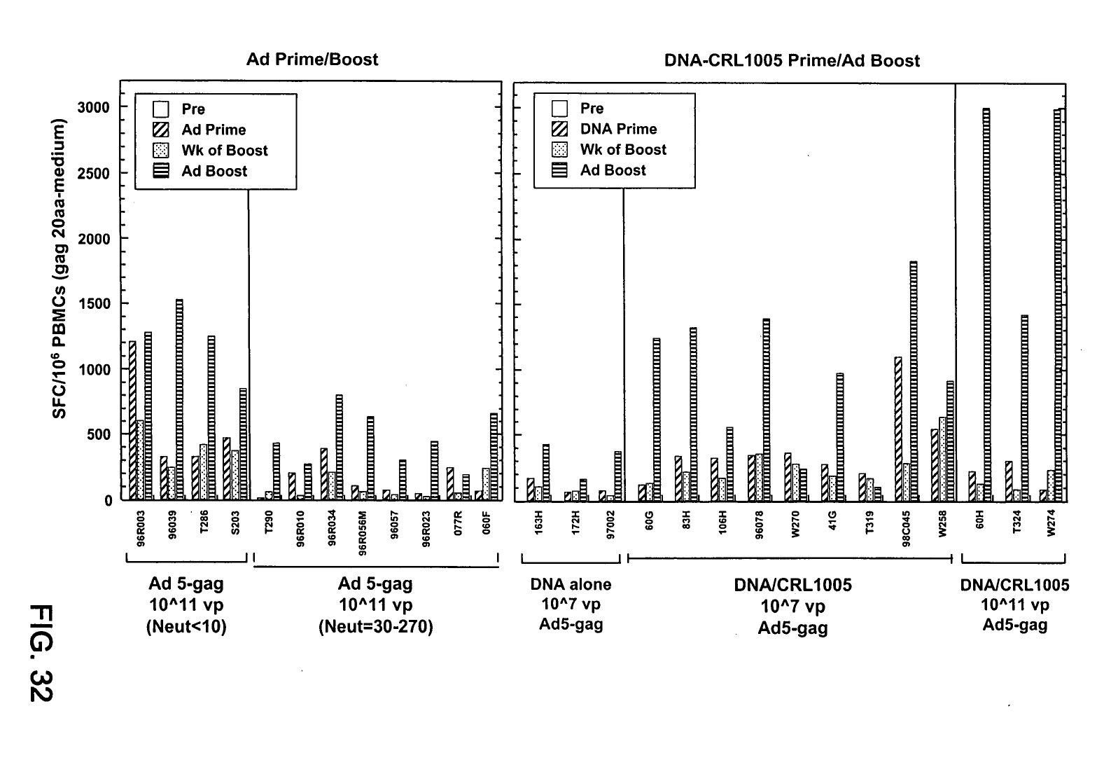

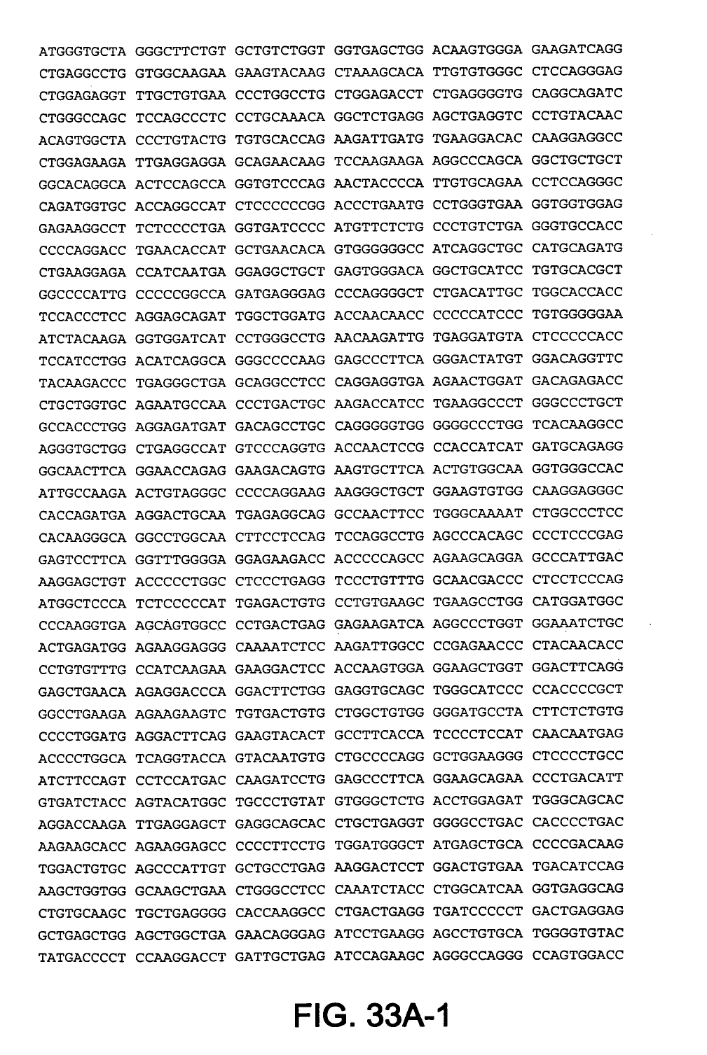

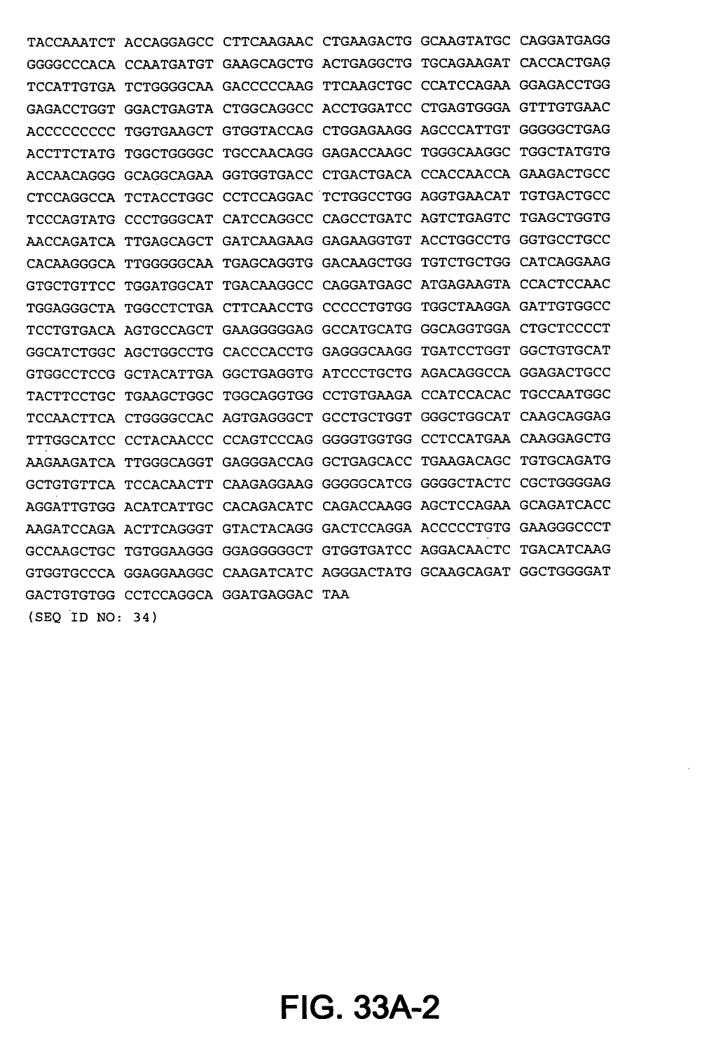

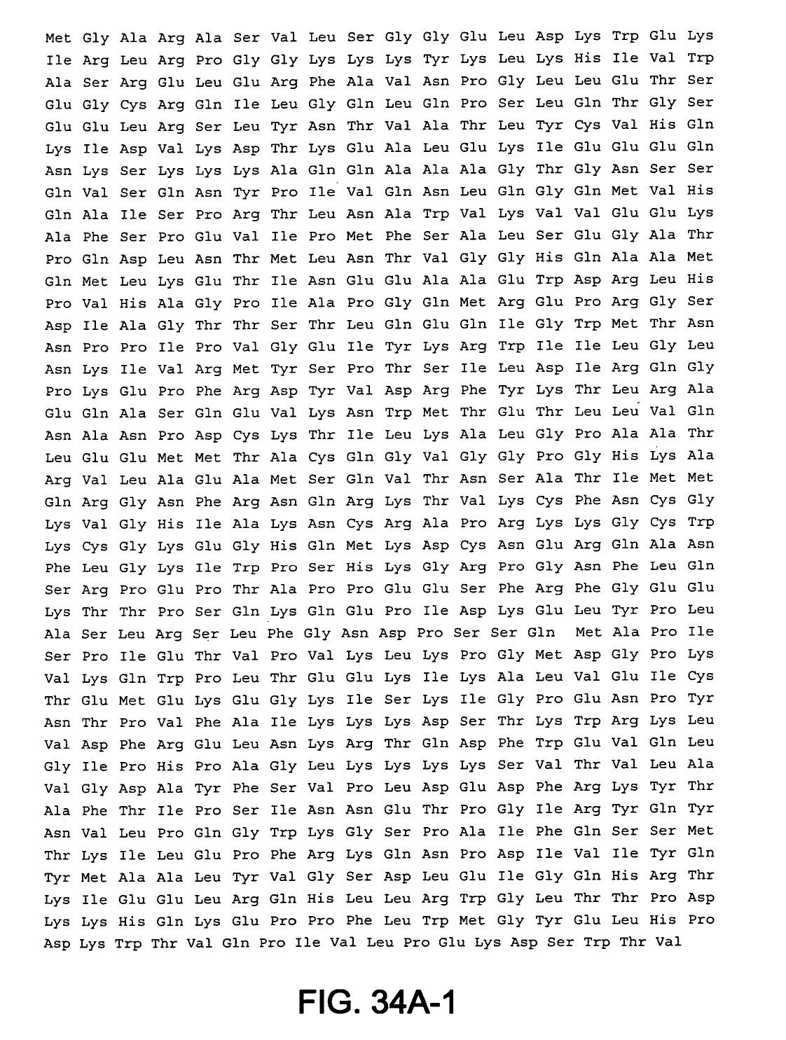

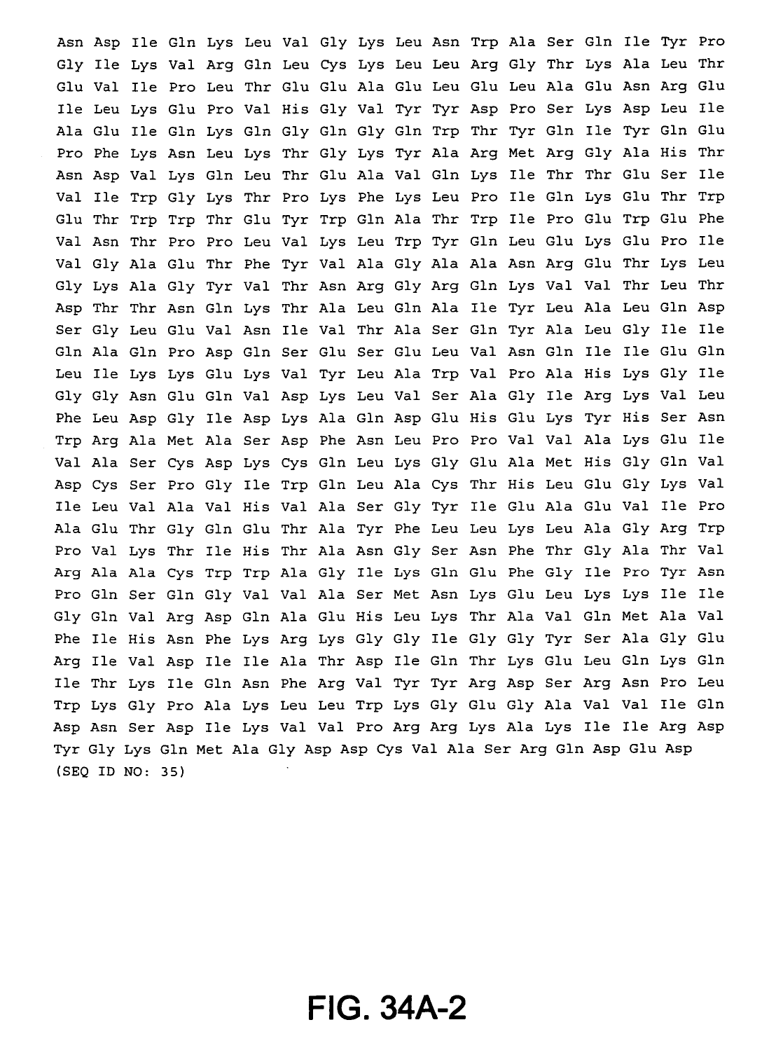

Table 5B shows the amplification rates of the new E3+ vector backbone MRKpAdHVE3. It has a significantly lower rate of amplification compared with the gag transgene containing version. This may be contributed to the larger size MRK Ad5 HIV-1 gag since it contains the transgene. This inclusion of the transgene brings the size of the adenovirus closer to the size of a wild type Ad5 virus. It is well known that adenoviruses amplify best when they are at close to their wild type genomic size. Wild type Ad5 is 35,935 bp. The MRKpAdHVE3 is 32,905 bp in length. The enhanced adenovector MRK Ad5 HIV-I gag is 35,453bp (See FIG. 14 for vector map; see also FIGS. 15A-1 to 15A-45 which show the complete pre-adenoviral vector sequence, which includes an additional 2,021 bp of the vector backbone).

Table 5C shows the amplification rates of the new E3− gag containing virus MRK Ad5 HIV-1 gag E3−. Once again, this virus shows lower growth rate than the enhanced adenoviral vector. This may be attributed to the decreased sized of this virus (due to the E3 gene deletion) compared with wild type Ad5. The MRK Ad5 HIV-1 gag E3− virus is 32,810 bp in length. This can be compared with the wild type Ad5 which is 35,935 bp and MRK Ad5 HIV-1 gag which is 35,453 bp in length.

TABLE 5A
Amplification ratios determined by AEX and QPA for MRKAd5gag over several
continuous passaging in serum free media. Following P5, two replicate samples were taken (rep-
1 and rep-2) and analyzed.
MRKAd5gag rep1

| | Xv (10⁶ cells/ml), | | | | | | | | | AEX |
|---|---|---|---|---|---|---|---|---|---|---|
| | Viability (%) | | Harvest Time | Cell Passage | Titer | Titer | QPA | Ratio | Amplification | Internal |
| | Infection | Harvest | h.p.l. | Number | 10¹⁰ vp/ml culture | 10⁴ vp/cell | 10⁹ TCID₅₀/ml | AEX:QPA | Ratio | Control |
| P4 | 1.49, 81% | 0.58, 50% | 44 | 46 | 8.7 | 5.9 | 1.72 | 50 | 470 | |
| | | | | | | | | | (MOI = 125) | |
| P5 | 1.38, 93% | 0.66, 47% | 48 | 49 | 6.7 | 4.9 | 1.38 | 49 | 170 | |
| P6 | 1.04, 94% | 0.68, 77% | 47 | 48 | 5.8 | 5.6 | 1.42 | 41 | 200 | |
| P7 | 1.50, 84% | 0.96, 61% | 49.5 | 50 | 3.9 | 1.4 | 0.97 | 40 | 50 | |
| P7 | 1.09, 97% | 0.76, 59% | 50 | 52 | 5.2 | 4.7 | 1.70 | 31 | 170 | |
| P8 | 1.03, 94% | 0.86, 64% | 47.5 | 54 | 9.0 | 8.7 | 1.10 | 82 | 310 | |
| P9 | 0.89, 95% | 0.99, 73% | 47.5 | 56 | 4.4 | 4.9 | 1.03 | 43 | 175 | 3.12 |
| | | | | | | | | | | 2.84 |
| P10 | 1.09, 91% | 1.06, 66% | 47.5 | 58 | 3.0 | 2.8 | 1.16 | 26 | 100 | 2.70 |
| | | | | | | | | | | 2.60 |
| P11 | 1.19, 88% | 0.98, 65% | 47 | 60 | 3.6 | 3.0 | 1.15 | 31 | 110 | 2.70 |
| | | | | | | | | | | 2.70 |
| P12 | 0.98, 91% | 0.85, 63% | 47.5 | 47 | 5.4 | 5.5 | 1.20 | 45 | 200 | 2.86 |
| | | | | | | | | | | 2.60 |
| P13 | 1.00, 88% | 0.70, 67% | 49 | 49 | 5.8 | 5.8 | 1.11 | 52 | 210 | 3.18 |
| | | | | | | | | | | 3.18 |
| P14 | 1.94, 92% | 0.88, 67% | 46 | 53 | 8.6 | 4.4 | | | 160 | 3.28 |
| | | | | | | | | | | 3.27 |
| P15 | 0.97, 96% | 0.64, 66% | 47 | 47 | 6.9 | 7.1 | | | 250 | 3.12 |
| | | | | | | | | | | 2.91 |

TABLE 5B
Amplification ratios determined by AEX and QPA for MRKHVE3 over several
continuous passaging in serum free media. MRKHVE3 is the new vector backbone which does
NOT carry a transgene.
MRKHVE3

| | Xv (10⁶ cells/ml), | | | | | | | | | AEX |
|---|---|---|---|---|---|---|---|---|---|---|
| | Viability (%) | | Harvest Time | Cell Passage | Titer | Titer | QPA | Ratio | Amplification | Internal |
| | Infection | Harvest | h.p.l. | Number | 10¹⁰ vp/ml culture | 10⁴ vp/cell | 10⁹ TCID₅₀/ml | AEX:QPA | Ratio | Control |
| P4 | 1.10, 97% | 1.28, 79% | 49 | 54 | 4.1 | 3.8 | 1.70 | 25 | 300 | |
| | | | | | | | | | (MOI = 125) | |
| P5 | 0.92, 89% | 1.18, 77% | 47 | 48 | 4.3 | 4.7 | 1.24 | 35 | 170 | |
| P6 | 1.55, 86% | 1.26, 76% | 49.5 | 50 | 1.2 | 0.8 | 0.56 | 21 | 30 | |
| P6 | 1.09, 97% | 1.11, 81% | 49 | 52 | 4.0 | 3.6 | 1.16 | 34 | 130 | |
| P7 | 1.17, 91% | 1.22, 91% | 47.5 | 54 | 3.7 | 3.2 | 0.50 | 74 | 110 | |
| P8 | 0.98, 88% | 1.41, 83% | 48 | 56 | 2.1 | 2.1 | 0.47 | 45 | 75 | 3.12 |
| | | | | | | | | | | 2.84 |
| P9 | 1.20, 89% | 1.26, 81% | 47.5 | 58 | 0.8 | 0.7 | 0.29 | 28 | 25 | 2.70 |
| | | | | | | | | | | 2.60 |
| P10 | 0.99, 82% | 1.55, 86% | 47 | 60 | 2.3 | 2.3 | 0.43 | 53 | 80 | 2.70 |
| | | | | | | | | | | 2.70 |
| P11 | 1.07, 96% | 1.25, 83% | 48 | 47 | 2.7 | 2.5 | 0.41 | 66 | 90 | 2.86 |
| | | | | | | | | | | 2.60 |
| P12 | 0.80, 91% | 1.14, 80% | 49.5 | 49 | 5.9 | 7.4 | 0.48 | 123 | 260 | 3.18 |
| | | | | | | | | | | 3.18 |
| P13 | 1.96, 95% | 1.14, 85% | 45.5 | 53 | 5.8 | 3.0 | | | 110 | 3.28 |
| | | | | | | | | | | 3.27 |
| P14 | 0.97, 96% | 1.03, 98% | 48.5 | 47 | 9.4 | 9.7 | | | 350 | 3.12 |
| | | | | | | | | | | 2.91 |
| P15 | 0.87, 99% | 0.97, 59% | 49.5 | 49 | 5.3 | 6.1 | | | 218 | 2.78 |
| | | | | | | | | | | 2.52 |

TABLE 5C
Amplification ratios determined by AEX and QPA for MRKAd5gag(E3−) over several continuous passaging
in serum free media. This construct is identical to the MRKAd5gag construct except that this version is DELETED of
the E3 gene.
MRKAd5gag(E3−)

| | Xv (10⁶ cells/ml), | | | | | | | | | AEX |
|---|---|---|---|---|---|---|---|---|---|---|
| | Viability (%) | | Harvest Time | Cell Passage | Titer | Titer | QPA | Ratio | Amplification | Internal |
| | Infection | Harvest | h.p.l. | Number | 10¹⁰ vp/ml culture | 10⁴ vp/cell | 10⁹ TCID₅₀/ml | AEX:QPA | Ratio | Control |
| P4 | 1.62, 77% | 1.12, 62% | 47.5 | 46 | 2.0 | 1.2 | 0.92 | 20 | 100 | |
| | | | | | | | | | (MOI = 125) | |
| P5 | 1.16, 92% | 0.62, 43% | 49 | 49 | 3.3 | 2.9 | 0.99 | 34 | 100 | |
| P6 | 1.71, 86% | 0.20, 10% | 49 | 50 | 4.7 | 2.7 | 1.70 | 28 | 100 | |
| P6 | 1.09, 97% | 0.63, 54% | 49.5 | 52 | 5.4 | 5.0 | 1.76 | 31 | 180 | |
| P7 | 1.17, 91% | 0.98, 72% | 47.50 | 54 | 7.1 | 6.1 | 0.67 | 106 | 220 | |
| P8 | 0.98, 88% | 0.77, 48% | 48 | 56 | 3.1 | 3.2 | 0.66 | 47 | 115 | 3.12 |
| | | | | | | | | | | 2.84 |
| P9 | 1.20, 89% | 1.03, 72% | 48 | 58 | 1.8 | 1.5 | 0.57 | 32 | 55 | 2.70 |
| | | | | | | | | | | 2.60 |
| P10 | 0.99, 82% | 0.80, 62% | 46.5 | 60 | 3.2 | 3.2 | 0.68 | 47 | 115 | 2.70 |
| | | | | | | | | | | 2.70 |
| P11 | 1.07, 96% | 0.98, 70% | 48.5 | 47 | 5.9 | 5.5 | 0.68 | 87 | 200 | 2.86 |
| | | | | | | | | | | 2.60 |
| P12 | 0.80, 91% | 0.67, 59% | 50 | 49 | 5.1 | 6.4 | 0.72 | 71 | 230 | 3.18 |
| | | | | | | | | | | 3.18 |
| P13 | 1.96, 95% | 0.91, 59% | 45.5 | 53 | 7.4 | 3.8 | | | 135 | 3.28 |
| | | | | | | | | | | 3.27 |
| P14 | 0.97, 96% | 0.81, 74% | 48 | 47 | 6.8 | 7.0 | | | 250 | 3.12 |
| | | | | | | | | | | 2.91 |
| P15 | 0.87, 99% | 0.84, 56% | 49 | 49 | 4.8 | 5.5 | | | 196 | 2.78 |
| | | | | | | | | | | 2.52 |

### EXAMPLE 14

### Gag Expression Analysis of the Novel Constructs

In vitro gag analysis of the MRK Ad5 HIV-1 gag and the original HIV-gag vectors (research and clinical lot) show comparable gag expression. The clinical lot shows only a slightly reduced gag expression level. The most noticeable difference is with the mCMV vector. This vector shows roughly 3 fold lower expression levels compared with the other vectors tested (which all contain hCMV promoters). The mCMV-FLgag with bGHpA assay was performed three times using different propagation and purification lots and it consistently exhibited weaker gag expression.

### EXAMPLE 15

### Evaluation of MRK Ad5 HIV-1 gag and Other gag-Containing Adenovectors in Balb/c Mice

Cohorts of 10 balb/c mice were vaccinated intramuscularly with escalating doses of MRK Ad5 HIV-1 gag, and the research and clinical lots of original Ad5HIV-1gag. Serum samples were collected 3 weeks post dose 1 and analyzed by anti-p24 sandwich ELISA.

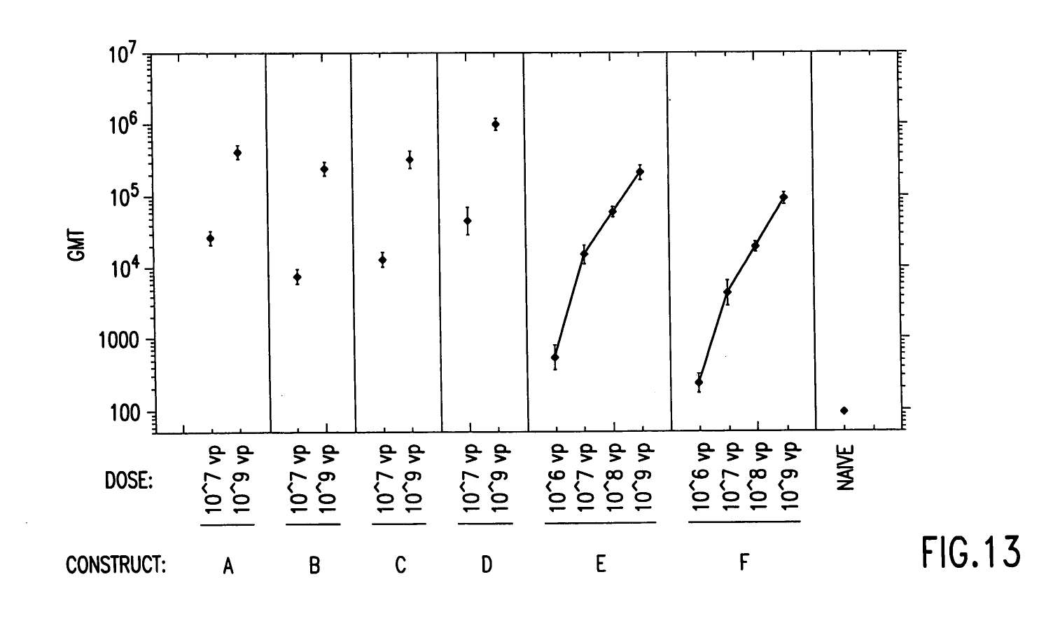

Anti-p24 titers in mice that received MRK Ad5 HIV-1 gag (10⁷ and 10⁹ vp(viral particle) doses) were comparable (FIG. 13) to those of the research lot of Ad5HIV-1 gag, for which much of the early rhesus data were generated on. These titers were also comparable when E3 is deleted (MRKAd5hCMVgagbGHpA(E3−)) or SPA is substituted for bGHpA terrninator (MRKAd5 hCMV-gag-SPA (E3+)) or murine CMV promoter is used in place of hCMV (MRKAd5 mCMV-gag-bGHpA (E3+)) in the MRKAd5 backbone.

The results shown in Table 7 indicate that the three other vectors (in addition to the preferred vector, MRK Ad5 HIV-1 gag, are also capable of inducing strong anti-gag antibody responses in mice. Interestingly enough, while the mCMV-FLgag construct containing bGHpA and E3+ in an E1 parallel orientation showed lowest gag expression in the COS cell in vitro infection (Table 6) in comparison with the other vectors tested, it generated the greatest anti-gag antibody response this in vivo Balb/c study. Table 7 also shows a dose response in anti-gag antibody production in both the research and the clinical lot. As expected, the clinical lot shows reduced anti-gag antibody induction at each dosage level compared to the same dosage used for the research lot.

TABLE 6
In vitro analysis for gag expression in COS cells by Elisa assay.

| Viral Vectors^{a} | μg gag/4.8 × 10e5 COS/10e8 parts/48 hr |
|---|---|
| MRKAd5gag^{b} | 1.40 |
| Clinical lot Ad5gag^{c} | 1.28 |
| Research lot Ad5gag^{d} | 1.32 |
| MCMVFL-gagbGHpA^{e} | 0.42 |

^{a}A₂₆₀ₙₘ absorbance readings taken for viral particle determinations.
^{b}MRKAd5gag was produced in serum free conditions and purified at P5.
^{c}Clinical lot# Ad5gagFN0001
^{d}Research Ad5FLgag lot# 6399
^{e}mCMVFL-gagbGHpA was produced in serum free conditions and purified at P5.

TABLE 7
mHIV020 Anti-p24 Ab Titers in Balb/c mice (n = 10) vaccinated with various Adgag
constructs and lots (3 week post dose 1).

| Group | | Dose | | | |
|---|---|---|---|---|---|
| ID | Vaccine | (vp) | GMT | SE upper | SE lower |
| 1 | ^{a}MRKAd5gag | 10\hat{ }7 | 25600 | 5877 | 4780 |
| 2 | ″ | 10\hat{ }9 | 409600 | 94028 | 76473 |
| 3 | hCMV FL-gag bGHpA [E3−] → | 10\hat{ }7 | 7352 | 2077 | 1620 |
| 4 | ″ | 10\hat{ }9 | 235253 | 59767 | 47659 |
| 5 | hCMV FL-gag SPA [E3+] → | 10\hat{ }7 | 12800 | 9905 | 236 |
| 6 | ″ | 10\hat{ }9 | 310419 | 99181 | 75165 |
| 7 | ^{b}mCMV FL-gag bGHpA [E3+] → | 10\hat{ }7 | 44572 | 23504 | 15389 |
| 8 | ″ | 10\hat{ }9 | 941014 | 239068 | 190636 |
| 9 | ^{c}hCMV FL-gag bGHpA [E3−] [symbol] | 10\hat{ }7 | 3676 | 934 | 745 |
| 10 | ″ | 10\hat{ }9 | 117627 | 17491 | 15227 |
| 11 | research lot hCMV intronA FL-gag bGHpA [E3−] <- | 10\hat{ }6 | 528 | 262 | 175 |
| 12 | ″ | 10\hat{ }7 | 14703 | 5274 | 3882 |
| 13 | ″ | 10\hat{ }8 | 58813 | 14942 | 11915 |
| 14 | ″ | 10\hat{ }9 | 204800 | 53232 | 42250 |
| 15 | clinical lot hCMVintronA FL-gag bGHpA [E3−] <- | 10\hat{ }6 | 230 | 82 | 61 |
| 16 | ″ | 10\hat{ }7 | 4222 | 3405 | 1138 |
| 17 | ″ | 10\hat{ }8 | 19401 | 3939 | 3274 |
| 18 | ″ | 10\hat{ }9 | 89144 | 25187 | 19639 |
| 19 | Naïve | none | 93 | 7 | 6 |

*2 × 50 μL i.m. (quad) injections/animal
P.I.s: Youil, Chen, Casimiro
Vaccination: T. Toner, Q. Su
Assay: M. Chen
^{a}The structure of MRKAd5gag is: hCMVFL-gagbGHpA [E3+] → The same lot of MRKAd5gag used in this rodent study was used in the Rhesus monkey study (Tables 7 and 8).
^{b}The same lot of mCMVFL-gagbGHpA[E3+] used in the in vitro study (Table 6) ws used here.
^{c}This construct was designed by Volker Sandig. It contains a shorter version of the hCMV promoter than that used in the MRK constructs. The adenovector backbone is identical to the original backbone used in the original Adgag vector. Expression at 10e7 dose from this vector is 7 fold lower then the same dose of the MRKAd5gag and 4 fold lower than the research lot.

### EXAMPLE 16

### Comparison of Humoral and Cellular Responses Towards the Original Ad-gag Construct with the New MRK Ad5 HIV-1 gag in Rhesus Monkeys

Cohorts of 3 rhesus monkeys were vaccinated intramuscularly with MRK Ad5 HIV-1 gag or the clinical Ad5gag bulk at two doses, 10″ vp and 10⁹ vp. Immunizations were conducted at week 0, 4, and 25. Serum and PBMC samples were collected at selected time points. The serum sample were assayed for anti-p24 Ab titers (using competitive based assay) and the PBMCs for antigen-specific IN-gamma secretion following overnight stimulation with gag 20-mer peptide pool (via ELISpot assay).

The results shown in Table 8 indicate comparable responses with respect to the generation of anti-gag antibodies. The frequencies of gag-specific T cells in peripheral blood as summarized in Table 9 demonstrate a strong cellular immune response generated after a single dose with the new construct MRK

Ad5 HIV-1 gag. The responses are also boostable with second dose of the same vector. The vector is also able to induce CD8+ T cell responses (as evident by remaining spot counts after CD4+ depletion of PBMCs) which are responsible for cytotoxic activity.

TABLE 8
Anti-p24 antibody titers (in mMU/mL) in rhesus macaques immunized with gag-expressing
adenovectors (Protocol HIV203).

| Vaccine | Pre | Wk 4 | Wk 8 | Wk 12 | Wk 16 | Wk 20 | Wk 25 | Wk 28 |
|---|---|---|---|---|---|---|---|---|
| MRKAd5gag^{a}, 10\hat{ }11 vp | | | | | | | | |
| 97N010 | <10 | 118 | 5528 | 11523 | 7062 | 21997 | ND | 51593 |
| 97N116 | <10 | 62 | 772 | 1447 | 1562 | 2174 | ND | 20029 |
| 98X007 | <10 | 66 | 3353 | 6156 | 6845 | 3719 | ND | 24031 |
| MRKAd5gag, 10\hat{ }9 vp | | | | | | | | |
| 97N120 | <10 | 51 | 204 | 318 | 366 | 482 | ND | 6550 |
| 97N144 | <10 | 18 | 118 | 274 | 706 | 888 | ND | 7136 |
| 98X008 | <10 | 15 | 444 | 386 | 996 | 1072 | ND | 12851 |
| Ad5gag^{b}, Clinical Lot, 10\hat{ }11 vp | | | | | | | | |
| 97X001 | <10 | 87 | 2579 | 4718 | 7174 | 7250 | ND | 69226 |
| 97N146 | <10 | 72 | 3604 | 7380 | 7526 | 18906 | ND | 60283 |
| 98X009 | <10 | 78 | 4183 | 3946 | 3124 | 6956 | ND | 26226 |
| Ad5gag, Clinical Lot, 10\hat{ }9 vp | | | | | | | | |
| 97N020 | <10 | <10 | 143 | 371 | 390 | 1821 | ND | 17177 |
| 97X003 | <10 | <10 | 39 | 93 | 156 | 596 | ND | 2053 |
| 98X012 | <10 | 81 | 342 | 717 | 956 | 1558 | ND | 11861 |

^{a}MRKAd5gag (hCMV, bGHpA, E3+)
^{b}original Ad5gag vector (hCMV/Intron A, bGHpA, E3−), lot#FN0001
ND, not determined

TABLE 9
Number of gag-specific T cells per million peripheral blood mononuclear cells (PBMCs) in
rhesus monkeys immunized with gag-expressing adenovectors. Also included are those frequencies in
PBMCs depleted of CD4⁺ T cells.

| | | | T = 4 Wk | | T = 6 Wk | | T = 11 Wk | | T = 16 Wk | | T = 25 Wk | | T = 28 Wk | |
|---|---|---|---|---|---|---|---|---|---|---|---|---|---|---|
| | Vaccination | | | Gag | | Gag | | Gag | | Gag | | Gag | | Gag |
| Grp # | T = 0, 4, 25 wks | Monkey ID | Media^{a} | H^{b} | Media | H | Media | H | Media | H | Media | H | Media | H |
| 1 | MRKAd5gag | 97N010 | 6 | 89 | 0 | 395 | 0 | 1058 | 0 | 1174 | 3 | 775 | 4 | 1074 |
| | 10\hat{ }11 vp | 97N010(CD4−) | 4 | 38 | | | 3 | 993 | | | 0 | 76 | 0 | 594 |
| | | 97N116 | 1 | 396 | 1 | 609 | 0 | 534 | 4 | 395 | 1 | 261 | 0 | 408 |
| | | 97N116(CD4−) | 11 | 676 | | | 0 | 593 | | | 0 | 184 | 0 | 666 |
| | | 98X007 | 10 | 579 | 0 | 1304 | 3 | 2193 | 1 | 2118 | 3 | 1588 | 0 | 2113 |
| | | 98X007(CD4−) | 20 | 965 | | | 0 | 2675 | | | 0 | 1656 | 0 | 1278 |
| 2 | MRKAd5gag | 97N120 | 5 | 275 | 1 | 249 | 4 | 141 | 4 | 119 | 9 | 206 | 4 | 219 |
| | 10\hat{ }9 vp | 97N120(CD4−) | 11 | 170 | | | 0 | 85 | | | 0 | 75 | 1 | 219 |
| | | 97N144 | 3 | 236 | 6 | 438 | 1 | 318 | 3 | 256 | 1 | 98 | 5 | 373 |
| | | 97N144(CD4−) | 6 | 148 | | | 0 | 285 | | | ND | ND | 0 | 625 |
| | | 98X008 | 4 | 368 | 1 | 1090 | 3 | 891 | 4 | 673 | 3 | 473 | 5 | 735 |
| | | 98X008(CD4−) | 14 | 696 | | | 0 | 1175 | | | 0 | 391 | 4 | 848 |
| 3 | Ad5gag clinical lot | 97X001 | 0 | 261 | 1 | 485 | 0 | 817 | 0 | 1220b | 1 | 894 | 0 | 1858 |
| | 10\hat{ }11 vp | 97X001(CD4−) | 10 | 283 | | | 3 | 996 | | | 0 | 1010 | 0 | 1123 |
| | | 97N146 | 3 | 150 | 1 | 465 | 0 | 339 | 1 | 1272 | 3 | 1238 | 3 | 1785 |
| | | 97N146(CD4−) | 6 | 133 | | | 0 | 370 | | | 0 | 654 | 0 | 971 |
| | | 98X009 | 0 | 93 | 3 | 339 | 3 | 559 | 0 | 896 | 1 | 384 | 0 | 1748 |
| | | 98X009(CD4−) | 0 | 73 | | | 0 | 333 | | | 0 | 225 | 0 | 644 |
| 4 | Ad5gag clinical lot | 97N020 | 3 | 30 | 1 | 101 | 0 | 66 | 0 | 36 | 0 | 26 | 0 | 41 |
| | 10\hat{ }9 vp | 97N020(CD4−) | 10 | 29 | | | 0 | 15 | | | 0 | 1 | 0 | 16 |
| | | 97X003 | 4 | 68 | 5 | 134 | 0 | 18 | 1 | 38 | 4 | 38 | 6 | 81 |
| | | 97X003(CD4−) | 9 | 40 | | | 0 | 6 | | | 0 | 4 | 0 | 19 |
| | | 98X012 | 5 | 95 | 3 | 54 | 1 | 34 | 0 | 18 | 0 | 20 | 1 | 121 |
| | | 98X012(CD4−) | 11 | 70 | | | 0 | 11 | | | 0 | 8 | 0 | 41 |
| 5 | Naïve | 96R041 | 6 | 8 | 1 | 1 | 0 | 0 | 0 | 0 | 0 | 0 | 1 | 0 |
| | | 053F | 14 | 18 | 5 | 16 | 20 | 14 | 19 | 15 | 10 | 15 | 24 | 9 |

Based on either 4 × 10\hat{ }5 or 2 × 10\hat{ }5 cells per well (depending on spot density)
ND, not determined
^{a}mock or no peptide control
^{b}Pool of 20-aa peptides overlapping by 10 aa and encompassing the gag sequence

The adenovectors described herein and, particularly, MRK Ad5 HIV-1 gag, represent very promising HIV-gag adenovectors with respect to their enhanced growth characteristics in both serum and, more importantly, in serum-free media conditions. In comparison with the current HI-1 gag adenovector construct, MRK Ad5 HIV-1 gag shows a 5-10 fold increased amplification rate. We have shown that it is genetically stable at passage 21. This construct is able to generate significant cellular immune responses in vivo even at a relatively low dose of 10ˆ9vp. The potency of the MRKAd5gag construct is comparable to, if not better than the original HIV-1gag vector as shown in this rhesus monkey study.

EXAMPLE 17

### Codon Optimized HIV-1 Pol and Codon Optimized HIV-1 Pol Modifications

The open reading frames for the various synthetic pol genes disclosed herein comprise coding sequences for the reverse transcriptase (or RT which consists of a polymerase and RNase H activity) and integrase (IN). The protein sequence is based on that of Hxb2r, a clonal isolate of IIIB; this sequence has been shown to be closest to the consensus clade B sequence with only 16 nonidentical residues out of 848 (Korber, et al., 1998, Human retroviruses and AIDS, Los Alamos National Laboratory, Los Alamos, N.M.). The skilled artisan will understand after review of this specification that any available HIV-1 or HIV-2 strain provides a potential template for the generation of HIV pol DNA vaccine constructs disclosed herein. It is further noted that the protease gene is excluded from the DNA vaccine constructs of the present invention to insure safety from any residual protease activity in spite of mutational inactivation. The design of the gene sequences for both wild-type (wt-pol) and inactivated pol (IA-pol) incorporates the use of human preferred (“humanize”) codons for each amino acid residue in the sequence in order to maximize in vivo mammalian expression (Lathe, 1985, J. Mol. Biol. 183:1-12). As can be discerned by inspecting the codon usage in SEQ ID NOs: 1, 3, 5 and 7, the following codon usage for mammalian optimization is preferred: Met (ATG), Gly (GGC), Lys (AAG), Trp (TGG), Ser (TCC), Arg (AGG), Val (GTG), Pro (CCC), Thr (ACC), Glu (GAG); Leu (CTG), His (CAC), ie (ATC), Asn (AAC), Cys (TGC), Ala (GCC), Gln (CAG), Phe (TTC) and Tyr (TAC). For an additional discussion relating to mammalian (human) codon optimization, see WO 97/31115 (PCT/US97/02294), which, as noted elsewhere in this specification, is hereby incorporated by reference. It is intended that the skilled artisan may use alternative versions of codon optimization or may omit this step when generating HIV pol vaccine constructs within the scope of the present invention. Therefore, the present invention also relates to non-codon optimized versions of DNA molecules and associated recombinant adenoviral HIV vaccines which encode the various wild type and modified forms of the HIV Pol protein disclosed herein. However, codon optimization of these constructs is a preferred embodiment of this invention.

A particular embodiment of this portion of the invention comprisies codon optimized nucleotide sequences which encode wt-pol DNA constructs (herein, “wt-pol” or “wt-pol (codon optimized))” wherein DNA sequences encoding the protease (PR) activity are deleted, leaving codon optimized “wild type” sequences which encode RT (reverse transcriptase and RNase H activity) and IN integrase activity. A DNA molecule which encodes this protein is disclosed herein as SEQ ID NO: 1, the open reading frame being contained from an initiating Met residue at nucleotides 10-12 to a termination codon from nucleotides 2560-2562. SEQ ID NO: 1 is as follows:

(SEQ ID NO:1)

AGATCTACCA TGGCCCCCAT CTCCCCCATT GAGACTGTGC
CTGTGAAGCT GAAGCCTGGC ATGGATGGCC CCAAGGTGAA
GCAGTGGCCC CTGACTGAGG AGAAGATCAA GGCCCTGGTG
GAAATCTGCA CTGAGATGGA GAAGGAGGGC AAAATCTCCA
AGATTGGCCC CGAGAACCCC TACAACACCC CTGTGTTTGC
CATCAAGAAG AAGGACTCCA CCAAGTGGAG GAAGCTGGTG
GACTTCAGGG AGCTGAACAA GAGGACCCAG GACTTCTGGG
AGGTGCAGCT GGGCATCCCC CACCCCGCTG GCCTGAAGAA
GAAGAAGTCT GTGACTGTGC TGGATGTGGG GGATGCCTAC
TTCTCTGTGC CCCTGGATGA GGACTTCAGG AAGTACACTG
CCTTCACCAT CCCCTCCATC AACAATGAGA CCCCTGGCAT
CAGGTACCAG TACAATGTGC TGCCCCAGGG CTGGAAGGGC
TCCCCTGCCA TCTTCCAGTC CTCCATGACC AAGATCCTGG
AGCCCTTCAG GAAGCAGAAC CCTGACATTG TGATCTACCA
GTACATGGAT GACCTGTATG TGGGCTCTGA CCTGGAGATT
GGGCAGCACA GGACCAAGAT TGAGGAGCTG AGGCAGCACC
TGCTGAGGTG GGGCCTGACC ACCCCTGACA AGAAGCACCA
GAAGGAGCCC CCCTTCCTGT GGATGGGCTA TGAGCTGCAC
CCCGACAAGT GGACTGTGCA GCCCATTGTG CTGCCTGAGA
AGGACTCCTG GACTGTGAAT GACATCCAGA AGCTGGTGGG
CAAGCTGAAC TGGGCCTCCC AAATCTACCC TGGCATCAAG
GTGAGGCAGC TGTGCAAGCT GCTGAGGGGC ACCAAGGCCC
TGACTGAGGT GATCCCCCTG ACTGAGGAGG CTGAGCTGGA
GCTGGCTGAG AACAGGGAGA TCCTGAAGGA GCCTGTGCAT
GGGGTGTACT ATGACCCCTC CAAGGACCTG ATTGCTGAGA
TCCAGAAGCA GGGCCAGGGC CAGTGGACCT ACCAAATCTA
CCAGGAGCCC TTCAAGAACC TGAAGACTGG CAAGTATGCC
AGGATGAGGG GGGCCCACAC CAATGATGTG AAGCAGCTGA
CTGAGGCTGT GCAGAAGATC ACCACTGAGT CCATTGTGAT
CTGGGGCAAG ACCCCCAAGT TCAAGCTGCC CATCCAGAAG
GAGACCTGGG AGACCTGGTG GACTGAGTAC TGGCAGGCCA
CCTGGATCCC TGAGTGGGAG TTTGTGAACA CCCCCCCCCT
GGTGAAGCTG TGGTACCAGC TGGAGAAGGA GCCCATTGTG
GGGGCTGAGA CCTTCTATGT GGATGGGGCT GCCAACAGGG
AGACCAAGCT GGGCAAGGCT GGCTATGTGA CCAACAGGGG
CAGGCAGAAG GTGGTGACCC TGACTGACAC CACCAACCAG
AAGACTGAGC TCCAGGCCAT CTACCTGGCC CTCCAGGACT
CTGGCCTGGA GGTGAACATT GTGACTGACT CCCAGTATGC
CCTGGGCATC ATCCAGGCCC AGCCTGATCA GTCTGAGTCT
GAGCTGGTGA ACCAGATCAT TGAGCAGCTG ATCAAGAAGG
AGAAGGTGTA CCTGGCCTGG GTGCCTGCCC ACAAGGGCAT
TGGGGGCAAT GAGCAGGTGG ACAAGCTGGT GTCTGCTGGC
ATCAGGAAGG TGCTGTTCCT GGATGGCATT GACAAGGCCC
AGGATGAGCA TGAGAAGTAC CACTCCAACT GGAGGGCTAT
GGCCTCTGAC TTCAACCTGC CCCCTGTGGT GGCTAAGGAG
ATTGTGGCCT CCTGTGACAA GTGCCAGCTG AAGGGGGAGG
CCATGCATGG GCAGGTGGAC TGCTCCCCTG GCATCTGGCA
GCTGGACTGC ACCCACCTGG AGGGCAAGGT GATCCTGGTG
GCTGTGCATG TGGCCTCCGG CTACATTGAG GCTGAGGTGA
TCCCTGCTGA GACAGGCCAG GAGACTGCCT ACTTCCTGCT
GAAGCTGGCT GGCAGGTGGC CTGTGAAGAC CATCCACACT
GACAATGGCT CCAACTTCAC TGGGGCCACA GTGAGGGCTG
CCTGCTGGTG GGCTGGCATC AAGCAGGAGT TTGGCATCCC
CTACAACCCC CAGTCCCAGG GGGTGGTGGA GTCCATGAAC
AAGGAGCTGA AGAAGATCAT TGGGCAGGTG AGGGACCAGG
CTGAGCACCT GAAGACAGCT GTGCAGATGG CTGTGTTCAT
CCACAACTTC AAGAGGAAGG GGGGCATCGG GGGCTACTCC
GCTGGGGAGA GGATTGTGGA CATCATTGCC ACAGACATCC
AGACCAAGGA GCTCCAGAAG CAGATCACCA AGATCCAGAA
CTTCAGGGTG TACTACAGGG ACTCCAGGAA CCCCCTGTGG
AAGGGCCCTG CCAAGCTGCT GTGGAAGGGG GAGGGGGCTG
TGGTGATCCA GGACAACTCT GACATCAAGG TGGTGCCCAG
GAGGAAGGCC AAGATCATCA GGGACTATGG CAAGCAGATG
GCTCGGGATG ACTGTGTGGC CTCCAGGCAG GATGAGGACT
AAAGCCCGGG CAGATCT.

The open reading frame of the wild type pol construct disclosed as SEQ ID NO:1 contains 850 amino acids, disclosed herein as SEQ ID NO:2, as follows:

(SEQ ID NO:2)

Met Ala Pro Ile Ser Pro Ile Glu Thr Val Pro Val
Lys Leu Lys Pro Gly Met Asp Gly Pro Lys Val Lys
Gln Trp Pro Leu Thr Glu Glu Lys Ile Lys Ala Leu
Val Glu Ile Cys Thr Glu Met Glu Lys Glu Gly Lys
Ile Ser Lys Ile Gly Pro Glu Asn Pro Tyr Asn Thr
Pro Val Phe Ala Ile Lys Lys Lys Asp Ser Thr Lys
Trp Arg Lys Leu Val Asp Phe Arg Glu Leu Asn Lys
Arg Thr Gln Asp Phe Trp Glu Val Gln Leu Gly Ile
Pro His Pro Ala Gly Leu Lys Lys Lys Lys Ser Val
Thr Val Leu Asp Val Gly Asp Ala Tyr Phe Ser Val
Pro Leu Asp Glu Asp Phe Arg Lys Tyr Thr Ala Phe
Thr Ile Pro Ser Ile Asn Asn Glu Thr Pro Gly Ile
Arg Tyr Gln Tyr Asn Val Leu Pro Gln Gly Trp Lys
Gly Ser Pro Ala Ile Phe Gln Ser Ser Met Thr Lys
Ile Leu Glu Pro Phe Arg Lys Gln Asn Pro Asp Ile
Val Ile Tyr Gln Tyr Met Asp Asp Leu Tyr Val Gly
Ser Asp Leu Glu Ile Gly Gln His Arg Thr Lys Ile
Glu Glu Leu Arg Gln His Leu Leu Arg Trp Gly Leu
Thr Thr Pro Asp Lys Lys His Gln Lys Glu Pro Pro
Phe Leu Trp Met Gly Tyr Glu Leu His Pro Asp Lys
Trp Thr Val Gln Pro Ile Val Leu Pro Glu Lys Asp
Ser Trp Thr Val Asn Asp Ile Gln Lys Leu Val Gly
Lys Leu Asn Trp Ala Ser Gln Ile Tyr Pro Gly Ile
Lys Val Arg Gln Leu Cys Lys Leu Leu Arg Gly Thr
Lys Ala Leu Thr Glu Val Ile Pro Leu Thr Glu Glu
Ala Glu Leu Glu Leu Ala Glu Asn Arg Glu Ile Leu
Lys Glu Pro Val His Gly Val Tyr Tyr Asp Pro Ser
Lys Asp Leu Ile Ala Glu Ile Gln Lys Gln Gly Gln
Gly Gln Trp Thr Tyr Gln Ile Tyr Gln Glu Pro Phe
Lys Asn Leu Lys Thr Gly Lys Tyr Ala Arg Met Arg
Gly Ala His Thr Asn Asp Val Lys Gln Leu Thr Glu
Ala Val Gln Lys Ile Thr Thr Glu Ser Ile Val Ile
Trp Gly Lys Thr Pro Lys Phe Lys Leu Pro Ile Gln
Lys Glu Thr Trp Glu Thr Trp Trp Thr Glu Tyr Trp
Gln Ala Thr Trp Ile Pro Glu Trp Glu Phe Val Asn
Thr Pro Pro Leu Val Lys Leu Trp Tyr Gln Leu Glu
Lys Glu Pro Ile Val Gly Ala Glu Thr Phe Tyr Val
Asp Gly Ala Ala Asn Arg Glu Thr Lys Leu Gly Lys
Ala Gly Tyr Val Thr Asn Arg Gly Arg Gln Lys Val
Val Thr Leu Thr Asp Thr Thr Asn Gln Lys Thr Glu
Leu Gln Ala Ile Tyr Leu Ala Leu Gln Asp Ser Gly
Leu Glu Val Asn Ile Val Thr Asp Ser Gln Tyr Ala
Leu Gly Ile Ile Gln Ala Gln Pro Asp Gln Ser Glu
Ser Glu Leu Val Asn Gln Ile Ile Glu Gln Leu Ile
Lys Lys Glu Lys Val Tyr Leu Ala Trp Val Pro Ala
His Lys Gly Ile Gly Gly Asn Glu Gln Val Asp Lys
Leu Val Ser Ala Gly Ile Arg Lys Val Leu Phe Leu
Asp Gly Ile Asp Lys Ala Gln Asp Glu His Glu Lys
Tyr His Ser Asn Trp Arg Ala Met Ala Ser Asp Phe
Asn Leu Pro Pro Val Val Ala Lys Glu Ile Val Ala
Ser Cys Asp Lys Cys Gln Leu Lys Gly Glu Ala Met
His Gly Gln Val Asp Cys Ser Pro Gly Ile Trp Gln
Leu Asp Cys Thr His Leu Glu Gly Lys Val Ile Leu
Val Ala Val His Val Ala Ser Gly Tyr Ile Glu Ala
Glu Val Ile Pro Ala Glu Thr Gly Gln Glu Thr Ala
Tyr Phe Leu Leu Lys Leu Ala Gly Arg Trp Pro Val
Lys Thr Ile His Thr Asp Asn Gly Ser Asn Phe Thr
Gly Ala Thr Val Arg Ala Ala Cys Trp Trp Ala Gly
Ile Lys Gln Glu Phe Gly Ile Pro Tyr Asn Pro Gln
Ser Gln Gly Val Val Glu Ser Met Asn Lys Glu Leu
Lys Lys Ile Ile Gly Gln Val Arg Asp Gln Ala Glu
His Leu Lys Thr Ala Val Gln Met Ala Val Phe Ile
His Asn Phe Lys Arg Lys Gly Gly Ile Gly Gly Tyr
Ser Ala Gly Glu Arg Ile Val Asp Ile Ile Ala Thr
Asp Ile Gln Thr Lys Glu Leu Gln Lys Gln Ile Thr
Lys Ile Gln Asn Phe Arg Val Tyr Tyr Arg Asp Ser
Arg Asn Pro Leu Trp Lys Gly Pro Ala Lys Leu Leu
Trp Lys Gly Glu Gly Ala Val Val Ile Gln Asp Asn
Ser Asp Ile Lys Val Val Pro Arg Arg Lys Ala Lys
Ile Ile Arg Asp Tyr Gly Lys Gln Met Ala Gly Asp
Asp Cys Val Ala Ser Arg Gln Asp Glu Asp.

The present invention especially relates to an adenoviral vector vaccine which comprises a codon optimized HIV-1 DNA pol construct wherein, in addition to deletion of the portion of the wild type sequence encoding the protease activity, a combination of active site residue mutations are introduced which are deleterious to HIV-1 pol (RT-RH-IN) activity of the expressed protein. Therefore, the present invention preferably relates to an adenoviral HIV-1 DNA pol-based vaccine wherein the construct is devoid of DNA sequences encoding any PR activity, as well as containing a mutation(s) which at least partially, and preferably substantially, abolishes RT, RNase and/or IN activity. One type of HIV-1 pol mutant which is part and parcel of an adenoviral vector vaccine may include but is not limited to a mutated DNA molecule comprising at least one nucleotide substitution which results in a point mutation which effectively alters an active site within the RT, RNase and/or IN regions of the expressed protein, resulting in at least substantially decreased enzymatic activity for the RT, RNase H and/or IN functions of HIV-1 Pol. In a preferred embodiment of this portion of the invention, a HIV-1 DNA pol construct contains a mutation or mutations within the Pol coding region which effectively abolishes RT, RNase H and IN activity. An especially preferable HIV-2 DNA pol construct in a DNA molecule which contains at least one point mutation which alters the active site of the RT, RNase H and IN domains of Pol, such that each activity is at least substantially abolished. Such a HIV-1 Pol mutant will most likely comprise at least one point mutation in or around each catalytic domain responsible for RT, RNase H and IN activity, respectfully. To this end, an especially preferred HIV-1 DNA pol construct is exemplified herein and contains nine codon substitution mutations which results in an inactivated Pol protein (IA Pol: SEQ ID NO:4, FIGS. 17A-1 to 17A-3) which has no PR, RT, RNase or IN activity, wherein three such point mutations reside within each of the RT, RNase and IN catalytic domains. Therefore, an especially preferred exemplification is an adenoviral vaccine which comprises, in an appropriate fashion, a DNA molecule which encodes IA-pol, which contains all nine mutations as shown below in Table 1. An additional preferred amino acid residue for substitution is Asp551, localized within the RNase domain of Pol. Any combination of the mutations disclosed herein may suitable and therefore may be utilized as an IA-Pol-based vaccine of the present invention. While addition and deletion mutations are contemplated and within the scope of the invention, the preferred mutation is a point mutation resulting in a substitution of the wild type amino acid with an alternative amino acid residue.

TABLE 1

| wt aa | aa residue | mutant aa | enzyme function |
|---|---|---|---|
| Asp | 112 | Ala | RT |
| Asp | 187 | Ala | RT |
| Asp | 188 | Ala | RT |
| Asp | 445 | Ala | RNase H |
| Glu | 480 | Ala | RNase H |
| Asp | 500 | Ala | RNase H |
| Asp | 626 | Ala | IN |
| Asp | 678 | Ala | IN |
| Glu | 714 | Ala | IN |

It is preferred that point mutations be incorporated into the IApol mutant adenoviral vaccines of the present invention so as to lessen the possibility of altering epitopes in and around the active site(s) of HIV-1 Pol.

To this end, SEQ ID NO:3 discloses the nucleotide sequence which codes for a codon optimized pol in addition to the nine mutations shown in Table 1, disclosed as follows, and referred to herein as “IApol”:

(SEQ ID NO:3)

AGATCTACCA TGGCCCCCAT CTCCCCCATT GAGACTGTGC
CTGTGAAGCT GAAGCCTGGC ATGGATGGCC CCAAGGTGAA
GCAGTGGCCC CTGACTGAGG AGAAGATCAA GGCCCTGGTG
GAAATCTGCA CTGAGATGGA GAAGGAGGGC AAAATCTCCA
AGATTGGCCC CGAGAACCCC TACAACACCC CTGTGTTTGC
CATCAAGAAG AAGGACTCCA CCAAGTGGAG GAAGCTGGTG
GACTTCAGGG AGCTGAACAA GAGGACCCAG GACTTCTGGG
AGGTGCAGCT GGGCATCCCC CACCCCGCTG GCCTGAAGAA
GAAGAAGTCT GTGACTGTGC TGGCTGTGGG GGATGCCTAC
TTCTCTGTGC CCCTGGATGA GGACTTCAGG AAGTACACTG
CCTTCACCAT CCCCTCCATC AACAATGAGA CCCCTGGCAT
CAGGTACCAG TACAATGTGC TGCCCCAGGG CTGGAAGGGC
TCCCCTGCCA TCTTCCAGTC CTCCATGACC AAGATCCTGG
AGCCCTTCAG GAAGCAGAAC CCTGACATTG TGATCTACCA
GTACATGGCT GCCCTGTATG TGGGCTCTGA CCTGGAGATT
GGGCAGCACA GGACCAAGAT TGAGGAGCTG AGCCAGCACC
TGCTGAGGTG GGGCCTGACC ACCCCTGACA AGAAGCACCA
GAAGGAGCCC CCCTTCCTGT GGATGGGCTA TGAGCTGCAC
CCCGACAAGT GGACTGTGCA GCCCATTGTG CTGCCTGAGA
AGGACTCCTG GACTGTGAAT GACATCCAGA AGCTGGTGGG
CAAGCTGAAC TGGGCCTCCC AAATCTACCC TGGCATCAAG
GTGAGGCAGC TGTGCAAGCT GCTGAGGGGC ACCAAGGCCC
TGACTGAGGT GATCCCCCTG ACTGAGGAGG CTGAGCTGGA
GCTGGCTGAG AACAGGGAGA TCCTGAAGGA GCCTGTGCAT
GGGGTGTACT ATGACCCCTC CAAGGACCTG ATTGCTGAGA
TCCAGAAGCA GGGCCAGGGC CAGTGGACCT ACCAAATCTA
CCAGGAGCCC TTCAAGAACC TGAAGACTGG CAAGTATGCC
AGGATGAGGG GGGCCCACAC CAATGATGTG AAGCAGCTGA
CTGAGGCTGT GCAGAAGATC ACCACTGAGT CCATTGTGAT
CTGGGGCAAG ACCCCCAAGT TCAAGCTGCC CATCCAGAAG
GAGACCTGGG AGACCTGGTG GACTGAGTAC TGGCAGGCCA
CCTGGATCCC TGAGTGGGAG TTTGTGAACA CCCCCCCCCT
GGTGAAGCTG TGGTACCAGC TGGAGAAGGA GCCCATTGTG
GGGGCTGAGA CCTTCTATGT GGCTGGGGCT GCCAACAGGG
AGACCAAGCT GGGCAAGGCT GGCTATGTGA CCAACAGGGG
CAGGCAGAAG GTGGTGACCC TGACTGACAC CACCAACCAG
AAGACTGCCC TCCAGGCCAT CTACCTGGCC CTCCAGGACT
CTGGCCTGGA GGTGAACATT GTGACTGCCT CCCAGTATGC
CCTGGGCATC ATCCAGGCCC AGCCTGATCA GTCTGAGTCT
GAGCTGGTGA ACCAGATCAT TGAGCAGCTG ATCAAGAAGG
AGAAGGTGTA CCTGGCCTGG GTGCCTGCCC ACAAGGGCAT
TGGGGGCAAT GAGCAGGTGG ACAAGCTGGT GTCTGCTGGC
ATCAGGAAGG TGCTGTTCCT GGATGGCATT GACAAGGCCC
AGGATGAGCA TGAGAAGTAC CACTCCAACT GGAGGGCTAT
GGCCTCTGAC TTCAACCTGC CCCCTGTGGT GGCTAAGGAG
ATTGTGGCCT CCTGTGACAA GTGCCAGCTG AAGGGGGAGG
CCATGCATGG GCAGGTGGAC TGCTCCCCTG GCATCTGGCA
GCTGGCCTGC ACCCACCTGG AGGGCAAGGT GATCCTGGTG
GCTGTGCATG TGGCCTCCGG CTACATTGAG GCTGAGGTGA
TCCCTGCTGA GACAGGCCAG GAGACTGCCT ACTTCCTGCT
GAAGCTGGCT GGCAGGTGGC CTGTGAAGAC CATCCACACT
GCCAATGGCT CCAACTTCAC TGGGGCCACA GTGAGGGCTG
CCTGCTGGTG GGCTGGCATC AAGCAGGAGT TTGGCATCCC
CTACAACCCC CAGTCCCAGG GGGTGGTGGC CTCCATGAAC
AAGGAGCTGA AGAAGATCAT TGGGCAGGTG AGGGACCAGG
CTGAGCACCT GAAGACAGCT GTGCAGATGG CTGTGTTCAT
CCACAACTTC AAGAGGAAGG GGGGCATCGG GGGCTACTCC
GCTGGGGAGA GGATTGTGGA CATCATTGCC ACAGACATCC
AGACCAAGGA GCTCCAGAAG CAGATCACCA AGATCCAGAA
CTTCAGGGTG TACTACAGGG ACTCCAGGAA CCCCCTGTGG
AAGGGCCCTG CCAAGCTGCT GTGGAAGGGG GAGGGGGCTG
TGGTGATCCA GGACAACTCT GACATCAAGG TGGTGCCCAG
GAGGAAGGCC AAGATCATCA GGGACTATGG CAAGCAGATG
GCTGGGGATG ACTGTGTGGC CTCCAGGCAG GATGAGGACT
AAAGCCCGGG CAGATCT.

In order to produce the IA-pol-based adenoviral vaccines of the present invention, inactivation of the enzymatic functions was achieved by replacing a total of nine active site residues from the enzyme subunits with alanine side-chains. As shown in Table 1, all residues that comprise the catalytic triad of the polymerase, namely Asp112, Asp187, and Asp188, were substituted with alanine (Ala) residues (Larder, et al., Nature 1987, 327: 716-717; Larder, et al., 1989, Proc. Natl. Acad. Sci. 1989, 86: 4803-4807). Three additional mutations were introduced at Asp445, Glu480 and Asp500 to abolish RNase H activity (Asp551 was left unchanged in this IA Pol construct), with each residue being substituted for an Ala residue, respectively (Davies, et al., 1991, Science 252:, 88-95; Schatz, et al., 1989, FEBS Lett. 257: 311-314; Mizrahi, et al., 1990, Nucl. Acids. Res. 18: pp. 5359-5353). HIV pol integrase function was abolished through three mutations at Asp626, Asp678 and Glu714. Again, each of these residues has been substituted with an Ala residue (Wiskerchen, et al., 1995, J. Virol. 69: 376-386; Leavitt, et al., 1993, J. Biol. Chem. 268: 2113-2119). Amino acid residue Pro3 of SEQ ID NO:4 marks the start of the RT gene. The complete amino acid sequence of IA-Pol is disclosed herein as SEQ ID NO:4 and FIGS. 17A-1 to 17A-3, as follows:

(SEQ ID NO:4)

Met Ala Pro Ile Ser Pro Ile Glu Thr Val Pro Val
Lys Leu Lys Pro Gly Met Asp Gly Pro Lys Val Lys
Gln Trp Pro Leu Thr Glu Glu Lys Ile Lys Ala Leu
Val Glu Ile Cys Thr Glu Met Glu Lys Glu Gly Lys
Ile Ser Lys Ile Gly Pro Glu Asn Pro Tyr Asn Thr
Pro Val Phe Ala Ile Lys Lys Lys Asp Ser Thr Lys
Trp Arg Lys Leu Val Asp Phe Arg Glu Leu Asn Lys
Arg Thr Gln Asp Phe Trp Glu Val Gln Leu Gly Ile
Pro His Pro Ala Gly Leu Lys Lys Lys Lys Ser Val
Thr Val Leu Ala Val Gly Asp Ala Tyr Phe Ser Val
Pro Leu Asp Glu Asp Phe Arg Lys Tyr Thr Ala Phe
Thr Ile Pro Ser Ile Asn Asn Glu Thr Pro Gly Ile
Arg Tyr Gln Tyr Asn Val Leu Pro Gln Gly Trp Lys
Gly Ser Pro Ala Ile Phe Gln Ser Ser Met Thr Lys
Ile Leu Glu Pro Phe Arg Lys Gln Asn Pro Asp Ile
Val Ile Tyr Gln Tyr Met Ala Ala Leu Tyr Val Gly
Ser Asp Leu Glu Ile Gly Gln His Arg Thr Lys Ile
Glu Glu Leu Arg Gln His Leu Leu Arg Trp Gly Leu
Thr Thr Pro Asp Lys Lys His Gln Lys Glu Pro Pro
Phe Leu Trp Met Gly Tyr Glu Leu His Pro Asp Lys
Trp Thr Val Gln Pro Ile Val Leu Pro Glu Lys Asp
Ser Trp Thr Val Asn Asp Ile Gln Lys Leu Val Gly
Lys Leu Asn Trp Ala Ser Gln Ile Tyr Pro Gly Ile
Lys Val Arg Gln Leu Cys Lys Leu Leu Arg Gly Thr
Lys Ala Leu Thr Glu Val Ile Pro Leu Thr Glu Glu
Ala Glu Leu Glu Leu Ala Glu Asn Arg Glu Ile Leu
Lys Glu Pro Val His Gly Val Tyr Tyr Asp Pro Ser
Lys Asp Leu Ile Ala Glu Ile Gln Lys Gln Gly Gln
Gly Gln Trp Thr Tyr Gln Ile Tyr Gln Glu Pro Phe
Lys Asn Leu Lys Thr Gly Lys Tyr Ala Arg Met Arg
Gly Ala His Thr Asn Asp Val Lys Gln Leu Thr Glu
Ala Val Gln Lys Ile Thr Thr Glu Ser Ile Val Ile
Trp Gly Lys Thr Pro Lys Phe Lys Leu Pro Ile Gln
Lys Glu Thr Trp Glu Thr Trp Trp Thr Glu Tyr Trp
Gln Ala Thr Trp Ile Pro Glu Trp Glu Phe Val Asn
Thr Pro Pro Leu Val Lys Leu Trp Tyr Gln Leu Glu
Lys Glu Pro Ile Val Gly Ala Glu Thr Phe Tyr Val
Ala Gly Ala Ala Asn Arg Glu Thr Lys Leu Gly Lys
Ala Gly Tyr Val Thr Asn Arg Gly Arg Gln Lys Val
Val Thr Leu Thr Asp Thr Thr Asn Gln Lys Thr Ala
Leu Gln Ala Ile Tyr Leu Ala Leu Gln Asp Ser Gly
Leu Glu Val Asn Ile Val Thr Ala Ser Gln Tyr Ala
Leu Gly Ile Ile Gln Ala Gln Pro Asp Gln Ser Glu
Ser Glu Leu Val Asn Gln Ile Ile Glu Gln Leu Ile
Lys Lys Glu Lys Val Tyr Leu Ala Trp Val Pro Ala
His Lys Gly Ile Gly Gly Asn Glu Gln Val Asp Lys
Leu Val Ser Ala Gly Ile Arg Lys Val Leu Phe Leu
Asp Gly Ile Asp Lys Ala Gln Asp Glu His Glu Lys
Tyr His Ser Asn Trp Arg Ala Met Ala Ser Asp Phe
Asn Leu Pro Pro Val Val Ala Lys Glu Ile Val Ala
Ser Cys Asp Lys Cys Gln Leu Lys Gly Glu Ala Met
His Gly Gln Val Asp Cys Ser Pro Gly Ile Trp Gln
Leu Ala Cys Thr His Leu Glu Gly Lys Val Ile Leu
Val Ala Val His Val Ala Ser Gly Tyr Ile Glu Ala
Glu Val Ile Pro Ala Glu Thr Gly Gln Glu Thr Ala
Tyr Phe Leu Leu Lys Leu Ala Gly Arg Trp Pro Val
Lys Thr Ile His Thr Ala Asn Gly Ser Asn Phe Thr
Gly Ala Thr Val Arg Ala Ala Cys Trp Trp Ala Gly
Ile Lys Gln Glu Phe Gly Ile Pro Tyr Asn Pro Gln
Ser Gln Gly Val Val Ala Ser Met Asn Lys Glu Leu
Lys Lys Ile Ile Gly Gln Val Arg Asp Gln Ala Glu
His Leu Lys Thr Ala Val Gln Met Ala Val Phe Ile
His Asn Phe Lys Arg Lys Gly Gly Ile Gly Gly Tyr
Ser Ala Gly Glu Arg Ile Val Asp Ile Ile Ala Thr
Asp Ile Gln Thr Lys Glu Leu Gln Lys Gln Ile Thr
Lys Ile Gln Asn Phe Arg Val Tyr Tyr Arg Asp Ser
Arg Asn Pro Leu Trp Lys Gly Pro Ala Lys Leu Leu
Trp Lys Gly Glu Gly Ala Val Val Ile Gln Asp Asn
Ser Asp Ile Lys Val Val Pro Arg Arg Lys Ala Lys
Ile Ile Arg Asp Tyr Gly Lys Gln Met Ala Gly Asp
Asp Cys Val Ala Ser Arg Gln Asp Glu Asp.

As noted above, it will be understood that any combination of the mutations disclosed above may be suitable and therefore be utilized as an IA-pol-based adenoviral HIV vaccine of the present invention, either when administered alone or in a combined modality regime and/or a prime-boost regimen. For example, it may be possible to mutate only 2 of the 3 residues within the respective reverse transcriptase, RNase H, and integrase coding regions while still abolishing these enzymatic activities. However, the IA-pol construct described above and disclosed as SEQ ID NO:3, as well as the expressed protein (SEQ ID NO:4;) is preferred. It is also preferred that at least one mutation be present in each of the three catalytic domains.

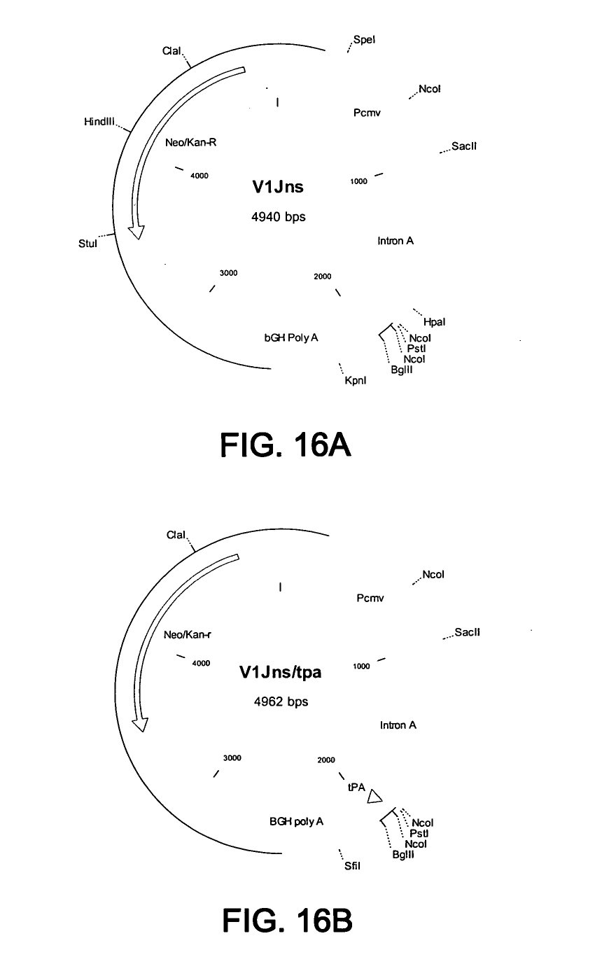

Another aspect of this portion of the invention are codon optimized HIV-1 Pol-based vaccine constructions which comprise a eukaryotic trafficking signal peptide such as from tPA (tissue-type plasminogen activator) or by a leader peptide such as is found in highly expressed mammalian proteins such as immunoglobulin leader peptides. Any functional leader peptide may be tested for efficacy. However, a preferred embodiment of the present invention, as with HIV-1 Nef constructs shown herein, is to provide for a HIV-1 Pol mutant adenoviral vaccine construction wherein the pol coding region or a portion thereof is operatively linked to a leader peptide, preferably a leader peptide from human tPA. In other words, a codon optimized HIV-1 Pol mutant such as IA-Pol (SEQ ID NO:4) may also comprise a leader peptide at the amino terminal portion of the protein, which may effect cellular trafficking and hence, immunogenicity of the expressed protein within the host cell. As noted in FIG. 16A-B, a DNA vector which may be utilized to practice the present invention may be modified by known recombinant DNA methodology to contain a leader signal peptide of interest, such that downstream cloning of the modified HIV-1 protein of interest results in a nucleotide sequence which encodes a modified HIV-1 tPA/Pol protein. In the alternative, as noted above, insertion of a nucleotide sequence which encodes a leader peptide may be inserted into a DNA vector housing the open reading frame for the Pol protein of interest. Regardless of the cloning strategy, the end result is a polynucleotide vaccine which comprises vector components for effective gene expression in conjunction with nucleotide sequences which encode a modified HIV-1 Pol protein of interest, including but not limited to a HIV-1 Pol protein which contains a leader peptide. The amino acid sequence of the human tPA leader utilized herein is as follows: MDAMKRGLCCVLLLCGAVFVSPSEISS (SEQ ID NO:17). Therefore, another aspect of the present invention is to generate HIV-1Pol-based vaccine constructions which comprise a eukaryotic trafficking signal peptide such as from tPA. To this end, the present invention relates to a DNA molecule which encodes a codon optimized wt-pol DNA construct wherein the protease (PR) activity is deleted and a human tPA leader sequence is fused to the 5′ end of the coding region. A DNA molecule which encodes this protein is disclosed herein as SEQ ID NO:5, the open reading frame disclosed herein as SEQ ID NO:6.

To this end, the present invention relates to a DNA molecule which encodes a codon optimized wt-pol DNA construct wherein the protease (PR) activity is deleted and a human tPA leader sequence is fused to the 5′ end of the coding region (herein, “tPA-wt-pol”). A DNA molecule which encodes this protein is disclosed herein as SEQ ID NO:5, the open reading frame being contained from an initiating Met residue at nucleotides 8-10 to a termination codon from nucleotides 2633-2635. SEQ ID NO:5 is as follows:

(SEQ ID NO:5)

GATCACCATG GATGCAATGA AGAGAGGGCT CTGCTGTGTG
CTGCTGCTGT GTGGAGCAGT CTTCGTTTCG CCCAGCGAGA
TCTCCGCCCC CATCTCCCCC ATTGAGACTG TGCCTGTGAA
GCTGAAGCCT GGCATGGATG GCCCCAAGGT GAAGCAGTGG
CCCCTGACTG AGGAGAAGAT CAAGGCCCTG GTGGAAATCT
GCACTGAGAT GGAGAAGGAG GGCAAAATCT CCAAGATTGG
CCCCGAGAAC CCCTACAACA CCCCTGTGTT TGCCATCAAG
AAGAAGGACT CCACCAAGTG GAGGAAGCTG GTGGACTTCA
GGGAGCTGAA CAAGAGGACC CAGGACTTCT GGGAGGTGCA
GCTGGGCATC CCCCACCCCG CTGGCCTGAA GAAGAAGAAG
TCTGTGACTG TGCTGGATGT GGGGGATGCC TACTTCTCTG
TGCCCCTGGA TGAGGACTTC AGGAAGTACA CTGCCTTCAC
CATCCCCTCC ATCAACAATG AGACCCCTGG CATCAGGTAC
CAGTACAATG TGCTGCCCCA GGGCTGGAAG GGCTCCCCTG
CCATCTTCCA GTCCTCCATG ACCAAGATCC TGGAGCCCTT
CAGGAAGCAG AACCCTGACA TTGTGATCTA CCAGTACATG
GATGACCTGT ATGTGGGCTC TGACCTGGAG ATTGGGCAGC
ACAGGACCAA GATTGAGGAG CTGAGGCAGC ACCTGCTGAG
GTGGGGCCTG ACCACCCCTG ACAAGAAGCA CCAGAAGGAG
CCCCCCTTCC TGTGGATGGG CTATGAGCTG CACCCCGACA
AGTGGACTGT GCAGCCCATT GTGCTGCCTG AGAAGGACTC
CTGGACTGTG AATGACATCC AGAAGCTGGT GGGCAAGCTG
AACTGGGCCT CCCAAATCTA CCCTGGCATC AAGGTGAGGC
AGCTGTGCAA GCTGCTGAGG GGCACCAAGG CCCTGACTGA
GGTGATCCCC CTGACTGAGG AGGCTGAGCT GGAGCTGGCT
GAGAACAGGG AGATCCTGAA GGAGCCTGTG CATGGGGTGT
ACTATGACCC CTCCAAGGAC CTGATTGCTG AGATCCAGAA
GCAGGGCCAG GGCCAGTGGA CCTACCAAAT CTACCAGGAG
CCCTTCAAGA ACCTGAAGAC TGGCAAGTAT GCCAGGATGA
GGGGGGCCCA CACCAATGAT GTGAAGCAGC TGACTGAGGC
TGTGCAGAAG ATCACCACTG AGTCCATTGT GATCTGGGGC
AAGACCCCCA AGTTCAAGCT GCCCATCCAG AAGGAGACCT
GGGAGACCTG GTGGACTGAG TACTGGCAGG CCACCTGGAT
CCCTGAGTGG GAGTTTGTGA ACACCCCCCC CCTGGTGAAG
CTGTGGTACC AGCTGGAGAA GGAGCCCATT GTGGGGGCTG
AGACCTTCTA TGTGGATGGG GCTGCCAACA GGGAGACCAA
GCTGGGCAAG GCTGGCTATG TGACCAACAG GGGCAGGCAG
AAGGTGGTGA CCCTGACTGA CACCACCAAC CAGAAGACTG
AGCTCCAGGC CATCTACCTG GCCCTCCAGG ACTCTGGCCT
GGAGGTGAAC ATTGTGACTG ACTCCCAGTA TGCCCTGGGC
ATCATCCAGG CCCAGCCTGA TCAGTCTGAG TCTGAGCTGG
TGAACCAGAT CATTGAGCAG CTGATCAAGA AGGAGAAGGT
GTACCTGGCC TGGGTGCCTG CCCACAAGGG CATTGGGGGC
AATGAGCAGG TGGACAAGCT GGTGTCTGCT GGCATCAGGA
AGGTGCTGTT CCTGGATGGC ATTGACAAGG CCCAGGATGA
GCATGAGAAG TACCACTCCA ACTGGAGGGC TATGGCCTCT
GACTTCAACC TGCCCCCTGT GGTGGCTAAG GAGATTGTGG
CCTCCTGTGA CAAGTGCCAG CTGAAGGGGG AGGCCATGCA
TGGGCAGGTG GACTGCTCCC CTGGCATCTG GCAGCTGGAC
TGCACCCACC TGGAGGGCAA GGTGATCCTG GTGGCTGTGC
ATGTGGCCTC CGGCTACATT GAGGCTGAGG TGATCCCTGC
TGAGACAGGC CAGGAGACTG CCTACTTCCT GCTGAAGCTG
GCTGGCAGGT GGCCTGTGAA GACCATCCAC ACTGACAATG
GCTCCAACTT CACTGGGGCC ACAGTGAGGG CTGCCTGCTG
GTGGGCTGGC ATCAAGCAGG AGTTTGGCAT CCCCTACAAC
CCCCAGTCCC AGGGGGTGGT GGAGTCCATG AACAAGGAGC
TGAAGAAGAT CATTGGGCAG GTGAGGGACC AGGCTGAGCA
CCTGAAGACA GCTGTGCAGA TGGCTGTGTT CATCCACAAC
TTCAAGAGGA AGGGGGGCAT CGGGGGCTAC TCCGCTGGGG
AGAGGATTGT GGACATCATT GCCACAGACA TCCAGACCAA
GGAGCTCCAG AAGCAGATCA CCAAGATCCA GAACTTCAGG
GTGTACTACA GGGACTCCAG GAACCCCCTG TGGAAGGGCC
CTGCCAAGCT GCTGTGGAAG GGGGAGGGGG CTGTGGTGAT
CCAGGACAAC TCTGACATCA AGGTGGTGCC CAGGAGGAAG
GCCAAGATCA TCAGGGACTA TGGCAAGCAG ATGGCTGGGG
ATGACTGTGT GGCCTCCAGG CAGGATGAGG ACTAAAGCCC
GGGCAGATCT.

The open reading frame of the wild type tPA-pol construct disclosed as SEQ ID NO:5 contains 875 amino acids, disclosed herein as SEQ ID NO:6, as follows:

(SEQ ID NO:6)

Met Asp Ala Met Lys Arg Gly Leu Cys Cys Val Leu
Leu Leu Cys Gly Ala Val Phe Val Ser Pro Ser Glu
Ile Ser Ala Pro Ile Ser Pro Ile Glu Thr Val Pro
Val Lys Leu Lys Pro Gly Met Asp Gly Pro Lys Val
Lys Gln Trp Pro Leu Thr Glu Glu Lys Ile Lys Ala
Leu Val Glu Ile Cys Thr Glu Met Glu Lys Glu Gly
Lys Ile Ser Lys Ile Gly Pro Glu Asn Pro Tyr Asn
Thr Pro Val Phe Ala Ile Lys Lys Lys Asp Ser Thr
Lys Trp Arg Lys Leu Val Asp Phe Arg Glu Leu Asn
Lys Arg Thr Gln Asp Phe Trp Glu Val Gln Leu Gly
Ile Pro His Pro Ala Gly Leu Lys Lys Lys Lys Ser
Val Thr Val Leu Asp Val Gly Asp Ala Tyr Phe Ser
Val Pro Leu Asp Glu Asp Phe Arg Lys Tyr Thr Ala
Phe Thr Ile Pro Ser Ile Asn Asn Glu Thr Pro Gly
Ile Arg Tyr Gln Tyr Asn Val Leu Pro Gln Gly Trp
Lys Gly Ser Pro Ala Ile Phe Gln Ser Ser Met Thr
Lys Ile Leu Glu Pro Phe Arg Lys Gln Asn Pro Asp
Ile Val Ile Tyr Gln Tyr Met Asp Asp Leu Tyr Val
Gly Ser Asp Leu Glu Ile Gly Gln His Arg Thr Lys
Ile Glu Glu Leu Arg Gln His Leu Leu Arg Trp Gly
Leu Thr Thr Pro Asp Lys Lys His Gln Lys Glu Pro
Pro Phe Leu Trp Met Gly Tyr Glu Leu His Pro Asp
Lys Trp Thr Val Gln Pro Ile Val Leu Pro Glu Lys
Asp Ser Trp Thr Val Asn Asp Ile Gln Lys Leu Val
Gly Lys Leu Asn Trp Ala Ser Gln Ile Tyr Pro Gly
Ile Lys Val Arg Gln Leu Cys Lys Leu Leu Arg Gly
Thr Lys Ala Leu Thr Glu Val Ile Pro Leu Thr Glu
Glu Ala Glu Leu Glu Leu Ala Glu Asn Arg Glu Ile
Leu Lys Glu Pro Val His Gly Val Tyr Tyr Asp Pro
Ser Lys Asp Leu Ile Ala Glu Ile Gln Lys Gln Gly
Gln Gly Gln Trp Thr Tyr Gln Ile Tyr Gln Glu Pro
Phe Lys Asn Leu Lys Thr Gly Lys Tyr Ala Arg Met
Arg Gly Ala His Thr Asn Asp Val Lys Gln Leu Thr
Glu Ala Val Gln Lys Ile Thr Thr Glu Ser Ile Val
Ile Trp Gly Lys Thr Pro Lys Phe Lys Leu Pro Ile
Gln Lys Glu Thr Trp Glu Thr Trp Trp Thr Glu Tyr
Trp Gln Ala Thr Trp Ile Pro Glu Trp Glu Phe Val
Asn Thr Pro Pro Leu Val Lys Leu Trp Tyr Gln Leu
Glu Lys Glu Pro Ile Val Gly Ala Glu Thr Phe Tyr
Val Asp Gly Ala Ala Asn Arg Glu Thr Lys Leu Gly
Lys Ala Gly Tyr Val Thr Asn Arg Gly Arg Gln Lys
Val Val Thr Leu Thr Asp Thr Thr Asn Gln Lys Thr
Glu Leu Gln Ala Ile Tyr Leu Ala Leu Gln Asp Ser
Gly Leu Glu Val Asn Ile Val Thr Asp Ser Gln Tyr
Ala Leu Gly Ile Ile Gln Ala Gln Pro Asp Gln Ser
Glu Ser Glu Leu Val Asn Gln Ile Ile Glu Gln Leu
Ile Lys Lys Glu Lys Val Tyr Leu Ala Trp Val Pro
Ala His Lys Gly Ile Gly Gly Asn Glu Gln Val Asp
Lys Leu Val Ser Ala Gly Ile Arg Lys Val Leu Phe
Leu Asp Gly Ile Asp Lys Ala Gln Asp Glu His Glu
Lys Tyr His Ser Asn Trp Arg Ala Met Ala Ser Asp
Phe Asn Leu Pro Pro Val Val Ala Lys Glu Ile Val
Ala Ser Cys Asp Lys Cys Gln Leu Lys Gly Glu Ala
Met His Gly Gln Val Asp Cys Ser Pro Gly Ile Trp
Gln Leu Asp Cys Thr His Leu Glu Gly Lys Val Ile
Leu Val Ala Val His Val Ala Ser Gly Tyr Ile Glu
Ala Glu Val Ile Pro Ala Glu Thr Gly Gln Glu Thr
Ala Tyr Phe Leu Leu Lys Leu Ala Gly Arg Trp Pro
Val Lys Thr Ile His Thr Asp Asn Gly Ser Asn Phe
Thr Gly Ala Thr Val Arg Ala Ala Cys Trp Trp Ala
Gly Ile Lys Gln Glu Phe Gly Ile Pro Tyr Asn Pro
Gln Ser Gln Gly Val Val Glu Ser Met Asn Lys Glu
Leu Lys Lys Ile Ile Gly Gln Val Arg Asp Gln Ala
Glu His Leu Lys Thr Ala Val Gln Met Ala Val Phe
Ile His Asn Phe Lys Arg Lys Gly Gly Ile Gly Gly
Tyr Ser Ala Gly Glu Arg Ile Val Asp Ile Ile Ala
Thr Asp Ile Gln Thr Lys Glu Leu Gln Lys Gln Ile
Thr Lys Ile Gln Asn Phe Arg Val Tyr Tyr Arg Asp
Ser Arg Asn Pro Leu Trp Lys Gly Pro Ala Lys Leu
Leu Trp Lys Gly Glu Gly Ala Val Val Ile Gln Asp
Asn Ser Asp Ile Lys Val Val Pro Arg Arg Lys Ala
Lys Ile Ile Arg Asp Tyr Gly Lys Gln Met Ala Gly
Asp Asp Cys Val Ala Ser Arg Gln Asp Glu Asp.

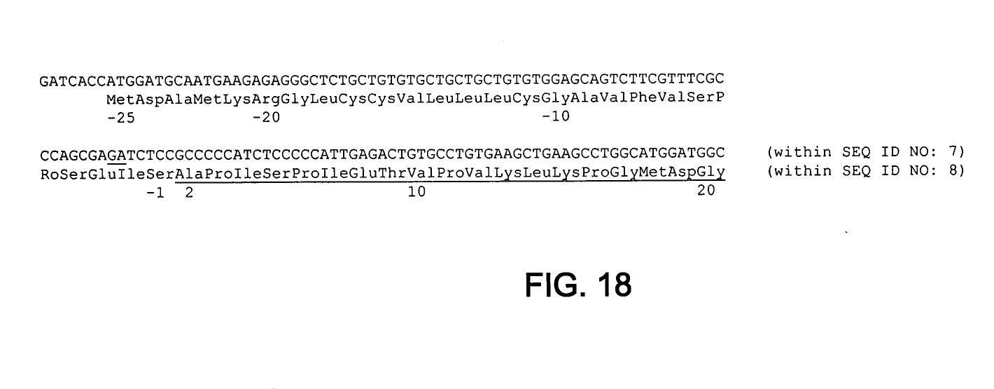

The present invention also relates to a codon optimized HIV-1 Pol mutant contained within a recombinant adenoviral vector such as IA-Pol (SEQ ID NO:4) which comprises a leader peptide at the amino terminal portion of the protein, which may effect cellular trafficking and hence, immunogenicity of the expressed protein within the host cell. Any such adenoviral-based HIV-1 DNA pol mutant disclosed in the above paragraphs is suitable for fusion downstream of a leader peptide, such as a leader peptide including but not limited to the human tPA leader sequence. Therefore, any such leader peptide-based HIV-1 pol mutant construct may include but is not limited to a mutated DNA molecule which effectively alters the catalytic activity of the RT, RNase and/or IN region of the expressed protein, resulting in at least substantially decreased enzymatic activity one or more of the RT, RNase H and/or IN functions of HIV-1 Pol. In a preferred embodiment of this portion of the invention, a leader peptide/HIV-1 DNA pol construct contains a mutation or mutations within the Pol coding region which effectively abolishes RT, RNase H and IN activity. An especially preferable HIV-1 DNA pol construct is a DNA molecule which contains at least one point mutation which alters the active site and catalytic activity within the RT, RNase H and IN domains of Pol, such that each activity is at least substantially abolished, and preferably totally abolished. Such a HIV-1 Pol mutant will most likely comprise at least one point mutation in or around each catalytic domain responsible for RT, RNase H and IN activity, respectfully. An especially preferred embodiment of this portion of the invention relates to a human tPA leader fused to the IA-Pol protein comprising the nine mutations shown in Table 1. The DNA molecule is disclosed herein as SEQ ID NO:7 and the expressed tPA-IA Pol protein comprises a fusion junction as shown in FIG. 18. The complete amino acid sequence of the expressed protein is set forth in SEQ ID NO:8. To this end, SEQ ID NO:7 discloses the nucleotide sequence which codes for a human tPA leader fused to the IA Pol protein comprising the nine mutations shown in Table 1 (herein, “tPA-opt-IApol”). The open reading frame begins with the initiating Met (nucleotides 8-10) and terminates with a “TAA” codon at nucleotides 2633-2635. The nucleotide sequence encoding tPA-IAPol is also disclosed as follows:

(SEQ ID NO:7)

GATCACCATG GATGCAATGA AGAGAGGGCT CTGCTGTGTG
CTGCTGCTGT GTGGAGCAGT CTTCGTTTCG CCCAGCGAGA
TCTCCGCCCC CATCTCCCCC ATTGAGACTG TGCCTGTGAA
GCTGAAGCCT GGCATGGATG GCCCCAAGGT GAAGCAGTGG
CCCCTGACTG AGGAGAAGAT CAAGGCCCTG GTGGAAATCT
GCACTGAGAT GGAGAAGGAG GGCAAAATCT CCAAGATTGG
CCCCGAGAAC CCCTACAACA CCCCTGTGTT TGCCATCAAG
AAGAAGGACT CCACCAAGTG GAGGAAGCTG GTGGACTTCA
GGGAGCTGAA CAAGAGGACC CAGGACTTCT GGGAGGTGCA
GCTGGGCATC CCCCACCCCG CTGGCCTGAA GAAGAAGAAG
TCTGTGACTG TGCTGGCTGT GGGGGATGCC TACTTCTCTG
TGCCCCTGGA TGAGGACTTC AGGAAGTACA CTGCCTTCAC
CATCCCCTCC ATCAACAATG AGACCCCTGG CATCAGGTAC
CAGTACAATG TGCTGCCCCA GGGCTGGAAG GGCTCCCCTG
CCATCTTCCA GTCCTCCATG ACCAAGATCC TGGAGCCCTT
CAGGAAGCAG AACCCTGACA TTGTGATCTA CCAGTACATG
GCTGCCCTGT ATGTGGGCTC TGACCTGGAG ATTGGGCAGC
ACAGGACCAA GATTGAGGAG CTGAGGCAGC ACCTGCTGAG
GTGGGGCCTG ACCACCCCTG ACAAGAAGCA CCAGAAGGAG
CCCCCCTTCC TGTGGATGGG CTATGAGCTG CACCCCGACA
AGTGGACTGT GCAGCCCATT GTGCTGCCTG AGAAGGACTC
CTGGACTGTG AATGACATCC AGAAGCTGGT GGGCAAGCTG
AACTGGGCCT CCCAAATCTA CCCTGGCATC AAGGTGAGGC
AGCTGTGCAA GCTGCTGAGG GGCACCAAGG CCCTGACTGA
GGTGATCCCC CTGACTGAGG AGGCTGAGCT GGAGCTGGCT
GAGAACAGGG AGATCCTGAA GGAGCCTGTG CATGGGGTGT
ACTATGACCC CTCCAAGGAC CTGATTGCTG AGATCCAGAA
GCAGGGCCAG GGCCAGTGGA CCTACCAAAT CTACCAGGAG
CCCTTCAAGA ACCTGAAGAC TGGCAAGTAT GCCAGGATGA
GGGGGGCCCA CACCAATGAT GTGAAGCAGC TGACTGAGGC
TGTGCAGAAG ATCACCACTG AGTCCATTGT GATCTGGGGC
AAGACCCCCA AGTTCAAGCT GCCCATCCAG AAGGAGACCT
GGGAGACCTG GTGGACTGAG TACTGGCAGG CCACCTGGAT
CCCTGAGTGG GAGTTTGTGA ACACCCCCCC CCTGGTGAAG
CTGTGGTACC AGCTGGAGAA GGAGCCCATT GTGGGGGCTG
AGACCTTCTA TGTGGCTGGG GCTGCCAACA GGGAGACCAA
GCTGGGCAAG GCTGGCTATG TGACCAACAG GGGCAGGCAG
AAGGTGGTGA CCCTGACTGA CACCACCAAC CAGAAGACTG
CCCTCCAGGC CATCTACCTG GCCCTCCAGG ACTCTGGCCT
GGAGGTGAAC ATTGTGACTG CCTCCCAGTA TGCCCTGGGC
ATCATCCAGG CCCAGCCTGA TCAGTCTGAG TCTGAGCTGG
TGAACCAGAT CATTGAGCAG CTGATCAAGA AGGAGAAGGT
GTACCTGGCC TGGGTGCCTG CCCACAAGGG CATTGGGGGC
AATGAGCAGG TGGACAAGCT GGTGTCTGCT GGCATCAGGA
AGGTGCTGTT CCTGGATGGC ATTGACAAGG CCCAGGATGA
GCATGAGAAG TACCACTCCA ACTGGAGGGC TATGGCCTCT
GACTTCAACC TGCCCCCTGT GGTGGCTAAG GAGATTGTGG
CCTCCTGTGA CAAGTGCCAG CTGAAGGGGG AGGCCATGCA
TGGGCAGGTG GACTGCTCCC CTGGCATCTG GCAGCTGGCC
TGCACCCACC TGGAGGGCAA GGTGATCCTG GTGGCTGTGC
ATGTGGCCTC CGGCTACATT GAGGCTGAGG TGATCCCTGC
TGAGACAGGC CAGGAGACTG CCTACTTCCT GCTGAAGCTG
GCTGGCAGGT GGCCTGTGAA GACCATCCAC ACTGCCAATG
GCTCCAACTT CACTGGGGCC ACAGTGAGGG CTGCCTGCTG
GTGGGCTGGC ATCAAGCAGG AGTTTGGCAT CCCCTACAAC
CCCCAGTCCC AGGGGGTGGT GGCCTCCATG AACAAGGAGC
TGAAGAAGAT CATTGGGCAG GTGAGGGACC AGGCTGAGCA
CCTGAAGACA GCTGTGCAGA TGGCTGTGTT CATCCACAAC
TTCAAGAGGA AGGGGGGCAT CGGGGGCTAC TCCGCTGGGG
AGAGGATTGT GGACATCATT GCCACAGACA TCCAGACCAA
GGAGCTCCAG AAGCAGATCA CCAAGATCCA GAACTTCAGG
GTGTACTACA GGCACTCCAG GAACCCCCTG TGGAAGGGCC
CTGCCAAGCT GCTGTGGAAG GGGGAGGGGG CTGTGGTGAT
CCAGGACAAC TCTGACATCA AGGTGGTGCC CAGGAGGAAG
GCCAAGATCA TCAGGGACTA TGGCAAGCAG ATGGCTGGGG
ATGACTGTGT GGCCTCCAGG CAGGATGAGG ACTAAAGCCC
GGGCAGATCT.

The open reading frame of the tPA-IA-pol construct disclosed as SEQ ID NO:7 contains 875 amino acids, disclosed herein as tPA-IA-Pol and SEQ ID NO:8, as follows:

(SEQ ID NO:7)

Met Asp Ala Met Lys Arg Gly Leu Cys Cys Val Leu
Leu Leu Cys Gly Ala Val Phe Val Ser Pro Ser Glu
Ile Ser Ala Pro Ile Ser Pro Ile Glu Thr Val Pro
Val Lys Leu Lys Pro Gly Met Asp Gly Pro Lys Val
Lys Gln Trp Pro Leu Thr Glu Glu Lys Ile Lys Ala
Leu Val Glu Ile Cys Thr Glu Met Glu Lys Glu Gly
Lys Ile Ser Lys Ile Gly Pro Glu Asn Pro Tyr Asn
Thr Pro Val Phe Ala Ile Lys Lys Lys Asp Ser Thr
Lys Trp Arg Lys Leu Val Asp Phe Arg Glu Leu Asn
Lys Arg Thr Gln Asp Phe Trp Glu Val Gln Leu Gly
Ile Pro His Pro Ala Gly Leu Lys Lys Lys Lys Ser
Val Thr Val Leu Ala Val Gly Asp Ala Tyr Phe Ser
Val Pro Leu Asp Glu Asp Phe Arg Lys Tyr Thr Ala
Phe Thr Ile Pro Ser Ile Asn Asn Glu Thr Pro Gly
Ile Arg Tyr Gln Tyr Asn Val Leu Pro Gln Gly Trp
Lys Gly Ser Pro Ala Ile Phe Gln Ser Ser Met Thr
Lys Ile Leu Glu Pro Phe Arg Lys Gln Asn Pro Asp
Ile Val Ile Tyr Gln Tyr Met Ala Ala Leu Tyr Val
Gly Ser Asp Leu Glu Ile Gly Gln His Arg Thr Lys
Ile Glu Glu Leu Arg Gln His Leu Leu Arg Trp Gly
Leu Thr Thr Pro Asp Lys Lys His Gln Lys Glu Pro
Pro Phe Leu Trp Met Gly Tyr Glu Leu His Pro Asp
Lys Trp Thr Val Gln Pro Ile Val Leu Pro Glu Lys
Asp Ser Trp Thr Val Asn Asp Ile Gln Lys Leu Val
Gly Lys Leu Asn Trp Ala Ser Gln Ile Tyr Pro Gly
Ile Lys Val Arg Gln Leu Cys Lys Leu Leu Arg Gly
Thr Lys Ala Leu Thr Glu Val Ile Pro Leu Thr Glu
Glu Ala Glu Leu Glu Leu Ala Glu Asn Arg Glu Ile
Leu Lys Glu Pro Val His Gly Val Tyr Tyr Asp Pro
Ser Lys Asp Leu Ile Ala Glu Ile Gln Lys Gln Gly
Gln Gly Gln Trp Thr Tyr Gln Ile Tyr Gln Glu Pro
Phe Lys Asn Leu Lys Thr Gly Lys Tyr Ala Arg Met
Arg Gly Ala His Thr Asn Asp Val Lys Gln Leu Thr
Glu Ala Val Gln Lys Ile Thr Thr Glu Ser Ile Val
Ile Trp Gly Lys Thr Pro Lys Phe Lys Leu Pro Ile
Gln Lys Glu Thr Trp Glu Thr Trp Trp Thr Glu Tyr
Trp Gln Ala Thr Trp Ile Pro Glu Trp Glu Phe Val
Asn Thr Pro Pro Leu Val Lys Leu Trp Tyr Gln Leu
Glu Lys Glu Pro Ile Val Gly Ala Glu Thr Phe Tyr
Val Ala Gly Ala Ala Asn Arg Glu Thr Lys Leu Gly
Lys Ala Gly Tyr Val Thr Asn Arg Gly Arg Gln Lys
Val Val Thr Leu Thr Asp Thr Thr Asn Gln Lys Thr
Ala Leu Gln Ala Ile Tyr Leu Ala Leu Gln Asp Ser
Gly Leu Glu Val Asn Ile Val Thr Ala Ser Gln Tyr
Ala Leu Gly Ile Ile Gln Ala Gln Pro Asp Gln Ser
Glu Ser Glu Leu Val Asn Gln Ile Ile Glu Gln Leu
Ile Lys Lys Glu Lys Val Tyr Leu Ala Trp Val Pro
Ala His Lys Gly Ile Gly Gly Asn Glu Gln Val Asp
Lys Leu Val Ser Ala Gly Ile Arg Lys Val Leu Phe
Leu Asp Gly Ile Asp Lys Ala Gln Asp Glu His Glu
Lys Tyr His Ser Asn Trp Arg Ala Met Ala Ser Asp
Phe Asn Leu Pro Pro Val Val Ala Lys Glu Ile Val
Ala Ser Cys Asp Lys Cys Gln Leu Lys Gly Glu Ala
Met His Gly Gln Val Asp Cys Ser Pro Gly Ile Trp
Gln Leu Ala Cys Thr His Leu Glu Gly Lys Val Ile
Leu Val Ala Val His Val Ala Ser Gly Tyr Ile Glu
Ala Glu Val Ile Pro Ala Glu Thr Gly Gln Glu Thr
Ala Tyr Phe Leu Leu Lys Leu Ala Gly Arg Trp Pro
Val Lys Thr Ile His Thr Ala Asn Gly Ser Asn Phe
Thr Gly Ala Thr Val Arg Ala Ala Cys Trp Trp Ala
Gly Ile Lys Gln Glu Phe Gly Ile Pro Tyr Asn Pro
Gln Ser Gln Gly Val Val Ala Ser Met Asn Lys Glu
Leu Lys Lys Ile Ile Gly Gln Val Arg Asp Gln Ala
Glu His Leu Lys Thr Ala Val Gln Met Ala Val Phe
Ile His Asn Phe Lys Arg Lys Gly Gly Ile Gly Gly
Tyr Ser Ala Gly Glu Arg Ile Val Asp Ile Ile Ala
Thr Asp Ile Gln Thr Lys Glu Leu Gln Lys Gln Ile
Thr Lys Ile Gln Asn Phe Arg Val Tyr Tyr Arg Asp
Ser Arg Asn Pro Leu Trp Lys Gly Pro Ala Lys Leu
Leu Trp Lys Gly Glu Gly Ala Val Val Ile Gln Asp
Asn Ser Asp Ile Lys Val Val Pro Arg Arg Lys Ala
Lys Ile Ile Arg Asp Tyr Gly Lys Gln Met Ala Gly
Asp Asp Cys Val Ala Ser Arg Gln Asp Glu Asp.

### EXAMPLE 18

### Codon Optimized HIV-1 Nef and Codon Optimized HIV-1 Nef Modifications

Codon optimized version of HIV-1 Nef and HIV-1 Nef modifications are essentially as described in U.S. application Ser. No. 09/738,782, filed Dec. 15, 2000 and PCT International Application PCT/US00/34162, also filed Dec. 15, 2000, both documents which are hereby incorporated by reference. As disclosed within the above-mentioned documents, particular embodiments of codon optimized Nef and Nef modifications relate to a DNA molecule encoding HIV-1 Nef from the HIV-1 jfrl isolate wherein the codons are optimized for expression in a mammalian system such as a human. The DNA molecule which encodes this protein is disclosed herein as SEQ ID NO:9, while the expressed open reading frame is disclosed herein as SEQ ID NO:10. Another embodiment of Nef-based coding regions for use in the adenoviral vectors of the present invention comprise a codon optimized DNA molecule encoding a protein containing the human plasminogen activator (tpa) leader peptide fused with the NH₂-terminus of the HIV-1 Nef polypeptide. The DNA molecule which encodes this protein is disclosed herein as SEQ ID NO:11, while the expressed open reading frame is disclosed herein as SEQ ID NO:12. Another modified Nef optimized coding region relates to a DNA molecule encoding optimized HIV-1 Nef wherein the open reading frame codes for modifications at the amino terminal myristylation site (Gly-2 to Ala-2) and substitution of the Leu-174-Leu-175 dileucine motif to Ala-174-Ala-175, herein described as opt nef (G2A, LLAA). The DNA molecule which encodes this protein is disclosed herein as SEQ ID NO:13, while the expressed open reading frame is disclosed herein as SEQ ID NO:14. An additional embodiment relates to a DNA molecule encoding optimized HIV-1 Nef wherein the amino terminal myristylation site and dileucine motif have been deleted, as well as comprising a tPA leader peptide. This DNA molecule, opt tpanef (LLAA), comprises an open reading frame which encodes a Nef protein containing a tPA leader sequence fused to amino acid residue 6-216 of HIV-1 Nef (jfrl), wherein Leu-174 and Leu-175 are substituted with Ala-174 and Ala-175, herein referred to as opt tpanef (LLAA) is disclosed herein as SEQ ID NO:15, while the expressed open reading frame is disclosed herein as SEQ ID NO:16.

As disclosed in the above-identified documents (U.S. application Ser. No. 09/738,782 and PCT International Application PCT/US00/34162) and reiterated herein, the following nef-based nucleotide and amino acid sequences which comprise the respective open reading frame are as follows:

1. The nucleotide sequence of the codon optimized version of HIV-1 jrfl nef gene is disclosed herein as SEQ ID NO:9, as shown herein:

(SEQ ID NO:9)

GATCTGCCAC CATGGGCGGC AAGTGGTCCA AGAGGTCCGT
GCCCGGCTGG TCCACCGTGA GGGAGAGGAT GAGGAGGGCC
GAGCCCGCCG CCGACAGGGT GAGGAGGACC GAGCCCGCCG
CCGTGGGCGT GGGCGCCGTG TCCAGGGACC TGGAGAAGCA
CGGCGCCATC ACCTCCTCCA ACACCGCCGC CACCAACGCC
GACTGCGCCT GGCTGGAGGC CCAGGAGGAC GAGGAGGTGG
GCTTCCCCGT GAGGCCCCAG GTGCCCCTGA GGCCCATGAC
CTACAAGGGC GCCGTGGACC TGTCCCACTT CCTGAAGGAG
AAGGGCGGCC TGGAGGGCCT GATCCACTCC CAGAAGAGGC
AGGACATCCT GGACCTGTGG GTGTACCACA CCCAGGGCTA
CTTCCCCGAC TGGCAGAACT ACACCCCCGG CCCCGGCATC
AGGTTCCCCC TGACCTTCGG CTGGTGCTTC AAGCTGGTGC
CCGTGGAGCC CGAGAAGGTG GAGGAGGCCA ACGAGGGCGA
GAACAACTGC CTGCTGCACC CCATGTCCCA GCACGGCATC
GAGGACCCCG AGAAGGAGGT GCTGGAGTGG AGGTTCGACT
CCAAGCTGGC CTTCCACCAC GTGGCCAGGG AGCTGCACCC
CGAGTACTAC AAGGACTGCT AAAGCCCGGG C.

Preferred codon usage is as follows: Met (ATG), Gly (GGC), Lys (AAG), Trp (TGG), Ser (TCC), Arg (AGG), Val (GTG), Pro (CCC), Thr (ACC), Glu (GAG); Leu (CTG), His (CAC), le (ATC), Asn (AAC), Cys (TGC), Ala (GCC), Gln (CAG), Phe (TTC) and Tyr (TAC). For an additional discussion relating to mammalian (human) codon optimization, see WO 97/31115 (PCT/US97/02294), which is hereby incorporated by reference. See also FIGS. 19A-1 to 19A-2 for a comparison of wild type vs. codon optimized nucleotides comprising the open reading frame of HIV-Nef.

The open reading frame for SEQ ID NO:9 above comprises an initiating methionine residue at nucleotides 12-14 and a “TAA” stop codon from nucleotides 660-662. The open reading frame of SEQ ID NO:9 provides for a 216 amino acid HIV-1 Nef protein expressed through utilization of a codon optimized DNA vaccine vector. The 216 amino acid HIV-1 Nef (jfrl) protein is disclosed herein as SEQ ID NO:10, and as follows:

(SEQ ID NO:10)

Met Gly Gly Lys Trp Ser Lys Arg Ser Val Pro Gly
Trp Ser Thr Val Arg Glu Arg Met Arg Arg Ala Glu
Pro Ala Ala Asp Arg Val Arg Arg Thr Glu Pro Ala
Ala Val Gly Val Gly Ala Val Ser Arg Asp Leu Glu
Lys His Gly Ala Ile Thr Ser Ser Asn Thr Ala Ala
Thr Asn Ala Asp Cys Ala Trp Leu Glu Ala Gln Glu
Asp Glu Glu Val Gly Phe Pro Val Arg Pro Gln Val
Pro Leu Arg Pro Met Thr Tyr Lys Gly Ala Val Asp
Leu Ser His Phe Leu Lys Glu Lys Gly Gly Leu Glu
Gly Leu Ile His Ser Gln Lys Arg Gln Asp Ile Leu
Asp Leu Trp Val Tyr His Thr Gln Gly Tyr Phe Pro
Asp Trp Gln Asn Tyr Thr Pro Gly Pro Gly Ile Arg
Phe Pro Leu Thr Phe Gly Trp Cys Phe Lys Leu Val
Pro Val Glu Pro Glu Lys Val Glu Glu Ala Asn Glu
Gly Glu Asn Asn Cys Leu Leu His Pro Met Ser Gln
His Gly Ile Glu Asp Pro Glu Lys Glu Val Leu Glu
Trp Arg Phe Asp Ser Lys Leu Ala Phe His His Val
Ala Arg Glu Leu His Pro Glu Tyr Tyr Lys Asp Cys.

HIV-1 Nef is a 216 amino acid cytosolic protein which associates with the inner surface of the host cell plasma membrane through myristylation of Gly-2 (Franchini et al., 1986, Virology 155: 593-599). While not all possible Nef functions have been elucidated, it has become clear that correct trafficking of Nef to the inner plasma membrane promotes viral replication by altering the host intracellular environment to facilitate the early phase of the HIV-1 life cycle and by increasing the infectivity of progeny viral particles. In one aspect of the invention regarding codon-optimized, protein-modified polypeptides, the nef-encoding region of the adenovirus vector of the present invention is modified to contain a nucleotide sequence which encodes a heterologous leader peptide such that the amino terminal region of the expressed protein will contain the leader peptide. The diversity of function that typifies eukaryotic cells depends upon the structural differentiation of their membrane boundaries. To generate and maintain these structures, proteins must be transported from their site of synthesis in the endoplasmic reticulum to predetermined destinations throughout the cell. This requires that the trafficking proteins display sorting signals that are recognized by the molecular machinery responsible for route selection located at the access points to the main trafficking pathways. Sorting decisions for most proteins need to be made only once as they traverse their biosynthetic pathways since their final destination, the cellular location at which they perform their function, becomes their permanent residence. Maintenance of intracellular integrity depends in part on the selective sorting and accurate transport of proteins to their correct destinations. Defined sequence motifs exist in proteins which can act as ‘address labels’. A number of sorting signals have been found associated with the cytoplasmic domains of membrane proteins. An effective induction of CTL responses often required sustained, high level endogenous expression of an antigen. As membrane-association via myristylation is an essential requirement for most of Nef s function, mutants lacking myristylation, by glycine-to-alanine change, change of the dileucine motif and/or by substitution with a tpa leader sequence as described herein, will be functionally defective, and therefore will have improved safety profile compared to wild-type Nef for use as an HIV-1 vaccine component.

In another embodiment of this portion of the invention, either the DNA vector or the HIV-I nef nucleotide sequence is modified to include the human tissue-specific plasminogen activator (tPA) leader. As shown in FIG. 16A-B, a DNA vector may be modified by known recombinant DNA methodology to contain a leader signal peptide of interest, such that downstream cloning of the modified HIV-1 protein of interest results in a nucleotide sequence which encodes a modified HIV-1 tpA/Nef protein. In the alternative, as noted above, insertion of a nucleotide sequence which encodes a leader peptide may be inserted into a DNA vector housing the open reading frame for the Nef protein of interest. Regardless of the cloning strategy, the end result is a polynucleotide vaccine which comprises vector components for effective gene expression in conjunction with nucleotide sequences which encode a modified HIV-1 Nef protein of interest, including but not limited to a HIV-1 Nef protein which contains a leader peptide. The amino acid sequence of the human tPA leader utilized herein is as follows:

(SEQ ID NO:17)

MDAMKRGLCCVLLLCGAVFVSPSEISS.

It has been shown that myristylation of Gly-2 in conjunction with a dileucine motif in the carboxy region of the protein is essential for Nef-induced down regulation of CD4 (Aiken et al., 1994, Cell 76: 853-864) via endocytosis. It has also been shown that Nef expression promotes down regulation of MHCI (Schwartz et al., 1996, Nature Medicine 2(3): 338-342) via endocytosis. The present invention relates in part to DNA vaccines which encode modified Nef proteins altered in trafficking and/or functional properties. The modifications introduced into the adenoviral vector HIV vaccines of the present invention include but are not limited to additions, deletions or substitutions to the nef open reading frame which results in the expression of a modified Nef protein which includes an amino terminal leader peptide, modification or deletion of the amino terminal myristylation site, and modification or deletion of the dileucine motif within the Nef protein and which alter function within the infected host cell. Therefore, a central theme of the DNA molecules and recombinant adenoviral HIV vaccines of the present invention is (1) host administration and intracellular delivery of a codon optimized nef-based adenoviral HIV vaccine; (2) expression of a modified Nef protein which is immunogenic in terms of eliciting both CTL and Th responses; and, (3) inhibiting or at least altering known early viral functions of Nef which have been shown to promote HIV-1 replication and load within an infected host. Therefore, the nef coding region may be altered, resulting in a DNA vaccine which expresses a modified Nef protein wherein the amino terminal Gly-2 myristylation residue is either deleted or modified to express alternate amino acid residues. Also, the nef coding region may be altered so as to result in a DNA vaccine which expresses a modified Nef protein wherein the dileucine motif is either deleted or modified to express alternate amino acid residues. In addition, the adenoviral vector HIV vaccines of the present invention also relate to an isolated DNA molecule, regardless of codon usage, which expresses a wild type or modified Nef protein as described herein, including but not limited to modified Nef proteins which comprise a deletion or substitution of Gly 2, a deletion or substitution of Leu 174 and Leu 175 and/or inclusion of a leader sequence.

Therefore, specific Nef-based constructs further include the following, as exemplification's and not limitations. For example, the present invention relates to an adenoviral vector vaccine which encodes modified forms of HIV-1, an open reading frame which encodes a Nef protein which comprises a tpA leader sequence fused to amino acid residue 6-216 of HIV-1 Nef (jfrl) is referred to herein as opt tpanef. The nucleotide sequence comprising the open reading frame of opt tpanef is disclosed herein as SEQ ID NO:11, as shown below:

(SEQ ID NO:11)

CATGGATGCA ATGAAGAGAG GGCTCTGCTG TGTGCTGCTG
CTGTGTGGAG CAGTCTTCGT TTCGCCCAGC GAGATCTCCT
CCAAGAGGTC CGTGCCCGGC TGGTCCACCG TGAGGGAGAG
GATGAGGAGG GCCGAGCCCG CCGCCGACAG GGTGAGGAGG
ACCGAGCCCG CCGCCGTGGG CGTGGGCGCC GTGTCCAGGG
ACCTGGAGAA GCACGGCGCC ATCACCTCCT CCAACACCGC
CGCCACCAAC GCCGACTGCG CCTGGCTGGA GGCCCAGGAG
GACGAGGAGG TCGGCTTCCC CGTGAGGCCC CAGGTGCCCC
TGAGGCCCAT GACCTACAAG GGCGCCGTGG ACCTGTCCCA
CTTCCTGAAG GAGAAGGGCG GCCTGGAGGG CCTGATCCAC
TCCCAGAAGA GGCAGGACAT CCTGGACCTG TGGGTGTACC
ACACCCAGGG CTACTTCCCC GACTGGCAGA ACTACACCCC
CGGCCCCGGC ATCAGGTTCC CCCTGACCTT CGGCTGGTGC
TTCAAGCTGG TGCCCGTGGA GCCCGAGAAG GTGGAGGAGG
CCAACGAGGG CGAGAACAAC TGCCTGCTGC ACCCCATGTC
CCAGCACGGC ATCGAGGACC CCGAGAAGGA GGTGCTGGAG
TGGAGGTTCG ACTCCAAGCT GGCCTTCCAC CACGTGGCCA
GGGAGCTGCA CCCCGAGTAC TACAAGGACT GCTAAAGCC.

The open reading frame for SEQ ID NO:11 comprises an initiating methionine residue at nucleotides 2-4 and a “TAA” stop codon from nucleotides 713-715. The open reading frame of SEQ ID NO: 11 provides for a 237 amino acid HIV-1 Nef protein which comprises a tPA leader sequence fused to amino acids 6-216 of HIV-1 Nef, including the dileucine motif at amino acid residues 174 and 175. This 237 amino acid tPA/Nef (jfrl) fusion protein is disclosed herein as SEQ ID NO:12, and is shown as follows:

(SEQ ID NO:12)

Met Asp Ala Met Lys Arg Gly Leu Cys Cys Val Leu
Leu Leu Cys Gly Ala Val Phe Val Ser Pro Ser Glu
Ile Ser Ser Lys Arg Ser Val Pro Gly Trp Ser Thr
Val Arg Glu Arg Met Arg Arg Ala Glu Pro Ala Ala
Asp Arg Val Arg Arg Thr Glu Pro Ala Ala Val Gly
Val Gly Ala Val Ser Arg Asp Leu Glu Lys His Gly
Ala Ile Thr Ser Ser Asn Thr Ala Ala Thr Asn Ala
Asp Cys Ala Trp Leu Glu Ala Gln Glu Asp Glu Glu
Val Gly Phe Pro Val Arg Pro Gln Val Pro Leu Arg
Pro Met Thr Tyr Lys Gly Ala Val Asp Leu Ser His
Phe Leu Lys Glu Lys Gly Gly Leu Glu Gly Leu Ile
His Ser Gln Lys Arg Gln Asp Ile Leu Asp Leu Trp
Val Tyr His Thr Gln Gly Tyr Phe Pro Asp Trp Gln
Asn Tyr Thr Pro Gly Pro Gly Ile Arg Phe Pro Leu
Thr Phe Gly Trp Cys Phe Lys Leu Val Pro Val Glu
Pro Glu Lys Val Glu Glu Ala Asn Glu Gly Glu Asn
Asn Cys Leu Leu His Pro Met Ser Gln His Gly Ile
Glu Asp Pro Glu Lys Glu Val Leu Glu Trp Arg Phe
Asp Ser Lys Leu Ala Phe His His Val Ala Arg Glu
Leu His Pro Glu Tyr Tyr Lys Asp Cys.

Therefore, this exemplified Nef protein, Opt tPA-Nef, contains both a tPA leader sequence as well as deleting the myristylation site of Gly-2A DNA molecule encoding HIV-1 Nef from the HIV-1 jfrl isolate wherein the codons are optimized for expression in a mammalian system such as a human.

In another specific embodiment of the present invention, a DNA molecule is disclosed which encodes optimized HIV-1 Nef wherein the open reading frame of a recombinant adenoviral HIV vaccine encodes for modifications at the amino terminal myristylation site (Gly-2 to Ala-2) and substitution of the Leu-174-Leu-175 dileucine motif to Ala-174-Ala-175. This open reading frame is herein described as opt nef (G2A,LLAA) and is disclosed as SEQ ID NO: 13, which comprises an initiating methionine residue at nucleotides 12-14 and a “TAA” stop codon from nucleotides 660-662. The nucleotide sequence of this codon optimized version of HIV-1 jrfl nef gene with the above mentioned modifications is disclosed herein as SEQ ID NO:13, as follows:

(SEQ ID NO:13)

GATCTGCCAC CATGGCCGGC AAGTGGTCCA AGAGGTCCGT
GCCCGGCTGG TCCACCGTGA GGGAGAGGAT GAGGAGGGCC
GAGCCCGCCG CCGACAGGGT GAGGAGGACC GAGCCCGCCG
CCGTGGGCGT GGGCGCCGTG TCCAGGGACC TGGAGAAGCA
CGGCGCCATC ACCTCCTCCA ACACCGCCGC CACCAACGCC
GACTGCGCCT GGCTGGAGGC CCAGGAGGAC GAGGAGGTGG
GCTTCCCCGT GAGGCCCCAG GTGCCCCTGA GGCCCATGAC
CTACAAGGGC GCCGTGGACC TGTCCCACTT CCTGAAGGAG
AAGGGCGGCC TGGAGGGCCT GATCCACTCC CAGAAGAGGC
AGGACATCCT GGACCTGTGG GTGTACCACA CCCAGGGCTA
CTTCCCCGAC TGGCAGAACT ACACCCCCGG CCCCGGCATC
AGGTTCCCCC TGACCTTCGG CTGGTGCTTC AAGCTGGTGC
CCGTGGAGCC CGAGAAGGTG GAGGAGGCCA ACGAGGGCGA
GAACAACTGC GCCGCCCACC CCATGTCCCA GCACGGCATC
GAGGACCCCG AGAAGGAGGT GCTGGAGTGG AGGTTCGACT
CCAAGCTGGC CTTCCACCAC GTGGCCAGGG AGCTGCACCC
CGAGTACTAC AAGGACTGCT AAAGCCCGGG C.

The open reading frame of SEQ ID NO:13 encodes Nef (G2A,LLAA), disclosed herein as SEQ ID NO:14, as follows:

(SEQ ID NO:14)

Met Ala Gly Lys Trp Ser Lys Arg Ser Val Pro Gly
Trp Ser Thr Val Arg Glu Arg Met Arg Arg Ala Glu
Pro Ala Ala Asp Arg Val Arg Arg Thr Glu Pro Ala
Ala Val Gly Val Gly Ala Val Ser Arg Asp Leu Glu
Lys His Gly Ala Ile Thr Ser Ser Asn Thr Ala Ala
Thr Asn Ala Asp Cys Ala Trp Leu Glu Ala Gln Glu
Asp Glu Glu Val Gly Phe Pro Val Arg Pro Gln Val
Pro Leu Arg Pro Met Thr Tyr Lys Gly Ala Val Asp
Leu Ser His Phe Leu Lys Glu Lys Gly Gly Leu Glu
Gly Leu Ile His Ser Gln Lys Arg Gln Asp Ile Leu
Asp Leu Trp Val Tyr His Thr Gln Gly Tyr Phe Pro
Asp Trp Gln Asn Tyr Thr Pro Gly Pro Gly Ile Arg
Phe Pro Leu Thr Phe Gly Trp Cys Phe Lys Leu Val
Pro Val Glu Pro Glu Lys Val Glu Glu Ala Asn Glu
Gly Glu Asn Asn Cys Ala Ala His Pro Met Ser Gln
His Gly Ile Glu Asp Pro Glu Lys Glu Val Leu Glu
Trp Arg Phe Asp Ser Lys Leu Ala Phe His His Val
Ala Arg Glu Leu His Pro Glu Tyr Tyr Lys Asp Cys
Ser.

An additional embodiment of the present invention relates to another DNA molecule encoding optimized HIV-1 Nef wherein the amino terminal myristylation site and dileucine motif have been deleted, as well as comprising a tPA leader peptide. This DNA molecule, opt tpanef (LLAA) comprises an open reading frame which encodes a Nef protein containing a tPA leader sequence fused to amino acid residue 6-216 of HIV-1 Nef (jfrl), wherein Leu-174 and Leu-175 are substituted with Ala-174 and Ala-175 (Ala-195 and Ala-196 in this tPA-based fusion protein). The nucleotide sequence comprising the open reading frame of opt tpanef (LLAA) is disclosed herein as SEQ ID NO:15, as shown below:

(SEQ ID NO:15)

CATGGATGCA ATGAAGAGAG GGCTCTGCTG TGTGCTGCTG
CTGTGTGGAG CAGTCTTCGT TTCGCCCAGC GAGATCTCCT
CCAAGAGGTC CGTGCCCGGC TGGTCCACCG TGAGGGAGAG
GATGAGGAGG GCCGAGCCCG CCGCCGACAG GGTGAGGAGG
ACCGAGCCCG CCGCCGTGGG CGTGGGCGCC GTGTCCAGGG
ACCTGGAGAA GCACGGCGCC ATCACCTCCT CCAACACCGC
CGCCACCAAC GCCGACTGCG CCTGGCTGGA GGCCCAGGAG
GACGAGGAGG TGGGCTTCCC CGTGAGGCCC CAGGTGCCCC
TGAGGCCCAT GACCTACAAG GGCGCCGTGG ACCTGTCCCA
CTTCCTGAAG GAGAAGGGCG GCCTGGAGGG CCTGATCCAC
TCCCAGAAGA GGCAGGACAT CCTGGACCTG TGGGTGTACC
ACACCCAGGG CTACTTCCCC GACTGGCAGA ACTACACCCC
CGGCCCCGGC ATCAGGTTCC CCCTGACCTT CGGCTGGTGC
TTCAAGCTGG TGCCCGTGGA GCCCGAGAAG GTGGAGGAGG
CCAACGAGGG CGAGAACAAC TGCGCCGCCC ACCCCATGTC
CCAGCACGGC ATCGAGGACC CCGAGAAGGA GGTGCTGGAG
TGGAGGTTCG ACTCCAAGCT GGCCTTCCAC CACGTGGCCA
GGGAGCTGCA CCCCGAGTAC TACAAGGACT GCTAAAGCCC.

The open reading frame of SEQ ID NO: 15 encoding tPA-Nef (LLAA), disclosed herein as SEQ ID NO:16, is as follows:

(SEQ ID NO:16)

Met Asp Ala Met Lys Arg Gly Leu Cys Cys Val Leu
Leu Leu Cys Gly Ala Val Phe Val Ser Pro Ser Glu
Ile Ser Ser Lys Arg Ser Val Pro Gly Trp Ser Thr
Val Arg Glu Arg Met Arg Arg Ala Glu Pro Ala Ala
Asp Arg Val Arg Arg Thr Glu Pro Ala Ala Val Gly
Val Gly Ala Val Ser Arg Asp Leu Glu Lys His Gly
Ala Ile Thr Ser Ser Asn Thr Ala Ala Thr Asn Ala
Asp Cys Ala Trp Leu Glu Ala Gln Glu Asp Glu Glu
Val Gly Phe Pro Val Arg Pro Gln Val Pro Leu Arg
Pro Met Thr Tyr Lys Gly Ala Val Asp Leu Ser His
Phe Leu Lys Glu Lys Gly Gly Leu Glu Gly Leu Ile
His Ser Gln Lys Arg Gln Asp Ile Leu Asp Leu Trp
Val Tyr His Thr Gln Gly Tyr Phe Pro Asp Trp Gln
Asn Tyr Thr Pro Gly Pro Gly Ile Arg Phe Pro Leu
Thr Phe Gly Trp Cys Phe Lys Leu Val Pro Val Glu
Pro Glu Lys Val Glu Glu Ala Asn Glu Gly Glu Asn
Asn Cys Ala Ala His Pro Met Ser Gln His Gly Ile
Glu Asp Pro Glu Lys Glu Val Leu Glu Trp Arg Phe
Asp Ser Lys Leu Ala Phe His His Val Ala Arg Glu
Leu His Pro Glu Tyr Tyr Lys Asp Cys.

An adenoviral vector of the present invention may comprise a DNA sequence, regardless of codon usage, which expresses a wild type or modified Nef protein as described herein, including but not limited to modified Nef proteins which comprise a deletion or substitution of Gly 2, a deletion of substitution of Leu 174 and Leu 175 and/or inclusion of a leader sequence. Therefore, partial or fully codon optimized DNA vaccine expression vector constructs are preferred since such constructs should result in increased host expression. However, it is within the scope of the present invention to utilize “non-codon optimized” versions of the constructs disclosed herein, especially modified versions of HIV Nef which are shown to promote a substantial cellular immune response subsequent to host administration.

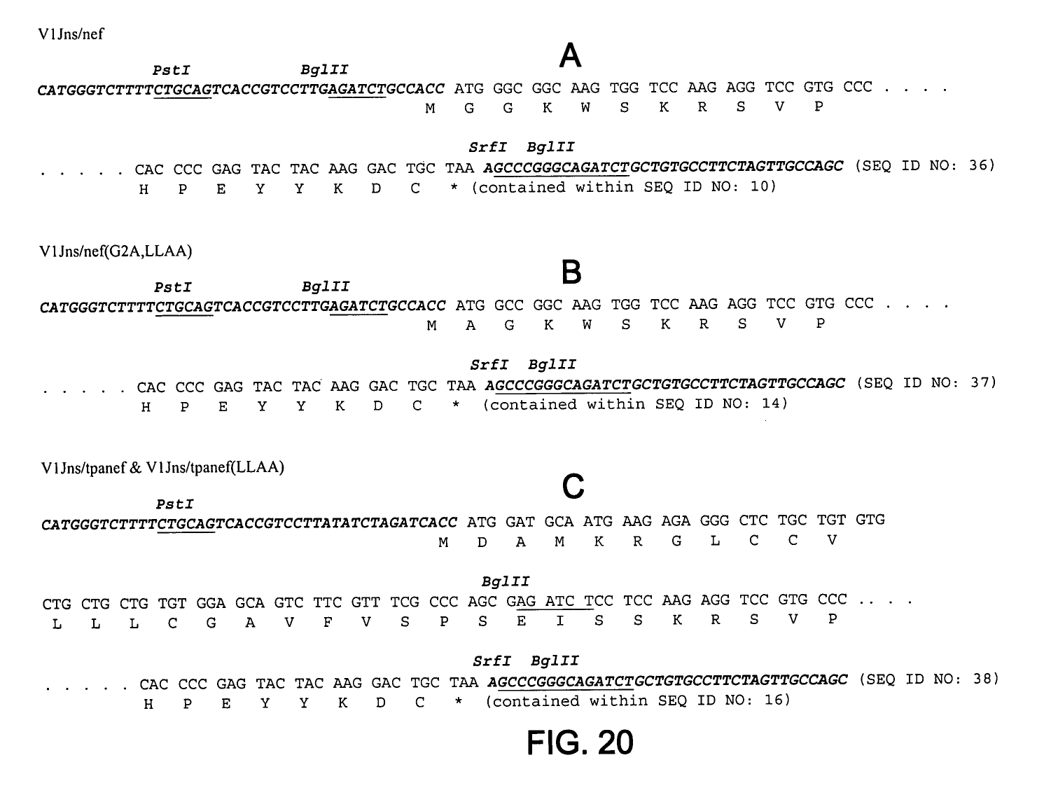

FIG. 20A-C show nucleotide sequences at junctions between nef coding sequence and plasmid backbone of nef expression vectors V1Jns/nef (FIG. 20A), V1Jns/nef(G2A,LLAA) (FIG. 20B), V1Jns/tpanef (FIG. 20C) and V1Jns/tpanef(LLAA) (FIG. 20C, also). 5′ and 3′ flanking sequences of codon optimized nef or codon optimized nef mutant genes are indicated by bold/italic letters; nef and nef mutant coding sequences are indicated by plain letters. Also indicated (as underlined) are the restriction endonuclease sites involved in construction of respective nef expression vectors. V1Jns/tpanef and V1Jns/tpanef(LLAA) have identical sequences at the junctions.

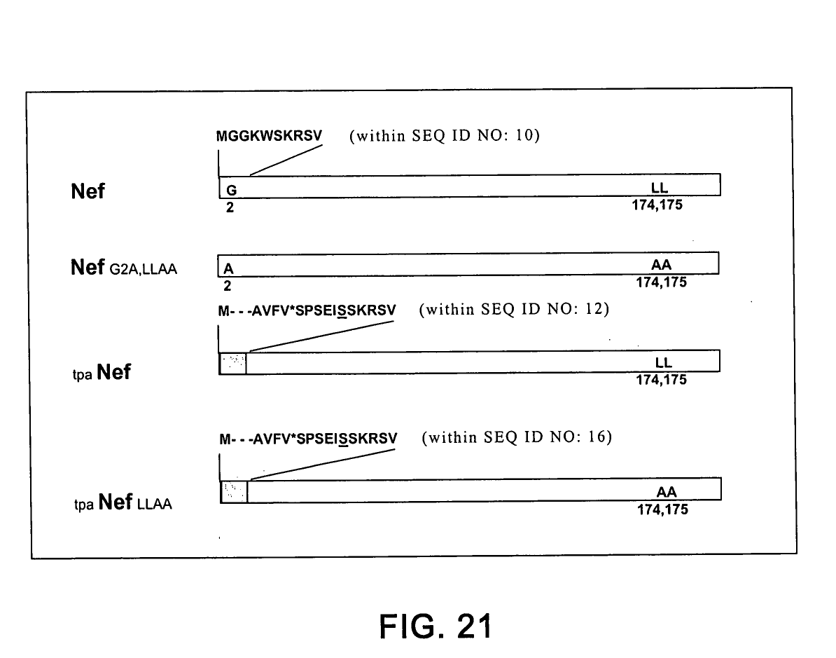

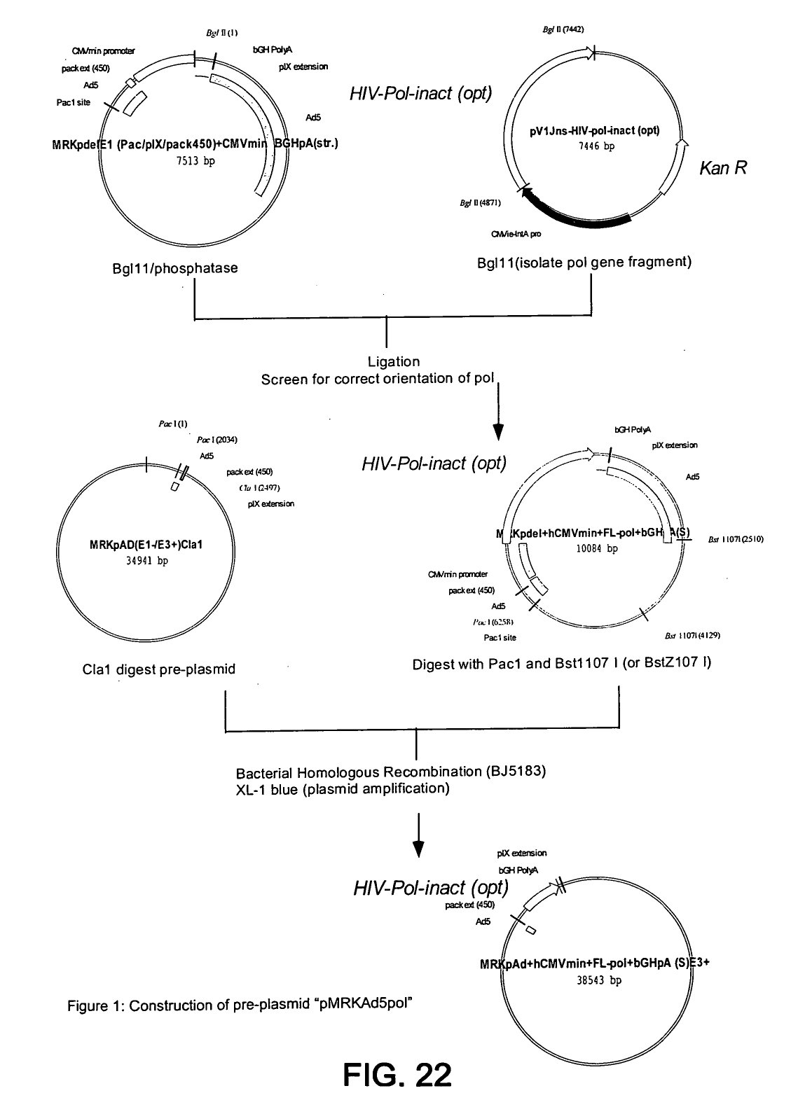

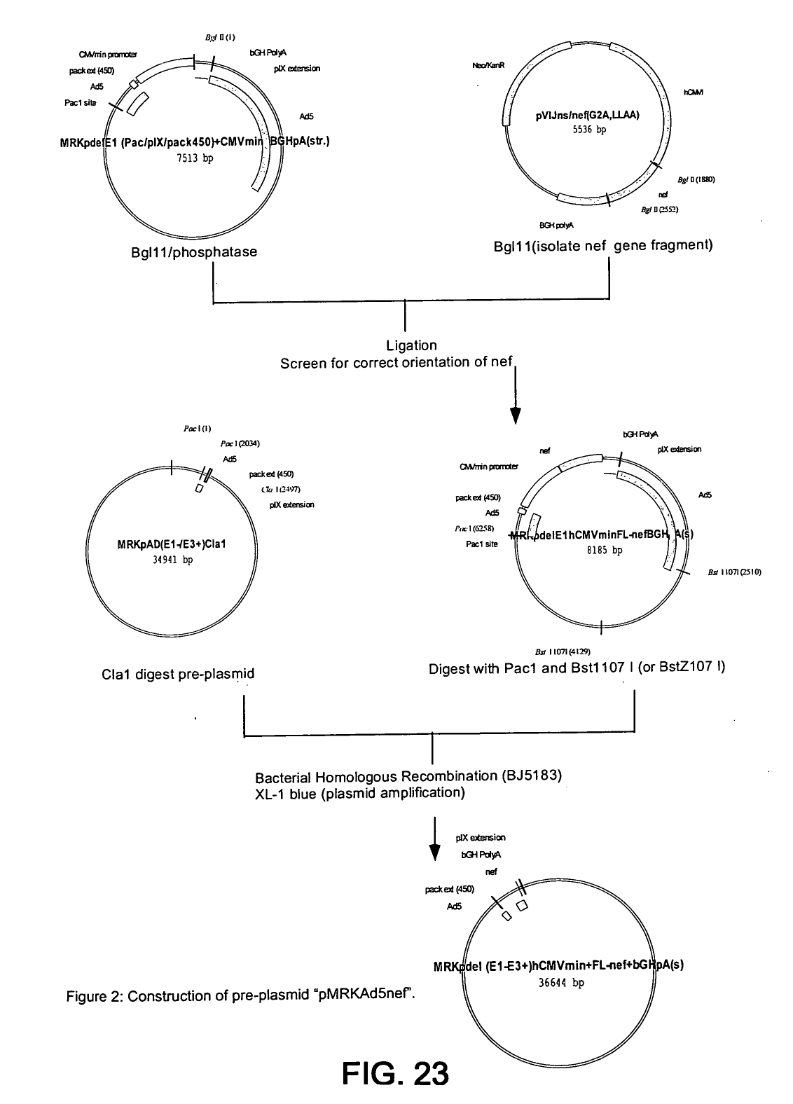

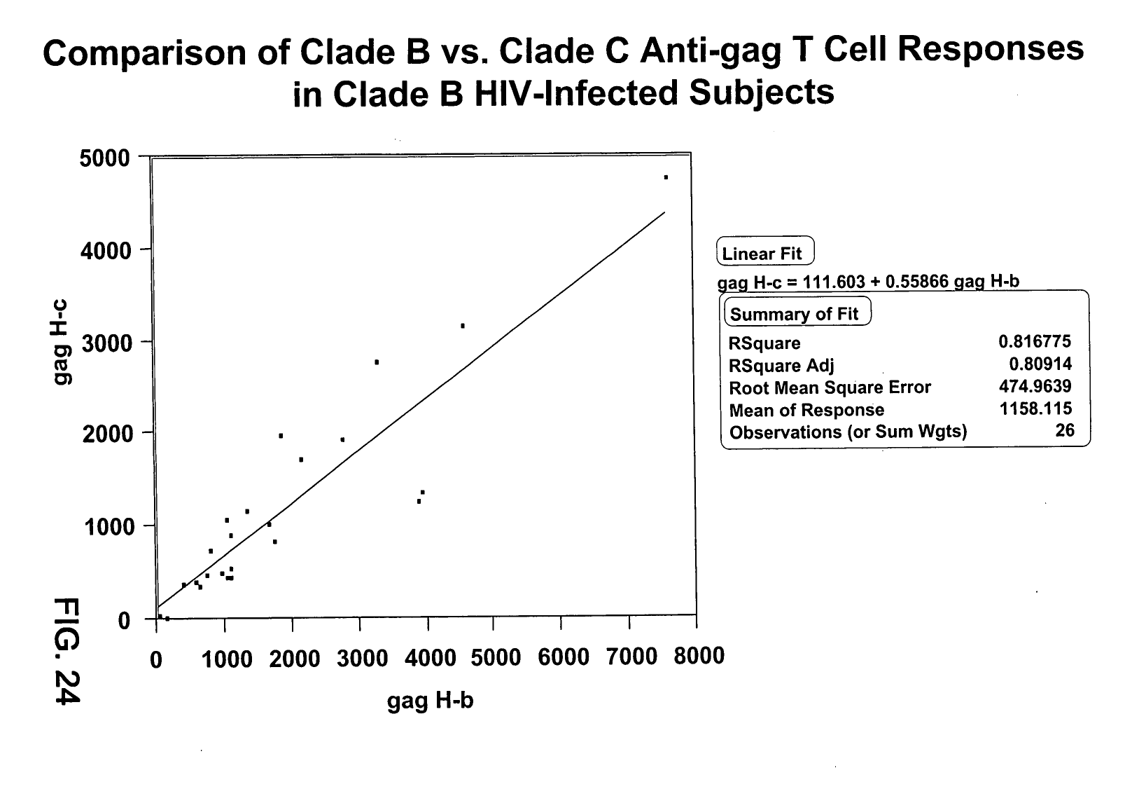

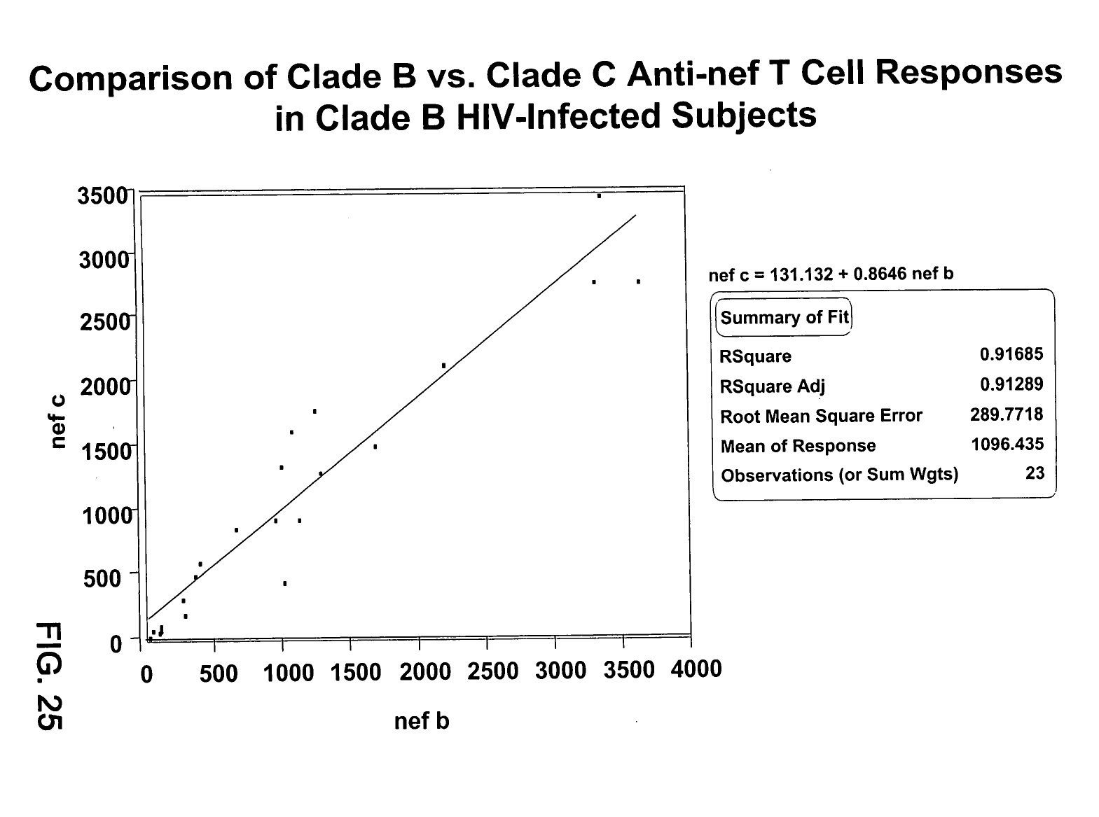

FIG. 21 shows a schematic presentation of nef and nef derivatives. Amino acid residues involved in Nef derivatives are presented. Glycine 2 and Leucine 174 and 175 are the sites involved in myristylation and dileucine motif, respectively.

### EXAMPLE 19

### MRKAd5Pol Construction and Virus Rescue

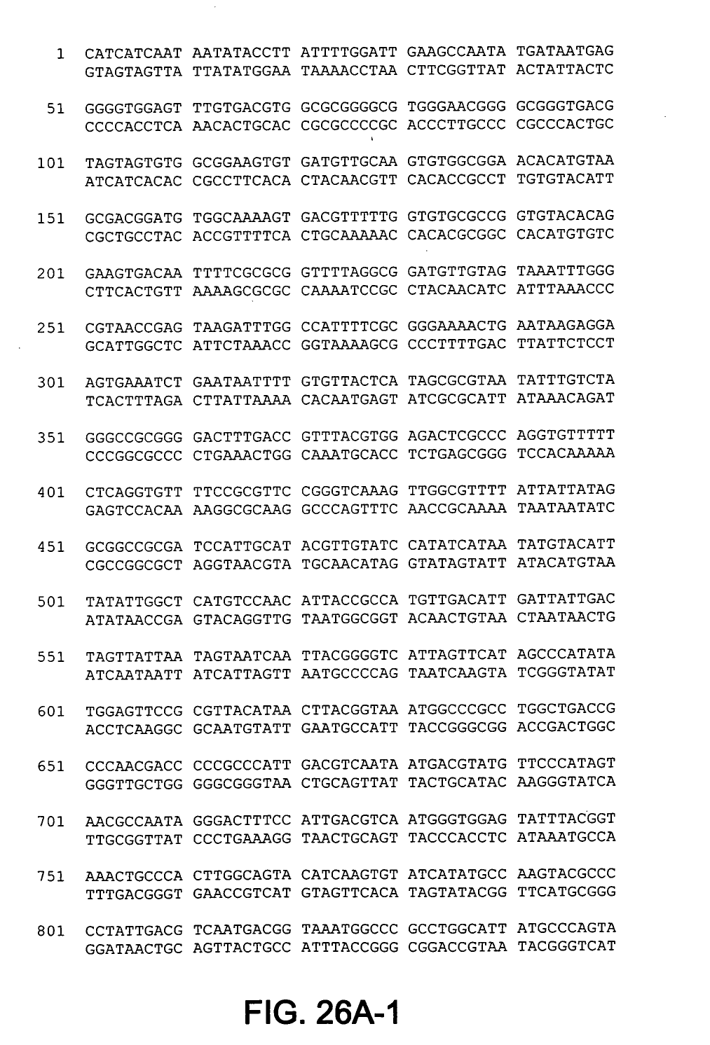

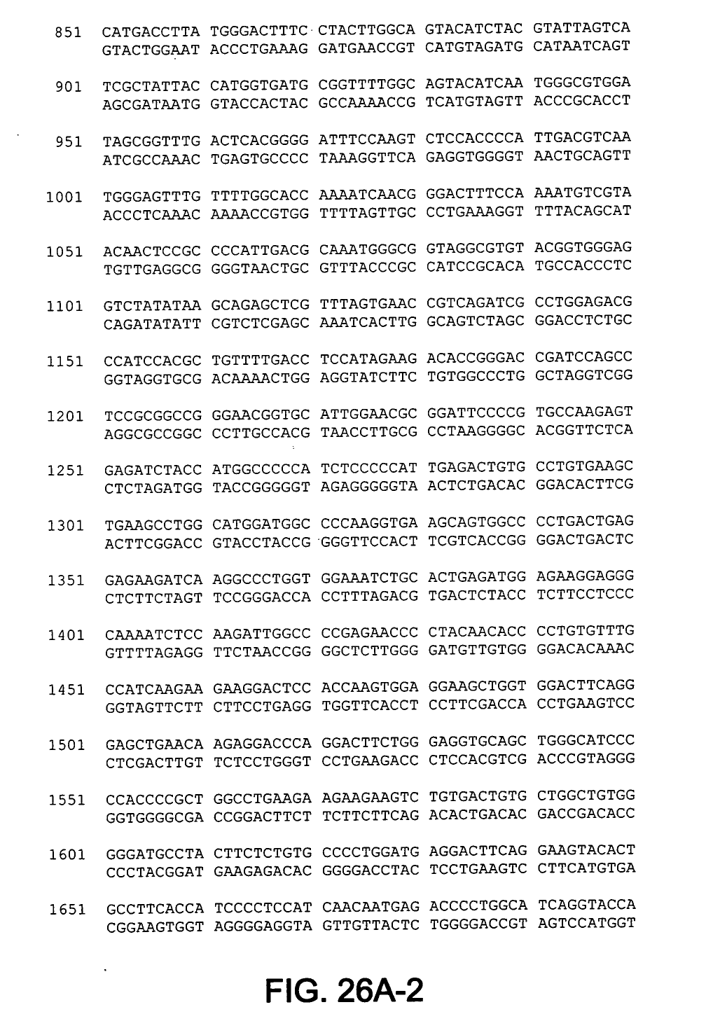

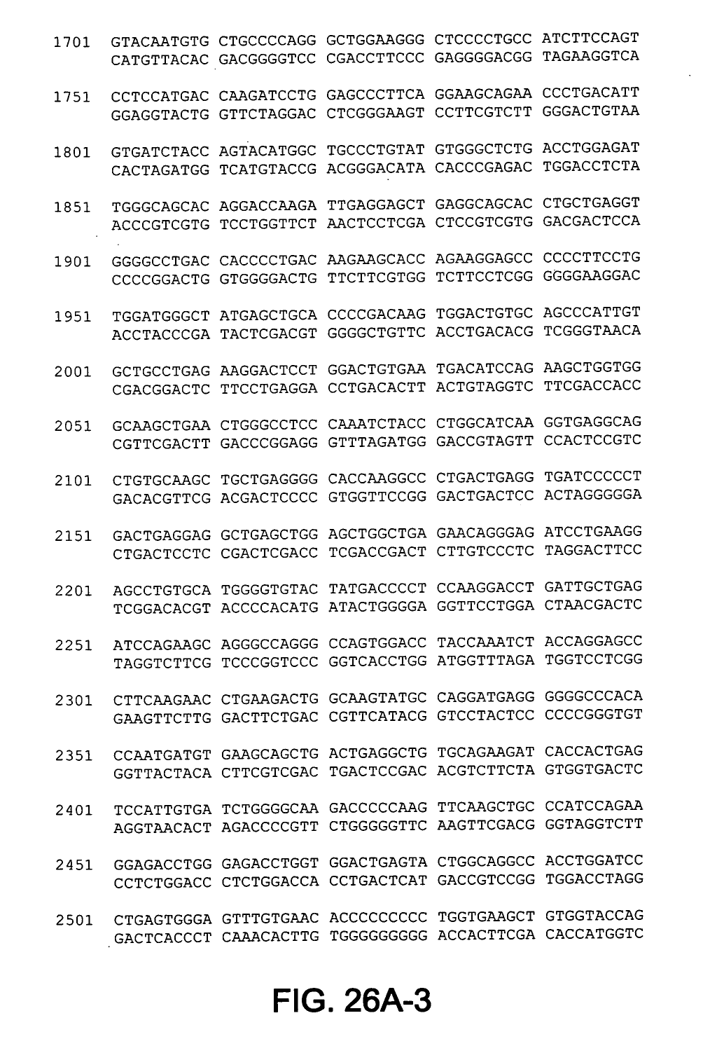

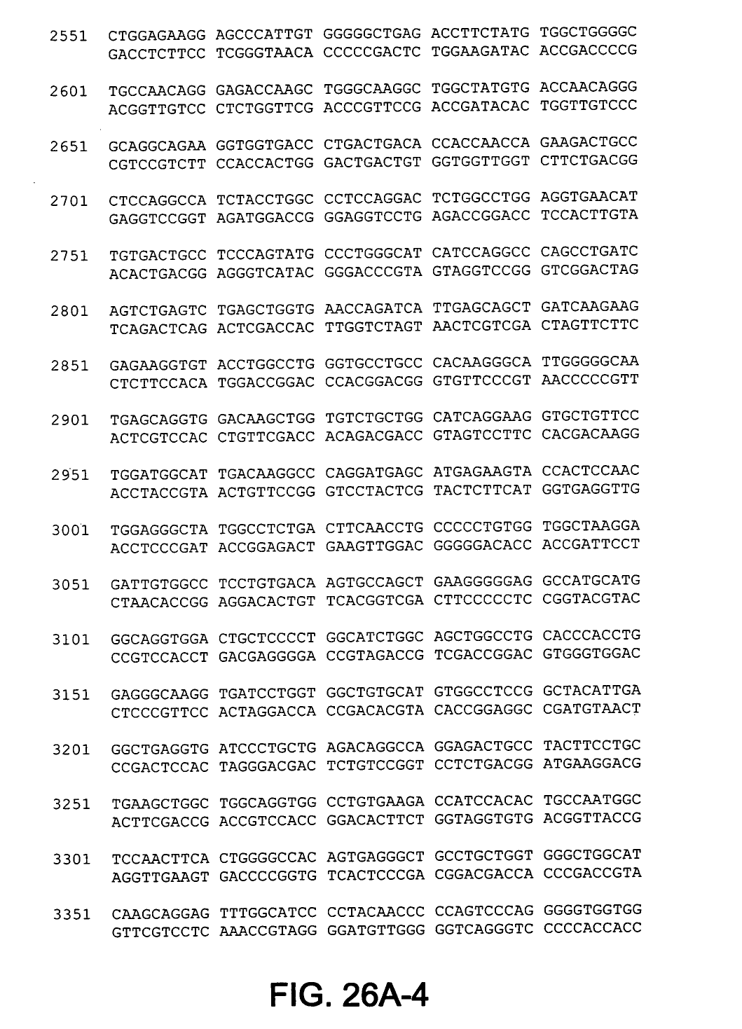

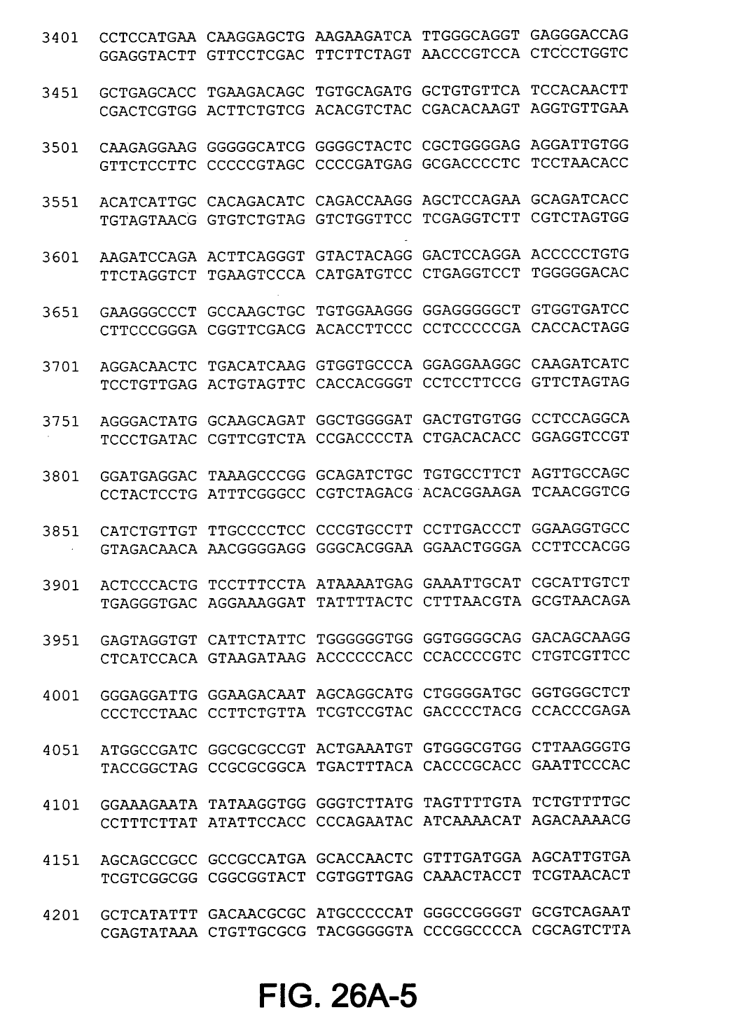

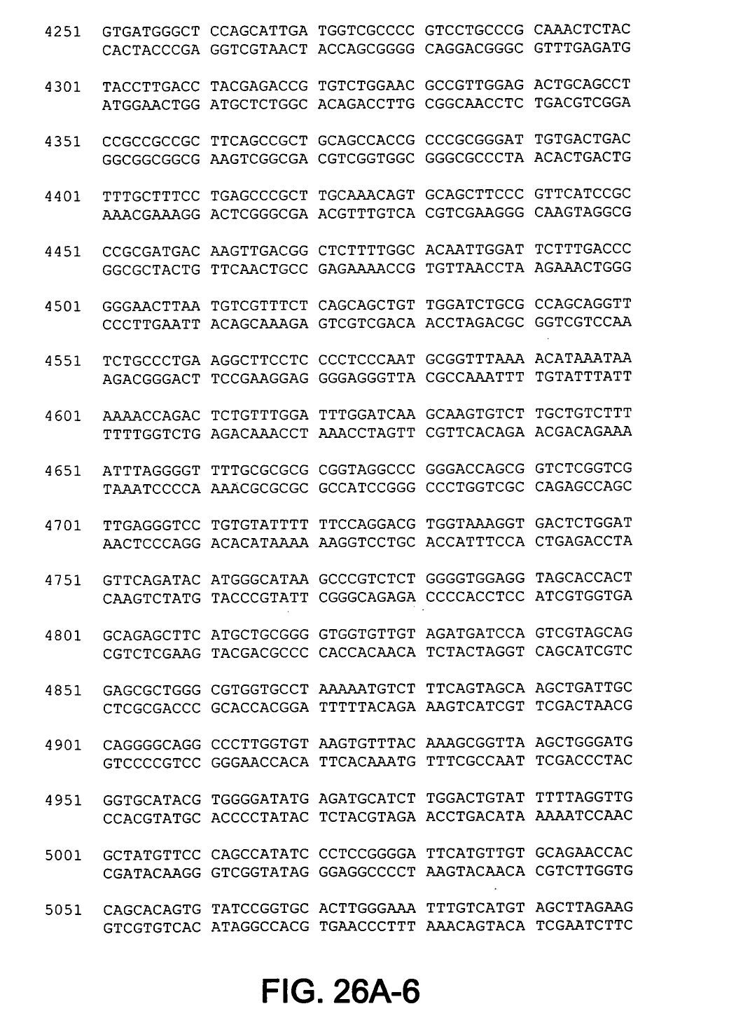

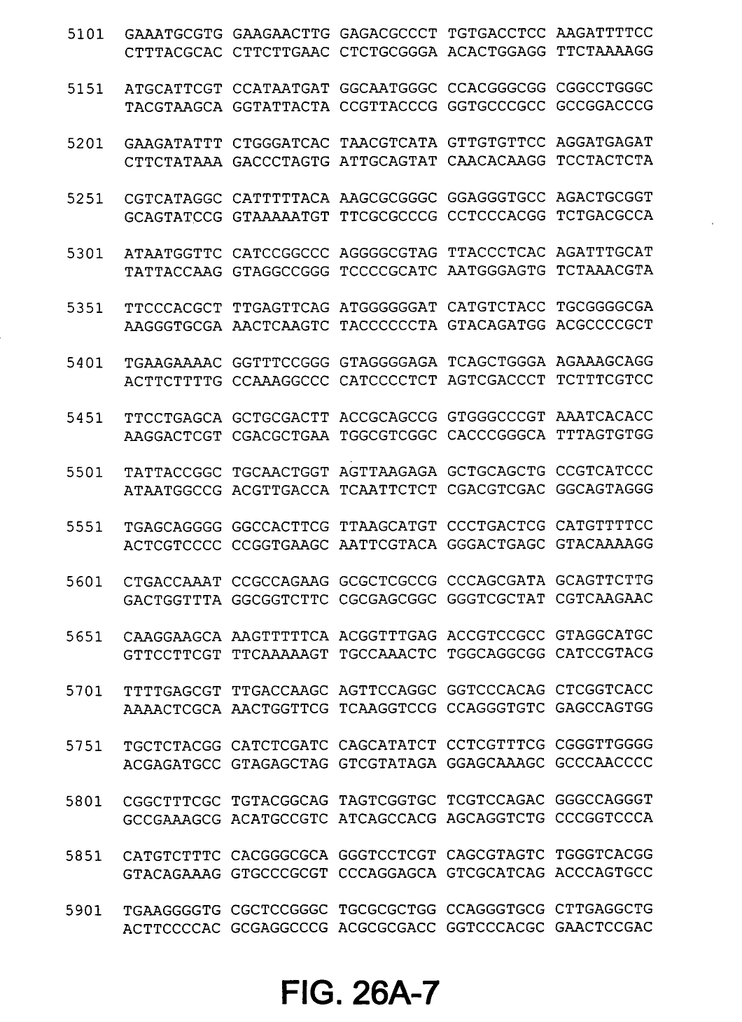

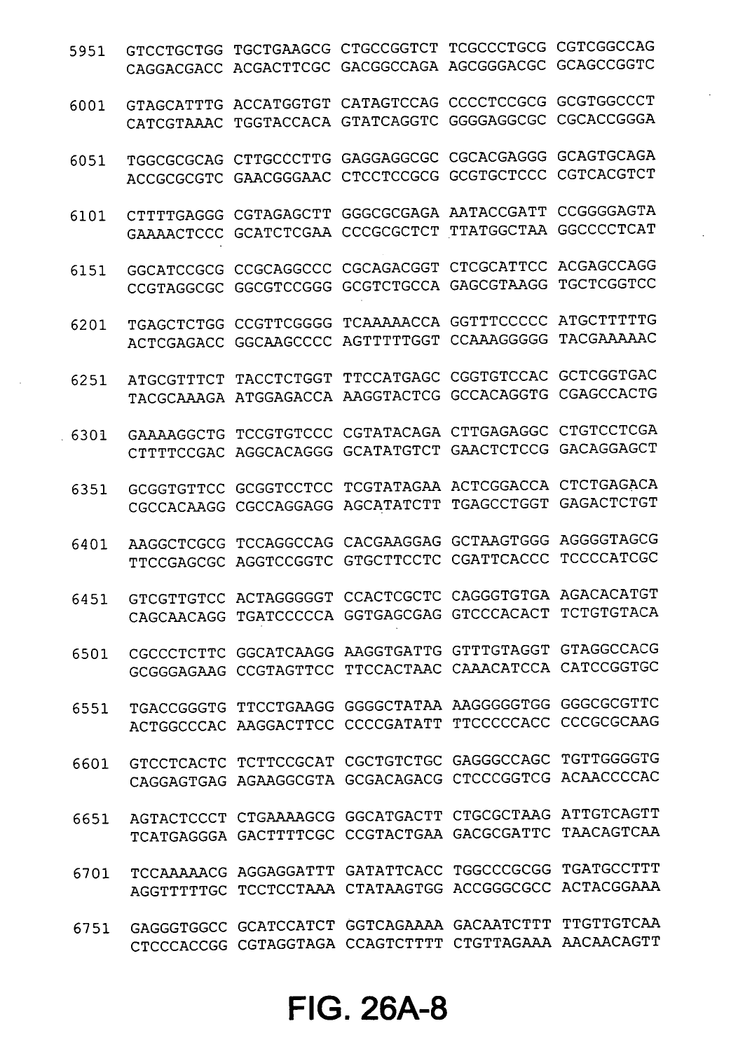

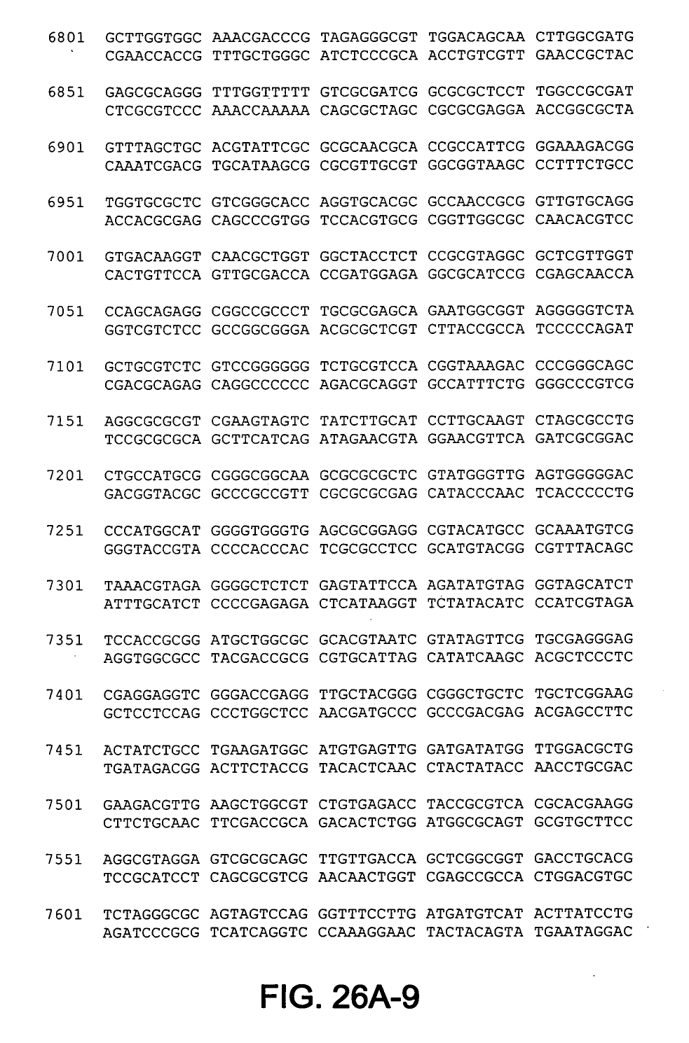

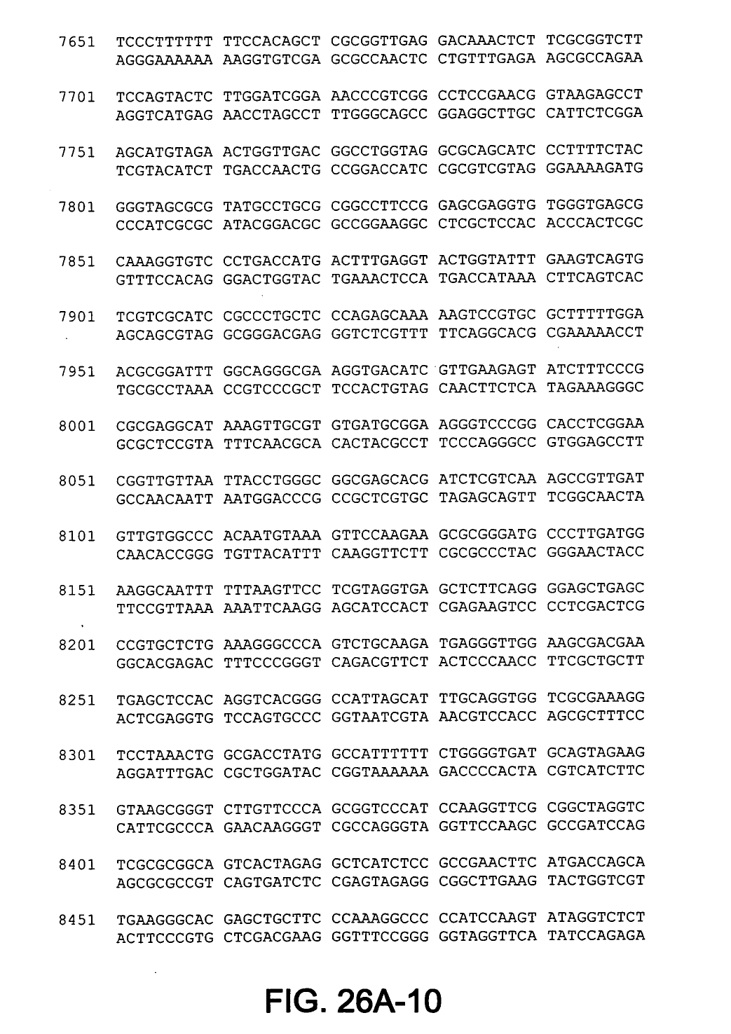

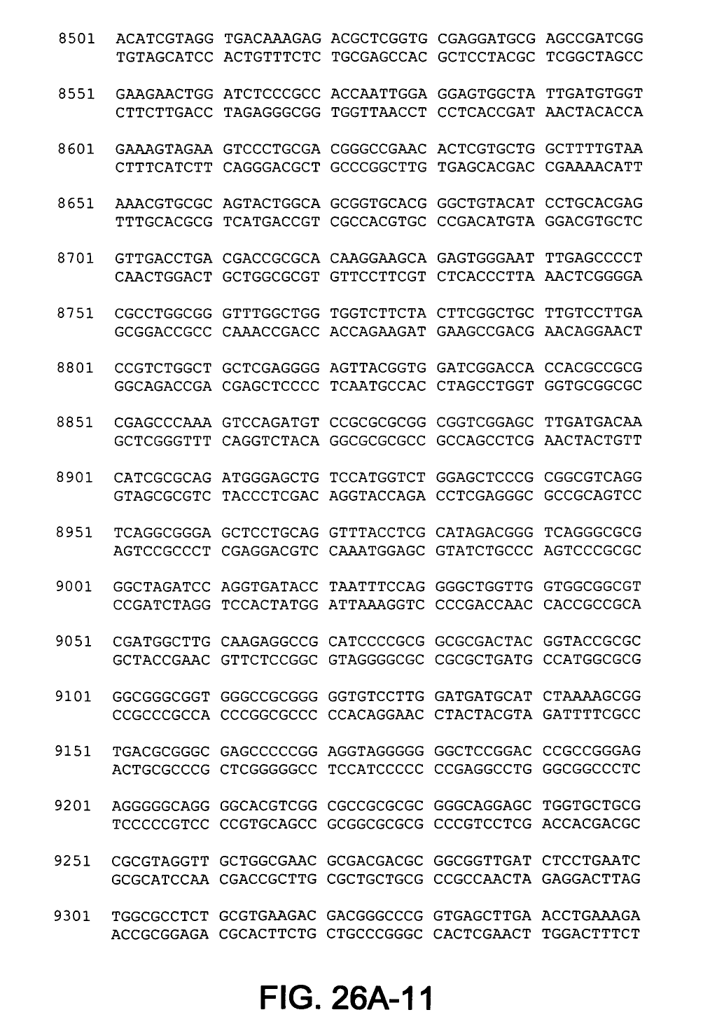

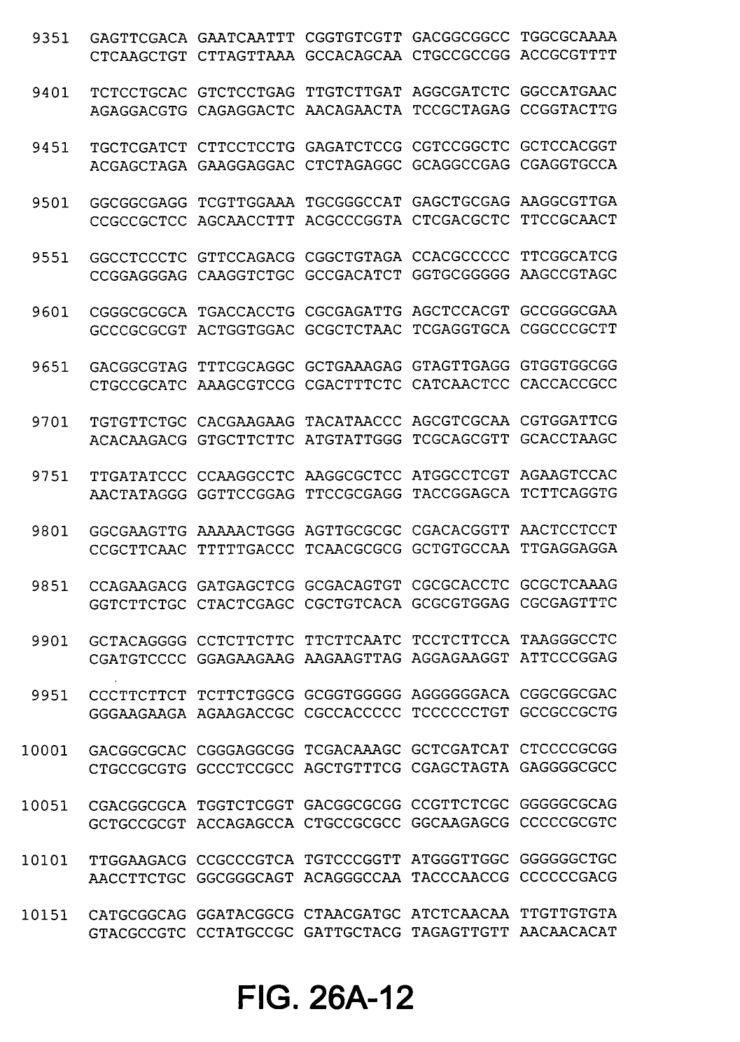

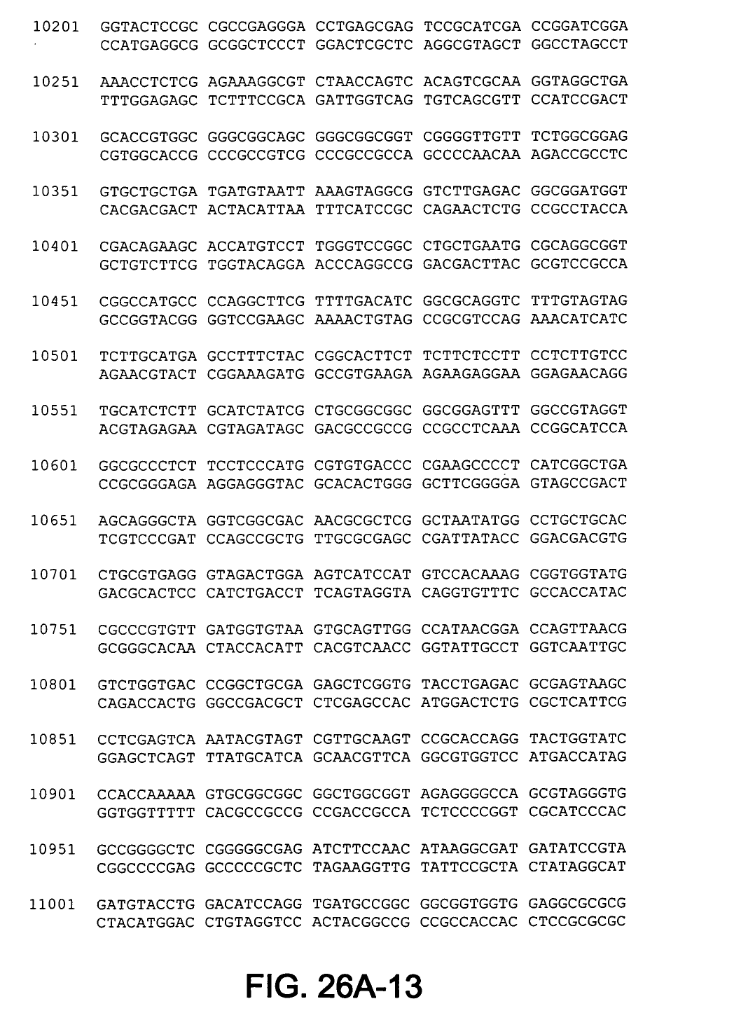

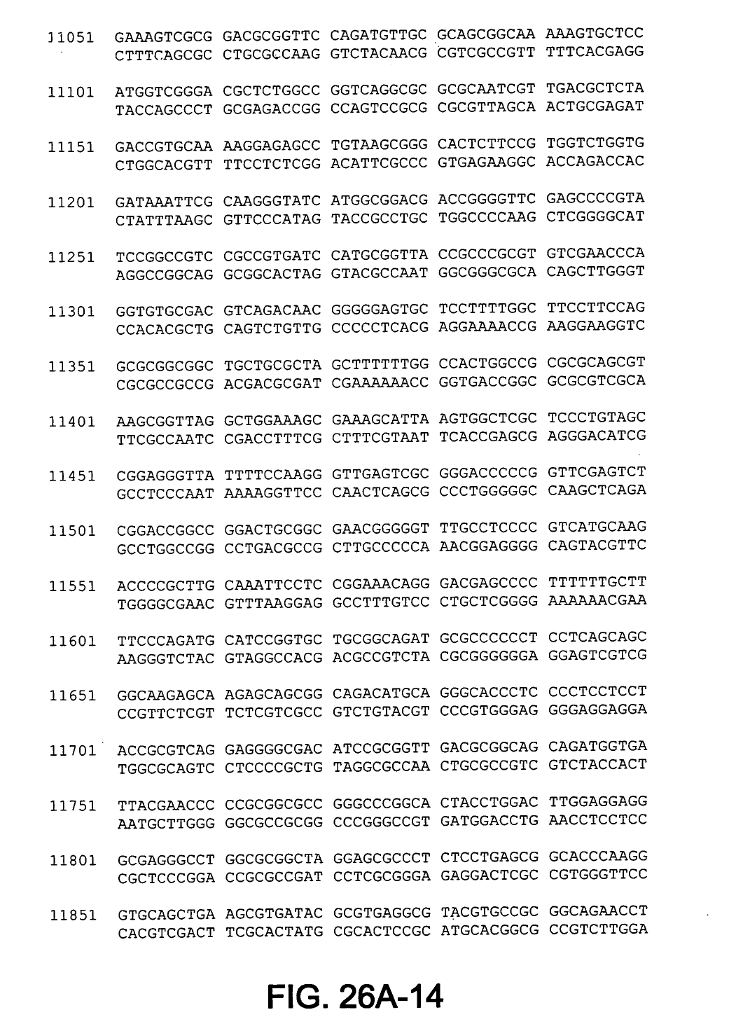

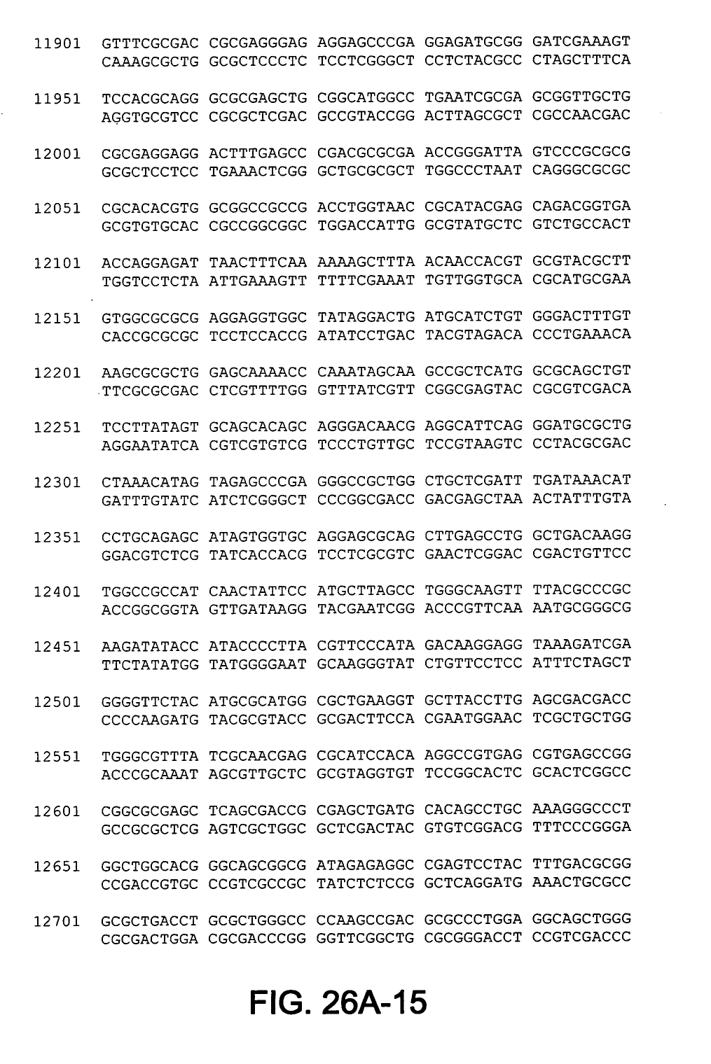

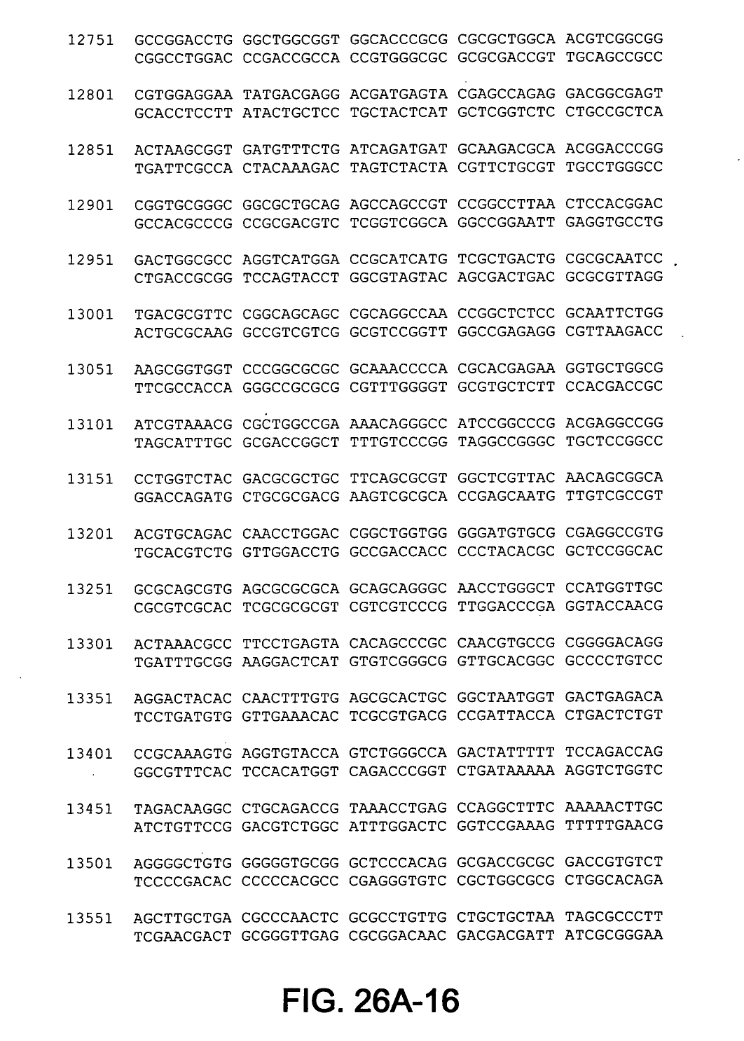

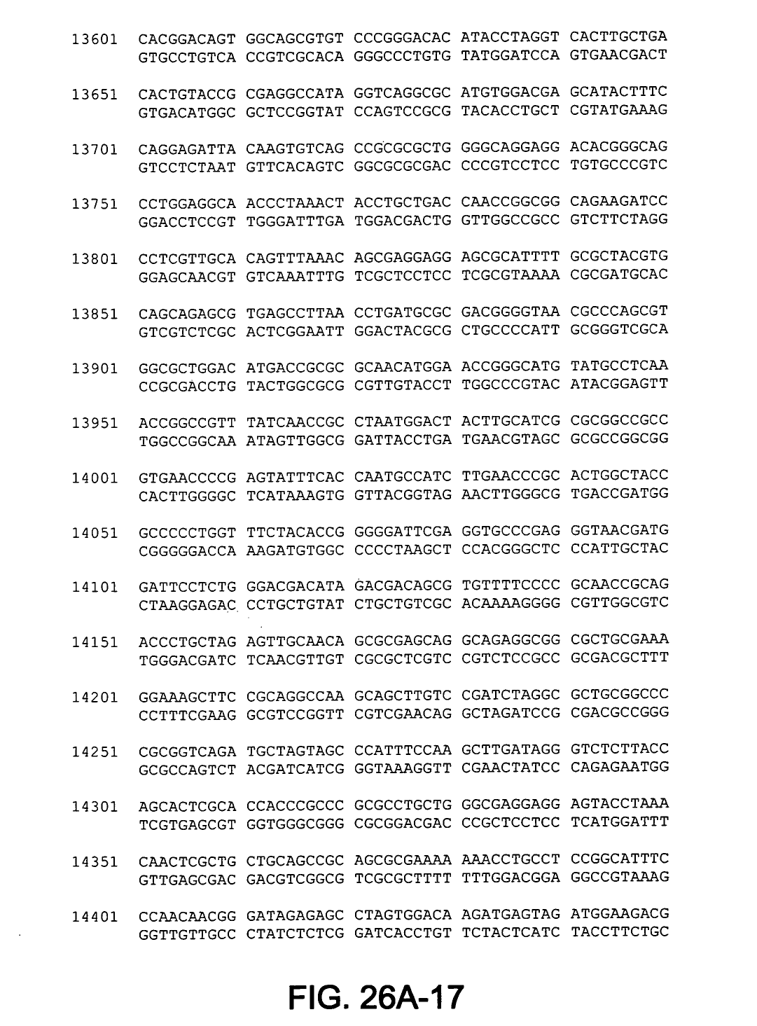

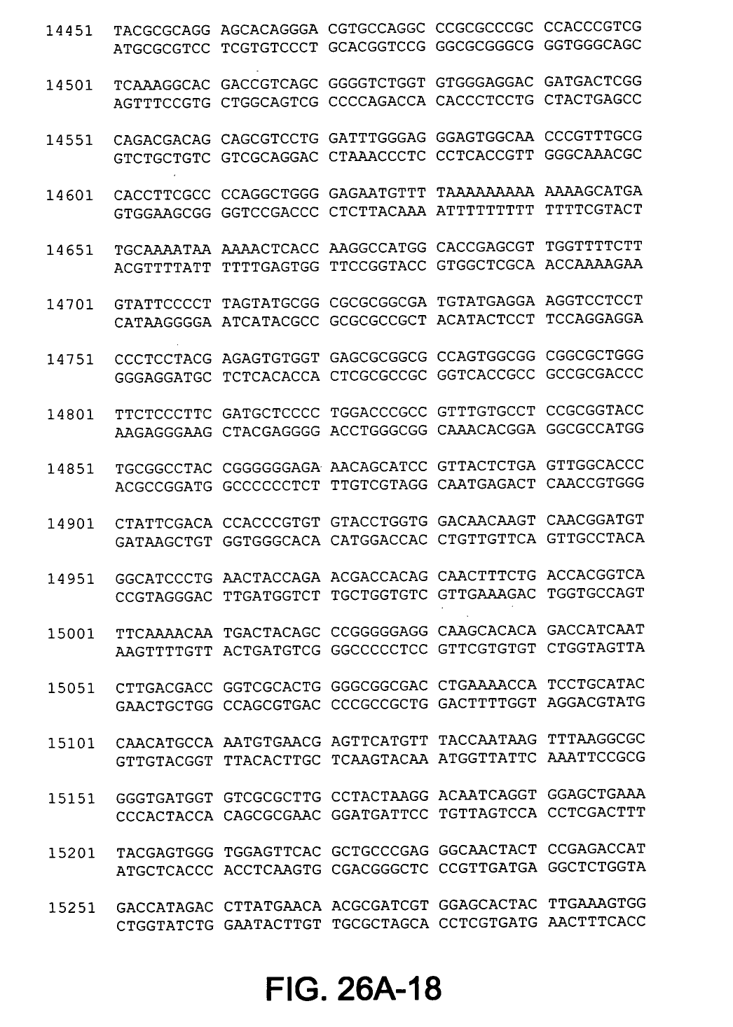

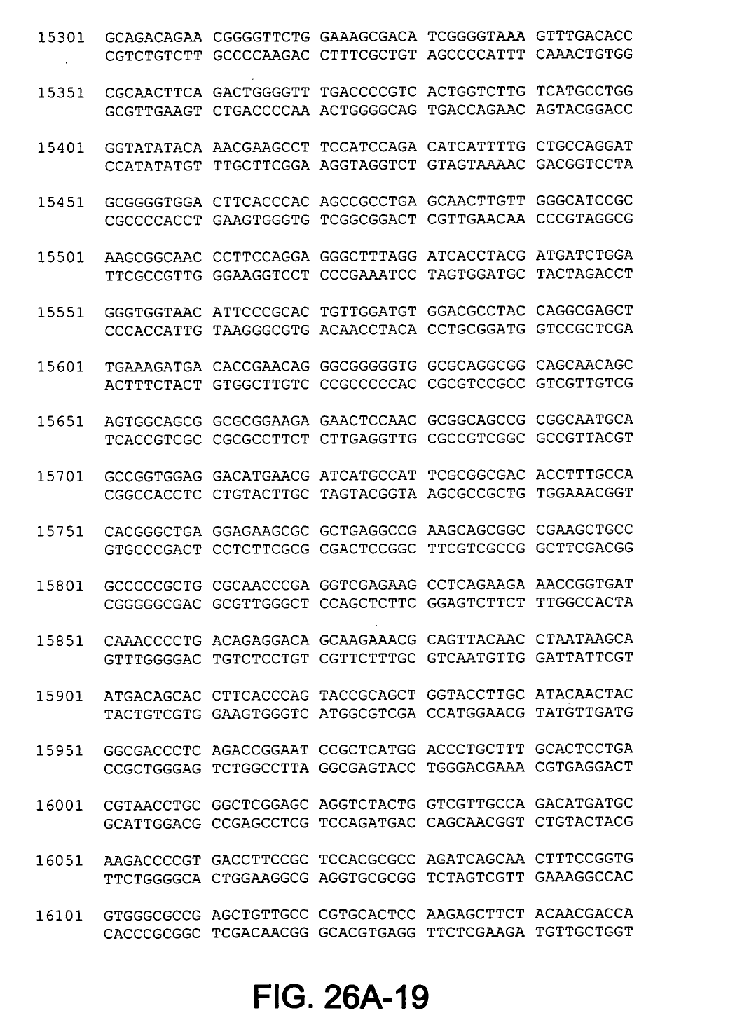

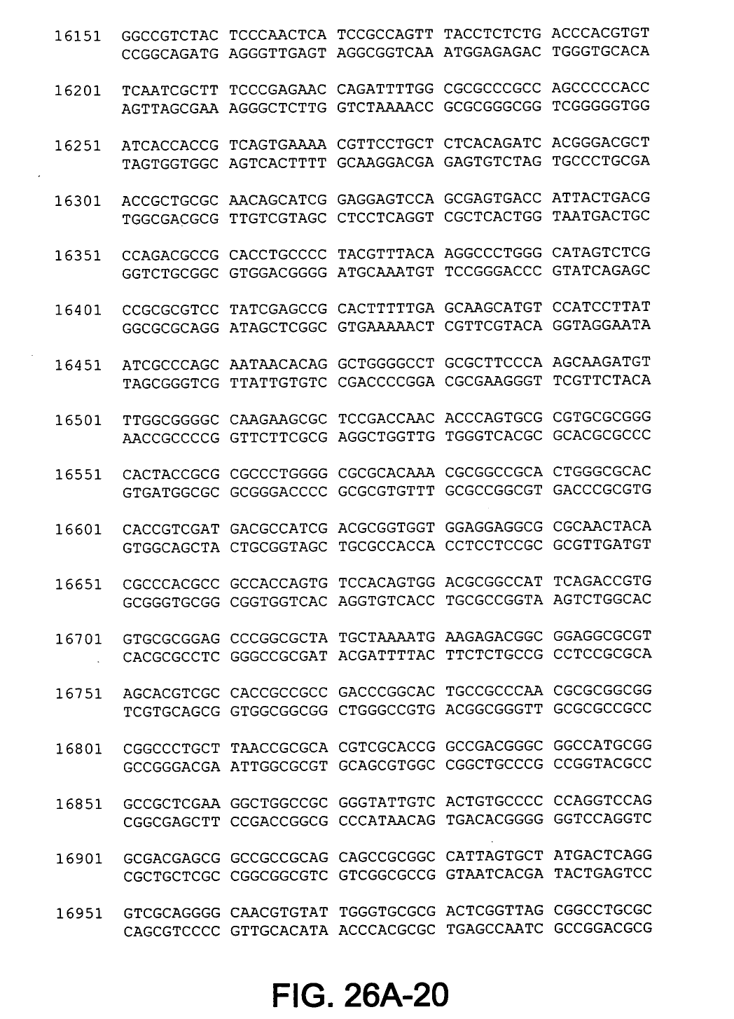

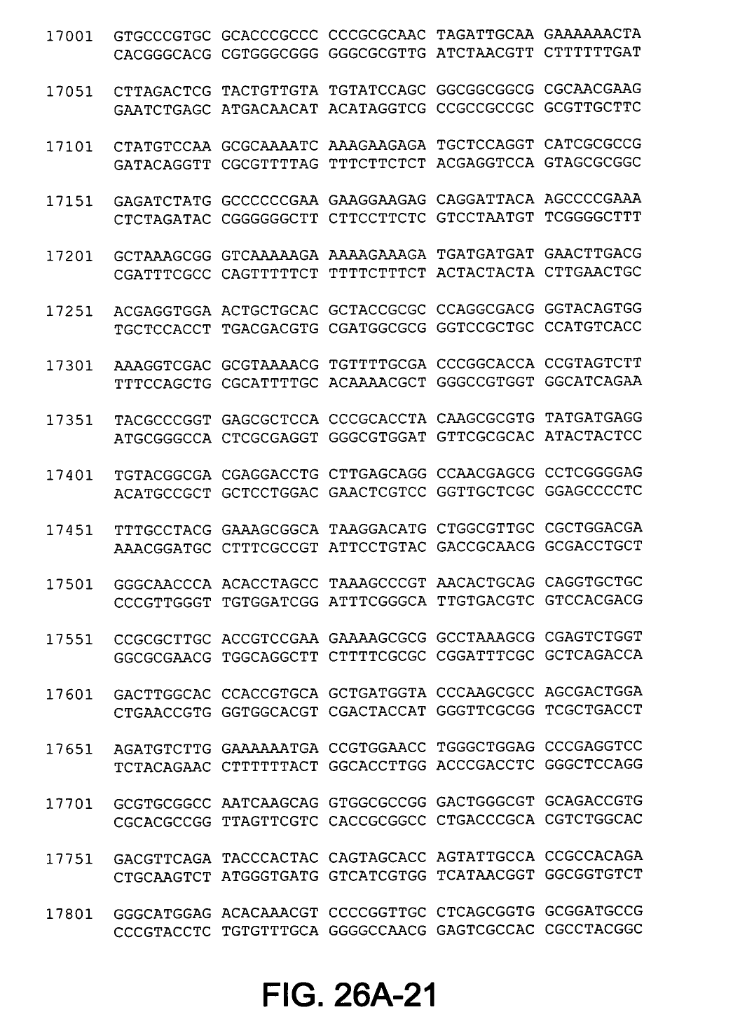

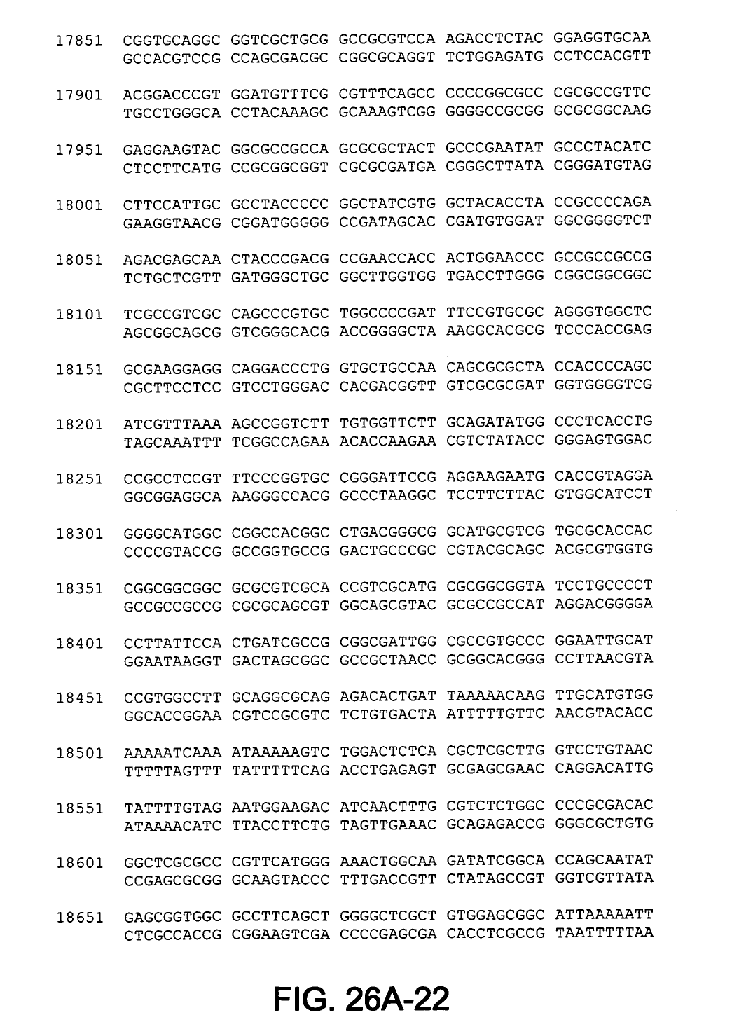

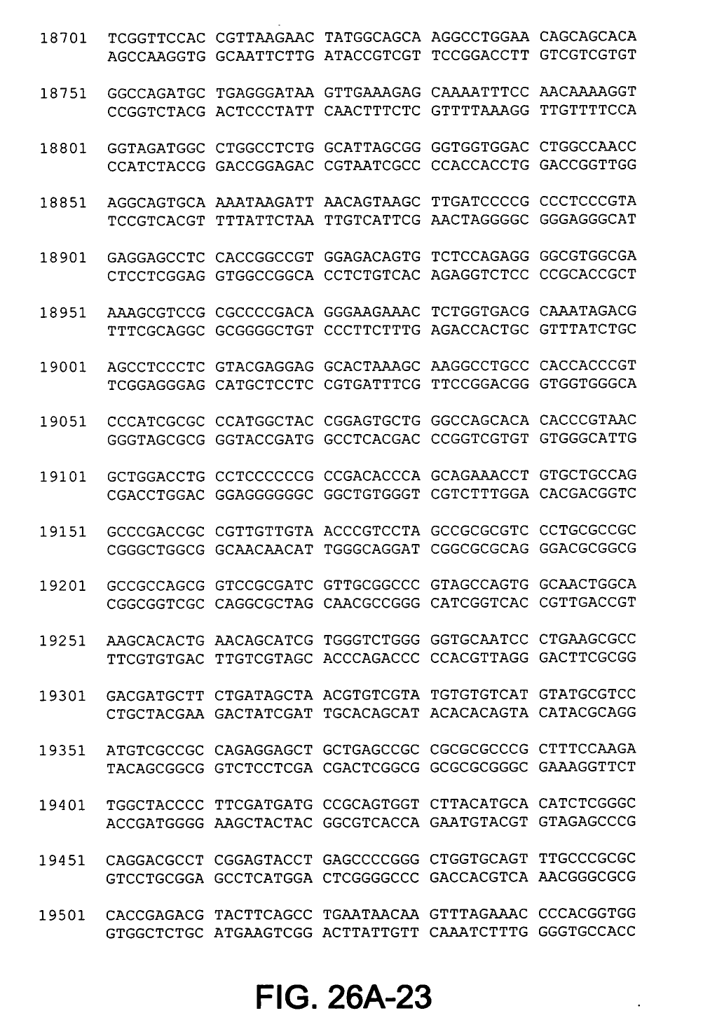

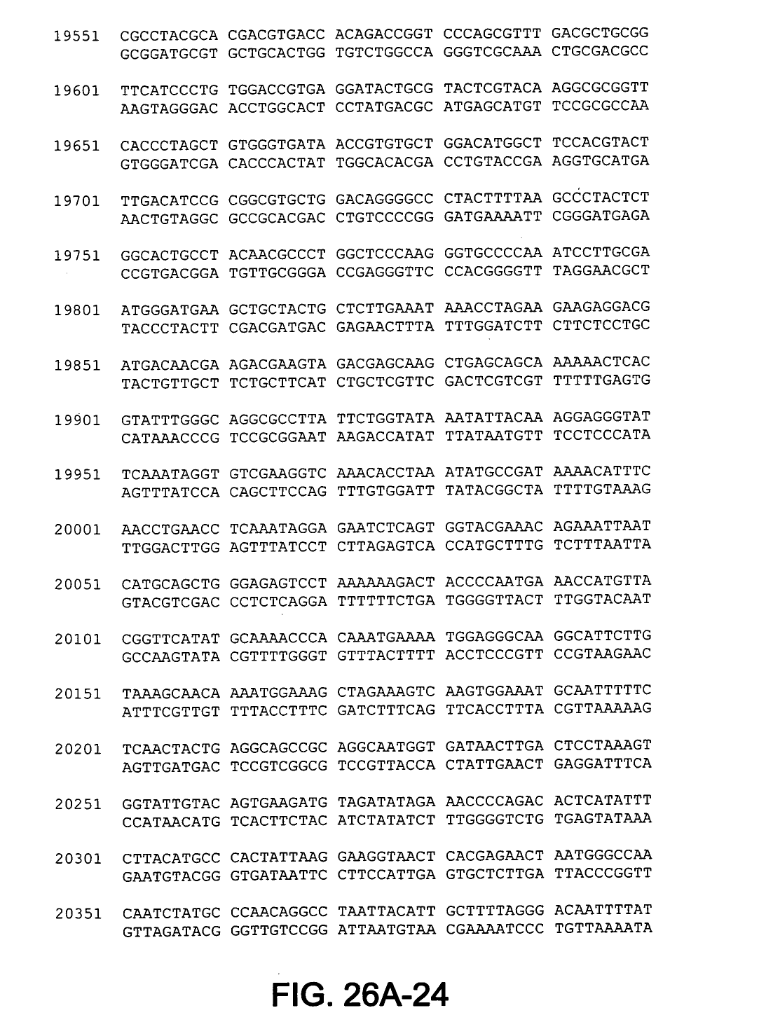

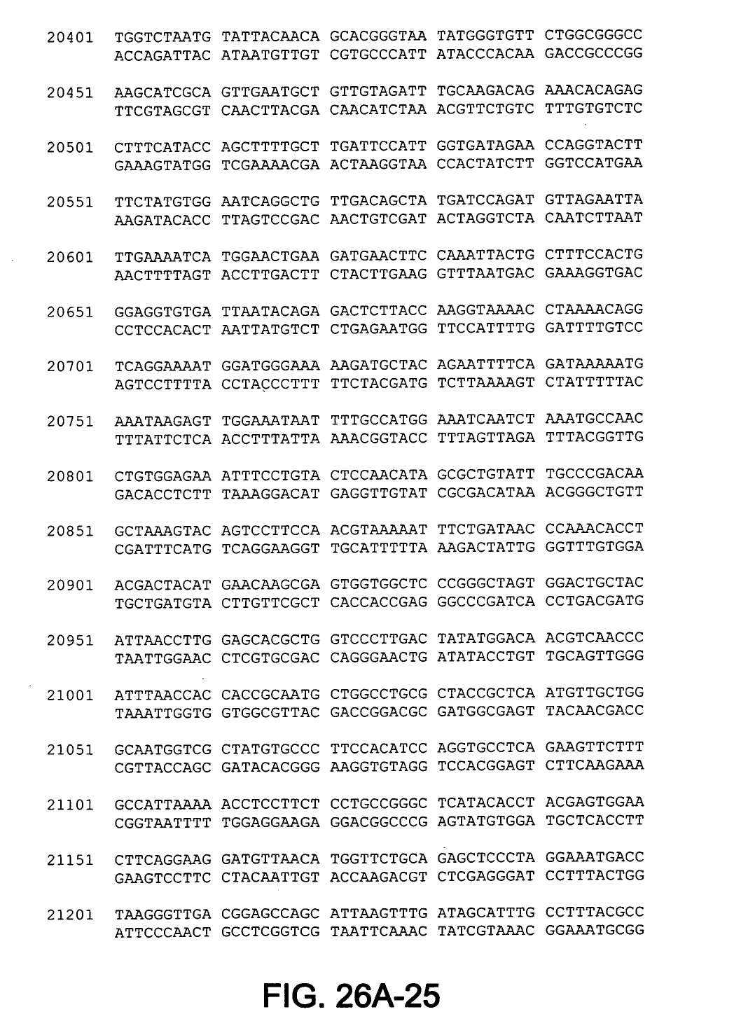

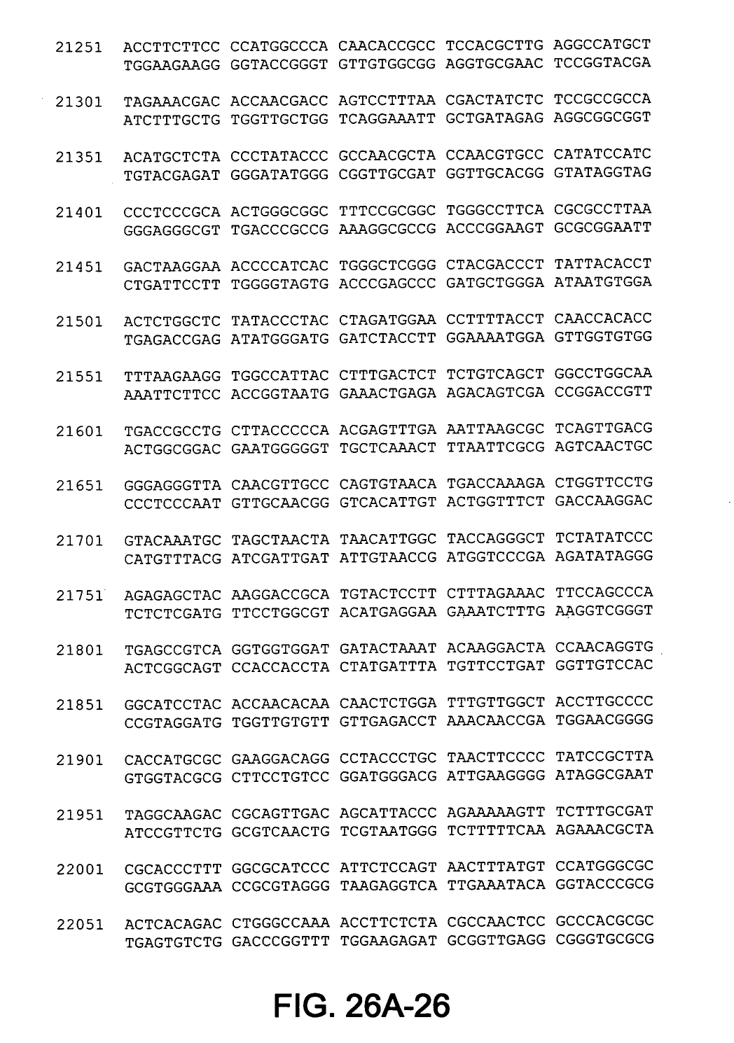

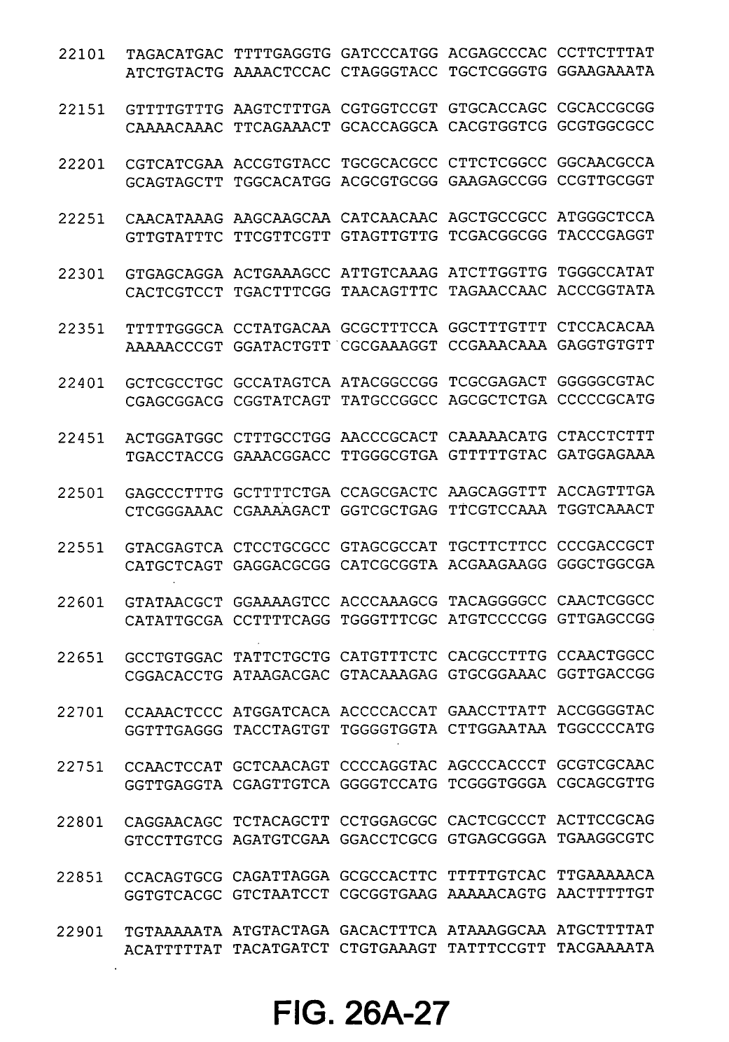

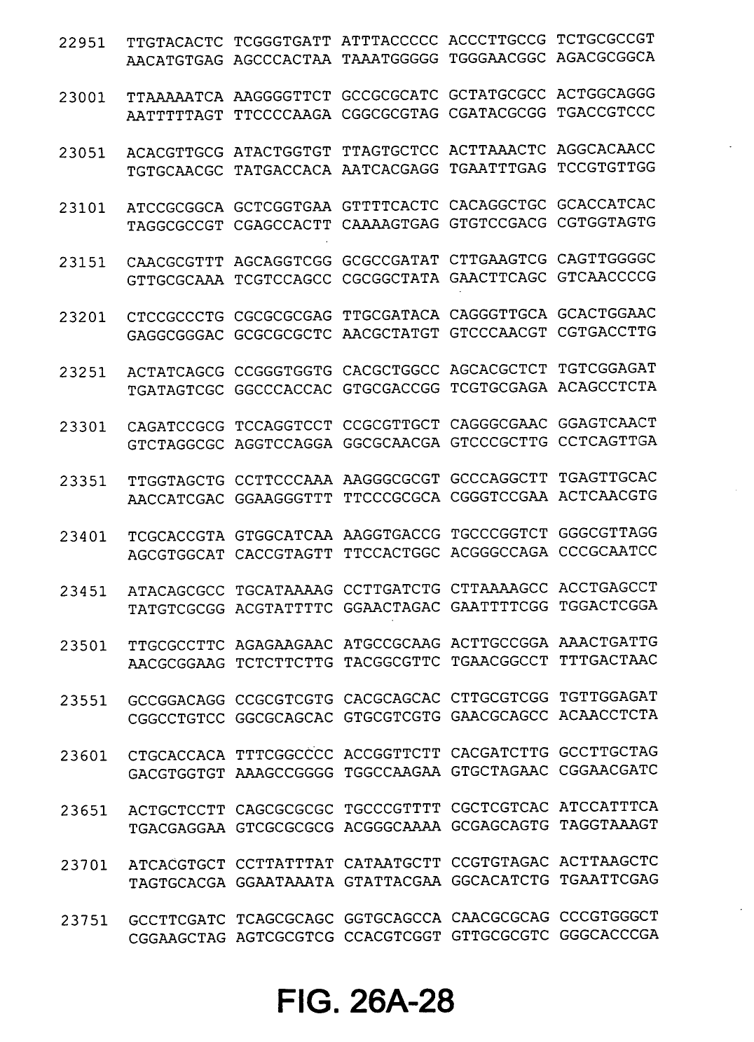

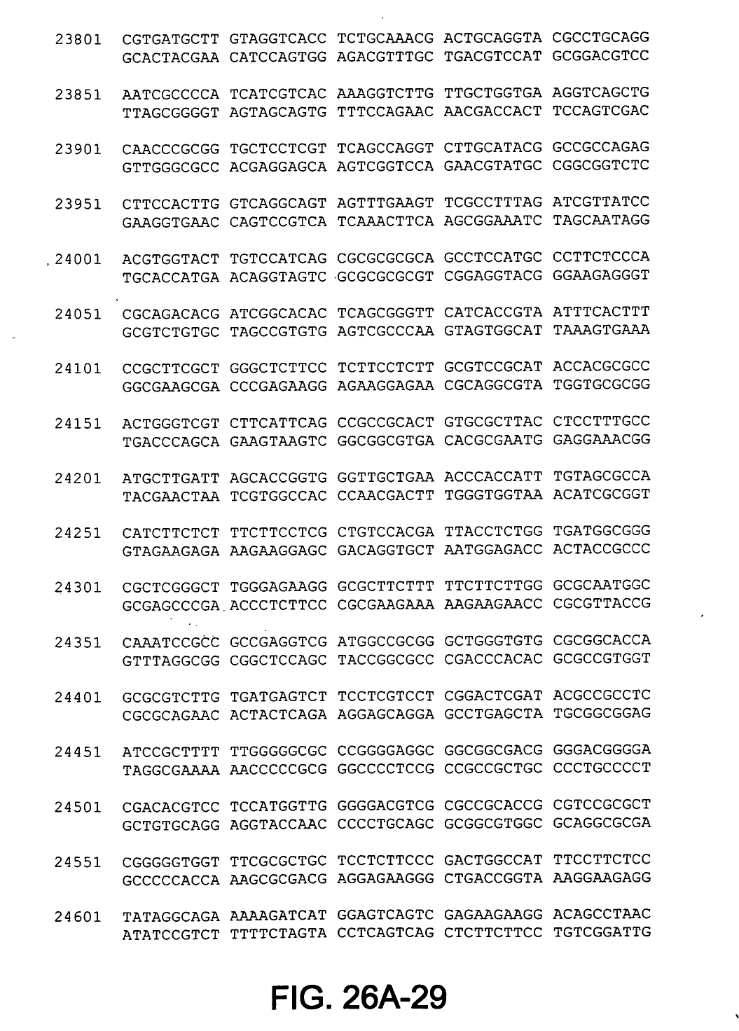

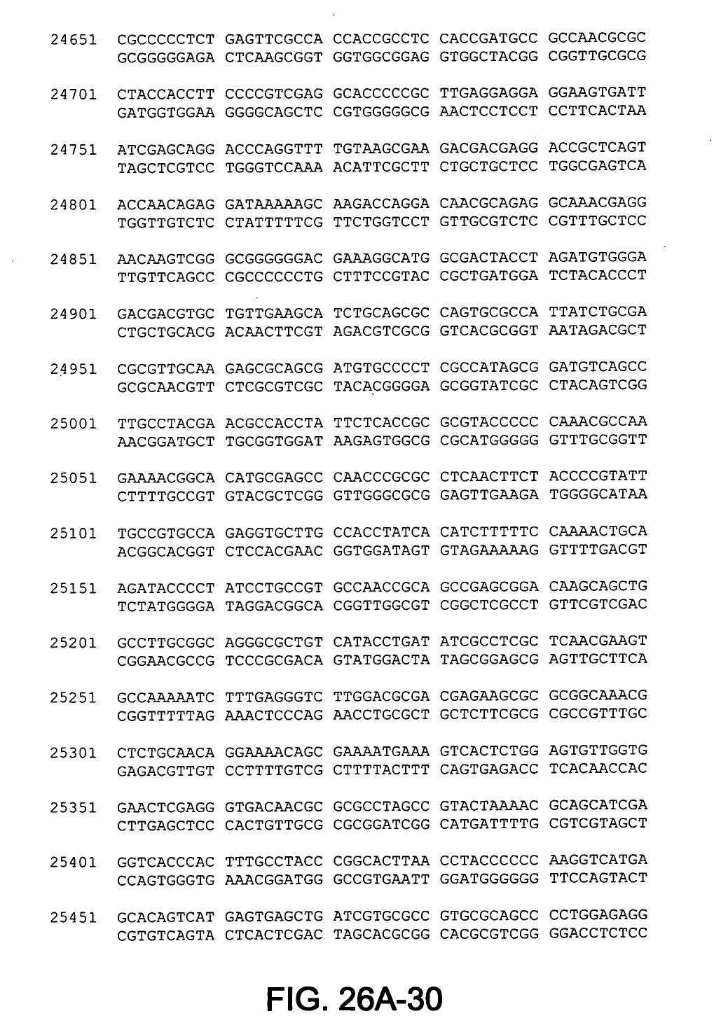

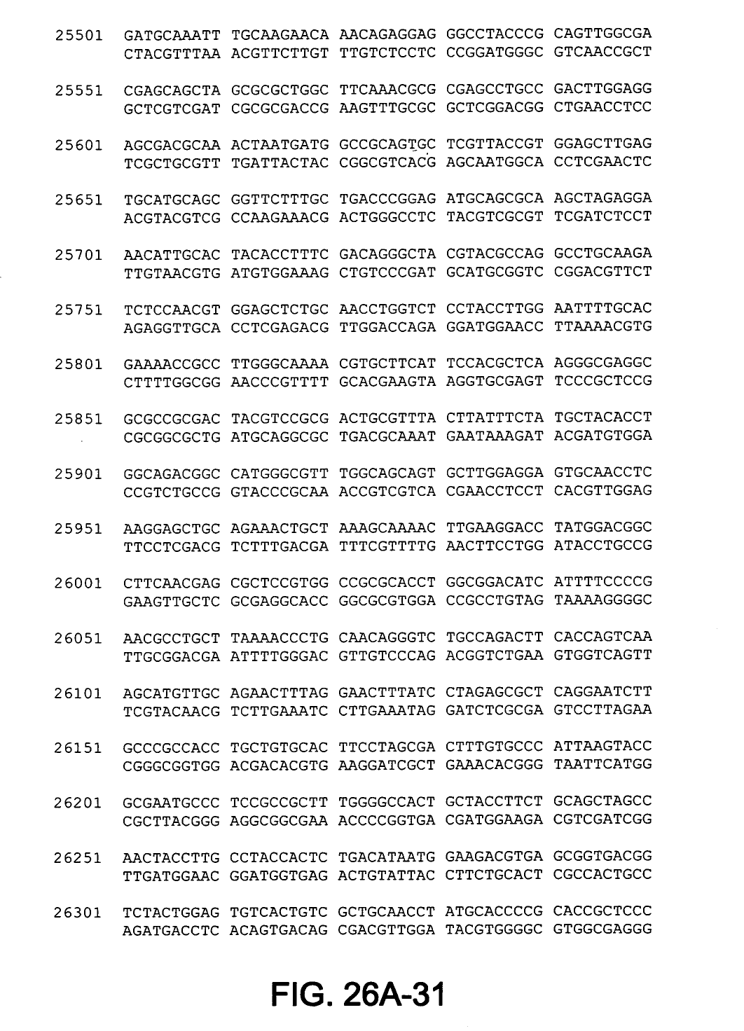

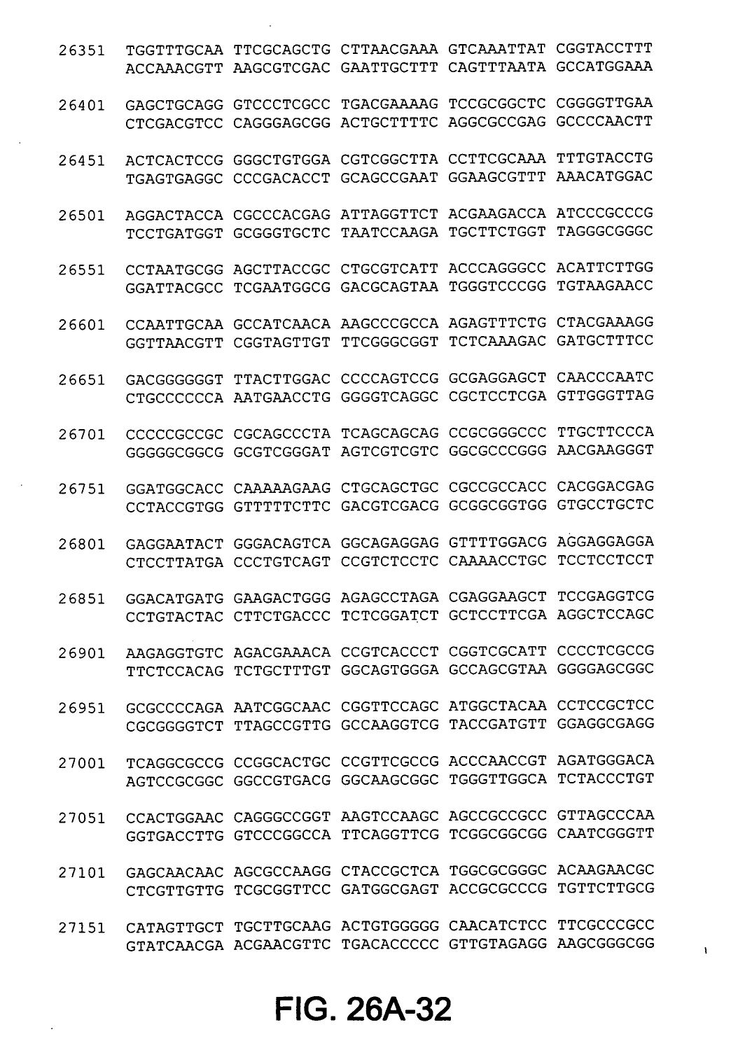

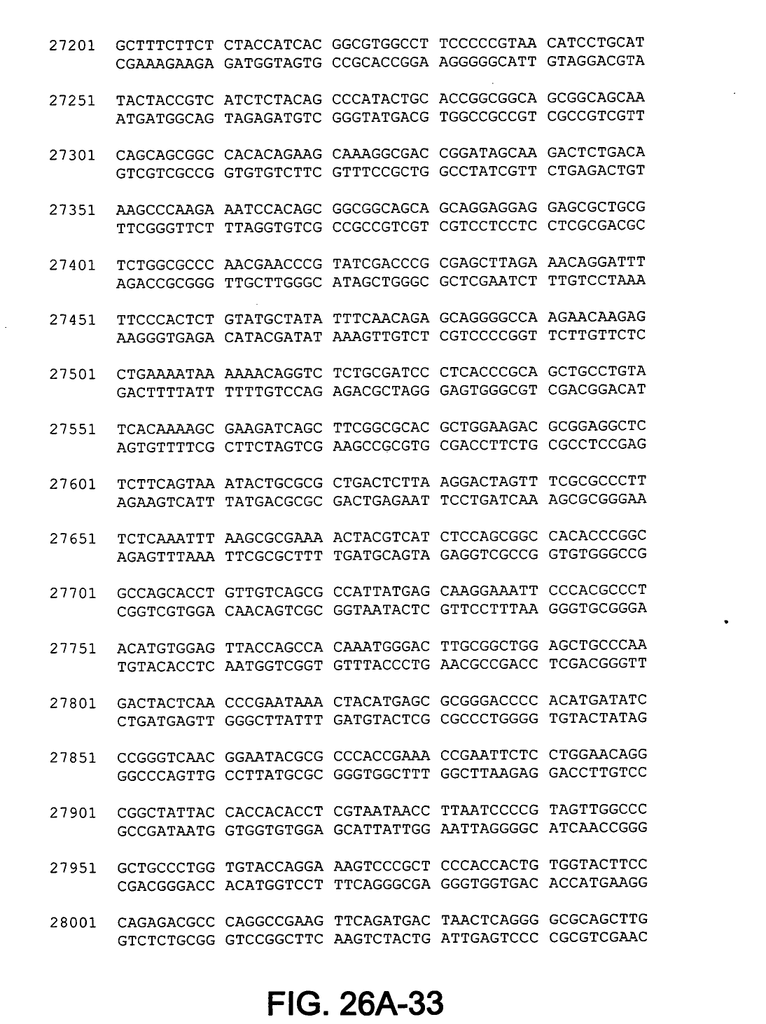

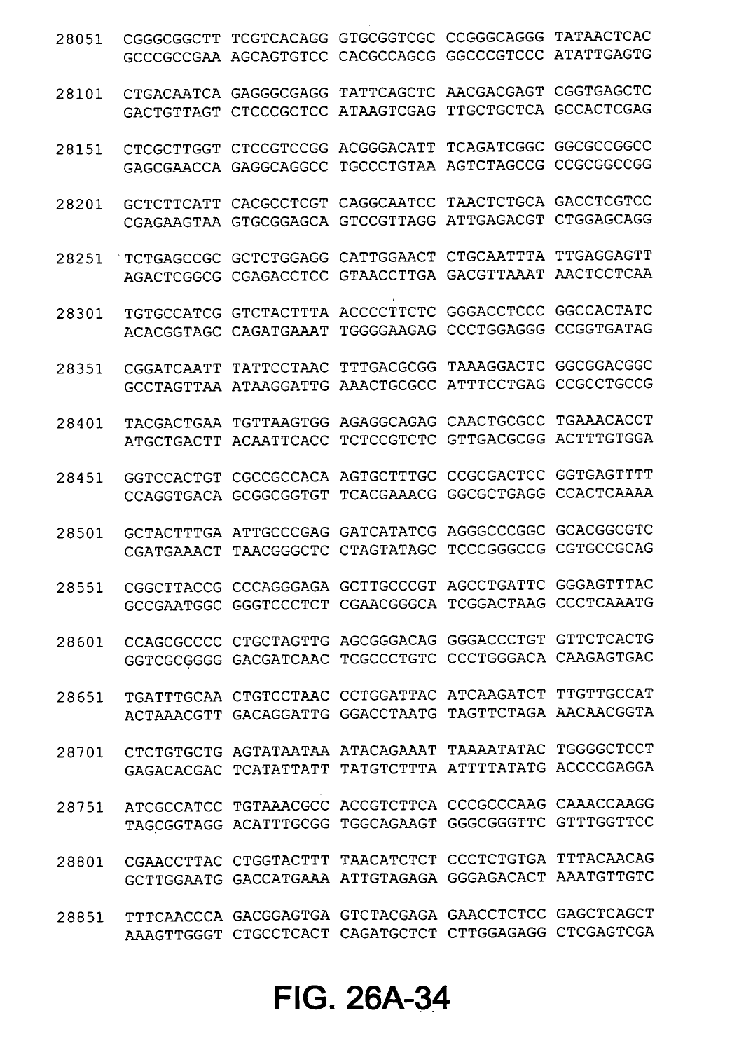

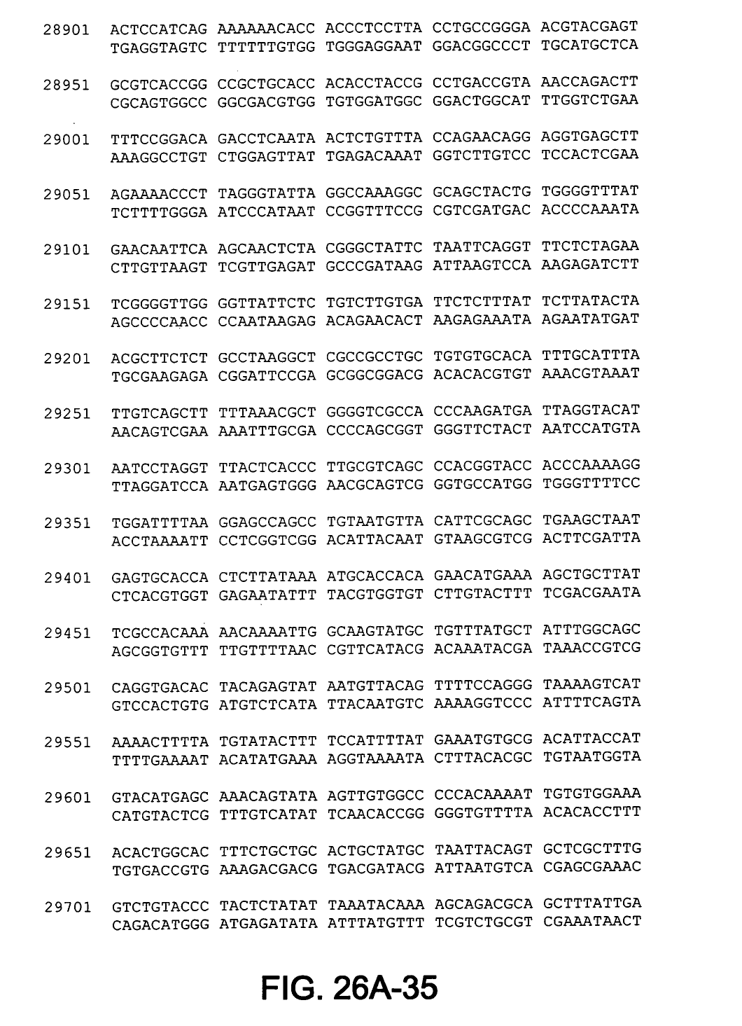

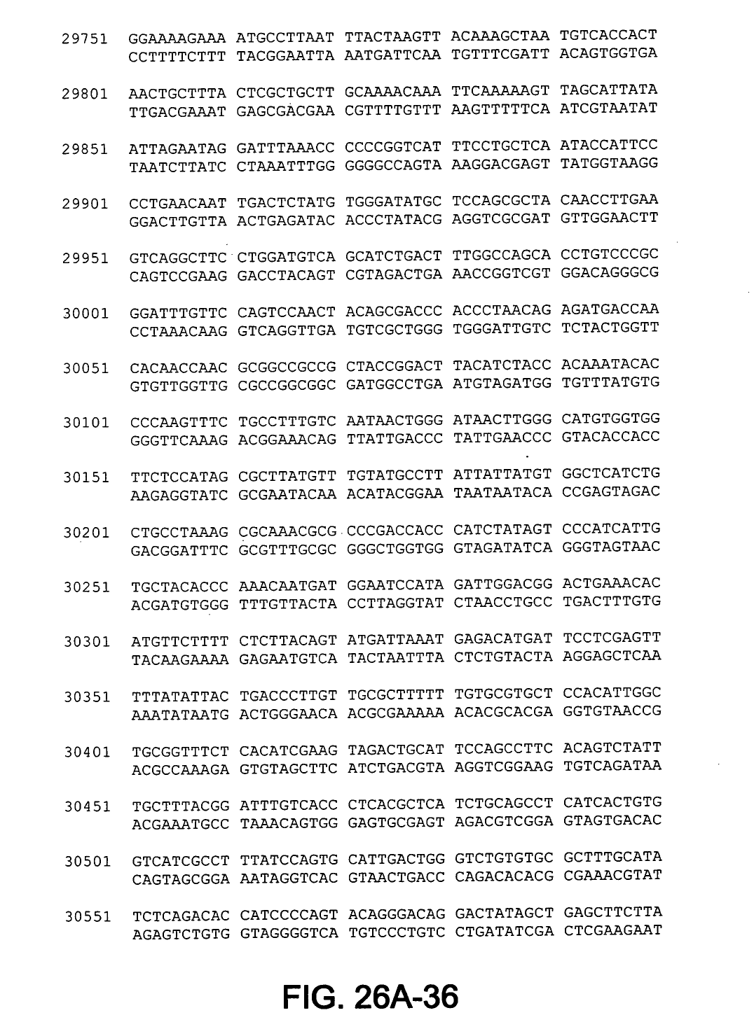

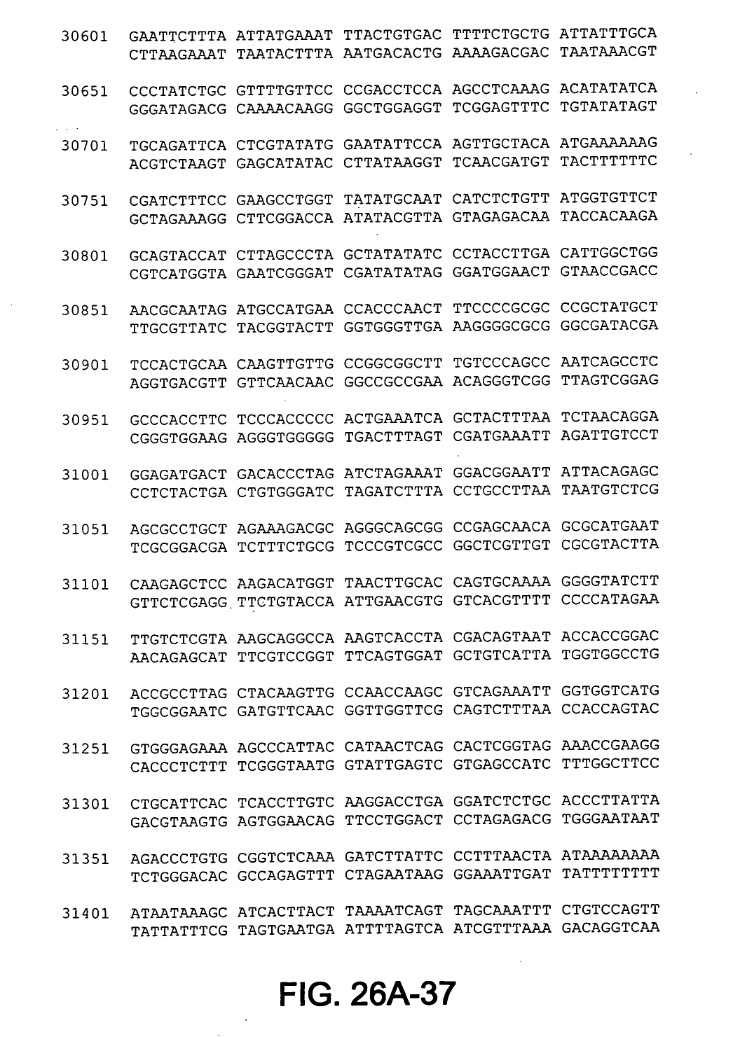

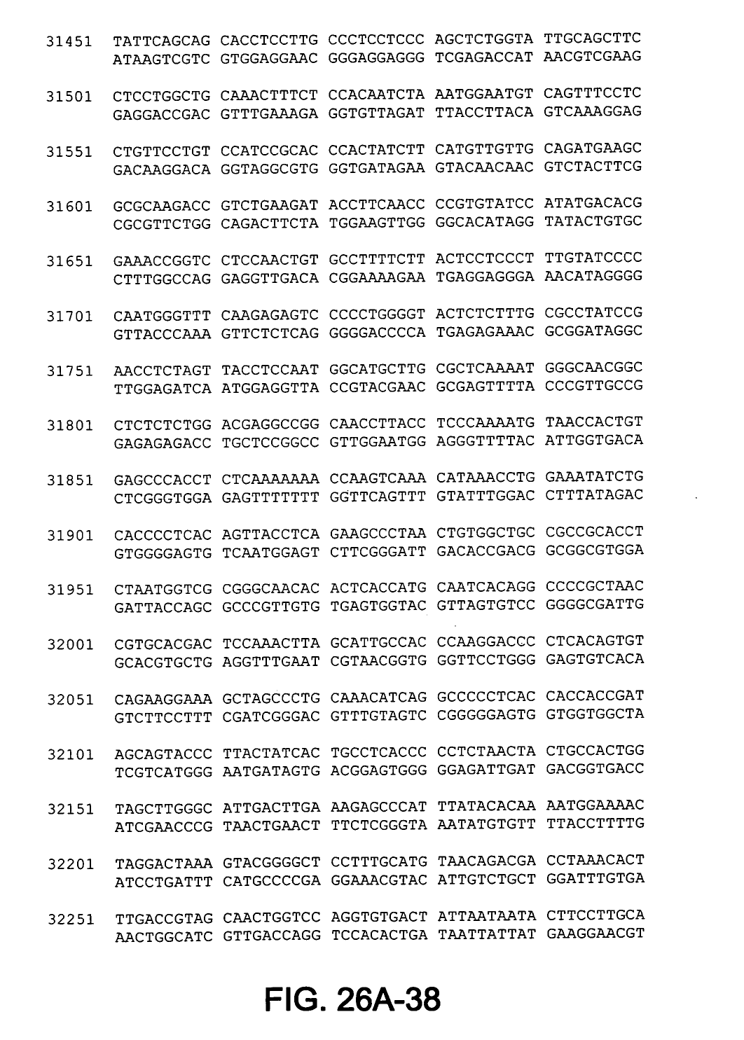

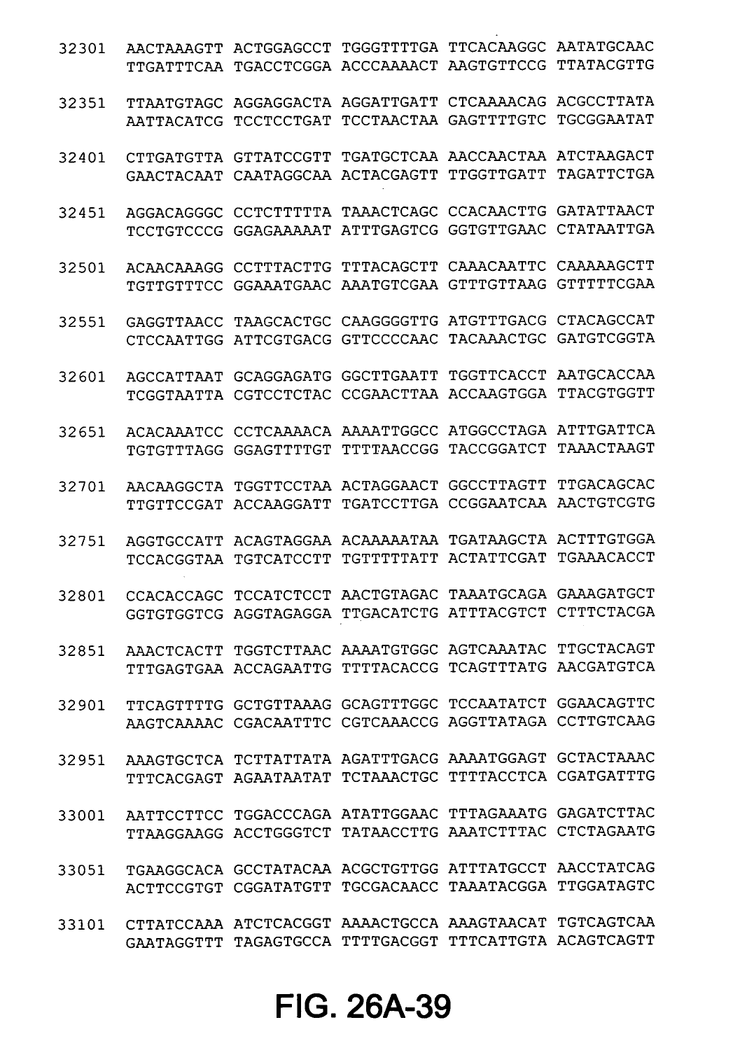

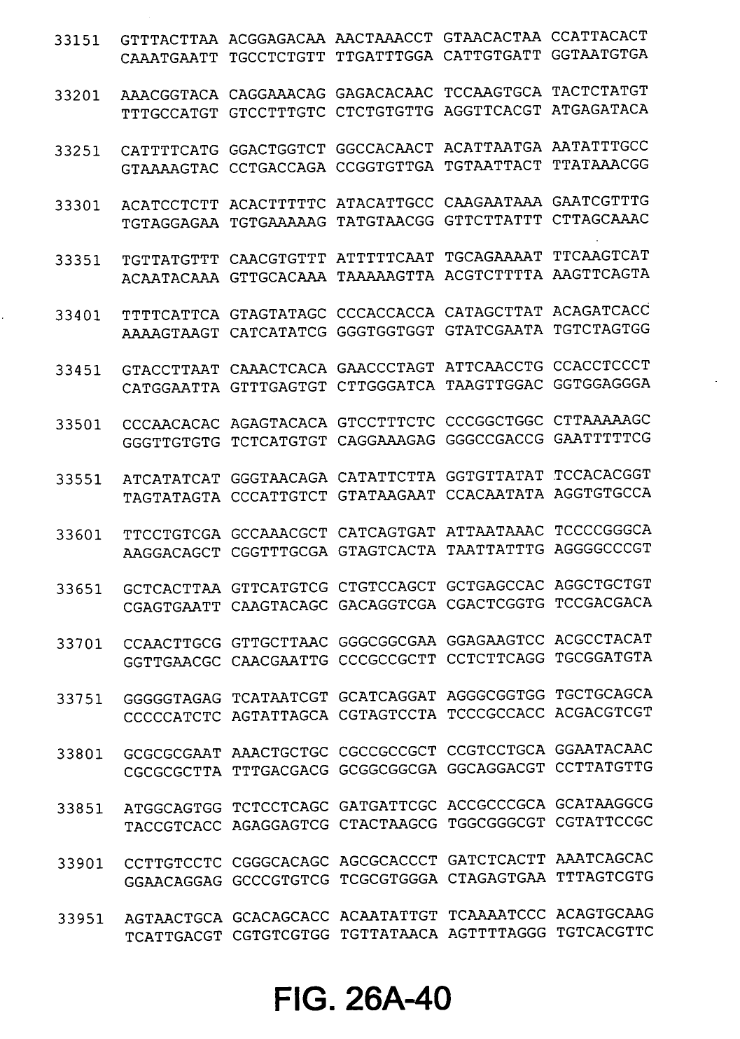

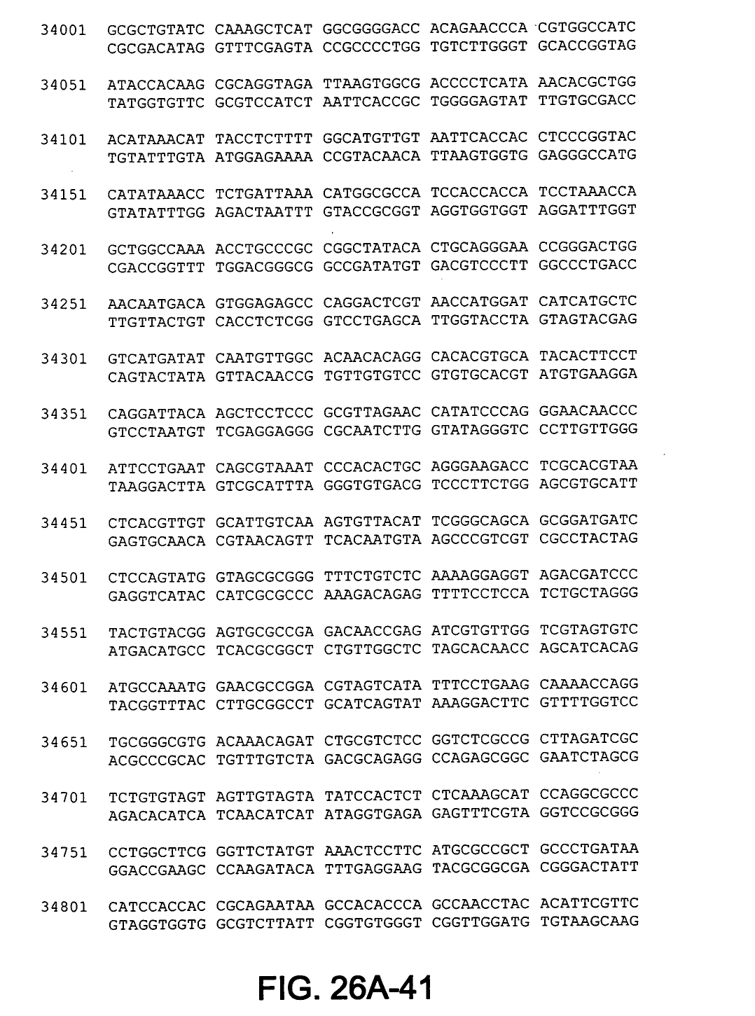

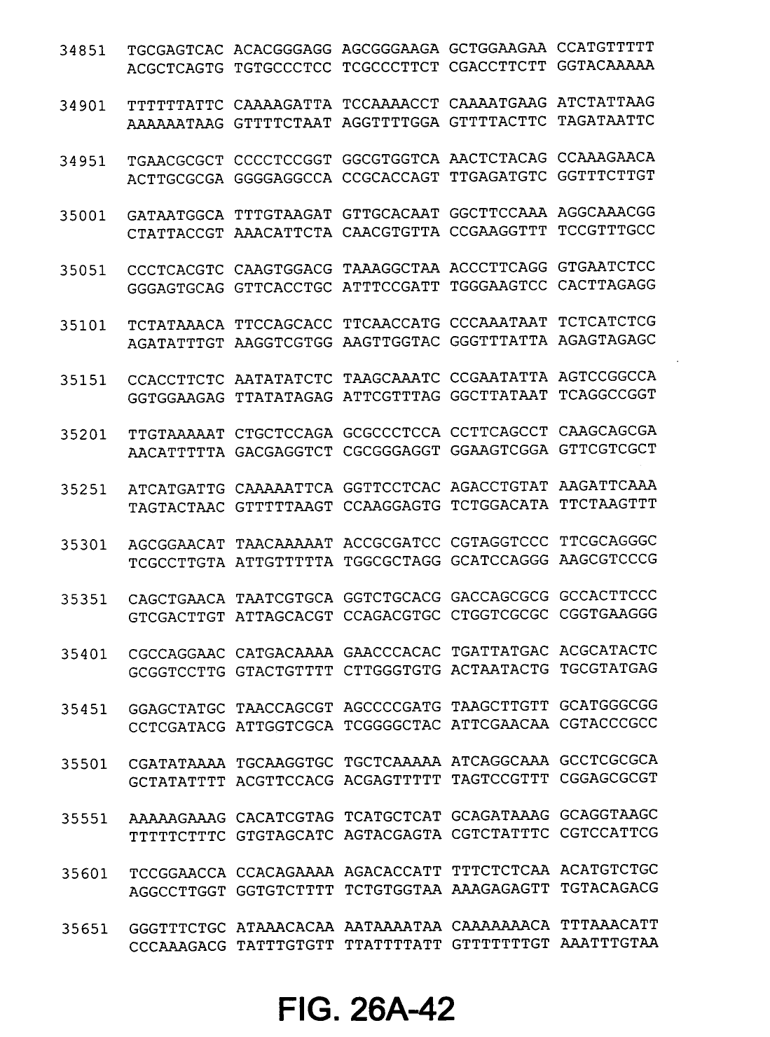

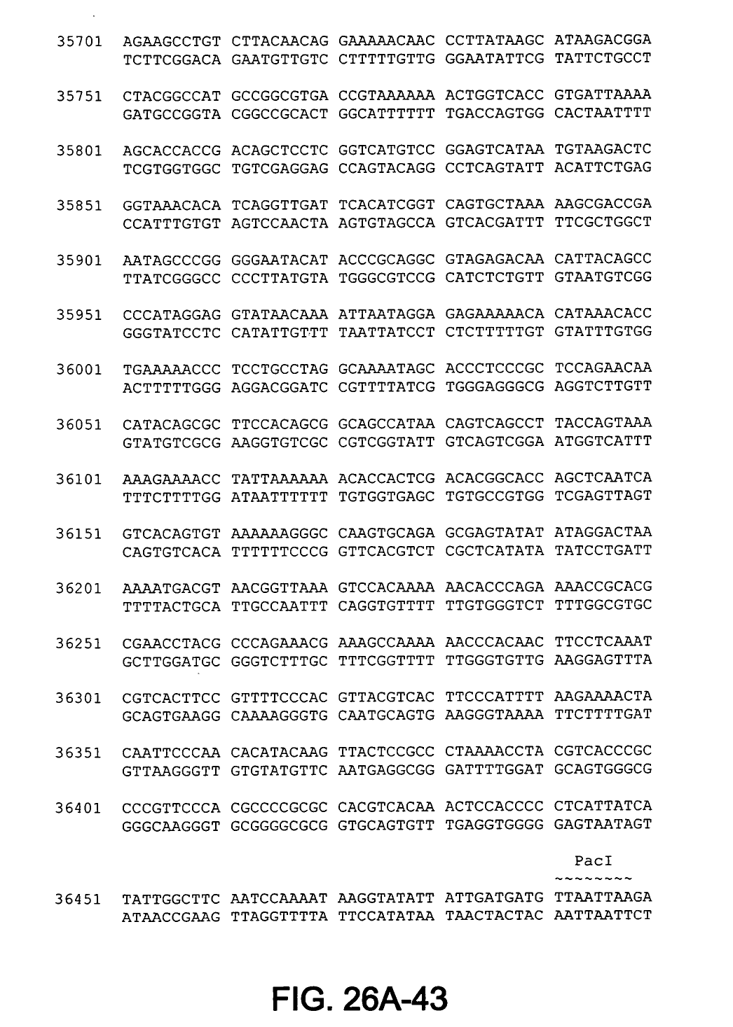

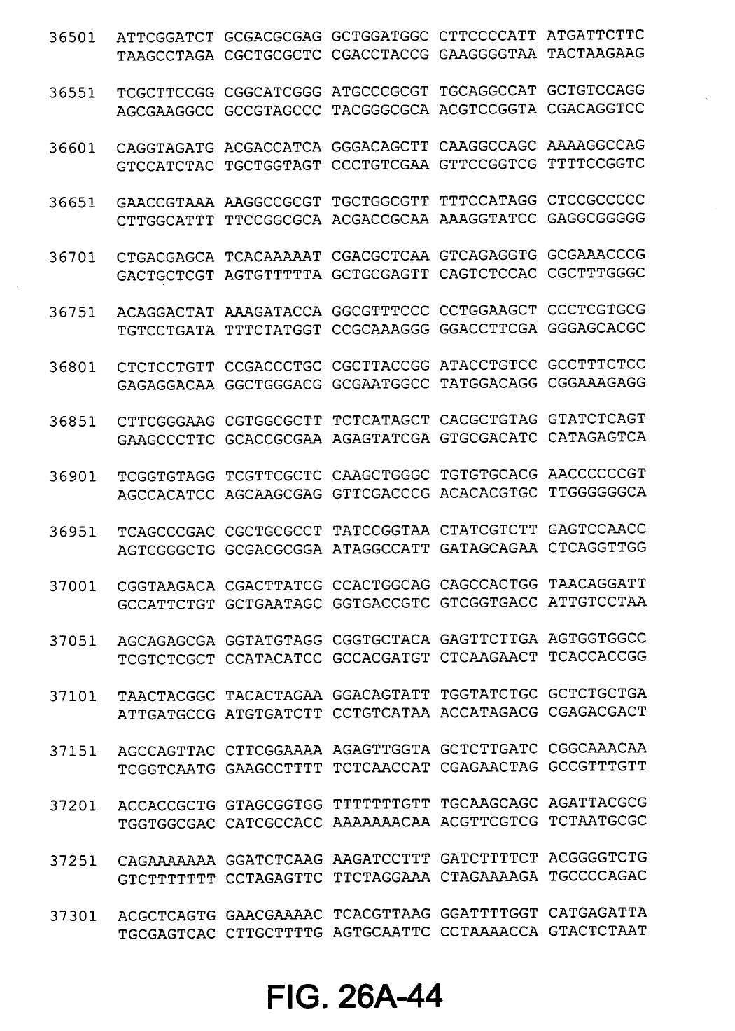

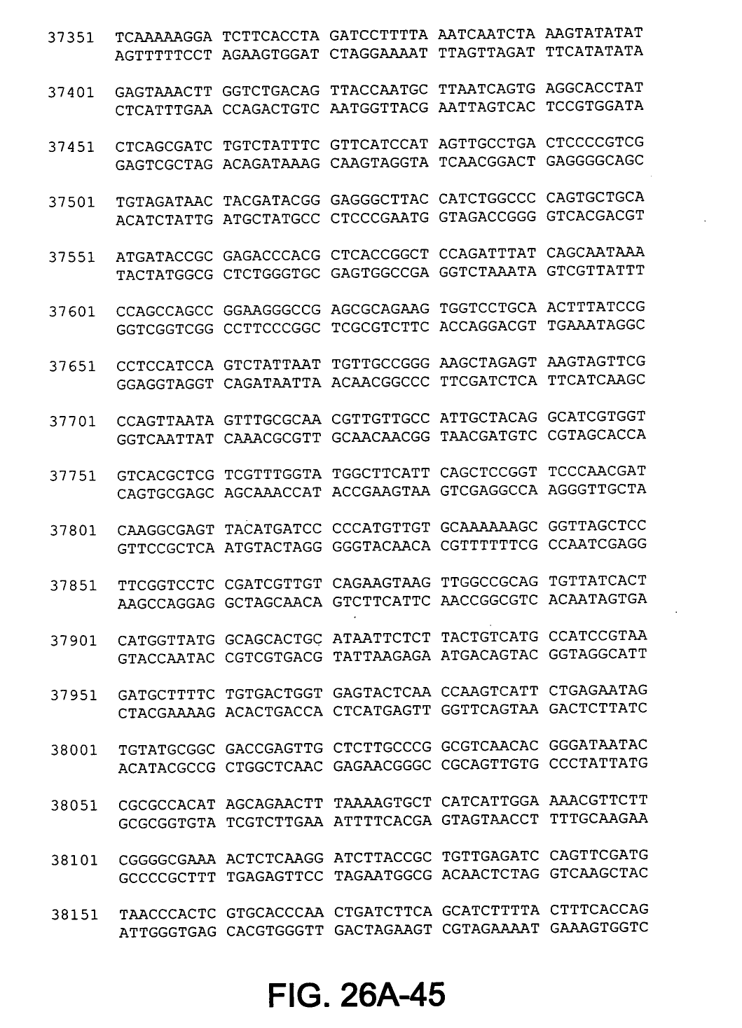

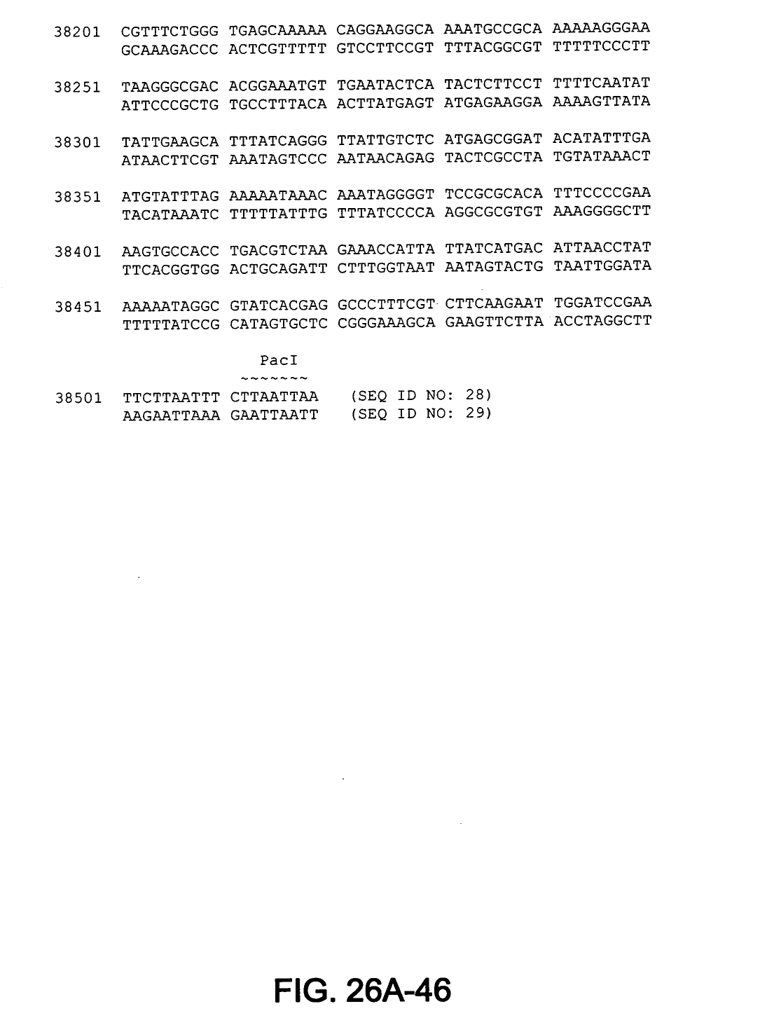

Construction of vector: shuttle plasmid and pre-adenovirus plasmid—Key steps performed in the construction of the vectors, including the pre-adenovirus plasmid denoted MRKAd5pol, is depicted in FIG. 22. Briefly, the adenoviral shuttle vector for the full-length inactivated HIV-1 pol gene is as follows. The vector MRKpdelE1(Pac/pIX/pack450)+CMVmin+BGHpA(str.) is a derivative of the shuttle vector used in the construction of the MRKAd5gag adenoviral pre-plasmid. The vector contains an expression cassette with the hCMV promoter (no intronA) and the bovine growth hormone polyadenylation signal. The expression unit has been inserted into the shuttle vector such that insertion of the gene of choice at a unique Bg/II site will ensure the direction of transcription of the transgene will be Ad5 E1 parallel when inserted into the MRKpAd5(E1−/E3+)Clal (or MRKpAdHVE3) pre-plasmid. The vector, similar to the original shuttle vector contains the Pac1 site, extension to the packaging signal region, and extension to the pIX gene. The synthetic full-length codon-optimized HIV-1 pol gene was isolated directly from the plasmid pV1Jns-HIV-pol-inact(opt). Digestion of this plasmid with Bgl II releases the pol gene intact (comprising a codon optimized IA pol sequence as disclosed in SEQ ID NO:3). The pol fragment was gel purified and ligated into the MRKpdelE1(Pac/pIX/pack450)+CMVmin+BGHpA(str.) shuttle vector at the Bg/II site. The clones were checked for the correct orientation of the gene by using restriction enzymes DraIII/Not1. A positive clone was isolated and named MRKpdel+hCMVmin+FL-pol+bGHpA(s). The genetic structure of this plasmid was verified by PCR, restriction enzyme and DNA sequencing. The pre-adenovirus plasmid was constructed as follows. Shuttle plasmid MRKpdel+hCMVmin+FL-pol+bGHpA(S) was digested with restriction enzymes Pac1 and Bst1107 I (or its isoschizomer, BstZ107 1) and then co-transformed into E. coli strain BJ5 183 with linearized (Cla1 digested) adenoviral backbone plasmid, MRKpAd(E1−/E3+)Cla1. The resulting pre-plasmid originally named MRKpAd+hCMvmin+FL-pol+bGHpA(S)E3+ is now referred to as “pMRKAd5pol”. The genetic structure of the resulting pMRKAd5pol was verified by PCR, restriction enzyme and DNA sequence analysis. The vectors were transformed into competent E. coli XL-1 Blue for preparative production. The recovered plasmid was verified by restriction enzyme digestion and DNA sequence analysis, and by expression of the pol transgene in transient transfection cell culture. The complete nucleotide sequence of this pMRKAd5HIV-1pol adenoviral vector is shown in FIGS. 26 A-1 to 26A-46.

Generation of research-grade recombinant adenovirus—The pre-adenovirus plasmid, pMRKAd5pol, was rescued as infectious virions in PER.C6® adherent monolayer cell culture. To rescue infectious virus, 12 μg of pMRKAd5pol was digested with restriction enzyme PacI (New England Biolabs) and 3.3 μg was transfected per 6 cm dish of PER.C6® cells using the calcium phosphate co-precipitation technique (Cell Phect Transfection Kit, Amersham Pharmacia Biotech Inc.). PacI digestion releases the viral genome from plasmid sequences allowing viral replication to occur after entry into PER.C6® cells. Infected cells and media were harvested 6-10 days post-transfection, after complete viral cytopathic effect (CPE) was observed. Infected cells and media were stored at ≦−60° C. This pol containing recombinant adenovirus is referred to herein as “MRKAd5pol”. This recombinant adenovirus expresses an inactivated HIV-1 Pol protein as shown in SEQ ID NO:6.

### EXAMPLE 20

### MRKAd5Nef Construction and Virus Rescue

Construction of vector: shuttle plasmid and pre-adenovirus plasmid—Key steps performed in the construction of the vectors, including the pre-adenovirus plasmid denoted MRKAd5nef, is depicted in FIG. 23. Briefly, as shown in Example 19 above, the vector MRKpdelE1(Pac/pIX/pack450)+CMVmin+BGHpA(str.) is the shuttle vector used in the construction of the MRKAd5gag adenoviral pre-plasmid. It has been modified to contain the Pac1 site, extension to the packaging signal region, and extension to the pIX gene. It contains an expression cassette with the hCMV promoter (no intronA) and the bovine growth hormone polyadenylation signal. The expression unit has been inserted into the shuttle vector such that insertion of the gene of choice at a unique Bgl11 site will ensure the direction of transcription of the transgene will be Ad5 E1 parallel when inserted into the MRKpAd5(E1−/E3+)Cla1 pre-plasmid. The synthetic full-length codon-optimized HIV-1 nef gene was isolated directly from the plasmid pV1Jns/nef (G2A,LLAA). Digestion of this plasmid with Bgl11 releases the pol gene intact, which comprises the nucleotide sequence as disclosed in SEQ ID NO:13. The nef fragment was gel purified and ligated into the MRKpdelE1+CMVmin+BGHpA(str.) shuttle vector at the Bgl11 site. The clones were checked for correction orientation of the gene by using restriction enzyme Sca1. A positive clone was isolated and named MRKpdelE1hCMVminFL-nefBGHpA(s). The genetic structure of this plasmid was verified by PCR, restriction enzyme and DNA sequencing. The pre-adenovirus plasmid was constructed as follows. Shuttle plasmid MRKpdelE1hCMVminFL-nefBGHpA(s) was digested with restriction enzymes Pac1 and Bst 1107 I (or its isoschizomer, BstZ107 I) and then co-transformed into E. coli strain BJ5183 with linearized (Cla1 digested) adenoviral backbone plasmid, MRKpAd(E1/E3+)Cla1. The resulting pre-plasmid originally named MRKpdelE1hCMVminFL-nefBGHpA(s) is now referred to as “pMRKAd5nef”. The genetic structure of the resulting pMRKAd5nef was verified by PCR, restriction enzyme and DNA sequence analysis. The vectors were transformed into competent E. coli XL-1 Blue for preparative production. The recovered plasmid was verified by restriction enzyme digestion and DNA sequence analysis, and by expression of the nef transgene in transient transfection cell culture. The complete nucleotide sequence of this pMRKAd5HIV-1nef adenoviral vector is shown in FIGS. 27A-1 to 27A-44.

Generation of research-grade recombinant adenovirus—The pre-adenovirus plasmid, pMRKAd5nef, was rescued as infectious virions in PER.C6® adherent monolayer cell culture. To rescue infectious virus, 12 μg of pMRKAdnef was digested with restriction enzyme Pac1 (New England Biolabs) and 3.3 μg was transfected per 6 cm dish of PER.C6® cells using the calcium phosphate co-precipitation technique (Cell Phect Transfection Kit, Amersham Pharmacia Biotech Inc.). Pac1 digestion releases the viral genome from plasmid sequences allowing viral replication to occur after entry into PER.C6® cells. Infected cells and media were harvested 6-10 days post-transfection, after complete viral cytopathic effect (CPE) was observed. Infected cells and media were stored at ≦−60° C. This nef containing recombinant adenovirus is now referred to as “MRKAd5nef”.

### EXAMPLE 21

### Construction of Murine CMV Promoter Containing Shuttle Vectors for Inactivated Pol and Nef/G2A,LLAA

The murine CMV (mCMV) was amplified from the plasmid pMH4 (supplied by Frank Graham, McMaster University) using the primer set: mCMV (Not I) Forward: 5′-ATA AGA ATG CGG CCG CCA TAT ACT GAG TCA TFA GG-3′(SEQ ID NO: 20); mCMV (Bgl II)Reverse: 5′-AAG GAA GAT CTA CCG ACG CTG GTC GCG CCT C-3′(SEQ ID NO:21). The underlined nucleotides represent the Not I and the Bgl II sites respectively for each primer. This PCR amplicon was used for the construction of the mCMV shuttle vector containing the transgene in the E1 parallel orientation. The hCMV promoter was removed from the original shuttle vector (containing the hCMV-gag-bGHpA transgene in the E1 parallel orientation) by digestion with Not I and Bgl II. The mCMV promoter (Not I/Bgl II digested PCR product) was inserted into the shuttle vector in a directional manner. The shuttle vector was then digested with Bgl II and the gag reporter gene (Bgl II fragment) was re-inserted back into the shuttle vector. Several clones were screened for correct orientation of the reporter gene. For the construction of the mCMV-gag in the El antiparallel orientation, the mCMV promoter was amplified from the plasmid pMH4 using the following primer set: mCMV (Asc I) Forward: 5′-ATA AGA ATG GCG CGC CAT ATA CTG AGT CAT TAG G (SEQ ID NO:22); mCMV (Bgl II) Reverse: 5′ AAG GAA GAT CTA CCG ACG CTG GTC GCG CCT C (SEQ ID NO:21). The underlined nucleotides represent the Asc I and Bgl II sites, respectively for each primer. The shuttle vector containing the hCMV-gag transgene in the E1 antiparallel orientation was digested with Asc1 and Bgl11 to remove the hCMV-gag portion of the transgene. The mCMV promoter (Ascl/Bgl11 digested PCR product) was inserted into the shuttle vector in a directional manner. The vector was then digested with Bgl11 and the gag reporter gene (Bgl11 fragment) was re-inserted. Several clones were screened for correct orientation of the reporter gene. For each of the full length IA pol and full length nef/G2A,LLAA genes, cloning was performed using the unique Bgl II site within the mCMV-bGHpA shuttle vector. The pol and nef genes were excised from their respective pV1Jns plasmids by Bgl II digestion.

### EXAMPLE 22

### Construction of mCMV Full Length Inactivated Pol and Full Length nef/G2A.LLAA Adenovectors

Each of these transgenes of Example 21 were inserted into the modified shuttle vector in both the E1 parallel and E1 anti-parallel orientations. Pac1 and BstZ1101I digestion of each shuttle vector was performed and each specific transgene fragment containing the flanking Ad5 sequences was isolated and co-transformed with Cla I digested MRKpAd5(E3+) or MRKpAd5(E3−) adenovector plasmids via bacterial homologous recombination in BJ5183 E. coli cells. Recombinant pre-plasmid adenovectors containing the various transgenes in both the E3− and E3+ versions (and in the E1 parallel and E1 antiparallel orientations) were subsequently prepared in large scale following transformation into XL-1 Blue E. coli cells and analyzed by restriction analysis and sequencing.

### EXAMPLE 23

### Construction of hCMV-tpa-nef (LLAA) Adenovector

The tpa-nef gene was amplified out from GMP grade pV1Jns-tpanef (LLAA) vector using the primer sets: Tpanef (BamHI) F 5′-ATT GGA TCC ATG GAT GCA ATG AAG AGA GGG (SEQ ID NO:23); Tpanef (BamlHI) R 5′-ATA GGA TCC TTA GCA GTC CTT GTA GTA CTC G (SEQ ID NO:24). The resulting PCR product was digested with BamHI, gel purified and cloned into the Bgl II site of MRKAd5CMV-bGHpA shuttle vector (Bgl II digested and calf intestinal phosphatase treated). Clones containing the tpanef (LLAA) gene (see SEQ ID NO:15 for complete coding region) in the correct orientation with respect to the hCMV promoter were selected following Sca I digestion. The resulting MRKAd5tpanef shuttle vector was digested with Pac I and Bst Z1101 and cloned into the E3+MRKAds adenovector via bacterial homologous recombination techniques.

### EXAMPLE 24

### Immunogenicity of MRKAd5pol and MRKAd5nef Vaccine

Materials and Methods—Rodent Immunization—Groups of N=10 BALB/c mice were immunized i.m. with the following vectors: (1) MRKAd5hCMV-IApol (E3+) at either 10ˆ7vp and 10ˆ9vp; and (2) MRKAd5hCMV-IApol (E3−) at either 10ˆ7vp and 10ˆ9 vp. At 7 weeks post dose, 5 of the 10 mice per cohort were boosted with the same vector and dose they initially received. At 3 weeks post the second does, sera and spleens were collected from all the animals for RT ELISA and IFNg ELIspot analyses, respectively. For all rodent immunizations, the Ads vectors were diluted in 5 mM Tris, 5% sucrose, 75 mM NaCl, 1 mM MgCl2, 0.005% polysorbate 80, pH 8.0. The total dose was injected to both quadricep muscles in 50 μL aliquots using a 0.3-mL insulin syringe with 28½ G needles (Becton-Dickinson, Franklin Lakes, N.J.).

Groups of N=10 C57/BL6 mice were immunized i.m. with the following vectors: (1) MRKAd5hCMV-nef(G2A,LLAA) (E3+) at either 10ˆ7vp and 10ˆ9vp; (2) MRKAd5mCMV-nef(G2A,LLAA) (E3+) at either 10ˆ7vp and 10ˆ9vp; and (3) MRKAd5mCMV-tpanef(LLAA) (E3+) at either 10ˆ7vp and 10ˆ9vp. At 7 weeks post dose, 5 of the 10 mice per cohort were boosted with the same vector and dose they initially received. At 3 weeks post the second does, sera and spleens were collected from all the animals for RT ELISA and INg ELIspot analyses, respectively.

Non-human Primate immunization—Cohorts of 3 rhesus macaques (2-3 kg) were vaccinated with the following Ad vectors: (1) MRKAd5hCMV-IApol (E3+) at either 10ˆ9vp and 10ˆ1vp dose; and (2) MRKAd5hCMV-IApol (E3−) at either 10ˆ9vp and 10ˆ11vp; (3) MRKAd5hCMV-nef(G2A,LLAA) (E3+) at either 10ˆ9vp and 10ˆ11vp; and (4) MRKAd5mCMV-nef(G2A,LLAA) (E3+) at either 10ˆ9vp and 10ˆ11vp. The vaccine was administered to chemically restrained monkeys (10 mg/kg ketamine) by needle injection of two 0.5 mL aliquots of the Ad vectors (in 5 mM Tris, 5% sucrose, 75 mM NaCl, 1 mM MgCl2, 0.005% polysorbate 80, pH 8.0) into both deltoid muscles. The animals were immunized twice at a 4 week interval (T=0, 4 weeks).

Murine anti-RT and anti-nef ELISA—Anti-RT titers were obtained following standard secondary antibody-based ELISA. Maxisorp plates (NUNC, Rochester, N.Y.) were coated by overnight incubation with 100 μL of 1 μg /mL HIV-1 RT protein (Advanced Biotechnologies, Columbia, Md.) in PBS. For anti-nef ELISA, 100 uL of 1 ug/mL HIV-1 nef (Advanced Biotechnologies, Columbia, Md.) was used to coat the plates. The plates were washed with PBS/0.05% Tween 20 using Titertek MAP instrument (Hunstville, Ala.) and incubated for 2 h with 200 μL/well of blocking solution (PBS/0.05% tween/1% BSA). An initial serum dilution of 100-fold was performed followed by 4-fold serial dilution. 100-μL aliquots of serially diluted samples were added per well and incubated for 2 h at room temperature. The plates were washed and 100 μL of 1/1000-diluted HRP-rabbit anti-mouse IgG (ZYMED, San Francisco, Calif.) were added with 1 h incubation. The plates were washed thoroughly and soaked with 100 μL 1,2-phenylenediamine dihydrochloride/hydrogen peroxide (DAKO, Norway) solution for 15 min. The reaction was quenched by adding 100 μL of 0.5M H₂SO4 per well. OD₄₉₂ readings were recorded using Titertek Multiskan MCC/340 with S20 stacker. Endpoint titers were defined as the highest serum dilution that resulted in an absorbance value of greater than or equal to 0.1 OD₄₉₂ (2.5 times the background value).

Non-human primate and murine ELIspot assays—The enzyme-linked immuno-spot (ELISpot) assay was utilized to enumerate antigen-specific INFγ-secreting cells from mouse spleens (Miyahira, et al. 1995, J. Immunol. Methods 181:45-54) or macaque PBMCs. Mouse spleens were pooled from 5 mice/cohort and single cell suspensions were prepared at 5×10⁶/mL in complete RPMI media (RPMI1640, 10% FBS, 2 mM L-glutamine, 100 U/mL Penicillin, 100 u/mL streptomycin, 10 mM Hepes, 50 uM β-ME). Rhesus PBMCs were prepared from 8-15 mL of heparinized blood following standard Ficoll gradient separation (Coligan, et al, 1998, Current Protocols in Immunology. John Wiley & Sons, Inc.). Multiscreen opaque plates (Millipore, France) were coated with 100 μL/well of either 5 μg/mL purified rat anti-mouse IFN-γ IgG1, clone R4-6A2 (Pharmingen, San Diego, Calif.), or 15 ug/mL mouse anti-human IFN-γ IgG₂ₐ (Cat. No. 1598-00, R&D Systems, Minneapolis, Minn.) in PBS at 4° C. overnight for murine or monkey assays, respectively. The plates were washed with PBS/penicillin/streptomycin and blocked with 200 μL/well of complete RPMI media for 37° C. for at least 2 h.

To each well, 50 μL of cell samples (4-5×10⁵ cells per well) and 50 μL of the antigen solution were added. To the control well, 50 μL of the media containing DMSO were added; for specific responses, either selected peptides or peptide pools (4 ug/mL per peptide final concentration) were added. For BALB/c mice immunized with the pol constructs, stimulation was conducted using a pool of CD4⁺-epitope containing 20-mer peptides (aa21-40, aa411-430, aa641-660, aa731-750, aa771-790) or a pool of CD8⁺-epitope containing peptides (aa201-220, aa311-330, aa781-800). For C57/BL6 mice immunized with the nef construct, either aa51-70 (CD8⁺ T cell epitope) or aa81 -100 (CD4⁺) peptide derived from nef sequence was added for specific stimulation. In monkeys, the responses against pol were evaluated using two pools (L and R) of 20-aa peptides that encompass the entire pol sequence and overlap by 10 amino acids. In monkeys vaccinated with the nef constructs, a single pool containing 20-mer peptides covering the entire HIV-1 nef sequence and overlapping by 10 aa was used. Each sample/antigen mixture was performed in triplicate wells for murine samples or in duplicate wells for rhesus PBMCs. Plates were incubated at 37° C., 5% CO₂, 90% humidity for 20-24 h. The plates were washed with PBS/0.05% Tween 20 and incubated with 100 μL/well of either 1.25 μg/mL biotin-conjugated rat anti-mouse IFN-γ mAb, clone XMG1.2 (Pharmingen) or of 0.1 ug/mL biotinylated anti-human IFN-gamma goat polyclonal antibody (R&D Systems) at 4° C. overnight. The plates were washed and incubated with 100 μL/well 1/2500 dilution of strepavidin-alkaline phosphatase conjugate (Pharmingen) in PBS/0.005% Tween/5% FBS for 30 min at 37° C. Spots were developed by incubating with 100 μL/well 1-step NBT/BCIP (Pierce Chemicals) for 6-10 min. The plates were washed with water and allowed to air dry. The number of spots in each well was determined using a dissecting microscope and the data normalized to 10⁶ cell input.

Non-human Primate anti-RTELISA—The pol-specific antibodies in the monkeys were measured in a competitive RT EIA assay, wherein sample activity is determined by the ability to block RT antigen from binding to coating antibody on the plate well. Briefly, Maxisorp plates were coated with saturating amounts of pol positive human serum (#97111234). 250 uL of each sample is incubated with 15 uL of 266 ng/mL RT recombinant protein (in RCM 563, 1% BSA, 0.1% tween, 0.1% NaN₃) and 20 uL of lysis buffer (Coulter p24 antigen assay kit) for 15 min at room temperature. Similar mixtures are prepared using serially diluted samples of a standard and a negative control which defines maximum RT binding. 200 uL/well of each sample and standard were added to the washed plate and the plate incubated 16-24 h at room temperature. Bound RT is quantified following the procedures described in Coulter p24 assay kit and reported in milliMerck units per mL arbitrarily defined by the chosen standard.

Results—Rodent Studies—BALB/c mice (n=5 mice/cohort) were immunized once or twice with varying doses of MRKAd5hCMV-IApol(E3+) and MRKAd5hCMV-IApol(E3−). At 3 weeks after the second dose, Anti-pol IgG levels were determined by an ELISA assay using RT as a surrogate antigen. Cellular response were quantified via IFNγ ELISpot assay against pools of pol-epitope containing peptides. The results of these assays are summarized in Table 10. The results indicate that the mouse vaccinees exhibited detectable anti-RT IgGs with an adenovector dose as low as 10ˆ7vp. The humoral responses are highly dose-dependent and are boostable with a second immunization. One or two doses of either pol vectors elicit high frequencies of antigen-specific CD4⁺ and CD8⁺ T cells; the responses are weakly dose-dependent but are boostable with a second immunization.

TABLE 10
Immunogenicity of MRKAd5pol Vectors in BALB/c mice.

| | | | | | | | SFC/10\hat{ }6 cells^{c} | | |
|---|---|---|---|---|---|---|---|---|---|
| | | | | | | | | CD4+ | CD8+ |
| | | | No. of | Anti-RT IgG Titers^{a} | | | | peptide | peptide |
| Group | Vaccine | Dose | Doses | GMT | +SE | −SE | Medium | pool | pool |
| 1 | MRKAd5hCMVFLpol (E3+) | 10\hat{ }7 vp | 2 | 310419 | 301785 | 153020 | 1(1) | 75(4) | 2313(67) |
| | | | 1 | 919 | 372 | 265 | 1(1) | 72(9) | 533(41) |
| 2 | MRKAd5hCMVFLpol (E3+) | 10\hat{ }9 vp | 2 | 1638400^{b} | 0 | 0 | 2(2) | 114(9) | 2063(182) |
| | | | 1 | 713155 | 528520 | 303555 | 1(1) | 48(7) | 733(89) |
| 3 | MRKAd5hCMVFLpol (E3−) | 10\hat{ }7 vp | 2 | 310419 | 386218 | 172097 | 0(0) | 223(7) | 2607(27) |
| | | | 1 | 6400 | 14013 | 4393 | 10(8) | 141(21) | 409(28) |
| 4 | MRKAd5hCMVFLpol (E3−) | 10\hat{ }9 vp | 2 | 1638400^{b} | 0 | 0 | 1(1) | 160(13) | 2385(11) |
| | | | 1 | 1241675^{b} | 396725 | 300661 | 0(0) | 39(13) | 833(63) |
| 5 | Naïve | none | none | 57 | 9 | 7 | 9(2) | 11(4) | 10(1) |

^{a}GMT, geometric mean titer of the cohort of 5 mice; SE, standard error of the gemetric mean
^{b}Near or at the upper limit of the serial dilution; hence, could be greater than this value
^{c}No. of Spot-forming Cells per million splecnoytes; mean values of triplicates are reported along with standard errors in parenthesis.

C57/BL6 mice were immunized once or twice with varying doses of MRKAd5hCMV-nef(G2A,LLAA) (E3+), MRKAd5mCMV-nef(G2A,LLAA)(E3+) at either 10ˆ7vp and(3) MRKAd5mCMV-tpanef(LLAA) (E3+) at either 10ˆ7 vp and 10ˆ9 vp. The immune response were analyzed using similar protocols and the results are listed in Table 11. While anti-nef IgG responses could not be detected in this model system with any of the constructs, there are strong indications of a cellular immunity generated against nef using the ELIspot assay.

TABLE 11
Immunogenicity of MRKAd5nef Vectors in C57/BL6 mice.

| | | | | | | | SFC/10\hat{ }6 cells^{b} | | |
|---|---|---|---|---|---|---|---|---|---|
| | | | No. of | Anti-nef IgG Titers^{a} | | | | aa51-70 | aa81-100 |
| Group | Vaccine | Dose | Doses | GMT | +SE | −SE | Medium | CD8+ | CD4+ |
| 1 | MRKAd5hCMVFLnef (E3+) | 10\hat{ }7 vp | 2 | 174 | 70 | 50 | 1(1) | 23(1) | 1(1) |
| | | | 1 | 132 | 42 | 32 | 0(0) | 0(0) | 0(0) |
| 2 | MRKAd5hCMVFLnef (E3+) | 10\hat{ }9 vp | 2 | 174 | 70 | 50 | 0(0) | 61(7) | 4(2) |
| | | | 1 | 132 | 42 | 32 | 1(1) | 62(7) | 3(1) |
| 3 | MRKAd5mCMVFLnef (E3+) | 10\hat{ }7 vp | 2 | 132 | 42 | 32 | 3(1) | 15(5) | 5(2) |
| | | | 1 | 115 | 46 | 33 | 3(2) | 3(2) | 4(2) |
| 4 | MRKAd5mCMVFLnef (E3+) | 10\hat{ }9 vp | 2 | 132 | 42 | 32 | 4(2) | 83(13) | 5(1) |
| | | | 1 | 132 | 42 | 32 | 2(1) | 29(2) | 4(0) |
| 5 | MRKAd5mCMVtpanef(E3+) | 10\hat{ }7 vp | 2 | 132 | 42 | 32 | 3(2) | 14(2) | 5(1) |
| | | | 1 | 100 | 0 | 0 | 3(1) | 13(4) | 10(3) |
| 6 | MRKAd5mCMVtpanef(E3+) | 10\hat{ }9 vp | 2 | 230 | 170 | 98 | 3(2) | 145(29) | 4(0) |
| | | | 1 | 115 | 46 | 33 | 7(1) | 151(14) | 10(0) |
| 7 | Naïve | none | none | 152 | 78 | 52 | 21(2) | 18(6) | 26(3) |

^{a}GMT, geometric mean titer of the cohort of 5 mice; SE, standard error of the gemetric mean
^{b}No. of spot-forming cells per million splecnoytes; mean values of triplicates are reported along with standard errors in parenthesis.

Monkey Studies—Cohorts of 3 rhesus macaques were immunized with 2 doses of MRKAd5hCMV-IApol(E3+) and MRKAd5hCMV-IApol(E3−). The number of antigen-specific T cells (per million PBMCs) were enumerated using one of two peptide pools (L and R) that cover the entire pol sequence; the results are listed in Table 12. Moderate-to-strong T cell responses were detected in the vaccines using either constructs even at a low dose of 10ˆ9vp. Longitudinal analyses of the anti-RT antibody titers in the animals suggest that the pol transgene product is expressed efficiently to elicit a humoral response (Table 13). It would appear that generally higher immune responses were observed in animals that received the E3− construct compared to the E3+ virus.

TABLE 12
Pol-specific T Cell Responses in MRKAd5pol Immunized Rhesus Macaques.

| | | Prebleed | | | T = 4 | | | T = 7 | | | T = 16 | | |
|---|---|---|---|---|---|---|---|---|---|---|---|---|---|
| Vaccine (T = 0, 4 wks) | Monk # | Mock | Pol L | Pol R | Mock | Pol L | Pol R | Mock | Pol L | Pol R | Mock | Pol L | Pol R |
| MRKAd5hCMV-lApol(E3+) | 99C100 | 1 | 0 | 0 | 1 | 38 | 31 | 0 | 52 | 146 | 0 | 49 | 715 |
| 10\hat{ }11 vp | 99C215 | 1 | 2 | 2 | 10 | 98 | 249 | 1 | 109 | 305 | 22 | 88 | 250 |
| | 99D201 | 5 | 5 | 4 | 6 | 149 | 95 | 0 | 40 | 35 | 0 | 35 | 18 |
| MRKAd5hCMV-lApol(E3+) | 99D212 | 0 | 2 | 0 | 4 | 331 | 114 | 0 | 58 | 14 | 0 | 6 | 6 |
| 10\hat{ }9 vp | 99D180 | 0 | 4 | 2 | 0 | 19 | 192 | 4 | 36 | 156 | 5 | 38 | 106 |
| | 99C201 | 8 | 5 | 21 | 6 | 62 | 62 | 0 | 18 | 32 | 1 | 14 | 65 |
| MRKAd5hCMV-lApol(E3−) | 99D239 | 5 | 2 | 2 | 20 | 82 | 172 | 1 | 66 | 114 | 9 | 21 | 40 |
| 10\hat{ }11 vp | 99C186 | 4 | 12 | 6 | 5 | 120 | 421 | 2 | 271 | 489 | 16 | 875 | 530 |
| | 99C084 | 1 | 8 | 9 | 8 | 84 | 464 | 0 | 14 | 236 | 1 | 24 | 264 |
| MRKAd5hCMV-lApol(E3−) | CC7C | 10 | 10 | 8 | 12 | 724 | 745 | 4 | 322 | 376 | 4 | 188 | 176 |
| 10\hat{ }9 vp | CD1G | 2 | 0 | 1 | 5 | 474 | 468 | 0 | 232 | 212 | 0 | 101 | 121 |
| | CD11 | 6 | 6 | 12 | 10 | 98 | 110 | 5 | 60 | 80 | 8 | 25 | 34 |
| Naïve | 083Q | nd | nd | nd | nd | nd | nd | 4 | 2 | 2 | 2 | 1 | 2 |

nd, not determined
Reported are SFC per million PBMCs; mean of duplicate wells.

TABLE 13
Anti-RT Ig Levels in MRKAd5pol Immunized macaques.
RT ANTIBODY ASSAY TITERS IN mMU/mL

| Vaccine/Monkey Tag | T = 4 | T = 7 | T = 12 | T = 16 |
|---|---|---|---|---|
| MRKAd5hCMV-IApol(E3+), 10\hat{ }11 vp | | | | |
| 99C100 | 61 | 1999 | 5928 | 4768 |
| 99C215 | 81 | 1541 | 2356 | 2767 |
| 99D201 | 53 | 336 | 539 | 387 |
| MRKAd5hCMV-IApol(E3+), 10\hat{ }9 vp | | | | |
| 99D212 | 10 | 40 | 49 | 68 |
| 99D180 | <10 | 36 | 79 | 93 |
| 99C201 | <10 | 37 | 71 | 76 |
| MRKAd5hCMV-IApol(E3−), 10\hat{ }11 vp | | | | |
| 99D239 | 44 | 460 | 1234 | 1015 |
| 99C186 | 21 | 233 | 480 | 345 |
| 99C084 | 235 | 2637 | 2858 | 1626 |
| MRKAd5hCMV-IApol(E3−), 10\hat{ }9 vp | | | | |
| CC7C | 32 | 175 | 306 | 235 |
| CD1G | 20 | 140 | 273 | 419 |
| CD11 | 15 | 112 | 149 | 237 |

When rhesus macaques were immunized i.m. with two doses of MRKAd5nef constructs, vigorous T cell responses ranging from 100 to as high as 1100 per million were observed in 8 of 12 vaccinees (Table 14). The efficacies of the mCMV- and hCMV-driven nef constructs are comparable on the basis of the data generated thus far.

TABLE 14
Nef-specific T cell Responses in MRKAd5nef Immunized Rhesus Macaques.

| | | Pre | | T = 4 | | T = 7 | | T = 16 | |
|---|---|---|---|---|---|---|---|---|---|
| Vaccine (T = 0, 4 wks) | Monk # | Mock | Nef | Mock | Nef | Mock | Nef | Mock | Nef |
| MRKAd5hCMV-nef(G2A, LLAA) (E3+) | CD2D | 0 | 4 | 31 | 440 | 4 | 368 | 1 | 251 |
| 10\hat{ }11 vp | CC7B | 0 | 0 | 2 | 521 | 0 | 178 | 1 | 1522 |
| | CC61 | 2 | 9 | 31 | 112 | 0 | 108 | 11 | 100 |
| MRKAd5hCMV-nef(G2A, LLAA) (E3+) | CC2K | 9 | 9 | 6 | 52 | 0 | 35 | 0 | 15 |
| 10\hat{ }9 vp | CD15 | 5 | 4 | 30 | 998 | 2 | 586 | 0 | 434 |
| | CD16 | 6 | 1 | 6 | 1146 | 0 | 369 | 1 | 212 |
| MRKAd5mCMV-nef(G2A, LLAA) (E3+) | 99D191 | 1 | 5 | 4 | 614 | 0 | 298 | 2 | 419 |
| 10\hat{ }11 vp | 99D144 | 4 | 6 | 5 | 434 | 0 | 1100 | 2 | 932 |
| | 99C193 | 1 | 2 | 1 | 58 | 1 | 22 | 0 | 64 |
| MRKAd5mCMV-nef(G2A, LLAA) (E3+) | 99D224 | 1 | 11 | 14 | 231 | 1 | 125 | 0 | 70 |
| 10\hat{ }9 vp | 99D250 | 8 | 9 | 4 | 108 | 0 | 54 | 0 | 5 |
| | 99C120 | 1 | 6 | 20 | 299 | 0 | 92 | 0 | 79 |
| Naïve | 083Q | nd | nd | 18 | 22 | 4 | 5 | 2 | 1 |

### EXAMPLE 25

### Comparison of Clade B vs. Clade C T Cell Responses in HIV-Infected Subjects

PBMC samples collected from two dozens of patients infected with HIV-1 in US were tested in ELISPOT assays with peptide pools of 20-mer peptides overlapping by 10 amino acids. Four different peptide pools were tested for cross-clade recognition, and they were either derived from a clade B-based isolate (gag H-b; nef-b) or a clade C-based isolate (gag H-c, nef-c). Data in Table 15 shows that T cells from these patients presumably infected with clade B HIV-1 could recognize clade C gag and nef antigens in ELISPOT assay. Correlation analysis further demonstrated that these T cell responses against clade C gag peptide pool were about 60% of the clade B counterpart (FIG. 24), while the T cell responses against clade C nef were about 85% of the clade B counterpart (FIG. 25). These results suggest that cellular immune responses generated in patients infected with clade B HIV-1 can recognize gag and nef antigens derived from clade C HIV-1. These data show that a HIV vaccine, such as a DNA or MRKAd5-based adenoviral vaccine expressing a clade B gag and/or nef antigen will potentially have the ability to provide a prophylactic and/or therapetic advantage on a global scale.

TABLE 15
Responses Shown as the Number of gIFN-Secreting
T Cells per Million PBMCs

| | | gag | | | | | |
|---|---|---|---|---|---|---|---|
| sub- | bleed date | epitope # | mock | | | | |
| ject | (from mapping) | | | gag H-b | gagH-c | nef-b | nef-c |
| #100 | 19-Jul-99 | 12 | 10 | 3950 | 1385 | 1295 | 1300 |
| #101 | 25-Jul-99 | 3 | 15 | 3885 | 1280 | na | 1020 |
| #102 | 25-Jul-99 | 4 | 15 | 1740 | 850 | 1255 | 1785 |
| #104 | 7-Jun-99 | 2 | 5 | 1355 | 1185 | na | 1060 |
| #107 | 11-Oct-99 | 2 | 25 | 3305 | 2795 | 670 | 870 |
| #405 | 11-Jul-99 | 2 | 15 | 4575 | 3180 | 1700 | 1500 |
| #501 | 19-Jul-99 | 2 | 15 | 1100 | 570 | 3365 | 3460 |
| #505 | 18-Jul-99 | 5 | 10 | 2145 | 1725 | 1235 | na |
| #506 | 28-Feb-99 | 2 | 25 | 150 | 45 | 400 | 610 |
| #701 | 28-Mar-99 | 5 | 30 | 7620 | 4775 | 3320 | 2780 |
| #709 | 17-May-99 | 3 | 15 | 2785 | 1945 | 1090 | 1630 |
| #710 | 24-May-99 | 4 | 5 | 1055 | 1080 | 2210 | 2140 |

### EXAMPLE 26

### Characterization and Production of MRKAd5pol and MRKAd5nef Vectors in Roller Bottles

Expansion of nef and pol Adenovectors—Nef and pol CsCl purified MRKAd5 seeds were used to infect roller bottles to produce P4 virus to be used as a seed for further experiments. P4 MRKAd5 pol and nef vectors were used to infect roller bottles at an MOI 280vp/cell, except for hCMV-tpa-nef [E3+] which was infected at an MOI of 125 due to low titers of seed obtained at P4.

TABLE 16
Viral particle concentrations for P5 nef and pol adenovectors

| | AEX Titer | | |
|---|---|---|---|
| | (10¹⁰ vp/ml | AEX Titer | Amplification |
| Adenovector | culture) | (10⁴ vp/cell) | Ratio |
| hCMV-FL-nef [E3+] | 1.1 | 0.9 | 30 |
| mCMV-FL-nef [E3+] | 2.2 | 2.1 | 75 |
| hCMV-tpa-nef [E3+] | 0.07 | 0.1 | 5 |
| mCMV-tpa-nef [E3+] | 1.3 | 0.9 | 35 |
| hCMV-FL-pol [E3+] | 2.7 | 2.1 | 75 |
| hCMV-FL-pol [E3−] | 1.9 | 1.3 | 45 |

Roller Bottle Passaging—Passaging of the pol and nef constructs continued through passage seven. Cell-associated (freeze/thaw lysis) and whole broth (triton-lysis) titers obtained in all passages were very consistent. In general, MRKAd5pol is ca. 70% as productive as MRKAd5gag while MRKAd5nef is ca. 25% as productive as MRKAd5gag. Samples of P7 virus for both constructs were analyzed by V&CB by restriction digest analysis and did not show any rearrangements.

TABLE 17
Passage Six Viral Productivity for MRKAd5pol and MRKAd5nef

| | | Xviable (10⁶ cells/ml), | | | AEX Titer | | | |
|---|---|---|---|---|---|---|---|---|
| | | Viability (%) | | Cell Passage | (Cell Associated) | Titer | Amplification | Triton Lysis Titer |
| | | Infection | Harvest | Number | 10¹⁰ vp/ml culture | 10⁴ vp/cell | Ratio | 10¹⁰ vp/ml culture |
| hCMV-FL-nef [E3+] | pool | 1.22, 85% | | 62 | 0.8 | 0.7 | 25 | 1.6 |
| | 1 | | 0.99, 62% | | | | | |
| | 2 | | 1.10, 72% | | | | | |
| hCMV-FL-pol [E3+] | pool | 1.42, 89% | | 62 | 4.5 | 3.2 | 115 | 7.0 |
| | 1 | | 1.22, 70% | | | | | |
| | 2 | | 1.42, 74% | | | | | |

TABLE 18
Passage Seven Viral Productivity for MRKAd5pol and MRKAd5nef

| | | Xviable (10⁶ cells/ml), | | | AEX Titer | | | |
|---|---|---|---|---|---|---|---|---|
| | | Viability (%) | | Cell Passage | (Cell Associated) | Titer | Amplification | Triton Lysis Titer |
| | | Infection | Harvest | Number | 10¹⁰ vp/ml culture | 10⁴ vp/cell | Ratio | 10¹⁰ vp/ml culture |
| hCMV-FL-nef [E3+] | Pool | 1.33, 90% | | 66 | 1.0 | 0.8 | 29 | 2.1 |
| | 1 | | 0.96, 70% | | | | | |
| | 2 | | 1.18, 73% | | | | | |
| hCMV-FL-pol [E3+] | Pool | 0.90*, | | 56 | 4.2 | 4.7 | 168 | 6.5 |
| | 1 | 90% | 1.18, 88% | | | | | |
| | 2 | | 1.04, 80% | | | | | |

MRK4d5nef and MRKAd5pol Viral Production Kinetics—A timecourse experiment was carried out in roller bottles to determine if the viral production kinetics of the MRKAd5pol and MRKAd5nef vectors were similar to those of MRKAd5gag. PER.C6® cells in roller bottle cultures were infected at an MOI of 280vp/cells with P5 MRKAd5pol, P5 MRKAd5nef and P7 MRKAd5gag; for each adenovector, two infected bottles were sampled at 24, 36, 48, and 60 hours post infection. In addition, two bottles were left unsampled until 48 hpi when they were harvested under the Phase I process conditions. The anion-exchange HPLC viral particle concentrations of the freeze-thaw recovered cell associated virus at the 24, 36, 48, and 60 hpi timepoints are shown in FIG. 29A-B. The QPA titers show a similar trend (data not shown).

Comparison of hCMV- and mCMV-FL-nef—As the titers obtained with the MRKAd5nef construct (hCMV-FL-nef) were lower than those obtained with MRKAd5gag or MRKAd5pol, a viral productivity comparison experiment was performed with mCMV-FL-nef. For each of the two adenovectors (hCMV- and mCMV-FL-nef), two roller bottles were infected at an MOI of 280vp/cell with passage five clarified lysate. The macroscopic and microscopic observations of the four roller bottles were identical at the time of harvest. Analysis of the clarified lysate produced indicated a higher viral particle concentration in the bottles infected with mCMV-FL-nef, as shown in Table 19. It is stipulated that the higher productivity with mCMV promoter driven nef vector is due to lower nef expression levels in PER.C6® cells—experiments are underway at V&CB to measure nef expression levels.

TABLE 19
Passage Six Viral Productivity Comparison of hCMV- and mCMV-FL-nef

| | | Xv (10⁶ | | | | | | |
|---|---|---|---|---|---|---|---|---|
| | | cells/ml), Viability (%) | | Cell Passage | AEX Titer | Titer | Amplification | Triton Lysis Titer |
| | | Infection | Harvest | Number | 10¹⁰ vp/ml culture | 10⁴ vp/cell | Ratio | 10¹⁰ vp/ml culture |
| hCMV-FL-nef | Pool | 1.11, 91% | | 60 | 1.5 | 1.4 | 50 | 2.8 |
| (MRKAd5nef) | 1 | | 1.23, 75% | | | | | |
| | 2 | | 1.34, 74% | | | | | |
| mCMV-FL-nef | Pool | 1.11, 91% | | 60 | 2.3 | 2.1 | 75 | 4.6 |
| | 1 | | 1.49, 84% | | | | | |
| | 2 | | 1.18, 77% | | | | | |

### EXAMPLE 27

### Characterization and Large Scale Production of MRKAd5nef Virus in Bioreactors

Materials and Methods—The experiment of the present example was run twice under the following conditions: 36.5° C., DO 30%, pH 7.30, 150 rpm agitation rate, no sparging, Life Technologies (Gibco, Invitrogen) 293 SFM II (with 6 mM L-glutamine), 0.5M NaOH as base for pH control. During the first run (B20010115), two 10 L stirred vessel bioreactors were inoculated with PER.C6® cells at a concentration of 0.2×10⁶ cells/ml. Cells were grown until they reached a cell concentration of approximately 1×10⁶ cells/ml. The cells were infected with uncloned MRKAd5nef (G2A,LLAA) at a MOI of 280 virus particles (vp)/cell. For the second batch (B20010202), the same procedure as the first run was used, except the cells were infected with cloned MRAd5nef. During both runs, the bioreactors were harvested 48 hours post-infection. Samples were taken and virus concentrations were determined from whole broth (with triton lysis), supernatant, and cell pellets (3×freeze/thaw) with the AEX and QPA assays. Metabolites were measured with BioProfile 250 throughout the process.

TABLE 20
Experimental Conditions

| | |
|---|---|
| Temperature | 36.5° C. |
| DO | 30% |
| PH | 7.30 |
| Agitation | 150 rpm |
| Sparging | None |

TABLE 21
Virus source used for experiments.

| | | Cloned/Uncloned | MOI |
|---|---|---|---|
| Run | Batch ID | MRKAd5nef | (vp/cells) |
| #1 | B20010115-1 | Uncloned | 280 |
| | B20010115-2 | Uncloned | 280 |
| #2 | B20010202-1 | Cloned | 280 |
| | B20010202-2 | Cloned | 280 |

Results—Table 22 and 23 show an the ability to scale up production of MRKAd5nef by growth in a bioreactor.

TABLE 22
Virus Concentration as measured by the AEX assay

| | | Cloned/Uncloned | Virus Concentration @ 48 hpi (1 × 10¹³ vp/L) | | | |
|---|---|---|---|---|---|---|
| Run | Batch ID | MRKAd5nef | Supernatant | Clarified Lysate | Total | Triton Lysate |
| #1 | B20010115-1 | Uncloned | 0.72 | 3.26 | 3.98 | 5.76 |
| | B20010115-2 | Uncloned | 0.38 | 1.67 | 2.05 | 2.46 |
| #2 | B20010202-1 | Cloned | 0.80 | 6.00 | 6.80 | 8.88 |
| | B20010202-2 | Cloned | 0.50 | 6.00 | 6.50 | 8.47 |

TABLE 23
Virus Titers as measured by the QPA assay

| | | | Virus Concentration @ 48 hpi (1 × 10¹¹ IU/L) | | | | |
|---|---|---|---|---|---|---|---|
| | | Cloned/Uncloned | Whole | | Clarified | | Triton |
| Run | Batch ID | MRKAd5nef | Broth | Supernatant | Lysate | Total | Lysate |
| #1 | B20010115-1 | Uncloned | 0.13 | 1.12 | 1.76 | 2.88 | 11.28 |
| | B20010115-2 | Uncloned | 0.14 | 0.73 | 1.54 | 2.27 | 5.86 |
| #2 | B20010202-1 | Cloned | 0.14 | 0.97 | 1.62 | 2.69 | 11.89 |
| | B20010202-2 | Cloned | 0.14 | 1.17 | 1.70 | 2.97 | 12.47 |

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

### EXAMPLE 28

### MRKAd5HV-1gag Boosting of DNA-Primed Animals

Groups of 3-5 rhesus macaques were immunized with (a) 5 mgs of VI Jns-Flgag (pVIJnsCMV(no intron)-FL-gag-bGHpA), (b) 5 mgs of V1Jns-Flgag formulated with 45 mgs of a non-ionic block copolymer CRL1005, or (c) 5 mgs of V1Jns-Flgag formulated with 7.5 mgs of CRL1005 and 0.6 mM benzalkonium chloride at weeks 0, 4, and 8. All animals received a single dose of 10e7 viral particles (vp) of the MRKAd5HIV-1 gag at week 26. Note: 10e7 is too low to prime or boost effectively when used as a single modality (dose is selected to mimic preexposure to adenovirus); see FIG. 32.

Blood samples were collected from all animals at several time points and peripheral blood mononuclear cells (PBMCs) were prepared using standard Ficoll method. The PBMCs were counted and analyzed for gamma-interferon secretion using the ELISpot assay (Table 24). For each monkey, the PBMCs were incubated overnight either in the absence (medium) or presence of a pool (called “gag H”) of 50 20-aa long peptides that encompass the entire HIV-1 gag sequence.

The results indicate that MRKAd5HIV-1gag was very effective in boosting the T cell immune responses in these monkeys. At week 28 or 2 weeks after the viral boost, the number of gag-specific T cells per million PBMCs increased 2-48 fold compared to the levels observed at week 24 or 2 weeks prior to the boost.

The PBMCs were also analyzed by intracellular gamma-interferon staining prior to (at week 10) and after the MRKAd5gag boost (at week 30). The results for select animals are shown on FIG. 31. The results indicate that (a) immunization with DNA/adjuvant formulation elicited T cell responses which can either be balanced, CD4⁺-biased or CD8⁺-biased, and (b) boosting with the MRKAd5gag construct produced in all cases a strongly CD8⁺-biased response. These results suggest that boosting with MRKAd5HIV-1gag construct is able to improve the levels of antigen-specific CD8⁺ T cells.

### EXAMPLE 29

### Construction of Gagpol Fusion for MRKAd5Gagpol Fusion Constructs

The open reading frames for the codon-optimized HIV-1 gag gene was fused directly to the open reading frame of the IA pol gene (consisting of RT, RNAseH and integrase domains) by stepwise PCR. Because the gene (SEQ ID NO: 34) does not include the protease gene and the frameshift sequence, it encodes a single polypeptide of the combined size of p55, RT, RNAse H and integrase (1350 amino acids; SEQ ID NO: 35).

The fragment that extends from the BstEII site within the gag gene to the last non-stop codon was ligated via PCR to a fragment that extends from the start codon of the IApol to a unique BamHI site. This fragment was digested with BstEII and BamHI. Construction of gag-IApol fusion was achieved via three-fragment ligation involving the PstI-BstEII gag digestion fragment, the BstEII/BamHI digested PCR product and long PstI/BamHI V1R-FLpol backbone fragment.

The MRKAd5-gagpol adenovirus vector was constructed using the Bg1II fragment of the V1R-gagpol containing the entire ORF of gag-IApol fusion gene.

### EXAMPLE 30

### Immunogenicity Studies in Non-Human Primates

Cohorts of three (3) macaques were immunized with 10e8 or 10e10 viral particles (vp) of one of the following MRKAd5 HIV-1 vaccines: (1) MRKAd5gag; (2) MRKAd5pol; (3) MRKAd5nef; (4) a mixture containing equal amounts of MRKAd5gag, MRKAd5pol, and MRKAd5nef, or (5) a mixture of equal amounts of MRKAd5gagpol and MRKAd5nef. The vaccines were administered at weeks 0 and 4.

The T cell responses against each of the HIV-1 antigens were assayed by IFN-gamma ELISpot assay using pools of 20-aa peptides that encompass the entire protein sequence of each antigen. The results (Table 25) are expressed as the number of spot-forming cells (sfc) per million peripheral blood mononuclear cells (PBMC) that respond to each of the peptide pools.

Results indicate the following observations: (1) each of the single gene constructs (MRKAd5gag, MRKAd5pol, or MRKAd5nef) is able to elicit high levels of antigen-specific T cells in monkeys; (2) the single-gene MRKAd5 constructs can be mixed as a multi-cocktail formulation capable of eliciting very broad T cell responses against gag, pol, and nef; (3) the MRKAd5 vector expressing the fusion protein of gag plus IA pol is capable of inducing strong T cell responses to both gag and pol.

TABLE 25
Evaluation of Mixtures of MRKAd5 vectors expressing humanized HIV-1 gag, pol,
gagpol, nef in rhesus macaques

| | Vaccine | | T = 6 wks | | | | |
|---|---|---|---|---|---|---|---|
| Grp # | T = 0, 4 wks | Monk # | Mock | Gag H | Pol-1 | Pol-2 | Nef |
| 1 | MRKAd5 gag | CB9V | 0 | 15 | — | — | — |
| | 10\hat{ }10 vp | CD19 | 0 | 374 | — | — | — |
| | | 109H | 1 | 843 | — | — | — |
| 2 | MRKAd5 gag | 99D130 | 1 | 948 | — | — | — |
| | 10\hat{ }8 vp | W277 | 16 | 324 | — | — | — |
| | | 143H | 4 | 595 | — | — | — |
| 3 | MRKAd5 pol | CC1X | 4 | — | 46 | 256 | — |
| | 10\hat{ }10 vp | AW3W | 3 | — | 463 | 550 | — |
| | | AV43 | 6 | — | 95 | 1333 | — |
| 4 | MRKAd5 pol | AW38 | 1 | — | 19 | 30 | — |
| | 10\hat{ }8 vp | CC8K | 0 | — | 50 | 995 | — |
| | | CC21 | 1 | — | 33 | 436 | — |
| 5 | MRKAd5 nef | 076Q | 9 | — | — | — | 1204 |
| | 10\hat{ }10 vp | 091Q | 4 | — | — | — | 85 |
| | | 083Q | 0 | — | — | — | 176 |
| 6 | MRKAd5 nef | 00C029 | 1 | — | — | — | 114 |
| | 10\hat{ }8 vp | 98D022 | 6 | — | — | — | 170 |
| | | 98D160 | 3 | — | — | — | 198 |
| 7 | MRKAd5gag + MRKAd5pol + MRKAd5nef | 99D251 | 3 | 206 | 15 | 193 | 120 |
| | 10\hat{ }10 vp each | 05H | 3 | 135 | 21 | 9 | 638 |
| | | 00C016 | 3 | 26 | 4 | 51 | 23 |
| 8 | MRKAd5gag + MRKAd5pol + MRKAd5nef | 99D215 | 1 | 171 | 18 | 193 | 240 |
| | 10\hat{ }8 vp each | 81H | 5 | 73 | 6 | 14 | 243 |
| | | 12H | 8 | 1140 | 115 | 811 | 719 |
| 9 | MRKAd5gagpol + MRKAd5 nef | 99D211 | 0 | 83 | 56 | 838 | 725 |
| | 10\hat{ }10 vp each | 22H | 4 | 385 | 119 | 1194 | 1915 |
| | | 61H | 4 | 343 | 11 | 765 | 853 |
| 10 | MRKAd5gagpol + MRKAd5 nef | 34H | 3 | 78 | 19 | 5 | 75 |
| | 10\hat{ }8 vp each | 48H | 1 | 65 | 105 | 46 | 43 |
| | | 70H | 5 | 158 | 15 | 220 | 191 |

Indicated are numbers of spot-forming cells per million PBMCS against the peptide pools.
Mock, no peptides;
Gag H, fifty 20-aa peptides encompassing p55 sequence;
Pol-1, 20-aa peptides representing N-terminal half of IA pol;
Pol-2, 20-aa peptides representing the carboxy-terminal half of IA pol;
Nef, 20-aa peptides encompassing the entire wild-type nef sequence.
Responses to the antigens prior to the first immunization did not exceed 40 sfc/10\hat{ }6 PBMC.

What is claimed is:

1. An HIV vaccine composition comprising recombinant, replication-defective adenovirus particles harvested and purified from a cell line transfected with a recombinant adenoviral vector; said cell line which expresses adenovirus E1 protein at complementing levels; and said recombinant adenoviral vector which is at least partially deleted in E1 and devoid of E1 activity, and comprises:
a) an adenovirus cis-acting packaging region corresponding to from about base pair 1 to between from about base pair 400 to about base pair 458 of a wildtype adenovirus genome; and
b) at least one gene encoding an HIV protein selected from the group consisting of HIV gag, nef, pol, and immunologically relevant modifications thereof.

2. An HIV vaccine composition of claim 1 which comprises a physiologically acceptable carrier.

3. A method of generating a cellular-mediated immune response against HIV in an individual comprising administering to the individual a vaccine composition of claim 1.

4. A method according to claim 3 which further comprises administration to the individual a DNA plasmid vaccine, optionally administered with a biologically effective adjuvant, protein or other agent capable of increasing the immune response.

5. A method according to claim 4 wherein the DNA plasmid vaccine is administered to the individual prior to administration of the vaccine composition.

6. A method according to claim 3 wherein the vaccine composition is preceded by a vaccine composition comprising purified recombinant, replication-defective adenovirus particles of a different serotype.

7. A method according to claim 3 which comprises administering and readministering the vaccine composition to the individual.

8. An HIV vaccine composition comprising recombinant, replication-defective adenovirus particles harvested and purified from a cell line transfected with a recombinant adenoviral vector; said cell line which expresses adenovirus E1 protein at complementing levels; and said recombinant adenoviral vector which is at least partially deleted in E1 and devoid of E1 activity, and comprises:
a) an adenovirus cis-acting packaging region corresponding to from about base pair 1 to about base pair 450 of a wildtype adenovirus genome;
b) a region corresponding to from about base pair 3511 to about base pair 5798 of a wildtype adenovirus genome; and
c) a gene expression cassette comprising
i) SEQ ID NO: 27;
ii) a heterologous promoter operatively linked to i); and
iii) a transcription termination sequence;
wherein the vector has a deletion corresponding to from about base pair 451 to about base pair 3510 of a wildtype adenovirus genome.

9. An HIV vaccine composition of claim 8 which comprises a physiologically acceptable carrier.

10. A method of generating a cellular-mediated immune response against HIV in an individual comprising administering to the individual a vaccine composition of claim 8.

11. A method according to claim 10 which further comprises administration to the individual a DNA plasmid vaccine, optionally administered with a biologically effective adjuvant, protein or other agent capable of increasing the immune response.

12. A method according to claim 11 wherein the DNA plasmid vaccine is administered to the individual prior to administration of the vaccine composition.

13. A method according to claim 10 wherein the vaccine composition is preceded by a vaccine composition comprising purified recombinant, replication-defective adenovirus particles of a different serotype.

14. A method according to claim 10 which comprises administering and readministering the vaccine composition to the individual.

15. An HIV vaccine composition comprising recombinant, replication-defective adenovirus particles harvested and purified from a cell line transfected with a recombinant adenoviral vector; said cell line which expresses adenovirus E1 protein at complementing levels; and said recombinant adenoviral vector which is at least partially deleted in E1 and devoid of E1 activity, and comprises:
a) an adenovirus cis-acting packaging region corresponding to from about base pair 1 to about base pair 450 of a wildtype adenovirus genome;
b) a region corresponding to from about base pair 3511 to about base pair 5798 of a wildtype adenovirus genome; and
c) a gene expression cassette comprising
i) a nucleotide sequence selected from the group consisting of SEQ ID NO: 1, SEQ ID NO: 3, SEQ ID NO: 5 and SEQ ID NO: 7;
ii) a heterologous promoter operatively linked to i); and
iii) a transcription termination sequence;
wherein the vector has a deletion corresponding to from about base pair 451 to about base pair 3510 of a wildtype adenovirus genome;
and, further, wherein the HIV vaccine composition either comprises, or is administered simultaneously with a vaccine composition which comprises, nucleic acid encoding an HIV-1 Gag antigen.

16. An HIV vaccine composition of claim 15 which comprises a physiologically acceptable carrier.

17. A method of generating a cellular-mediated immune response against HIV in an individual comprising administering to the individual a vaccine composition of claim 15.

18. A method according to claim 17 which further comprises administration to the individual a DNA plasmid vaccine, optionally administered with a biologically effective adjuvant, protein or other agent capable of increasing the immune response.

19. A method according to claim 18 wherein the DNA plasmid vaccine is administered to the individual prior to administration of the vaccine composition.

20. A method according to claim 17 wherein the vaccine composition is preceded by a vaccine composition comprising purified recombinant, replication-defective adenovirus particles of a different serotype.

21. A method according to claim 17 which comprises administering and readministering the vaccine composition to the individual.

22. An HIV vaccine composition comprising recombinant, replication-defective adenovirus particles harvested and purified from a cell line transfected with a recombinant adenoviral vector; said cell line which expresses adenovirus E1 protein at complementing levels; and said recombinant adenoviral vector which is at least partially deleted in E1 and devoid of E1 activity, and comprises:
a) an adenovirus cis-acting packaging region corresponding to from about base pair 1 to about base pair 450 of a wildtype adenovirus genome;
b) a region corresponding to from about base pair 3511 to about base pair 5798 of a wildtype adenovirus genome; and
c) a gene expression cassette comprising
i) a nucleotide sequence selected from the group consisting of SEQ ID NO: 9, SEQ ID NO: 11, SEQ ID NO: 13 and SEQ ID NO: 15;
ii) a heterologous promoter operatively linked to i); and
iii) a transcription termination sequence;
wherein the vector has a deletion corresponding to from about base pair 451 to about base pair 3510 of a wildtype adenovirus genome.

23. An HIV vaccine composition of claim 22 which comprises a physiologically acceptable carrier.

24. A method of generating a cellular-mediated immune response against HIV in an individual comprising administering to the individual a vaccine composition of claim 22.

25. A method according to claim 24 which further comprises administration to the individual a DNA plasmid vaccine, optionally administered with a biologically effective adjuvant, protein or other agent capable of increasing the immune response.

26. A method according to claim 25 wherein the DNA plasmid vaccine is administered to the individual prior to administration of the vaccine composition.

27. A method according to claim 24 wherein the vaccine composition is preceded by a vaccine composition comprising purified recombinant, replication-defective adenovirus particles of a different serotype.

28. A method according to claim 24 which comprises administering and readministering the vaccine composition to the individual.

29. A multivalent adenovirus vaccine composition which comprises recombinant, replication-defective adenovirus particles harvested and purified from a cell line transfected with a recombinant adenoviral vector; said cell line which expresses adenovirus E1 protein at complementing levels; said recombinant adenoviral vector which is at least partially deleted in E1 and devoid of E1 activity, and comprises:
a) an adenovirus cis-acting packaging region corresponding to from about base pair 1 to between from about base pair 400 to about base pair 458 of a wildtype adenovirus genome; and
b) gene expression cassette or cassettes comprising nucleotide sequences encoding HIV proteins selected from the group consisting of:
i) gag, pol, and nef, expressed independently from three individual vectors;
ii) gag, pol, and nef, expressed independently from one vector with the encoding nucleic acid sequences operatively linked to distinct promoters and transcription termination sequences;
iii) gag, pol, and nef, expressed via two vectors, one expressing a pol-nef fusion, and another expressing gag;
iv) gag, pol, and nef, expressed via two vectors, one expressing a gag-pol fusion and another expressing nef;
v) gag, pol and nef, expressed via two vectors, one expressing a nef-gag fusion and another expressing pol;
vi) gag, pol, and nef, expressed via one vector expressing a gag-pol-nef fusion;
vii) gag and pol, expressed independently from two individual vectors;
viii) gag and pol, expressed independently from one vector with the encoding nucleic acid sequences operatively linked to distinct promoters and transcription termination sequences;
ix) pol and nef, expressed independently from two individual vectors;
x) pol and nef, expressed independently from one vector with the encoding nucleic acid sequences operatively linked to distinct promoters and transcription termination sequences;
xi) nef and gag, expressed independently from two individual vectors;
xii) nef and gag, expressed independently from one vector with the encoding nucleic acid sequences operatively linked to distinct promoters and transcription termination sequences;
xiii) gag and pol, expressed via one vector expressing a gag-pol fusion;
xiv) pol and nef, expressed via one vector expressing a pol-nef fusion; and
xv) nef and gag, expressed via one vector expressing a nef-gag fusion.

30. A multivalent adenovirus vaccine composition in accordance with claim 29 wherein the gag-pol fusion comprises SEQ ID NO: 35.

31. A multivalent adenovirus vaccine composition in accordance with claim 29 wherein the fused sequences have the encoding nucleic acid sequences operatively linked to distinct promoters and transcription termination sequences.

32. A multivalent adenovirus vaccine composition in accordance with claim 29 wherein the fused sequences have the encoding nucleic acid sequences operatively linked to a single promoter; and the encoding nucleic acid sequences operatively linked by an internal ribosome entry sequence (“IRES”).
